(19) United States
(12) Patent Application Publication
(10) Pub. No.: US 2023/0014907 A1
(43) Pub. Date: Jan. 19, 2023

(54) INHIBITORS OF APOL1 AND METHODS OF USING SAME

(71) Applicant: Vertex Pharmaceuticals Incorporated, Boston, MA (US)

(72) Inventors: Leslie A. Dakin, Framingham, MA (US); Timothy J. Senter, Arlington, MA (US); Jingrong Cao, Newton, MA (US); Jon H. Come, Cambridge, MA (US); Francois Denis, St-Lazare (CA); Warren A. Dorsch, Waltham, MA (US); Anne Fortier, Jamaica Plain, MA (US); Martine Hamel, Laval (CA); Elaine B. Krueger, Milton, MA (US); Brian Ledford, Norton, MA (US); Francois Maltais, Stoneham, MA (US); Suganthini S. Nanthakumar, Newton, MA (US); Olivier Nicolas, Montreal (CA); Camil E. Sayegh, Belmont, MA (US); Tiansheng Wang, Concord, MA (US)

(73) Assignee: Vertex Pharmaceuticals Incorporated

(21) Appl. No.: 17/345,256

(22) Filed: Jun. 11, 2021

Related U.S. Application Data
(60) Provisional application No. 63/038,276, filed on Jun. 12, 2020.

(51) Int. Cl. C07D 209/18; C07D 403/12; C07D 405/12; C07D 401/12; C07D 403/06; C07D 413/12; C07D 401/06; C07D 417/12; C07D 409/12; C07D 209/40; C07D 209/16; C07D 209/12

(52) U.S. Cl. CPC C07D 209/18; C07D 403/12; C07D 405/12; C07D 401/12; C07D 403/06; C07D 209/12; C07D 401/06; C07D 417/12; C07D 409/12; C07D 209/40; C07D 209/16; C07D 413/12

(57) ABSTRACT

The disclosure provides compounds of Formula I,

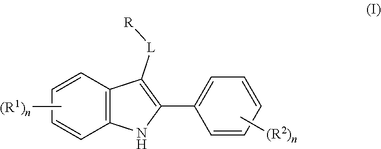

deuterated derivatives of those compounds, and pharmaceutically acceptable salts of those compounds and derivatives, compositions comprising the same, and methods of using the same, including use in treating APOL1 mediated kidney disease.

This application claims the benefit of priority of U.S. Provisional Application No. 63/038,276, filed Jun. 12, 2020, which is incorporated by reference herein in its entirety.

This disclosure provides compounds that inhibit apolipoprotein L1 (APOL1) and methods of using those compounds to treat APOL1 mediated kidney disease, including focal segmental glomerulosclerosis (FSGS) and/or non-diabetic kidney disease (NDKD). In some embodiments, the FSGS and/or NDKD is associated with common APOL1 genetic variants (G1: S342G:I384M and G2: N388del:Y389del).

FSGS is a disease of the podocyte (glomerular visceral epithelial cells) responsible for proteinuria and progressive decline in kidney function. NDKD is a disease characterized by hypertension and progressive decline in kidney function. Human genetics support a causal role for the G1 and G2 APOL1 variants in inducing kidney disease. Individuals with two APOL1 risk alleles are at increased risk of developing end-stage kidney disease (ESKD), including FSGS, human immunodeficiency virus (HIV)-associated nephropathy, NDKD, arterionephrosclerosis, lupus nephritis, microalbuminuria, and chronic kidney disease. See, P. Dummer et al., Semin Nephrol. 35(3): 222-236 (2015).

APOL1 is a 44 kDa protein that is only expressed in humans, gorillas, and baboons. APOL1 is produced mainly by the liver and contains a signal peptide that allows for secretion into the bloodstream, where it circulates bound to a subset of high-density lipoproteins. APOL1 is responsible for protection against the invasive parasite, Trypanosoma brucei brucei (T. b. brucei). APOL1 G1 and G2 variants confer additional protection against trypanosoma species that cause sleeping sickness. Although normal plasma concentrations of APOL1 are relatively high and can vary at least 20-fold in humans, circulating APOL1 is not causally associated with kidney disease.

However, APOL1 in the kidney is thought to be responsible for the development of kidney diseases, including FSGS and NDKD. Under certain circumstances, APOL1 protein synthesis can be increased by approximately 200-fold by pro-inflammatory cytokines such as interferons or tumor necrosis factor-α. In addition, several studies have shown that APOL1 protein can form pH-gated Na⁺/K⁺ pores in the cell membrane, resulting in a net efflux of intracellular K⁺, ultimately resulting in activation of local and systemic inflammatory responses, cell swelling, and death.

The risk of ESKD is substantially higher in people of recent sub-Saharan African ancestry as compared to those of European ancestry. In the United States, ESKD is responsible for nearly as many lost years of life in women as from breast cancer and more lost years of life in men than from colorectal cancer. Currently, FSGS and NDKD are managed with symptomatic treatment (including blood pressure control using blockers of the renin angiotensin system), and patients with FSGS and heavy proteinuria may be offered high dose steroids. Corticosteroids induce remission in a minority of patients and are associated with numerous and, at times, severe side effects, and are often poorly tolerated. These patients, and particularly individuals of recent sub-Saharan African ancestry with two APOL1 risk alleles, experience faster disease progression leading to ESKD.

Thus, there is an unmet medical need for treatment for APOL1 mediated kidney diseases, including FSGS, NDKD, and ESKD. In view of evidence that APOL1 plays a causative role in inducing and accelerating the progression of kidney disease, inhibition of APOL1 should have a positive impact on patients with APOL1 mediated kidney disease, particularly those who carry two APOL1 risk alleles (i.e., are homozygous or compound heterozygous for the G1 or G2 alleles).

One aspect of the disclosure provides at least one entity (e.g., at least one compound, deuterated derivative, or pharmaceutically acceptable salt) chosen from compounds of Formula I:

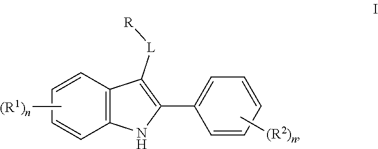

deuterated derivatives thereof, and pharmaceutically acceptable salts of any of the foregoing, which can be employed in the treatment of diseases mediated by APOL1, such as, e.g., FSGS and NDKD, wherein:
(i) R is selected from —C(O)NR³R⁴, —NR⁵C(O)R³, —NR⁵C(O)NR³R⁴, —NR³R⁴, —OR³, —NR⁵—SO₂R³, —OC(O)NR³R⁴, —C(O)OR³,

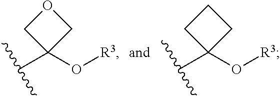

(ii) L is selected from divalent C₁-C₆ linear and branched alkyl (e.g., divalent C₁-C₆ linear and C₃-C₆ branched alkyl), divalent C₂-C₆ linear and branched alkenyl (e.g., divalent C₂-C₆ linear and C₃-C₆ branched alkenyl), divalent C₂-C₆ linear and branched alkynyl (e.g., divalent C₂-C₆ linear and C₃-C₆ branched alkynyl), and divalent 1- to 7-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;
   (iii) each R¹ is independently selected from:
halogen,
hydroxy,
thiol,
amino,
cyano,
C₁-C₆ linear, branched, and cyclic alkyl (e.g., C₁-C₆ linear, C₃-C₆ branched, and C₃-C₆ cyclic alkyl) optionally substituted with 1-3 groups independently selected from hydroxy and halogen,
C₂-C₆ linear, branched, and cyclic alkenyl (e.g., C₂-C₆ linear, C₃-C₆ branched, and C₃-C₆ cyclic alkenyl),
C₁-C₆ linear, branched, and cyclic alkoxy (e.g., C₁-C₆ linear, C₂-C₆ branched, and C₂-C₆ cyclic alkoxy) optionally substituted with 1-3 groups independently selected from halogen,
C₁-C₆ linear, branched, and cyclic thioalkyl (e.g., C₁-C₆ linear, C₂-C₆ branched, and C₂-C₆ cyclic thioalkyl) optionally substituted with 1-3 groups independently selected from halogen, and
C₁-C₆ linear, branched, and cyclic aminoalkyl optionally substituted with 1-3 groups independently selected from halogen,
or two R¹ groups, together with the carbon atoms to which they are attached, may form a C₄-C₈ cycloalkyl, aryl, or heteroaryl;
   (iv) each R² is independently selected from:
halogen,
hydroxy,
thiol,
amino,
cyano,
C₁-C₆ linear, branched, and cyclic alkyl (e.g., C₁-C₆ linear, C₃-C₆ branched, and C₃-C₆ cyclic alkyl) optionally substituted with 1-3 groups independently selected from hydroxy and halogen,
C₂-C₄ linear, branched, and cyclic alkenyl (e.g., C₂-C₄ linear, C₃-C₄ branched, and C₃-C₄ cyclic alkenyl),
C₁-C₆ linear, branched, and cyclic alkoxy (e.g., C₁-C₆ linear, C₂-C₆ branched, and C₂-C₆ cyclic alkoxy) optionally substituted with 1-3 groups independently selected from halogen,
C₁-C₄ linear, branched, and cyclic thioalkyl (e.g., C₁-C₄ linear, C₂-C₄ branched, and C₂-C₄ cyclic thioalkyl) optionally substituted with 1-3 groups independently selected from halogen, and
C₁-C₄ linear, branched, and cyclic aminoalkyl optionally substituted with 1-3 groups independently selected from halogen,
   (v) each n is independently selected from 0, 1, 2, 3, and 4;
   (vi) R³ and R⁴ are independently selected from:
hydrogen,
C₁-C₆ linear and branched alkylsulfonyl (e.g., C₁-C₆ linear and C₂-C₆ branched alkyl sulfonyl),
C₂-C₆ linear and branched alkenyl (e.g., C₂-C₆ linear and C₃-C₆ branched alkenyl),
amino optionally substituted with 1-2 groups independently selected from C₁-C₆ linear and branched alkyl (e.g., C₁-C₆ linear and C₃-C₆ branched alkyl) and C₃-C₆ cycloalkyl,
amide optionally substituted with 1-2 groups independently selected from C₁-C₃ alkyl,
C₁-C₆ linear and branched alkoxy (e.g., C₁-C₆ linear and C₂-C₆ branched alkoxy) optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl (e.g., C₃-C₆ cyclic alkyl) optionally substituted with 1-2 groups independently selected from:
   halogen,
   hydroxy,
   oxo,
   amino optionally substituted with 1-2 groups independently selected from hydrogen and C₁-C₆ linear or branched alkyl (e.g., C₁-C₆ linear or C₃-C₆ branched alkyl),
   aryl optionally substituted with 1-2 groups independently selected from halogen,
   C₁-C₆ linear and branched alkyl groups (e.g., C₁-C₆ linear and C₃-C₆ branched alkyl groups) (which may be further substituted with 1-3 groups independently selected from hydroxy, oxo, halogen, and C₁-C₆ linear and branched alkoxy groups (e.g., C₁-C₆ linear and C₂-C₆ branched alkoxy groups)),
   carbamate optionally substituted with 1-2 groups independently selected from C₁-C₆ linear and branched alkyl (e.g., C₁-C₆ linear and C₃-C₆ branched alkyl),
   C₁-C₆ linear and branched alkoxy (e.g., C₁-C₆ linear and C₂-C₆ branched alkoxy), and
   amide,
4- to 10-membered heterocyclyl optionally substituted with 1-3 groups independently selected from:
   halogen,
   oxo,
   hydroxy,
   amino, and
   C₁-C₆ linear and branched alkyl (e.g., C₁-C₆ linear and C₃-C₆ branched alkyl) (which may be further substituted with 1-2 groups independently selected from hydroxy, oxo, and C₁-C₆ linear and branched alkoxy (e.g., C₁-C₆ linear and C₂-C₆ branched alkoxy)),
aryl optionally substituted with 1-3 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy groups (e.g., C₁-C₆ linear and C₂-C₆ branched alkoxy groups)),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino), and
C₁-C₆ linear and branched alkyl groups (e.g., C₁-C₆ linear and C₃-C₆ branched alkyl groups), wherein the alkyl groups are optionally substituted with 1-4 groups independently selected from:
   amino groups optionally substituted with 1-2 groups independently selected from hydroxy, C₁-C₆ linear, branched, and cyclic alkyl (e.g., C₁-C₆ linear, C₃-C₆ branched, and C₃-C₆ cyclic alkyl) (which may be further substituted with 1-2 oxo), and C₁-C₆ linear and branched alkylsulfonyl (e.g., C₁-C₆ linear and C₂-C₆ branched alkylsulfonyl),
   hydroxy,
   oxo,
   cyano,
   carboxylic acid,
   sulfonic acid,
   —O-heteroaryl,
   carbamate optionally substituted with 1-2 groups independently selected from C₁-C₆ linear and branched alkyl (e.g., C₁-C₆ linear and C₃-C₆ branched alkyl),
   halogen,
   amido optionally substituted with 1-2 groups independently selected from hydroxy, C₁-C₆ linear, branched, and cyclic alkyl groups (e.g., C₁-C₆ linear, C₃-C₆ branched, and C₃-C₆ cyclic alkyl groups), and C₁-C₆ linear, branched, and cyclic hydroxyalkyl (e.g., C₁-C₆ linear, C₃-C₆ branched, and C₃-C₆ cyclic hydroxyalkyl groups),
   C₃-C₆ cyclic alkyl optionally substituted with 1-2 groups independently selected from halogen, hydroxy, C₁-C₆ linear and branched hydroxyalkyl (e.g., C₁-C₆ linear and C₃-C₆ branched hydroxyalkyl), C₁-C₆ linear and branched alkoxy (e.g., C₁-C₆ linear and C₂-C₆ branched alkoxy), and carbamate (which may be further substituted with 1-2 groups independently selected from C₁-C₆ linear and branched alkyl (e.g., C₁-C₆ linear and C₃-C₆ branched alkyl)),
   C₁-C₆ linear and branched alkynyl (e.g., C₂-C₆ linear and branched alkynyl, e.g., C₂-C₆ linear and C₃-C₆ branched alkynyl),
   C₁-C₆ linear and branched alkoxy (e.g., C₁-C₆ linear and C₂-C₆ branched alkoxy) optionally substituted with 1-2 hydroxy,
   C₁-C₆ linear and branched alkylsulfonyl (e.g., C₁-C₆ linear and C₂-C₆ branched alkylsulfonyl),
   aryl optionally substituted with 1-2 groups independently selected from halogen groups, hydroxy, and C₁-C₆ linear and branched alkyl groups (e.g., C₁-C₆ linear and C₃-C₆ branched alkyl groups) (which may be further substituted with 1-2 groups independently selected from hydroxy and C₁-C₆ linear and branched alkoxy groups (e.g., C₁-C₆ linear and C₂-C₆ branched alkoxy groups)),
   carbonyl-(4-methylpiperazin-1-yl),
   carbonyl-(N-morpholino),
   4- to 10-membered heterocyclyl optionally substituted with 1-3 groups independently selected from halogen, oxo, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy (e.g., C₁-C₆ linear and C₂-C₆ branched alkoxy)), and
   4- to 10-membered heteroaryl optionally substituted with 1-3 groups independently selected from halogen, oxo, hydroxy, and C₁-C₆ linear, branched, and cyclic alkyl (e.g., C₁-C₆ linear, C₃-C₆ branched, and C₃-C₆ cyclic alkyl) (which may be further substituted with 1-3 groups independently selected from halogen, hydroxy, and C₁-C₆ linear and branched alkoxy (e.g., C₁-C₆ linear and C₂-C₆ branched alkoxy)),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl (e.g., C₁-C₆ linear, C₃-C₆ branched, and C₃-C₆ cyclic alkyl groups),
halogen,
hydroxy,
oxo,
C₁-C₆ linear, branched, and cyclic alkyl (e.g., C₁-C₆ linear, C₃-C₆ branched, and C₃-C₆ cyclic alkyl groups) optionally substituted with 1-2 groups independently selected from hydroxy, amino, C₁-C₆ linear, branched, and cyclic alkoxy groups (e.g., C₁-C₆ linear, C₂-C₆ branched, and C₂-C₆ cyclic alkoxy groups), and carbamate (which may be further substituted with 1-2 groups independently selected from C₁-C₆ linear and branched alkyl (e.g., C₁-C₆ linear and C₃-C₆ branched alkyl)),
amide optionally substituted with 1-2 groups independently selected from C₁-C₆ linear and branched alkyl (e.g., C₁-C₆ linear and C₃-C₆ branched alkyl),
carbamate optionally substituted with 1-2 groups independently selected from C₁-C₆ linear and branched alkyl (e.g., C₁-C₆ linear and C₃-C₆ branched alkyl),
carboxamide optionally substituted with 1-2 groups independently selected from C₁-C₆ linear and branched alkyl (e.g., C₁-C₆ linear and C₃-C₆ branched alkyl),
C₁-C₆ linear and branched alkoxy groups (e.g., C₁-C₆ linear and C₂-C₆ branched alkoxy groups) optionally substituted with 1-2 groups independently selected from C₁-C₆ linear, branched, and cyclic alkyl (e.g., C₁-C₆ linear, C₃-C₆ branched, and C₃-C₆ cyclic alkyl) and heterocyclyl,
4- to 10-membered heterocyclyl optionally substituted with 1-2 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy (e.g., C₁-C₆ linear and C₂-C₆ branched alkoxy)), and
4- to 10-membered heteroaryl optionally substituted with 1-2 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy groups (e.g., C₁-C₆ linear and C₂-C₆ branched alkoxy groups));

and

(vii) R⁵ is selected from hydrogen and C₁-C₆ linear or branched alkyl (e.g., C₁-C₆ linear or C₃-C₆ branched alkyl). In certain embodiments, the following compounds are excluded from Formula I:

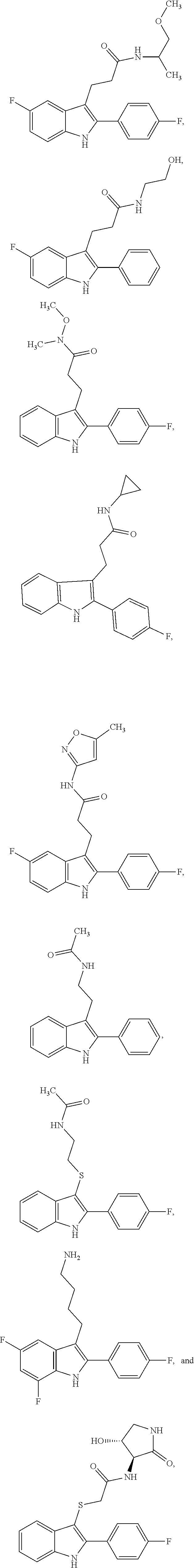

and compounds where -L-R in Formula I is

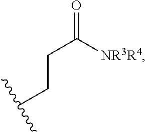

and R³ and R⁴ are

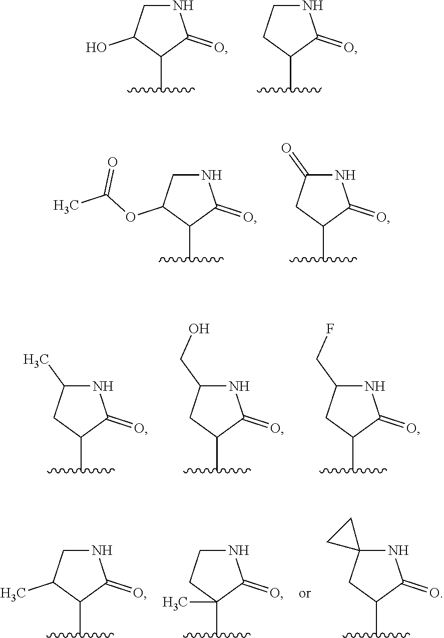

In some embodiments, when L is a divalent C₂ linear alkyl optionally substituted with 1-2 groups independently selected from methyl, halogen, and hydroxy and R is —NR³R⁴, then R³ and R⁴ are not

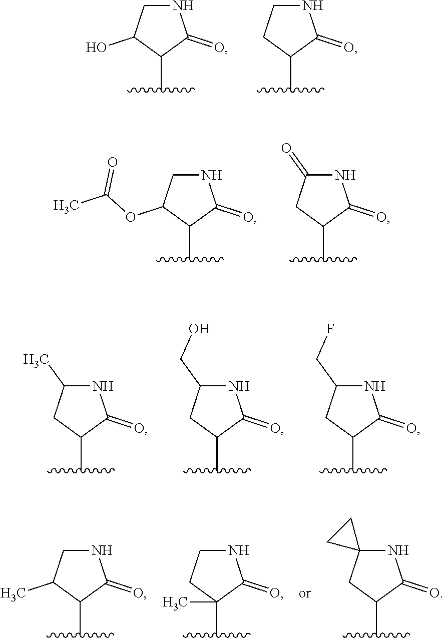

In some embodiments, when L is a divalent C₂ linear alkyl optionally substituted with 1-2 groups independently selected from methyl, halogen, and hydroxy and R is —NR³R⁴, then R³ and R⁴ are not

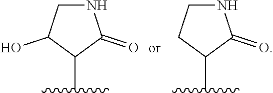

In some embodiments, L is selected from divalent C₁-C₆ linear and branched alkyl, divalent C₂-C₆ linear and branched alkenyl, divalent C₂-C₆ linear and branched alkynyl, and divalent 1- to 6-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;

each R² is independently selected from:
halogen,
hydroxy,
thiol,
amino,
cyano,
C₁-C₄ linear, branched, and cyclic alkyl optionally substituted with 1-3 groups independently selected from hydroxy and halogen,
C₂-C₄ linear, branched, and cyclic alkenyl,
C₁-C₄ linear, branched, and cyclic alkoxy optionally substituted with 1-3 groups independently selected from halogen,
C₁-C₄ linear, branched, and cyclic thioalkyl optionally substituted with 1-3 groups independently selected from halogen, and
C₁-C₄ linear, branched, and cyclic aminoalkyl optionally substituted with 1-3 groups independently selected from halogen; and

R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl and C₃-C₆ cycloalkyl,
amide optionally substituted with 1-2 groups independently selected from C₁-C₃ alkyl, and
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl optionally substituted with 1-2 groups independently selected from:
   halogen,
   hydroxy,
   oxo,
   amino optionally substituted with 1-2 groups independently selected from hydrogen and C₁-C₆ linear or branched alkyl,
   aryl optionally substituted with 1-2 groups independently selected from halogen,
   C₁-C₆ linear and branched alkyl groups (which may be further substituted with 1-3 groups independently selected from hydroxy, oxo, halogen, and C₁-C₆ linear and branched alkoxy groups),
   carbamate optionally substituted with 1-2 groups independently selected from C₁-C₆ linear and branched alkyl,
   C₁-C₆ linear and branched alkoxy, and
   amide,
4- to 10-membered heterocyclyl optionally substituted with 1-3 groups independently selected from:
   halogen,
   oxo,
   hydroxy, and
   C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, and C₁-C₆ linear and branched alkyl (which may be further substituted with one or two groups independently selected from hydroxy and C₁-C₆ linear and branched alkoxy groups),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino),
C₁-C₆ linear and branched alkyl groups, wherein the alkyl groups are optionally substituted with 1-4 groups independently selected from:
   amino groups optionally substituted with 1-2 groups independently selected from C₁-C₆ linear, branched, and cyclic alkyl (which may be further substituted with 1-2 oxo), and C₁-C₆ linear and branched alkylsulfonyl,
   hydroxy,
   oxo,
   cyano,
   carboxylic acid,
   sulfonic acid,
   —O-heteroaryl,
   carbamate optionally substituted with 1-2 groups independently selected from C₁-C₆ linear and branched alkyl,
   halogen,
   amido optionally substituted with 1-2 groups independently selected from hydroxy, C₁-C₆ linear, branched, and cyclic alkyl groups and C₁-C₆ linear, branched, and cyclic hydroxyalkyl,
   C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
   C₁-C₆ linear and branched alkynyl,
   C₁-C₆ linear and branched alkoxy optionally substituted with 1-2 hydroxy,
   C₁-C₆ linear and branched alkylsulfonyl,
   aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
   carbonyl-(4-methylpiperazin-1-yl),
   carbonyl-(N-morpholino),
   4- to 10-membered heterocyclyl optionally substituted with 1-2 groups independently selected from halogen, oxo, hydroxy, and C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-3 groups independently selected from halogen, oxo, hydroxy, and C₁-C₆ linear, branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl,
halogen,
hydroxy,
oxo,
C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
amide optionally substituted with 1-2 groups independently selected from C₁-C₆ linear and branched alkyl,
carbamate optionally substituted with 1-2 groups independently selected from C₁-C₆ linear and branched alkyl,
carboxamide optionally substituted with 1-2 groups independently selected from C₁-C₆ linear and branched alkyl,
C₁-C₆ linear and branched alkoxy groups optionally substituted with 1-2 groups independently selected from C₁-C₆ linear, branched, and cyclic alkyl, and heterocyclyl,
4- to 10-membered heterocyclyl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups).

In one aspect of the disclosure, the at least one entity (e.g., the at least one compound, deuterated derivative, or pharmaceutically acceptable salt) is chosen from Compounds 1 to 527, deuterated derivatives of those compounds, and pharmaceutically acceptable salts of any of the foregoing.

In some embodiments, the disclosure provides pharmaceutical compositions comprising a compound of Formula I, deuterated derivatives thereof, or a pharmaceutically acceptable salt of any of the foregoing. In some embodiments, the pharmaceutical compositions may comprise at least one compound, deuterated derivative, or pharmaceutically acceptable salt chosen from Compounds 1 to 527, deuterated derivatives of those compounds, and pharmaceutically acceptable salts of any of the foregoing. These compositions may further include at least one additional active pharmaceutical ingredient and/or at least one carrier.

Another aspect of the disclosure provides methods of treating FSGS and/or NDKD comprising administering to a subject in need thereof, at least one compound, deuterated derivative, or pharmaceutically acceptable salt chosen from compounds of Formula I, deuterated derivatives of those compounds, and pharmaceutically acceptable salts of any of the foregoing or a pharmaceutical composition comprising the compound, deuterated derivative, or pharmaceutically acceptable salt. In some embodiments, the methods comprise administering at least one compound, deuterated derivative, or pharmaceutically acceptable salt chosen from Compounds 1 to 527, deuterated derivatives of those compounds, and pharmaceutically acceptable salts of any of the foregoing or a pharmaceutical composition comprising the compound, deuterated derivative, or pharmaceutically acceptable salt.

In some embodiments, the methods of treatment include administration of at least one additional active agent to the subject in need thereof, either in the same pharmaceutical composition as the at least one compound, deuterated derivative, or pharmaceutically acceptable salt chosen from compounds of Formula I, deuterated derivatives of those compounds, and pharmaceutically acceptable salts of any of the foregoing. Alternatively, the additional active agent and the compound, deuterated derivative, or pharmaceutically acceptable salt may be administered as separate pharmaceutical compositions. In some embodiments, the methods comprise administering at least one compound, deuterated derivative, or pharmaceutically acceptable salt chosen from Compounds 1 to 527, deuterated derivatives of those compounds, and pharmaceutically acceptable salts of any of the foregoing with at least one additional active agent either in the same pharmaceutical composition or in separate pharmaceutical compositions.

Also provided herein are methods of inhibiting APOL1, comprising administering to a subject in need thereof, at least one compound, deuterated derivative, or pharmaceutically acceptable salt chosen from compounds of Formula I, deuterated derivatives thereof, and pharmaceutically acceptable salts of any of the foregoing or a pharmaceutical composition comprising the compound, deuterated derivative, or pharmaceutically acceptable salt. In some embodiments, the methods of inhibiting APOL1 comprise administering at least one compound, deuterated derivative, or pharmaceutically acceptable salt chosen from Compounds 1 to 527, deuterated derivatives thereof, and pharmaceutically acceptable salts of any of the foregoing or a pharmaceutical composition comprising the compound, deuterated derivative, or pharmaceutically acceptable salt.

### BRIEF DESCRIPTION OF THE DRAWINGS

FIG. 1 shows the plate map for assay ready plates for dose response (10 point dose response, 100 μM highest final assay, concentration in 20 μL, 2.5-fold serial dilution with total DMSO volume of 200 nL).

### DEFINITIONS

The term “APOL1,” as used herein, means apolipoprotein L1 protein, and the term “APOL1” means apolipoprotein L1 gene.

As used herein, the term “APOL1 mediated kidney disease” refers to a disease or condition that impairs kidney function and can be attributed to APOL1. In some embodiments, APOL1 mediated kidney disease is associated with patients having two APOL1 risk alleles, e.g., patients who are homozygous or compound heterozygous for the G1 or G2 alleles. In some embodiments, the APOL1 mediated kidney disease is chosen from ESKD, NDKD, FSGS, HIV-associated nephropathy, arterionephrosclerosis, lupus nephritis, microalbuminuria, and chronic kidney disease.

The term “FSGS,” as used herein, means focal segmental glomerulosclerosis, which is a disease of the podocyte (glomerular visceral epithelial cells) responsible for proteinuria and progressive decline in kidney function. In some embodiments, FSGS is associated with two APOL1 risk alleles.

The term “NDKD,” as used herein, means non-diabetic kidney disease, which is characterized by severe hypertension and progressive decline in kidney function. In some embodiments, NDKD is associated with two APOL1 risk alleles.

The terms “ESKD” and “ESRD” are used interchangeably herein to refer to end stage kidney disease or end stage renal disease. ESKD/ESRD is the last stage of kidney disease, i.e., kidney failure, and means that the kidneys have stopped working well enough for the patient to survive without dialysis or a kidney transplant. In some embodiments, ESKD/ESRD is associated with two APOL1 risk alleles.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of this disclosure.

Unless otherwise indicated, structures depicted herein are also meant to include all isomeric forms of the structure, e.g., racemic mixtures, cis/trans isomers, geometric (or conformational) isomers, such as (Z) and (F) double bond isomers, and (Z) and (F) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer,” as used herein, refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives described herein. Thus, unless otherwise stated, when a reference is made to a “deuterated derivative” of compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium), at least 4500 (67.5% deuterium incorporation), at least 5000 (75% deuterium incorporation) at least 5500 (82.5% deuterium incorporation), at least 6000 (90% deuterium incorporation), at least 6333.3 (95% deuterium incorporation), at least 6466.7 (97% deuterium incorporation), or at least 6600 (99% deuterium incorporation).

The term “isotopic enrichment factor” as used herein means the ratio between the isotopic abundance and the natural abundance of a specified isotope.

The term “alkyl” or “aliphatic” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic that has a single point of attachment to the rest of the molecule. Unless otherwise specified, alkyl groups contain 1 to 20 carbon atoms. In some embodiments, alkyl groups contain 1 to 10 carbon atoms. In some embodiments, alkyl groups contain 1 to 8 carbon atoms. In some embodiments, alkyl groups contain 1 to 6 carbon atoms, and in some embodiments, alkyl groups contain 1 to 4 carbon atoms, and in yet other embodiments alkyl groups contain 1 to 3 carbon atoms. Non-limiting examples of alkyl groups include, but are not limited to, linear or branched, and substituted or unsubstituted alkyl. In some embodiments, alkyl groups are substituted. In some embodiments, alkyl groups are unsubstituted. In some embodiments, alkyl groups are straight-chain. In some embodiments, alkyl groups are branched.

The terms “cycloalkyl,” “carbocycle,” or “cyclic alkyl” refer to a fused, spirocyclic, or monocyclic C₃₋₈ hydrocarbon or a spirocyclic, bicyclic, bridged bicyclic, tricyclic, or bridged tricyclic C₄₋₁₄ hydrocarbon that is completely saturated or that contains one or more units of unsaturation, but which is not aromatic, wherein any individual ring in said bicyclic ring system has 3 to 7 members. Suitable cycloalkyl groups include, but are not limited to, cycloalkyl, bicyclic cycloalkyl (e.g., decalin), bridged bicycloalkyl such as norbornyl or [2.2.2]bicyclo-octyl, or bridged tricyclic such as adamantyl. In some embodiments, cycloalkyl groups are substituted. In some embodiments, cycloalkyl groups are unsubstituted.

The term “heteroalkyl,” as used herein, means aliphatic groups wherein one, two, or three carbon atoms are independently replaced by a non-carbon atom. In some embodiments, the heteroaryl contains one or more of oxygen, sulfur, and/or nitrogen. In some embodiments, one or more carbon atoms may be replaced by phosphorus, boron, and/or silicon. Heteroalkyl groups may be substituted or unsubstituted, branched or unbranched, cyclic or acyclic, and include “heterocycle,” “heterocyclyl,” or “heterocyclic” groups. In some embodiments, the heteroalkyl is an aminoalkyl. In some embodiments, the heteroalkyl is a thioalkyl. In some embodiments, the heteroalkyl is an alkoxy. In some embodiments, the heteroalkyl has a combination of two or more heteroatoms independently selected from oxygen, nitrogen, phosphorus, and sulfur. In some embodiments, one, two, or three carbon atoms are replaced by nitrogen.

The term “alkenyl,” as used herein, means a straight-chain (i.e., unbranched), branched, substituted or unsubstituted hydrocarbon chain that contains one or more units of saturation, or a monocyclic hydrocarbon or bicyclic hydrocarbon that contains one or more units of unsaturation, but which is not aromatic (referred to herein as, “cyclic alkenyl”). In some embodiments, alkenyl groups are substituted. In some embodiments, alkenyl groups are unsubstituted. In some embodiments, alkenyl groups are straight-chain. In some embodiments, alkenyl groups are branched.

The term “heterocycle,” “heterocyclyl,” or “heterocyclic,” as used herein, means non-aromatic, monocyclic, bicyclic, or tricyclic ring systems in which one or more ring members is an independently chosen heteroatom. In some embodiments, the “heterocycle”, “heterocyclyl”, or “heterocyclic” group has 3 to 14 ring members in which one or more ring members is a heteroatom independently chosen from oxygen, sulfur, nitrogen, phosphorus, silicon, and boron. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one to three heteroatoms independently chosen from oxygen, sulfur, nitrogen, and phosphorus. In some embodiments, the heterocycle has one heteroatom that is a nitrogen atom. In some embodiments, the heterocycle has one heteroatom that is an oxygen atom. In some embodiments, the heterocycle has one heteroatom that is a sulfur atom. In some embodiments, the heterocycle has two heteroatoms that are each independently selected from nitrogen, sulfur, and oxygen. In some embodiments, the heterocycle has three heteroatoms that are each independently selected from nitrogen and oxygen. In some embodiments, heterocycles are substituted. In some embodiments, heterocycles are unsubstituted.

The term “heteroatom” refers to a non-carbon atom. In some embodiments, the heteroatom is selected from oxygen, sulfur, nitrogen, phosphorus, boron, and silicon (including any oxidized form of nitrogen, sulfur, phosphorus, or silicon; the quaternized form of any basic nitrogen or a substitutable nitrogen of a heterocyclic ring, for example, N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units or degrees of unsaturation. Unsaturation is the state in which not all of the available valance bonds in a compound are satisfied by substituents and thus the compound contains double or triple bonds.

The term “alkoxy” or “thioalkyl”, as used herein, refers to an alkyl group, as previously defined, wherein one carbon of the alkyl group is replaced by an oxygen (“alkoxy”) or sulfur (“thioalkyl”) atom, respectively. In some embodiments, one of the two carbon atoms that the oxygen or sulfur atom is linked between is not part of the alkoxy or thioalkyl groups, such as, e.g., when an “alkoxy” group is methoxy, ethoxy, or the like. A “cyclic alkoxy” refers to a monocyclic, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl. In some embodiments, “alkoxy” and/or “thioalkyl” groups are substituted. In some embodiments, “alkoxy” and/or “thioalkyl” groups are unsubstituted.

The terms “haloalkyl” and “haloalkoxy,” as used herein, means a linear or branched alkyl or alkoxy, as the case may be, which is substituted with one or more halogen atoms. Non-limiting examples of haloalkyl groups include —CHF₂, —CH₂F, —CF₃, —CF₂—, and perhaloalkyls, such as —CF₂CF₃. Non-limiting examples of haloalkoxy groups include —OCHF₂, —OCH₂F, —OCF₃, and —OCF₂—.

In some embodiments, the term “hydroxyalkyl” means a linear, branched, or cyclic alkyl which is substituted with one or more hydroxy groups.

The term “halogen” includes F, Cl, Br, and I, i.e., fluoro, chloro, bromo, and iodo, respectively.

The term “aminoalkyl” means an alkyl group which is substituted with or contains an amino group. An aminoalkyl group may be linear or branched.

As used herein, the term “alkylsulfonyl” refers to an alkyl group, as previously defined, wherein one carbon atom of the alkyl group, and the carbon atom's substituents, are replaced by a sulfur atom, and wherein the sulfur atom is further substituted with two oxo groups. An alkylsulfonyl group may be linear or branched. In some embodiments, alkylsulfonyl groups are substituted at the alkyl portion of the alkylsulfonyl group. In some embodiments, alkylsulfonyl groups are unsubstituted at the alkyl portion of the alkylsulfonyl group.

As used herein, an “amino” refers to a group which is a primary, secondary, or tertiary amine.

As used herein, a “carbonyl” group refers to C═O.

As used herein, a “cyano” or “nitrile” group refer to —C≡N.

As used herein, a “hydroxy” group refers to —OH.

As used herein, a “thiol” group refers to —SH.

As used herein, “tert” and “t-” each refer to tertiary.

As used herein, “Me” refers to methyl.

As used herein, an “amido” group refers to a carbonyl group, as previously defined, wherein the carbon of the carbonyl is bonded to an amino group, as previously defined. When a chemical group is said to be substituted by an amido group, that chemical group may be bonded to the carbonyl carbon or to the amino nitrogen of the amido group.

As used herein, a “carbamate” group refers to a carbonyl group, as previously defined, wherein the carbon of the carbonyl group is bonded to an amino group, as previously defined, and a divalent oxygen. When a chemical group is said to be substituted by a carbamate group, that chemical group may be bonded to the divalent oxygen or to the amino nitrogen of the carbamate group.

As used herein, “aromatic groups” or “aromatic rings” refer to chemical groups that contain conjugated, planar ring systems with delocalized pi electron orbitals comprised of [4n+2] p orbital electrons, wherein n is an integer ranging from 0 to 6. Non-limiting examples of aromatic groups include aryl and heteroaryl groups.

The term “aryl,” used alone or as part of a larger moiety as in “arylalkyl,” “arylalkoxy,” or “aryloxyalkyl,” refers to monocyclic, bicyclic, and tricyclic ring systems having a total of five to fourteen ring members, wherein at least one ring in the system is aromatic and wherein each ring in a bicyclic or tricyclic ring system contains 3 to 7 ring members. The term “aryl” also refers to heteroaryl ring systems as defined herein below. Non-limiting examples of aryl groups include phenyl rings. In some embodiments, aryl groups are substituted. In some embodiments, aryl groups are unsubstituted.

The term “heteroaryl,” used alone or as part of a larger moiety as in “heteroarylalkyl” or “heteroarylalkoxy,” refers to monocyclic, bicyclic, and tricyclic ring systems having a total of five to fourteen ring members, wherein at least one ring in the system is aromatic, at least one ring in the system contains one or more heteroatoms, and wherein each ring in a bicyclic or tricyclic ring system contains 3 to 7 ring members. In some embodiments, heteroaryl groups are substituted. In some embodiments, heteroaryl groups have one or more heteroatoms chosen from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, heteroaryl groups are unsubstituted.

Non-limiting examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc), benzyl carbamate (Cbz), acetamide, trifluoroacetamide, triphenylmethylamine, benzylideneamine, and p-toluenesulfonamide. Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999) and 4^{th} Edition (John Wiley & Sons, New Jersey, 2014).

Non-limiting examples of suitable solvents that may be used in this disclosure include, but are not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include, but are not limited to, 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C1-4alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

The terms “patient” and “subject” are used interchangeably and refer to an animal, including a human.

The terms “effective dose” and “effective amount” are used interchangeably herein and refer to that amount of compound that produces the desired effect for which it is administered (e.g., improvement in symptoms of FSGS and/or NDKD, lessening the severity of FSGS and/NDKD or a symptom of FSGS and/or NDKD, and/or reducing progression of FSGS and/or NDKD or a symptom of FSGS and/or NDKD). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treatment” and its cognates refer to slowing or stopping disease progression. “Treatment” and its cognates as used herein, include, but are not limited to the following: lessening the severity of a disease symptom, complete or partial remission, lower risk of kidney failure (e.g., ESRD), and disease-related complications (e.g., edema, susceptibility to infections, or thrombo-embolic events). Improvements in or lessening the severity of one or more symptoms can be readily assessed according to methods and techniques known in the art or subsequently developed. In some embodiments, the terms “treat,” “treating,” and “treatment,” refer to the lessening of severity of one or more symptoms of FSGS and/or NDKD. In some embodiments, “treatment” and its cognates refers to a reduction of the risk of ESRD.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. In some embodiments, the term “about” refers to a value±10%, ±8%, ±6%, ±5%, ±4%, ±2%, or ±1% of a referenced value.

As used herein, the term “ambient conditions” means room temperature, open air, and uncontrolled humidity conditions.

The terms “selected from” and “chosen from” are used interchangeably herein.

The compound of Formula I, I′, II, or II′, a deuterated derivative thereof, or a pharmaceutically acceptable salt of any of the foregoing may be administered once daily, twice daily, or three times daily, for example, for the treatment of FSGS. In some embodiments, the compound of Formula I, I′, II, or II′, deuterated derivative thereof, or pharmaceutically acceptable salt of any of the foregoing is chosen from Compounds 1 to 527, deuterated derivatives thereof, and pharmaceutically acceptable salts of any of the foregoing. In some embodiments, at least one compound of Formula I, I′, II, or II′, deuterated derivative thereof, or pharmaceutically acceptable salt of any of the foregoing is administered once daily. In some embodiments, at least one compound, deuterated derivative, or pharmaceutically acceptable salt chosen from Compounds 1 to 527, deuterated derivatives thereof, and pharmaceutically acceptable salts of any of the foregoing is administered once daily. In some embodiments, at least compound of Formula I, I′, II, or II′, deuterated derivative thereof, or pharmaceutically acceptable salt of any of the foregoing is administered twice daily. In some embodiments, at least one compound, deuterated derivative, or pharmaceutically acceptable salt chosen from Compounds 1 to 527, deuterated derivatives thereof, and pharmaceutically acceptable salts of any of the foregoing is administered twice daily. In some embodiments, at least one compound of Formula I, I′, II, or II′, deuterated derivative thereof, or pharmaceutically acceptable salt of any of the foregoing is administered three times daily. In some embodiments, at least one compound, deuterated derivative, or pharmaceutically acceptable salt chosen from Compounds 1 to 527, deuterated derivatives thereof, and pharmaceutically acceptable salts of any of the foregoing is administered three times daily.

In some embodiments, 2 mg to 1500 mg, 5 mg to 1000 mg, 10 mg to 500 mg, 20 mg to 300 mg, 20 mg to 200 mg, 30 mg to 150 mg, 50 mg to 150 mg, 60 mg to 125 mg, or 70 mg to 120 mg, 80 mg to 115 mg, 90 mg to 110 mg, 95 mg to 110 mg, or 100 mg to 105 mg of the at least one compound, deuterated derivative, or pharmaceutically acceptable salt chosen from compounds of Formula I, I′, II, or II′, deuterated derivatives thereof, and pharmaceutically acceptable salts of any of the foregoing is administered once daily, twice daily, or three times daily. In some embodiments, 2 mg to 1500 mg, 5 mg to 1000 mg, 10 mg to 500 mg, 20 mg to 300 mg, 20 mg to 200 mg, 30 mg to 150 mg, 50 mg to 150 mg, 60 mg to 125 mg, or 70 mg to 120 mg, 80 mg to 115 mg, 90 mg to 110 mg, 95 mg to 110 mg, or 100 mg to 105 mg of the at least one compound, deuterated derivative, or pharmaceutically acceptable salt chosen from Compounds 1 to 527, deuterated derivatives thereof, and pharmaceutically acceptable salts of any of the foregoing is administered once daily, twice daily, or three times daily.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, pharmaceutically acceptable salts, solvates, and deuterated derivatives disclosed herein are based upon the free base form of the reference compound. For example, “10 mg of at least one compound chosen from compounds of Formula I, . . . and pharmaceutically acceptable salts thereof” includes 10 mg of a compound of Formula I, and a concentration of a pharmaceutically acceptable salt of that compound of Formula I that is equivalent to 10 mg of that compound of Formula I.

### Compounds and Compositions

In some embodiments of the disclosure, the compound, deuterated derivative, or pharmaceutically acceptable salt for treating APOL1 mediated diseases, such as FSGS and/or NDKD, is selected from compounds of Formula I:

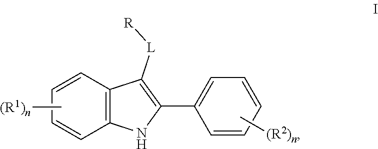

deuterated derivatives thereof, and pharmaceutically acceptable salts of any of the foregoing, wherein:
(i) R is selected from —C(O)NR³R⁴, —NR⁵C(O)R³, —NR⁵C(O)NR³R⁴, —NR³R⁴, —OR³, —NR⁵—SO₂R³, —OC(O)NR³R⁴, —C(O)OR³,

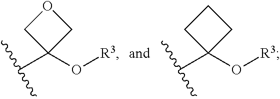

(ii) L is selected from divalent C₁-C₆ linear and branched alkyl (e.g., divalent C₁-C₆ linear and C₃-C₆ branched alkyl), divalent C₂-C₆ linear and branched alkenyl (e.g., divalent C₂-C₆ linear and C₃-C₆ branched alkenyl), divalent C₂-C₆ linear and branched alkynyl (e.g., divalent C₂-C₆ linear and C₃-C₆ branched alkynyl), and divalent 1- to 7-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;
   (iii) each R¹ is independently selected from:
halogen,
hydroxy,
thiol,
amino,
cyano,
C₁-C₆ linear, branched, and cyclic alkyl (e.g., C₁-C₆ linear, C₃-C₆ branched, and C₃-C₆ cyclic alkyl) optionally substituted with 1-3 groups independently selected from hydroxy and halogen,
C₂-C₆ linear, branched, and cyclic alkenyl (e.g., C₂-C₆ linear, C₃-C₆ branched, and C₃-C₆ cyclic alkenyl),
C₁-C₆ linear, branched, and cyclic alkoxy (e.g., C₁-C₆ linear, C₂-C₆ branched, and C₂-C₆ cyclic alkoxy) optionally substituted with 1-3 groups independently selected from halogen,
C₁-C₆ linear, branched, and cyclic thioalkyl (e.g., C₁-C₆ linear, C₂-C₆ branched, and C₂-C₆ cyclic thioalkyl) optionally substituted with 1-3 groups independently selected from halogen, and
C₁-C₆ linear, branched, and cyclic aminoalkyl optionally substituted with 1-3 groups independently selected from halogen,
or two R¹ groups, together with the carbon atoms to which they are attached, may form a C₄-C₈ cycloalkyl, aryl, or heteroaryl;
   (iv) each R² is independently selected from:
halogen,
hydroxy,
thiol,
amino,
cyano,
C₁-C₆ linear, branched, and cyclic alkyl (e.g., C₁-C₆ linear, C₃-C₆ branched, and C₃-C₆ cyclic alkyl) optionally substituted with 1-3 groups independently selected from hydroxy and halogen,
C₂-C₄ linear, branched, and cyclic alkenyl (e.g., C₂-C₄ linear, C₃-C₄ branched, and C₃-C₄ cyclic alkenyl),
C₁-C₆ linear, branched, and cyclic alkoxy (e.g., C₁-C₆ linear, C₂-C₆ branched, and C₂-C₆ cyclic alkoxy) optionally substituted with 1-3 groups independently selected from halogen,
C₁-C₄ linear, branched, and cyclic thioalkyl (e.g., C₁-C₄ linear, C₂-C₄ branched, and C₂-C₄ cyclic thioalkyl) optionally substituted with 1-3 groups independently selected from halogen, and
C₁-C₄ linear, branched, and cyclic aminoalkyl optionally substituted with 1-3 groups independently selected from halogen,
   (v) each n is independently selected from 0, 1, 2, 3, and 4;
   (vi) R³ and R⁴ are independently selected from:
hydrogen,
C₁-C₆ linear and branched alkylsulfonyl (e.g., C₁-C₆ linear and C₂-C₆ branched alkyl sulfonyl),
C₂-C₆ linear and branched alkenyl (e.g., C₂-C₆ linear and C₃-C₆ branched alkenyl),
amino optionally substituted with 1-2 groups independently selected from C₁-C₆ linear and branched alkyl (e.g., C₁-C₆ linear and C₃-C₆ branched alkyl) and C₃-C₆ cycloalkyl,
amide optionally substituted with 1-2 groups independently selected from C₁-C₃ alkyl,
C₁-C₆ linear and branched alkoxy (e.g., C₁-C₆ linear and C₂-C₆ branched alkoxy) optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl (e.g., C₃-C₆ cyclic alkyl) optionally substituted with 1-2 groups independently selected from:
   halogen,
   hydroxy,
   oxo,
   amino optionally substituted with 1-2 groups independently selected from hydrogen and C₁-C₆ linear or branched alkyl (e.g., C₁-C₆ linear or C₃-C₆ branched alkyl),
   aryl optionally substituted with 1-2 groups independently selected from halogen,
   C₁-C₆ linear and branched alkyl groups (e.g., C₁-C₆ linear and C₃-C₆ branched alkyl groups) (which may be further substituted with 1-3 groups independently selected from hydroxy, oxo, halogen, and C₁-C₆ linear and branched alkoxy groups (e.g., C₁-C₆ linear and C₂-C₆ branched alkoxy groups)),
   carbamate optionally substituted with 1-2 groups independently selected from C₁-C₆ linear and branched alkyl (e.g., C₁-C₆ linear and C₃-C₆ branched alkyl),
   C₁-C₆ linear and branched alkoxy (e.g., C₁-C₆ linear and C₂-C₆ branched alkoxy), and
   amide,
4- to 10-membered heterocyclyl optionally substituted with 1-3 groups independently selected from:
   halogen,
   oxo,
   hydroxy,
   amino, and
   C₁-C₆ linear and branched alkyl (e.g., C₁-C₆ linear and C₃-C₆ branched alkyl) (which may be further substituted with 1-2 groups independently selected from hydroxy, oxo, and C₁-C₆ linear and branched alkoxy (e.g., C₁-C₆ linear and C₂-C₆ branched alkoxy)),
aryl optionally substituted with 1-3 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy groups (e.g., C₁-C₆ linear and C₂-C₆ branched alkoxy groups)),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino), and
C₁-C₆ linear and branched alkyl groups (e.g., C₁-C₆ linear and C₃-C₆ branched alkyl groups), wherein the alkyl groups are optionally substituted with 1-4 groups independently selected from:
   amino groups optionally substituted with 1-2 groups independently selected from hydroxy, C₁-C₆ linear, branched, and cyclic alkyl (e.g., C₁-C₆ linear, C₃-C₆ branched, and C₃-C₆ cyclic alkyl) (which may be further substituted with 1-2 oxo), and C₁-C₆ linear and branched alkylsulfonyl (e.g., C₁-C₆ linear and C₂-C₆ branched alkylsulfonyl),
   hydroxy,
   oxo,
   cyano,
   carboxylic acid,
   sulfonic acid,
   —O-heteroaryl,
   carbamate optionally substituted with 1-2 groups independently selected from C₁-C₆ linear and branched alkyl (e.g., C₁-C₆ linear and C₃-C₆ branched alkyl),
   halogen,
   amido optionally substituted with 1-2 groups independently selected from hydroxy, C₁-C₆ linear, branched, and cyclic alkyl groups (e.g., C₁-C₆ linear, C₃-C₆ branched, and C₃-C₆ cyclic alkyl groups), and C₁-C₆ linear, branched, and cyclic hydroxyalkyl (e.g., C₁-C₆ linear, C₃-C₆ branched, and C₃-C₆ cyclic hydroxyalkyl groups),
   C₃-C₆ cyclic alkyl optionally substituted with 1-2 groups independently selected from halogen, hydroxy, C₁-C₆ linear and branched hydroxyalkyl (e.g., C₁-C₆ linear and C₃-C₆ branched hydroxyalkyl), C₁-C₆ linear and branched alkoxy (e.g., C₁-C₆ linear and C₂-C₆ branched alkoxy), and carbamate (which may be further substituted with 1-2 groups independently selected from C₁-C₆ linear and branched alkyl (e.g., C₁-C₆ linear and C₃-C₆ branched alkyl)),
   C₁-C₆ linear and branched alkynyl (e.g., C₂-C₆ linear and branched alkynyl, e.g., C₂-C₆ linear and C₃-C₆ branched alkynyl),
   C₁-C₆ linear and branched alkoxy (e.g., C₁-C₆ linear and C₂-C₆ branched alkoxy) optionally substituted with 1-2 hydroxy,
   C₁-C₆ linear and branched alkylsulfonyl (e.g., C₁-C₆ linear and C₂-C₆ branched alkylsulfonyl),
   aryl optionally substituted with 1-2 groups independently selected from halogen groups, hydroxy, and C₁-C₆ linear and branched alkyl groups (e.g., C₁-C₆ linear and C₃-C₆ branched alkyl groups) (which may be further substituted with 1-2 groups independently selected from hydroxy and C₁-C₆ linear and branched alkoxy groups (e.g., C₁-C₆ linear and C₂-C₆ branched alkoxy groups)),
   carbonyl-(4-methylpiperazin-1-yl),
   carbonyl-(N-morpholino),
   4- to 10-membered heterocyclyl optionally substituted with 1-3 groups independently selected from halogen, oxo, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy (e.g., C₁-C₆ linear and C₂-C₆ branched alkoxy)), and
   4- to 10-membered heteroaryl optionally substituted with 1-3 groups independently selected from halogen, oxo, hydroxy, and C₁-C₆ linear, branched, and cyclic alkyl (e.g., C₁-C₆ linear, C₃-C₆ branched, and C₃-C₆ cyclic alkyl) (which may be further substituted with 1-3 groups independently selected from halogen, hydroxy, and C₁-C₆ linear and branched alkoxy (e.g., C₁-C₆ linear and C₂-C₆ branched alkoxy)),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl (e.g., C₁-C₆ linear, C₃-C₆ branched, and C₃-C₆ cyclic alkyl groups),
halogen,
hydroxy,
oxo,
C₁-C₆ linear, branched, and cyclic alkyl (e.g., C₁-C₆ linear, C₃-C₆ branched, and C₃-C₆ cyclic alkyl groups) optionally substituted with 1-2 groups independently selected from hydroxy, amino, C₁-C₆ linear, branched, and cyclic alkoxy groups (e.g., C₁-C₆ linear, C₂-C₆ branched, and C₂-C₆ cyclic alkoxy groups), and carbamate (which may be further substituted with 1-2 groups independently selected from C₁-C₆ linear and branched alkyl (e.g., C₁-C₆ linear and C₃-C₆ branched alkyl)),
amide optionally substituted with 1-2 groups independently selected from C₁-C₆ linear and branched alkyl (e.g., C₁-C₆ linear and C₃-C₆ branched alkyl),
carbamate optionally substituted with 1-2 groups independently selected from C₁-C₆ linear and branched alkyl (e.g., C₁-C₆ linear and C₃-C₆ branched alkyl),
carboxamide optionally substituted with 1-2 groups independently selected from C₁-C₆ linear and branched alkyl (e.g., C₁-C₆ linear and C₃-C₆ branched alkyl),
C₁-C₆ linear and branched alkoxy groups (e.g., C₁-C₆ linear and C₂-C₆ branched alkoxy groups) optionally substituted with 1-2 groups independently selected from C₁-C₆ linear, branched, and cyclic alkyl (e.g., C₁-C₆ linear, C₃-C₆ branched, and C₃-C₆ cyclic alkyl) and heterocyclyl,
4- to 10-membered heterocyclyl optionally substituted with 1-2 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy (e.g., C₁-C₆ linear and C₂-C₆ branched alkoxy)), and
4- to 10-membered heteroaryl optionally substituted with 1-2 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy groups (e.g., C₁-C₆ linear and C₂-C₆ branched alkoxy groups));

and

(vii) R⁵ is selected from hydrogen and C₁-C₆ linear or branched alkyl (e.g., C₁-C₆ linear or C₃-C₆ branched alkyl). In certain embodiments, the following compounds are excluded from Formula I:

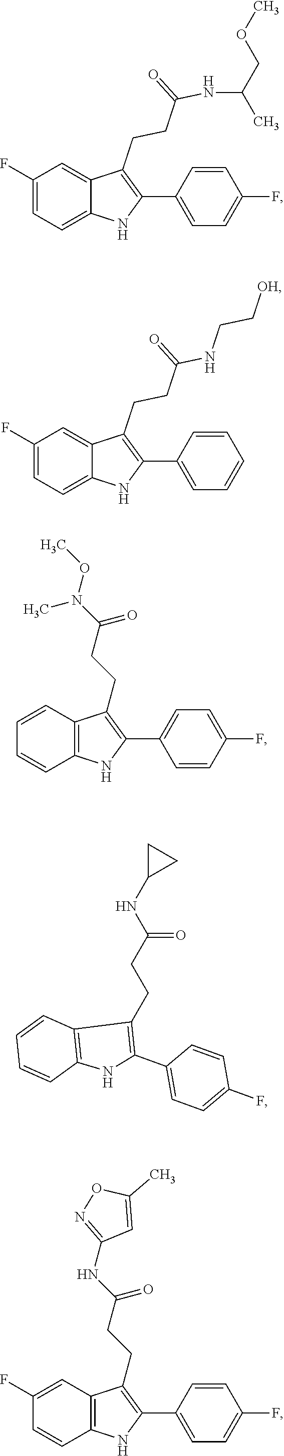 [SMILES: COCC(C)NC(=O)CCc1c(-c2ccc(F)cc2)[nH]c2ccc(F)cc12 ; O=C(CCc1c(-c2ccccc2)[nH]c2ccc(F)cc12)NCCO ; CON(C)C(=O)CCc1c(-c2ccc(F)cc2)[nH]c2ccccc12 ; O=C(CCc1c(-c2ccc(F)cc2)[nH]c2ccccc12)NC1CC1 ; Cc1cc(NC(=O)CCc2c(-c3ccc(F)cc3)[nH]c3ccc(F)cc23)no1]

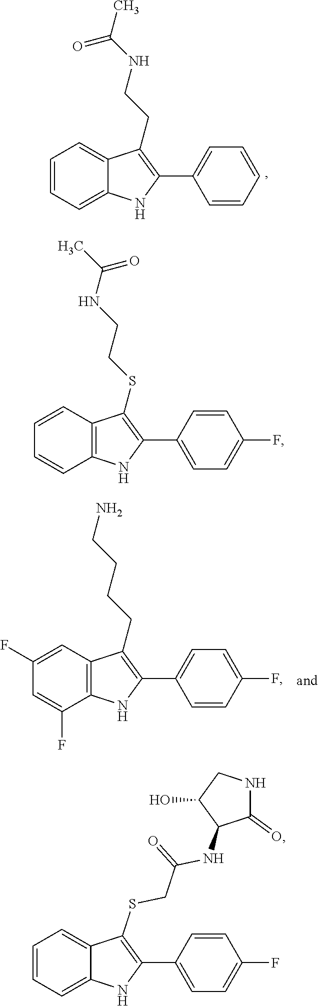

and compounds where -L-R in Formula I is

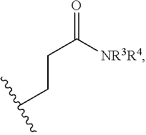

and R³ and R⁴ are

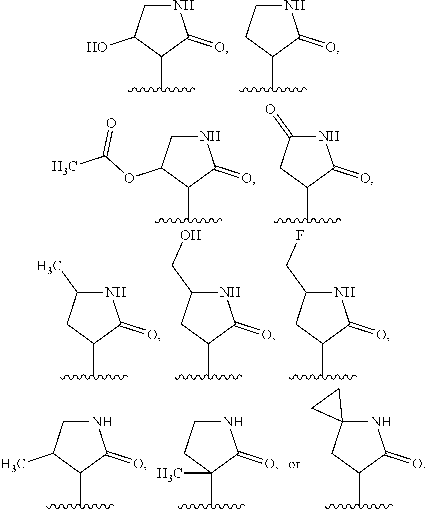

In some embodiments, when L is a divalent C₂ linear alkyl optionally substituted with 1-2 groups independently selected from methyl, halogen, and hydroxy and R is —NR³R⁴, then R³ and R⁴ are not

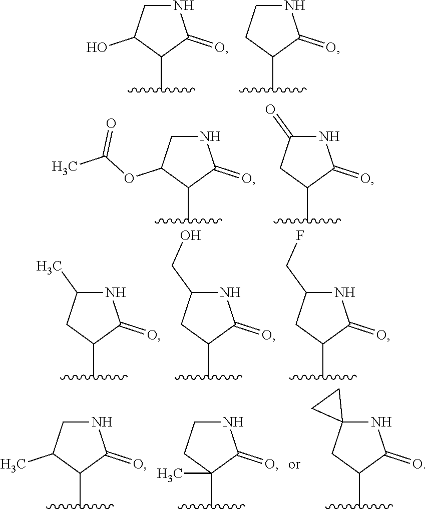

In some embodiments, when L is a divalent C₂ linear alkyl optionally substituted with 1-2 groups independently selected from methyl, halogen, and hydroxy and R is —NR³R⁴, then R³ and R⁴ are not

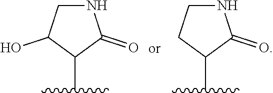

In some embodiments, L is selected from divalent C₁-C₆ linear and branched alkyl, divalent C₂-C₆ linear and branched alkenyl, divalent C₂-C₆ linear and branched alkynyl, and divalent 1- to 6-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;

each R² is independently selected from:
halogen,
hydroxy,
thiol,
amino,
cyano,
C₁-C₄ linear, branched, and cyclic alkyl optionally substituted with 1-3 groups independently selected from hydroxy and halogen,
C₂-C₄ linear, branched, and cyclic alkenyl,
C₁-C₄ linear, branched, and cyclic alkoxy optionally substituted with 1-3 groups independently selected from halogen,
C₁-C₄ linear, branched, and cyclic thioalkyl optionally substituted with 1-3 groups independently selected from halogen, and
C₁-C₄ linear, branched, and cyclic aminoalkyl optionally substituted with 1-3 groups independently selected from halogen; and

R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl and C₃-C₆ cycloalkyl,
amide optionally substituted with 1-2 groups independently selected from C₁-C₃ alkyl, and
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl optionally substituted with 1-2 groups independently selected from:
   halogen,
   hydroxy,
   oxo,
   amino optionally substituted with 1-2 groups independently selected from hydrogen and C₁-C₆ linear or branched alkyl,
   aryl optionally substituted with 1-2 groups independently selected from halogen,
   C₁-C₆ linear and branched alkyl groups (which may be further substituted with 1-3 groups independently selected from hydroxy, oxo, halogen, and C₁-C₆ linear and branched alkoxy groups),
   carbamate optionally substituted with 1-2 groups independently selected from C₁-C₆ linear and branched alkyl,
   C₁-C₆ linear and branched alkoxy, and
   amide,
4- to 10-membered heterocyclyl optionally substituted with 1-3 groups independently selected from:
   halogen,
   oxo,
   hydroxy, and
   C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, and C₁-C₆ linear and branched alkyl (which may be further substituted with one or two groups independently selected from hydroxy and C₁-C₆ linear and branched alkoxy groups),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino),
C₁-C₆ linear and branched alkyl groups, wherein the alkyl groups are optionally substituted with 1-4 groups independently selected from:
   amino groups optionally substituted with 1-2 groups independently selected from C₁-C₆ linear, branched, and cyclic alkyl (which may be further substituted with 1-2 oxo), and C₁-C₆ linear and branched alkylsulfonyl,
   hydroxy,
   oxo,
   cyano,
   carboxylic acid,
   sulfonic acid,
   —O-heteroaryl,
   carbamate optionally substituted with 1-2 groups independently selected from C₁-C₆ linear and branched alkyl,
   halogen,
   amido optionally substituted with 1-2 groups independently selected from hydroxy, C₁-C₆ linear, branched, and cyclic alkyl groups and C₁-C₆ linear, branched, and cyclic hydroxyalkyl,
   C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
   C₁-C₆ linear and branched alkynyl,
   C₁-C₆ linear and branched alkoxy optionally substituted with 1-2 hydroxy,
   C₁-C₆ linear and branched alkylsulfonyl,
   aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
   carbonyl-(4-methylpiperazin-1-yl),
   carbonyl-(N-morpholino),
   4- to 10-membered heterocyclyl optionally substituted with 1-2 groups independently selected from halogen, oxo, hydroxy, and C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-3 groups independently selected from halogen, oxo, hydroxy, and C₁-C₆ linear, branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl,
halogen,
hydroxy,
oxo,
C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
amide optionally substituted with 1-2 groups independently selected from C₁-C₆ linear and branched alkyl,
carbamate optionally substituted with 1-2 groups independently selected from C₁-C₆ linear and branched alkyl,
carboxamide optionally substituted with 1-2 groups independently selected from C₁-C₆ linear and branched alkyl,
C₁-C₆ linear and branched alkoxy groups optionally substituted with 1-2 groups independently selected from C₁-C₆ linear, branched, and cyclic alkyl, and heterocyclyl,
4- to 10-membered heterocyclyl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups).

In some embodiments, R₃ is hydrogen or methyl.

In some embodiments, R₃ is hydrogen.

In some embodiments, each R₁ is independently chosen from halogen groups.

In some embodiments, each R₁ is fluoro.

In some embodiments, each R₂ is independently chosen from halogen groups and methyl.

In some embodiments, each R₂ is independently chosen from halogen groups.

In some embodiments, each R₂ is fluoro.

In some embodiments, each n is 1 or 2.

In some embodiments, each n is 2.

In some embodiments, R₅ is hydrogen.

In some embodiments, the compound of Formula I, deuterated derivative thereof, or pharmaceutically acceptable salt of any of the foregoing is selected from Compounds 1 to 527 depicted in Table 1, deuterated derivatives thereof, and pharmaceutically acceptable salts of any of the foregoing. A wavy line in a compound in Table 1 (i.e., [symbol] ) depicts a bond between two atoms and indicates a position of mixed stereochemistry for a collection of molecules, such as a racemic mixture, cis/trans isomers, or (E)/(Z) isomers.

TABLE 1
Compounds 1 to 527

| | |
|---|---|
| 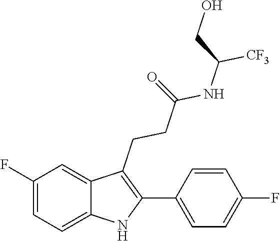 [SMILES: O=C(CCc1c(-c2ccc(F)cc2)[nH]c2ccc(F)cc12)N[C@@H](CO)C(F)(F)F] | 1 |
| 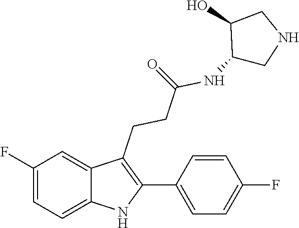 [SMILES: O=C(CCc1c(-c2ccc(F)cc2)[nH]c2ccc(F)cc12)N[C@H]1CNC[C@@H]1O] | 2 |
| 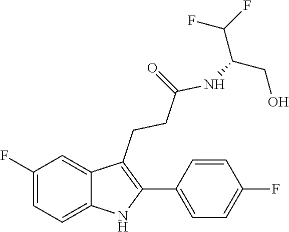 [SMILES: O=C(CCc1c(-c2ccc(F)cc2)[nH]c2ccc(F)cc12)N[C@@H](CO)C(F)F] | 3 |
| 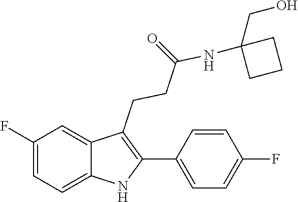 [SMILES: O=C(CCc1c(-c2ccc(F)cc2)[nH]c2ccc(F)cc12)NC1(CO)CCC1] | 4 |
| 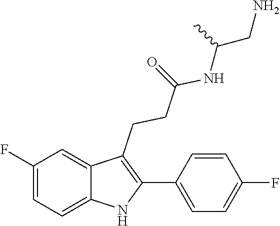 [SMILES: CC(CN)NC(=O)CCc1c(-c2ccc(F)cc2)[nH]c2ccc(F)cc12] | 5 |
| 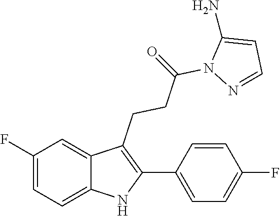 [SMILES: Nc1ccnn1C(=O)CCc1c(-c2ccc(F)cc2)[nH]c2ccc(F)cc12] | 6 |
| 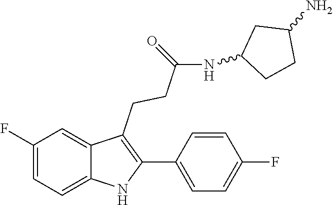 [SMILES: NC1CCC(NC(=O)CCc2c(-c3ccc(F)cc3)[nH]c3ccc(F)cc23)C1] | 7 |
| 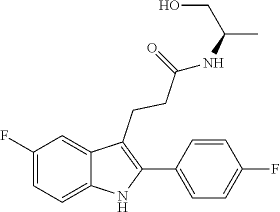 [SMILES: C[C@H](CO)NC(=O)CCc1c(-c2ccc(F)cc2)[nH]c2ccc(F)cc12] | 8 |
| 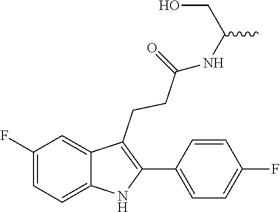 [SMILES: CC(CO)NC(=O)CCc1c(-c2ccc(F)cc2)[nH]c2ccc(F)cc12] | 9 |
| 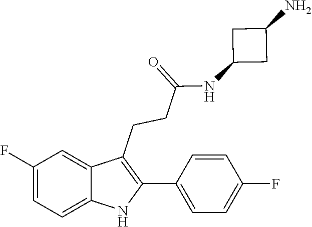 [SMILES: N[C@H]1C[C@@H](NC(=O)CCc2c(-c3ccc(F)cc3)[nH]c3ccc(F)cc23)C1] | 10 |
| 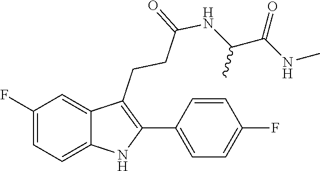 [SMILES: CNC(=O)C(C)NC(=O)CCc1c(-c2ccc(F)cc2)[nH]c2ccc(F)cc12] | 11 |
| 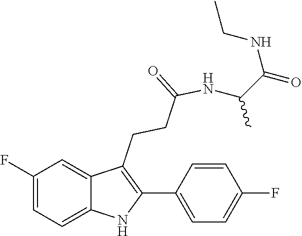 [SMILES: CCNC(=O)C(C)NC(=O)CCc1c(-c2ccc(F)cc2)[nH]c2ccc(F)cc12] | 12 |
| 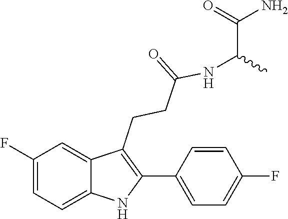 [SMILES: CC(NC(=O)CCc1c(-c2ccc(F)cc2)[nH]c2ccc(F)cc12)C(N)=O] | 13 |
| 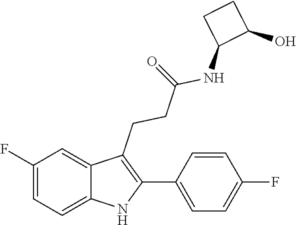 [SMILES: O=C(CCc1c(-c2ccc(F)cc2)[nH]c2ccc(F)cc12)N[C@H]1CC[C@H]1O] | 14 |
| 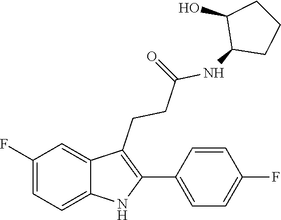 [SMILES: O=C(CCc1c(-c2ccc(F)cc2)[nH]c2ccc(F)cc12)N[C@@H]1CCC[C@@H]1O] | 15 |
| 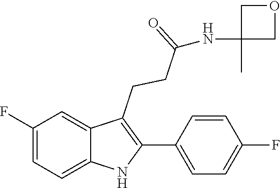 [SMILES: CC1(NC(=O)CCc2c(-c3ccc(F)cc3)[nH]c3ccc(F)cc23)COC1] | 16 |
| 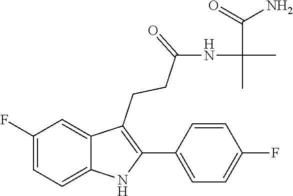 [SMILES: CC(C)(NC(=O)CCc1c(-c2ccc(F)cc2)[nH]c2ccc(F)cc12)C(N)=O] | 17 |
| 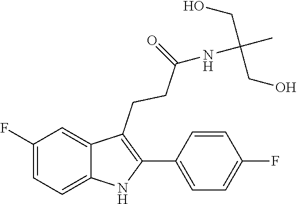 [SMILES: CC(CO)(CO)NC(=O)CCc1c(-c2ccc(F)cc2)[nH]c2ccc(F)cc12] | 18 |
| 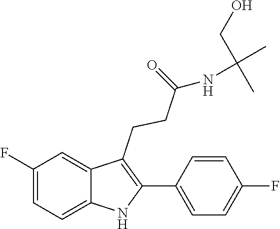 [SMILES: CC(C)(CO)NC(=O)CCc1c(-c2ccc(F)cc2)[nH]c2ccc(F)cc12] | 19 |
| 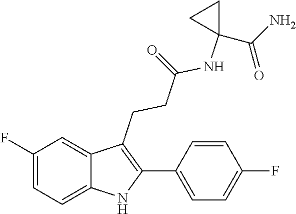 [SMILES: NC(=O)C1(NC(=O)CCc2c(-c3ccc(F)cc3)[nH]c3ccc(F)cc23)CC1] | 20 |
| 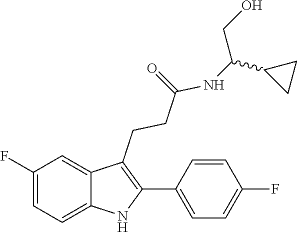 [SMILES: O=C(CCc1c(-c2ccc(F)cc2)[nH]c2ccc(F)cc12)NC(CO)C1CC1] | 21 |
| 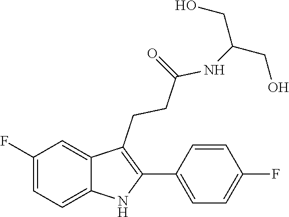 [SMILES: O=C(CCc1c(-c2ccc(F)cc2)[nH]c2ccc(F)cc12)NC(CO)CO] | 22 |
| 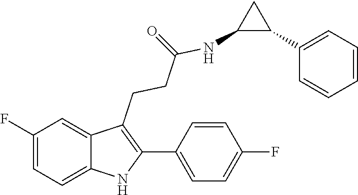 [SMILES: O=C(CCc1c(-c2ccc(F)cc2)[nH]c2ccc(F)cc12)N[C@H]1C[C@@H]1c1ccccc1] | 23 |
| 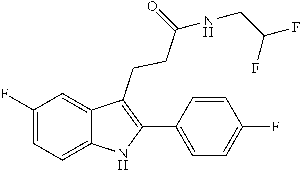 [SMILES: O=C(CCc1c(-c2ccc(F)cc2)[nH]c2ccc(F)cc12)NCC(F)F] | 24 |
| 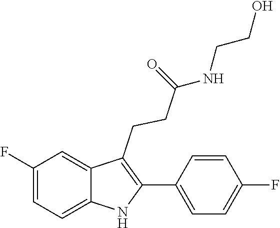 [SMILES: O=C(CCc1c(-c2ccc(F)cc2)[nH]c2ccc(F)cc12)NCCO] | 25 |
| 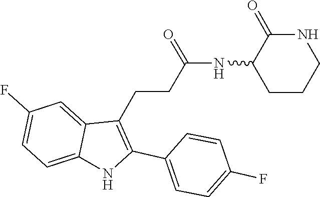 [SMILES: O=C(CCc1c(-c2ccc(F)cc2)[nH]c2ccc(F)cc12)NC1CCCNC1=O] | 26 |
| 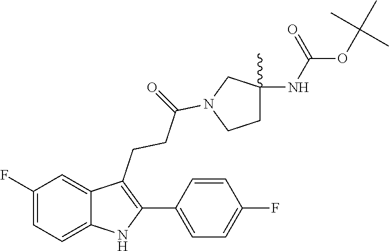 [SMILES: CC1(NC(=O)OC(C)(C)C)CCN(C(=O)CCc2c(-c3ccc(F)cc3)[nH]c3ccc(F)cc23)C1] | 27 |
| 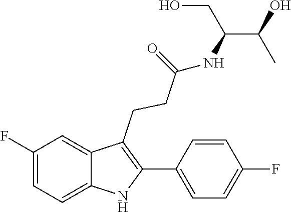 [SMILES: C[C@H](O)[C@H](CO)NC(=O)CCc1c(-c2ccc(F)cc2)[nH]c2ccc(F)cc12] | 28 |
| 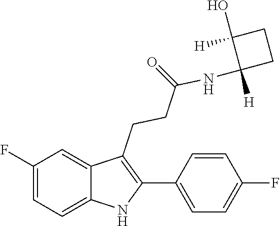 [SMILES: O=C(CCc1c(-c2ccc(F)cc2)[nH]c2ccc(F)cc12)N[C@H]1CC[C@@H]1O] | 29 |
| 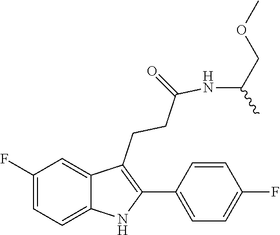 [SMILES: COCC(C)NC(=O)CCc1c(-c2ccc(F)cc2)[nH]c2ccc(F)cc12] | 30 |
| 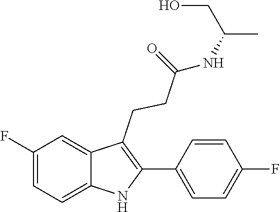 [SMILES: C[C@@H](CO)NC(=O)CCc1c(-c2ccc(F)cc2)[nH]c2ccc(F)cc12] | 31 |
| 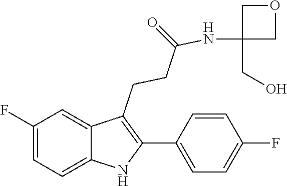 [SMILES: O=C(CCc1c(-c2ccc(F)cc2)[nH]c2ccc(F)cc12)NC1(CO)COC1] | 32 |
| 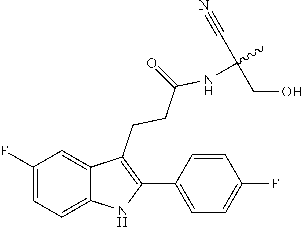 [SMILES: CC(C#N)(CO)NC(=O)CCc1c(-c2ccc(F)cc2)[nH]c2ccc(F)cc12] | 33 |
| 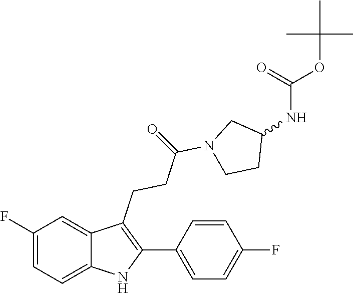 [SMILES: CC(C)(C)OC(=O)NC1CCN(C(=O)CCc2c(-c3ccc(F)cc3)[nH]c3ccc(F)cc23)C1] | 34 |
| 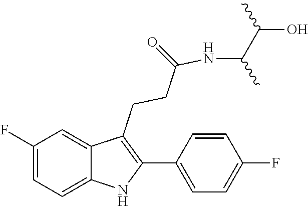 | 35 |
| 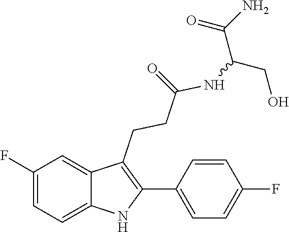 [SMILES: NC(=O)C(CO)NC(=O)CCc1c(-c2ccc(F)cc2)[nH]c2ccc(F)cc12] | 36 |
| 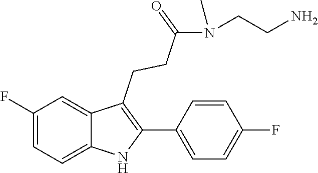 [SMILES: CN(CCN)C(=O)CCc1c(-c2ccc(F)cc2)[nH]c2ccc(F)cc12] | 37 |
| 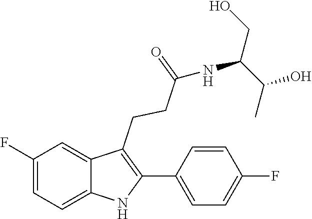 [SMILES: C[C@@H](O)[C@H](CO)NC(=O)CCc1c(-c2ccc(F)cc2)[nH]c2ccc(F)cc12] | 38 |
| 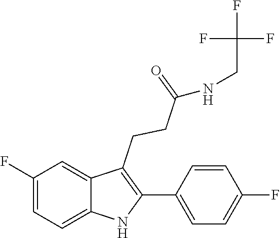 [SMILES: O=C(CCc1c(-c2ccc(F)cc2)[nH]c2ccc(F)cc12)NCC(F)(F)F] | 39 |
| 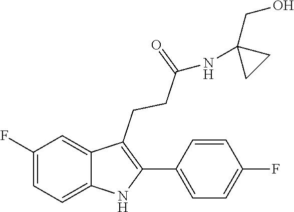 [SMILES: O=C(CCc1c(-c2ccc(F)cc2)[nH]c2ccc(F)cc12)NC1(CO)CC1] | 40 |
| 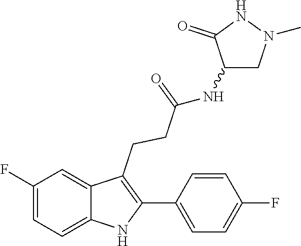 [SMILES: CN1CC(NC(=O)CCc2c(-c3ccc(F)cc3)[nH]c3ccc(F)cc23)C(=O)N1] | 41 |
| 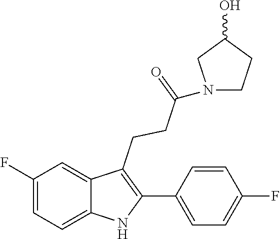 [SMILES: O=C(CCc1c(-c2ccc(F)cc2)[nH]c2ccc(F)cc12)N1CCC(O)C1] | 42 |
| 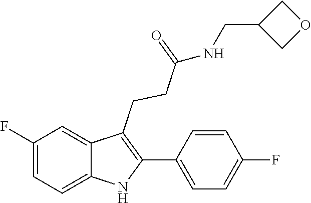 [SMILES: O=C(CCc1c(-c2ccc(F)cc2)[nH]c2ccc(F)cc12)NCC1COC1] | 43 |
| 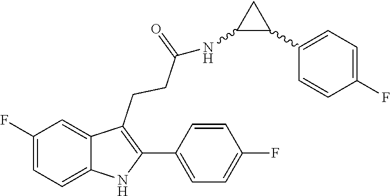 [SMILES: O=C(CCc1c(-c2ccc(F)cc2)[nH]c2ccc(F)cc12)NC1CC1c1ccc(F)cc1] | 44 |
| 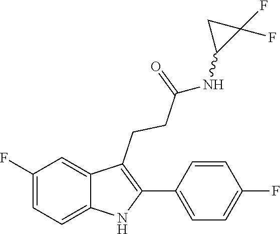 [SMILES: O=C(CCc1c(-c2ccc(F)cc2)[nH]c2ccc(F)cc12)NC1CC1(F)F] | 45 |
| 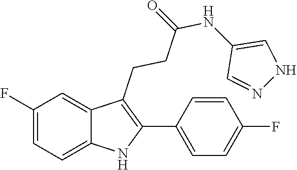 [SMILES: O=C(CCc1c(-c2ccc(F)cc2)[nH]c2ccc(F)cc12)Nc1cn[nH]c1] | 46 |
| 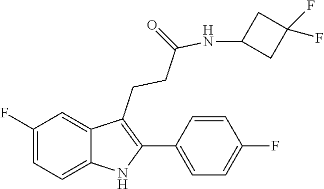 [SMILES: O=C(CCc1c(-c2ccc(F)cc2)[nH]c2ccc(F)cc12)NC1CC(F)(F)C1] | 47 |
| 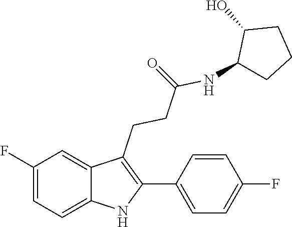 [SMILES: O=C(CCc1c(-c2ccc(F)cc2)[nH]c2ccc(F)cc12)N[C@@H]1CCC[C@H]1O] | 48 |
| 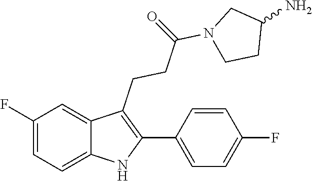 [SMILES: NC1CCN(C(=O)CCc2c(-c3ccc(F)cc3)[nH]c3ccc(F)cc23)C1] | 49 |
| 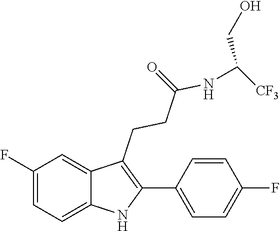 [SMILES: O=C(CCc1c(-c2ccc(F)cc2)[nH]c2ccc(F)cc12)N[C@H](CO)C(F)(F)F] | 50 |
| 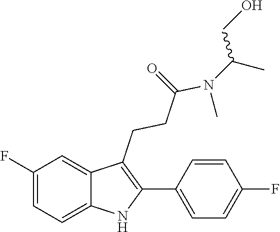 [SMILES: CC(CO)N(C)C(=O)CCc1c(-c2ccc(F)cc2)[nH]c2ccc(F)cc12] | 51 |
| 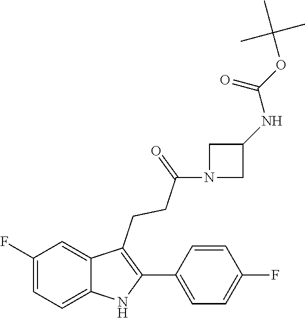 [SMILES: CC(C)(C)OC(=O)NC1CN(C(=O)CCc2c(-c3ccc(F)cc3)[nH]c3ccc(F)cc23)C1] | 52 |
| 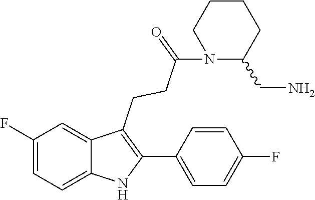 [SMILES: NCC1CCCCN1C(=O)CCc1c(-c2ccc(F)cc2)[nH]c2ccc(F)cc12] | 53 |
| 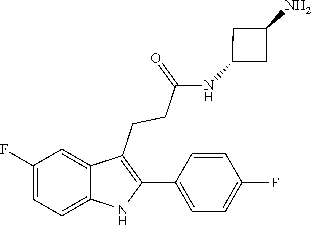 [SMILES: N[C@H]1C[C@H](NC(=O)CCc2c(-c3ccc(F)cc3)[nH]c3ccc(F)cc23)C1] | 54 |
| 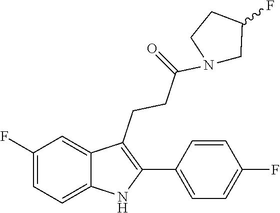 [SMILES: O=C(CCc1c(-c2ccc(F)cc2)[nH]c2ccc(F)cc12)N1CCC(F)C1] | 55 |
| 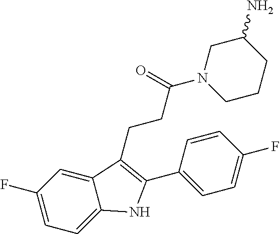 [SMILES: NC1CCCN(C(=O)CCc2c(-c3ccc(F)cc3)[nH]c3ccc(F)cc23)C1] | 56 |
| 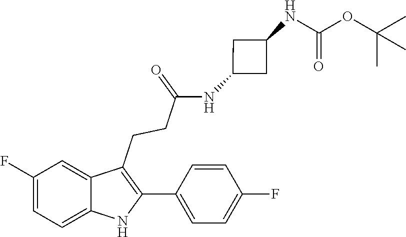 [SMILES: CC(C)(C)OC(=O)N[C@H]1C[C@H](NC(=O)CCc2c(-c3ccc(F)cc3)[nH]c3ccc(F)cc23)C1] | 57 |
| 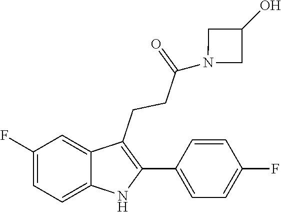 [SMILES: O=C(CCc1c(-c2ccc(F)cc2)[nH]c2ccc(F)cc12)N1CC(O)C1] | 58 |
| 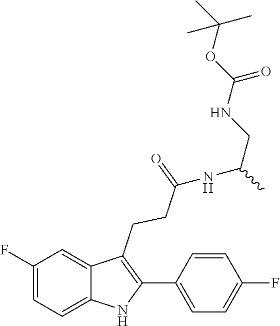 [SMILES: CC(CNC(=O)OC(C)(C)C)NC(=O)CCc1c(-c2ccc(F)cc2)[nH]c2ccc(F)cc12] | 59 |
| 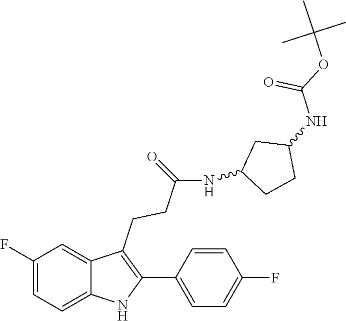 [SMILES: CC(C)(C)OC(=O)NC1CCC(NC(=O)CCc2c(-c3ccc(F)cc3)[nH]c3ccc(F)cc23)C1] | 60 |
| 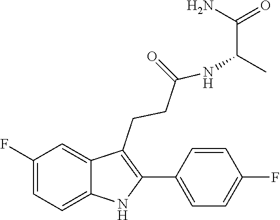 [SMILES: C[C@H](NC(=O)CCc1c(-c2ccc(F)cc2)[nH]c2ccc(F)cc12)C(N)=O] | 61 |
| 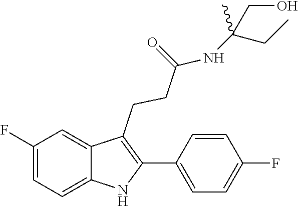 [SMILES: CCC(C)(CO)NC(=O)CCc1c(-c2ccc(F)cc2)[nH]c2ccc(F)cc12] | 62 |
| 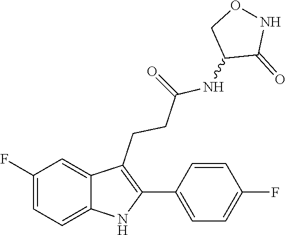 [SMILES: O=C(CCc1c(-c2ccc(F)cc2)[nH]c2ccc(F)cc12)NC1CONC1=O] | 63 |
| 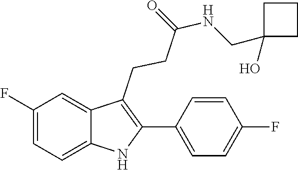 [SMILES: O=C(CCc1c(-c2ccc(F)cc2)[nH]c2ccc(F)cc12)NCC1(O)CCC1] | 64 |
| 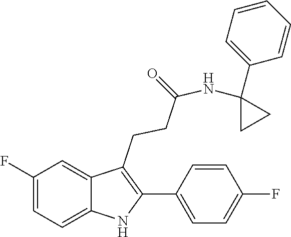 [SMILES: O=C(CCc1c(-c2ccc(F)cc2)[nH]c2ccc(F)cc12)NC1(c2ccccc2)CC1] | 65 |
| 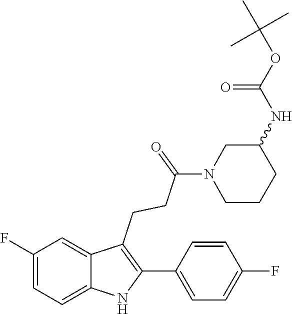 [SMILES: CC(C)(C)OC(=O)NC1CCCN(C(=O)CCc2c(-c3ccc(F)cc3)[nH]c3ccc(F)cc23)C1] | 66 |
| 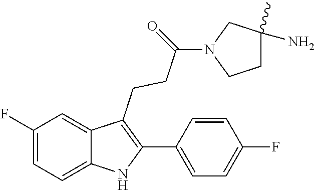 [SMILES: CC1(N)CCN(C(=O)CCc2c(-c3ccc(F)cc3)[nH]c3ccc(F)cc23)C1] | 67 |
| 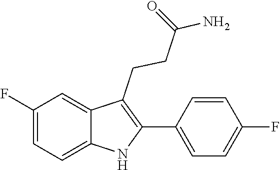 [SMILES: NC(=O)CCc1c(-c2ccc(F)cc2)[nH]c2ccc(F)cc12] | 68 |
| 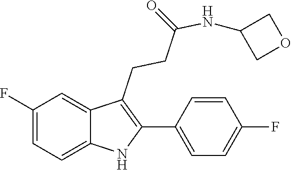 [SMILES: O=C(CCc1c(-c2ccc(F)cc2)[nH]c2ccc(F)cc12)NC1COC1] | 69 |
| 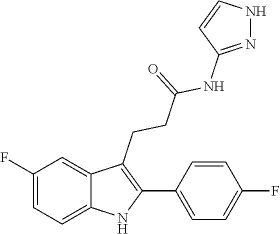 [SMILES: O=C(CCc1c(-c2ccc(F)cc2)[nH]c2ccc(F)cc12)Nc1cc[nH]n1] | 70 |
| 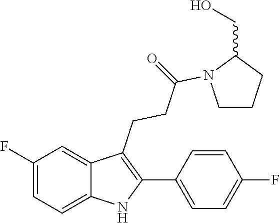 [SMILES: O=C(CCc1c(-c2ccc(F)cc2)[nH]c2ccc(F)cc12)N1CCCC1CO] | 71 |
| 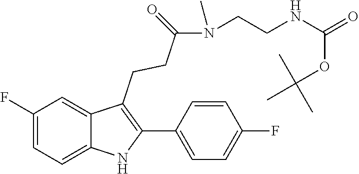 [SMILES: CN(CCNC(=O)OC(C)(C)C)C(=O)CCc1c(-c2ccc(F)cc2)[nH]c2ccc(F)cc12] | 72 |
| 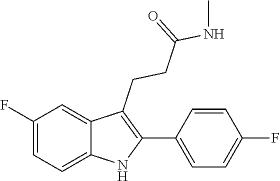 [SMILES: CNC(=O)CCc1c(-c2ccc(F)cc2)[nH]c2ccc(F)cc12] | 73 |
| 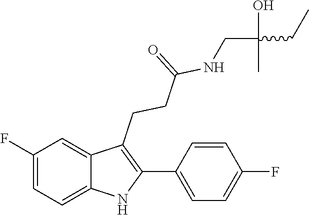 [SMILES: CCC(C)(O)CNC(=O)CCc1c(-c2ccc(F)cc2)[nH]c2ccc(F)cc12] | 74 |
| 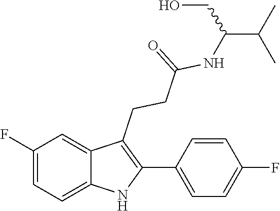 [SMILES: CC(C)C(CO)NC(=O)CCc1c(-c2ccc(F)cc2)[nH]c2ccc(F)cc12] | 75 |
| 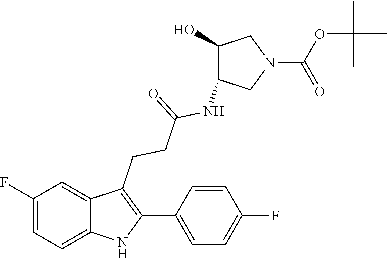 [SMILES: CC(C)(C)OC(=O)N1C[C@H](NC(=O)CCc2c(-c3ccc(F)cc3)[nH]c3ccc(F)cc23)[C@@H](O)C1] | 76 |
| 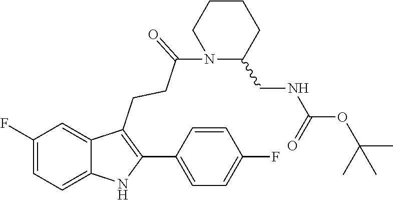 [SMILES: CC(C)(C)OC(=O)NCC1CCCCN1C(=O)CCc1c(-c2ccc(F)cc2)[nH]c2ccc(F)cc12] | 77 |
| 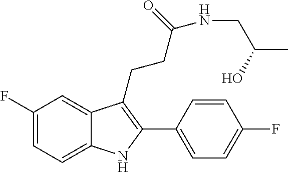 [SMILES: C[C@H](O)CNC(=O)CCc1c(-c2ccc(F)cc2)[nH]c2ccc(F)cc12] | 78 |
| 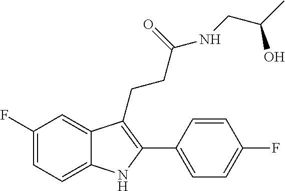 [SMILES: C[C@@H](O)CNC(=O)CCc1c(-c2ccc(F)cc2)[nH]c2ccc(F)cc12] | 79 |
| 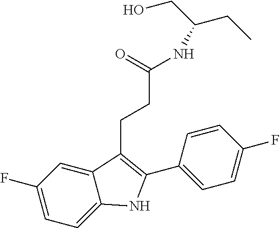 [SMILES: CC[C@@H](CO)NC(=O)CCc1c(-c2ccc(F)cc2)[nH]c2ccc(F)cc12] | 80 |
| 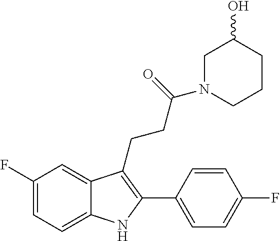 [SMILES: O=C(CCc1c(-c2ccc(F)cc2)[nH]c2ccc(F)cc12)N1CCCC(O)C1] | 81 |
| 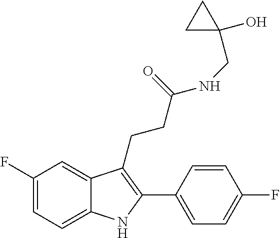 [SMILES: O=C(CCc1c(-c2ccc(F)cc2)[nH]c2ccc(F)cc12)NCC1(O)CC1] | 82 |
| 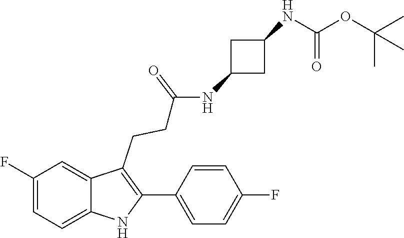 [SMILES: CC(C)(C)OC(=O)N[C@H]1C[C@@H](NC(=O)CCc2c(-c3ccc(F)cc3)[nH]c3ccc(F)cc23)C1] | 83 |
| 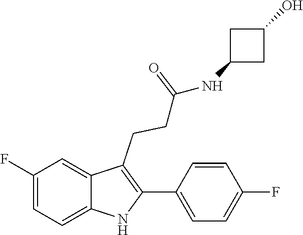 [SMILES: O=C(CCc1c(-c2ccc(F)cc2)[nH]c2ccc(F)cc12)N[C@H]1C[C@H](O)C1] | 84 |
| 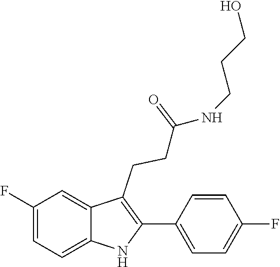 [SMILES: O=C(CCc1c(-c2ccc(F)cc2)[nH]c2ccc(F)cc12)NCCCO] | 85 |
| 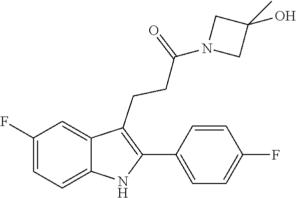 [SMILES: CC1(O)CN(C(=O)CCc2c(-c3ccc(F)cc3)[nH]c3ccc(F)cc23)C1] | 86 |
| 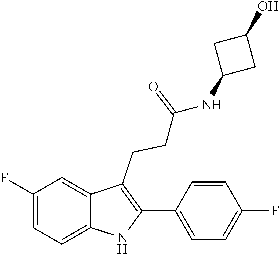 [SMILES: O=C(CCc1c(-c2ccc(F)cc2)[nH]c2ccc(F)cc12)N[C@H]1C[C@@H](O)C1] | 87 |
| 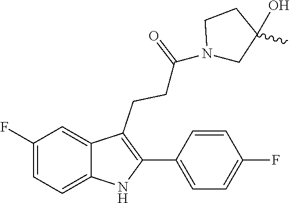 [SMILES: CC1(O)CCN(C(=O)CCc2c(-c3ccc(F)cc3)[nH]c3ccc(F)cc23)C1] | 88 |
| 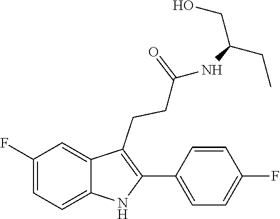 [SMILES: CC[C@H](CO)NC(=O)CCc1c(-c2ccc(F)cc2)[nH]c2ccc(F)cc12] | 89 |
| 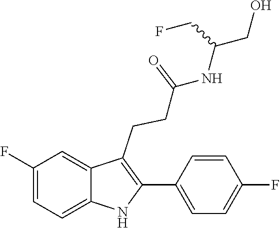 [SMILES: O=C(CCc1c(-c2ccc(F)cc2)[nH]c2ccc(F)cc12)NC(CO)CF] | 90 |
| 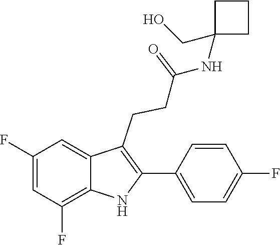 [SMILES: O=C(CCc1c(-c2ccc(F)cc2)[nH]c2c(F)cc(F)cc12)NC1(CO)CCC1] | 91 |
| 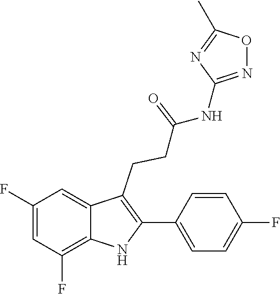 [SMILES: Cc1nc(NC(=O)CCc2c(-c3ccc(F)cc3)[nH]c3c(F)cc(F)cc23)no1] | 92 |
| 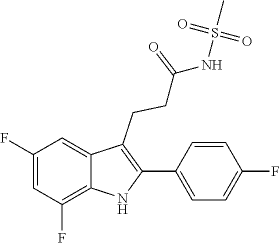 [SMILES: CS(=O)(=O)NC(=O)CCc1c(-c2ccc(F)cc2)[nH]c2c(F)cc(F)cc12] | 93 |
| 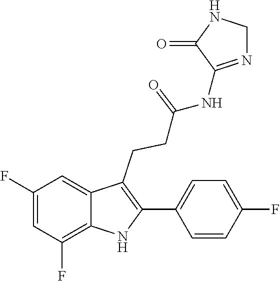 [SMILES: O=C(CCc1c(-c2ccc(F)cc2)[nH]c2c(F)cc(F)cc12)NC1=NCNC1=O] | 94 |
| 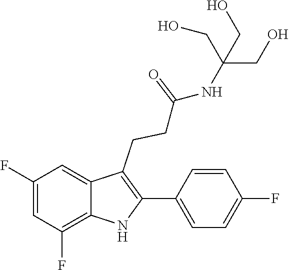 [SMILES: O=C(CCc1c(-c2ccc(F)cc2)[nH]c2c(F)cc(F)cc12)NC(CO)(CO)CO] | 95 |
| 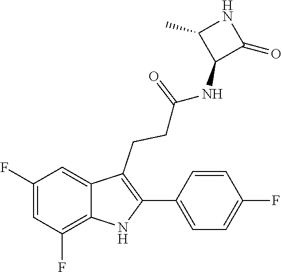 [SMILES: C[C@@H]1NC(=O)[C@H]1NC(=O)CCc1c(-c2ccc(F)cc2)[nH]c2c(F)cc(F)cc12] | 96 |
| 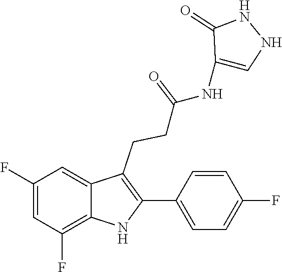 [SMILES: O=C(CCc1c(-c2ccc(F)cc2)[nH]c2c(F)cc(F)cc12)Nc1c[nH][nH]c1=O] | 97 |
| 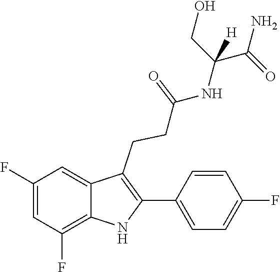 [SMILES: NC(=O)[C@@H](CO)NC(=O)CCc1c(-c2ccc(F)cc2)[nH]c2c(F)cc(F)cc12] | 98 |
| 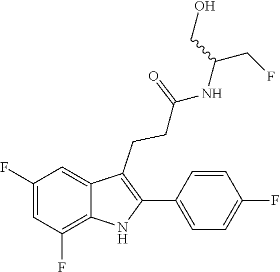 [SMILES: O=C(CCc1c(-c2ccc(F)cc2)[nH]c2c(F)cc(F)cc12)NC(CO)CF] | 99 |
| 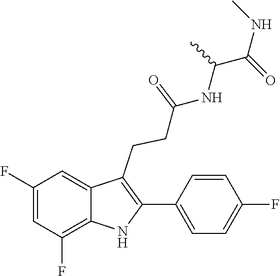 [SMILES: CNC(=O)C(C)NC(=O)CCc1c(-c2ccc(F)cc2)[nH]c2c(F)cc(F)cc12] | 100 |
| 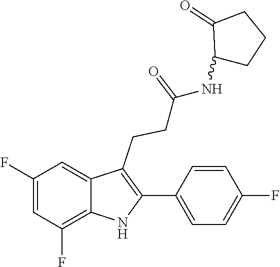 [SMILES: O=C(CCc1c(-c2ccc(F)cc2)[nH]c2c(F)cc(F)cc12)NC1CCCC1=O] | 101 |
| 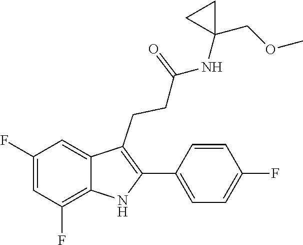 [SMILES: COCC1(NC(=O)CCc2c(-c3ccc(F)cc3)[nH]c3c(F)cc(F)cc23)CC1] | 102 |
| 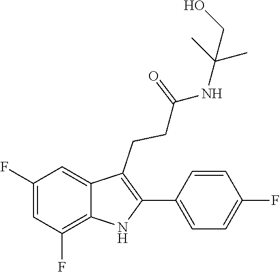 [SMILES: CC(C)(CO)NC(=O)CCc1c(-c2ccc(F)cc2)[nH]c2c(F)cc(F)cc12] | 103 |
| 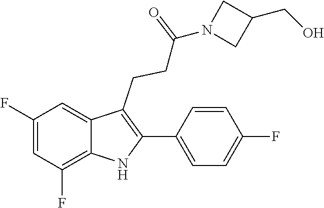 [SMILES: O=C(CCc1c(-c2ccc(F)cc2)[nH]c2c(F)cc(F)cc12)N1CC(CO)C1] | 104 |
| 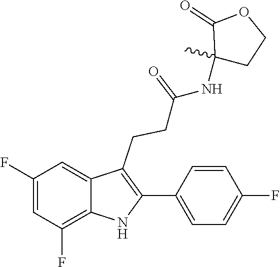 [SMILES: CC1(NC(=O)CCc2c(-c3ccc(F)cc3)[nH]c3c(F)cc(F)cc23)CCOC1=O] | 105 |
| 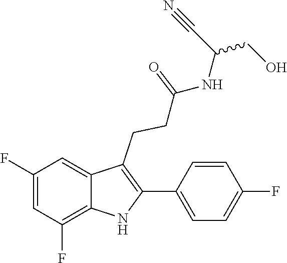 [SMILES: N#CC(CO)NC(=O)CCc1c(-c2ccc(F)cc2)[nH]c2c(F)cc(F)cc12] | 106 |
| 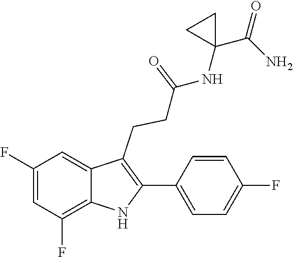 [SMILES: NC(=O)C1(NC(=O)CCc2c(-c3ccc(F)cc3)[nH]c3c(F)cc(F)cc23)CC1] | 107 |
| 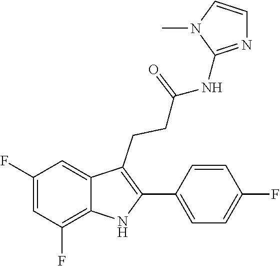 [SMILES: Cn1ccnc1NC(=O)CCc1c(-c2ccc(F)cc2)[nH]c2c(F)cc(F)cc12] | 108 |
| 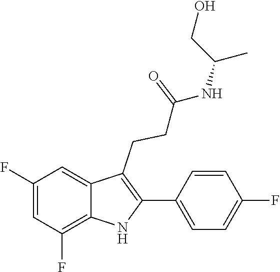 [SMILES: C[C@@H](CO)NC(=O)CCc1c(-c2ccc(F)cc2)[nH]c2c(F)cc(F)cc12] | 109 |
| 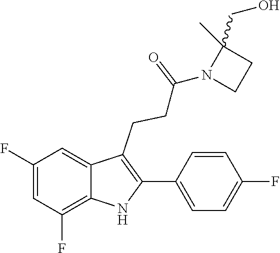 [SMILES: CC1(CO)CCN1C(=O)CCc1c(-c2ccc(F)cc2)[nH]c2c(F)cc(F)cc12] | 110 |
| 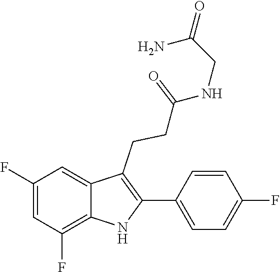 [SMILES: NC(=O)CNC(=O)CCc1c(-c2ccc(F)cc2)[nH]c2c(F)cc(F)cc12] | 111 |
| 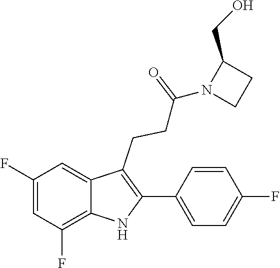 [SMILES: O=C(CCc1c(-c2ccc(F)cc2)[nH]c2c(F)cc(F)cc12)N1CC[C@@H]1CO] | 112 |
| 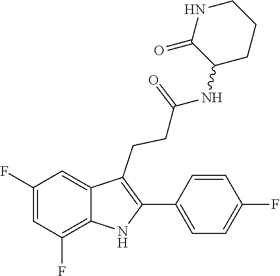 [SMILES: O=C(CCc1c(-c2ccc(F)cc2)[nH]c2c(F)cc(F)cc12)NC1CCCNC1=O] | 113 |
| 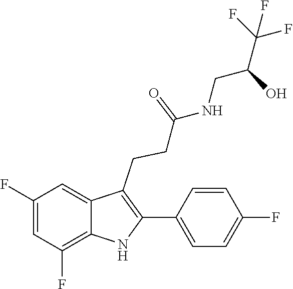 [SMILES: O=C(CCc1c(-c2ccc(F)cc2)[nH]c2c(F)cc(F)cc12)NC[C@H](O)C(F)(F)F] | 114 |
| 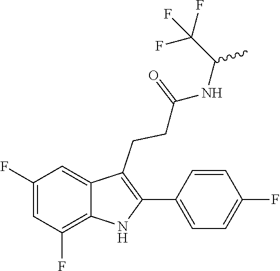 [SMILES: CC(NC(=O)CCc1c(-c2ccc(F)cc2)[nH]c2c(F)cc(F)cc12)C(F)(F)F] | 115 |
| 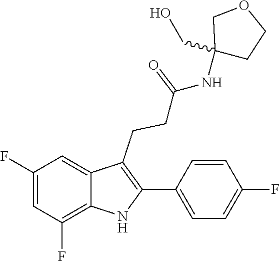 [SMILES: O=C(CCc1c(-c2ccc(F)cc2)[nH]c2c(F)cc(F)cc12)NC1(CO)CCOC1] | 116 |
| 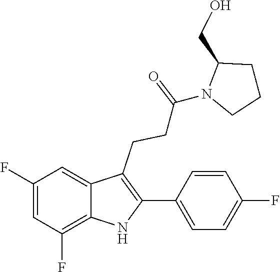 [SMILES: O=C(CCc1c(-c2ccc(F)cc2)[nH]c2c(F)cc(F)cc12)N1CCC[C@@H]1CO] | 117 |
| 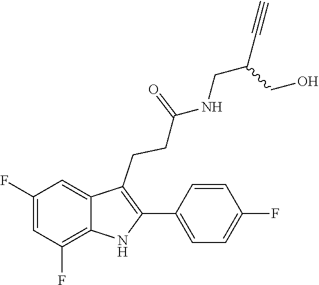 [SMILES: C#CC(CO)CNC(=O)CCc1c(-c2ccc(F)cc2)[nH]c2c(F)cc(F)cc12] | 118 |
| 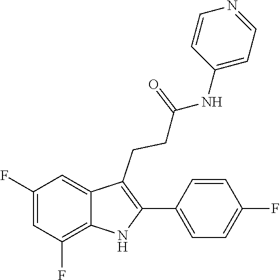 [SMILES: O=C(CCc1c(-c2ccc(F)cc2)[nH]c2c(F)cc(F)cc12)Nc1ccncc1] | 119 |
| 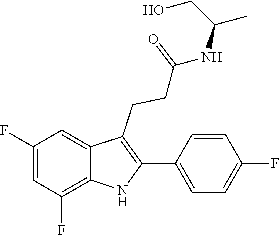 [SMILES: C[C@H](CO)NC(=O)CCc1c(-c2ccc(F)cc2)[nH]c2c(F)cc(F)cc12] | 120 |
| 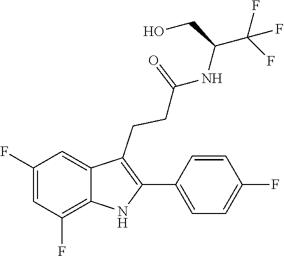 [SMILES: O=C(CCc1c(-c2ccc(F)cc2)[nH]c2c(F)cc(F)cc12)N[C@@H](CO)C(F)(F)F] | 121 |
| 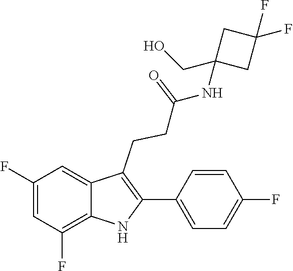 [SMILES: O=C(CCc1c(-c2ccc(F)cc2)[nH]c2c(F)cc(F)cc12)NC1(CO)CC(F)(F)C1] | 122 |
| 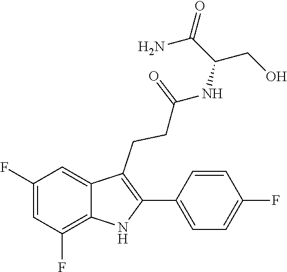 [SMILES: NC(=O)[C@H](CO)NC(=O)CCc1c(-c2ccc(F)cc2)[nH]c2c(F)cc(F)cc12] | 123 |
| 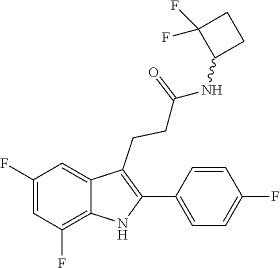 [SMILES: O=C(CCc1c(-c2ccc(F)cc2)[nH]c2c(F)cc(F)cc12)NC1CCC1(F)F] | 124 |
| 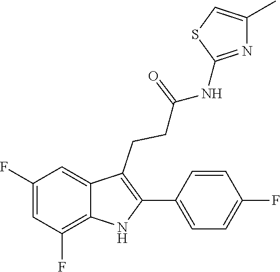 [SMILES: Cc1csc(NC(=O)CCc2c(-c3ccc(F)cc3)[nH]c3c(F)cc(F)cc23)n1] | 125 |
| 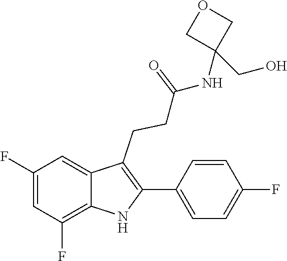 [SMILES: O=C(CCc1c(-c2ccc(F)cc2)[nH]c2c(F)cc(F)cc12)NC1(CO)COC1] | 126 |
| 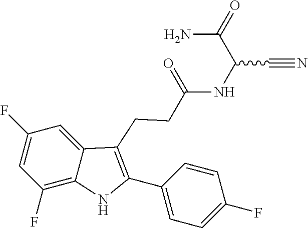 [SMILES: N#CC(NC(=O)CCc1c(-c2ccc(F)cc2)[nH]c2c(F)cc(F)cc12)C(N)=O] | 127 |
| 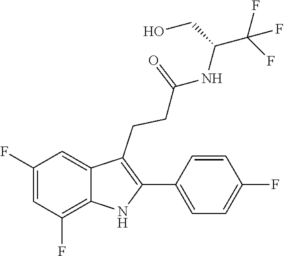 [SMILES: O=C(CCc1c(-c2ccc(F)cc2)[nH]c2c(F)cc(F)cc12)N[C@H](CO)C(F)(F)F] | 128 |
| 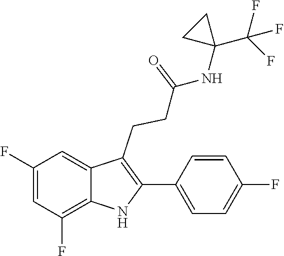 [SMILES: O=C(CCc1c(-c2ccc(F)cc2)[nH]c2c(F)cc(F)cc12)NC1(C(F)(F)F)CC1] | 129 |
| 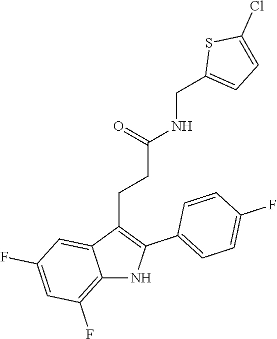 [SMILES: O=C(CCc1c(-c2ccc(F)cc2)[nH]c2c(F)cc(F)cc12)NCc1ccc(Cl)s1] | 130 |
| 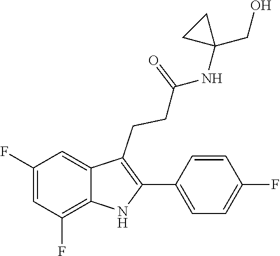 [SMILES: O=C(CCc1c(-c2ccc(F)cc2)[nH]c2c(F)cc(F)cc12)NC1(CO)CC1] | 131 |
| 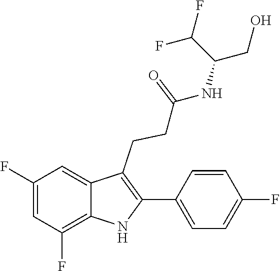 [SMILES: O=C(CCc1c(-c2ccc(F)cc2)[nH]c2c(F)cc(F)cc12)N[C@@H](CO)C(F)F] | 132 |
| 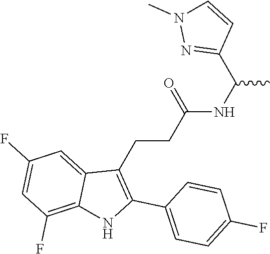 [SMILES: CC(NC(=O)CCc1c(-c2ccc(F)cc2)[nH]c2c(F)cc(F)cc12)c1ccn(C)n1] | 133 |
| 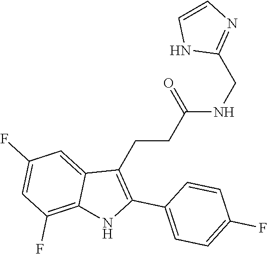 [SMILES: O=C(CCc1c(-c2ccc(F)cc2)[nH]c2c(F)cc(F)cc12)NCc1ncc[nH]1] | 134 |
| 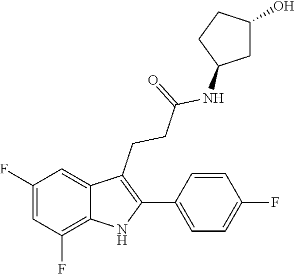 [SMILES: O=C(CCc1c(-c2ccc(F)cc2)[nH]c2c(F)cc(F)cc12)N[C@H]1CC[C@H](O)C1] | 135 |
| 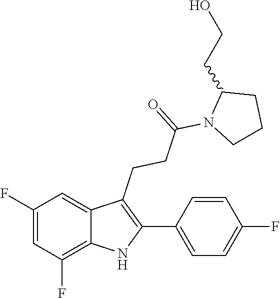 [SMILES: O=C(CCc1c(-c2ccc(F)cc2)[nH]c2c(F)cc(F)cc12)N1CCCC1CCO] | 136 |
| 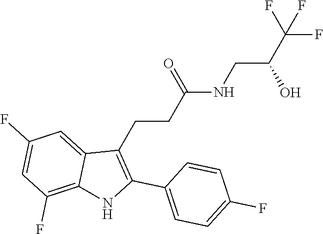 [SMILES: O=C(CCc1c(-c2ccc(F)cc2)[nH]c2c(F)cc(F)cc12)NC[C@@H](O)C(F)(F)F] | 137 |
| 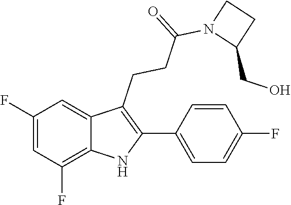 [SMILES: O=C(CCc1c(-c2ccc(F)cc2)[nH]c2c(F)cc(F)cc12)N1CC[C@H]1CO] | 138 |
| 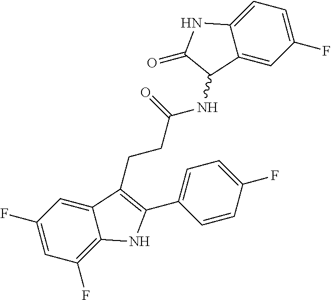 [SMILES: O=C(CCc1c(-c2ccc(F)cc2)[nH]c2c(F)cc(F)cc12)NC1C(=O)Nc2ccc(F)cc21] | 139 |
| 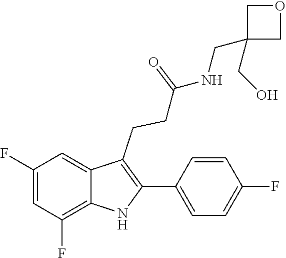 [SMILES: O=C(CCc1c(-c2ccc(F)cc2)[nH]c2c(F)cc(F)cc12)NCC1(CO)COC1] | 140 |
| 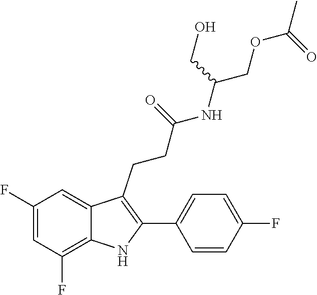 [SMILES: CC(=O)OCC(CO)NC(=O)CCc1c(-c2ccc(F)cc2)[nH]c2c(F)cc(F)cc12] | 141 |
| 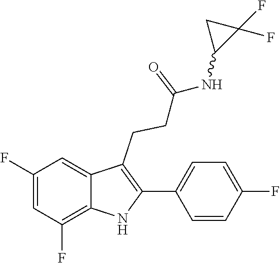 [SMILES: O=C(CCc1c(-c2ccc(F)cc2)[nH]c2c(F)cc(F)cc12)NC1CC1(F)F] | 142 |
| 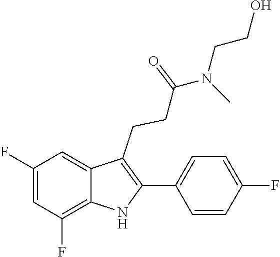 [SMILES: CN(CCO)C(=O)CCc1c(-c2ccc(F)cc2)[nH]c2c(F)cc(F)cc12] | 143 |
| 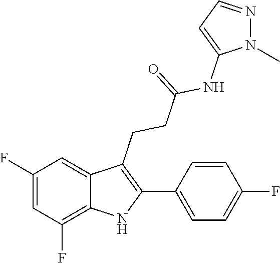 [SMILES: Cn1nccc1NC(=O)CCc1c(-c2ccc(F)cc2)[nH]c2c(F)cc(F)cc12] | 144 |
| 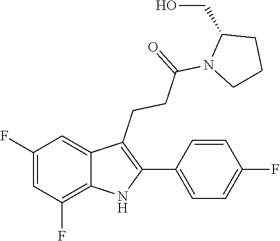 [SMILES: O=C(CCc1c(-c2ccc(F)cc2)[nH]c2c(F)cc(F)cc12)N1CCC[C@H]1CO] | 145 |
| 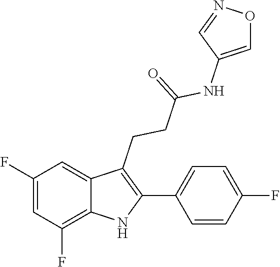 [SMILES: O=C(CCc1c(-c2ccc(F)cc2)[nH]c2c(F)cc(F)cc12)Nc1cnoc1] | 146 |
| 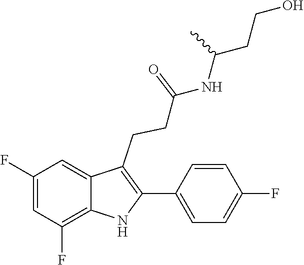 [SMILES: CC(CCO)NC(=O)CCc1c(-c2ccc(F)cc2)[nH]c2c(F)cc(F)cc12] | 147 |
| 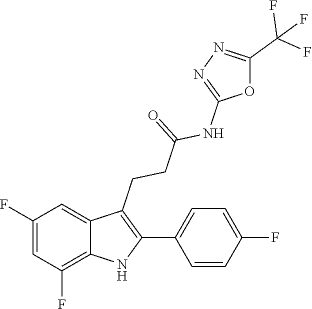 [SMILES: O=C(CCc1c(-c2ccc(F)cc2)[nH]c2c(F)cc(F)cc12)Nc1nnc(C(F)(F)F)o1] | 148 |
| 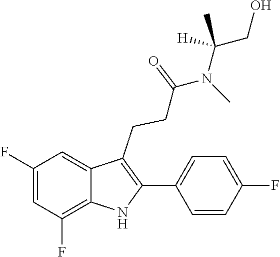 [SMILES: C[C@@H](CO)N(C)C(=O)CCc1c(-c2ccc(F)cc2)[nH]c2c(F)cc(F)cc12] | 149 |
| 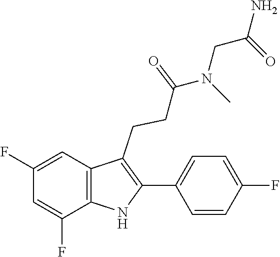 [SMILES: CN(CC(N)=O)C(=O)CCc1c(-c2ccc(F)cc2)[nH]c2c(F)cc(F)cc12] | 150 |
| 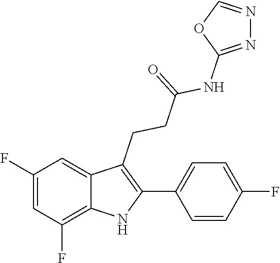 [SMILES: O=C(CCc1c(-c2ccc(F)cc2)[nH]c2c(F)cc(F)cc12)Nc1nnco1] | 151 |
| 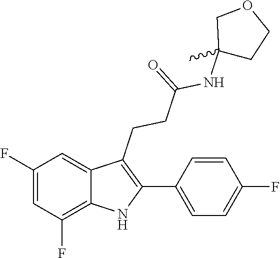 [SMILES: CC1(NC(=O)CCc2c(-c3ccc(F)cc3)[nH]c3c(F)cc(F)cc23)CCOC1] | 152 |
| 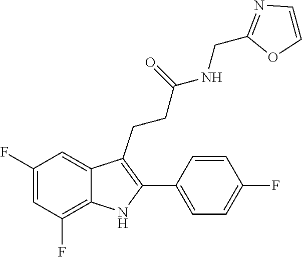 [SMILES: O=C(CCc1c(-c2ccc(F)cc2)[nH]c2c(F)cc(F)cc12)NCc1ncco1] | 153 |
| 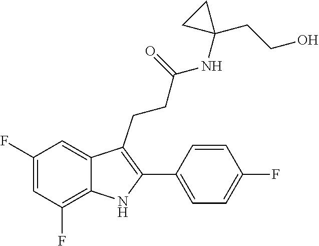 [SMILES: O=C(CCc1c(-c2ccc(F)cc2)[nH]c2c(F)cc(F)cc12)NC1(CCO)CC1] | 154 |
| 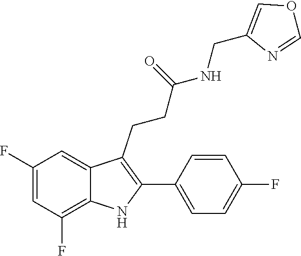 [SMILES: O=C(CCc1c(-c2ccc(F)cc2)[nH]c2c(F)cc(F)cc12)NCc1cocn1] | 155 |
| 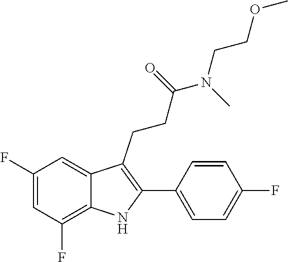 [SMILES: COCCN(C)C(=O)CCc1c(-c2ccc(F)cc2)[nH]c2c(F)cc(F)cc12] | 156 |
| 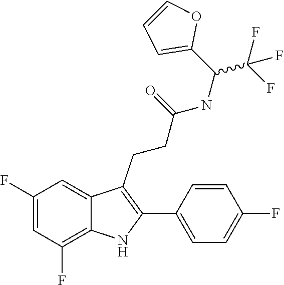 [SMILES: O=C(CCc1c(-c2ccc(F)cc2)[nH]c2c(F)cc(F)cc12)NC(c1ccco1)C(F)(F)F] | 157 |
| 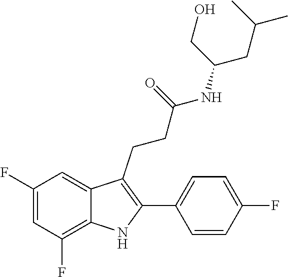 [SMILES: CC(C)C[C@@H](CO)NC(=O)CCc1c(-c2ccc(F)cc2)[nH]c2c(F)cc(F)cc12] | 158 |
| 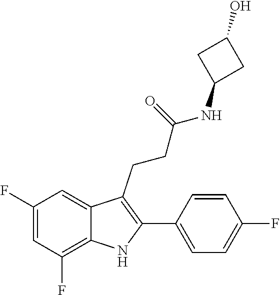 [SMILES: O=C(CCc1c(-c2ccc(F)cc2)[nH]c2c(F)cc(F)cc12)N[C@H]1C[C@H](O)C1] | 159 |
| 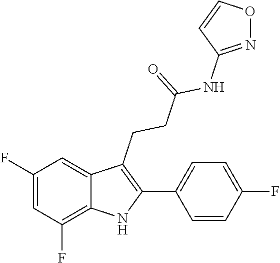 [SMILES: O=C(CCc1c(-c2ccc(F)cc2)[nH]c2c(F)cc(F)cc12)Nc1ccon1] | 160 |
| 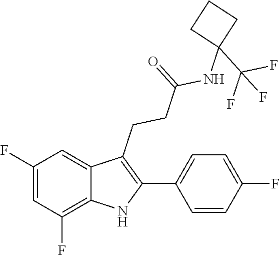 [SMILES: O=C(CCc1c(-c2ccc(F)cc2)[nH]c2c(F)cc(F)cc12)NC1(C(F)(F)F)CCC1] | 161 |
| 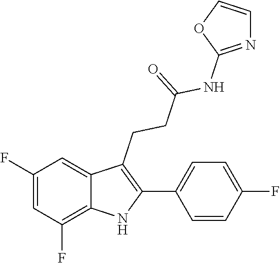 [SMILES: O=C(CCc1c(-c2ccc(F)cc2)[nH]c2c(F)cc(F)cc12)Nc1ncco1] | 162 |
| 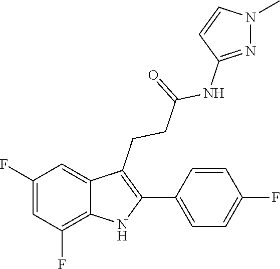 [SMILES: Cn1ccc(NC(=O)CCc2c(-c3ccc(F)cc3)[nH]c3c(F)cc(F)cc23)n1] | 163 |
| 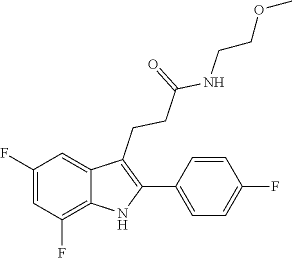 [SMILES: COCCNC(=O)CCc1c(-c2ccc(F)cc2)[nH]c2c(F)cc(F)cc12] | 164 |
| 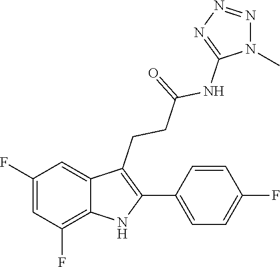 [SMILES: Cn1nnnc1NC(=O)CCc1c(-c2ccc(F)cc2)[nH]c2c(F)cc(F)cc12] | 165 |
| 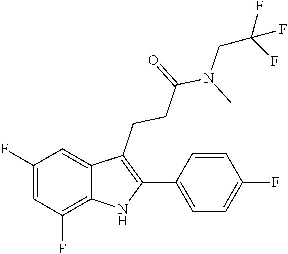 [SMILES: CN(CC(F)(F)F)C(=O)CCc1c(-c2ccc(F)cc2)[nH]c2c(F)cc(F)cc12] | 166 |
| 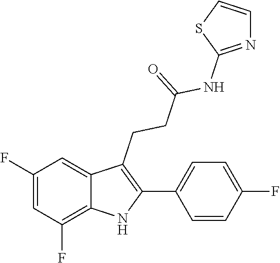 [SMILES: O=C(CCc1c(-c2ccc(F)cc2)[nH]c2c(F)cc(F)cc12)Nc1nccs1] | 167 |
| 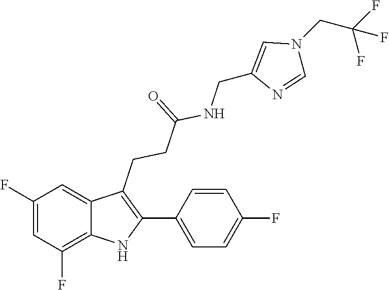 [SMILES: O=C(CCc1c(-c2ccc(F)cc2)[nH]c2c(F)cc(F)cc12)NCc1cn(CC(F)(F)F)cn1] | 168 |
| 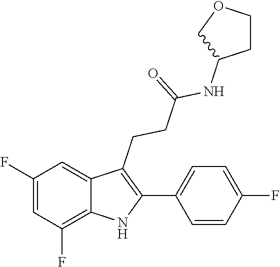 [SMILES: O=C(CCc1c(-c2ccc(F)cc2)[nH]c2c(F)cc(F)cc12)NC1CCOC1] | 169 |
| 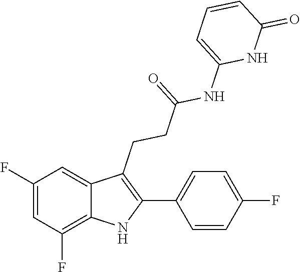 [SMILES: O=C(CCc1c(-c2ccc(F)cc2)[nH]c2c(F)cc(F)cc12)Nc1cccc(=O)[nH]1] | 170 |
| 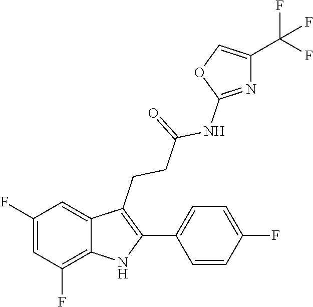 [SMILES: O=C(CCc1c(-c2ccc(F)cc2)[nH]c2c(F)cc(F)cc12)Nc1nc(C(F)(F)F)co1] | 171 |
| 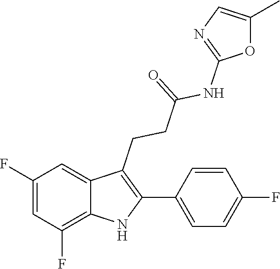 [SMILES: Cc1cnc(NC(=O)CCc2c(-c3ccc(F)cc3)[nH]c3c(F)cc(F)cc23)o1] | 172 |
| 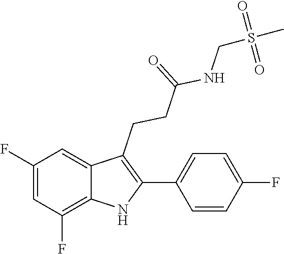 [SMILES: CS(=O)(=O)CNC(=O)CCc1c(-c2ccc(F)cc2)[nH]c2c(F)cc(F)cc12] | 173 |
| 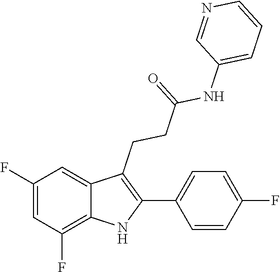 [SMILES: O=C(CCc1c(-c2ccc(F)cc2)[nH]c2c(F)cc(F)cc12)Nc1cccnc1] | 174 |
| 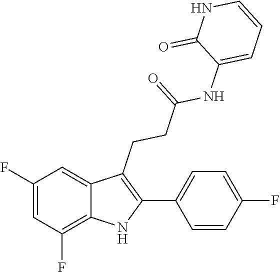 [SMILES: O=C(CCc1c(-c2ccc(F)cc2)[nH]c2c(F)cc(F)cc12)Nc1ccc[nH]c1=O] | 175 |
| 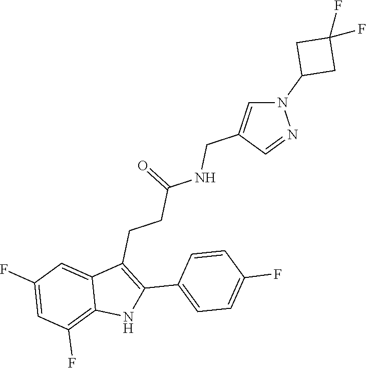 [SMILES: O=C(CCc1c(-c2ccc(F)cc2)[nH]c2c(F)cc(F)cc12)NCc1cnn(C2CC(F)(F)C2)c1] | 176 |
| 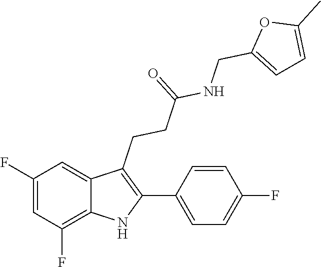 [SMILES: Cc1ccc(CNC(=O)CCc2c(-c3ccc(F)cc3)[nH]c3c(F)cc(F)cc23)o1] | 177 |
| 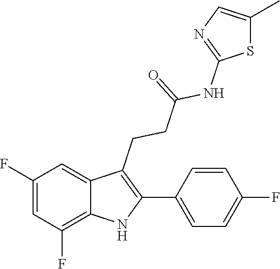 [SMILES: Cc1cnc(NC(=O)CCc2c(-c3ccc(F)cc3)[nH]c3c(F)cc(F)cc23)s1] | 178 |
| 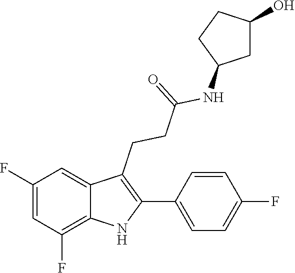 [SMILES: O=C(CCc1c(-c2ccc(F)cc2)[nH]c2c(F)cc(F)cc12)N[C@H]1CC[C@@H](O)C1] | 179 |
| 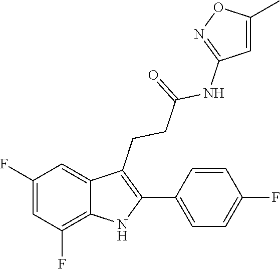 [SMILES: Cc1cc(NC(=O)CCc2c(-c3ccc(F)cc3)[nH]c3c(F)cc(F)cc23)no1] | 180 |
| 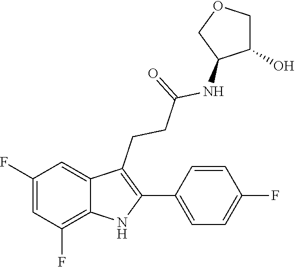 [SMILES: O=C(CCc1c(-c2ccc(F)cc2)[nH]c2c(F)cc(F)cc12)N[C@H]1COC[C@@H]1O] | 181 |
| 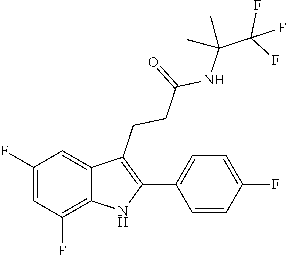 [SMILES: CC(C)(NC(=O)CCc1c(-c2ccc(F)cc2)[nH]c2c(F)cc(F)cc12)C(F)(F)F] | 182 |
| 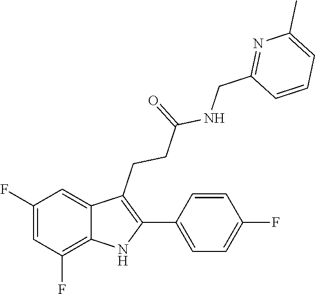 [SMILES: Cc1cccc(CNC(=O)CCc2c(-c3ccc(F)cc3)[nH]c3c(F)cc(F)cc23)n1] | 183 |
| 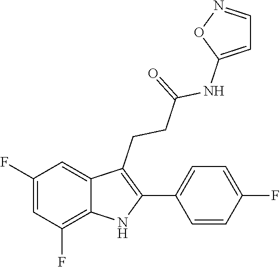 [SMILES: O=C(CCc1c(-c2ccc(F)cc2)[nH]c2c(F)cc(F)cc12)Nc1ccno1] | 184 |
| 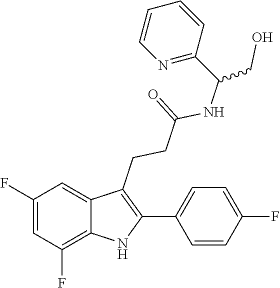 [SMILES: O=C(CCc1c(-c2ccc(F)cc2)[nH]c2c(F)cc(F)cc12)NC(CO)c1ccccn1] | 185 |
| 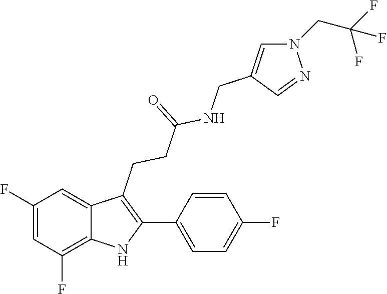 [SMILES: O=C(CCc1c(-c2ccc(F)cc2)[nH]c2c(F)cc(F)cc12)NCc1cnn(CC(F)(F)F)c1] | 186 |
| 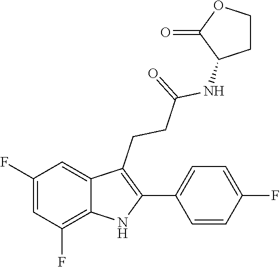 [SMILES: O=C(CCc1c(-c2ccc(F)cc2)[nH]c2c(F)cc(F)cc12)N[C@H]1CCOC1=O] | 187 |
| 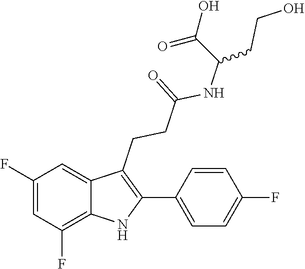 [SMILES: O=C(CCc1c(-c2ccc(F)cc2)[nH]c2c(F)cc(F)cc12)NC(CCO)C(=O)O] | 188 |
| 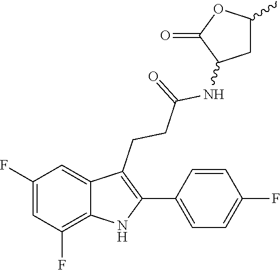 [SMILES: CC1CC(NC(=O)CCc2c(-c3ccc(F)cc3)[nH]c3c(F)cc(F)cc23)C(=O)O1] | 189 |
| 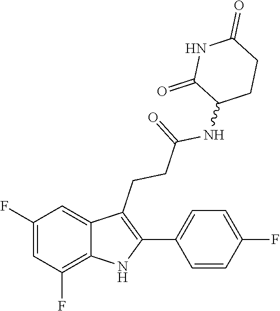 [SMILES: O=C1CCC(NC(=O)CCc2c(-c3ccc(F)cc3)[nH]c3c(F)cc(F)cc23)C(=O)N1] | 190 |
| 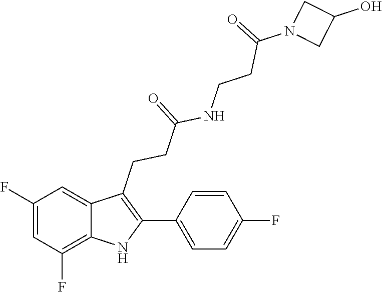 [SMILES: O=C(CCc1c(-c2ccc(F)cc2)[nH]c2c(F)cc(F)cc12)NCCC(=O)N1CC(O)C1] | 191 |
| 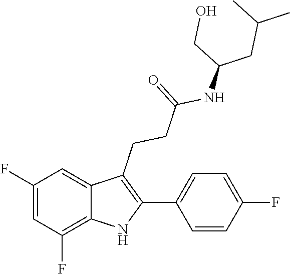 [SMILES: CC(C)C[C@H](CO)NC(=O)CCc1c(-c2ccc(F)cc2)[nH]c2c(F)cc(F)cc12] | 192 |
| 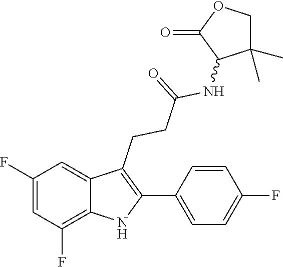 [SMILES: CC1(C)COC(=O)C1NC(=O)CCc1c(-c2ccc(F)cc2)[nH]c2c(F)cc(F)cc12] | 193 |
| 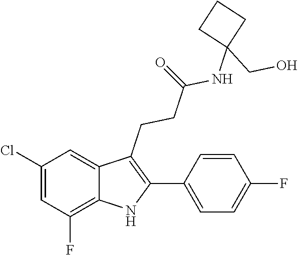 [SMILES: O=C(CCc1c(-c2ccc(F)cc2)[nH]c2c(F)cc(Cl)cc12)NC1(CO)CCC1] | 194 |
| 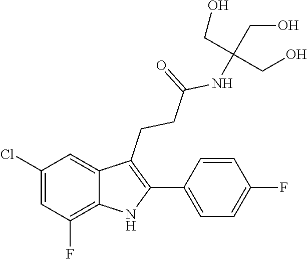 [SMILES: O=C(CCc1c(-c2ccc(F)cc2)[nH]c2c(F)cc(Cl)cc12)NC(CO)(CO)CO] | 195 |
| 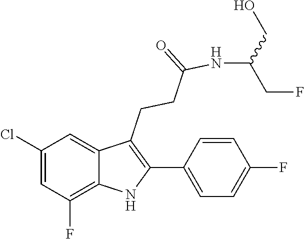 [SMILES: O=C(CCc1c(-c2ccc(F)cc2)[nH]c2c(F)cc(Cl)cc12)NC(CO)CF] | 196 |
| 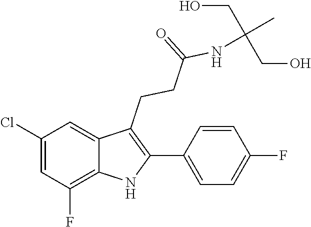 [SMILES: CC(CO)(CO)NC(=O)CCc1c(-c2ccc(F)cc2)[nH]c2c(F)cc(Cl)cc12] | 197 |
| 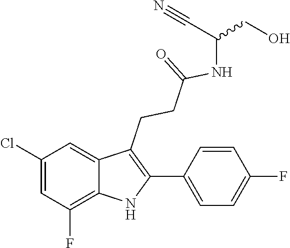 [SMILES: N#CC(CO)NC(=O)CCc1c(-c2ccc(F)cc2)[nH]c2c(F)cc(Cl)cc12] | 198 |
| 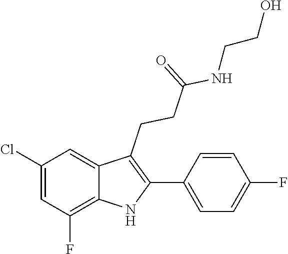 [SMILES: O=C(CCc1c(-c2ccc(F)cc2)[nH]c2c(F)cc(Cl)cc12)NCCO] | 199 |
| 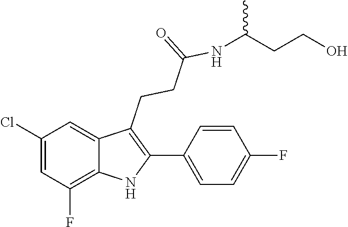 [SMILES: CC(CCO)NC(=O)CCc1c(-c2ccc(F)cc2)[nH]c2c(F)cc(Cl)cc12] | 200 |
| 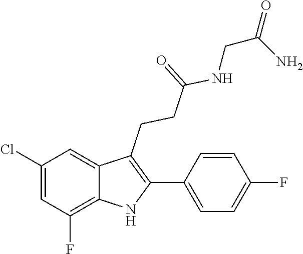 [SMILES: NC(=O)CNC(=O)CCc1c(-c2ccc(F)cc2)[nH]c2c(F)cc(Cl)cc12] | 201 |
| 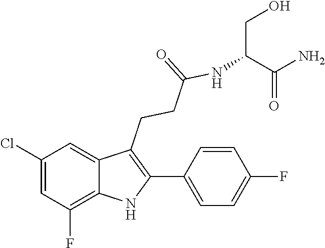 [SMILES: NC(=O)[C@@H](CO)NC(=O)CCc1c(-c2ccc(F)cc2)[nH]c2c(F)cc(Cl)cc12] | 202 |
| 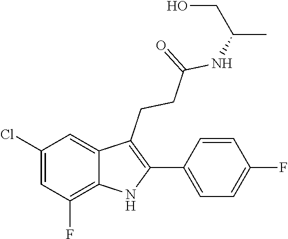 [SMILES: C[C@@H](CO)NC(=O)CCc1c(-c2ccc(F)cc2)[nH]c2c(F)cc(Cl)cc12] | 203 |
| 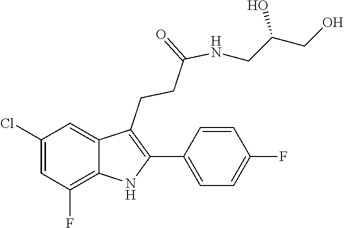 [SMILES: O=C(CCc1c(-c2ccc(F)cc2)[nH]c2c(F)cc(Cl)cc12)NC[C@H](O)CO] | 204 |
| 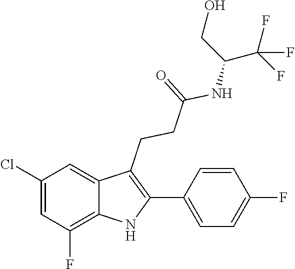 [SMILES: O=C(CCc1c(-c2ccc(F)cc2)[nH]c2c(F)cc(Cl)cc12)N[C@H](CO)C(F)(F)F] | 205 |
| 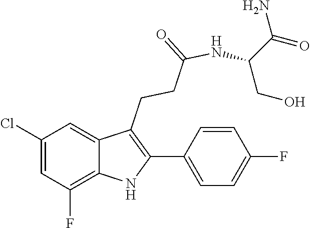 [SMILES: NC(=O)[C@H](CO)NC(=O)CCc1c(-c2ccc(F)cc2)[nH]c2c(F)cc(Cl)cc12] | 206 |
| 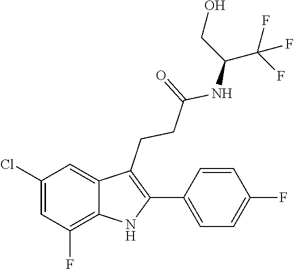 [SMILES: O=C(CCc1c(-c2ccc(F)cc2)[nH]c2c(F)cc(Cl)cc12)N[C@@H](CO)C(F)(F)F] | 207 |
| 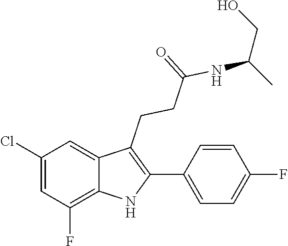 [SMILES: C[C@H](CO)NC(=O)CCc1c(-c2ccc(F)cc2)[nH]c2c(F)cc(Cl)cc12] | 208 |
| 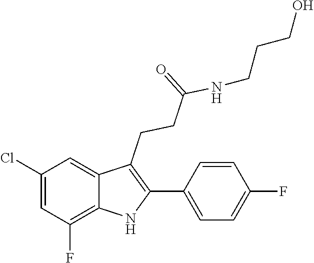 [SMILES: O=C(CCc1c(-c2ccc(F)cc2)[nH]c2c(F)cc(Cl)cc12)NCCCO] | 209 |
| 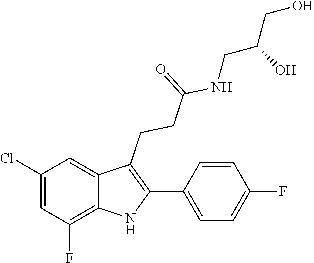 [SMILES: O=C(CCc1c(-c2ccc(F)cc2)[nH]c2c(F)cc(Cl)cc12)NC[C@@H](O)CO] | 210 |
| 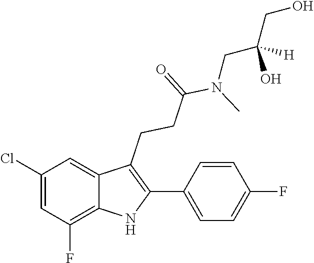 [SMILES: CN(C[C@H](O)CO)C(=O)CCc1c(-c2ccc(F)cc2)[nH]c2c(F)cc(Cl)cc12] | 211 |
| 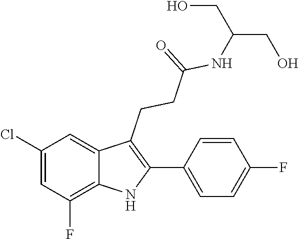 [SMILES: O=C(CCc1c(-c2ccc(F)cc2)[nH]c2c(F)cc(Cl)cc12)NC(CO)CO] | 212 |
| 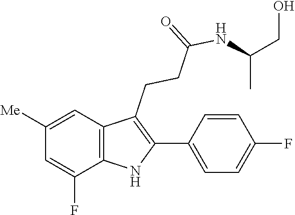 [SMILES: Cc1cc(F)c2[nH]c(-c3ccc(F)cc3)c(CCC(=O)N[C@H](C)CO)c2c1] | 213 |
| 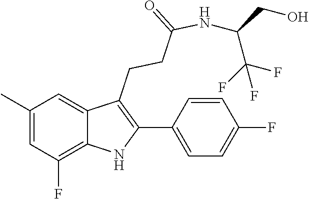 [SMILES: Cc1cc(F)c2[nH]c(-c3ccc(F)cc3)c(CCC(=O)N[C@@H](CO)C(F)(F)F)c2c1] | 214 |
| 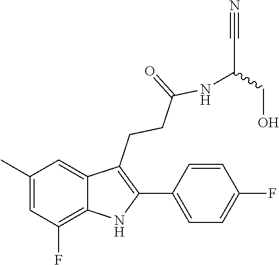 [SMILES: Cc1cc(F)c2[nH]c(-c3ccc(F)cc3)c(CCC(=O)NC(C#N)CO)c2c1] | 215 |
| 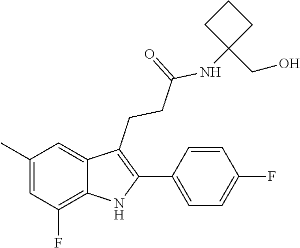 [SMILES: Cc1cc(F)c2[nH]c(-c3ccc(F)cc3)c(CCC(=O)NC3(CO)CCC3)c2c1] | 216 |
| 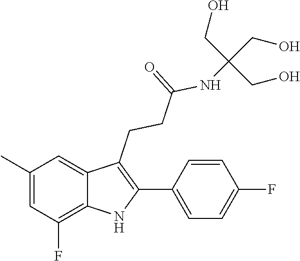 [SMILES: Cc1cc(F)c2[nH]c(-c3ccc(F)cc3)c(CCC(=O)NC(CO)(CO)CO)c2c1] | 217 |
| 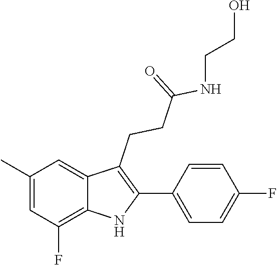 [SMILES: Cc1cc(F)c2[nH]c(-c3ccc(F)cc3)c(CCC(=O)NCCO)c2c1] | 218 |
| 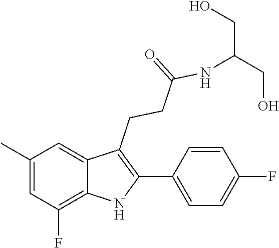 [SMILES: Cc1cc(F)c2[nH]c(-c3ccc(F)cc3)c(CCC(=O)NC(CO)CO)c2c1] | 219 |
| 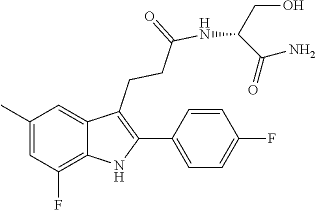 [SMILES: Cc1cc(F)c2[nH]c(-c3ccc(F)cc3)c(CCC(=O)N[C@H](CO)C(N)=O)c2c1] | 220 |
| 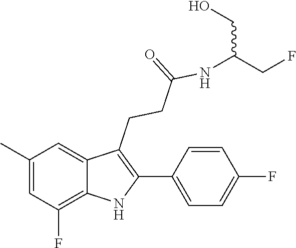 [SMILES: Cc1cc(F)c2[nH]c(-c3ccc(F)cc3)c(CCC(=O)NC(CO)CF)c2c1] | 221 |
| 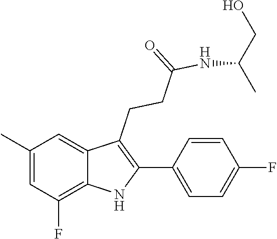 [SMILES: Cc1cc(F)c2[nH]c(-c3ccc(F)cc3)c(CCC(=O)N[C@@H](C)CO)c2c1] | 222 |
| 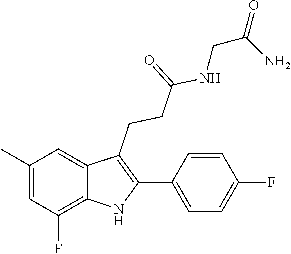 [SMILES: Cc1cc(F)c2[nH]c(-c3ccc(F)cc3)c(CCC(=O)NCC(N)=O)c2c1] | 223 |
| 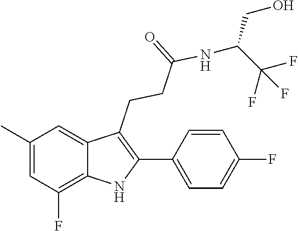 [SMILES: Cc1cc(F)c2[nH]c(-c3ccc(F)cc3)c(CCC(=O)N[C@H](CO)C(F)(F)F)c2c1] | 224 |
| 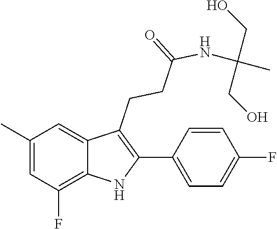 [SMILES: Cc1cc(F)c2[nH]c(-c3ccc(F)cc3)c(CCC(=O)NC(C)(CO)CO)c2c1] | 225 |
| 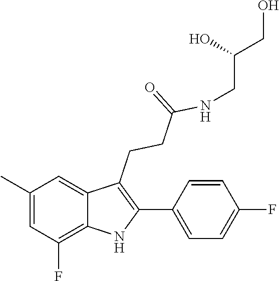 [SMILES: Cc1cc(F)c2[nH]c(-c3ccc(F)cc3)c(CCC(=O)NC[C@H](O)CO)c2c1] | 226 |
| 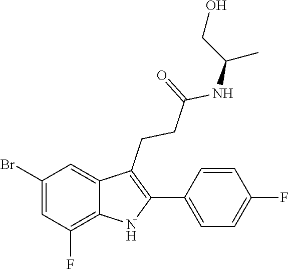 [SMILES: C[C@H](CO)NC(=O)CCc1c(-c2ccc(F)cc2)[nH]c2c(F)cc(Br)cc12] | 227 |
| 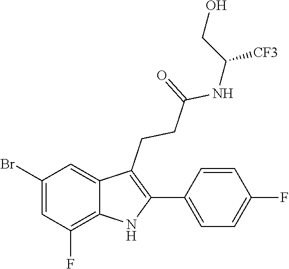 [SMILES: O=C(CCc1c(-c2ccc(F)cc2)[nH]c2c(F)cc(Br)cc12)N[C@H](CO)C(F)(F)F] | 228 |
| 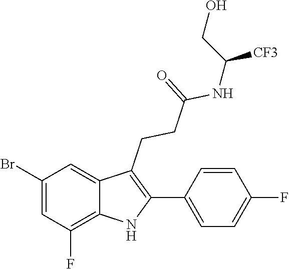 [SMILES: O=C(CCc1c(-c2ccc(F)cc2)[nH]c2c(F)cc(Br)cc12)N[C@@H](CO)C(F)(F)F] | 229 |
| 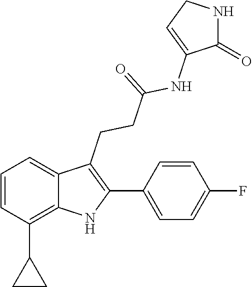 [SMILES: O=C(CCc1c(-c2ccc(F)cc2)[nH]c2c(C3CC3)cccc12)NC1=CCNC1=O] | 230 |
| 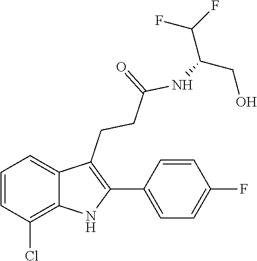 [SMILES: O=C(CCc1c(-c2ccc(F)cc2)[nH]c2c(Cl)cccc12)N[C@@H](CO)C(F)F] | 231 |
| 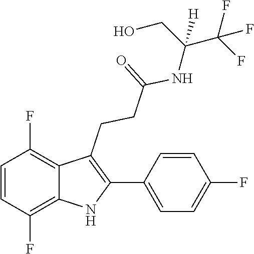 [SMILES: O=C(CCc1c(-c2ccc(F)cc2)[nH]c2c(F)ccc(F)c12)N[C@@H](CO)C(F)(F)F] | 232 |
| 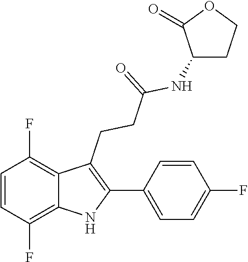 [SMILES: O=C(CCc1c(-c2ccc(F)cc2)[nH]c2c(F)ccc(F)c12)N[C@H]1CCOC1=O] | 233 |
| 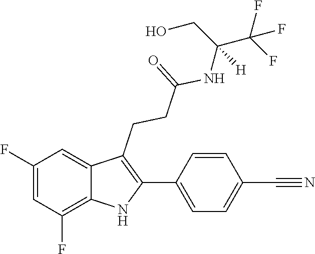 [SMILES: N#Cc1ccc(-c2[nH]c3c(F)cc(F)cc3c2CCC(=O)N[C@@H](CO)C(F)(F)F)cc1] | 234 |
| 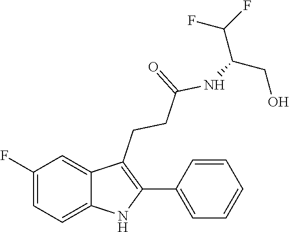 [SMILES: O=C(CCc1c(-c2ccccc2)[nH]c2ccc(F)cc12)N[C@@H](CO)C(F)F] | 235 |
| 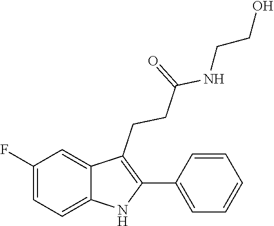 [SMILES: O=C(CCc1c(-c2ccccc2)[nH]c2ccc(F)cc12)NCCO] | 236 |
| 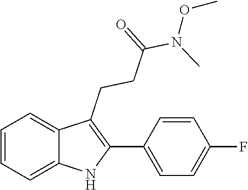 [SMILES: CON(C)C(=O)CCc1c(-c2ccc(F)cc2)[nH]c2ccccc12] | 237 |
| 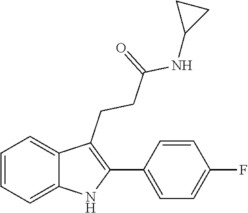 [SMILES: O=C(CCc1c(-c2ccc(F)cc2)[nH]c2ccccc12)NC1CC1] | 238 |
| 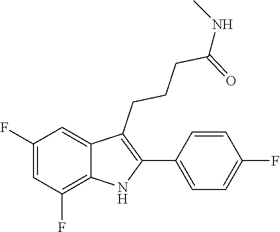 [SMILES: CNC(=O)CCCc1c(-c2ccc(F)cc2)[nH]c2c(F)cc(F)cc12] | 239 |
| 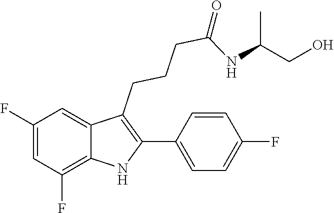 [SMILES: C[C@@H](CO)NC(=O)CCCc1c(-c2ccc(F)cc2)[nH]c2c(F)cc(F)cc12] | 240 |
| 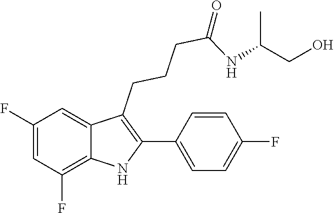 [SMILES: C[C@H](CO)NC(=O)CCCc1c(-c2ccc(F)cc2)[nH]c2c(F)cc(F)cc12] | 241 |
| 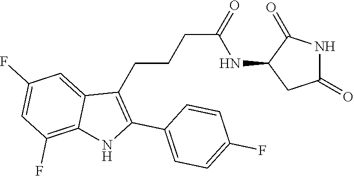 [SMILES: O=C1C[C@@H](NC(=O)CCCc2c(-c3ccc(F)cc3)[nH]c3c(F)cc(F)cc23)C(=O)N1] | 242 |
| 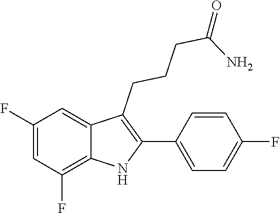 [SMILES: NC(=O)CCCc1c(-c2ccc(F)cc2)[nH]c2c(F)cc(F)cc12] | 243 |
| 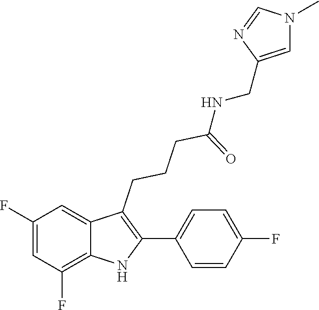 [SMILES: Cn1cnc(CNC(=O)CCCc2c(-c3ccc(F)cc3)[nH]c3c(F)cc(F)cc23)c1] | 244 |
| 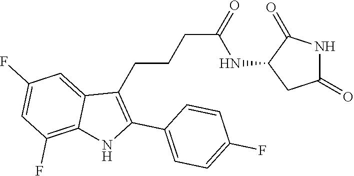 [SMILES: O=C1C[C@H](NC(=O)CCCc2c(-c3ccc(F)cc3)[nH]c3c(F)cc(F)cc23)C(=O)N1] | 245 |
| 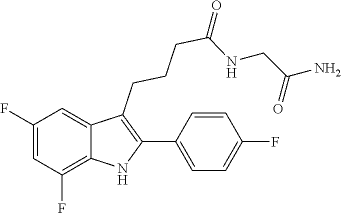 [SMILES: NC(=O)CNC(=O)CCCc1c(-c2ccc(F)cc2)[nH]c2c(F)cc(F)cc12] | 246 |
| 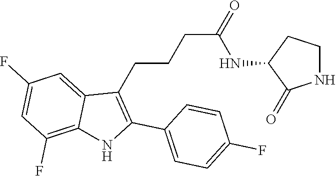 [SMILES: O=C(CCCc1c(-c2ccc(F)cc2)[nH]c2c(F)cc(F)cc12)N[C@@H]1CCNC1=O] | 247 |
| 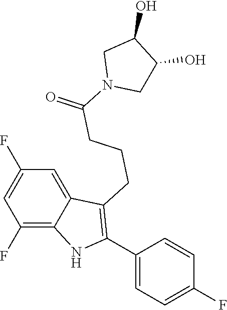 [SMILES: O=C(CCCc1c(-c2ccc(F)cc2)[nH]c2c(F)cc(F)cc12)N1C[C@@H](O)[C@H](O)C1] | 248 |
| 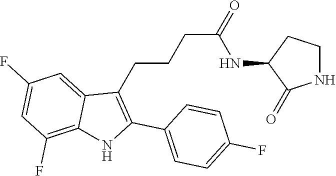 [SMILES: O=C(CCCc1c(-c2ccc(F)cc2)[nH]c2c(F)cc(F)cc12)N[C@H]1CCNC1=O] | 249 |
| 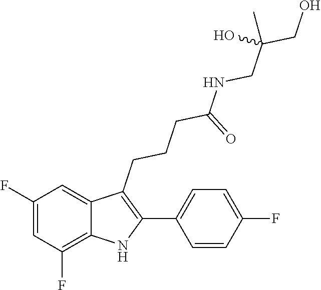 [SMILES: CC(O)(CO)CNC(=O)CCCc1c(-c2ccc(F)cc2)[nH]c2c(F)cc(F)cc12] | 250 |
| 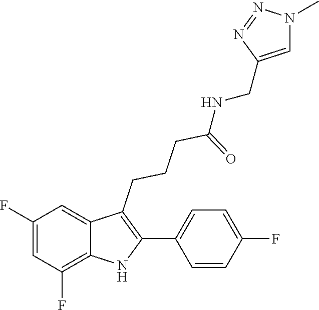 [SMILES: Cn1cc(CNC(=O)CCCc2c(-c3ccc(F)cc3)[nH]c3c(F)cc(F)cc23)nn1] | 251 |
| 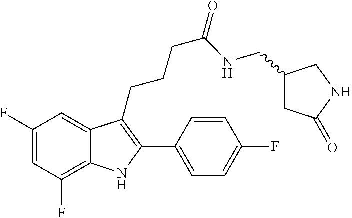 [SMILES: O=C(CCCc1c(-c2ccc(F)cc2)[nH]c2c(F)cc(F)cc12)NCC1CNC(=O)C1] | 252 |
| 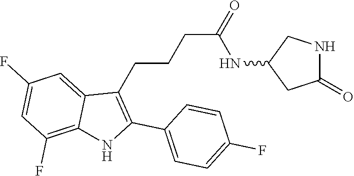 [SMILES: O=C1CC(NC(=O)CCCc2c(-c3ccc(F)cc3)[nH]c3c(F)cc(F)cc23)CN1] | 253 |
| 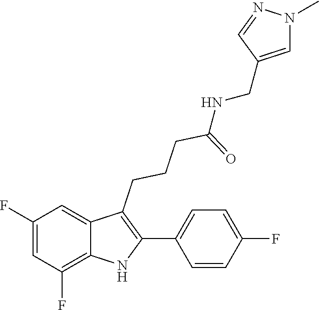 [SMILES: Cn1cc(CNC(=O)CCCc2c(-c3ccc(F)cc3)[nH]c3c(F)cc(F)cc23)cn1] | 254 |
| 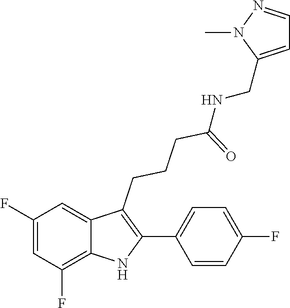 [SMILES: Cn1nccc1CNC(=O)CCCc1c(-c2ccc(F)cc2)[nH]c2c(F)cc(F)cc12] | 255 |
| 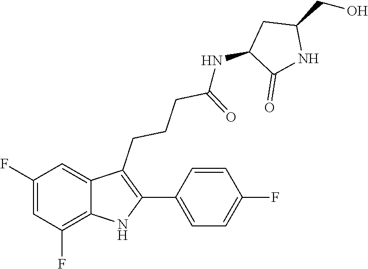 [SMILES: O=C(CCCc1c(-c2ccc(F)cc2)[nH]c2c(F)cc(F)cc12)N[C@H]1C[C@@H](CO)NC1=O] | 256 |
| 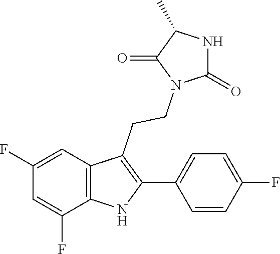 [SMILES: C[C@@H]1NC(=O)N(CCc2c(-c3ccc(F)cc3)[nH]c3c(F)cc(F)cc23)C1=O] | 257 |
| 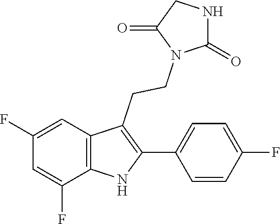 [SMILES: O=C1CNC(=O)N1CCc1c(-c2ccc(F)cc2)[nH]c2c(F)cc(F)cc12] | 258 |
| 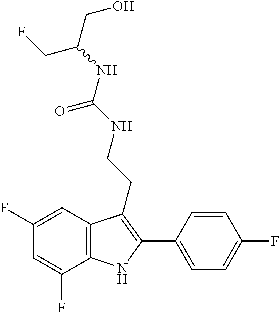 [SMILES: O=C(NCCc1c(-c2ccc(F)cc2)[nH]c2c(F)cc(F)cc12)NC(CO)CF] | 259 |
| 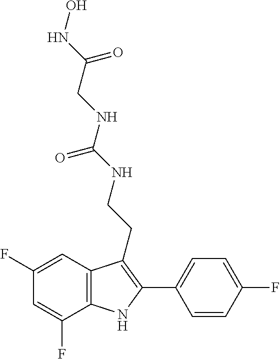 [SMILES: O=C(CNC(=O)NCCc1c(-c2ccc(F)cc2)[nH]c2c(F)cc(F)cc12)NO] | 260 |
| 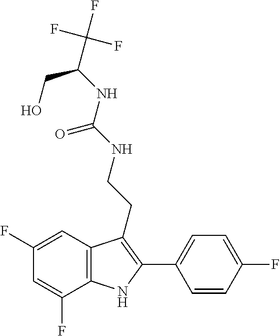 [SMILES: O=C(NCCc1c(-c2ccc(F)cc2)[nH]c2c(F)cc(F)cc12)N[C@@H](CO)C(F)(F)F] | 261 |
| 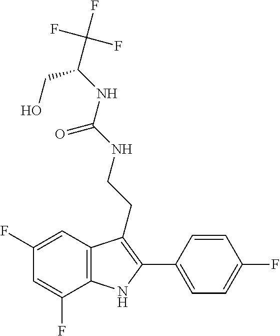 [SMILES: O=C(NCCc1c(-c2ccc(F)cc2)[nH]c2c(F)cc(F)cc12)N[C@H](CO)C(F)(F)F] | 262 |
| 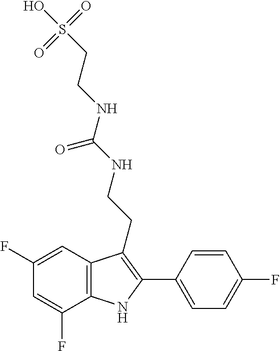 [SMILES: O=C(NCCc1c(-c2ccc(F)cc2)[nH]c2c(F)cc(F)cc12)NCCS(=O)(=O)O] | 263 |
| 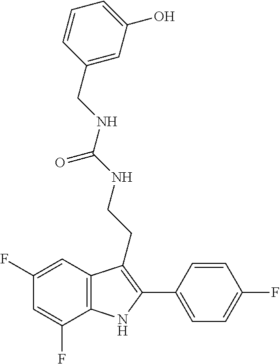 [SMILES: O=C(NCCc1c(-c2ccc(F)cc2)[nH]c2c(F)cc(F)cc12)NCc1cccc(O)c1] | 264 |
| 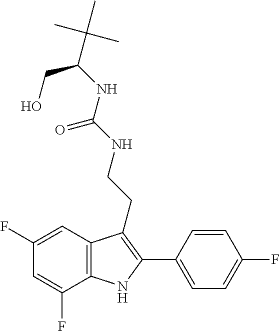 [SMILES: CC(C)(C)[C@H](CO)NC(=O)NCCc1c(-c2ccc(F)cc2)[nH]c2c(F)cc(F)cc12] | 265 |
| 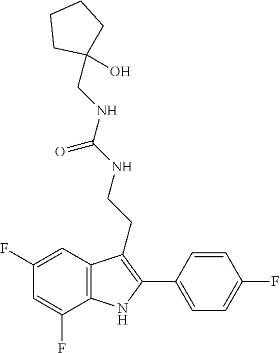 [SMILES: O=C(NCCc1c(-c2ccc(F)cc2)[nH]c2c(F)cc(F)cc12)NCC1(O)CCCC1] | 266 |
| 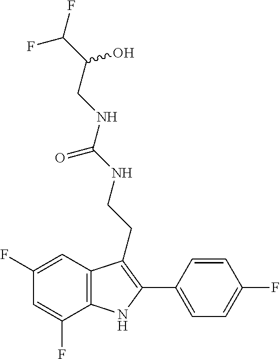 [SMILES: O=C(NCCc1c(-c2ccc(F)cc2)[nH]c2c(F)cc(F)cc12)NCC(O)C(F)F] | 267 |
| 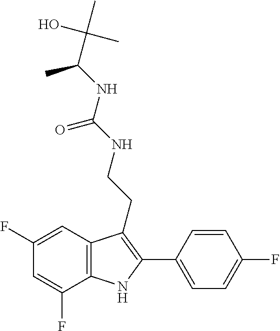 [SMILES: C[C@H](NC(=O)NCCc1c(-c2ccc(F)cc2)[nH]c2c(F)cc(F)cc12)C(C)(C)O] | 268 |
| 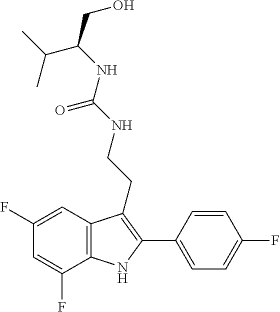 [SMILES: CC(C)[C@@H](CO)NC(=O)NCCc1c(-c2ccc(F)cc2)[nH]c2c(F)cc(F)cc12] | 269 |
| 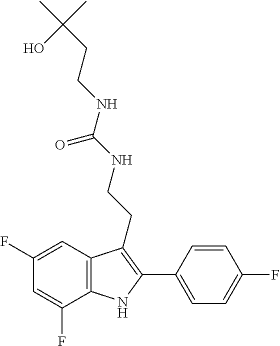 [SMILES: CC(C)(O)CCNC(=O)NCCc1c(-c2ccc(F)cc2)[nH]c2c(F)cc(F)cc12] | 270 |
| 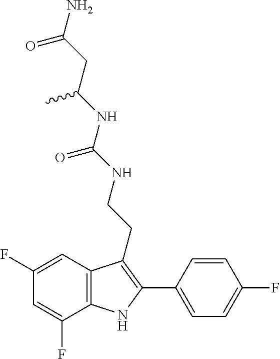 [SMILES: CC(CC(N)=O)NC(=O)NCCc1c(-c2ccc(F)cc2)[nH]c2c(F)cc(F)cc12] | 271 |
| 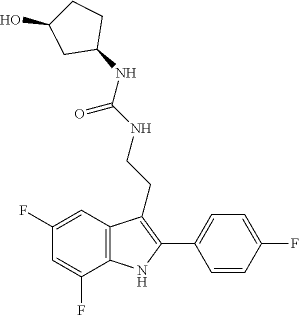 [SMILES: O=C(NCCc1c(-c2ccc(F)cc2)[nH]c2c(F)cc(F)cc12)N[C@@H]1CC[C@H](O)C1] | 272 |
| 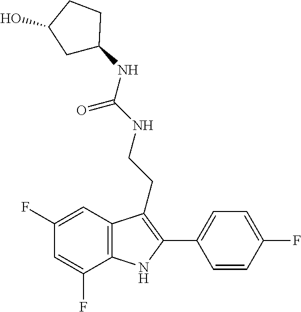 [SMILES: O=C(NCCc1c(-c2ccc(F)cc2)[nH]c2c(F)cc(F)cc12)N[C@@H]1CC[C@@H](O)C1] | 273 |
| 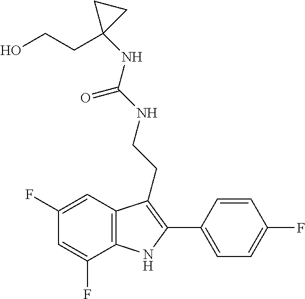 [SMILES: O=C(NCCc1c(-c2ccc(F)cc2)[nH]c2c(F)cc(F)cc12)NC1(CCO)CC1] | 274 |
| 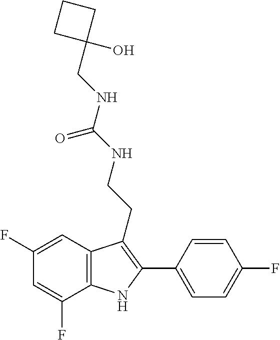 [SMILES: O=C(NCCc1c(-c2ccc(F)cc2)[nH]c2c(F)cc(F)cc12)NCC1(O)CCC1] | 275 |
| 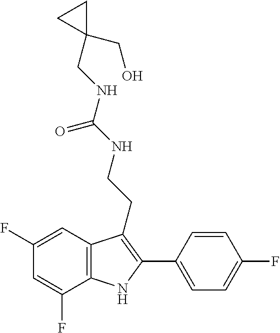 [SMILES: O=C(NCCc1c(-c2ccc(F)cc2)[nH]c2c(F)cc(F)cc12)NCC1(CO)CC1] | 276 |
| 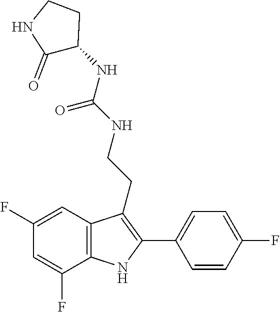 [SMILES: O=C(NCCc1c(-c2ccc(F)cc2)[nH]c2c(F)cc(F)cc12)N[C@H]1CCNC1=O] | 277 |
| 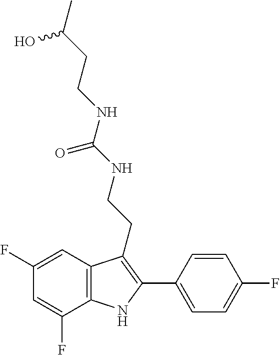 [SMILES: CC(O)CCNC(=O)NCCc1c(-c2ccc(F)cc2)[nH]c2c(F)cc(F)cc12] | 278 |
| 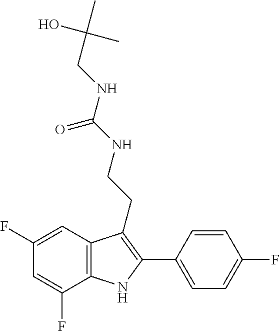 [SMILES: CC(C)(O)CNC(=O)NCCc1c(-c2ccc(F)cc2)[nH]c2c(F)cc(F)cc12] | 279 |
| 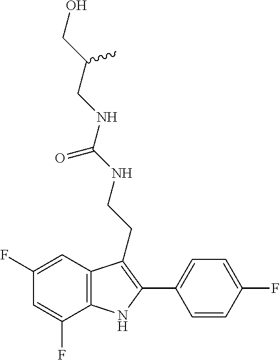 [SMILES: CC(CO)CNC(=O)NCCc1c(-c2ccc(F)cc2)[nH]c2c(F)cc(F)cc12] | 280 |
| 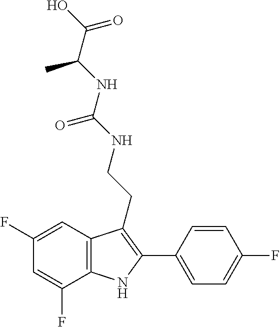 [SMILES: C[C@H](NC(=O)NCCc1c(-c2ccc(F)cc2)[nH]c2c(F)cc(F)cc12)C(=O)O] | 281 |
| 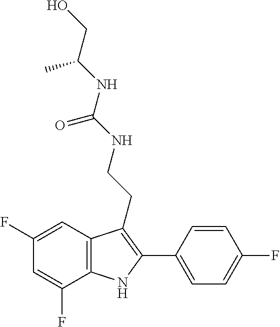 [SMILES: C[C@H](CO)NC(=O)NCCc1c(-c2ccc(F)cc2)[nH]c2c(F)cc(F)cc12] | 282 |
| 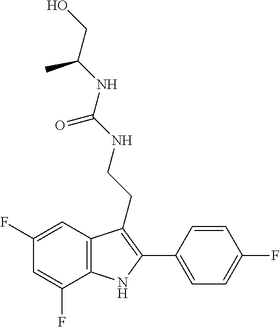 [SMILES: C[C@@H](CO)NC(=O)NCCc1c(-c2ccc(F)cc2)[nH]c2c(F)cc(F)cc12] | 283 |
| 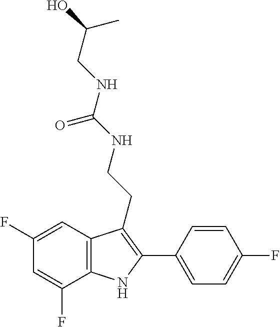 [SMILES: C[C@H](O)CNC(=O)NCCc1c(-c2ccc(F)cc2)[nH]c2c(F)cc(F)cc12] | 284 |
| 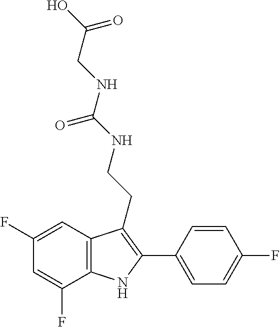 [SMILES: O=C(O)CNC(=O)NCCc1c(-c2ccc(F)cc2)[nH]c2c(F)cc(F)cc12] | 285 |
| 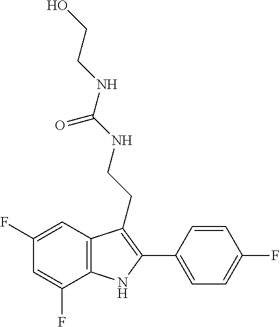 [SMILES: O=C(NCCO)NCCc1c(-c2ccc(F)cc2)[nH]c2c(F)cc(F)cc12] | 286 |
| 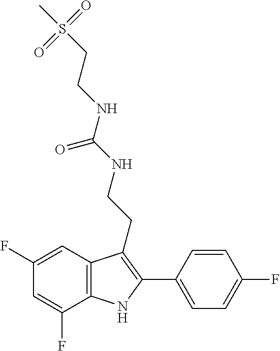 [SMILES: CS(=O)(=O)CCNC(=O)NCCc1c(-c2ccc(F)cc2)[nH]c2c(F)cc(F)cc12] | 287 |
| 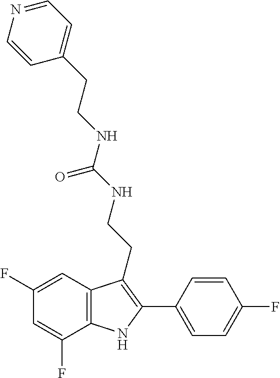 [SMILES: O=C(NCCc1ccncc1)NCCc1c(-c2ccc(F)cc2)[nH]c2c(F)cc(F)cc12] | 288 |
| 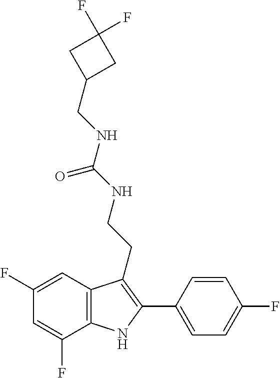 [SMILES: O=C(NCCc1c(-c2ccc(F)cc2)[nH]c2c(F)cc(F)cc12)NCC1CC(F)(F)C1] | 289 |
| 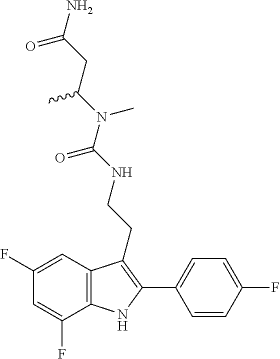 [SMILES: CC(CC(N)=O)N(C)C(=O)NCCc1c(-c2ccc(F)cc2)[nH]c2c(F)cc(F)cc12] | 290 |
| 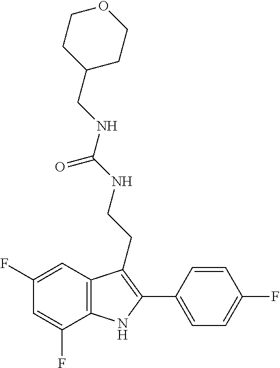 [SMILES: O=C(NCCc1c(-c2ccc(F)cc2)[nH]c2c(F)cc(F)cc12)NCC1CCOCC1] | 291 |
| 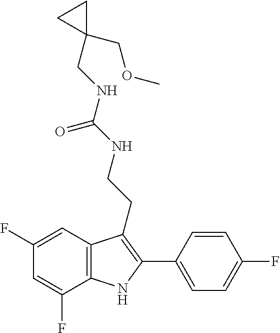 [SMILES: COCC1(CNC(=O)NCCc2c(-c3ccc(F)cc3)[nH]c3c(F)cc(F)cc23)CC1] | 292 |
| 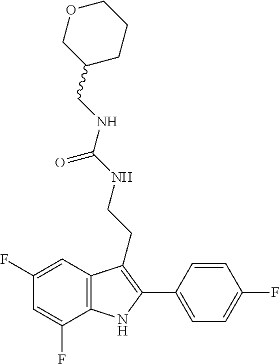 [SMILES: O=C(NCCc1c(-c2ccc(F)cc2)[nH]c2c(F)cc(F)cc12)NCC1CCCOC1] | 293 |
| 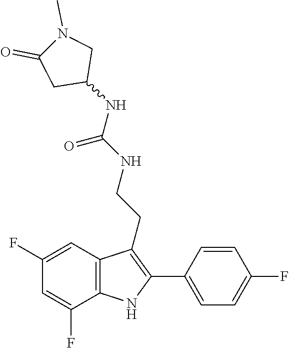 [SMILES: CN1CC(NC(=O)NCCc2c(-c3ccc(F)cc3)[nH]c3c(F)cc(F)cc23)CC1=O] | 294 |
| 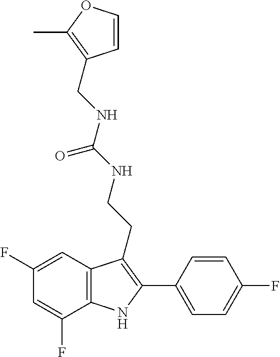 [SMILES: Cc1occc1CNC(=O)NCCc1c(-c2ccc(F)cc2)[nH]c2c(F)cc(F)cc12] | 295 |
| 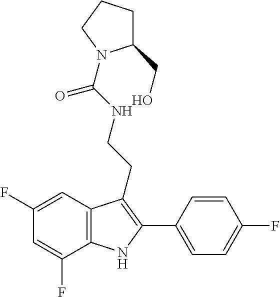 [SMILES: O=C(NCCc1c(-c2ccc(F)cc2)[nH]c2c(F)cc(F)cc12)N1CCC[C@H]1CO] | 296 |
| 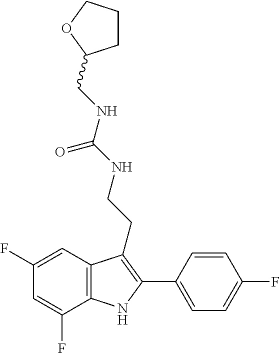 [SMILES: O=C(NCCc1c(-c2ccc(F)cc2)[nH]c2c(F)cc(F)cc12)NCC1CCCO1] | 297 |
| 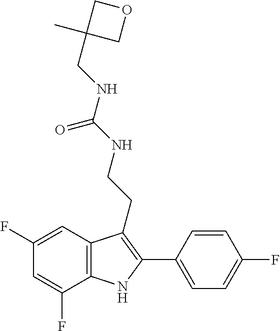 [SMILES: CC1(CNC(=O)NCCc2c(-c3ccc(F)cc3)[nH]c3c(F)cc(F)cc23)COC1] | 298 |
| 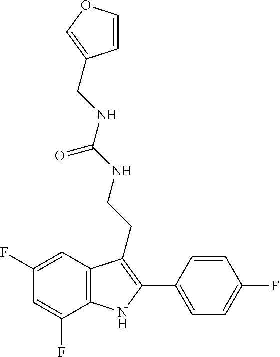 [SMILES: O=C(NCCc1c(-c2ccc(F)cc2)[nH]c2c(F)cc(F)cc12)NCc1ccoc1] | 299 |
| 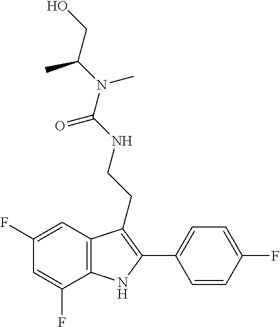 [SMILES: C[C@@H](CO)N(C)C(=O)NCCc1c(-c2ccc(F)cc2)[nH]c2c(F)cc(F)cc12] | 300 |
| 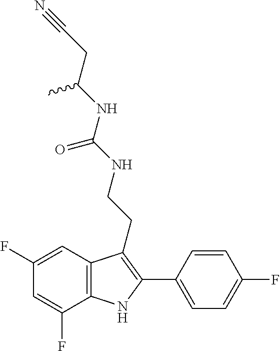 [SMILES: CC(CC#N)NC(=O)NCCc1c(-c2ccc(F)cc2)[nH]c2c(F)cc(F)cc12] | 301 |
| 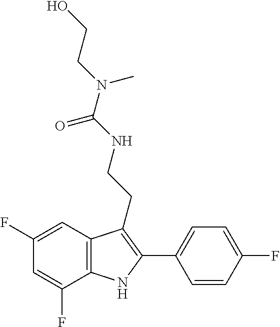 [SMILES: CN(CCO)C(=O)NCCc1c(-c2ccc(F)cc2)[nH]c2c(F)cc(F)cc12] | 302 |
| 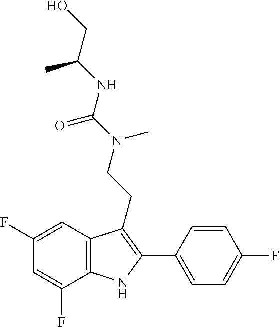 [SMILES: C[C@@H](CO)NC(=O)N(C)CCc1c(-c2ccc(F)cc2)[nH]c2c(F)cc(F)cc12] | 303 |
| 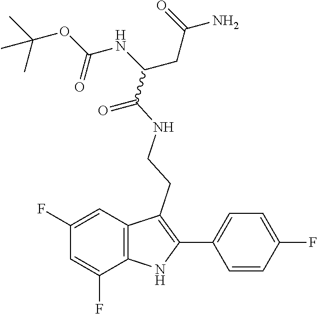 [SMILES: CC(C)(C)OC(=O)NC(CC(N)=O)C(=O)NCCc1c(-c2ccc(F)cc2)[nH]c2c(F)cc(F)cc12] | 304 |
| 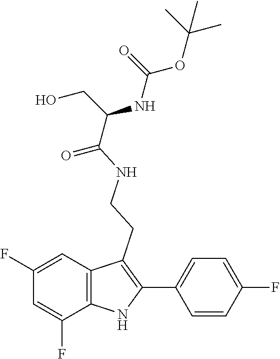 [SMILES: CC(C)(C)OC(=O)N[C@H](CO)C(=O)NCCc1c(-c2ccc(F)cc2)[nH]c2c(F)cc(F)cc12] | 305 |
| 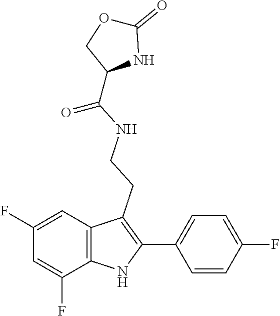 [SMILES: O=C1N[C@@H](C(=O)NCCc2c(-c3ccc(F)cc3)[nH]c3c(F)cc(F)cc23)CO1] | 306 |
| 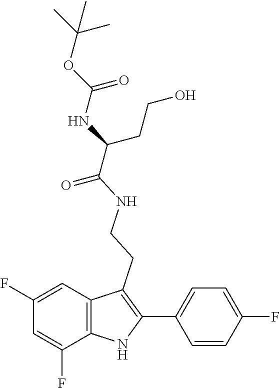 [SMILES: CC(C)(C)OC(=O)N[C@@H](CCO)C(=O)NCCc1c(-c2ccc(F)cc2)[nH]c2c(F)cc(F)cc12] | 307 |
| 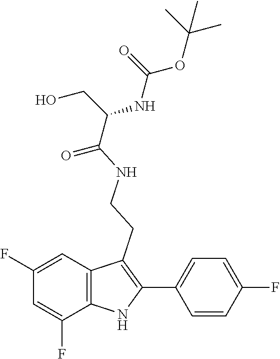 [SMILES: CC(C)(C)OC(=O)N[C@@H](CO)C(=O)NCCc1c(-c2ccc(F)cc2)[nH]c2c(F)cc(F)cc12] | 308 |
| 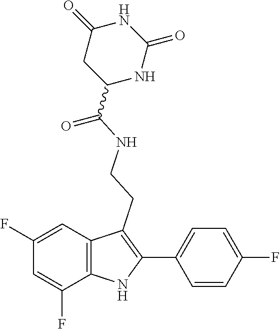 [SMILES: O=C1CC(C(=O)NCCc2c(-c3ccc(F)cc3)[nH]c3c(F)cc(F)cc23)NC(=O)N1] | 309 |
| 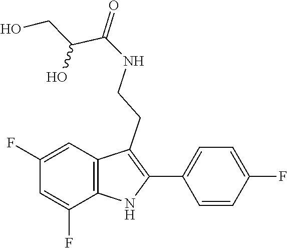 [SMILES: O=C(NCCc1c(-c2ccc(F)cc2)[nH]c2c(F)cc(F)cc12)C(O)CO] | 310 |
| 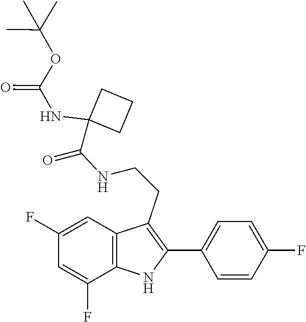 [SMILES: CC(C)(C)OC(=O)NC1(C(=O)NCCc2c(-c3ccc(F)cc3)[nH]c3c(F)cc(F)cc23)CCC1] | 311 |
| 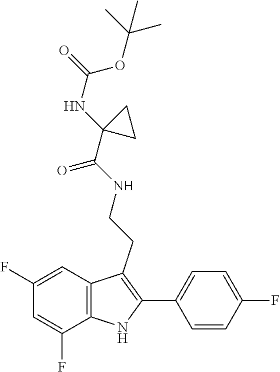 [SMILES: CC(C)(C)OC(=O)NC1(C(=O)NCCc2c(-c3ccc(F)cc3)[nH]c3c(F)cc(F)cc23)CC1] | 312 |
| 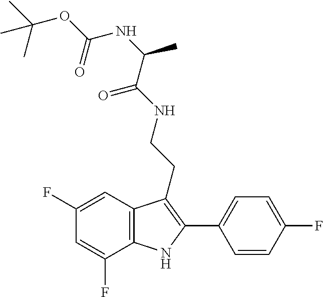 [SMILES: C[C@H](NC(=O)OC(C)(C)C)C(=O)NCCc1c(-c2ccc(F)cc2)[nH]c2c(F)cc(F)cc12] | 313 |
| 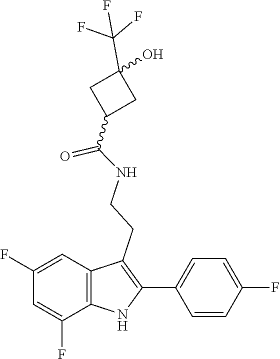 [SMILES: O=C(NCCc1c(-c2ccc(F)cc2)[nH]c2c(F)cc(F)cc12)C1CC(O)(C(F)(F)F)C1] | 314 |
| 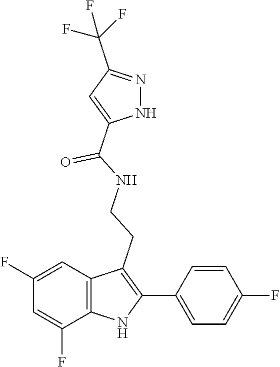 [SMILES: O=C(NCCc1c(-c2ccc(F)cc2)[nH]c2c(F)cc(F)cc12)c1cc(C(F)(F)F)n[nH]1] | 315 |
| 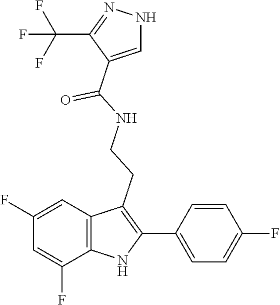 [SMILES: O=C(NCCc1c(-c2ccc(F)cc2)[nH]c2c(F)cc(F)cc12)c1c[nH]nc1C(F)(F)F] | 316 |
| 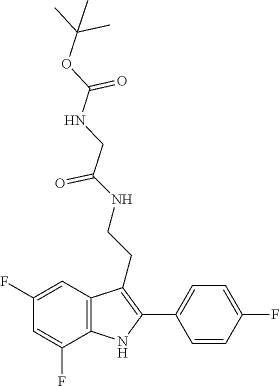 [SMILES: CC(C)(C)OC(=O)NCC(=O)NCCc1c(-c2ccc(F)cc2)[nH]c2c(F)cc(F)cc12] | 317 |
| 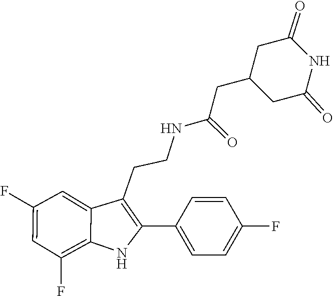 [SMILES: O=C(CC1CC(=O)NC(=O)C1)NCCc1c(-c2ccc(F)cc2)[nH]c2c(F)cc(F)cc12] | 318 |
| 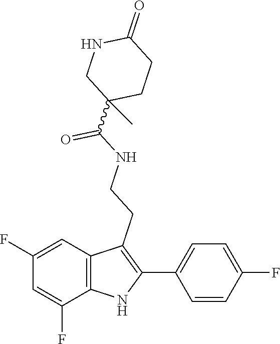 [SMILES: CC1(C(=O)NCCc2c(-c3ccc(F)cc3)[nH]c3c(F)cc(F)cc23)CCC(=O)NC1] | 319 |
| 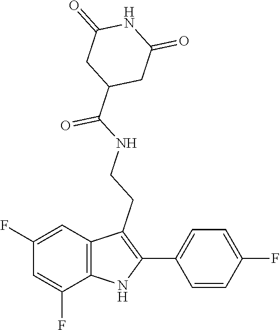 [SMILES: O=C1CC(C(=O)NCCc2c(-c3ccc(F)cc3)[nH]c3c(F)cc(F)cc23)CC(=O)N1] | 320 |
| 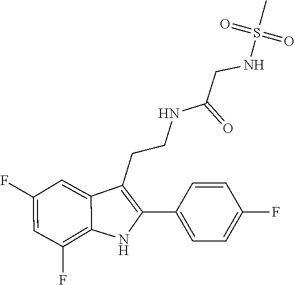 [SMILES: CS(=O)(=O)NCC(=O)NCCc1c(-c2ccc(F)cc2)[nH]c2c(F)cc(F)cc12] | 321 |
| 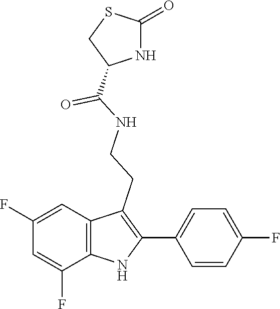 [SMILES: O=C1N[C@H](C(=O)NCCc2c(-c3ccc(F)cc3)[nH]c3c(F)cc(F)cc23)CS1] | 322 |
| 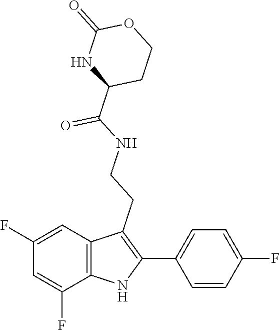 [SMILES: O=C1N[C@H](C(=O)NCCc2c(-c3ccc(F)cc3)[nH]c3c(F)cc(F)cc23)CCO1] | 323 |
| 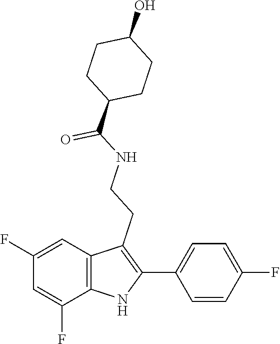 [SMILES: O=C(NCCc1c(-c2ccc(F)cc2)[nH]c2c(F)cc(F)cc12)[C@H]1CC[C@@H](O)CC1] | 324 |
| 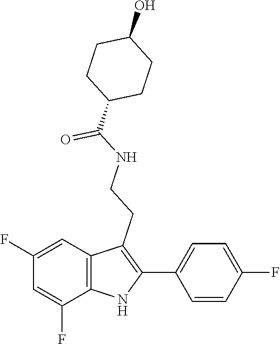 [SMILES: O=C(NCCc1c(-c2ccc(F)cc2)[nH]c2c(F)cc(F)cc12)[C@H]1CC[C@H](O)CC1] | 325 |
| 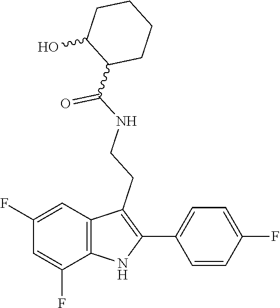 [SMILES: O=C(NCCc1c(-c2ccc(F)cc2)[nH]c2c(F)cc(F)cc12)C1CCCCC1O] | 326 |
| 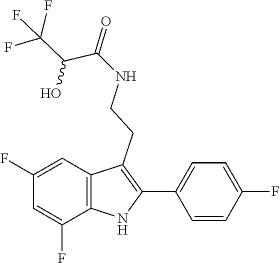 [SMILES: O=C(NCCc1c(-c2ccc(F)cc2)[nH]c2c(F)cc(F)cc12)C(O)C(F)(F)F] | 327 |
| 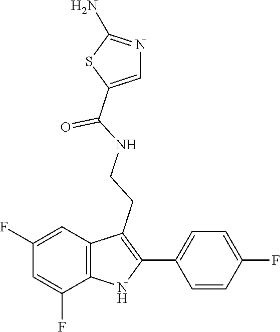 [SMILES: Nc1ncc(C(=O)NCCc2c(-c3ccc(F)cc3)[nH]c3c(F)cc(F)cc23)s1] | 328 |
| 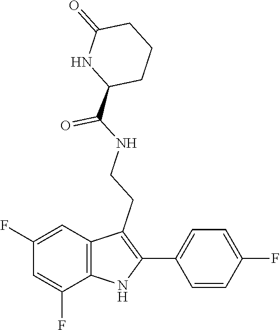 [SMILES: O=C1CCC[C@@H](C(=O)NCCc2c(-c3ccc(F)cc3)[nH]c3c(F)cc(F)cc23)N1] | 329 |
| 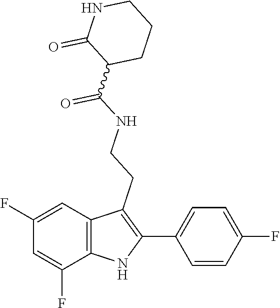 [SMILES: O=C1NCCCC1C(=O)NCCc1c(-c2ccc(F)cc2)[nH]c2c(F)cc(F)cc12] | 330 |
| 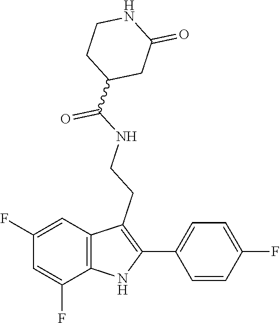 [SMILES: O=C1CC(C(=O)NCCc2c(-c3ccc(F)cc3)[nH]c3c(F)cc(F)cc23)CCN1] | 331 |
| 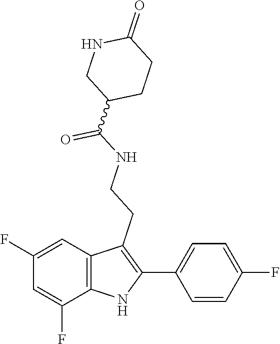 [SMILES: O=C1CCC(C(=O)NCCc2c(-c3ccc(F)cc3)[nH]c3c(F)cc(F)cc23)CN1] | 332 |
| 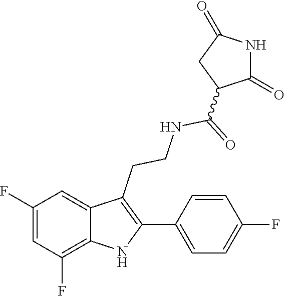 [SMILES: O=C1CC(C(=O)NCCc2c(-c3ccc(F)cc3)[nH]c3c(F)cc(F)cc23)C(=O)N1] | 333 |
| 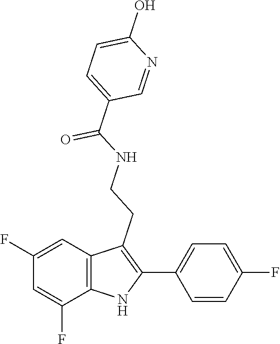 [SMILES: O=C(NCCc1c(-c2ccc(F)cc2)[nH]c2c(F)cc(F)cc12)c1ccc(O)nc1] | 334 |
| 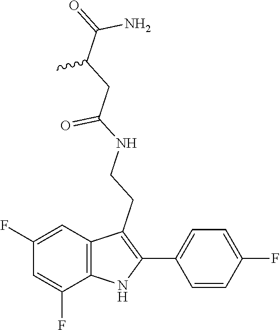 [SMILES: CC(CC(=O)NCCc1c(-c2ccc(F)cc2)[nH]c2c(F)cc(F)cc12)C(N)=O] | 335 |
| 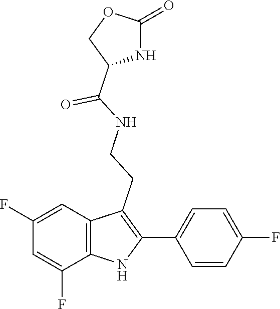 [SMILES: O=C1N[C@H](C(=O)NCCc2c(-c3ccc(F)cc3)[nH]c3c(F)cc(F)cc23)CO1] | 336 |
| 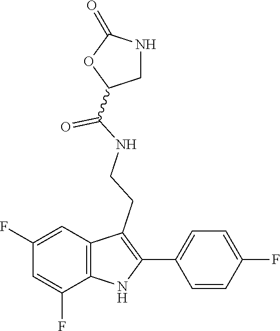 [SMILES: O=C1NCC(C(=O)NCCc2c(-c3ccc(F)cc3)[nH]c3c(F)cc(F)cc23)O1] | 337 |
| 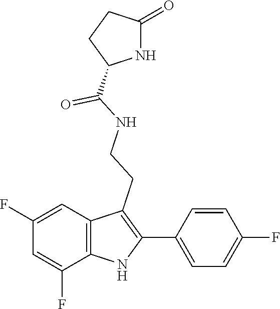 [SMILES: O=C1CC[C@@H](C(=O)NCCc2c(-c3ccc(F)cc3)[nH]c3c(F)cc(F)cc23)N1] | 338 |
| 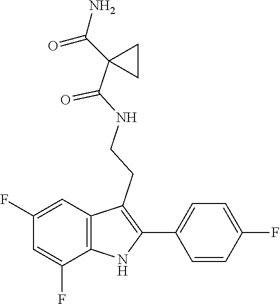 [SMILES: NC(=O)C1(C(=O)NCCc2c(-c3ccc(F)cc3)[nH]c3c(F)cc(F)cc23)CC1] | 339 |
| 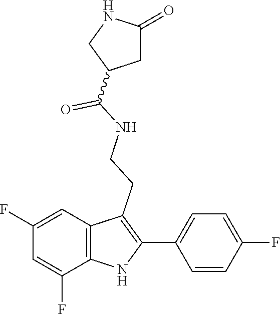 [SMILES: O=C1CC(C(=O)NCCc2c(-c3ccc(F)cc3)[nH]c3c(F)cc(F)cc23)CN1] | 340 |
| 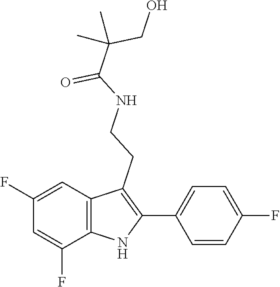 [SMILES: CC(C)(CO)C(=O)NCCc1c(-c2ccc(F)cc2)[nH]c2c(F)cc(F)cc12] | 341 |
| 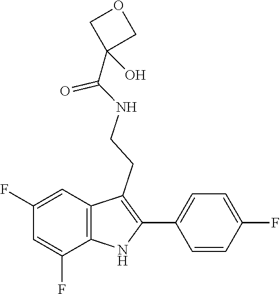 [SMILES: O=C(NCCc1c(-c2ccc(F)cc2)[nH]c2c(F)cc(F)cc12)C1(O)COC1] | 342 |
| 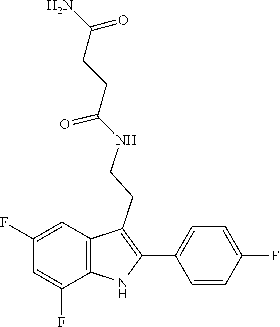 [SMILES: NC(=O)CCC(=O)NCCc1c(-c2ccc(F)cc2)[nH]c2c(F)cc(F)cc12] | 343 |
| 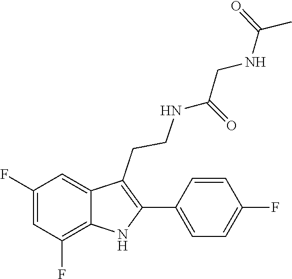 [SMILES: CC(=O)NCC(=O)NCCc1c(-c2ccc(F)cc2)[nH]c2c(F)cc(F)cc12] | 344 |
| 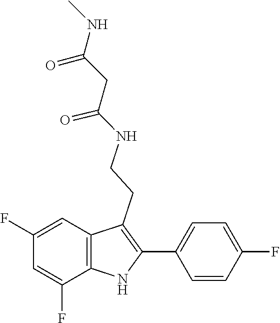 [SMILES: CNC(=O)CC(=O)NCCc1c(-c2ccc(F)cc2)[nH]c2c(F)cc(F)cc12] | 345 |
| 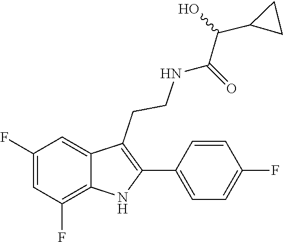 [SMILES: O=C(NCCc1c(-c2ccc(F)cc2)[nH]c2c(F)cc(F)cc12)C(O)C1CC1] | 346 |
| 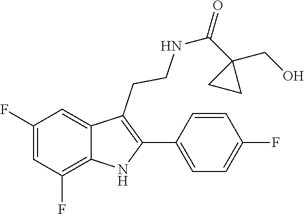 [SMILES: O=C(NCCc1c(-c2ccc(F)cc2)[nH]c2c(F)cc(F)cc12)C1(CO)CC1] | 347 |
| 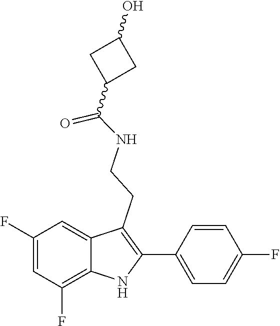 [SMILES: O=C(NCCc1c(-c2ccc(F)cc2)[nH]c2c(F)cc(F)cc12)C1CC(O)C1] | 348 |
| 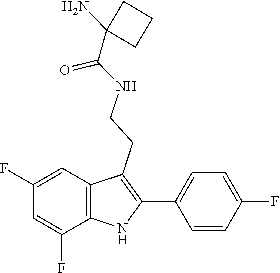 [SMILES: NC1(C(=O)NCCc2c(-c3ccc(F)cc3)[nH]c3c(F)cc(F)cc23)CCC1] | 349 |
| 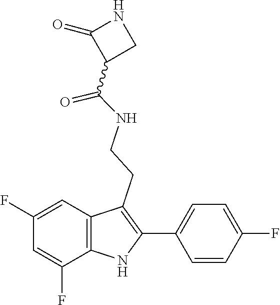 [SMILES: O=C(NCCc1c(-c2ccc(F)cc2)[nH]c2c(F)cc(F)cc12)C1CNC1=O] | 350 |
| 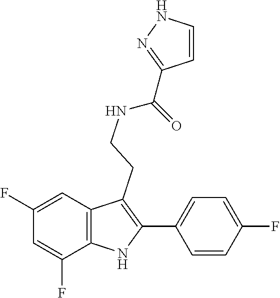 [SMILES: O=C(NCCc1c(-c2ccc(F)cc2)[nH]c2c(F)cc(F)cc12)c1cc[nH]n1] | 351 |
| 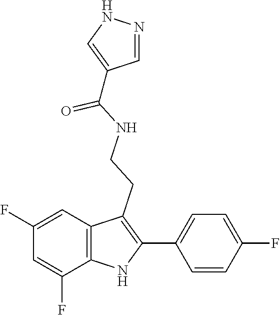 [SMILES: O=C(NCCc1c(-c2ccc(F)cc2)[nH]c2c(F)cc(F)cc12)c1cn[nH]c1] | 352 |
| 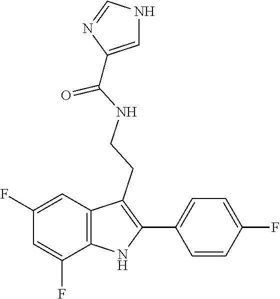 [SMILES: O=C(NCCc1c(-c2ccc(F)cc2)[nH]c2c(F)cc(F)cc12)c1c[nH]cn1] | 353 |
| 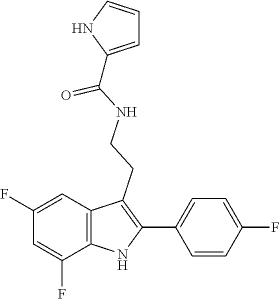 [SMILES: O=C(NCCc1c(-c2ccc(F)cc2)[nH]c2c(F)cc(F)cc12)c1ccc[nH]1] | 354 |
| 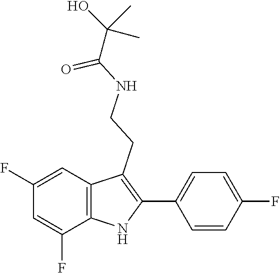 [SMILES: CC(C)(O)C(=O)NCCc1c(-c2ccc(F)cc2)[nH]c2c(F)cc(F)cc12] | 355 |
| 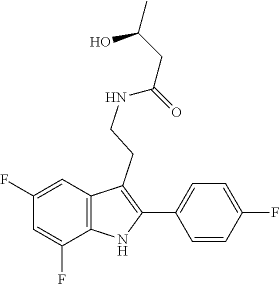 [SMILES: C[C@H](O)CC(=O)NCCc1c(-c2ccc(F)cc2)[nH]c2c(F)cc(F)cc12] | 356 |
| 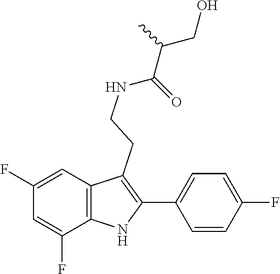 [SMILES: CC(CO)C(=O)NCCc1c(-c2ccc(F)cc2)[nH]c2c(F)cc(F)cc12] | 357 |
| 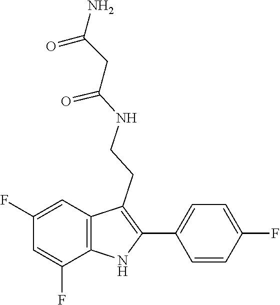 [SMILES: NC(=O)CC(=O)NCCc1c(-c2ccc(F)cc2)[nH]c2c(F)cc(F)cc12] | 358 |
| 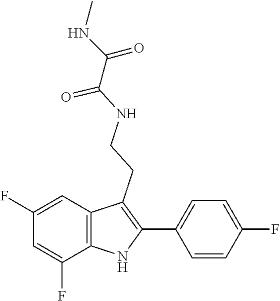 [SMILES: CNC(=O)C(=O)NCCc1c(-c2ccc(F)cc2)[nH]c2c(F)cc(F)cc12] | 359 |
| 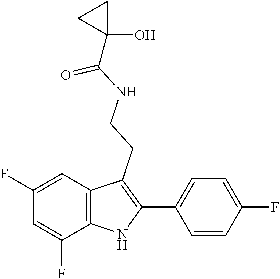 [SMILES: O=C(NCCc1c(-c2ccc(F)cc2)[nH]c2c(F)cc(F)cc12)C1(O)CC1] | 360 |
| 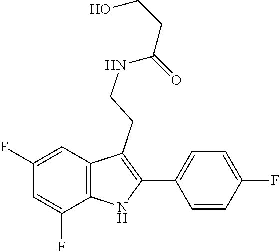 [SMILES: O=C(CCO)NCCc1c(-c2ccc(F)cc2)[nH]c2c(F)cc(F)cc12] | 361 |
| 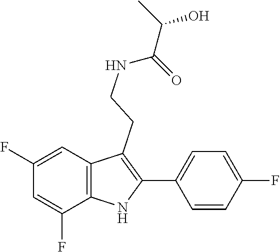 [SMILES: C[C@H](O)C(=O)NCCc1c(-c2ccc(F)cc2)[nH]c2c(F)cc(F)cc12] | 362 |
| 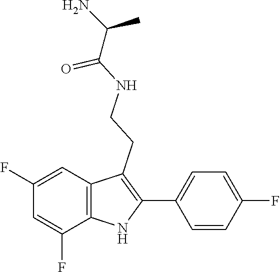 [SMILES: C[C@H](N)C(=O)NCCc1c(-c2ccc(F)cc2)[nH]c2c(F)cc(F)cc12] | 363 |
| 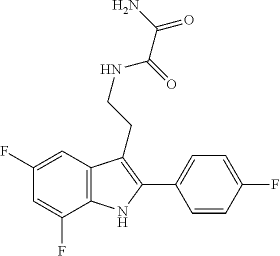 [SMILES: NC(=O)C(=O)NCCc1c(-c2ccc(F)cc2)[nH]c2c(F)cc(F)cc12] | 364 |
| 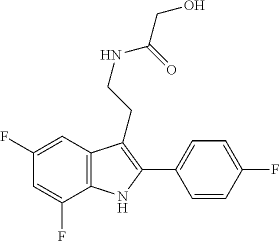 [SMILES: O=C(CO)NCCc1c(-c2ccc(F)cc2)[nH]c2c(F)cc(F)cc12] | 365 |
| 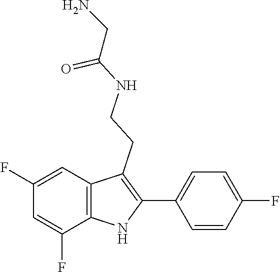 [SMILES: NCC(=O)NCCc1c(-c2ccc(F)cc2)[nH]c2c(F)cc(F)cc12] | 366 |
| 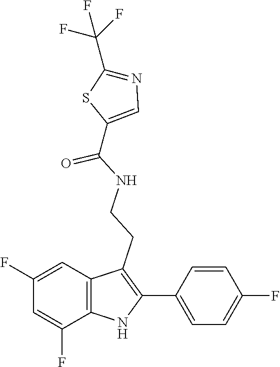 [SMILES: O=C(NCCc1c(-c2ccc(F)cc2)[nH]c2c(F)cc(F)cc12)c1cnc(C(F)(F)F)s1] | 367 |
| 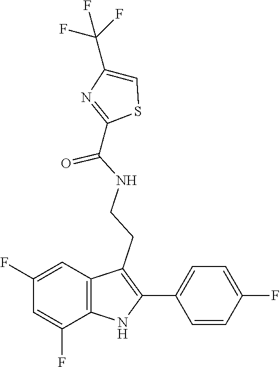 [SMILES: O=C(NCCc1c(-c2ccc(F)cc2)[nH]c2c(F)cc(F)cc12)c1nc(C(F)(F)F)cs1] | 368 |
| 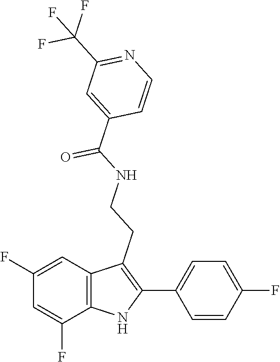 [SMILES: O=C(NCCc1c(-c2ccc(F)cc2)[nH]c2c(F)cc(F)cc12)c1ccnc(C(F)(F)F)c1] | 369 |
| 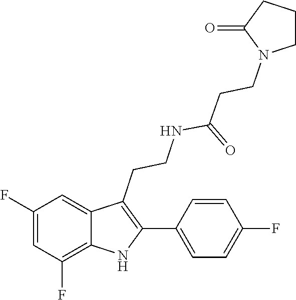 [SMILES: O=C(CCN1CCCC1=O)NCCc1c(-c2ccc(F)cc2)[nH]c2c(F)cc(F)cc12] | 370 |
| 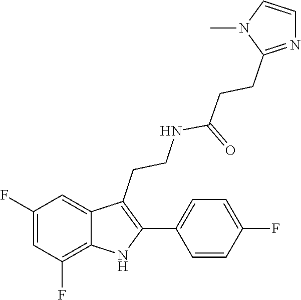 [SMILES: Cn1ccnc1CCC(=O)NCCc1c(-c2ccc(F)cc2)[nH]c2c(F)cc(F)cc12] | 371 |
| 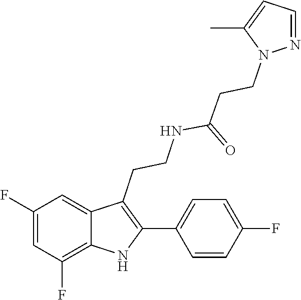 [SMILES: Cc1ccnn1CCC(=O)NCCc1c(-c2ccc(F)cc2)[nH]c2c(F)cc(F)cc12] | 372 |
| 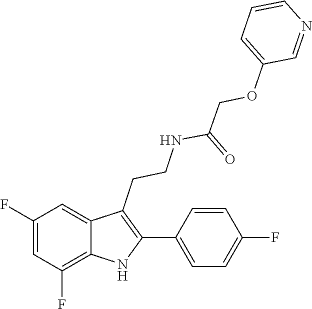 [SMILES: O=C(COc1cccnc1)NCCc1c(-c2ccc(F)cc2)[nH]c2c(F)cc(F)cc12] | 373 |
| 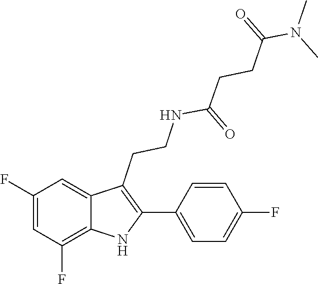 [SMILES: CN(C)C(=O)CCC(=O)NCCc1c(-c2ccc(F)cc2)[nH]c2c(F)cc(F)cc12] | 374 |
| 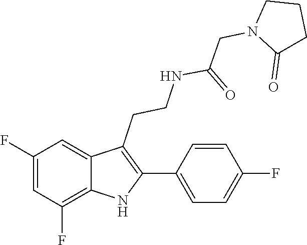 [SMILES: O=C(CN1CCCC1=O)NCCc1c(-c2ccc(F)cc2)[nH]c2c(F)cc(F)cc12] | 375 |
| 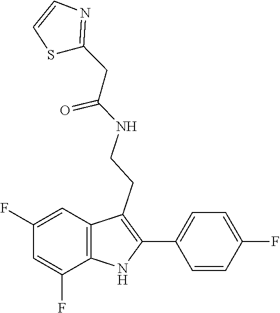 [SMILES: O=C(Cc1nccs1)NCCc1c(-c2ccc(F)cc2)[nH]c2c(F)cc(F)cc12] | 376 |
| 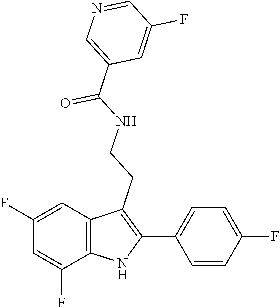 [SMILES: O=C(NCCc1c(-c2ccc(F)cc2)[nH]c2c(F)cc(F)cc12)c1cncc(F)c1] | 377 |
| 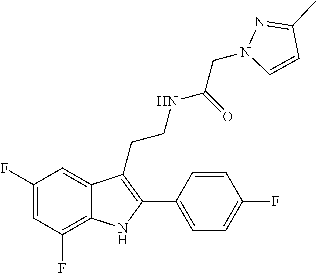 [SMILES: Cc1ccn(CC(=O)NCCc2c(-c3ccc(F)cc3)[nH]c3c(F)cc(F)cc23)n1] | 378 |
| 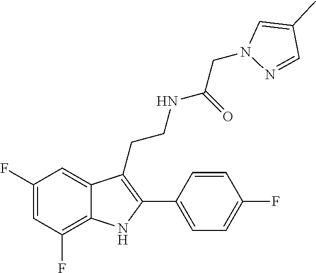 [SMILES: Cc1cnn(CC(=O)NCCc2c(-c3ccc(F)cc3)[nH]c3c(F)cc(F)cc23)c1] | 379 |
| 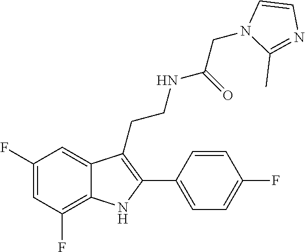 [SMILES: Cc1nccn1CC(=O)NCCc1c(-c2ccc(F)cc2)[nH]c2c(F)cc(F)cc12] | 380 |
| 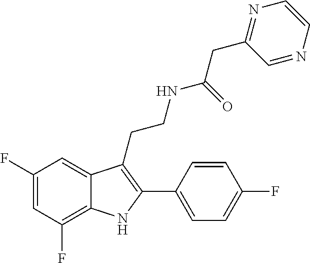 [SMILES: O=C(Cc1cnccn1)NCCc1c(-c2ccc(F)cc2)[nH]c2c(F)cc(F)cc12] | 381 |
| 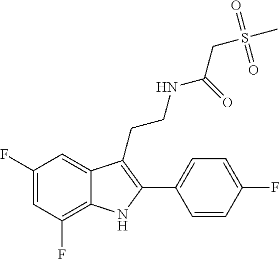 [SMILES: CS(=O)(=O)CC(=O)NCCc1c(-c2ccc(F)cc2)[nH]c2c(F)cc(F)cc12] | 382 |
| 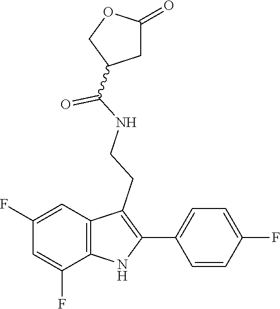 [SMILES: O=C1CC(C(=O)NCCc2c(-c3ccc(F)cc3)[nH]c3c(F)cc(F)cc23)CO1] | 383 |
| 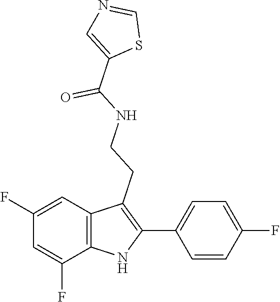 [SMILES: O=C(NCCc1c(-c2ccc(F)cc2)[nH]c2c(F)cc(F)cc12)c1cncs1] | 384 |
| 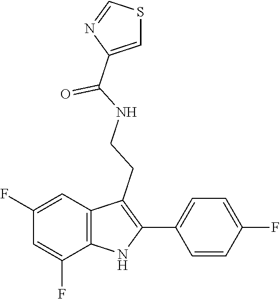 [SMILES: O=C(NCCc1c(-c2ccc(F)cc2)[nH]c2c(F)cc(F)cc12)c1cscn1] | 385 |
| 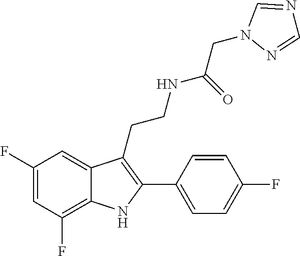 [SMILES: O=C(Cn1cncn1)NCCc1c(-c2ccc(F)cc2)[nH]c2c(F)cc(F)cc12] | 386 |
| 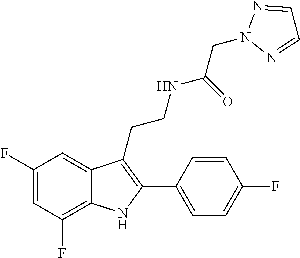 [SMILES: O=C(Cn1nccn1)NCCc1c(-c2ccc(F)cc2)[nH]c2c(F)cc(F)cc12] | 387 |
| 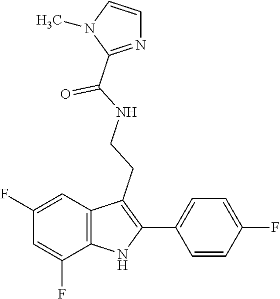 [SMILES: Cn1ccnc1C(=O)NCCc1c(-c2ccc(F)cc2)[nH]c2c(F)cc(F)cc12] | 388 |
| 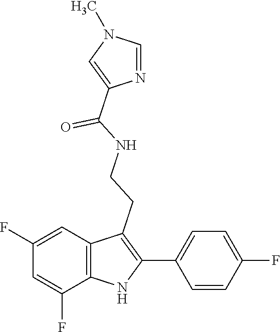 [SMILES: Cn1cnc(C(=O)NCCc2c(-c3ccc(F)cc3)[nH]c3c(F)cc(F)cc23)c1] | 389 |
| 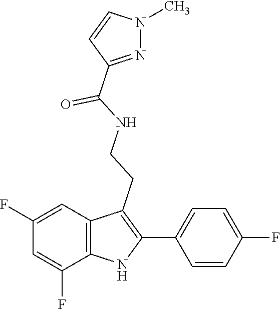 [SMILES: Cn1ccc(C(=O)NCCc2c(-c3ccc(F)cc3)[nH]c3c(F)cc(F)cc23)n1] | 390 |
| 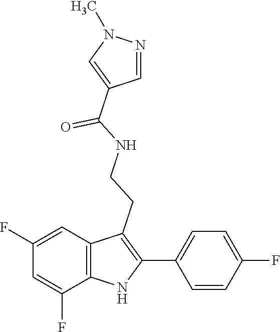 [SMILES: Cn1cc(C(=O)NCCc2c(-c3ccc(F)cc3)[nH]c3c(F)cc(F)cc23)cn1] | 391 |
| 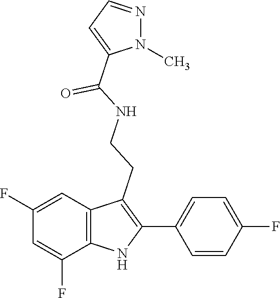 [SMILES: Cn1nccc1C(=O)NCCc1c(-c2ccc(F)cc2)[nH]c2c(F)cc(F)cc12] | 392 |
| 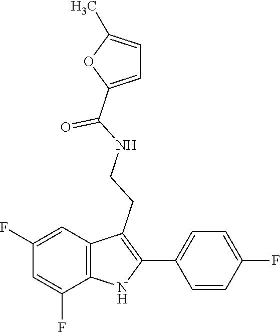 [SMILES: Cc1ccc(C(=O)NCCc2c(-c3ccc(F)cc3)[nH]c3c(F)cc(F)cc23)o1] | 393 |
| 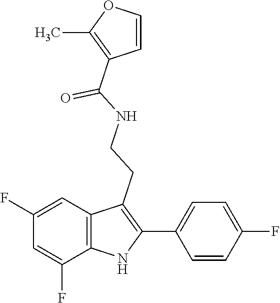 [SMILES: Cc1occc1C(=O)NCCc1c(-c2ccc(F)cc2)[nH]c2c(F)cc(F)cc12] | 394 |
| 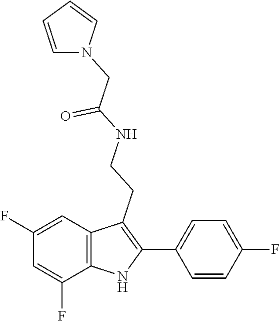 [SMILES: O=C(Cn1cccc1)NCCc1c(-c2ccc(F)cc2)[nH]c2c(F)cc(F)cc12] | 395 |
| 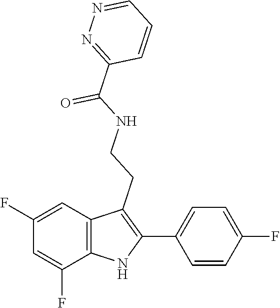 [SMILES: O=C(NCCc1c(-c2ccc(F)cc2)[nH]c2c(F)cc(F)cc12)c1cccnn1] | 396 |
| 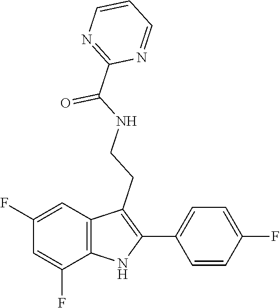 [SMILES: O=C(NCCc1c(-c2ccc(F)cc2)[nH]c2c(F)cc(F)cc12)c1ncccn1] | 397 |
| 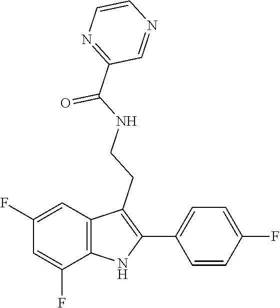 [SMILES: O=C(NCCc1c(-c2ccc(F)cc2)[nH]c2c(F)cc(F)cc12)c1cnccn1] | 398 |
| 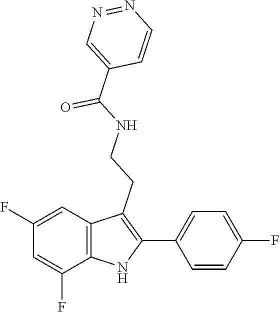 [SMILES: O=C(NCCc1c(-c2ccc(F)cc2)[nH]c2c(F)cc(F)cc12)c1ccnnc1] | 399 |
| 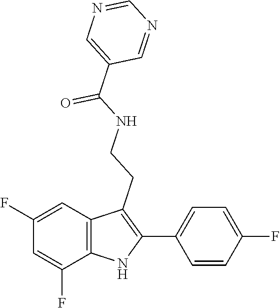 [SMILES: O=C(NCCc1c(-c2ccc(F)cc2)[nH]c2c(F)cc(F)cc12)c1cncnc1] | 400 |
| 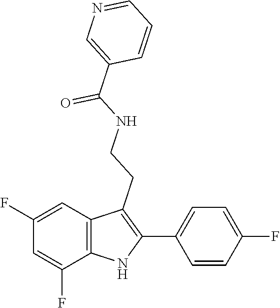 [SMILES: O=C(NCCc1c(-c2ccc(F)cc2)[nH]c2c(F)cc(F)cc12)c1cccnc1] | 401 |
| 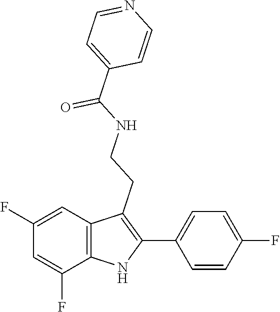 [SMILES: O=C(NCCc1c(-c2ccc(F)cc2)[nH]c2c(F)cc(F)cc12)c1ccncc1] | 402 |
| 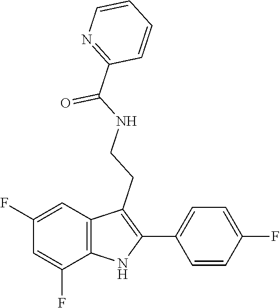 [SMILES: O=C(NCCc1c(-c2ccc(F)cc2)[nH]c2c(F)cc(F)cc12)c1ccccn1] | 403 |
| 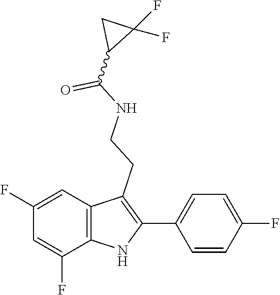 [SMILES: O=C(NCCc1c(-c2ccc(F)cc2)[nH]c2c(F)cc(F)cc12)C1CC1(F)F] | 404 |
| 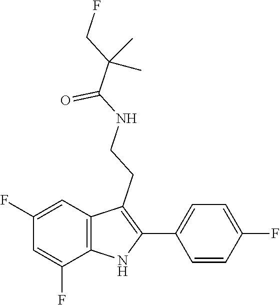 [SMILES: CC(C)(CF)C(=O)NCCc1c(-c2ccc(F)cc2)[nH]c2c(F)cc(F)cc12] | 405 |
| 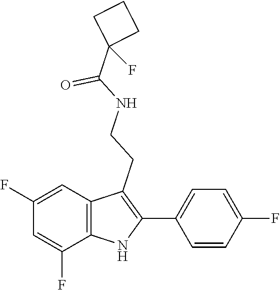 [SMILES: O=C(NCCc1c(-c2ccc(F)cc2)[nH]c2c(F)cc(F)cc12)C1(F)CCC1] | 406 |
| 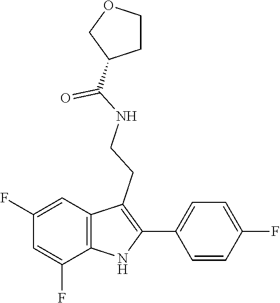 [SMILES: O=C(NCCc1c(-c2ccc(F)cc2)[nH]c2c(F)cc(F)cc12)[C@H]1CCOC1] | 407 |
| 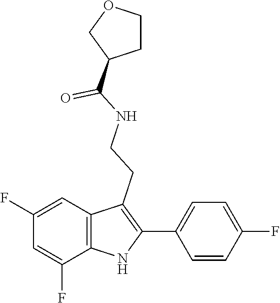 [SMILES: O=C(NCCc1c(-c2ccc(F)cc2)[nH]c2c(F)cc(F)cc12)[C@@H]1CCOC1] | 408 |
| 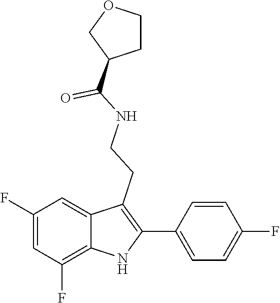 [SMILES: O=C(NCCc1c(-c2ccc(F)cc2)[nH]c2c(F)cc(F)cc12)[C@@H]1CCOC1] | 409 |
| 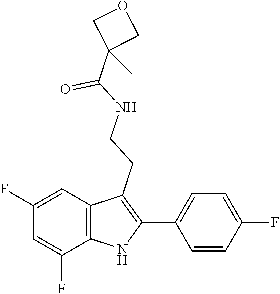 [SMILES: CC1(C(=O)NCCc2c(-c3ccc(F)cc3)[nH]c3c(F)cc(F)cc23)COC1] | 410 |
| 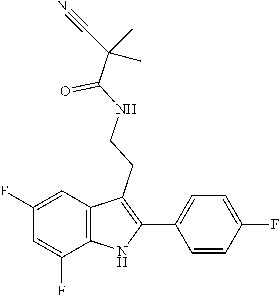 [SMILES: CC(C)(C#N)C(=O)NCCc1c(-c2ccc(F)cc2)[nH]c2c(F)cc(F)cc12] | 411 |
| 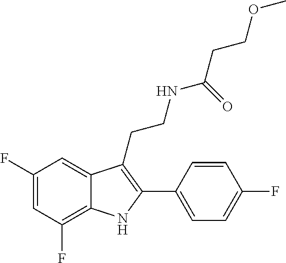 [SMILES: COCCC(=O)NCCc1c(-c2ccc(F)cc2)[nH]c2c(F)cc(F)cc12] | 412 |
| 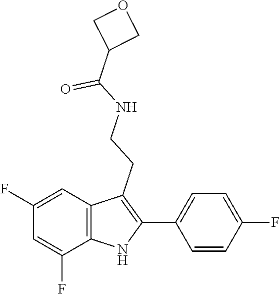 [SMILES: O=C(NCCc1c(-c2ccc(F)cc2)[nH]c2c(F)cc(F)cc12)C1COC1] | 413 |
| 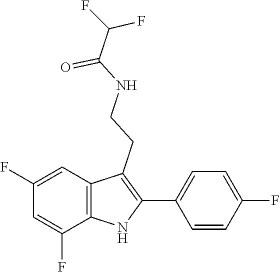 [SMILES: O=C(NCCc1c(-c2ccc(F)cc2)[nH]c2c(F)cc(F)cc12)C(F)F] | 414 |
| 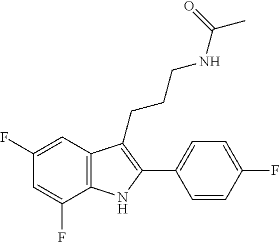 [SMILES: CC(=O)NCCCc1c(-c2ccc(F)cc2)[nH]c2c(F)cc(F)cc12] | 415 |
| 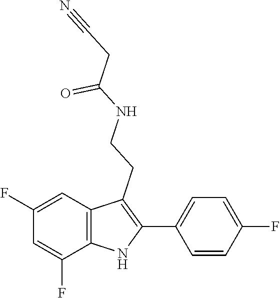 [SMILES: N#CCC(=O)NCCc1c(-c2ccc(F)cc2)[nH]c2c(F)cc(F)cc12] | 416 |
| 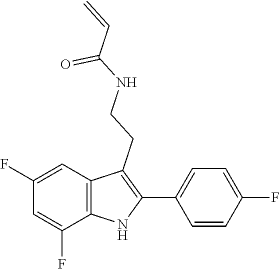 [SMILES: C=CC(=O)NCCc1c(-c2ccc(F)cc2)[nH]c2c(F)cc(F)cc12] | 417 |
| 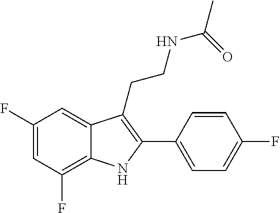 [SMILES: CC(=O)NCCc1c(-c2ccc(F)cc2)[nH]c2c(F)cc(F)cc12] | 418 |
| 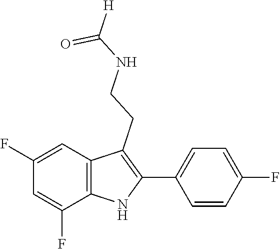 [SMILES: O=CNCCc1c(-c2ccc(F)cc2)[nH]c2c(F)cc(F)cc12] | 419 |
| 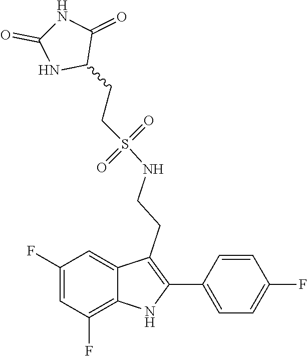 [SMILES: O=C1NC(=O)C(CCS(=O)(=O)NCCc2c(-c3ccc(F)cc3)[nH]c3c(F)cc(F)cc23)N1] | 420 |
| 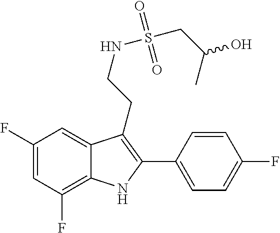 [SMILES: CC(O)CS(=O)(=O)NCCc1c(-c2ccc(F)cc2)[nH]c2c(F)cc(F)cc12] | 421 |
| 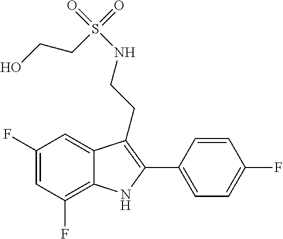 [SMILES: O=S(=O)(CCO)NCCc1c(-c2ccc(F)cc2)[nH]c2c(F)cc(F)cc12] | 422 |
| 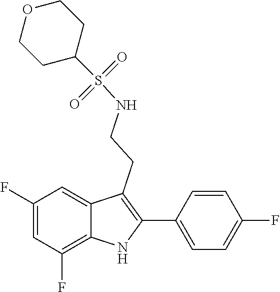 [SMILES: O=S(=O)(NCCc1c(-c2ccc(F)cc2)[nH]c2c(F)cc(F)cc12)C1CCOCC1] | 423 |
| 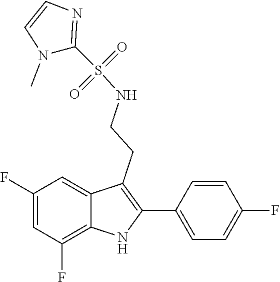 [SMILES: Cn1ccnc1S(=O)(=O)NCCc1c(-c2ccc(F)cc2)[nH]c2c(F)cc(F)cc12] | 424 |
| 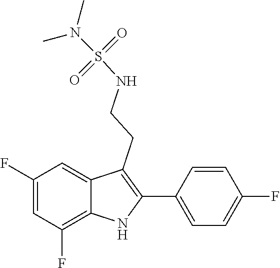 [SMILES: CN(C)S(=O)(=O)NCCc1c(-c2ccc(F)cc2)[nH]c2c(F)cc(F)cc12] | 425 |
| 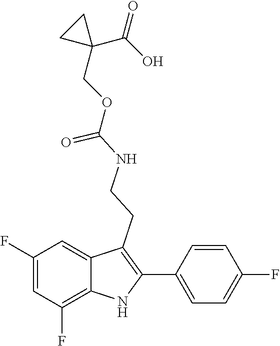 [SMILES: O=C(NCCc1c(-c2ccc(F)cc2)[nH]c2c(F)cc(F)cc12)OCC1(C(=O)O)CC1] | 426 |
| 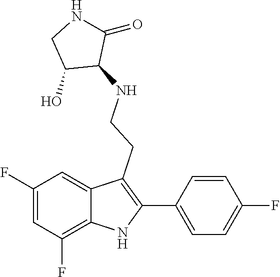 [SMILES: O=C1NC[C@@H](O)[C@@H]1NCCc1c(-c2ccc(F)cc2)[nH]c2c(F)cc(F)cc12] | 427 |
| 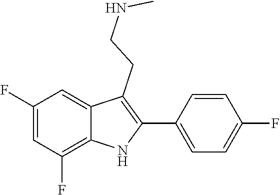 [SMILES: CNCCc1c(-c2ccc(F)cc2)[nH]c2c(F)cc(F)cc12] | 428 |
| 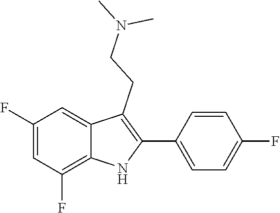 [SMILES: CN(C)CCc1c(-c2ccc(F)cc2)[nH]c2c(F)cc(F)cc12] | 429 |
| 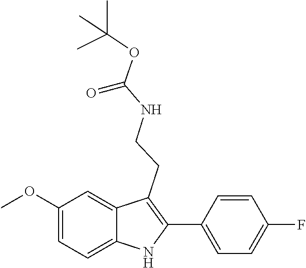 [SMILES: COc1ccc2[nH]c(-c3ccc(F)cc3)c(CCNC(=O)OC(C)(C)C)c2c1] | 430 |
| 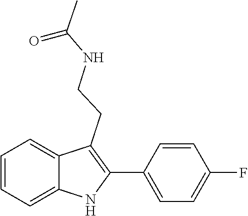 [SMILES: CC(=O)NCCc1c(-c2ccc(F)cc2)[nH]c2ccccc12] | 431 |
| 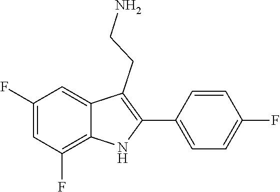 [SMILES: NCCc1c(-c2ccc(F)cc2)[nH]c2c(F)cc(F)cc12] | 432 |
| 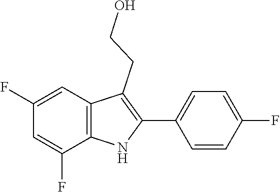 [SMILES: OCCc1c(-c2ccc(F)cc2)[nH]c2c(F)cc(F)cc12] | 433 |
| 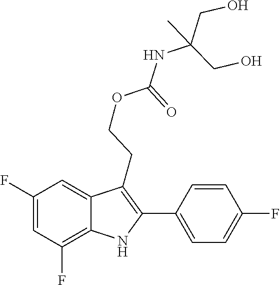 [SMILES: CC(CO)(CO)NC(=O)OCCc1c(-c2ccc(F)cc2)[nH]c2c(F)cc(F)cc12] | 434 |
| 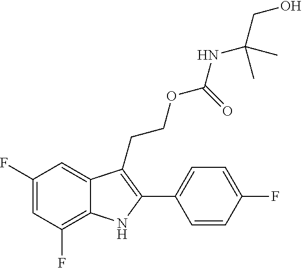 [SMILES: CC(C)(CO)NC(=O)OCCc1c(-c2ccc(F)cc2)[nH]c2c(F)cc(F)cc12] | 435 |
| 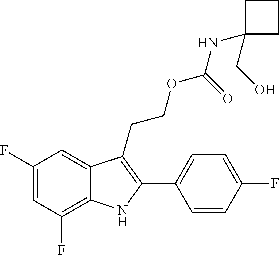 [SMILES: O=C(NC1(CO)CCC1)OCCc1c(-c2ccc(F)cc2)[nH]c2c(F)cc(F)cc12] | 436 |
| 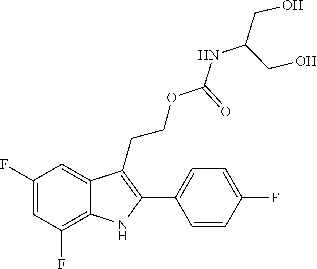 [SMILES: O=C(NC(CO)CO)OCCc1c(-c2ccc(F)cc2)[nH]c2c(F)cc(F)cc12] | 437 |
| 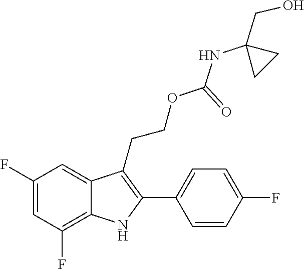 [SMILES: O=C(NC1(CO)CC1)OCCc1c(-c2ccc(F)cc2)[nH]c2c(F)cc(F)cc12] | 438 |
| 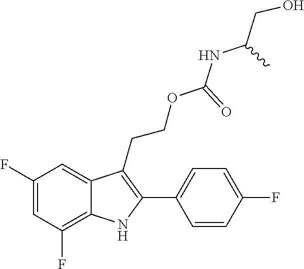 [SMILES: CC(CO)NC(=O)OCCc1c(-c2ccc(F)cc2)[nH]c2c(F)cc(F)cc12] | 439 |
| 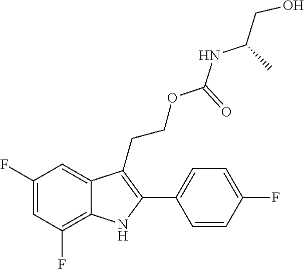 [SMILES: C[C@@H](CO)NC(=O)OCCc1c(-c2ccc(F)cc2)[nH]c2c(F)cc(F)cc12] | 440 |
| 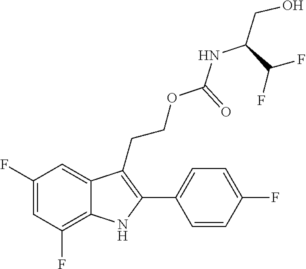 [SMILES: O=C(N[C@@H](CO)C(F)F)OCCc1c(-c2ccc(F)cc2)[nH]c2c(F)cc(F)cc12] | 441 |
| 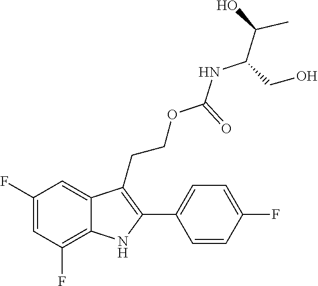 [SMILES: C[C@H](O)[C@H](CO)NC(=O)OCCc1c(-c2ccc(F)cc2)[nH]c2c(F)cc(F)cc12] | 442 |
| 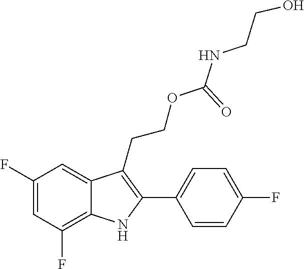 [SMILES: O=C(NCCO)OCCc1c(-c2ccc(F)cc2)[nH]c2c(F)cc(F)cc12] | 443 |
| 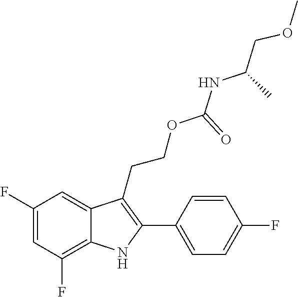 [SMILES: COC[C@H](C)NC(=O)OCCc1c(-c2ccc(F)cc2)[nH]c2c(F)cc(F)cc12] | 444 |
| 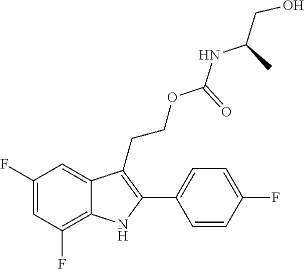 [SMILES: C[C@H](CO)NC(=O)OCCc1c(-c2ccc(F)cc2)[nH]c2c(F)cc(F)cc12] | 445 |
| 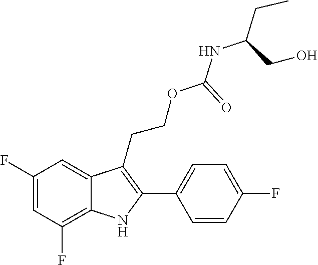 [SMILES: CC[C@@H](CO)NC(=O)OCCc1c(-c2ccc(F)cc2)[nH]c2c(F)cc(F)cc12] | 446 |
| 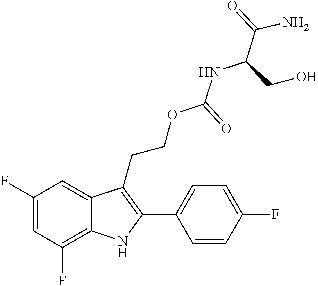 [SMILES: NC(=O)[C@@H](CO)NC(=O)OCCc1c(-c2ccc(F)cc2)[nH]c2c(F)cc(F)cc12] | 447 |
| 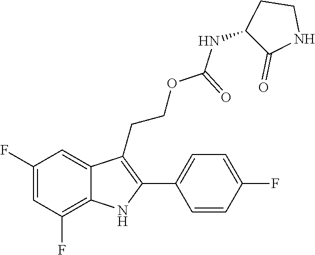 [SMILES: O=C(N[C@@H]1CCNC1=O)OCCc1c(-c2ccc(F)cc2)[nH]c2c(F)cc(F)cc12] | 448 |
| 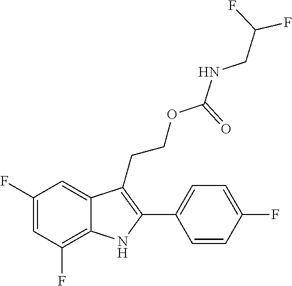 [SMILES: O=C(NCC(F)F)OCCc1c(-c2ccc(F)cc2)[nH]c2c(F)cc(F)cc12] | 449 |
| 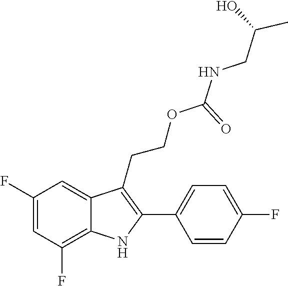 [SMILES: C[C@@H](O)CNC(=O)OCCc1c(-c2ccc(F)cc2)[nH]c2c(F)cc(F)cc12] | 450 |
| 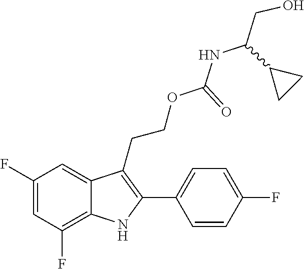 [SMILES: O=C(NC(CO)C1CC1)OCCc1c(-c2ccc(F)cc2)[nH]c2c(F)cc(F)cc12] | 451 |
| 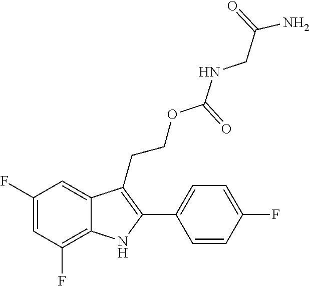 [SMILES: NC(=O)CNC(=O)OCCc1c(-c2ccc(F)cc2)[nH]c2c(F)cc(F)cc12] | 452 |
| 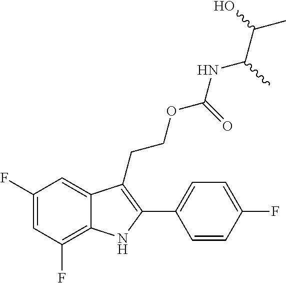 [SMILES: CC(O)C(C)NC(=O)OCCc1c(-c2ccc(F)cc2)[nH]c2c(F)cc(F)cc12] | 453 |
| 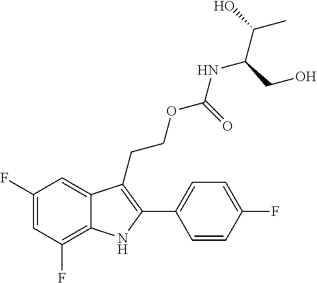 [SMILES: C[C@@H](O)[C@@H](CO)NC(=O)OCCc1c(-c2ccc(F)cc2)[nH]c2c(F)cc(F)cc12] | 454 |
| 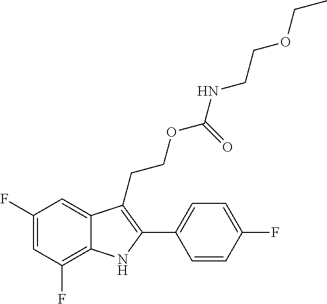 [SMILES: CCOCCNC(=O)OCCc1c(-c2ccc(F)cc2)[nH]c2c(F)cc(F)cc12] | 455 |
| 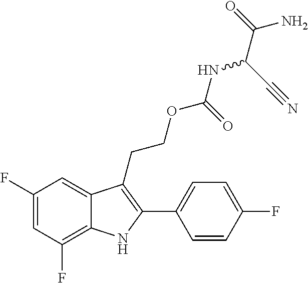 [SMILES: N#CC(NC(=O)OCCc1c(-c2ccc(F)cc2)[nH]c2c(F)cc(F)cc12)C(N)=O] | 456 |
| 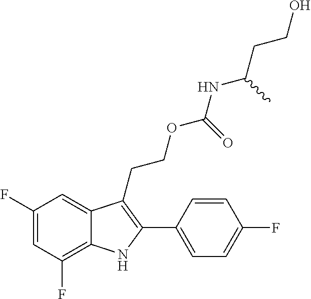 [SMILES: CC(CCO)NC(=O)OCCc1c(-c2ccc(F)cc2)[nH]c2c(F)cc(F)cc12] | 457 |
| 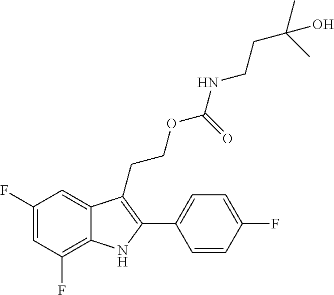 [SMILES: CC(C)(O)CCNC(=O)OCCc1c(-c2ccc(F)cc2)[nH]c2c(F)cc(F)cc12] | 458 |
| 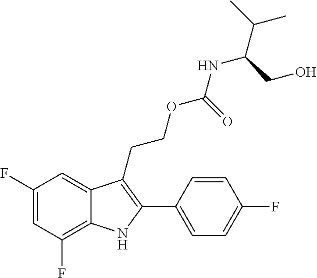 [SMILES: CC(C)[C@@H](CO)NC(=O)OCCc1c(-c2ccc(F)cc2)[nH]c2c(F)cc(F)cc12] | 459 |
| 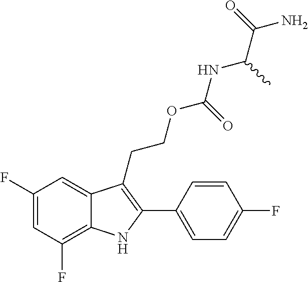 [SMILES: CC(NC(=O)OCCc1c(-c2ccc(F)cc2)[nH]c2c(F)cc(F)cc12)C(N)=O] | 460 |
| 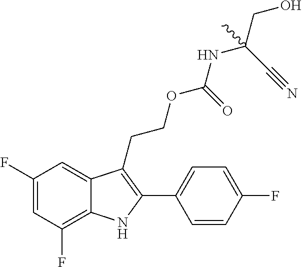 [SMILES: CC(C#N)(CO)NC(=O)OCCc1c(-c2ccc(F)cc2)[nH]c2c(F)cc(F)cc12] | 461 |
| 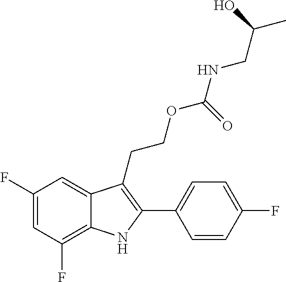 [SMILES: C[C@H](O)CNC(=O)OCCc1c(-c2ccc(F)cc2)[nH]c2c(F)cc(F)cc12] | 462 |
| 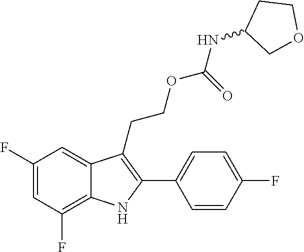 [SMILES: O=C(NC1CCOC1)OCCc1c(-c2ccc(F)cc2)[nH]c2c(F)cc(F)cc12] | 463 |
| 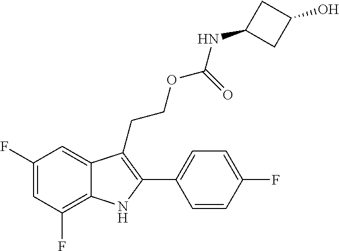 [SMILES: O=C(N[C@H]1C[C@H](O)C1)OCCc1c(-c2ccc(F)cc2)[nH]c2c(F)cc(F)cc12] | 464 |
| 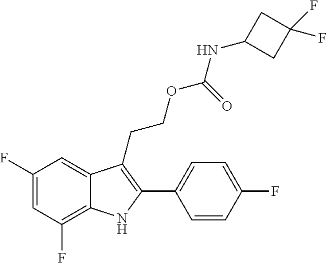 [SMILES: O=C(NC1CC(F)(F)C1)OCCc1c(-c2ccc(F)cc2)[nH]c2c(F)cc(F)cc12] | 465 |
| 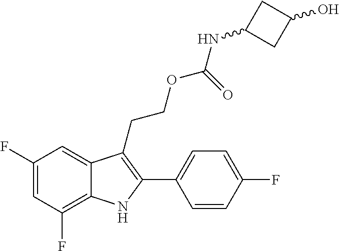 [SMILES: O=C(NC1CC(O)C1)OCCc1c(-c2ccc(F)cc2)[nH]c2c(F)cc(F)cc12] | 466 |
| 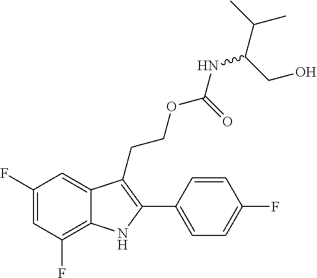 [SMILES: CC(C)C(CO)NC(=O)OCCc1c(-c2ccc(F)cc2)[nH]c2c(F)cc(F)cc12] | 467 |
| 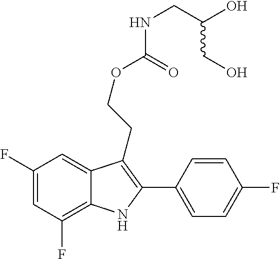 [SMILES: O=C(NCC(O)CO)OCCc1c(-c2ccc(F)cc2)[nH]c2c(F)cc(F)cc12] | 468 |
| 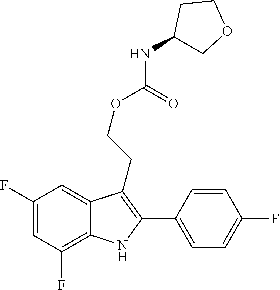 [SMILES: O=C(N[C@H]1CCOC1)OCCc1c(-c2ccc(F)cc2)[nH]c2c(F)cc(F)cc12] | 469 |
| 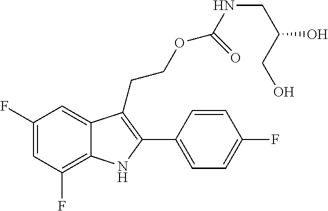 [SMILES: O=C(NC[C@H](O)CO)OCCc1c(-c2ccc(F)cc2)[nH]c2c(F)cc(F)cc12] | 470 |
| 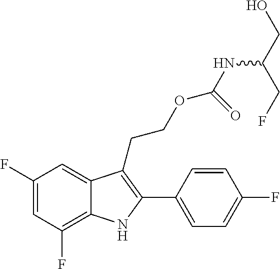 [SMILES: O=C(NC(CO)CF)OCCc1c(-c2ccc(F)cc2)[nH]c2c(F)cc(F)cc12] | 471 |
| 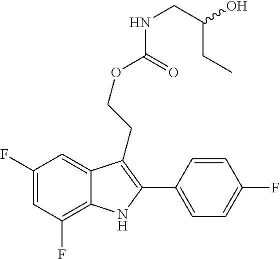 [SMILES: CCC(O)CNC(=O)OCCc1c(-c2ccc(F)cc2)[nH]c2c(F)cc(F)cc12] | 472 |
| 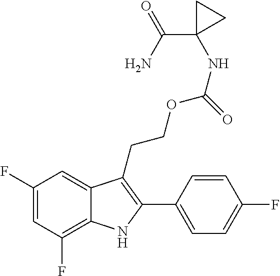 [SMILES: NC(=O)C1(NC(=O)OCCc2c(-c3ccc(F)cc3)[nH]c3c(F)cc(F)cc23)CC1] | 473 |
| 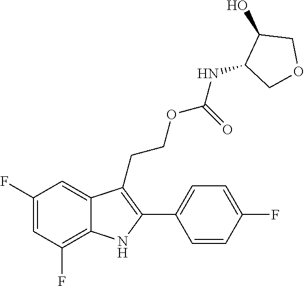 [SMILES: O=C(N[C@H]1COC[C@@H]1O)OCCc1c(-c2ccc(F)cc2)[nH]c2c(F)cc(F)cc12] | 474 |
| 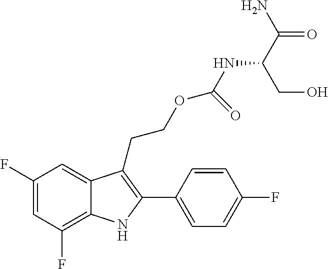 [SMILES: NC(=O)[C@H](CO)NC(=O)OCCc1c(-c2ccc(F)cc2)[nH]c2c(F)cc(F)cc12] | 475 |
| 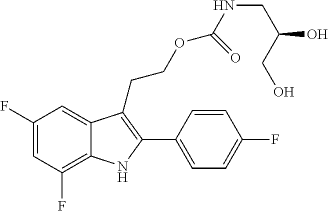 [SMILES: O=C(NC[C@@H](O)CO)OCCc1c(-c2ccc(F)cc2)[nH]c2c(F)cc(F)cc12] | 476 |
| 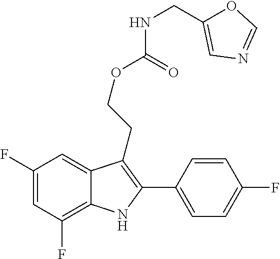 [SMILES: O=C(NCc1cnco1)OCCc1c(-c2ccc(F)cc2)[nH]c2c(F)cc(F)cc12] | 477 |
| 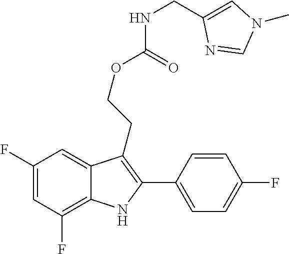 [SMILES: Cn1cnc(CNC(=O)OCCc2c(-c3ccc(F)cc3)[nH]c3c(F)cc(F)cc23)c1] | 478 |
| 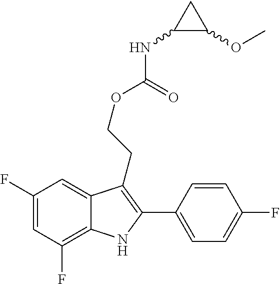 [SMILES: COC1CC1NC(=O)OCCc1c(-c2ccc(F)cc2)[nH]c2c(F)cc(F)cc12] | 479 |
| 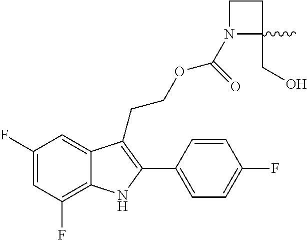 [SMILES: CC1(CO)CCN1C(=O)OCCc1c(-c2ccc(F)cc2)[nH]c2c(F)cc(F)cc12] | 480 |
| 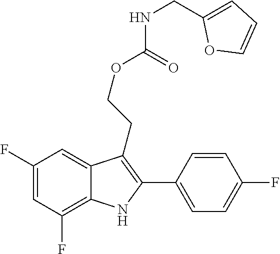 [SMILES: O=C(NCc1ccco1)OCCc1c(-c2ccc(F)cc2)[nH]c2c(F)cc(F)cc12] | 481 |
| 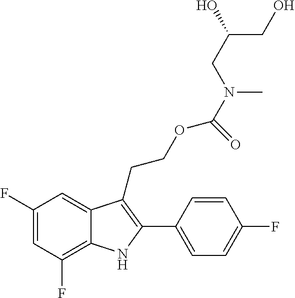 [SMILES: CN(C[C@H](O)CO)C(=O)OCCc1c(-c2ccc(F)cc2)[nH]c2c(F)cc(F)cc12] | 482 |
| 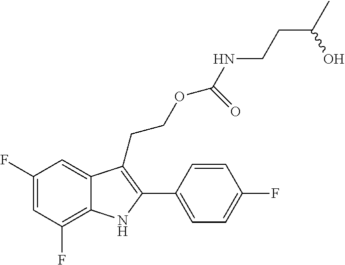 [SMILES: CC(O)CCNC(=O)OCCc1c(-c2ccc(F)cc2)[nH]c2c(F)cc(F)cc12] | 483 |
| 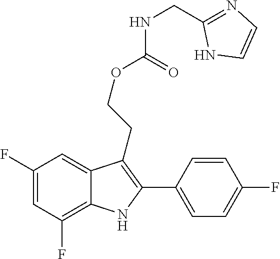 [SMILES: O=C(NCc1ncc[nH]1)OCCc1c(-c2ccc(F)cc2)[nH]c2c(F)cc(F)cc12] | 484 |
| 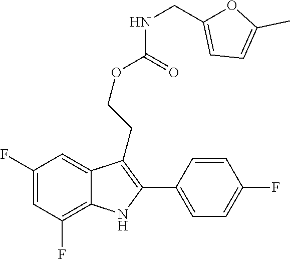 [SMILES: Cc1ccc(CNC(=O)OCCc2c(-c3ccc(F)cc3)[nH]c3c(F)cc(F)cc23)o1] | 485 |
| 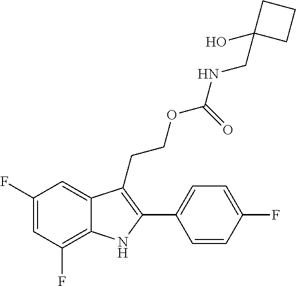 [SMILES: O=C(NCC1(O)CCC1)OCCc1c(-c2ccc(F)cc2)[nH]c2c(F)cc(F)cc12] | 486 |
| 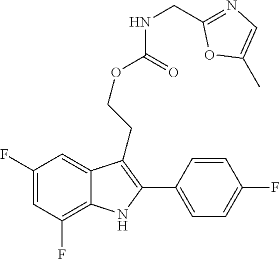 [SMILES: Cc1cnc(CNC(=O)OCCc2c(-c3ccc(F)cc3)[nH]c3c(F)cc(F)cc23)o1] | 487 |
| 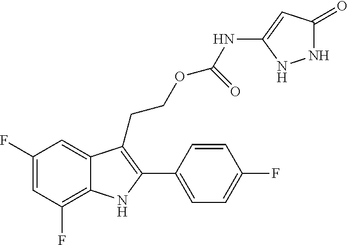 [SMILES: O=C(Nc1cc(=O)[nH][nH]1)OCCc1c(-c2ccc(F)cc2)[nH]c2c(F)cc(F)cc12] | 488 |
| 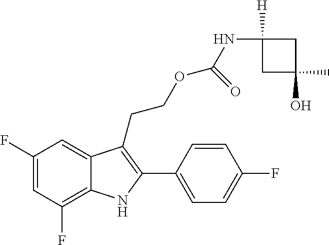 [SMILES: C[C@]1(O)C[C@@H](NC(=O)OCCc2c(-c3ccc(F)cc3)[nH]c3c(F)cc(F)cc23)C1] | 489 |
| 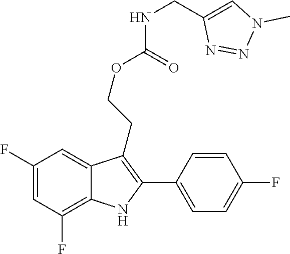 [SMILES: Cn1cc(CNC(=O)OCCc2c(-c3ccc(F)cc3)[nH]c3c(F)cc(F)cc23)nn1] | 490 |
| 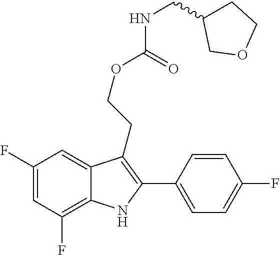 [SMILES: O=C(NCC1CCOC1)OCCc1c(-c2ccc(F)cc2)[nH]c2c(F)cc(F)cc12] | 491 |
| 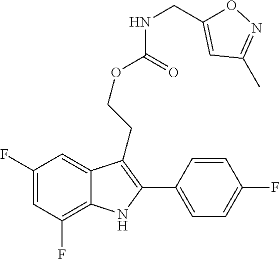 [SMILES: Cc1cc(CNC(=O)OCCc2c(-c3ccc(F)cc3)[nH]c3c(F)cc(F)cc23)on1] | 492 |
| 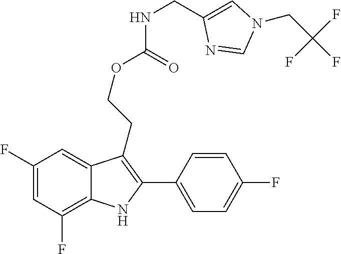 [SMILES: O=C(NCc1cn(CC(F)(F)F)cn1)OCCc1c(-c2ccc(F)cc2)[nH]c2c(F)cc(F)cc12] | 493 |
| 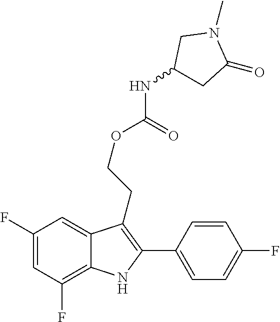 [SMILES: CN1CC(NC(=O)OCCc2c(-c3ccc(F)cc3)[nH]c3c(F)cc(F)cc23)CC1=O] | 494 |
| 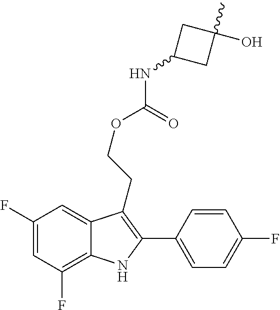 [SMILES: CC1(O)CC(NC(=O)OCCc2c(-c3ccc(F)cc3)[nH]c3c(F)cc(F)cc23)C1] | 495 |
| 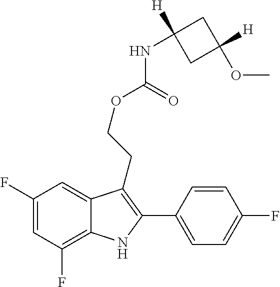 [SMILES: CO[C@H]1C[C@@H](NC(=O)OCCc2c(-c3ccc(F)cc3)[nH]c3c(F)cc(F)cc23)C1] | 496 |
| 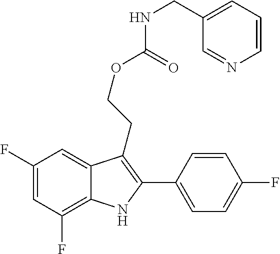 [SMILES: O=C(NCc1cccnc1)OCCc1c(-c2ccc(F)cc2)[nH]c2c(F)cc(F)cc12] | 497 |
| 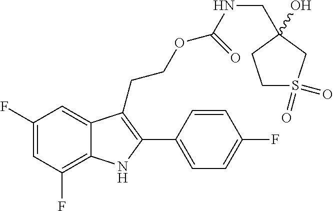 [SMILES: O=C(NCC1(O)CCS(=O)(=O)C1)OCCc1c(-c2ccc(F)cc2)[nH]c2c(F)cc(F)cc12] | 498 |
| 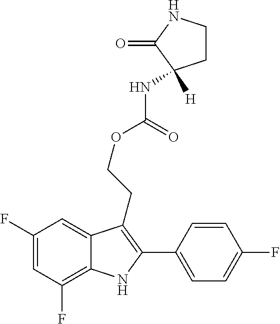 [SMILES: O=C(N[C@H]1CCNC1=O)OCCc1c(-c2ccc(F)cc2)[nH]c2c(F)cc(F)cc12] | 499 |
| 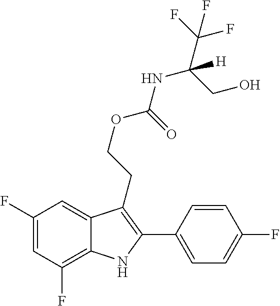 [SMILES: O=C(N[C@@H](CO)C(F)(F)F)OCCc1c(-c2ccc(F)cc2)[nH]c2c(F)cc(F)cc12] | 500 |
| 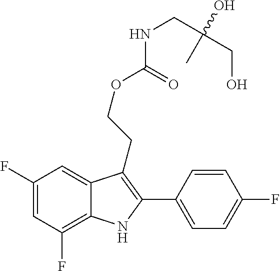 [SMILES: CC(O)(CO)CNC(=O)OCCc1c(-c2ccc(F)cc2)[nH]c2c(F)cc(F)cc12] | 501 |
| 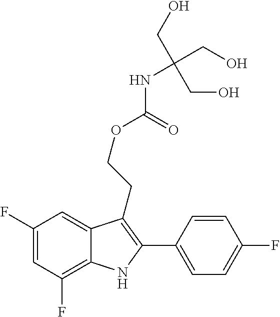 [SMILES: O=C(NC(CO)(CO)CO)OCCc1c(-c2ccc(F)cc2)[nH]c2c(F)cc(F)cc12] | 502 |
| 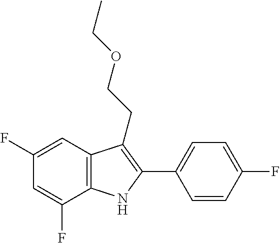 [SMILES: CCOCCc1c(-c2ccc(F)cc2)[nH]c2c(F)cc(F)cc12] | 503 |
| 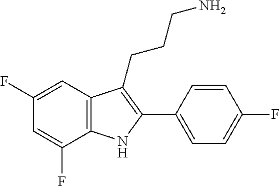 [SMILES: NCCCc1c(-c2ccc(F)cc2)[nH]c2c(F)cc(F)cc12] | 504 |
| 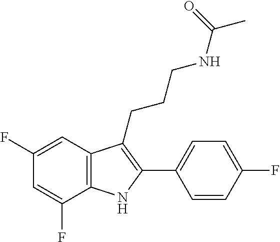 [SMILES: CC(=O)NCCCc1c(-c2ccc(F)cc2)[nH]c2c(F)cc(F)cc12] | 505 |
| 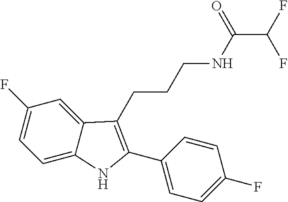 [SMILES: O=C(NCCCc1c(-c2ccc(F)cc2)[nH]c2ccc(F)cc12)C(F)F] | 506 |
| 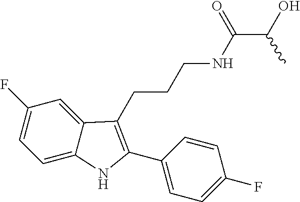 [SMILES: CC(O)C(=O)NCCCc1c(-c2ccc(F)cc2)[nH]c2ccc(F)cc12] | 507 |
| 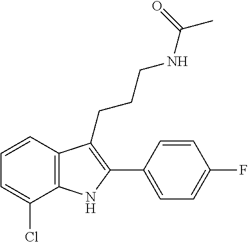 [SMILES: CC(=O)NCCCc1c(-c2ccc(F)cc2)[nH]c2c(Cl)cccc12] | 508 |
| 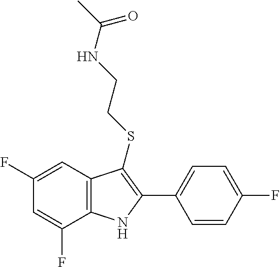 [SMILES: CC(=O)NCCSc1c(-c2ccc(F)cc2)[nH]c2c(F)cc(F)cc12] | 509 |
| 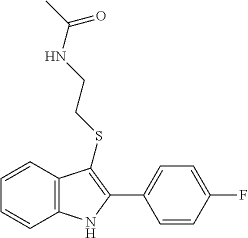 [SMILES: CC(=O)NCCSc1c(-c2ccc(F)cc2)[nH]c2ccccc12] | 510 |
| 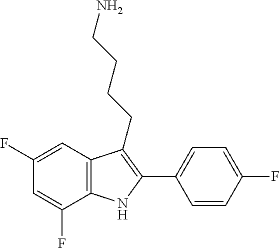 [SMILES: NCCCCc1c(-c2ccc(F)cc2)[nH]c2c(F)cc(F)cc12] | 511 |
| 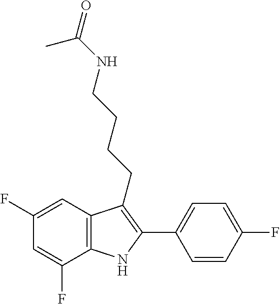 [SMILES: CC(=O)NCCCCc1c(-c2ccc(F)cc2)[nH]c2c(F)cc(F)cc12] | 512 |
| 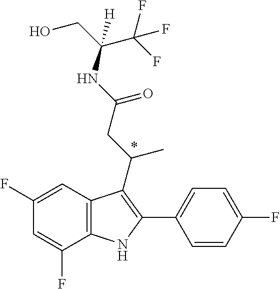 | 513 |
| 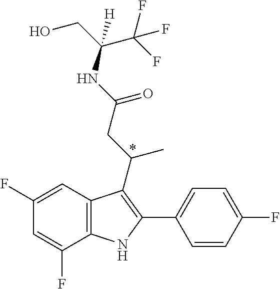 | 514 |
| 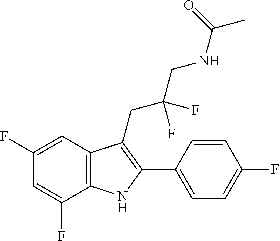 [SMILES: CC(=O)NCC(F)(F)Cc1c(-c2ccc(F)cc2)[nH]c2c(F)cc(F)cc12] | 515 |
| 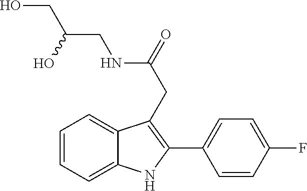 [SMILES: O=C(Cc1c(-c2ccc(F)cc2)[nH]c2ccccc12)NCC(O)CO] | 516 |
| 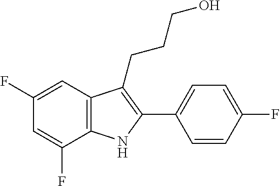 [SMILES: OCCCc1c(-c2ccc(F)cc2)[nH]c2c(F)cc(F)cc12] | 517 |
| 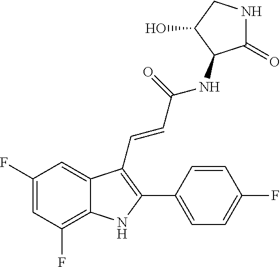 [SMILES: O=C(/C=C/c1c(-c2ccc(F)cc2)[nH]c2c(F)cc(F)cc12)N[C@@H]1C(=O)NC[C@H]1O] | 518 |
| 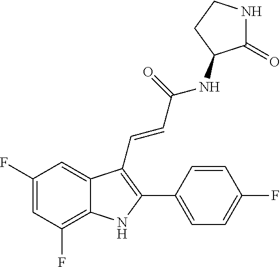 [SMILES: O=C(/C=C/c1c(-c2ccc(F)cc2)[nH]c2c(F)cc(F)cc12)N[C@H]1CCNC1=O] | 519 |
| 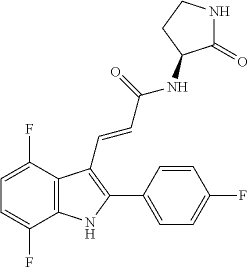 [SMILES: O=C(/C=C/c1c(-c2ccc(F)cc2)[nH]c2c(F)ccc(F)c12)N[C@H]1CCNC1=O] | 520 |
| 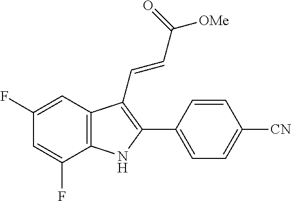 [SMILES: COC(=O)/C=C/c1c(-c2ccc(C#N)cc2)[nH]c2c(F)cc(F)cc12] | 521 |
| 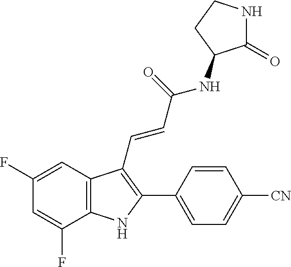 [SMILES: N#Cc1ccc(-c2[nH]c3c(F)cc(F)cc3c2/C=C/C(=O)N[C@H]2CCNC2=O)cc1] | 522 |
| 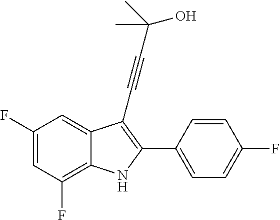 [SMILES: CC(C)(O)C#Cc1c(-c2ccc(F)cc2)[nH]c2c(F)cc(F)cc12] | 523 |
| 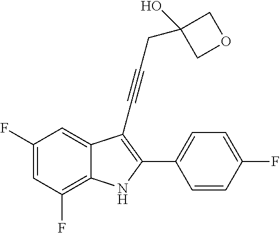 [SMILES: OC1(CC#Cc2c(-c3ccc(F)cc3)[nH]c3c(F)cc(F)cc23)COC1] | 524 |
| 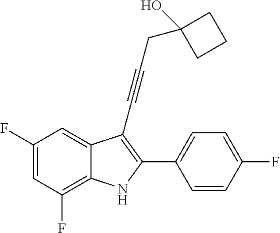 [SMILES: OC1(CC#Cc2c(-c3ccc(F)cc3)[nH]c3c(F)cc(F)cc23)CCC1] | 525 |
| 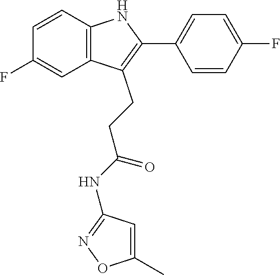 [SMILES: Cc1cc(NC(=O)CCc2c(-c3ccc(F)cc3)[nH]c3ccc(F)cc23)no1] | 526 |
| 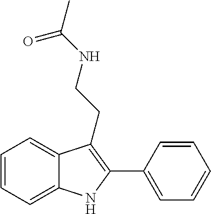 [SMILES: CC(=O)NCCc1c(-c2ccccc2)[nH]c2ccccc12] | 527 |

Another aspect of the disclosure provides methods for making compounds of Formula I, I′, II, or II′, Compounds 1 to 527, deuterated derivatives of those compounds, and pharmaceutically acceptable salts of any of foregoing. The disclosure also provides intermediates for making any of compounds, deuterated derivatives, or pharmaceutically acceptable salts disclosed herein.

Another aspect of the disclosure provides pharmaceutical compositions comprising at least one compound, deuterated derivative, or pharmaceutically acceptable salt chosen from compounds of Formula I, I′, II, or II′, Compounds 1 to 527, deuterated derivatives of those compounds, and pharmaceutically acceptable salts of any of foregoing. In some embodiments, the pharmaceutical composition comprising at least one compound, deuterated derivative, or pharmaceutically acceptable salt chosen from compounds of Formula I, I′, II, or II′, Compounds 1 to 527, deuterated derivatives of those compounds, and pharmaceutically acceptable salts of any of foregoing is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include at least one additional active therapeutic agent. Alternatively, a pharmaceutical composition comprising at least one compound, deuterated derivative, or pharmaceutically acceptable salt chosen from compounds of Formula I, I′, II, or II′, Compounds 1 to 527, deuterated derivatives of those compounds, and pharmaceutically acceptable salts of any of foregoing can be administered as a separate pharmaceutical composition concurrently with, prior to, or subsequent to, a composition comprising at least one other active therapeutic agent. In some embodiments, a pharmaceutical composition comprising at least one compound, deuterated derivative, or pharmaceutically acceptable salt chosen from compounds of Formula I, I′, II, or II′, Compounds 1 to 527, deuterated derivatives of those compounds, and pharmaceutically acceptable salts of any of foregoing can be administered as a separate pharmaceutical composition concurrently with, prior to, or subsequent to, a composition comprising at least one other active therapeutic agent.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as, e.g., human serum albumin), buffer substances (such as, e.g., phosphates, glycine, sorbic acid, and potassium sorbate), partial glyceride mixtures of saturated vegetable fatty acids, water, salts, and electrolytes (such as, e.g., protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as, e.g., lactose, glucose, and sucrose), starches (such as, e.g., corn starch and potato starch), cellulose and its derivatives (such as, e.g., sodium carboxymethyl cellulose, ethyl cellulose, and cellulose acetate), powdered tragacanth, malt, gelatin, talc, excipients (such as, e.g., cocoa butter and suppository waxes), oils (such as, e.g., peanut oil, cottonseed oil, safflower oil, sesame oil, olive oil, corn oil, and soybean oil), glycols (such as, e.g., propylene glycol and polyethylene glycol), esters (such as, e.g., ethyl oleate and ethyl laurate), agar, buffering agents (such as, e.g., magnesium hydroxide and aluminum hydroxide), alginic acid, pyrogen-free water, isotonic saline, Ringer's solution, ethyl alcohol, phosphate buffer solutions, non-toxic compatible lubricants (such as, e.g., sodium lauryl sulfate and magnesium stearate), coloring agents, releasing agents, coating agents, sweetening agents, flavoring agents, perfuming agents, preservatives, and antioxidants.

In some embodiments of the disclosure, the compounds and the pharmaceutical compositions described herein are used to treat APOL1 mediated kidney disease. In some embodiments, the APOL1 mediated kidney disease is chosen from ESKD, FSGS, HIV-associated nephropathy, NDKD, arterionephrosclerosis, lupus nephritis, microalbuminuria, and chronic kidney disease. In some embodiments, the APOL1 mediated kidney disease treated with the compound, deuterated derivative, pharmaceutically acceptable salt, and/or composition of the disclosure is FSGS. In some embodiments, the APOL1 mediated kidney disease treated with the compound, deuterated derivative, pharmaceutically acceptable salt, and/or composition of the disclosure is NDKD. In some embodiments, the APOL1 mediated kidney disease treated with the compound, deuterated derivative, and pharmaceutically acceptable salt and/or composition of the disclosure is ESKD. In some embodiments, the patient with APOL1 mediated kidney disease to be treated with the compound, deuterated derivative, pharmaceutically acceptable salt, and/or composition of the disclosure has two APOL1 risk alleles. In some embodiments, the patient with APOL1 mediated kidney disease is homozygous for APOL1 genetic risk alleles G1: S342G:I384M. In some embodiments, the patient with APOL1 mediated kidney disease is homozygous for APOL1 genetic risk alleles G2: N388del:Y389del. In some embodiments, the patient with APOL1 mediated kidney disease is heterozygous for APOL1 genetic risk alleles G1: S342G:I384M and G2: N388del:Y389del.

In some embodiments, the methods of the disclosure comprise administering to a patient in need thereof at least one compound, deuterated derivative, or pharmaceutically acceptable salt chosen from compounds of Formula I, I′, II, or II′, Compounds 1 to 527, deuterated derivatives of those compounds, and pharmaceutically acceptable salts of any of foregoing. In some embodiments, the compound, deuterated derivative, or pharmaceutically acceptable salt is chosen from Compounds 1 to 527, deuterated derivatives of those compounds, and pharmaceutically acceptable salts of any of foregoing. In some embodiments, said patient in need thereof possesses APOL1 genetic variants, i.e., G1: S342G:I384M and G2: N388del:Y389del.

Another aspect of the disclosure provides methods of inhibiting APOL1 activity comprising contacting said APOL1 with at least one compound, deuterated derivative, or pharmaceutically acceptable salt chosen from compounds of Formula I, I′, II, or II′, Compounds 1 to 527, deuterated derivatives of those compounds, and pharmaceutically acceptable salts of any of foregoing. In some embodiments, the methods of inhibiting APOL1 activity comprise contacting said APOL1 with at least one compound, deuterated derivative, or pharmaceutically acceptable salt chosen from Compounds 1 to 527, deuterated derivatives of those compounds, and pharmaceutically acceptable salts of any of foregoing.

### Non-Limiting Exemplary Embodiments 1

Without limitation, some example embodiments of this disclosure include:

1. A compound, deuterated derivative, or pharmaceutically acceptable salt selected from compounds of Formula I:

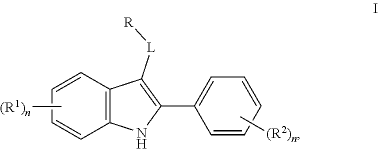

deuterated derivatives thereof, and pharmaceutically acceptable salts of any of the foregoing, wherein:
(i) R is selected from —C(O)NR³R⁴, —NR⁵C(O)R³, —NR⁵C(O)NR³R⁴, —NR³R⁴, —OR³, —NR⁵—SO₂R³, —OC(O)NR³R⁴, —C(O)OR³,

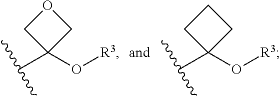

(ii) L is selected from divalent C₁-C₆ linear and branched alkyl (e.g., divalent C₁-C₆ linear and C₃-C₆ branched alkyl), divalent C₂-C₆ linear and branched alkenyl (e.g., divalent C₂-C₆ linear and C₃-C₆ branched alkenyl), divalent C₂-C₆ linear and branched alkynyl (e.g., divalent C₂-C₆ linear and C₃-C₆ branched alkynyl), and divalent 1- to 7-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;
   (iii) each R¹ is independently selected from:
halogen,
hydroxy,
thiol,
amino,
cyano,
C₁-C₆ linear, branched, and cyclic alkyl (e.g., C₁-C₆ linear, C₃-C₆ branched, and C₃-C₆ cyclic alkyl) optionally substituted with 1-3 groups independently selected from hydroxy and halogen,
C₂-C₆ linear, branched, and cyclic alkenyl (e.g., C₂-C₆ linear, C₃-C₆ branched, and C₃-C₆ cyclic alkenyl),
C₁-C₆ linear, branched, and cyclic alkoxy (e.g., C₁-C₆ linear, C₂-C₆ branched, and C₂-C₆ cyclic alkoxy) optionally substituted with 1-3 groups independently selected from halogen,
C₁-C₆ linear, branched, and cyclic thioalkyl (e.g., C₁-C₆ linear, C₂-C₆ branched, and C₂-C₆ cyclic thioalkyl) optionally substituted with 1-3 groups independently selected from halogen, and
C₁-C₆ linear, branched, and cyclic aminoalkyl optionally substituted with 1-3 groups independently selected from halogen,
or two IV groups, together with the carbon atoms to which they are attached, may form a C₄-C₈ cycloalkyl, aryl, or heteroaryl;
   (iv) each R² is independently selected from:
halogen,
hydroxy,
thiol,
amino,
cyano,
C₁-C₆ linear, branched, and cyclic alkyl (e.g., C₁-C₆ linear, C₃-C₆ branched, and C₃-C₆ cyclic alkyl) optionally substituted with 1-3 groups independently selected from hydroxy and halogen,
C₂-C₄ linear, branched, and cyclic alkenyl (e.g., C₂-C₄ linear, C₃-C₄ branched, and C₃-C₄ cyclic alkenyl),
C₁-C₆ linear, branched, and cyclic alkoxy (e.g., C₁-C₆ linear, C₂-C₆ branched, and C₂-C₆ cyclic alkoxy) optionally substituted with 1-3 groups independently selected from halogen,
C₁-C₄ linear, branched, and cyclic thioalkyl (e.g., C₁-C₄ linear, C₂-C₄ branched, and C₂-C₄ cyclic thioalkyl) optionally substituted with 1-3 groups independently selected from halogen, and
C₁-C₄ linear, branched, and cyclic aminoalkyl optionally substituted with 1-3 groups independently selected from halogen,
   (v) each n is independently selected from 0, 1, 2, 3, and 4;
   (vi) R³ and R⁴ are independently selected from:
hydrogen,
C₁-C₆ linear and branched alkylsulfonyl (e.g., C₁-C₆ linear and C₂-C₆ branched alkyl sulfonyl),
C₂-C₆ linear and branched alkenyl (e.g., C₂-C₆ linear and C₃-C₆ branched alkenyl),
amino optionally substituted with 1-2 groups independently selected from C₁-C₆ linear and branched alkyl (e.g., C₁-C₆ linear and C₃-C₆ branched alkyl) and C₃-C₆ cycloalkyl,
amide optionally substituted with 1-2 groups independently selected from C₁-C₃ alkyl,
C₁-C₆ linear and branched alkoxy (e.g., C₁-C₆ linear and C₂-C₆ branched alkoxy) optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl (e.g., C₃-C₆ cyclic alkyl) optionally substituted with 1-2 groups independently selected from:
   halogen,
   hydroxy,
   oxo,
   amino optionally substituted with 1-2 groups independently selected from hydrogen and C₁-C₆ linear or branched alkyl (e.g., C₁-C₆ linear or C₃-C₆ branched alkyl),
   aryl optionally substituted with 1-2 groups independently selected from halogen,
   C₁-C₆ linear and branched alkyl groups (e.g., C₁-C₆ linear and C₃-C₆ branched alkyl groups) (which may be further substituted with 1-3 groups independently selected from hydroxy, oxo, halogen, and C₁-C₆ linear and branched alkoxy groups (e.g., C₁-C₆ linear and C₂-C₆ branched alkoxy groups)),
   carbamate optionally substituted with 1-2 groups independently selected from C₁-C₆ linear and branched alkyl (e.g., C₁-C₆ linear and C₃-C₆ branched alkyl),
   C₁-C₆ linear and branched alkoxy (e.g., C₁-C₆ linear and C₂-C₆ branched alkoxy), and
   amide,
4- to 10-membered heterocyclyl optionally substituted with 1-3 groups independently selected from:
   halogen,
   oxo,
   hydroxy,
   amino, and
   C₁-C₆ linear and branched alkyl (e.g., C₁-C₆ linear and C₃-C₆ branched alkyl) (which may be further substituted with 1-2 groups independently selected from hydroxy, oxo, and C₁-C₆ linear and branched alkoxy (e.g., C₁-C₆ linear and C₂-C₆ branched alkoxy)),
aryl optionally substituted with 1-3 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy groups (e.g., C₁-C₆ linear and C₂-C₆ branched alkoxy groups)),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino), and
C₁-C₆ linear and branched alkyl groups (e.g., C₁-C₆ linear and C₃-C₆ branched alkyl groups), wherein the alkyl groups are optionally substituted with 1-4 groups independently selected from:
   amino groups optionally substituted with 1-2 groups independently selected from hydroxy, C₁-C₆ linear, branched, and cyclic alkyl (e.g., C₁-C₆ linear, C₃-C₆ branched, and C₃-C₆ cyclic alkyl) (which may be further substituted with 1-2 oxo), and C₁-C₆ linear and branched alkylsulfonyl (e.g., C₁-C₆ linear and C₂-C₆ branched alkylsulfonyl),
   hydroxy,
   oxo,
   cyano,
   carboxylic acid,
   sulfonic acid,
   —O-heteroaryl,
   carbamate optionally substituted with 1-2 groups independently selected from C₁-C₆ linear and branched alkyl (e.g., C₁-C₆ linear and C₃-C₆ branched alkyl),
   halogen,
   amido optionally substituted with 1-2 groups independently selected from hydroxy, C₁-C₆ linear, branched, and cyclic alkyl groups (e.g., C₁-C₆ linear, C₃-C₆ branched, and C₃-C₆ cyclic alkyl groups), and C₁-C₆ linear, branched, and cyclic hydroxyalkyl (e.g., C₁-C₆ linear, C₃-C₆ branched, and C₃-C₆ cyclic hydroxyalkyl groups),
   C₃-C₆ cyclic alkyl optionally substituted with 1-2 groups independently selected from halogen, hydroxy, C₁-C₆ linear and branched hydroxyalkyl (e.g., C₁-C₆ linear and C₃-C₆ branched hydroxyalkyl), C₁-C₆ linear and branched alkoxy (e.g., C₁-C₆ linear and C₂-C₆ branched alkoxy), and carbamate (which may be further substituted with 1-2 groups independently selected from C₁-C₆ linear and branched alkyl (e.g., C₁-C₆ linear and C₃-C₆ branched alkyl)),
   C₁-C₆ linear and branched alkynyl (e.g., C₂-C₆ linear and branched alkynyl, e.g., C₂-C₆ linear and C₃-C₆ branched alkynyl),
   C₁-C₆ linear and branched alkoxy (e.g., C₁-C₆ linear and C₂-C₆ branched alkoxy) optionally substituted with 1-2 hydroxy,
   C₁-C₆ linear and branched alkylsulfonyl (e.g., C₁-C₆ linear and C₂-C₆ branched alkylsulfonyl),
   aryl optionally substituted with 1-2 groups independently selected from halogen groups, hydroxy, and C₁-C₆ linear and branched alkyl groups (e.g., C₁-C₆ linear and C₃-C₆ branched alkyl groups) (which may be further substituted with 1-2 groups independently selected from hydroxy and C₁-C₆ linear and branched alkoxy groups (e.g., C₁-C₆ linear and C₂-C₆ branched alkoxy groups)),
   carbonyl-(4-methylpiperazin-1-yl),
   carbonyl-(N-morpholino),
   4- to 10-membered heterocyclyl optionally substituted with 1-3 groups independently selected from halogen, oxo, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy (e.g., C₁-C₆ linear and C₂-C₆ branched alkoxy)), and
   4- to 10-membered heteroaryl optionally substituted with 1-3 groups independently selected from halogen, oxo, hydroxy, and C₁-C₆ linear, branched, and cyclic alkyl (e.g., C₁-C₆ linear, C₃-C₆ branched, and C₃-C₆ cyclic alkyl) (which may be further substituted with 1-3 groups independently selected from halogen, hydroxy, and C₁-C₆ linear and branched alkoxy (e.g., C₁-C₆ linear and C₂-C₆ branched alkoxy)),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl (e.g., C₁-C₆ linear, C₃-C₆ branched, and C₃-C₆ cyclic alkyl groups),
halogen,
hydroxy,
oxo,
C₁-C₆ linear, branched, and cyclic alkyl (e.g., C₁-C₆ linear, C₃-C₆ branched, and C₃-C₆ cyclic alkyl groups) optionally substituted with 1-2 groups independently selected from hydroxy, amino, C₁-C₆ linear, branched, and cyclic alkoxy groups (e.g., C₁-C₆ linear, C₂-C₆ branched, and C₂-C₆ cyclic alkoxy groups), and carbamate (which may be further substituted with 1-2 groups independently selected from C₁-C₆ linear and branched alkyl (e.g., C₁-C₆ linear and C₃-C₆ branched alkyl)),
amide optionally substituted with 1-2 groups independently selected from C₁-C₆ linear and branched alkyl (e.g., C₁-C₆ linear and C₃-C₆ branched alkyl),
carbamate optionally substituted with 1-2 groups independently selected from C₁-C₆ linear and branched alkyl (e.g., C₁-C₆ linear and C₃-C₆ branched alkyl),
carboxamide optionally substituted with 1-2 groups independently selected from C₁-C₆ linear and branched alkyl (e.g., C₁-C₆ linear and C₃-C₆ branched alkyl),
C₁-C₆ linear and branched alkoxy groups (e.g., C₁-C₆ linear and C₂-C₆ branched alkoxy groups) optionally substituted with 1-2 groups independently selected from C₁-C₆ linear, branched, and cyclic alkyl (e.g., C₁-C₆ linear, C₃-C₆ branched, and C₃-C₆ cyclic alkyl) and heterocyclyl,
4- to 10-membered heterocyclyl optionally substituted with 1-2 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy (e.g., C₁-C₆ linear and C₂-C₆ branched alkoxy)), and
4- to 10-membered heteroaryl optionally substituted with 1-2 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy groups (e.g., C₁-C₆ linear and C₂-C₆ branched alkoxy groups));

and

(vii) R⁵ is selected from hydrogen and C₁-C₆ linear or branched alkyl (e.g., C₁-C₆ linear or C₃-C₆ branched alkyl);
with the provisos that (1) the compound is not selected from

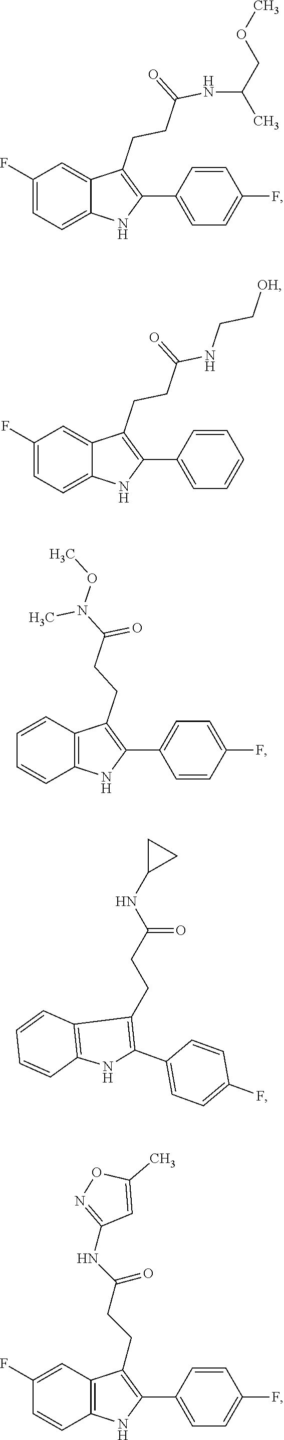 [SMILES: COCC(C)NC(=O)CCc1c(-c2ccc(F)cc2)[nH]c2ccc(F)cc12 ; O=C(CCc1c(-c2ccccc2)[nH]c2ccc(F)cc12)NCCO ; CON(C)C(=O)CCc1c(-c2ccc(F)cc2)[nH]c2ccccc12 ; O=C(CCc1c(-c2ccc(F)cc2)[nH]c2ccccc12)NC1CC1 ; Cc1cc(NC(=O)CCc2c(-c3ccc(F)cc3)[nH]c3ccc(F)cc23)no1]

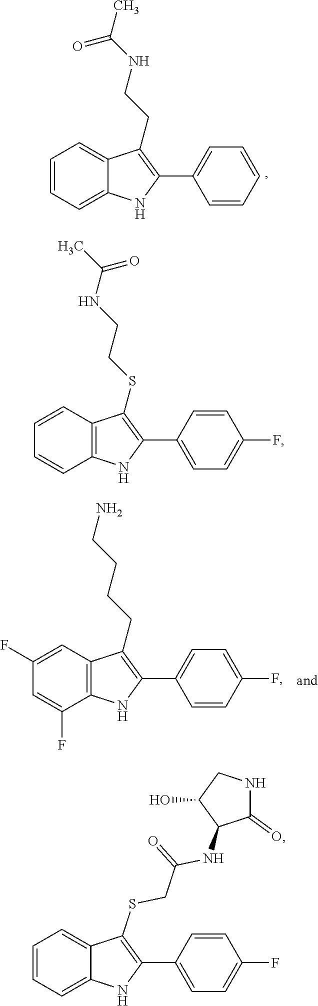

and

(2) when -L-R in Formula I is

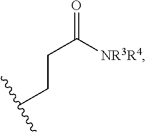

then R³ and R⁴ are not

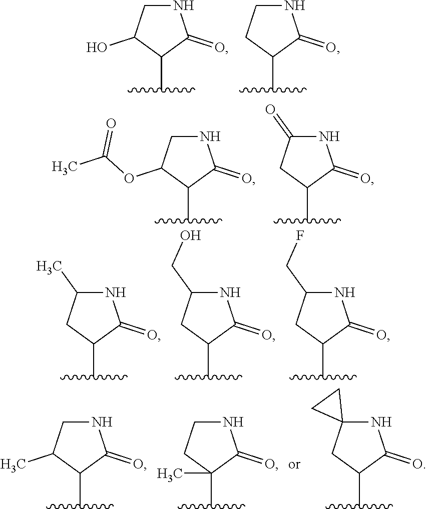

2. The compound, deuterated derivative, or pharmaceutically acceptable salt according to Embodiment 1, wherein L is selected from divalent C₁-C₆ linear and branched alkyl, divalent C₂-C₆ linear and branched alkenyl, divalent C₂-C₆ linear and branched alkynyl, and divalent 1- to 6-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino.
   3. The compound, deuterated derivative, or pharmaceutically acceptable salt according to Embodiment 1 or 2, wherein each R² is independently selected from:
halogen,
hydroxy,
thiol,
amino,
cyano,
C₁-C₄ linear, branched, and cyclic alkyl optionally substituted with 1-3 groups independently selected from hydroxy and halogen,
C₂-C₄ linear, branched, and cyclic alkenyl,
C₁-C₄ linear, branched, and cyclic alkoxy optionally substituted with 1-3 groups independently selected from halogen,
C₁-C₄ linear, branched, and cyclic thioalkyl optionally substituted with 1-3 groups independently selected from halogen, and
C₁-C₄ linear, branched, and cyclic aminoalkyl optionally substituted with 1-3 groups independently selected from halogen.
   4. The compound, deuterated derivative, or pharmaceutically acceptable salt according to any one of Embodiments 1-3, wherein R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl and C₃-C₆ cycloalkyl,
amide optionally substituted with 1-2 groups independently selected from C₁-C₃ alkyl, and
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl optionally substituted with 1-2 groups independently selected from:
   halogen,
   hydroxy,
   oxo,
   amino optionally substituted with 1-2 groups independently selected from hydrogen and C₁-C₆ linear or branched alkyl,
   aryl optionally substituted with 1-2 groups independently selected from halogen,
   C₁-C₆ linear and branched alkyl groups (which may be further substituted with 1-3 groups independently selected from hydroxy, oxo, halogen, and C₁-C₆ linear and branched alkoxy groups),
   carbamate optionally substituted with 1-2 groups independently selected from C₁-C₆ linear and branched alkyl,
   C₁-C₆ linear and branched alkoxy, and
   amide,
4- to 10-membered heterocyclyl optionally substituted with 1-3 groups independently selected from:
   halogen,
   oxo,
   hydroxy, and
   C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, and C₁-C₆ linear and branched alkyl (which may be further substituted with one or two groups independently selected from hydroxy and C₁-C₆ linear and branched alkoxy groups),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino),
C₁-C₆ linear and branched alkyl groups, wherein the alkyl groups are optionally substituted with 1-4 groups independently selected from:
   amino groups optionally substituted with 1-2 groups independently selected from C₁-C₆ linear, branched, and cyclic alkyl (which may be further substituted with 1-2 oxo), and C₁-C₆ linear and branched alkylsulfonyl,
   hydroxy,
   oxo,
   cyano,
   carboxylic acid,
   sulfonic acid,
   —O-heteroaryl,
   carbamate optionally substituted with 1-2 groups independently selected from C₁-C₆ linear and branched alkyl,
   halogen,
   amido optionally substituted with 1-2 groups independently selected from hydroxy, C₁-C₆ linear, branched, and cyclic alkyl groups and C₁-C₆ linear, branched, and cyclic hydroxyalkyl,
   C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
   C₁-C₆ linear and branched alkynyl,
   C₁-C₆ linear and branched alkoxy optionally substituted with 1-2 hydroxy,
   C₁-C₆ linear and branched alkylsulfonyl,
   aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
   carbonyl-(4-methylpiperazin-1-yl),
   carbonyl-(N-morpholino),
   4- to 10-membered heterocyclyl optionally substituted with 1-2 groups independently selected from halogen, oxo, hydroxy, and C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-3 groups independently selected from halogen, oxo, hydroxy, and C₁-C₆ linear, branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl,
halogen,
hydroxy,
oxo,
C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
amide optionally substituted with 1-2 groups independently selected from C₁-C₆ linear and branched alkyl,
carbamate optionally substituted with 1-2 groups independently selected from C₁-C₆ linear and branched alkyl,
carboxamide optionally substituted with 1-2 groups independently selected from C₁-C₆ linear and branched alkyl,
C₁-C₆ linear and branched alkoxy groups optionally substituted with 1-2 groups independently selected from C₁-C₆ linear, branched, and cyclic alkyl, and heterocyclyl,
4- to 10-membered heterocyclyl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups).
   5. The compound, deuterated derivative, or pharmaceutically acceptable salt according to Embodiment 1, wherein:

L is selected from divalent C₁-C₆ linear and branched alkyl, divalent C₂-C₆ linear and branched alkenyl, divalent C₂-C₆ linear and branched alkynyl, and divalent 1- to 6-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;

each R² is independently selected from:
halogen,
hydroxy,
thiol,
amino,
cyano,
C₁-C₄ linear, branched, and cyclic alkyl optionally substituted with 1-3 groups independently selected from hydroxy and halogen,
C₂-C₄ linear, branched, and cyclic alkenyl,
C₁-C₄ linear, branched, and cyclic alkoxy optionally substituted with 1-3 groups independently selected from halogen,
C₁-C₄ linear, branched, and cyclic thioalkyl optionally substituted with 1-3 groups independently selected from halogen, and
C₁-C₄ linear, branched, and cyclic aminoalkyl optionally substituted with 1-3 groups independently selected from halogen; and

R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl and C₃-C₆ cycloalkyl,
amide optionally substituted with 1-2 groups independently selected from C₁-C₃ alkyl, and
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl optionally substituted with 1-2 groups independently selected from:
   halogen,
   hydroxy,
   oxo,
   amino optionally substituted with 1-2 groups independently selected from hydrogen and C₁-C₆ linear or branched alkyl,
   aryl optionally substituted with 1-2 groups independently selected from halogen,
   C₁-C₆ linear and branched alkyl groups (which may be further substituted with 1-3 groups independently selected from hydroxy, oxo, halogen, and C₁-C₆ linear and branched alkoxy groups),
   carbamate optionally substituted with 1-2 groups independently selected from C₁-C₆ linear and branched alkyl,
   C₁-C₆ linear and branched alkoxy, and
   amide,
4- to 10-membered heterocyclyl optionally substituted with 1-3 groups independently selected from:
   halogen,
   oxo,
   hydroxy, and
   C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, and C₁-C₆ linear and branched alkyl (which may be further substituted with one or two groups independently selected from hydroxy and C₁-C₆ linear and branched alkoxy groups),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino),
C₁-C₆ linear and branched alkyl groups, wherein the alkyl groups are optionally substituted with 1-4 groups independently selected from:
   amino groups optionally substituted with 1-2 groups independently selected from C₁-C₆ linear, branched, and cyclic alkyl (which may be further substituted with 1-2 oxo), and C₁-C₆ linear and branched alkylsulfonyl,
   hydroxy,
   oxo,
   cyano,
   carboxylic acid,
   sulfonic acid,
   —O-heteroaryl,
   carbamate optionally substituted with 1-2 groups independently selected from C₁-C₆ linear and branched alkyl,
   halogen,
   amido optionally substituted with 1-2 groups independently selected from hydroxy, C₁-C₆ linear, branched, and cyclic alkyl groups and C₁-C₆ linear, branched, and cyclic hydroxyalkyl,
   C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
   C₁-C₆ linear and branched alkynyl,
   C₁-C₆ linear and branched alkoxy optionally substituted with 1-2 hydroxy,
   C₁-C₆ linear and branched alkylsulfonyl,
   aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
   carbonyl-(4-methylpiperazin-1-yl),
   carbonyl-(N-morpholino),
   4- to 10-membered heterocyclyl optionally substituted with 1-2 groups independently selected from halogen, oxo, hydroxy, and C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-3 groups independently selected from halogen, oxo, hydroxy, and C₁-C₆ linear, branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl,
halogen,
hydroxy,
oxo,
C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
amide optionally substituted with 1-2 groups independently selected from C₁-C₆ linear and branched alkyl,
carbamate optionally substituted with 1-2 groups independently selected from C₁-C₆ linear and branched alkyl,
carboxamide optionally substituted with 1-2 groups independently selected from C₁-C₆ linear and branched alkyl,
C₁-C₆ linear and branched alkoxy groups optionally substituted with 1-2 groups independently selected from C₁-C₆ linear, branched, and cyclic alkyl, and heterocyclyl,
4- to 10-membered heterocyclyl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups).
   6. The compound, deuterated derivative, or pharmaceutically acceptable salt according to any one of Embodiments 1-5, wherein, when L is a divalent C₂ linear alkyl optionally substituted with 1-2 groups independently selected from methyl, halogen, and hydroxy and R is —NR³R⁴, then R³ and R⁴ are not

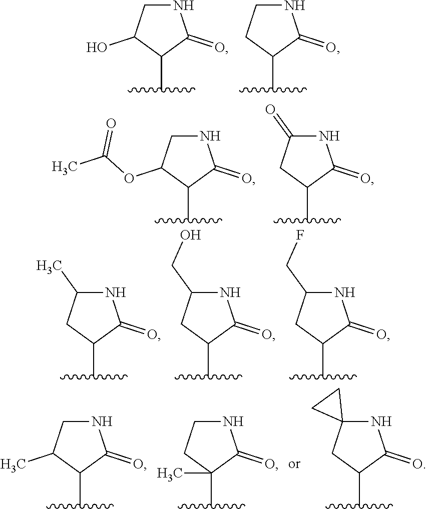

7. The compound, deuterated derivative, or pharmaceutically acceptable salt according to any one of Embodiments 1-6, wherein, when L is a divalent C₂ linear alkyl optionally substituted with 1-2 groups independently selected from methyl, halogen, and hydroxy and R is —NR³R⁴, then R³ and R⁴ are not

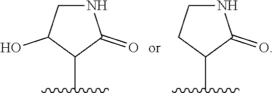

8. The compound, deuterated derivative, or pharmaceutically acceptable salt according to any one of Embodiments 1-7, wherein each R¹ is independently selected from halogen, hydroxy, amino, C₁-C₆ linear and branched alkyl (optionally substituted with 1-3 groups independently selected from hydroxy and halogen), C₃-C₆ cycloalkyl, and C₁-C₆ linear and branched alkoxy (optionally substituted with 1-3 groups independently selected from halogen).
9. The compound, deuterated derivative, or pharmaceutically acceptable salt according to any one of Embodiments 1, 2, 4, 6, and 7, wherein each R² is independently selected from halogen, hydroxy, amino, cyano, C₁-C₆ linear and branched alkyl (optionally substituted with 1-3 groups independently selected from hydroxy and halogen), and C₁-C₆ linear and branched alkoxy (optionally substituted with 1-3 groups independently selected from halogen).
10. The compound, deuterated derivative, or pharmaceutically acceptable salt according to Embodiment 5, wherein each R² is independently selected from halogen, hydroxy, amino, cyano, C₁-C₄ linear and branched alkyl (optionally substituted with 1-3 groups independently selected from hydroxy and halogen), and C₁-C₄ linear and branched alkoxy (optionally substituted with 1-3 groups independently selected from halogen).
11. The compound, deuterated derivative, or pharmaceutically acceptable salt according to any one of Embodiments 1-10, wherein each R¹ and/or R² is fluorine.
12. The compound, deuterated derivative, or pharmaceutically acceptable salt according to any one of Embodiments 1-11, wherein each n is independently selected from 0, 1, and 2.
13. The compound, deuterated derivative, or pharmaceutically acceptable salt according to any one of Embodiments 1, 3, 4, and 6-12, wherein L is selected from divalent C₁-C₆ linear and branched alkyl, and divalent C₁-C₆ linear and branched thioalkyl, wherein the divalent alkyl and divalent thioalkyl are optionally substituted with 1-2 groups independently selected from halogen.
14. The compound, deuterated derivative, or pharmaceutically acceptable salt according to Embodiment 13, wherein L is selected from divalent C₁-C₃ linear and branched alkyl, and divalent C₁-C₃ linear and branched thioalkyl, wherein the divalent alkyl and divalent thioalkyl are optionally substituted with 1-2 groups independently selected from halogen.
15. The compound, deuterated derivative, or pharmaceutically acceptable salt according to Embodiment 5, wherein L is selected from divalent C₁-C₆ linear and branched alkyl and divalent C₁-C₅ linear and branched thioalkyl, wherein the divalent alkyl and divalent thioalkyl are optionally substituted with 1-2 groups independently selected from halogen.
16. The compound, deuterated derivative, or pharmaceutically acceptable salt according to any one of Embodiments 1-5 and 8-15, wherein R is —C(O)NR³R⁴, and wherein R³ and R⁴ are independently selected from:
hydrogen,
C₁-C₆ linear and branched alkylsulfonyl,
C₁-C₆ linear and branched alkoxy optionally substituted with 1-2 groups independently selected from hydroxy and oxo;
C₃-C₆ cyclic alkyl optionally substituted with 1-2 groups independently selected from:
   halogen,
   hydroxy,
   oxo,
   amino,
   aryl optionally substituted with 1-2 groups independently selected from halogen,
   C₁-C₆ linear and branched alkyl groups (which may be further substituted with 1-3 groups independently selected from hydroxy and halogen),
   carbamate optionally substituted with 1-2 groups independently selected from C₁-C₆ linear and branched alkyl,
   C₁-C₆ linear and branched alkoxy, and
   amido groups,
4- to 10-membered heterocyclyl optionally substituted with 1-3 groups independently selected from:
   oxo,
   hydroxy,
   C₁-C₆ linear and branched alkyl (which may be further substituted with 1-2 groups independently selected from hydroxy, oxo, and C₁-C₆ linear and branched alkoxy),
4- to 10-membered heteroaryl optionally substituted with 1-3 groups independently selected from:
   hydroxy,
   oxo,
   halogen, and
   C₁-C₆ linear alkyl (which may be further substituted with 1-3 groups independently selected from halogen),
C₁-C₆ linear and branched alkyl groups, wherein the alkyl groups are optionally substituted with 1-4 groups independently selected from:
   amino groups optionally substituted with 1-2 groups independently selected from C₁-C₆ linear, branched, and cyclic alkyl,
   hydroxy,
   oxo,
   cyano,
   carbamate optionally substituted with 1-2 groups independently selected from C₁-C₆ linear and branched alkyl,
   halogen,
   amido,
   C₃-C₆ cyclic alkyl optionally substituted with 1-2 hydroxy,
   4- to 10-membered heterocyclyl optionally substituted with 1-2 groups independently selected from oxo, hydroxy, and C₁-C₆ linear and branched alkyl (which may be further substituted with 1-2 hydroxy), and
   4- to 10-membered heteroaryl optionally substituted with 1-3 groups independently selected from halogen and C₁-C₆ linear, branched, and cyclic alkyl (which may be further substituted with 1-3 groups independently selected from halogen),
or R³ and R⁴, together with the nitrogen atom to which they are attached, form a 4- to 10-membered heterocyclyl or heteroaryl optionally substituted with 1-3 groups independently selected from:
amino,
halogen,
hydroxy,
C₁-C₆ linear, branched, and cyclic alkyl optionally substituted with 1-2 groups independently selected from hydroxy, amino, and carbamate (which may be further substituted with 1-2 groups independently selected from C₁-C₆ linear and branched alkyl), and
carbamate optionally substituted with 1-2 groups independently selected from C₁-C₆ linear and branched alkyl.
   17. The compound, deuterated derivative, or pharmaceutically acceptable salt according to any one of Embodiments 1-3 and 8-15, wherein R is —NR⁵—C(O)R³, and wherein R³ is selected from:
hydrogen,
C₁-C₆ linear and branched alkyl optionally substituted with 1-4 groups independently selected from halogen, hydroxy, cyano, amido (which may be further substituted by 1-2 groups independently selected from C₁-C₃ alkyl), amino (which may be further substituted with C₁-C₃ alkylsulfonyl), carbamate (which may be further substituted with C₁-C₆ linear and branched alkyl), 4- to 6-membered heterocyclyl (which may be further substituted with 1-2 groups independently selected from halogen, oxo, and hydroxy), 4- to 6-membered heteroaryl (which may be further substituted with 1-2 groups independently selected from halogen, oxo, hydroxy, and C₁-C₃ alkyl), and C₃-C₆ cycloalkyl (which may be further substituted with carbamate (which may be further substituted with C₁-C₆ linear or branched alkyl));
amide optionally substituted with 1-2 groups independently selected from C₁-C₃ alkyl,
C₁-C₆ linear and branched alkylsulfonyl,
C₁-C₆ linear and branched alkoxy optionally substituted with 1-2 groups independently selected from hydroxy, oxo, C₃-C₆ cycloalkyl (which may be further substituted with carboxylic acid), and 3- to 6-membered heteroaryl;
4- to 6-membered heterocyclyl optionally substituted with 1-2 groups independently selected from halogen, oxo, hydroxy, amino, and C₁-C₃ alkyl (which may be further substituted with 1-3 groups independently selected from halogen),
C₃-C₆ cycloalkyl optionally substituted with 1-2 groups independently selected from amide, hydroxy, halogen, C₁-C₆ linear and branched alkyl (which may be further substituted with 1-3 groups independently selected from halogen), and carbamate (which may be further substituted with C₁-C₆ linear and branched alkyl), and
4- to 6-membered heteroaryl optionally substituted with 1-2 groups independently selected from amino, halogen, oxo, hydroxy, and C₁-C₆ linear and branched alkyl (which may be further substituted with 1-3 groups independently selected from halogen);
   and R⁵ is selected from hydrogen and C₁-C₃ linear or branched alkyl.
   18. The compound, deuterated derivative, or pharmaceutically acceptable salt according to Embodiment 4 or 5, wherein R is —NR⁵—C(O)R³, and wherein R³ is selected from:
hydrogen,
C₁-C₆ linear and branched alkyl optionally substituted with 1-4 groups independently selected from halogen, hydroxy, cyano, amido (which may be further substituted by 1-2 groups independently selected from C₁-C₃ alkyl), amino (which may be further substituted with C₁-C₃ alkylsulfonyl), carbamate (which may be further substituted with C₁-C₆ linear and branched alkyl), 4- to 6-membered heterocyclyl (which may be further substituted with 1-2 groups independently selected from halogen, oxo, and hydroxy), 4- to 6-membered heteroaryl (which may be further substituted with 1-2 groups independently selected from halogen, oxo, hydroxy, and C₁-C₃ alkyl), and C₃-C₆ cycloalkyl (which may be further substituted with carbamate (which may be further substituted with C₁-C₆ linear or branched alkyl));
amide optionally substituted with 1-2 groups independently selected from C₁-C₃ alkyl,
C₁-C₆ linear and branched alkylsulfonyl,
C₁-C₆ linear and branched alkoxy optionally substituted with 1-2 groups independently selected from hydroxy, oxo, C₃-C₆ cycloalkyl (which may be further substituted with carboxylic acid), and 3- to 6-membered heteroaryl;
4- to 6-membered heterocyclyl optionally substituted with 1-2 groups independently selected from halogen, oxo, hydroxy, and C₁-C₃ alkyl (which may be further substituted with 1-3 groups independently selected from halogen),
C₃-C₆ cycloalkyl optionally substituted with 1-2 groups independently selected from amide, hydroxy, halogen, C₁-C₆ linear and branched alkyl (which may be further substituted with 1-3 groups independently selected from halogen), and carbamate (which may be further substituted with C₁-C₆ linear and branched alkyl), and
4- to 6-membered heteroaryl optionally substituted with 1-2 groups independently selected from amino, halogen, oxo, hydroxy, and C₁-C₆ linear and branched alkyl (which may be further substituted with 1-3 groups independently selected from halogen);
   and R⁵ is selected from hydrogen and C₁-C₃ linear or branched alkyl.
   19. The compound, deuterated derivative, or pharmaceutically acceptable salt according to Embodiment 17 or 18, wherein R⁵ is hydrogen.
   20. The compound, deuterated derivative, or pharmaceutically acceptable salt according to any one of Embodiments 1-15, wherein R is —NR³R⁴, and wherein R³ and R⁴ are independently selected from:
4- to 6-membered heterocyclyl optionally substituted with 1-2 groups independently selected from hydroxy, halogen, and oxo,
C₁-C₃ alkyl optionally substituted with hydroxy, oxo, or halogen, and
hydrogen;
   or R³ and R⁴, together with the nitrogen atom to which they are attached, form a 4- to 10-membered heterocyclyl optionally substituted with 1-3 groups independently selected from oxo and C₁-C₃ alkyl.
   21. The compound, deuterated derivative, or pharmaceutically acceptable salt according to any one of Embodiments 1-5 and 8-15, wherein R is —OR³, and wherein R³ is selected from hydrogen and C₁-C₆ linear and branched alkyl.
   22. The compound, deuterated derivative, or pharmaceutically acceptable salt according to any one of Embodiments 1-3 and 8-15, wherein R is —OC(O)NR³R⁴, and wherein R³ is selected from:
C₁-C₆ linear and branched alkyl optionally substituted with 1-4 groups independently selected from halogen, hydroxy, amide, cyano, C₃-C₆ cycloalkyl (which may be further substituted with hydroxy or C₁-C₃ alkoxy), 4- to 6-membered heteroaryl (which may be further substituted with C₁-C₃ alkyl, or trifluoro substituted C₁-C₃ alkyl), and 4- to 6-membered heterocyclyl (which may be further substituted with 1-3 groups independently selected from oxo and hydroxy),
C₁-C₆ linear and branched alkoxy,
C₃-C₆ cycloalkyl optionally substituted with 1-2 groups independently selected from hydroxy, halogen, amide, C₁-C₃ alkyl (which may be further substituted with hydroxy or halogen), and C₁-C₃ alkoxy,
4- to 6-membered heterocyclyl optionally substituted with 1-2 groups independently selected from hydroxy, halogen, oxo, and C₁-C₃ alkyl, and
4- to 6-membered heteroaryl optionally substituted with 1-2 groups independently selected from hydroxy, halogen, and oxo,
or R³ and R⁴, together with the nitrogen atom to which they are attached, form a 4- to 10-membered heterocyclyl optionally substituted with 1-3 groups independently selected from oxo, hydroxy, and C₁-C₃ alkyl.
   23. The compound, deuterated derivative, or pharmaceutically acceptable salt according to Embodiment 4 or 5, wherein R is —OC(O)NR³R⁴, and wherein R³ is selected from:
   C₁-C₆ linear and branched alkyl optionally substituted with 1-4 groups independently selected from halogen, hydroxy, amide, cyano, C₃-C₆ cycloalkyl (which may be further substituted with hydroxy or C₁-C₃ alkoxy), 4- to 6-membered heteroaryl (which may be further substituted with C₁-C₃ alkyl, or trifluoro substituted C₁-C₃ alkyl), and 4- to 6-membered heterocyclyl (which may be further substituted with 1-2 groups independently selected from oxo and hydroxy),
   C₁-C₆ linear and branched alkoxy,
   C₃-C₆ cycloalkyl optionally substituted with 1-2 groups independently selected from hydroxy, halogen, amide, C₁-C₃ alkyl (which may be further substituted with hydroxy or halogen), and C₁-C₃ alkoxy,
   4- to 6-membered heterocyclyl optionally substituted with 1-2 groups independently selected from hydroxy, halogen, oxo, and C₁-C₃ alkyl, and
   4- to 6-membered heteroaryl optionally substituted with 1-2 groups independently selected from hydroxy, halogen, and oxo,
or R³ and R⁴, together with the nitrogen atom to which they are attached, form a 4- to 10-membered heterocyclyl optionally substituted with 1-3 groups independently selected from oxo, hydroxy, and C₁-C₃ alkyl.
   24. The compound, deuterated derivative, or pharmaceutically acceptable salt according to any one of Embodiments 1-5 and 8-15, wherein R is —NR⁵—SO₂R³, and wherein R³ is selected from:
   C₁-C₆ linear and branched alkyl optionally substituted with 1-2 groups independently selected from hydroxy, halogen, and 4- to 6-membered heterocyclyl (which may be further substituted with 1-2 groups independently selected from halogen, oxo, and hydroxy),
   4- to 6-membered heterocyclyl,
   4- to 6-membered heteroaryl optionally substituted with C₁-C₃ alkyl, and
   amino optionally substituted with 1-2 groups independently selected from C₁-C₃ alkyl.
      25. The compound, deuterated derivative, or pharmaceutically acceptable salt according to any one of Embodiments 1-5 and 8-15, wherein R is —C(O)OR³, and wherein R³ is selected from C₁-C₃ alkyl.
      26. The compound, deuterated derivative, or pharmaceutically acceptable salt according to any one of Embodiments 1-3 and 8-15, wherein R is —NR⁵C(O)NR³R⁴, and wherein R³ and R⁴ are independently selected from:
   C₁-C₆ linear and branched alkyl optionally substituted with 1-4 groups independently selected from halogen, hydroxy, oxo, cyano, amino (which may be further substituted with hydroxy), amido (which may be further substituted with hydroxy), sulfonic acid, aryl (optionally substituted with hydroxy), C₃-C₆ cycloalkyl (which may be further substituted 1-2 groups independently selected from hydroxy and C₁-C₃ hydroxyalkyl), and carboxylic acid,
   C₃-C₆ cycloalkyl optionally substituted with 1-2 groups independently selected from halogen, hydroxy, and C₁-C₆ linear and branched alkyl (which may be further substituted with hydroxy),
   4- to 6-membered heterocyclyl optionally substituted with 1-2 groups independently selected from halogen, oxo, and hydroxy, and
   C₁-C₆ linear and branched alkylsulfonyl;
      and R⁵ is selected from hydrogen and C₁-C₃ linear or branched alkyl.
      27. The compound, deuterated derivative, or pharmaceutically acceptable salt according to Embodiment 4 or 5, wherein R is —NR⁵C(O)NR³R⁴, and wherein R³ and R⁴ are independently selected from:
   C₁-C₆ linear and branched alkyl optionally substituted with 1-4 groups independently selected from halogen, hydroxy, oxo, cyano, amino, amido (which may be further substituted with hydroxy), sulfonic acid, aryl (optionally substituted with hydroxy), C₃-C₆ cycloalkyl (which may be further substituted 1-2 groups independently selected from hydroxy and C₁-C₃ hydroxyalkyl), and carboxylic acid,
   C₃-C₆ cycloalkyl optionally substituted with 1-2 groups independently selected from halogen, hydroxy, and C₁-C₆ linear and branched alkyl (which may be further substituted with hydroxy),
   4- to 6-membered heterocyclyl optionally substituted with 1-2 groups independently selected from halogen, oxo, and hydroxy, and
   C₁-C₆ linear and branched alkylsulfonyl;
      and R⁵ is selected from hydrogen and C₁-C₃ linear or branched alkyl.
      28. The compound, deuterated derivative, or pharmaceutically acceptable salt according to Embodiment 26 or 27, wherein R⁵ is hydrogen.
      29. The compound, deuterated derivative, or pharmaceutically acceptable salt according to any one of Embodiments 1-5 and 8-15, wherein:

R is

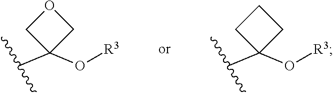

and

R³ is hydrogen.

30. A compound, deuterated derivative, or pharmaceutically acceptable salt selected from Compounds 1 to 527 (Table 1), deuterated derivatives thereof, or pharmaceutically acceptable salts of any of the foregoing.
31. A pharmaceutical composition comprising the compound, deuterated derivative, or pharmaceutically acceptable salt according to any one of Embodiments 1-30 and a pharmaceutically acceptable carrier.
32. A method of treating APOL1 mediated kidney disease comprising administering to a patient in need thereof the compound, deuterated derivative, or pharmaceutically acceptable salt according to any one of Embodiments 1-30 or the pharmaceutical composition according to
33. The compound, deuterated derivative, or pharmaceutically acceptable salt according to any one of Embodiments 1-30, or the pharmaceutical composition according to Embodiment 31, for use in treating APOL1 mediated kidney disease.
34. Use of a compound, deuterated derivative, or pharmaceutically acceptable salt according to any one of Embodiments 1-30 in the manufacture of a medicament for treating APOL1 mediated kidney disease.
35. The method, compound, deuterated derivative, pharmaceutically acceptable salt, or pharmaceutical composition for use, or use according to any one of Embodiments 32-34, wherein the APOL1 mediated kidney disease is selected from ESKD, NDKD, FSGS, HIV-associated nephropathy, sickle cell nephropathy, diabetic neuropathy, arterionephrosclerosis, lupus nephritis, microalbuminuria, and chronic kidney disease.
36. The method, compound, deuterated derivative, pharmaceutically acceptable salt, or pharmaceutical composition for use, or use according to Embodiment 35, wherein the APOL1 mediated kidney disease is FSGS.
37. The method, compound, deuterated derivative, pharmaceutically acceptable salt, or pharmaceutical composition for use, or use according to Embodiment 35, wherein the APOL1 mediated kidney disease is NDKD.
38. The method, compound, deuterated derivative, pharmaceutically acceptable salt, or pharmaceutical composition for use, or use according to Embodiment 35, wherein the APOL1 mediated kidney disease is ESKD.
39. The method, compound, deuterated derivative, pharmaceutically acceptable salt, or pharmaceutical composition for use, or use according to any one of Embodiments 32-38, wherein the APOL1 is associated with APOL1 genetic alleles chosen from homozygous G1: S342G:I384M and homozygous G2: N388del:Y389del.
40. The method, compound, deuterated derivative, pharmaceutically acceptable salt, or pharmaceutical composition for use, or use according to any one of Embodiments 32-38, wherein the APOL1 is associated with compound heterozygous G1: S342G:I384M and G2: N388del:Y389del APOL1 alleles.
41. A method of treating APOL1 mediated kidney disease comprising administering to a patient in need thereof a compound, deuterated derivative, or pharmaceutically acceptable salt selected from compounds of Formula II:

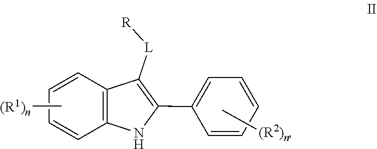

deuterated derivatives thereof, and pharmaceutically acceptable salts of any of the foregoing, wherein:
(i) R is selected from —C(O)NR³R⁴, —NR⁵C(O)R³, —NR⁵C(O)NR³R⁴, —NR³R⁴, —OR³, —NR⁵—SO₂R³, —OC(O)NR³R⁴, —C(O)OR³,

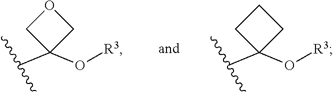

(ii) L is selected from divalent C₁-C₆ linear and branched alkyl (e.g., divalent C₁-C₆ linear and C₃-C₆ branched alkyl), divalent C₂-C₆ linear and branched alkenyl (e.g., divalent C₂-C₆ linear and C₃-C₆ branched alkenyl), divalent C₂-C₆ linear and branched alkynyl (e.g., divalent C₂-C₆ linear and C₃-C₆ branched alkynyl), and divalent 1- to 7-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;
   (iii) each R¹ is independently selected from:
halogen,
hydroxy,
thiol,
amino,
cyano,
C₁-C₆ linear, branched, and cyclic alkyl (e.g., C₁-C₆ linear, C₃-C₆ branched, and
C₃-C₆ cyclic alkyl) optionally substituted with 1-3 groups independently selected from hydroxy and halogen,
C₂-C₆ linear, branched, and cyclic alkenyl (e.g., C₂-C₆ linear, C₃-C₆ branched, and
C₃-C₆ cyclic alkenyl),
C₁-C₆ linear, branched, and cyclic alkoxy (e.g., C₁-C₆ linear, C₂-C₆ branched, and
C₂-C₆ cyclic alkoxy) optionally substituted with 1-3 groups independently selected from halogen,
C₁-C₆ linear, branched, and cyclic thioalkyl (e.g., C₁-C₆ linear, C₂-C₆ branched, and C₂-C₆ cyclic thioalkyl) optionally substituted with 1-3 groups independently selected from halogen, and
C₁-C₆ linear, branched, and cyclic aminoalkyl optionally substituted with 1-3 groups independently selected from halogen,
or two R′ groups, together with the carbon atoms to which they are attached, may form a C₄-C₈ cycloalkyl, aryl, or heteroaryl;
   (iv) each R² is independently selected from:
halogen,
hydroxy,
thiol,
amino,
cyano,
C₁-C₆ linear, branched, and cyclic alkyl (e.g., C₁-C₆ linear, C₃-C₆ branched, and
C₃-C₆ cyclic alkyl) optionally substituted with 1-3 groups independently selected from hydroxy and halogen,
C₂-C₄ linear, branched, and cyclic alkenyl (e.g., C₂-C₄ linear, C₃-C₄ branched, and
C₃-C₄ cyclic alkenyl),
C₁-C₆ linear, branched, and cyclic alkoxy (e.g., C₁-C₆ linear, C₂-C₆ branched, and
C₂-C₆ cyclic alkoxy) optionally substituted with 1-3 groups independently selected from halogen,
C₁-C₄ linear, branched, and cyclic thioalkyl (e.g., C₁-C₄ linear, C₂-C₄ branched, and C₂-C₄ cyclic thioalkyl) optionally substituted with 1-3 groups independently selected from halogen, and
C₁-C₄ linear, branched, and cyclic aminoalkyl optionally substituted with 1-3 groups independently selected from halogen,
   (v) each n is independently selected from 0, 1, 2, 3, and 4;
   (vi) R³ and R⁴ are independently selected from:
hydrogen,
C₁-C₆ linear and branched alkylsulfonyl (e.g., C₁-C₆ linear and C₂-C₆ branched alkyl sulfonyl),
C₂-C₆ linear and branched alkenyl (e.g., C₂-C₆ linear and C₃-C₆ branched alkenyl),
amino optionally substituted with 1-2 groups independently selected from C₁-C₆ linear and branched alkyl (e.g., C₁-C₆ linear and C₃-C₆ branched alkyl) and C₃-C₆ cycloalkyl,
amide optionally substituted with 1-2 groups independently selected from C₁-C₃ alkyl,
C₁-C₆ linear and branched alkoxy (e.g., C₁-C₆ linear and C₂-C₆ branched alkoxy) optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl (e.g., C₃-C₆ cyclic alkyl) optionally substituted with 1-2 groups independently selected from:
   halogen,
   hydroxy,
   oxo,
   amino optionally substituted with 1-2 groups independently selected from hydrogen and C₁-C₆ linear or branched alkyl (e.g., C₁-C₆ linear or C₃-C₆ branched alkyl),
   aryl optionally substituted with 1-2 groups independently selected from halogen,
   C₁-C₆ linear and branched alkyl groups (e.g., C₁-C₆ linear and C₃-C₆ branched alkyl groups) (which may be further substituted with 1-3 groups independently selected from hydroxy, oxo, halogen, and C₁-C₆ linear and branched alkoxy groups (e.g., C₁-C₆ linear and C₂-C₆ branched alkoxy groups)),
   carbamate optionally substituted with 1-2 groups independently selected from
C₁-C₆ linear and branched alkyl (e.g., C₁-C₆ linear and C₃-C₆ branched alkyl),
   C₁-C₆ linear and branched alkoxy (e.g., C₁-C₆ linear and C₂-C₆ branched alkoxy), and
   amide,
4- to 10-membered heterocyclyl optionally substituted with 1-3 groups independently selected from:
   halogen,
   oxo,
   hydroxy,
   amino, and
   C₁-C₆ linear and branched alkyl (e.g., C₁-C₆ linear and C₃-C₆ branched alkyl) (which may be further substituted with 1-2 groups independently selected from hydroxy, oxo, and C₁-C₆ linear and branched alkoxy (e.g., C₁-C₆ linear and C₂-C₆ branched alkoxy)),
aryl optionally substituted with 1-3 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy groups (e.g., C₁-C₆ linear and C₂-C₆ branched alkoxy groups)),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino), and
C₁-C₆ linear and branched alkyl groups (e.g., C₁-C₆ linear and C₃-C₆ branched alkyl groups), wherein the alkyl groups are optionally substituted with 1-4 groups independently selected from:
   amino groups optionally substituted with 1-2 groups independently selected from hydroxy, C₁-C₆ linear, branched, and cyclic alkyl (e.g., C₁-C₆ linear, C₃-C₆ branched, and C₃-C₆ cyclic alkyl) (which may be further substituted with 1-2 oxo), and C₁-C₆ linear and branched alkylsulfonyl (e.g., C₁-C₆ linear and C₂-C₆ branched alkylsulfonyl),
   hydroxy,
   oxo,
   cyano,
   carboxylic acid,
   sulfonic acid,
   —O-heteroaryl,
   carbamate optionally substituted with 1-2 groups independently selected from C₁-C₆ linear and branched alkyl (e.g., C₁-C₆ linear and C₃-C₆ branched alkyl),
   halogen,
   amido optionally substituted with 1-2 groups independently selected from hydroxy, C₁-C₆ linear, branched, and cyclic alkyl groups (e.g., C₁-C₆ linear, C₃-C₆ branched, and C₃-C₆ cyclic alkyl groups), and C₁-C₆ linear, branched, and cyclic hydroxyalkyl (e.g., C₁-C₆ linear, C₃-C₆ branched, and C₃-C₆ cyclic hydroxyalkyl groups),
   C₃-C₆ cyclic alkyl optionally substituted with 1-2 groups independently selected from halogen, hydroxy, C₁-C₆ linear and branched hydroxyalkyl (e.g., C₁-C₆ linear and C₃-C₆ branched hydroxyalkyl), C₁-C₆ linear and branched alkoxy (e.g., C₁-C₆ linear and C₂-C₆ branched alkoxy), and carbamate (which may be further substituted with 1-2 groups independently selected from C₁-C₆ linear and branched alkyl (e.g., C₁-C₆ linear and C₃-C₆ branched alkyl)),
   C₁-C₆ linear and branched alkynyl (e.g., C₂-C₆ linear and branched alkynyl, e.g., C₂-C₆ linear and C₃-C₆ branched alkynyl),
   C₁-C₆ linear and branched alkoxy (e.g., C₁-C₆ linear and C₂-C₆ branched alkoxy) optionally substituted with 1-2 hydroxy,
   C₁-C₆ linear and branched alkylsulfonyl (e.g., C₁-C₆ linear and C₂-C₆ branched alkylsulfonyl),
   aryl optionally substituted with 1-2 groups independently selected from halogen groups, hydroxy, and C₁-C₆ linear and branched alkyl groups (e.g., C₁-C₆ linear and C₃-C₆ branched alkyl groups) (which may be further substituted with 1-2 groups independently selected from hydroxy and C₁-C₆ linear and branched alkoxy groups (e.g., C₁-C₆ linear and C₂-C₆ branched alkoxy groups)),
   carbonyl-(4-methylpiperazin-1-yl),
   carbonyl-(N-morpholino),
   4- to 10-membered heterocyclyl optionally substituted with 1-3 groups independently selected from halogen, oxo, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy (e.g., C₁-C₆ linear and C₂-C₆ branched alkoxy)), and
   4- to 10-membered heteroaryl optionally substituted with 1-3 groups independently selected from halogen, oxo, hydroxy, and C₁-C₆ linear, branched, and cyclic alkyl (e.g., C₁-C₆ linear, C₃-C₆ branched, and C₃-C₆ cyclic alkyl) (which may be further substituted with 1-3 groups independently selected from halogen, hydroxy, and C₁-C₆ linear and branched alkoxy (e.g., C₁-C₆ linear and C₂-C₆ branched alkoxy)),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl (e.g., C₁-C₆ linear, C₃-C₆ branched, and C₃-C₆ cyclic alkyl groups),
halogen,
hydroxy,
oxo,
C₁-C₆ linear, branched, and cyclic alkyl (e.g., C₁-C₆ linear, C₃-C₆ branched, and C₃-C₆ cyclic alkyl groups) optionally substituted with 1-2 groups independently selected from hydroxy, amino, C₁-C₆ linear, branched, and cyclic alkoxy groups (e.g., C₁-C₆ linear, C₂-C₆ branched, and C₂-C₆ cyclic alkoxy groups), and carbamate (which may be further substituted with 1-2 groups independently selected from C₁-C₆ linear and branched alkyl (e.g., C₁-C₆ linear and C₃-C₆ branched alkyl)),
amide optionally substituted with 1-2 groups independently selected from C₁-C₆ linear and branched alkyl (e.g., C₁-C₆ linear and C₃-C₆ branched alkyl),
carbamate optionally substituted with 1-2 groups independently selected from
C₁-C₆ linear and branched alkyl (e.g., C₁-C₆ linear and C₃-C₆ branched alkyl),
carboxamide optionally substituted with 1-2 groups independently selected from C₁-C₆ linear and branched alkyl (e.g., C₁-C₆ linear and C₃-C₆ branched alkyl),
C₁-C₆ linear and branched alkoxy groups (e.g., C₁-C₆ linear and C₂-C₆ branched alkoxy groups) optionally substituted with 1-2 groups independently selected from C₁-C₆ linear, branched, and cyclic alkyl (e.g., C₁-C₆ linear, C₃-C₆ branched, and C₃-C₆ cyclic alkyl) and heterocyclyl,
4- to 10-membered heterocyclyl optionally substituted with 1-2 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy (e.g., C₁-C₆ linear and C₂-C₆ branched alkoxy)), and
4- to 10-membered heteroaryl optionally substituted with 1-2 groups independently selected from halogen, hydroxy, and C₁-C₆ linear and branched alkyl (e.g., C₁-C₆ linear and C₃-C₆ branched alkyl) (which may be further substituted with 1-2 groups independently selected from hydroxy and C₁-C₆ linear and branched alkoxy groups (e.g., C₁-C₆ linear and C₂-C₆ branched alkoxy groups));

and

(vii) R⁵ is selected from hydrogen and C₁-C₆ linear or branched alkyl (e.g., C₁-C₆ linear or C₃-C₆ branched alkyl);
with the provisos that

(1) the compound is not

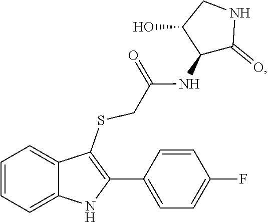 [SMILES: O=C(CSc1c(-c2ccc(F)cc2)[nH]c2ccccc12)N[C@@H]1C(=O)NC[C@H]1O]

and

(2) when L is a divalent C₂ linear alkyl optionally substituted with 1-2 groups independently selected from methyl, halogen, and hydroxy and R is —NR³R⁴, then R³ and R⁴ are not

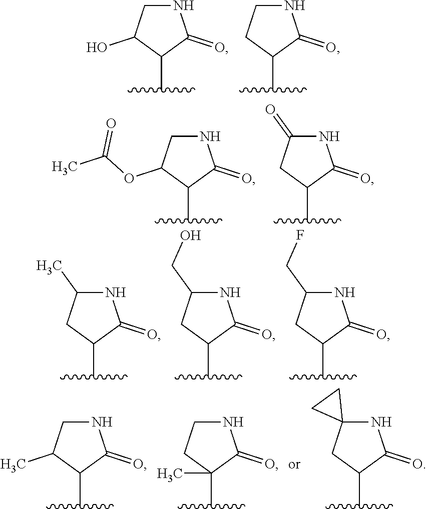

42. The method according to Embodiment 41, wherein the APOL1 mediated kidney disease is chosen from ESKD, NDKD, FSGS, HIV-associated nephropathy, arterionephrosclerosis, lupus nephritis, microalbuminuria, and chronic kidney disease.
43. The method according to Embodiment 41, wherein the APOL1 mediated kidney disease is FSGS.
44. The method according to Embodiment 41, wherein the APOL1 mediated kidney disease is NDKD.
45. The method according to Embodiment 41, wherein the APOL1 mediated kidney disease is ESKD.
46. The method according to any one of Embodiments 41-45, wherein the APOL1 mediated kidney disease is associated with APOL1 genetic alleles chosen from homozygous G1: S342G:I384M and homozygous G2: N388del:Y389del.
47. The method according to any one of Embodiments 41-45, wherein the APOL1 is associated with compound heterozygous G1: S342G:I384M and G2: N388del:Y389del APOL1 alleles.
48. A method of inhibiting APOL1 activity comprising contacting said APOL1 with a compound selected from Formula II, a deuterated derivative thereof, or a pharmaceutically acceptable salt of any of the foregoing.
49. The method according to Embodiment 48, wherein the APOL1 is associated with APOL1 genetic alleles chosen from homozygous G1: S342G:I384M and homozygous G2: N388del:Y389del.
50. The method according to Embodiment 48, wherein the APOL1 is associated with homozygous G1: S342G:I384M APOL1 alleles.
51. The method according to Embodiment 48, wherein the APOL1 is associated with compound heterozygous G1: S342G:I384M and G2: N388del:Y389del APOL1 alleles.
52. A compound, deuterated derivative, or pharmaceutically acceptable salt selected from compounds of Formula II, deuterated derivatives thereof, and pharmaceutically acceptable salts of any of the foregoing, for use in treating APOL1 mediated kidney disease.
53. The compound, deuterated derivative, or pharmaceutically acceptable salt for use according to Embodiment 52, wherein the APOL1 mediated kidney disease is chosen from ESKD, NDKD, FSGS, HIV-associated nephropathy, arterionephrosclerosis, lupus nephritis, microalbuminuria, and chronic kidney disease.
54. The method according to Embodiment 52, wherein the APOL1 mediated kidney disease is FSGS.
55. The method according to Embodiment 52, wherein the APOL1 mediated kidney disease is NDKD.
56. The method according to Embodiment 52, wherein the APOL1 mediated kidney disease is ESKD.
57. The compound, deuterated derivative, or pharmaceutically acceptable salt for use according to any one of Embodiments 52-56, wherein the APOL1 is associated with APOL1 genetic alleles chosen from homozygous G1: S342G:I384M and homozygous G2: N388del:Y389del.
58. The compound, deuterated derivative, or pharmaceutically acceptable salt for use according to Embodiments 52-56, wherein the APOL1 is associated with compound heterozygous G1: S342G:I384M and G2: N388del:Y389del APOL1 alleles.
59. Use of a compound, deuterated derivative, or pharmaceutically acceptable salt selected from compounds of Formula II, deuterated derivatives thereof, and pharmaceutically acceptable salts of any of the foregoing, in the manufacture of a medicament for treating APOL1 mediated kidney disease.
60. The use according to Embodiment 59, wherein the APOL1 mediated kidney disease is selected from ESKD, NDKD, FSGS, HIV-associated nephropathy, arterionephrosclerosis, lupus nephritis, microalbuminuria, and chronic kidney disease.
61. The use according to Embodiment 59, wherein the APOL1 mediated kidney disease is FSGS.
62. The use according to Embodiment 59, wherein the APOL1 mediated kidney disease is NDKD.
63. The use according to Embodiment 59, wherein the APOL1 mediated kidney disease is ESKD.
64. The use according to any one of Embodiments 59-63, wherein the APOL1 is associated with APOL1 genetic alleles chosen from homozygous G1: S342G:I384M and homozygous G2: N388del:Y389del.
65. The use according to any one of Embodiments 59-63, wherein the APOL1 is associated with compound heterozygous G1: S342G:I384M and G2: N388del:Y389del APOL1 alleles.
66. The method, compound, deuterated derivative, or pharmaceutically acceptable salt for use, or use according to any one of Embodiments 41-65, wherein L is selected from divalent C₁-C₆ linear and branched alkyl, divalent C₂-C₆ linear and branched alkenyl, divalent C₂-C₆ linear and branched alkynyl, and divalent 1- to 6-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino.
   67. The method, compound, deuterated derivative, or pharmaceutically acceptable salt for use, or use according to any one of Embodiments 41-66, wherein each R² is independently selected from:
halogen,
hydroxy,
thiol,
amino,
cyano,
C₁-C₄ linear, branched, and cyclic alkyl optionally substituted with 1-3 groups independently selected from hydroxy and halogen,
C₂-C₄ linear, branched, and cyclic alkenyl,
C₁-C₄ linear, branched, and cyclic alkoxy optionally substituted with 1-3 groups independently selected from halogen,
C₁-C₄ linear, branched, and cyclic thioalkyl optionally substituted with 1-3 groups independently selected from halogen, and
C₁-C₄ linear, branched, and cyclic aminoalkyl optionally substituted with 1-3 groups independently selected from halogen.
   68. The method, compound, deuterated derivative, or pharmaceutically acceptable salt for use, or use according to any one of Embodiments 41-67, wherein R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl and C₃-C₆ cycloalkyl,
amide optionally substituted with 1-2 groups independently selected from C₁-C₃ alkyl, and
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl optionally substituted with 1-2 groups independently selected from:
   halogen,
   hydroxy,
   oxo,
   amino optionally substituted with 1-2 groups independently selected from hydrogen and C₁-C₆ linear or branched alkyl,
   aryl optionally substituted with 1-2 groups independently selected from halogen,
   C₁-C₆ linear and branched alkyl groups (which may be further substituted with 1-3 groups independently selected from hydroxy, oxo, halogen, and C₁-C₆ linear and branched alkoxy groups),
   carbamate optionally substituted with 1-2 groups independently selected from
C₁-C₆ linear and branched alkyl,
   C₁-C₆ linear and branched alkoxy, and
   amide,
4- to 10-membered heterocyclyl optionally substituted with 1-3 groups independently selected from:
   halogen,
   oxo,
   hydroxy, and
   C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, and C₁-C₆ linear and branched alkyl (which may be further substituted with one or two groups independently selected from hydroxy and C₁-C₆ linear and branched alkoxy groups),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino),
C₁-C₆ linear and branched alkyl groups, wherein the alkyl groups are optionally substituted with 1-4 groups independently selected from:
   amino groups optionally substituted with 1-2 groups independently selected from C₁-C₆ linear, branched, and cyclic alkyl (which may be further substituted with 1-2 oxo), and C₁-C₆ linear and branched alkylsulfonyl,
   hydroxy,
   oxo,
   cyano,
   carboxylic acid,
   sulfonic acid,
   —O-heteroaryl,
   carbamate optionally substituted with 1-2 groups independently selected from
C₁-C₆ linear and branched alkyl,
   halogen,
   amido optionally substituted with 1-2 groups independently selected from hydroxy, C₁-C₆ linear, branched, and cyclic alkyl groups and C₁-C₆ linear, branched, and cyclic hydroxyalkyl,
   C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
   C₁-C₆ linear and branched alkynyl,
   C₁-C₆ linear and branched alkoxy optionally substituted with 1-2 hydroxy,
   C₁-C₆ linear and branched alkylsulfonyl,
   aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
   carbonyl-(4-methylpiperazin-1-yl),
   carbonyl-(N-morpholino),
   4- to 10-membered heterocyclyl optionally substituted with 1-2 groups independently selected from halogen, oxo, hydroxy, and C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-3 groups independently selected from halogen, oxo, hydroxy, and C₁-C₆ linear,
branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl,
halogen,
hydroxy,
oxo,
C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
amide optionally substituted with 1-2 groups independently selected from C₁-C₆ linear and branched alkyl,
carbamate optionally substituted with 1-2 groups independently selected from
C₁-C₆ linear and branched alkyl,
carboxamide optionally substituted with 1-2 groups independently selected from
C₁-C₆ linear and branched alkyl,
C₁-C₆ linear and branched alkoxy groups optionally substituted with 1-2 groups independently selected from C₁-C₆ linear, branched, and cyclic alkyl, and heterocyclyl,
4- to 10-membered heterocyclyl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups).
   69. The method, compound, deuterated derivative, or pharmaceutically acceptable salt for use, or use according to any one of Embodiments 41-68, wherein:

L is selected from divalent C₁-C₆ linear and branched alkyl, divalent C₂-C₆ linear and branched alkenyl, divalent C₂-C₆ linear and branched alkynyl, and divalent 1- to 6-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;

each R² is independently selected from:
halogen,
hydroxy,
thiol,
amino,
cyano,
C₁-C₄ linear, branched, and cyclic alkyl optionally substituted with 1-3 groups independently selected from hydroxy and halogen,
C₂-C₄ linear, branched, and cyclic alkenyl,
C₁-C₄ linear, branched, and cyclic alkoxy optionally substituted with 1-3 groups independently selected from halogen,
C₁-C₄ linear, branched, and cyclic thioalkyl optionally substituted with 1-3 groups independently selected from halogen, and
C₁-C₄ linear, branched, and cyclic aminoalkyl optionally substituted with 1-3 groups independently selected from halogen; and

R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl and C₃-C₆ cycloalkyl,
amide optionally substituted with 1-2 groups independently selected from C₁-C₃ alkyl, and
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl optionally substituted with 1-2 groups independently selected from:
   halogen,
   hydroxy,
   oxo,
   amino optionally substituted with 1-2 groups independently selected from hydrogen and C₁-C₆ linear or branched alkyl,
   aryl optionally substituted with 1-2 groups independently selected from halogen,
   C₁-C₆ linear and branched alkyl groups (which may be further substituted with 1-3 groups independently selected from hydroxy, oxo, halogen, and C₁-C₆ linear and branched alkoxy groups),
   carbamate optionally substituted with 1-2 groups independently selected from
C₁-C₆ linear and branched alkyl,
   C₁-C₆ linear and branched alkoxy, and
   amide,
4- to 10-membered heterocyclyl optionally substituted with 1-3 groups independently selected from:
   halogen,
   oxo,
   hydroxy, and
   C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, and C₁-C₆ linear and branched alkyl (which may be further substituted with one or two groups independently selected from hydroxy and C₁-C₆ linear and branched alkoxy groups),
4- to 10-membered heteroaryl optionally substituted with 1-3 groups independently selected from:
   amino,
   hydroxy,
   oxo,
   halogen, and
   C₁-C₆ linear alkyl (which may be further substituted with 1-3 groups independently selected from halogen and amino),
C₁-C₆ linear and branched alkyl groups, wherein the alkyl groups are optionally substituted with 1-4 groups independently selected from:
   amino groups optionally substituted with 1-2 groups independently selected from C₁-C₆ linear, branched, and cyclic alkyl (which may be further substituted with 1-2 oxo), and C₁-C₆ linear and branched alkylsulfonyl,
   hydroxy,
   oxo,
   cyano,
   carboxylic acid,
   sulfonic acid,
   —O-heteroaryl,
   carbamate optionally substituted with 1-2 groups independently selected from
C₁-C₆ linear and branched alkyl,
   halogen,
   amido optionally substituted with 1-2 groups independently selected from hydroxy, C₁-C₆ linear, branched, and cyclic alkyl groups and C₁-C₆ linear, branched, and cyclic hydroxyalkyl,
   C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
   C₁-C₆ linear and branched alkynyl,
   C₁-C₆ linear and branched alkoxy optionally substituted with 1-2 hydroxy,
   C₁-C₆ linear and branched alkylsulfonyl,
   aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
   carbonyl-(4-methylpiperazin-1-yl),
   carbonyl-(N-morpholino),
   4- to 10-membered heterocyclyl optionally substituted with 1-2 groups independently selected from halogen, oxo, hydroxy, and C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-3 groups independently selected from halogen, oxo, hydroxy, and C₁-C₆ linear,
branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl,
halogen,
hydroxy,
oxo,
C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
amide optionally substituted with 1-2 groups independently selected from C₁-C₆ linear and branched alkyl,
carbamate optionally substituted with 1-2 groups independently selected from
C₁-C₆ linear and branched alkyl,
carboxamide optionally substituted with 1-2 groups independently selected from
C₁-C₆ linear and branched alkyl,
C₁-C₆ linear and branched alkoxy groups optionally substituted with 1-2 groups independently selected from C₁-C₆ linear, branched, and cyclic alkyl, and heterocyclyl,
4- to 10-membered heterocyclyl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups).
   70. The method, compound, deuterated derivative, or pharmaceutically acceptable salt for use, or use according to any one of Embodiments 41-69, wherein each R¹ is independently selected from halogen, hydroxy, amino, C₁-C₆ linear and branched alkyl (optionally substituted with 1-3 groups independently selected from hydroxy and halogen), C₃-C₆ cycloalkyl, and C₁-C₆ linear and branched alkoxy (optionally substituted with 1-3 groups independently selected from halogen).
   71. The method, compound, deuterated derivative, or pharmaceutically acceptable salt for use, or use according to any one of Embodiments 41-66, 68, and 70, wherein each R² is independently selected from halogen, hydroxy, amino, cyano, C₁-C₆ linear and branched alkyl (optionally substituted with 1-3 groups independently selected from hydroxy and halogen), and C₁-C₆ linear and branched alkoxy (optionally substituted with 1-3 groups independently selected from halogen).
   72. The method, compound, deuterated derivative, or pharmaceutically acceptable salt for use, or use according to Embodiment 69, wherein each R² is independently selected from halogen, hydroxy, amino, cyano, C₁-C₄ linear and branched alkyl (optionally substituted with 1-3 groups independently selected from hydroxy and halogen), and C₁-C₄ linear and branched alkoxy (optionally substituted with 1-3 groups independently selected from halogen).
   73. The method, compound, deuterated derivative, or pharmaceutically acceptable salt for use, or use according to any one of Embodiments 41-72, wherein each R¹ and/or R² is fluorine.
   74. The method, compound, deuterated derivative, or pharmaceutically acceptable salt for use, or use according to any one of Embodiments 41-73, wherein each n is selected from 0, 1, and 2.
   75. The method, compound, deuterated derivative, or pharmaceutically acceptable salt for use, or use according to any one of Embodiments 41-65, 67, 68, and 70-74, wherein L is selected from divalent C₁-C₆ linear and branched alkyl and divalent C₁-C₆ linear and branched thioalkyl, wherein the divalent alkyl and divalent thioalkyl are optionally substituted with 1-2 groups independently selected from halogen.
   76. The method, compound, deuterated derivative, or pharmaceutically acceptable salt for use, or use according to Embodiment 75, wherein L is selected from divalent C₁-C₃ linear and branched alkyl, and divalent C₁-C₃ linear and branched thioalkyl, wherein the divalent alkyl and divalent thioalkyl are optionally substituted with 1-2 groups independently selected from halogen.
   77. The method, compound, deuterated derivative, or pharmaceutically acceptable salt for use, or use according to Embodiment 69, wherein L is selected from divalent C₁-C₆ linear and branched alkyl and divalent C₁-C₅ linear and branched thioalkyl, wherein the divalent alkyl and divalent thioalkyl are optionally substituted with 1-2 groups independently selected from halogen.
   78. The method, compound, deuterated derivative, or pharmaceutically acceptable salt for use, or use according to any one of Embodiments 41-77, wherein R is —C(O)NR³R⁴, and wherein R³ and R⁴ are independently selected from:
hydrogen,
C₁-C₆ linear and branched alkylsulfonyl,
C₁-C₆ linear and branched alkoxy optionally substituted with 1-2 groups independently selected from hydroxy and oxo;
C₃-C₆ cyclic alkyl optionally substituted with 1-2 groups independently selected from:
   halogen,
   hydroxy,
   oxo,
   amino,
   aryl optionally substituted with 1-2 groups independently selected from halogen,
   C₁-C₆ linear and branched alkyl groups (which may be further substituted with 1-3 groups independently selected from hydroxy and halogen),
   carbamate optionally substituted with 1-2 groups independently selected from
C₁-C₆ linear and branched alkyl,
   C₁-C₆ linear and branched alkoxy, and
   amido groups,
4- to 10-membered heterocyclyl optionally substituted with 1-3 groups independently selected from:
   oxo,
   hydroxy,
   C₁-C₆ linear and branched alkyl (which may be further substituted with 1-2 groups independently selected from hydroxy, oxo, and C₁-C₆ linear and branched alkoxy),
4- to 10-membered heteroaryl optionally substituted with 1-3 groups independently selected from:
   hydroxy,
   oxo,
   halogen, and
   C₁-C₆ linear alkyl (which may be further substituted with 1-3 groups independently selected from halogen),
C₁-C₆ linear and branched alkyl groups, wherein the alkyl groups are optionally substituted with 1-4 groups independently selected from:
   amino groups optionally substituted with 1-2 groups independently selected from C₁-C₆ linear, branched, and cyclic alkyl,
   hydroxy,
   oxo,
   cyano,
   carbamate optionally substituted with 1-2 groups independently selected from
C₁-C₆ linear and branched alkyl,
   halogen,
   amido,
   C₃-C₆ cyclic alkyl optionally substituted with 1-2 hydroxy,
   4- to 10-membered heterocyclyl optionally substituted with 1-2 groups independently selected from oxo, hydroxy, and C₁-C₆ linear and branched alkyl (which may be further substituted with 1-2 hydroxy), and
   4- to 10-membered heteroaryl optionally substituted with 1-3 groups independently selected from halogen and C₁-C₆ linear, branched, and cyclic alkyl (which may be further substituted with 1-3 groups independently selected from halogen),
or R³ and R⁴, together with the nitrogen atom to which they are attached, form a 4- to 10-membered heterocyclyl or heteroaryl optionally substituted with 1-3 groups independently selected from:
amino,
halogen,
hydroxy,
C₁-C₆ linear, branched, and cyclic alkyl optionally substituted with 1-2 groups independently selected from hydroxy, amino, and carbamate (which may be further substituted with 1-2 groups independently selected from C₁-C₆ linear and branched alkyl), and
carbamate optionally substituted with 1-2 groups independently selected from
C₁-C₆ linear and branched alkyl.
   79. The method, compound, deuterated derivative, or pharmaceutically acceptable salt for use, or use according to any one of Embodiments 41-67 and 70-77, wherein R is —NR⁵—C(O)R³, and wherein R³ is selected from:
hydrogen,
C₁-C₆ linear and branched alkyl optionally substituted with 1-4 groups independently selected from halogen hydroxy, cyano, amide (which may be further substituted by 1-2 groups independently selected from C₁-C₃ alkyl), amino (which may be further substituted with C₁-C₃ alkylsulfonyl), carbamate (which may be further substituted with C₁-C₆ linear and branched alkyl), 4- to 6-membered heterocyclyl (which may be further substituted with 1-2 groups independently selected from halogen, oxo, and hydroxy), 4- to 6-membered heteroaryl (which may be further substituted with 1-2 groups independently selected from halogen, oxo, hydroxy, and C₁-C₃ alkyl), and C₃-C₆ cycloalkyl (which may be further substituted with carbamate (which may be further substituted with C₁-C₆ linear or branched alkyl));
amide optionally substituted with 1-2 groups independently selected from C₁-C₃ alkyl,
C₁-C₆ linear and branched alkylsulfonyl,
C₁-C₆ linear and branched alkoxy optionally substituted with 1-2 groups independently selected from hydroxy, oxo, C₃-C₆ cycloalkyl (which may be further substituted with carboxylic acid), and 3- to 6-membered heteroaryl;
4- to 6-membered heterocyclyl optionally substituted with 1-2 groups independently selected from halogen, oxo, hydroxy, amino, and C₁-C₃ alkyl (which may be further substituted with 1-3 groups selected from halogen),
C₃-C₆ cycloalkyl optionally substituted with 1-2 groups independently selected from amide, hydroxy, halogen, C₁-C₆ linear and branched alkyl (which may be further substituted with 1-3 groups selected from halogen), and carbamate (which may be further substituted with C₁-C₆ linear and branched alkyl), and
4- to 6-membered heteroaryl optionally substituted with 1-2 groups independently selected from amino, halogen, oxo, hydroxy, and C₁-C₆ linear and branched alkyl (which may be further substituted with 1-3 groups selected from halogen);
and R⁵ is selected from hydrogen and linear or branched C₁-C₃ alkyl.
   80. The method, compound, deuterated derivative, or pharmaceutically acceptable salt for use, or use according to Embodiment 69, wherein R is —NR⁵—C(O)R³, and wherein R³ is selected from:
hydrogen,
C₁-C₆ linear and branched alkyl optionally substituted with 1-4 groups independently selected from halogen, hydroxy, cyano, amido (which may be further substituted by 1-2 groups independently selected from C₁-C₃ alkyl), amino (which may be further substituted with C₁-C₃ alkylsulfonyl), carbamate (which may be further substituted with C₁-C₆ linear and branched alkyl), 4- to 6-membered heterocyclyl (which may be further substituted with 1-2 groups independently selected from halogen, oxo, and hydroxy), 4- to 6-membered heteroaryl (which may be further substituted with 1-2 groups independently selected from halogen, oxo, hydroxy, and C₁-C₃ alkyl), and C₃-C₆ cycloalkyl (which may be further substituted with carbamate (which may be further substituted with C₁-C₆ linear or branched alkyl));
amide optionally substituted with 1-2 groups independently selected from C₁-C₃ alkyl,
C₁-C₆ linear and branched alkylsulfonyl,
C₁-C₆ linear and branched alkoxy optionally substituted with 1-2 groups independently selected from hydroxy, oxo, C₃-C₆ cycloalkyl (which may be further substituted with carboxylic acid), and 3- to 6-membered heteroaryl;
4- to 6-membered heterocyclyl optionally substituted with 1-2 groups independently selected from halogen, oxo, hydroxy, and C₁-C₃ alkyl (which may be further substituted with 1-3 groups independently selected from halogen),
C₃-C₆ cycloalkyl optionally substituted with 1-2 groups independently selected from amide, hydroxy, halogen, C₁-C₆ linear and branched alkyl (which may be further substituted with 1-3 groups independently selected from halogen), and carbamate (which may be further substituted with C₁-C₆ linear and branched alkyl), and
4- to 6-membered heteroaryl optionally substituted with 1-2 groups independently selected from amino, halogen, oxo, hydroxy, and C₁-C₆ linear and branched alkyl (which may be further substituted with 1-3 groups independently selected from halogen);
   and R⁵ is selected from hydrogen and C₁-C₃ linear or branched alkyl.
   81. The method, compound, deuterated derivative, or pharmaceutically acceptable salt for use, or use according to Embodiment 79 or 80, wherein R⁵ is hydrogen.
   82. The method, compound, deuterated derivative, or pharmaceutically acceptable salt for use, or use according to any one of embodiments 41-77, wherein R is —NR³R⁴, and wherein R³ and R⁴ are independently selected from:
4- to 6-membered heterocyclyl optionally substituted with 1-2 groups independently selected from hydroxy, halogen, and oxo,
C₁-C₃ alkyl optionally substituted with hydroxy, oxo, or halogen, and
hydrogen;
or R³ and R⁴, together with the nitrogen atom to which they are attached, form a 4- to 10-membered heterocyclyl optionally substituted with 1-3 groups independently selected from oxo and C₁-C₃ alkyl.
   83. The method, compound, deuterated derivative, or pharmaceutically acceptable salt for use, or use according to any one of embodiments 41-77, wherein R is —OR³, and wherein R³ is selected from hydrogen and C₁-C₆ linear and branched alkyl.
   84. The method, compound, deuterated derivative, or pharmaceutically acceptable salt for use, or use according to any one of Embodiments 41-67 and 70-77, wherein R is —OC(O)NR³R⁴, and wherein R³ is selected from:
   C₁-C₆ linear and branched alkyl optionally substituted with 1-4 groups independently selected from halogen, hydroxy, amide, cyano, C₃-C₆ cycloalkyl (which may be further substituted with hydroxy or C₁-C₃ alkoxy), 4- to 6-membered heteroaryl (which may be further substituted with C₁-C₃ alkyl, or trifluoro substituted C₁-C₃ alkyl), and 4- to 6-membered heterocyclyl (which may be further substituted with 1-3 groups independently selected from oxo and hydroxy),
   C₁-C₆ linear and branched alkoxy,
   C₃-C₆ cycloalkyl optionally substituted with 1-2 groups independently selected from hydroxy, halogen, amide, C₁-C₃ alkyl (which may be further substituted with hydroxy or halogen), and C₁-C₃ alkoxy,
   4- to 6-membered heterocyclyl optionally substituted with 1-2 groups independently selected from hydroxy, halogen, oxo, and C₁-C₃ alkyl, and
   4- to 6-membered heteroaryl optionally substituted with 1-2 groups independently selected from hydroxy, halogen, and oxo,
or R³ and R⁴, together with the nitrogen atom to which they are attached, form a 4- to 10-membered heterocyclyl optionally substituted with 1-3 groups independently selected from oxo, hydroxy, and C₁-C₃ alkyl.
   85. The method, compound, deuterated derivative, or pharmaceutically acceptable salt for use, or use according to Embodiment 69, wherein R is —OC(O)NR³R⁴, and wherein R³ is selected from:
   C₁-C₆ linear and branched alkyl optionally substituted with 1-4 groups independently selected from halogen, hydroxy, amide, cyano, C₃-C₆ cycloalkyl (which may be further substituted with hydroxy or C₁-C₃ alkoxy), 4- to 6-membered heteroaryl (which may be further substituted with C₁-C₃ alkyl, or trifluoro substituted C₁-C₃ alkyl), and 4- to 6-membered heterocyclyl (which may be further substituted with 1-2 groups independently selected from oxo and hydroxy),
   C₁-C₆ linear and branched alkoxy,
   C₃-C₆ cycloalkyl optionally substituted with 1-2 groups independently selected from hydroxy, halogen, amide, C₁-C₃ alkyl (which may be further substituted with hydroxy or halogen), and C₁-C₃ alkoxy,
   4- to 6-membered heterocyclyl optionally substituted with 1-2 groups independently selected from hydroxy, halogen, oxo, and C₁-C₃ alkyl, and
   4- to 6-membered heteroaryl optionally substituted with 1-2 groups independently selected from hydroxy, halogen, and oxo,
or R³ and R⁴, together with the nitrogen atom to which they are attached, form a 4- to 10-membered heterocyclyl optionally substituted with 1-3 groups independently selected from oxo, hydroxy, and C₁-C₃ alkyl.
   86. The method, compound, deuterated derivative, or pharmaceutically acceptable salt for use, or use according to any one of Embodiments 41-77, wherein R is —NR⁵—SO₂R³, and wherein R³ is selected from:
   C₁-C₆ linear and branched alkyl optionally substituted with 1-2 groups independently selected from hydroxy, halogen, and 4- to 6-membered heterocyclyl (which may be further substituted with 1-2 groups independently selected from halogen, oxo, and hydroxy),
   4- to 6-membered heterocyclyl,
   4- to 6-membered heteroaryl optionally substituted with C₁-C₃ alkyl, and
   amino optionally substituted with 1-2 groups independently selected from C₁-C₃ alkyl.
      87. The method, compound, deuterated derivative, or pharmaceutically acceptable salt for use, or use according to any one of Embodiments 41-77, wherein R is —C(O)OR³, and wherein R³ is selected from C₁-C₃ alkyl.
      88. The method, compound, deuterated derivative, or pharmaceutically acceptable salt for use, or use according to any one of Embodiments 41-67 and 70-77, wherein R is —NR⁵C(O)NR³R⁴, and wherein R³ and R⁴ are independently selected from:
   C₁-C₆ linear and branched alkyl optionally substituted with 1-4 groups independently selected from halogen, hydroxy, oxo, cyano, amino (which may be further substituted with hydroxy), amido (which may be further substituted with hydroxy), sulfonic acid, aryl (optionally substituted with hydroxy), C₃-C₆ cycloalkyl (which may be further substituted 1-2 groups independently selected from hydroxy and C₁-C₃ hydroxyalkyl), and carboxylic acid,
   C₃-C₆ cycloalkyl optionally substituted with 1-2 groups independently selected from halogen, hydroxy, and C₁-C₆ linear and branched alkyl (which may be further substituted with hydroxy),
   4- to 6-membered heterocyclyl optionally substituted with 1-2 groups independently selected from halogen, oxo, and hydroxy, and
   C₁-C₆ linear and branched alkylsulfonyl;
      and R⁵ is selected from hydrogen and C₁-C₃ linear or branched alkyl.
      89. The method, compound, deuterated derivative, or pharmaceutically acceptable salt for use, or use according to Embodiment 69, wherein R is —NR⁵C(O)NR³R⁴, and wherein R³ and R⁴ are independently selected from:
   C₁-C₆ linear and branched alkyl optionally substituted with 1-4 groups independently selected from halogen, hydroxy, oxo, cyano, amino, amido (which may be further substituted with hydroxy), sulfonic acid, aryl (optionally substituted with hydroxy), C₃-C₆ cycloalkyl (which may be further substituted 1-2 groups independently selected from hydroxy and C₁-C₃ hydroxyalkyl), and carboxylic acid,
   C₃-C₆ cycloalkyl optionally substituted with 1-2 groups independently selected from halogen, hydroxy, and C₁-C₆ linear and branched alkyl (which may be further substituted with hydroxy),
   4- to 6-membered heterocyclyl optionally substituted with 1-2 groups independently selected from halogen, oxo, and hydroxy, and
   C₁-C₆ linear and branched alkylsulfonyl;
      and R⁵ is selected from hydrogen and C₁-C₃ linear or branched alkyl.
      90. The method, compound, deuterated derivative, or pharmaceutically acceptable salt for use, or use according to Embodiment 88 or 89, wherein R⁵ is hydrogen.
      91. The method, compound, deuterated derivative, or pharmaceutically acceptable salt for use, or use according to any one of Embodiments 41-77, wherein:
   R is

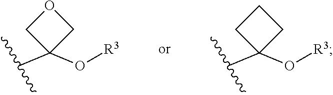

and
R³ is hydrogen.

### Non-Limiting Exemplary Embodiments 2

Without limitation, some example embodiments/clauses of this disclosure include:
   1. A compound selected from Formula I′:

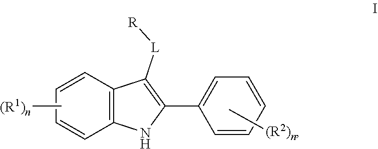

and deuterated derivatives and pharmaceutically acceptable salts thereof, wherein:
(i) R is selected from —C(O)NR³R⁴, —NR⁵C(O)R³, —NR⁵C(O)NR³R⁴, —NR^{X}—SO₂R³, —OC(O)NR³R⁴, —C(O)OR³,

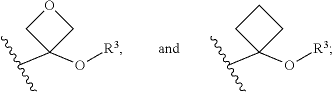

(ii) L is selected from divalent C₁-C₆ linear and branched alkyl, divalent C₂-C₆ linear and branched alkenyl, divalent C₂-C₆ linear and branched alkynyl, and divalent 1 to 6 membered heteroalkyl, wherein the divalent alkyl and divalent heteroalkyl are optionally substituted with 1-4 groups selected from:
C₁-C₆ alkyl,
aryl,
heteroaryl,
halogen,
hydroxy, and
amino;
   (iii) each R¹ is independently selected from:
halogen,
hydroxy,
thiol,
amino,
cyano,
C₁-C₆ linear, branched, and cyclic alkyl optionally substituted with 1-3 groups independently selected from hydroxy and halogen,
C₂-C₆ linear, branched, and cyclic alkenyl,
C₁-C₆ linear, branched, and cyclic alkoxy, optionally substituted with 1-3 groups independently selected from halogen,
C₁-C₆ linear, branched, and cyclic thioalkyl, optionally substituted with 1-3 groups independently selected from halogen,
C₁-C₆ linear, branched, and cyclic aminoalkyl, optionally substituted with 1-3 groups independently selected from halogen,
or two R′ groups, together with the carbon atoms to which they are attached, may form a C₄-C₈ cycloalkyl, aryl, or heteroaryl;
   (iv) each R² is independently selected from:
halogen,
hydroxy,
thiol,
amino,
cyano,
C₁-C₄ linear, branched, and cyclic alkyl optionally substituted with 1-3 groups independently selected from hydroxy and halogen,
C₂-C₄ linear, branched, and cyclic alkenyl,
C₁-C₄ linear, branched, and cyclic alkoxy, optionally substituted with 1-3 groups independently selected from halogen,
C₁-C₄ linear, branched, and cyclic thioalkyl, optionally substituted with 1-3 groups independently selected from halogen,
C₁-C₄ linear, branched, and cyclic aminoalkyl, optionally substituted with 1-3 groups independently selected from halogen,
   (v) each n is independently selected from 0, 1, 2, 3, and 4;
   (vi) R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl, and C₃-C₆ cycloalkyl,
amide optionally substituted with 1-2 groups selected from C₁-C₃ alkyl,
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl optionally substituted with 1-2 groups independently selected from:
   halogen,
   hydroxy,
   oxo,
   amino optionally substituted with 1-2 groups independently selected from hydrogen and C₁-C₆ linear or branched alkyl,
   aryl optionally substituted with 1-2 groups independently selected from halogen,
   C₁-C₆ linear and branched alkyl groups (which may be further substituted with 1-3 groups selected from hydroxy, oxo, halogen, and C₁-C₆ linear and branched alkoxy groups),
   carbamate optionally substituted with 1-2 groups independently selected from
C₁-C₆ linear and branched alkyl,
   C₁-C₆ linear and branched alkoxy, and
   amide,
4- to 10-membered heterocyclyl optionally substituted with 1-3 groups independently selected from:
   halogen,
   oxo,
   hydroxy, and
   C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, C₁-C₆ linear and branched alkyl (which may be further substituted with one or two groups selected from hydroxy and C₁-C₆ linear and branched alkoxy groups),
4- to 10-membered heteroaryl optionally substituted with 1-3 groups selected from:
   amino,
   hydroxy,
   oxo,
   halogen, and
   C₁-C₆ linear alkyl (which may be further substituted with 1-3 groups independently selected from halogen, and amino),
C₁-C₆ linear and branched alkyl groups, wherein the alkyl groups are optionally substituted with one to four groups selected from:
   amino groups optionally substituted with 1-2 groups independently selected from C₁-C₆ linear, branched, and cyclic alkyl (which may be further substituted with 1-2 oxo), and C₁-C₆ linear and branched alkylsulfonyl,
   hydroxy,
   oxo,
   cyano,
   carboxylic acid,
   sulfonic acid,
   —O-heteroaryl,
   carbamate optionally substituted with 1-2 groups independently selected from
C₁-C₆ linear and branched alkyl,
   halogen,
   amido optionally substituted with 1-2 groups independently selected from hydroxy, C₁-C₆ linear, branched, and cyclic alkyl groups and C₁-C₆ linear, branched, and cyclic hydroxyalkyl,
   C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
   C₁-C₆ linear and branched alkynyl,
   C₁-C₆ linear and branched alkoxy optionally substituted with 1-2 hydroxy,
   C₁-C₆ linear and branched alkylsulfonyl,
   aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
   carbonyl-(4-methylpiperazin-1-yl),
   carbonyl-(N-morpholino),
   4- to 10-membered heterocyclyl optionally substituted with 1-2 groups independently selected from halogen, oxo, hydroxy, and C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-3 groups independently selected from halogen, oxo, hydroxy, and C₁-C₆ linear, branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
   amino optionally substituted with 1-2 groups independently selected from hydrogen and C₁-C₆ linear, branched, and cyclic alkyl,
   halogen,
   hydroxy,
   oxo,
   C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl)
   amide optionally substituted with 1-2 groups independently selected from C₁-C₆ linear and branched alkyl,
   carbamate optionally substituted with 1-2 groups independently selected from C₁-C₆ linear and branched alkyl,
   carboxamide optionally substituted with 1-2 groups independently selected from
   C₁-C₆ linear and branched alkyl,
   C₁-C₆ linear and branched alkoxy groups optionally substituted with 1-2 groups independently selected from C₁-C₆ linear, branched, and cyclic alkyl, and heterocyclyl,
   4- to 10-membered heterocyclyl optionally substituted with 1-2 groups independently selected from halogen, hydroxy, C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups);

and

(vii) R⁵ is selected from hydrogen and linear or branched C₁-C₆ alkyl;
with the provisos that (1) the compound is not selected from

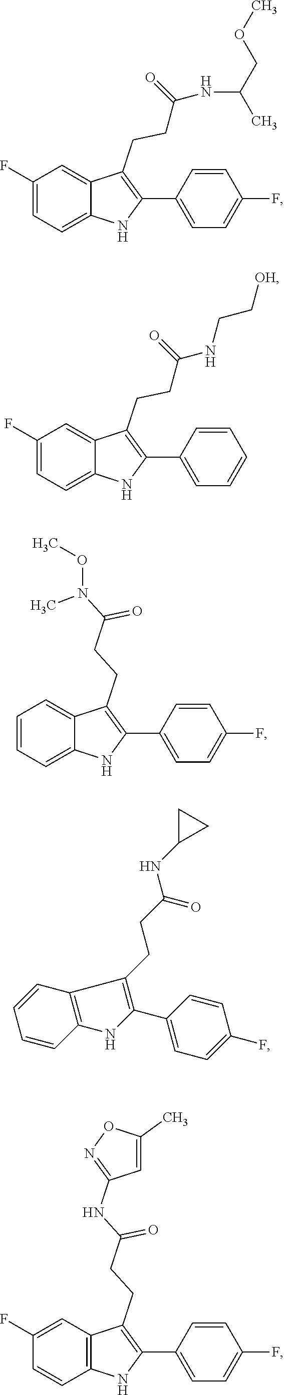 [SMILES: COCC(C)NC(=O)CCc1c(-c2ccc(F)cc2)[nH]c2ccc(F)cc12 ; O=C(CCc1c(-c2ccccc2)[nH]c2ccc(F)cc12)NCCO ; CON(C)C(=O)CCc1c(-c2ccc(F)cc2)[nH]c2ccccc12 ; O=C(CCc1c(-c2ccc(F)cc2)[nH]c2ccccc12)NC1CC1 ; Cc1cc(NC(=O)CCc2c(-c3ccc(F)cc3)[nH]c3ccc(F)cc23)no1]

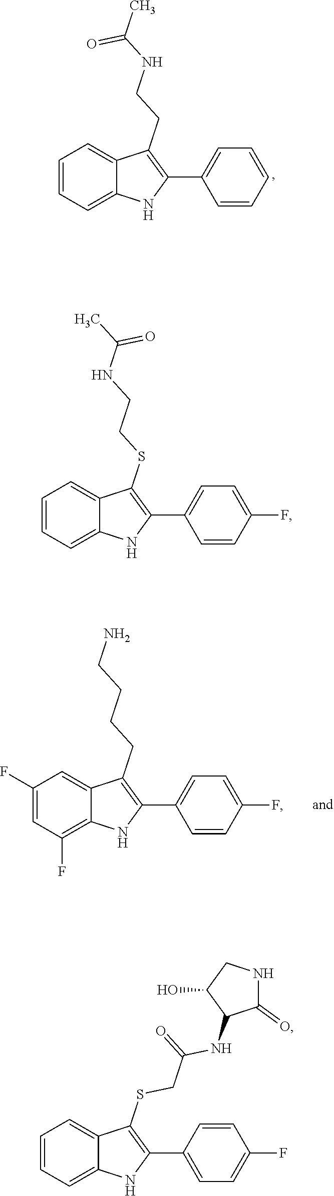

and

(2) when -L-R in Formula I′ is

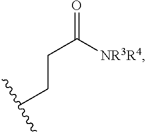

then R³ and R⁴ are not

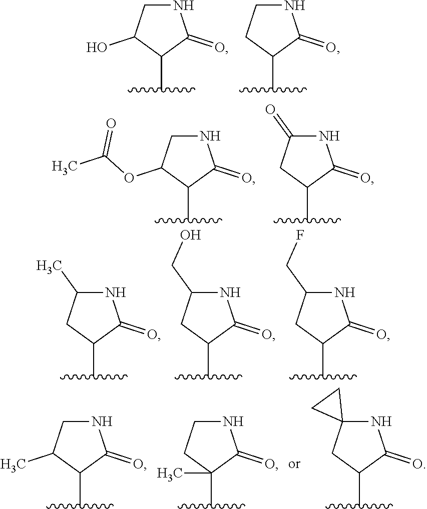

2. The compound, deuterated derivative, or pharmaceutically acceptable salt according to Clause 1, wherein each R¹ is independently selected from halogen, hydroxy, amino, C₁-C₆ linear, branched alkyl (optionally substituted with 1-3 groups independently selected from hydroxy and halogen), C₃-C₆ cycloalkyl, and C₁-C₆ linear and branched alkoxy (optionally substituted with 1-3 groups independently selected from halogen).
3. The compound, deuterated derivative, or pharmaceutically acceptable salt according to Clause 1 or Clause 2, wherein each R² is independently selected from halogen, hydroxy, amino, cyano, C₁-C₆ linear and branched alkyl (optionally substituted with 1-3 groups independently selected from hydroxy and halogen), and C₁-C₆ linear and branched alkoxy (optionally substituted with 1-3 groups independently selected from halogen).
4. The compound, deuterated derivative, or pharmaceutically acceptable salt according to any one of Clauses 1-3, wherein each R³ and/or R² are fluorine.
5. The compound, deuterated derivative, or pharmaceutically acceptable salt according to any one of Clauses 1-4, wherein each n is selected from 0, 1, and 2.
6. The compound, deuterated derivative, or pharmaceutically acceptable salt according to any one of Clauses 1-5, wherein L is selected from divalent C₁-C₆ linear and branched alkyl, and divalent C₁-C₆ linear and branched thioalkyl, wherein the divalent alkyl and divalent thioalkyl are optionally substituted with 1-2 groups independently selected from halogen.
7. The compound, deuterated derivative, or pharmaceutically acceptable salt according to Clause 6, wherein L is selected from divalent C₁-C₃ linear and branched alkyl, and divalent C₁-C₃ linear and branched thioalkyl, wherein the divalent alkyl and divalent thioalkyl are optionally substituted with 1-2 groups independently selected from halogen.
8. The compound, deuterated derivative, or pharmaceutically acceptable salt according to any one of Clauses 1-7, wherein R is —C(O)NR³R⁴, and wherein R³ and R⁴ are independently selected from:
hydrogen,
C₁-C₆ linear and branched alkylsulfonyl,
C₁-C₆ linear and branched alkoxy optionally substituted with 1-2 groups selected from hydroxy and oxo;
C₃-C₆ cyclic alkyl optionally substituted with 1-2 groups selected from:
   halogen,
   hydroxy,
   oxo,
   amino,
   aryl optionally substituted with 1-2 groups selected from halogen,
   C₁-C₆ linear and branched alkyl groups (which may be further substituted with 1-3 groups selected from hydroxy and halogen),
   carbamate optionally substituted with 1-2 groups independently selected from
C₁-C₆ linear and branched alkyl,
   C₁-C₆ linear and branched alkoxy, and
   amido groups,
4- to 10-membered heterocyclyl optionally substituted with 1-3 groups independently selected from:
   oxo,
   hydroxy,
   C₁-C₆ linear and branched alkyl (which may be further substituted with 1-2 groups independently selected from hydroxy, oxo, and C₁-C₆ linear and branched alkoxy),
4- to 10-membered heteroaryl optionally substituted with 1-3 groups selected from:
   hydroxy,
   oxo,
   halogen, and
   C₁-C₆ linear alkyl (which may be further substituted with 1-3 groups independently selected from halogen),
C₁-C₆ linear and branched alkyl groups, wherein the alkyl groups are optionally substituted with one to four groups selected from:
   amino groups optionally substituted with 1-2 groups independently selected from C₁-C₆ linear, branched, and cyclic alkyl,
   hydroxy,
   oxo,
   cyano,
   carbamate optionally substituted with 1-2 groups independently selected from
C₁-C₆ linear and branched alkyl,
   halogen,
   amido,
   C₃-C₆ cyclic alkyl optionally substituted with 1-2 hydroxy, and
   4- to 10-membered heterocyclyl optionally substituted with 1-2 independently groups independently selected from oxo, hydroxy, and C₁-C₆ linear and branched alkyl (which may be further substituted with 1-2 hydroxy), and
   4- to 10-membered heteroaryl optionally substituted with 1-3 groups independently selected from halogen and C₁-C₆ linear, branched, and cyclic alkyl (which may be further substituted with 1-3 groups independently selected from halogen).
or R³ and R⁴, together with the nitrogen atom to which they are attached, form a 4- to 10-membered heterocyclyl or heteroaryl optionally substituted with 1-3 groups independently selected from:
   amino,
   halogen,
   hydroxy,
   C₁-C₆ linear, branched, and cyclic alkyl optionally substituted with 1-2 groups independently selected from hydroxy, amino, and carbamate (which may be further substituted with 1-2 groups independently selected from C₁-C₆ linear and branched alkyl), and
   carbamate optionally substituted with 1-2 groups independently selected from C₁-C₆ linear and branched alkyl.
      9. The compound, deuterated derivative, or pharmaceutically acceptable salt according to any one of Clauses 1-7, wherein R is —NR⁵—C(O)R³, and wherein R³ is selected from:
   hydrogen,
   C₁-C₆ linear and branched alkyl optionally substituted with 1-4 groups independently selected from halogen hydroxy, cyano, amide (which may be further substituted by 1-2 groups independently selected from C₁-C₃ alkyl), amino (which may be further substituted with C₁-C₃ alkylsulfonyl), carbamate (which may be further substituted with C₁-C₆ linear and branched alkyl), 4- to 6-membered heterocyclyl (which may be further substituted with 1-2 groups independently selected from halogen, oxo, and hydroxy), 4- to 6-membered heteroaryl (which may be further substituted with 1-2 groups independently selected from halogen, oxo, hydroxy, and C₁-C₃ alkyl), and C₃-C₆ cycloalkyl (which may be further substituted with carbamate (which may be further substituted with C₁-C₆ linear or branched alkyl));
   amide optionally substituted with 1-2 groups independently selected from C₁-C₃ alkyl,
   C₁-C₆ linear and branched alkylsulfonyl,
   C₁-C₆ linear and branched alkoxy optionally substituted with 1-2 groups independently selected from hydroxy, oxo, C₃-C₆ cycloalkyl (which may be further substituted with carboxylic acid), and 3- to 6-membered heteroaryl;
   4- to 6-membered heterocyclyl optionally substituted with 1-2 groups independently selected from halogen, oxo, hydroxy, amino, and C₁-C₃ alkyl (which may be further substituted with 1-3 groups selected from halogen),
   C₃-C₆ cycloalkyl optionally substituted with 1-2 groups independently selected from amide, hydroxy, halogen, C₁-C₆ linear and branched alkyl (which may be further substituted with 1-3 groups selected from halogen), and carbamate (which may be further substituted with C₁-C₆ linear and branched alkyl), and
   4- to 6-membered heteroaryl optionally substituted with 1-2 groups independently selected from amino, halogen, oxo, hydroxy, and C₁-C₆ linear and branched alkyl (which may be further substituted with 1-3 groups selected from halogen);
      and R⁵ is selected from hydrogen and linear or branched C₁-C₃ alkyl.
      10. The compound, deuterated derivative, or pharmaceutically acceptable salt according to Clause 9, wherein R⁵ is hydrogen.
      11. The compound, deuterated derivative, or pharmaceutically acceptable salt according to any one of Clauses 1-7, wherein R is —NR³R⁴, and wherein R³ and R⁴ are independently selected from:
   4- to 6-membered heterocyclyl optionally substituted with 1-2 groups independently selected from hydroxy, halogen, and oxo,
   C₁-C₃ alkyl optionally substituted with hydroxy, oxo, or halogen, and
   hydrogen;
or R³ and R⁴, together with the nitrogen atom to which they are attached, form a 4- to 10-membered heterocyclyl optionally substituted with 1-3 groups independently selected from oxo, and C₁-C₃ alkyl.
   12. The compound, deuterated derivative, or pharmaceutically acceptable salt according to any one of Clauses 1-7, wherein R is —OR³, and wherein R³ is selected from: hydrogen, and C₁-C₆ linear and branched alkyl.
   13. The compound, deuterated derivative, or pharmaceutically acceptable salt according to any one of Clauses 1-7, wherein R is —OC(O)NR³R⁴, and wherein R³ is selected from:
   C₁-C₆ linear and branched alkyl optionally substituted with 1-4 groups independently selected from halogen, hydroxy, amide, cyano, C₃-C₆ cycloalkyl (which may be further substituted with hydroxy or C₁-C₃ alkoxy), 4 to 6 membered heteroaryl (which may be further substituted with C₁-C₃ alkyl, or trifluoro substituted C₁-C₃ alkyl), and 4- to 6-membered heterocyclyl (which may be further substituted with 1-3 groups independently selected from oxo, and hydroxy),
   C₁-C₆ linear and branched alkoxy,
   C₃-C₆ cycloalkyl optionally substituted with 1-2 groups independently selected from hydroxy, halogen, amide, C₁-C₃ alkyl (which may be further substituted with hydroxy or halogen), and C₁-C₃ alkoxy,
   4- to 6-membered heterocyclyl optionally substituted with 1-2 groups independently selected from hydroxy, halogen, oxo, and C₁-C₃ alkyl, and
   4- to 6-membered heteroaryl optionally substituted with 1-2 groups independently selected from hydroxy, halogen, and oxo,
or R³ and R⁴, together with the nitrogen atom to which they are attached, form a 4- to 10-membered heterocyclyl optionally substituted with 1-3 groups independently selected from oxo, hydroxy, and C₁-C₃ alkyl.
   14. The compound, deuterated derivative, or pharmaceutically acceptable salt according to any one of Clauses 1-7, wherein R is —NR⁵—SO₂R³, and wherein R³ is selected from:
   C₁-C₆ linear and branched alkyl optionally substituted with 1-2 groups independently selected from hydroxy, halogen, and 4- to 6-membered heterocyclyl (which may be further substituted with 1-2 groups independently selected from halogen, oxo, and hydroxy),
   4- to 6-membered heterocyclyl,
   4- to 6-membered heteroaryl optionally substituted with C₁-C₃ alkyl, and
   amino optionally substituted with 1-2 groups independently selected from C₁-C₃ alkyl.
      15. The compound, deuterated derivative, or pharmaceutically acceptable salt according to any one of Clauses 1-7, wherein R is —C(O)OR³, and wherein R³ is selected from C₁-C₃ alkyl.
      16. The compound, deuterated derivative, or pharmaceutically acceptable salt according to any one of Clauses 1-7, wherein R is —NR⁵C(O)NR³R⁴, and wherein R³ and R⁴ are independently selected from:
   C₁-C₆ linear and branched alkyl optionally substituted with 1-4 groups independently selected from halogen, hydroxy, oxo, cyano, amino (which may be further substituted with hydroxy), amido (which may be further substituted with hydroxy), sulfonic acid, aryl (optionally substituted with hydroxy), C₃-C₆ cycloalkyl (which may be further substituted 1-2 groups independently selected from hydroxy, C₁-C₃ hydroxyalkyl), and carboxylic acid,
   C₃-C₆ cycloalkyl optionally substituted with 1-2 groups independently selected from halogen, hydroxy, and C₁-C₆ linear and branched alkyl (which may be further substituted with hydroxy),
   4- to 6-membered heterocyclyl optionally substituted with 1-2 groups independently selected from halogen, oxo, and hydroxy, and
   C₁-C₆ linear and branched alkylsulfonyl;
      and R⁵ is selected from hydrogen and linear or branched C₁-C₃ alkyl.
      17. The compound, deuterated derivative, or pharmaceutically acceptable salt according to Clause 18, wherein R⁵ is hydrogen.
      18. The compound, deuterated derivative, or pharmaceutically acceptable salt according to any one of Clauses 1-7, wherein R is

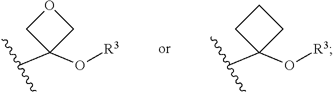

and wherein R³ is hydrogen.
19. A compound selected from Compounds 1 to 527 (Table 1), deuterated derivatives thereof, or pharmaceutically acceptable salts of any of the foregoing.
20. A pharmaceutical composition comprising the compound, deuterated derivative, or pharmaceutically acceptable salt according to any one of Clauses 1-19 and a pharmaceutically acceptable carrier.
21. A method of treating APOL1 mediated kidney disease comprising administering to a patient in need thereof a compound selected from compounds of Formula II′:

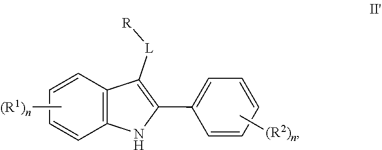

deuterated derivatives thereof, and pharmaceutically acceptable salts of any of the foregoing, wherein:
(i) R is selected from —C(O)NR³R⁴, —NR⁵C(O)R³, —NR⁵C(O)NR³R⁴, —NR³R⁴, —OR³, —NR^{X}—SO₂R³, —OC(O)NR³R⁴, —C(O)OR³

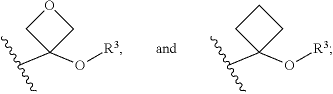

(ii) L is selected from divalent C₁-C₆ linear and branched alkyl, divalent C₂-C₆ linear and branched alkenyl, divalent C₂-C₆ linear and branched alkynyl, and divalent 1 to 6 membered heteroalkyl, wherein the divalent alkyl and divalent heteroalkyl are optionally substituted with 1-4 groups selected from:
C₁-C₆ alkyl,
aryl,
heteroaryl,
halogen,
hydroxy, and
amino;
   (iii) each R¹ is independently selected from:
halogen,
hydroxy,
thiol,
amino,
cyano,
C₁-C₆ linear, branched, and cyclic alkyl optionally substituted with 1-3 groups independently selected from hydroxy and halogen,
C₂-C₆ linear, branched, and cyclic alkenyl,
C₁-C₆ linear, branched, and cyclic alkoxy, optionally substituted with 1-3 groups independently selected from halogen,
C₁-C₆ linear, branched, and cyclic thioalkyl, optionally substituted with 1-3 groups independently selected from halogen,
C₁-C₆ linear, branched, and cyclic aminoalkyl, optionally substituted with 1-3 groups independently selected from halogen,
or two R′ groups, together with the carbon atoms to which they are attached, may form a C₄-C₈ cycloalkyl, aryl, or heteroaryl;
   (iv) each R² is independently selected from:
halogen,
hydroxy,
thiol,
amino,
cyano,
C₁-C₄ linear, branched, and cyclic alkyl optionally substituted with 1-3 groups independently selected from hydroxy and halogen,
C₂-C₄ linear, branched, and cyclic alkenyl,
C₁-C₄ linear, branched, and cyclic alkoxy, optionally substituted with 1-3 groups independently selected from halogen,
C₁-C₄ linear, branched, and cyclic thioalkyl, optionally substituted with 1-3 groups independently selected from halogen,
C₁-C₄ linear, branched, and cyclic aminoalkyl, optionally substituted with 1-3 groups independently selected from halogen,
   (v) each n is independently selected from 0, 1, 2, 3, and 4;
   (vi) R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl, and C₃-C₆ cycloalkyl,
amide optionally substituted with 1-2 groups selected from C₁-C₃ alkyl,
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl optionally substituted with 1-2 groups independently selected from:
   halogen,
   hydroxy,
   oxo,
   amino optionally substituted with 1-2 groups independently selected from hydrogen and C₁-C₆ linear or branched alkyl,
   aryl optionally substituted with 1-2 groups independently selected from halogen,
   C₁-C₆ linear and branched alkyl groups (which may be further substituted with 1-3 groups selected from hydroxy, oxo, halogen, and C₁-C₆ linear and branched alkoxy groups),
   carbamate optionally substituted with 1-2 groups independently selected from
C₁-C₆ linear and branched alkyl,
   C₁-C₆ linear and branched alkoxy, and
   amide,
4- to 10-membered heterocyclyl optionally substituted with 1-3 groups independently selected from:
   halogen,
   oxo,
   hydroxy, and
   C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, C₁-C₆ linear and branched alkyl (which may be further substituted with one or two groups selected from hydroxy and C₁-C₆ linear and branched alkoxy groups),
4- to 10-membered heteroaryl optionally substituted with 1-3 groups selected from:
   amino,
   hydroxy,
   oxo,
   halogen, and
   C₁-C₆ linear alkyl (which may be further substituted with 1-3 groups independently selected from halogen, and amino),
C₁-C₆ linear and branched alkyl groups, wherein the alkyl groups are optionally substituted with one to four groups selected from:
   amino groups optionally substituted with 1-2 groups independently selected from C₁-C₆ linear, branched, and cyclic alkyl (which may be further substituted with 1-2 oxo), and C₁-C₆ linear and branched alkylsulfonyl,
   hydroxy,
   oxo,
   cyano,
   carboxylic acid,
   sulfonic acid,
   —O-heteroaryl,
   carbamate optionally substituted with 1-2 groups independently selected from
C₁-C₆ linear and branched alkyl,
   halogen,
   amido optionally substituted with 1-2 groups independently selected from hydroxy, C₁-C₆ linear, branched, and cyclic alkyl groups and C₁-C₆ linear, branched, and cyclic hydroxyalkyl,
   C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
   C₁-C₆ linear and branched alkynyl,
   C₁-C₆ linear and branched alkoxy optionally substituted with 1-2 hydroxy,
   C₁-C₆ linear and branched alkylsulfonyl,
   aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
   carbonyl-(4-methylpiperazin-1-yl),
   carbonyl-(N-morpholino),
   4- to 10-membered heterocyclyl optionally substituted with 1-2 groups independently selected from halogen, oxo, hydroxy, and C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-3 groups independently selected from halogen, oxo, hydroxy, and C₁-C₆ linear, branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
   amino optionally substituted with 1-2 groups independently selected from hydrogen and C₁-C₆ linear, branched, and cyclic alkyl,
   halogen,
   hydroxy,
   oxo,
   C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
   amide optionally substituted with 1-2 groups independently selected from C₁-C₆ linear and branched alkyl,
   carbamate optionally substituted with 1-2 groups independently selected from C₁-C₆ linear and branched alkyl,
   carboxamide optionally substituted with 1-2 groups independently selected from
   C₁-C₆ linear and branched alkyl,
   C₁-C₆ linear and branched alkoxy groups optionally substituted with 1-2 groups independently selected from C₁-C₆ linear, branched, and cyclic alkyl, and heterocyclyl,
   4- to 10-membered heterocyclyl optionally substituted with 1-2 groups independently selected from halogen, hydroxy, C₁-C₆ linear and branched alkyl (which may be further substituted with 1-2 groups independently selected from hydroxy and C₁-C₆ linear and branched alkoxy), and
   4- to 10-membered heteroaryl optionally substituted with 1-2 groups independently selected from halogen, hydroxy, C₁-C₆ linear and branched alkyl (which may be further substituted with 1-2 groups independently selected from hydroxy and C₁-C₆ linear and branched alkoxy groups);
      and
      (vii) R⁵ is selected from hydrogen and linear or branched C₁-C₆ alkyl.
      22. The method according to Clause 21, wherein the APOL1 mediated kidney disease is chosen from ESKD, NDKD, FSGS, HIV-associated nephropathy, arterionephrosclerosis, lupus nephritis, microalbuminuria, and chronic kidney disease.
      23. The method according to Clause 21, wherein the APOL1 mediated kidney disease is FSGS.
      24. The method according to Clause 21, wherein the APOL1 mediated kidney disease is NDKD.
      25. The method according to Clause 21, wherein the APOL1 mediated kidney disease is ESKD.
      26. The method according to any one of Clauses 21-25, wherein the APOL1 mediated kidney disease is associated with APOL1 genetic alleles chosen from homozygous G1: S342G:I384M and homozygous G2: N388del:Y389del.
      27. The method according to any one of Clauses 21-25, wherein the APOL1 is associated with compound heterozygous G1: S342G:I384M and G2: N388del:Y389del APOL1 alleles.
      28. A method of inhibiting APOL1 activity comprising contacting said APOL1 with a compound selected from Formula II′, a deuterated derivative thereof, or a pharmaceutically acceptable salt of any of the foregoing.
      29. The method according to Clause 28, wherein the APOL1 is associated with APOL1 genetic alleles chosen from homozygous G1: S342G:I384M and homozygous G2: N388del:Y389del.
      30. The method according to Clause 28, wherein the APOL1 is associated with homozygous G1: S342G:I384M APOL1 alleles.
      31. The method according to Clause 28, wherein the APOL1 is associated with compound heterozygous G1: S342G:I384M and G2: N388del:Y389del APOL1 alleles.
      32. A compound selected from compounds of Formula II′, deuterated derivatives thereof, and pharmaceutically acceptable salts of any of the foregoing, for use in treating APOL1 mediated kidney disease.
      33. The compound, deuterated derivative, or pharmaceutically acceptable salt according to Clause 32, wherein the APOL1 mediated kidney disease is chosen from ESKD, NDKD, FSGS, HIV-associated nephropathy, arterionephrosclerosis, lupus nephritis, microalbuminuria, and chronic kidney disease.
      34. The method according to Clause 32, wherein the APOL1 mediated kidney disease is FSGS.
      35. The method according to Clause 32, wherein the APOL1 mediated kidney disease is NDKD.
      36. The method according to Clause 32, wherein the APOL1 mediated kidney disease is ESKD.
      37. The compound, deuterated derivative, or pharmaceutically acceptable salt according to any one of Clauses 32-36, wherein the APOL1 is associated with APOL1 genetic alleles chosen from homozygous G1: S342G:I384M and homozygous G2: N388del:Y389del.
      38. The compound, deuterated derivative, or pharmaceutically acceptable salt according to Clauses 32-36, wherein the APOL1 is associated with compound heterozygous G1: S342G:I384M and G2: N388del:Y389del APOL1 alleles.
      39. Use of a compound selected from compounds of Formula II′, deuterated derivatives thereof, and pharmaceutically acceptable salts of any of the foregoing, in the manufacture of a medicament for treating APOL1 mediated kidney disease.
      40. The use according to Clause 39, wherein the APOL1 mediated kidney disease is selected from ESKD, NDKD, FSGS, HIV-associated nephropathy, arterionephrosclerosis, lupus nephritis, microalbuminuria, and chronic kidney disease.
      41. The use according to Clause 39, wherein the APOL1 mediated kidney disease is FSGS.
      42. The use according to Clause 39, wherein the APOL1 mediated kidney disease is NDKD.
      43. The use according to Clause 39, wherein the APOL1 mediated kidney disease is ESKD.
      44. The use according to any one of Clauses 39-43, wherein the APOL1 is associated with APOL1 genetic alleles chosen from homozygous G1: S342G:I384M and homozygous G2: N388del:Y389del.
      45. The use according to any one of Clauses 39-43, wherein the APOL1 is associated with compound heterozygous G1: S342G:I384M and G2: N388del:Y389del APOL1 alleles.
      46. The method, compound for use, or use according to any one of Clauses 21-45, wherein each R¹ is independently selected from halogen, hydroxy, amino, C₁-C₆ linear, branched alkyl (optionally substituted with 1-3 groups independently selected from hydroxy and halogen), C₃-C₆ cycloalkyl, and C₁-C₆ linear and branched alkoxy (optionally substituted with 1-3 groups independently selected from halogen).
      47. The method, compound for use, or use according to any one of Clauses 21-46, wherein each R² is independently selected from halogen, hydroxy, amino, cyano, C₁-C₆ linear and branched alkyl (optionally substituted with 1-3 groups independently selected from hydroxy and halogen), and C₁-C₆ linear and branched alkoxy (optionally substituted with 1-3 groups independently selected from halogen).
      48. The method, compound for use, or use according to any one of Clauses 21-47, wherein each R¹ and/or R² are fluorine.
      49. The method, compound for use, or use according to any one of Clauses 21-48, wherein each n is selected from 0, 1, and 2.
      50. The method, compound for use, or use according to any one of Clauses 21-49, wherein L is selected from divalent C₁-C₆ linear and branched alkyl, and divalent C₁-C₆ linear and branched thioalkyl, wherein the divalent alkyl and divalent thioalkyl are optionally substituted with 1-2 groups independently selected from halogen.
      51. The method, compound for use, or use according to any one of Clauses 21-50, wherein L is selected from divalent C₁-C₃ linear and branched alkyl, and divalent C₁-C₃ linear and branched thioalkyl, wherein the divalent alkyl and divalent thioalkyl are optionally substituted with 1-2 groups independently selected from halogen.
      52. The method, compound for use, or use according to any one of Clauses 21-51, wherein R is —C(O)NR³R⁴, and wherein R³ and R⁴ are independently selected from:
   hydrogen,
   C₁-C₆ linear and branched alkylsulfonyl,
   C₁-C₆ linear and branched alkoxy optionally substituted with 1-2 groups selected from hydroxy and oxo;
   C₃-C₆ cyclic alkyl optionally substituted with 1-2 groups selected from:
      halogen,
      hydroxy,
      oxo,
      amino,
      aryl optionally substituted with 1-2 groups selected from halogen,
      C₁-C₆ linear and branched alkyl groups (which may be further substituted with 1-3 groups selected from hydroxy and halogen),
      carbamate optionally substituted with 1-2 groups independently selected from
   C₁-C₆ linear and branched alkyl,
      C₁-C₆ linear and branched alkoxy, and
      amido groups,
   4- to 10-membered heterocyclyl optionally substituted with 1-3 groups independently selected from:
      oxo,
      hydroxy,
      C₁-C₆ linear and branched alkyl (which may be further substituted with 1-2 groups independently selected from hydroxy, oxo, and C₁-C₆ linear and branched alkoxy),
   4- to 10-membered heteroaryl optionally substituted with 1-3 groups selected from:
      hydroxy,
      oxo,
      halogen, and
      C₁-C₆ linear alkyl (which may be further substituted with 1-3 groups independently selected from halogen),
   C₁-C₆ linear and branched alkyl groups, wherein the alkyl groups are optionally substituted with one to four groups selected from:
      amino groups optionally substituted with 1-2 groups independently selected from C₁-C₆ linear, branched, and cyclic alkyl,
      hydroxy,
      oxo,
      cyano,
      carbamate optionally substituted with 1-2 groups independently selected from
   C₁-C₆ linear and branched alkyl,
      halogen,
      amido,
      C₃-C₆ cyclic alkyl optionally substituted with 1-2 hydroxy, and
      4- to 10-membered heterocyclyl optionally substituted with 1-2 independently groups independently selected from oxo, hydroxy, and C₁-C₆ linear and branched alkyl (which may be further substituted with 1-2 hydroxy), and
      4- to 10-membered heteroaryl optionally substituted with 1-3 groups independently selected from halogen and C₁-C₆ linear, branched, and cyclic alkyl (which may be further substituted with 1-3 groups independently selected from halogen).
   or R³ and R⁴, together with the nitrogen atom to which they are attached, form a 4- to 10-membered heterocyclyl or heteroaryl optionally substituted with 1-3 groups independently selected from:
   amino,
   halogen,
   hydroxy,
   C₁-C₆ linear, branched, and cyclic alkyl optionally substituted with 1-2 groups independently selected from hydroxy, amino, and carbamate (which may be further substituted with 1-2 groups independently selected from C₁-C₆ linear and branched alkyl), and
   carbamate optionally substituted with 1-2 groups independently selected from C₁-C₆ linear and branched alkyl.
      53. The method, compound for use, or use according to any one of Clauses 21-52, wherein R is —NR⁵—C(O)R³, and wherein R³ is selected from:
   hydrogen,
   C₁-C₆ linear and branched alkyl optionally substituted with 1-4 groups independently selected from halogen hydroxy, cyano, amide (which may be further substituted by 1-2 groups independently selected from C₁-C₃ alkyl), amino (which may be further substituted with C₁-C₃ alkylsulfonyl), carbamate (which may be further substituted with C₁-C₆ linear and branched alkyl), 4- to 6-membered heterocyclyl (which may be further substituted with 1-2 groups independently selected from halogen, oxo, and hydroxy), 4- to 6-membered heteroaryl (which may be further substituted with 1-2 groups independently selected from halogen, oxo, hydroxy, and C₁-C₃ alkyl), and C₃-C₆ cycloalkyl (which may be further substituted with carbamate (which may be further substituted with C₁-C₆ linear or branched alkyl));
   amide optionally substituted with 1-2 groups independently selected from C₁-C₃ alkyl,
   C₁-C₆ linear and branched alkylsulfonyl,
   C₁-C₆ linear and branched alkoxy optionally substituted with 1-2 groups independently selected from hydroxy, oxo, C₃-C₆ cycloalkyl (which may be further substituted with carboxylic acid), and 3- to 6-membered heteroaryl;
   4- to 6-membered heterocyclyl optionally substituted with 1-2 groups independently selected from halogen, oxo, hydroxy, amino, and C₁-C₃ alkyl (which may be further substituted with 1-3 groups selected from halogen),
   C₃-C₆ cycloalkyl optionally substituted with 1-2 groups independently selected from amide, hydroxy, halogen, C₁-C₆ linear and branched alkyl (which may be further substituted with 1-3 groups selected from halogen), and carbamate (which may be further substituted with C₁-C₆ linear and branched alkyl), and
   4- to 6-membered heteroaryl optionally substituted with 1-2 groups independently selected from amino, halogen, oxo, hydroxy, and C₁-C₆ linear and branched alkyl (which may be further substituted with 1-3 groups selected from halogen);
      and R⁵ is selected from hydrogen and linear or branched C₁-C₃ alkyl.
      54. The method, compound for use, or use according to Clause 53, wherein R⁵ is hydrogen.
      55. The method, compound for use, or use according to any one of Clauses 21-54, wherein R is —NR³R⁴, and wherein R³ and R⁴ are independently selected from:
   4- to 6-membered heterocyclyl optionally substituted with 1-2 groups independently selected from hydroxy, halogen, and oxo,
   C₁-C₃ alkyl optionally substituted with hydroxy, oxo, or halogen, and
   hydrogen;
or R³ and R⁴, together with the nitrogen atom to which they are attached, form a 4- to 10-membered heterocyclyl optionally substituted with 1-3 groups independently selected from oxo, and C₁-C₃ alkyl.
   56. The method, compound for use, or use according to any one of Clauses 21-54, wherein R is —OR³, and wherein R³ is selected from: hydrogen, and C₁-C₆ linear and branched alkyl.
   57. The method, compound for use, or use according to any one of Clauses 21-54, wherein R is —OC(O)NR³R⁴, and wherein R³ is selected from:
   C₁-C₆ linear and branched alkyl optionally substituted with 1-4 groups independently selected from halogen, hydroxy, amide, cyano, C₃-C₆ cycloalkyl (which may be further substituted with hydroxy or C₁-C₃ alkoxy), 4 to 6 membered heteroaryl (which may be further substituted with C₁-C₃ alkyl, or trifluoro substituted C₁-C₃ alkyl), and 4- to 6-membered heterocyclyl (which may be further substituted with 1-3 groups independently selected from oxo, and hydroxy),
   C₁-C₆ linear and branched alkoxy,
   C₃-C₆ cycloalkyl optionally substituted with 1-2 groups independently selected from hydroxy, halogen, amide, C₁-C₃ alkyl (which may be further substituted with hydroxy or halogen), and C₁-C₃ alkoxy,
   4- to 6-membered heterocyclyl optionally substituted with 1-2 groups independently selected from hydroxy, halogen, oxo, and C₁-C₃ alkyl, and
   4- to 6-membered heteroaryl optionally substituted with 1-2 groups independently selected from hydroxy, halogen, and oxo,
or R³ and R⁴, together with the nitrogen atom to which they are attached, form a 4- to 10-membered heterocyclyl optionally substituted with 1-3 groups independently selected from oxo, hydroxy, and C₁-C₃ alkyl.
   58. The method, compound for use, or use according to any one of Clauses 21-54, wherein R is —NR⁵—SO₂R³, and wherein R³ is selected from:
   C₁-C₆ linear and branched alkyl optionally substituted with 1-2 groups independently selected from hydroxy, halogen, and 4- to 6-membered heterocyclyl (which may be further substituted with 1-2 groups independently selected from halogen, oxo, and hydroxy),
   4- to 6-membered heterocyclyl,
   4- to 6-membered heteroaryl optionally substituted with C₁-C₃ alkyl, and
   amino optionally substituted with 1-2 groups independently selected from C₁-C₃ alkyl.
      59. The method, compound for use, or use according to any one of Clauses 21-54, wherein R is —C(O)OR³, and wherein R³ is selected from C₁-C₃ alkyl.
      60. The method, compound for use, or use according to any one of Clauses 21-54, wherein R is —NR⁵C(O)NR³R⁴, and wherein R³ and R⁴ are independently selected from:
   C₁-C₆ linear and branched alkyl optionally substituted with 1-4 groups independently selected from halogen, hydroxy, oxo, cyano, amino (which may be further substituted with hydroxy), amido (which may be further substituted with hydroxy), sulfonic acid, aryl (optionally substituted with hydroxy), C₃-C₆ cycloalkyl (which may be further substituted 1-2 groups independently selected from hydroxy, C₁-C₃ hydroxyalkyl), and carboxylic acid,
   C₃-C₆ cycloalkyl optionally substituted with 1-2 groups independently selected from halogen, hydroxy, and C₁-C₆ linear and branched alkyl (which may be further substituted with hydroxy),
   4- to 6-membered heterocyclyl optionally substituted with 1-2 groups independently selected from halogen, oxo, and hydroxy, and
   C₁-C₆ linear and branched alkylsulfonyl;
      and R⁵ is selected from hydrogen and linear or branched C₁-C₃ alkyl.
      61. The method, compound for use, or use according to Clause 60, wherein R⁵ is hydrogen.
      62. The method, compound for use, or use according to any one of Clauses 21-54, wherein R is

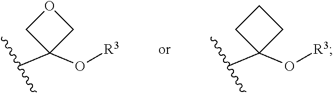

and wherein R³ is hydrogen.
63. A method of treating APOL1 mediated kidney disease comprising administering to a patient in need thereof the compound, deuterated derivative, or pharmaceutically acceptable salt according to any one of Clauses 1 to 19 or the composition according to claim 20.
64. The compound according to any one of Clauses 1 to 19, or the composition according to Clause 20 for use in treating APOL1 mediated kidney disease.
65. Use of a compound according to any one of Clauses 1 to 19 in the manufacture of a medicament for treating APOL1 mediated kidney disease.
66. The method, compound for use, or use according to any one of Clauses 63-65, wherein the APOL1 mediated kidney disease is selected from ESKD, NDKD, FSGS, HIV-associated nephropathy, sickle cell nephropathy, diabetic neuropathy, arterionephrosclerosis, lupus nephritis, microalbuminuria, and chronic kidney disease.
67. The method, compound for use, or use according to Clause 66, wherein the APOL1 mediated kidney disease is FSGS.
68. The method, compound for use, or use according to Clause 66, wherein the APOL1 mediated kidney disease is NDKD.
69. The method, compound for use, or use according to Clause 66, wherein the APOL1 mediated kidney disease is ESKD.
70. The method, compound for use, or use according to any one of Clauses 63-69, wherein the APOL1 is associated with APOL1 genetic alleles chosen from homozygous G1:
S342G:I384M and homozygous G2: N388del:Y389del.
   71. The method, compound for use, or use according to any one of Clauses 63-69, wherein the APOL1 is associated with compound heterozygous G1: S342G:I384M and G2: N388del:Y389del APOL1 alleles.

### EXAMPLES

In order that the disclosure described herein may be more fully understood, the following examples are set forth. It should be understood that these examples are for illustrative purposes only and are not to be construed as limiting this disclosure in any manner.

Throughout the synthetic schemes and descriptions for preparing compounds of Formula I, I′, II, or II′, Compounds 1 to 527, deuterated derivatives of any of those compounds, and pharmaceutically acceptable salts of any of the foregoing, the following abbreviations are used:

### Abbreviations

AIBN=Azobisisobutyronitrile
ARP=assay ready plate
BBBPY=4,4′-Di-tert-butyl-2,2′-dipyridyl
CBzCl=Benzyl chloroformate
CDMT=2-Chloro-4,6-dimethoxy-1,3,5-triazine
DIPEA=N,N-Diisopropylethylamine or N-ethyl-N-isopropyl-propan-2-amine
DMAP=dimethylamino pyridine
DMA=dimethyl acetamide
DME=dimethoxyethane
DMEM=Dulbecco's modified Eagle's medium
DMF=dimethylformamide
DMSO=dimethyl sulfoxide
DPPA=diphenylphosphoryl azide
EtOAc=Ethyl Acetate
EtOH=ethanol
FBS=fetal bovine serum
FLU=fluorescent values
HATU=[dimethylamino(triazolo[4,5-b]pyridin-3-yloxy)methylene]-dimethyl-ammonium (Phosphorus Hexafluoride Ion)
HDMC=N-[(5-Chloro-3-oxido-1H-benzotriazol-1-yl)-4-morpholinylmethylene]-N-methylmethanaminium hexafluorophosphate
HEPES=4-(2-hydroxyethyl)-1-piperazineethanesulfonic acid
HBSS=Hank's balanced salt solution
IPA=isopropyl alcohol
LDA=lithium diisopropyl amide
LED=light emitting diode
MeOH=methanol
MTBE=Methyl tert-butyl ether
NMM=N-methyl morpholine
NMP=N-methyl pyrrolidine
PBS=phosphate-buffered saline
Pd(dppf)₂Cl₂=[1,1′-Bis(diphenylphosphino)ferrocene]dichloropalladium(II)
PdCl₂(PPh₃)₂=Bis(triphenylphosphine)palladium(II) dichloride
PP=polypropylene
PTSA=p-Toluenesulfonic acid monohydrate
T3P=2,4,6-Tripropyl-1,3,5,2,4,6-trioxatriphosphorinane-2,4,6-trioxide
TEA=triethylamine
Tet=tetracycline
TFA=trifluoroacetic acid
THF=tetrahydrofuran
THP=tetrahydropyran
TMSS=Tris(trimethylsilyl)silane

### Example 1. Synthesis of Compounds

### General Schemes:

Scheme 1 refers to processes for preparation of compounds of Formula 1-6 from compounds of Formula 1-1 or 1-4. X¹ and X² are halogens such as Cl, I, or Br. Any suitable conditions for coupling an alkyne to a can be used to convert aryl halides of Formula 1-1 and alkynes of Formula 1-4 to an alkyne of Formula 1-3. For example, the coupling may be performed in the presence of a CuI and Pd(PPh₃)₂Cl₂ catalyst system. The reaction may be performed in the presence of a base (e.g. NEt₃). Conversion of compounds of Formula 1-3 to indoles of Formula 1-6 may be accomplished by treatment with CuI or PdCl₂ in a polar solvent (e.g. DMF or MeCN) in the presence of added heat (>100° C.). A compound of Formula 1-3 may also be prepared from a compound of Formula 1-4 and an aryl halide of Formula 1-5. Any suitable Sonagashira coupling condition may be used. For example, Pd(PPh₃)₂Cl₂ and CuI in the presence of a base such as DIPEA or NEt₃.

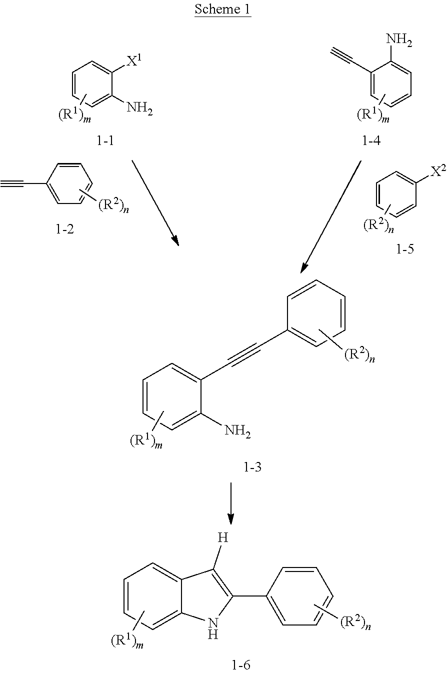

Scheme 2 refers to a process for preparation of compounds of Formula 1-6 from an indole such as that represented by Formula 2-1, and an alkyl halide of Formula 2-2, where X³ is a halogen (e.g., I or Br). R²⁰ is an alkyl group such as Me or Et. The two R²⁰ groups may be linked by a carbon carbon bond to form a cyclic boronate ester. In some embodiments, the reaction is performed in the presence of a catalyst such as PdCl₂CN₂, a ligand such as norbornylene, and a base (e.g., K₂CO₃). The reaction may be performed in a solvent such as dimethylacetamide at elevated temperature (e.g., 90° C.). Compounds of Formula 1-6 may also be prepared from indoles of Formula 2-1 and aryl boronic acids or esters of Formula 2-3. In some embodiments, the reaction is performed in the presence of a palladium catalyst (e.g., Pd(OAc)₂ trimer) in a solvent such as AcOH. The reaction is performed in the presence of oxygen.

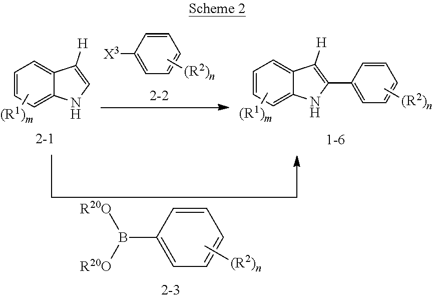

Scheme 3 refers to a process for the preparation of compounds of Formula 3-4 which may be used in the preparation of compounds of Formula 1. PG¹ is any suitable group for the protection of a carboxylic acid as an ester. For example, PG1 may be methyl, ethyl, tert-Butyl or benzyl. Any suitable conditions for a performing a Fisher indole synthesis may be used in the reaction of a ketone of Formula 3-1 with a hydrazine of Formula 3-2. For example, ZnCl₂ in a solvent such as AcOH and toluene at elevated temperature (110° C.). In an alternative embodiment, BF₃.OEt₂ in xylene solvent in the presence of added heat may be used. Any suitable conditions for the hydrolysis of an ester may be used in the preparation of compounds of Formula 3-4 from compounds of Formula 3-3.

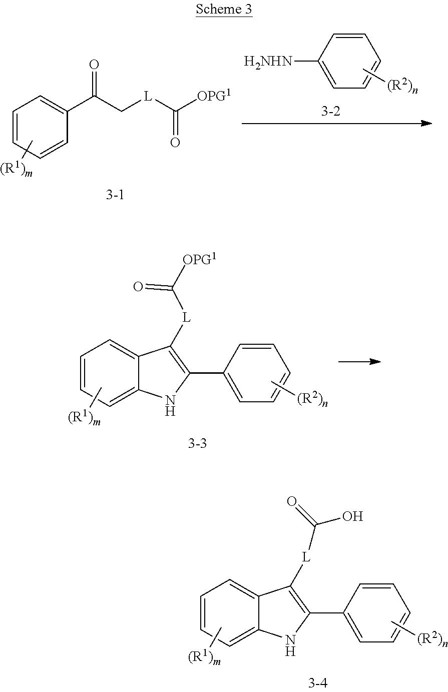

Scheme 4 refers to processes for the preparation of compounds of Formula 4-4. PG² refers to any suitable group for the protection of an amine. For example, PG² may be Boc or CBz. A compound of Formula 4-4 may be prepared from a compound of Formula 4-1 and a hydrazine of Formula 4-2 using any suitable Fisher indole synthesis conditions. In some embodiments, ZnCl₂ and AcOH may be used. The reaction may be performed in the presence of added heat. Hydrazines of Formula 4-2 may be used as free bases or as salts, such as the hydrochloride salt. A compound of Formula 4-4 may be prepared from compounds of Formula 4-3 using any suitable method for the removal of a nitrogen protecting group. For example, where PG² is a CBz group, hydrogenation conditions such as hydrogen gas in the presence of a catalyst such as palladium on carbon and a solvent such as EtOH may be used.

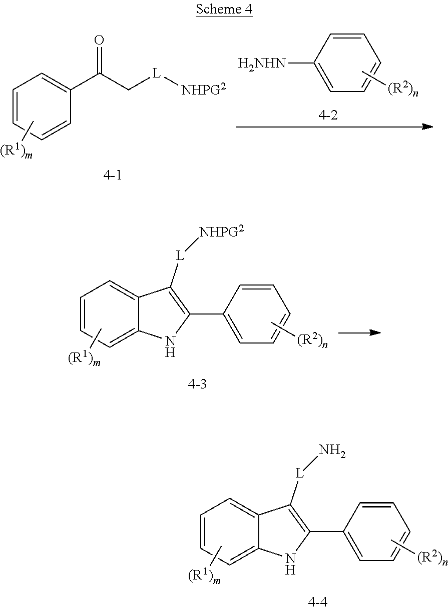

Scheme 5 refers to processes for the preparation of Formula 5-5 from compounds of Formula 2-1 by reductive alkylation reactions with acetals of Formula 5-2. In some alternative embodiments, acetals of Formula 5-2 may be substituted with their corresponding aldehydes. PG¹ is any suitable ester protecting group as defined above. L¹ is any suitable linker group which is defined within formula I. Z¹ is any suitable alkyl group (for example, ethyl or methyl). A compound of Formula 5-4 may be prepared from a compound of Formula 2-1 by reaction with an aldehyde of Formula 5-2. The reaction may be performed in the presence of an acid such as methanesulfonic acid or trifluoroacetic acid, and a reducing agent such as Et₃SiH. Any suitable conditions, such as those for the hydrolysis of an ester, may be used for converting a compound of Formula 5-4 to a compound of Formula 5-5. For example, the reaction may be performed in the presence of a base (e.g., LiOH or NaOH) in an aqueous solvent mixture (e.g., THF and water).

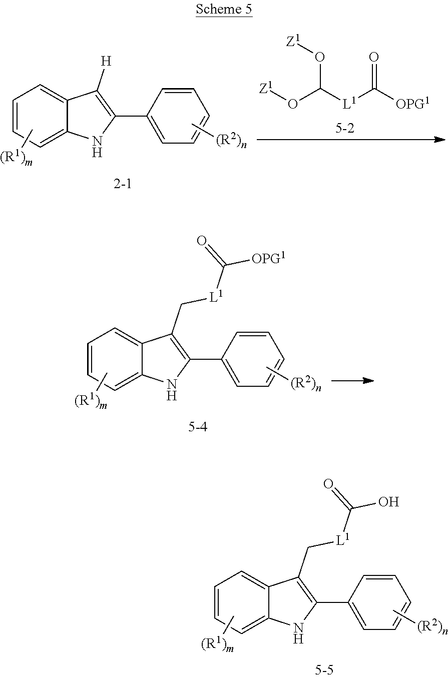

Scheme 6 shows processes for the preparation of compounds of Formula 6-4 from compounds of formula 2-1 and acetals of Formula 6-2. Z¹ is any suitable alkyl group (for example, ethyl or methyl). PG² is any suitable nitrogen protecting group. L¹ is any suitable linker group which is defined within Formula I. Any suitable conditions for reductive alkylation may be used to prepare compounds of Formula 6-3.

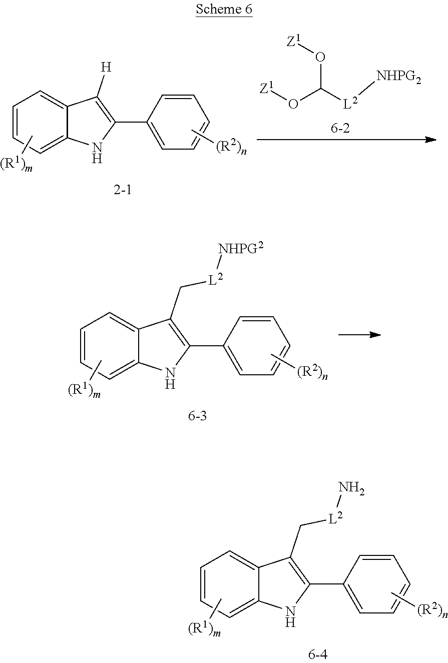

Scheme 7 shows processes for the preparation of compounds of Formula 7-2 from Formula 7-1. PG³ may be an alkyl group such as Et or Me. PG³ may also be hydrogen. L³ is any suitable linker group as described with Formula 1. Any suitable conditions for the reduction of an ester or carboxylic acid to an alcohol may be used. For example, a reducing agent such as LiAlH₄ may be used. The reduction may be performed in a solvent such as THF. The reaction may be performed at reduced temperature, for example, at 0° C.

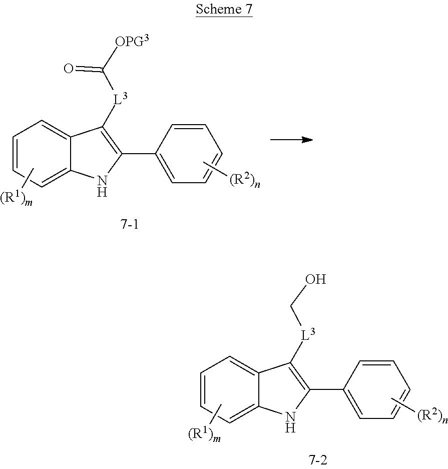

Compounds of Formula 8-1 may be prepared from compounds of Formula 2-1 using any suitable halogenating reagent (e.g., N-iodosuccinimide). Any suitable alkyne coupling reactions can be used for converting compounds of Formula 8-1 to such as those of Formula 8-3. For example, the reaction is performed in the presence of catalysts such as Pd(PPh₃)₂Cl₂ and CuI, and a base (e.g., DIPEA or TEA).

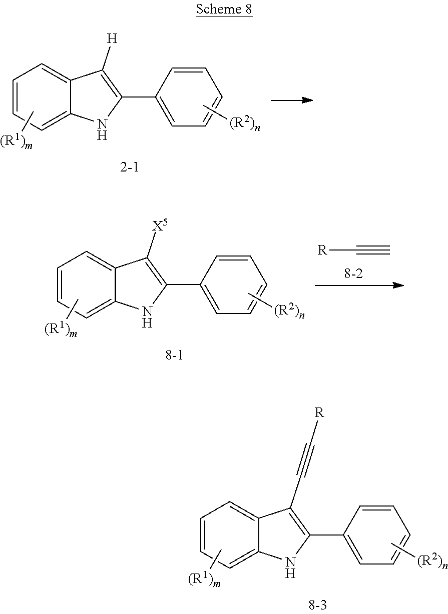

Scheme 9 depicts processes for the preparation of compounds of Formula 9-2. Any suitable conditions, such as those for the formation of an amide from a carboxylic acid can be used for reacting a compound of Formula 5-5 with an amine of Formula 9-1 to provide compounds of Formula 9-2. In some embodiments, processes for preparing compounds of Formula 9-2 comprise reacting a compound of Formula 5-5 with an amine of Formula 9-1 in the presence of an amide coupling agent (e.g., HATU, CDMT, HDMC, or T3P) and a suitable base (e.g., DIPEA or TEA), as depicted in Scheme 9. In some embodiments, at least one solvent is DMF or dichloromethane.

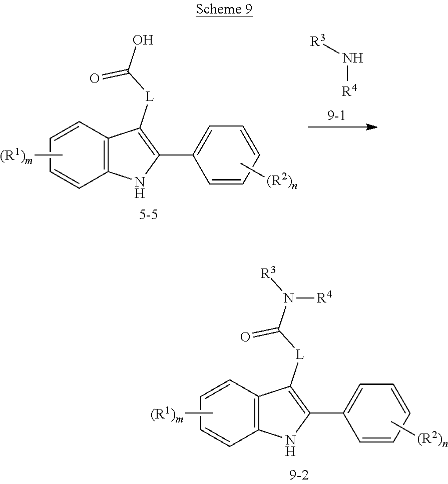

Scheme 10 shows processes for the preparation of compounds of Formula 10-2 from an amine of Formula 4-4 and a carboxylic acid of Formula 10-1. Any suitable method for performing an amide coupling may be used.

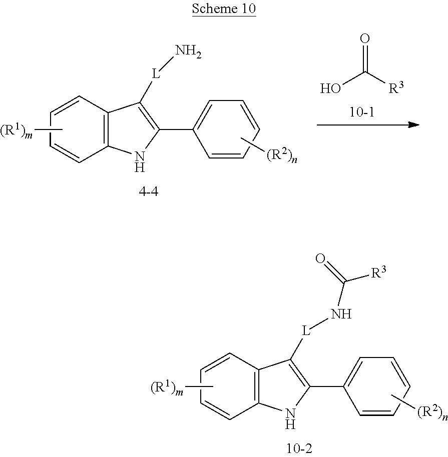

Scheme 11 describes processes for the preparation of ureas of Formula 11-3. An intermediate of Formula 11-1 where LG′ is any suitable leaving group, may be prepared from an amine of Formula 4-4. For example, LG′ may be an activated phenol group such as p-nitro phenol. Compounds of Formula 11-1 may be prepared by treatment of an amine of Formula 4-4 with a carbonate reagent such as p-nitrophenol carbonate or (4-nitrophenyl) carbonochloridate. The reaction may be performed in a basic solvent such as pyridine. In alternative conditions, compounds of Formula 11-1 may be prepared by treatment with p-nitrophenol carbonate in the presence of a base such as DIPEA, in a solvent such as DMF. Addition of an amine of Formula 11-2 to a solution of an intermediate of Formula 11-1 afforded a compound of Formula 11-3. The reaction may be performed at room temperature or with added heat.

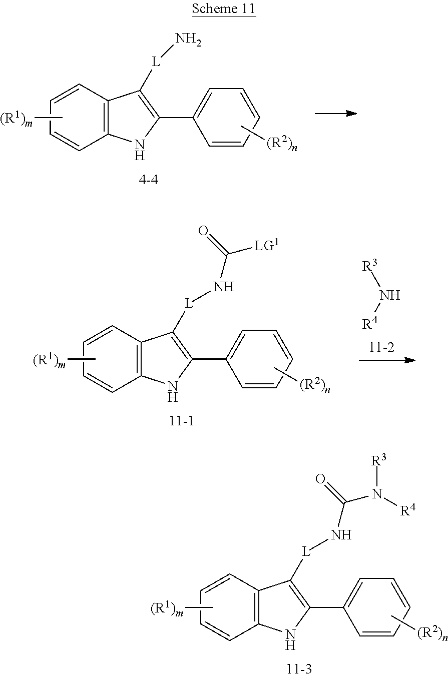

Scheme 12 shows processes for the preparation of sulfonamides of Formula 12-2. LG² represents any suitable leaving group atom or group. For example, LG² may be a chlorine atom. Reaction of an amine of Formula 4-4 with a sulfonyl reagent of Formula 12-1 in the presence of a base such as DIPEA and in a solvent such as DMF.

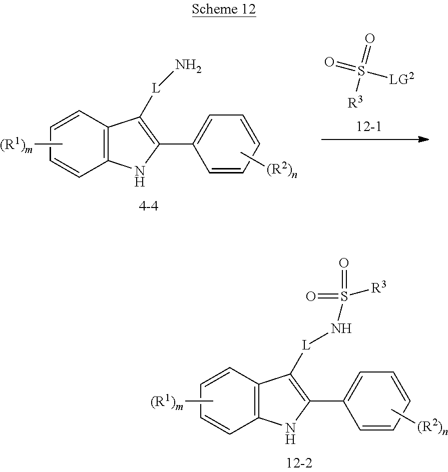

A process depicting methods for the preparation of carbamates of Formula 13-3 is shown in scheme 13. LG³ represents any suitable leaving group atom or group. For example, LG² may be a p-nitrophenol group intermediate of Formula 13-1 is prepared from alcohols of Formula 7-2 and a reagent such as (4-nitrophenyl) carbonochloridate in a solvent such as pyridine. The reaction may be performed at room temperature. A compound of Formula 13-2 may be prepared from a p-nitrophenol group intermediate of Formula 13-1 by treatment with an amine of Formula 13-2. In some embodiments, the reaction may be performed in a solvent such as DMF. A base such as pyridine may be present. The reaction may be performed in the presence of added heat (e.g., 80° C.).

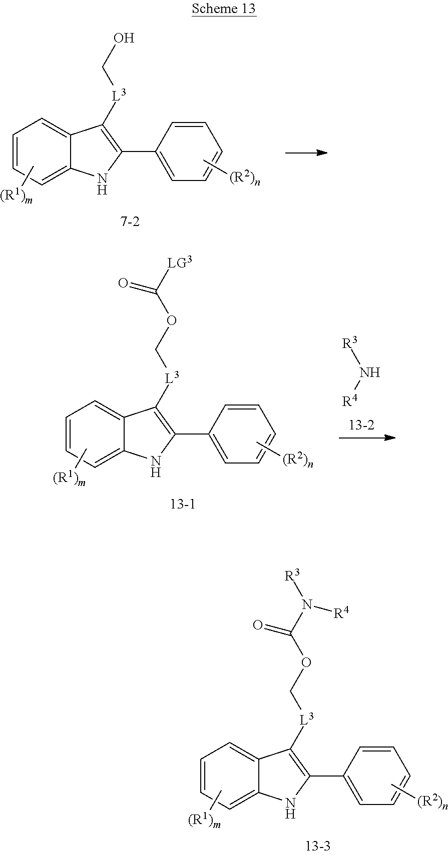

### General Purification and Analysis Methods

Unless otherwise stated, all final products were purified, as necessary, by reversed-phase HPLC (Method: C18 Waters Sunfire column (30×150 mm, 5 micron). Gradient: 10-100% MeCN in H₂O. Modifier: 0.2% formic acid or 0.1% Trifluoroacetic acid).

Products were analyzed by LCMS methods A, B, or C. LCMS m/z and retention times were collected.

LCMS Method A: HPLC Sunfire C18 column. Gradient: 2-98% MeCN/H₂O over 3.8 minutes. TFA Modifier.
LCMS Method B: UPLC CSH C18 column. Gradient: 5-95% MeCN/H₂O. TFA Modifier.
LCMS Method C: UPLC CSH C18 column. Gradient: 10-60% MeCN/H₂O. TFA Modifier.

### Preparation S1

### (3S,4R)-3-amino-4-hydroxy-pyrrolidin-2-one (S1)

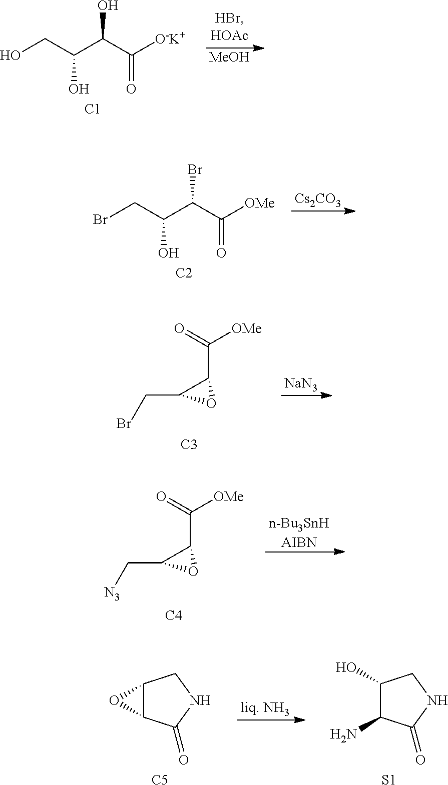

### Step 1. Synthesis of methyl (2S,3R)-2,4-dibromo-3-hydroxy-butanoate (C2)

Potassium (2R,3R)-2,3,4-trihydroxybutanoate C1 (10 g, 57.1 mmol) was stirred with HBr in acetic acid (154 g, 103 mL of 30% w/w, 570.8 mmol) for 16 hours. Anhydrous MeOH (250 mL) was added and the mixture heated at reflux for 4 hours. The mixture was concentrated to dryness and the residue dissolved in EtOAc (100 mL). The solution was washed with water (50 mL) and brine (50 mL), then dried over Na₂SO₄, and concentrated in vacuo. Purification by silica gel chromatography (Gradient: 15-20% EtOAc in hexane) afforded the product as a colorless liquid. Methyl (2S,3R)-2,4-dibromo-3-hydroxy-butanoate (13 g, 83%). ¹H NMR (400 MHz, Chloroform-d) δ 4.71 (d, J=3.4 Hz, 1H), 4.17-4.14 (m, 1H), 3.82 (s, 3H), 3.53-3.44 (m, 2H).

### Step 1. Alternative Procedure for Synthesis of methyl (2S,3R)-2,4-dibromo-3-hydroxy-butanoate (C2)

Potassium (2R,3R)-2,3,4-trihydroxybutanoate C1 (280 g) was stirred with a 33% solution of HBr in acetic acid (1 L) at room temperature for 24 hours. The reaction mixture was then poured into MeOH (5 L). The mixture was stirred at room temperature for 8 hours, then at 65° C. for 4 hours. The mixture was concentrated, the residue was dissolved in MeOH (1.2 L) and then concentrated sulfuric acid (30 mL) was slowly added. The mixture was heated under reflux for 6 hours, then concentrated. The residue was taken up with EtOAc (400 mL). The resulting solution was washed with water (250 mL), dried over Na₂SO₄, filtered and concentrated in vacuo to give the product as an oil which solidified upon storage at 4° C. (375 g, 74%).

### Step 2. Synthesis of methyl (2R,3S)-3-(bromomethyl)oxirane-2-carboxylate (C3)

Methyl (2R,3R)-2,4-dibromo-3-hydroxy-butanoate C2 (524.8 g, 1.9 mol) was dissolved in acetone (4.5 L) in a 12 L round-bottomed flask equipped with an overhead stirrer. The reaction was cooled to 0° C. in an ice-bath and Cs₂CO₃ (994 g, 3.1 mol) was added. The reaction was stirred for 30 minutes at 0° C. and then for 2 hours at room temperature. The mixture was filtered, washed with acetone, and then concentrated in vacuo to afford a dark gray oil residue. The product was dissolved in CH₂Cl₂ and filtered over a short plug of silica gel, eluting with CH₂Cl₂ (approximately 1 L). The filtrate was concentrated in vacuo to afford the product as a clear yellow oil (377.3 g, quantitative). ¹H NMR (300 MHz, Chloroform-d) δ 3.83 (s, 3H), 3.71-3.61 (m, 2H), 3.61-3.53 (m, 1H), 3.46 (dd, J=9.9, 6.6 Hz, 1H). ¹³C NMR (75 MHz, Chloroform-d) δ 167.58, 55.89, 53.52, 52.77, 26.83.

### Step 2. Alternative Procedure for Synthesis of methyl (2R,3S)-3-(bromomethyl)oxirane-2-carboxylate (C3)

To a solution of methyl (2R,3R)-2,4-dibromo-3-hydroxy-butanoate C₂ (200 g, 0.73 mol) in acetone (2.0 L) was added anhydrous K₂CO₃ (151.1 g, 1.1 mol), while the reaction temperature was maintained at 0-5° C. The reaction was stirred at 0-5° C. for 2 hours, then gradually warmed to room temperature over 4 hours The reaction mixture was filtered, and the filtrate was concentrated under reduced pressure. The residue was distilled under vacuum 75-80° C./200-300 Pa to give the product as a colorless liquid (105 g, 74%).

### Step 3. Synthesis of methyl (2R,3R)-3-(azidomethyl)oxirane-2-carboxylate (C4)

Methyl (2R,3S)-3-(bromomethyl)oxirane-2-carboxylate C3 (52.6 g, 269.7 mmol) was dissolved in DMF (500 mL) in a 3 L round-bottomed flask equipped with a magnetic stir bar. NaN₃ (25.3 g, 388.4 mmol) was added and the mixture was stirred at room temperature for 1 hour. The reaction was poured into water, and extracted with EtOAc. The extract was washed with water, dried over MgSO4, and concentrated in vacuo to afford a dark red oil. The oil residue was dissolved in CH₂Cl₂, and filtered over a plug of silica gel eluting with CH₂Cl₂. The filtrate was concentrated in vacuo to afford the product, C4, as a clear, light red oil (40.8 g, 96%). ¹H NMR (300 MHz, Chloroform-d) δ 3.87-3.74 (m, 3H), 3.67-3.55 (m, 2H), 3.47 (dd, J=13.3, 5.1 Hz, 1H), 3.38 (ddd, J=6.3, 5.0, 4.4 Hz, 1H). ¹³C NMR (75 MHz, Chloroform-d) δ 167.76, 54.81, 52.67, 51.32, 48.74.

### Step 4. Synthesis of (1R,5R)-6-oxa-3-azabicyclo[3.1.0]hexan-2-one (C5)

A 2 L 3-neck flask with overhead stirrer was charged with methyl (2R,3R)-3-(azidomethyl)oxirane-2-carboxylate C4 (67 g, 402.5 mmol) in toluene (500 mL), stirred for 10 minutes, and then warmed to 80° C. Bu₃SnH (220 mL, 817.8 mmol) and AIBN (2 g, 12.2 mmol) were dissolved in toluene (500 mL) and then added to the reaction over 3 hours using an additional funnel. The resulting reaction mixture was stirred at 80-87° C. for 1 hour, then cooled to ambient temperature, and concentrated under reduced pressure. The residue was partitioned between acetonitrile (2 L) and pentane (1 L), stirred for 10 minutes and then the acetonitrile phase (bottom) was separated. The acetonitrile phase was washed with pentane (2×500 mL) and concentrated in vacuo to afford a light yellow solid. The solid residue was triturated with pentane (˜200 mL) to afford the product as a yellow solid which was used without further purification (52 g, 98%). ¹H NMR (300 MHz, Chloroform-d) δ 5.89 (s, 1H), 4.00 (q, J=2.5 Hz, 1H), 3.74-3.50 (m, 2H), 3.44 (dd, J=12.4, 2.4 Hz, 1H). ¹³C NMR (75 MHz, Chloroform-d) δ 173.24, 53.28, 52.18, 44.00.

### Step 5. Synthesis of (3S,4R)-3-amino-4-hydroxy-pyrrolidin-2-one (S1)

A Parr vessel containing (1R,5R)-6-oxa-3-azabicyclo[3.1.0]hexan-2-one C5 (60 g, 605.5 mmol) and NH₃ (1.5 L, 58.6 mol) was pressurized to 200 psi and allowed to stir at 18° C. for 2 days. NH₃ was released from the vessel to provide a grey solid. Heptane was added and the mixture stirred for 30 minutes. The solid was filtered, and then the filter cake was isolated, and then EtOAc and heptane to the solid. The mixture was concentrated in vacuo to afford the product (55 g, 78%). ¹H NMR (300 MHz, Water-d₂) δ 4.13 (q, J=7.2 Hz, 1H), 3.53 (dd, J=10.4, 7.4 Hz, 1H), 3.36 (d, J=7.5 Hz, 1H), 3.05 (dd, J=10.4, 6.8 Hz, 1H).

### Alternative Preparation S1

### (3S,4R)-3-amino-4-hydroxypyrrolidin-2-one hydrochloride (S1)

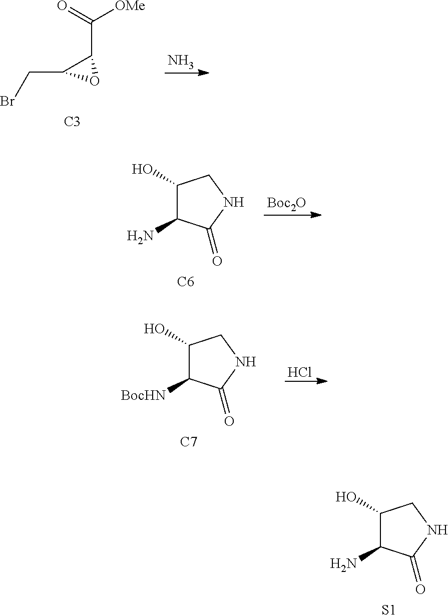

### Steps 1 & 2. Synthesis of N-Boc-(3S,4R)-3-amino-4-hydroxypyrrolidin-2-one (C7)

At −60° C., ammonia gas was condensed into an autoclave containing a frozen solution of methyl (2R,3S)-3-(bromomethyl)oxirane-2-carboxylate C3 (81 g, 0.42 mol) in 1,4-dioxane (160 mL) until approx. 400 mL of liquid was collected. The autoclave was closed, allowed to warm gradually to room temperature and then heated at 50-60° C. for 2 hours. The autoclave was then cooled back to −60° C. and depressurized. The reaction mixture was warmed gradually to allow the liquid ammonia to evaporate, leaving a viscous residue. The residue was taken up with MeOH (500 mL) and the suspension was treated with a 28% solution of sodium methoxide in MeOH (86 g, 0.42 mol). The mixture was stirred at room temperature for 30 minutes then concentrated. The residue was dissolved in water (500 mL), then Na₂CO₃ (89 g, 0.84 mol) and a solution of Boc₂O (110 g, 0.5 mol) in THF (200 mL) was added. The mixture was stirred at room temperature for 10 hours. The aqueous phase was then saturated with NaCl solution and extracted with THF (3×200 mL). The combined organic phases were dried over Na₂SO₄ and concentrated in vacuo. The residue was triturated with warm MTBE (200 mL) and the precipitated solid was collected by filtration, washed with MTBE and dried under vacuum to afford the product as a white solid (28 g, 31%).

### Step 3. Synthesis of (3S,4R)-3-amino-4-hydroxypyrrolidin-2-one hydrochloride (S1)

To solution of N-Boc-(3S,4R)-3-amino-4-hydroxypyrrolidin-2-one C7 (28 g, 129 mmol) in EtOH (300 mL) heated at 50-60° C. was added a solution of HCl in EtOH (5.0 M, 75 mL). The reaction mixture was kept at 50-60° C. for 2 hours. The suspension was cooled to room temperature and the solid was collected by filtration, washed with EtOH and dried in vacuo to afford the product as an off-white solid (18 g, 90%). ¹H NMR (500 MHz, DMSO-d₆) δ 8.73 (br, 3H), 8.28 (s, 1H), 6.03 (s, 1H), 4.42-4.37 (m, 1H), 3.74 (d, J=6.8 Hz, 1H), 3.48-3.39 (m, 1H), 3.03-3.00 (m, 1H).

### Preparation S2

### (3R)-3-aminopyrrolidin-2-one (S2)

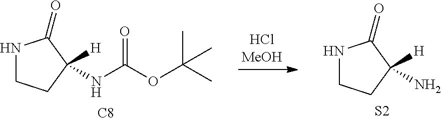

### Preparation of (3R)-3-aminopyrrolidin-2-one (S2)

To a solution of tert-butyl N-[(3R)-2-oxopyrrolidin-3-yl]carbamate C8 (458.6 mg, 2.290 mmol) in methanol (4 mL) was added HCl (2 mL of 4 M, 8.000 mmol) in 1,4-dioxane. Reaction was stirred at 50° C. for 30 minutes. Solvent was evaporated and crude product was washed with ether to afford crude (3R)-3-aminopyrrolidin-2-one (S2) (Hydrochloride salt) (350 mg, quantitative). LCMS m/z 130.05 [M+H]⁺.

### Preparation S3

### (3R)-3-aminopyrrolidine-2,5-dione (S3)

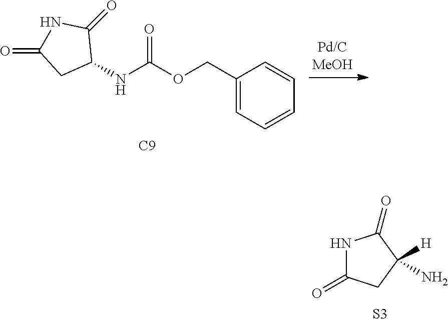

### Preparation of (3R)-3-aminopyrrolidine-2,5-dione (S3)

To a suspension benzyl N-[(3R)-2,5-dioxopyrrolidin-3-yl]carbamate C9 (862 mg, 3.47 mmol) in MeOH (10 mL) and EtOAc (5 mL) was added 5% palladium on carbon catalyst (20 mg). The mixture was subjected to hydrogenation conditions of 50 psi H2 for 2 hours. Filtration through a pad of Celite®, washing with MeOH and CH₂Cl₂, then concentration of the filtrate in vacuo afforded the product (398 mg, 98%). ¹H NMR (300 MHz, Methanol-d₄) δ 3.86 (dd, J=8.8, 5.5 Hz, 1H), 2.98 (dd, J=17.9, 8.8 Hz, 1H), 2.43 (dd, J=17.9, 5.5 Hz, 1H). LCMS m/z 123.74 [M+H]⁺.

### Preparation S4

### (1-aminocyclopropyl)methanol (S4)

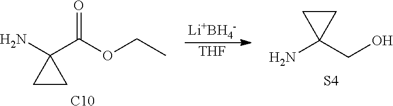

### Preparation of (1-aminocyclopropyl)methanol (S4)

To a suspension of ethyl 1-aminocyclopropanecarboxylate C10 (Hydrochloride salt) (3.7 g, 22 mmol) in THF (100 mL) was added lithium boranuide (1 g, 45.91 mmol). Reaction bubbled immediately after addition. Reaction was stirred for 24 hours. MeOH (10 mL) was added. Solvent was removed in vacuo. MeOH (10 mL) was added and the solvent was again removed in in vacuo to give (1-aminocyclopropyl)methanol (S4) (720 mg, 37%). ¹H NMR (300 MHz, Chloroform-d) δ 3.74 (s, 1H), 1.32-1.05 (m, 2H), 0.92-0.55 (m, 1H).

### Preparation S5

### [1-(3,3-difluorocyclobutyl)pyrazol-4-yl]methanamine (S5)

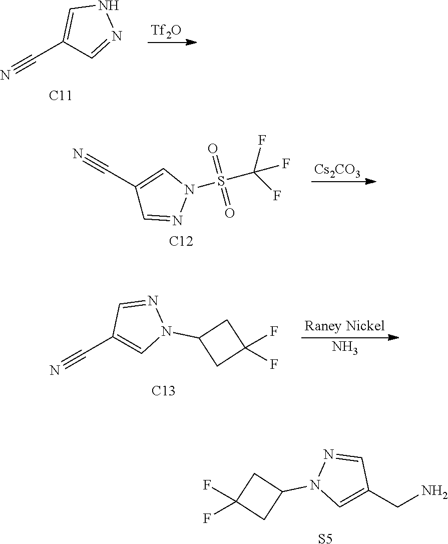

### Step 1. Synthesis of 1-(trifluoromethylsulfonyl)pyrazole-4-carbonitrile (C12)

To a solution of 1H-pyrazole-4-carbonitrile C11 (10 g, 107.4 mmol) and pyridine (26 mL, 321.5 mmol) in DCM (350 mL) was added Tf₂O (25 mL, 148.6 mmol) dropwise. The reaction was stirred at 30° C. Reaction turned orange. After complete addition the reaction was stirred for 3 hours. After 24 hours Tf₂O (10 mL, 59.44 mmol) was added. Reaction was stirred for 72 hours. H₂O (2 L) was added and aqueous layer was extracted with DCM (100 mL). Organic layer was washed with 1 M HCl (500 mL), extracted with DCM (50 mL) and combined with other organics. Combined organic extracts were dried over MgSO4, filtered, and concentrated to give 1-(trifluoromethylsulfonyl)pyrazole-4-carbonitrile C12 (20.54 g, 85%). ¹H NMR (400 MHz, Chloroform-d) δ 8.54 (d, J=0.6 Hz, 1H), 8.20 (d, J=0.5 Hz, 1H).

### Step 2. Synthesis of 1-(3,3-difluorocyclobutyl)pyrazole-4-carbonitrile (C13)

To a solution of 3,3-difluorocyclobutanol (1.06 g, 9.81 mmol) in MeCN (10 mL) was added Cs₂CO₃ (3.5 g, 10.74 mmol). Reaction was cooled to 0° C. A solution of 1-(trifluoromethylsulfonyl)pyrazole-4-carbonitrile C12 (2 g, 8.883 mmol) in MeCN (10 mL) was slowly added to the reaction mixture keeping the temperature below 30° C. Reaction was warmed to room temperature and stirred for 30 minutes. Solids were filtered off and solvent was removed under reduced pressure. Water (30 mL) and DCM (30 mL) were added. The organic layer was separated and washed with brine, dried over Na₂SO₄, filtered, and the solvent was removed in vacuo. Silica gel chromatography (Gradient: 0-100% EtOAc in heptane) afforded the product 1-(3,3-difluorocyclobutyl)pyrazole-4-carbonitrile C13 (1.38 g, 81%). ¹H NMR (400 MHz, Chloroform-d) δ 7.90 (d, J=4.3 Hz, 2H), 4.88-4.66 (m, 1H), 3.42-3.08 (m, 4H).

### Step 3. Synthesis of [1-(3,3-difluorocyclobutyl)pyrazol-4-yl]methanamine (S5)

To a solution of 1-(3,3-difluorocyclobutyl)pyrazole-4-carbonitrile C13 (2.02 g, 11.03 mmol) in MeOH (80 mL) was added Raney nickel (approximately 187.1 mg, 21.02 μL, 3.187 mmol) and ammonia (100 mL of 7 M, 700.0 mmol). The mixture was subjected to hydrogenation conditions of 50 psi H2 for 2 hours. Filtration through a pad of Celite®, washing with MeOH, then concentration of the filtrate in vacuo afforded [1-(3,3-difluorocyclobutyl)pyrazol-4-yl]methanamine (S5) (2.0405 g, 99%). ¹H NMR (400 MHz, Chloroform-d) δ 7.53 (d, J=7.2 Hz, 1H), 7.41 (s, 1H), 4.74-4.59 (m, 1H), 3.80 (s, 2H), 3.35-2.98 (m, 4H).

### Preparation S6

### (2S)-2-amino-3,3-difluoro-propan-1-ol (S6)

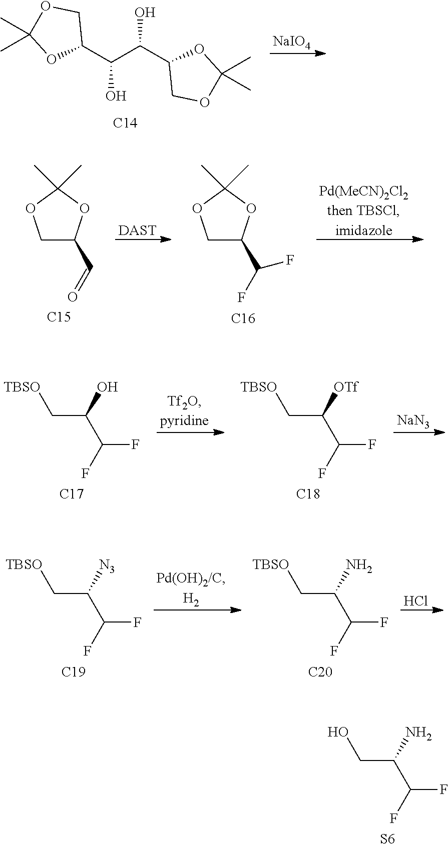

### Step 1. Synthesis of (4R)-2,2-dimethyl-1,3-dioxolane-4-carbaldehyde (C15)

To a solution of (1S,2S)-1,2-bis[(4R)-2,2-dimethyl-1,3-dioxolan-4-yl]ethane-1,2-diol C14 (30 g, 114.4 mmol) in DCM (300 mL) was added NaHCO₃ (12 mL) and the mixture was cooled to 10° C. before NaIO₄ (49 g, 229.1 mmol) was added portion wise at such a rate that internal temperature stayed below 5° C. The reaction was stirred at room temperature for 2 hours, then was filtered on Celite®. The cake was washed with DCM and the filtrate was concentrated in vacuo, yielding (4R)-2,2-dimethyl-1,3-dioxolane-4-carbaldehyde (28.0 g, 94%) ¹H NMR (400 MHz, DMSO-d₆) δ 9.60 (s, 1H), 4.52 (ddd, J=7.3, 4.6, 1.6 Hz, 1H), 4.13-4.05 (m, 2H), 1.38 (s, 3H), 1.33 (s, 3H).

### Step 2. Synthesis of (4R)-4-(difluoromethyl)-2,2-dimethyl-1,3-dioxolane (C16)

To a solution of (4R)-2,2-dimethyl-1,3-dioxolane-4-carbaldehyde C15 (9.9 g, 76 mmol) (prepared freshly) in DCM (90 mL) was added DAST (14.714 g, 12.061 mL, 91.285 mmol) at 0° C. After the addition, the solution was stirred at room temperature for 3 hours. The reaction was quenched by addition of 15% sodium bicarbonate aqueous solution. The organic layer was separated, and the aqueous layer was extracted with DCM (3×30 mL). The combined organic extracts were dried over magnesium sulfate, filtered, and the solvent carefully removed in vacuo to give crude product (4R)-4-(difluoromethyl)-2,2-dimethyl-1,3-dioxolane (12 g, quantitative). ¹H NMR (400 MHz, Chloroform-d) δ 5.81-5.53 (m, 1H) 4.28-4.18 (m, 1H), 4.13-4.01 (m, 2H), 1.45 (s, 3H), 1.37 (s, 3H). The crude material was carried to the next step without further purification.

### Step 3. Synthesis of (2R)-3-[tert-butyl(dimethyl)silyl]oxy-1,1-difluoro-propan-2-ol (C17)

To a stirred solution of (4R)-4-(difluoromethyl)-2,2-dimethyl-1,3-dioxolane C16 (11.57 g, 76.049 mmol) in MeCN (95 mL) and H₂O (5 mL), was added Pd(MeCN)₂Cl₂ (440.33 mg, 1.5210 mmol). Then the reaction mixture was heated at 60° C. for 5 hours. The reaction mixture was cooled to room temperature, filtered and the solvent was evaporated under reduced pressure. The crude product was dissolved in DCM (200 mL), followed by addition of imidazole (6.2127 g, 91.259 mmol). To the resulting solution was added dropwise a solution of TBSCl (12.608 g, 83.654 mmol) in DCM at 0° C. After addition the ice-bath was removed, and the solution was stirred at room temperature overnight. The resulting solution was diluted with DCM (60 mL) and water (60 mL). The organic layer was separated, and the water layer was extracted with DCM (2×30 mL). The combined organic layer was washed with water (2×30 mL) and brine (40 mL), dried over sodium sulfate, filtered and the solvent was removed in vacuo. Silica gel chromatography (Gradient: 5-10% EtOAc in heptane) afforded the product (2R)-3-[tert-butyl(dimethyl)silyl]oxy]-1,1-difluoro-propan-2-ol (8.6 g, 50%). ¹H NMR (400 MHz, Chloroform-d) δ 5.29 (s, 1H), 3.83-3.70 (m, 2H), 2.58 (d, J=6.3 Hz, 1H), 1.25 (s, 1H), 0.90 (d, J=3.4 Hz, 10H), 0.09 (d, J=1.4 Hz, 6H).

### Step 4. Synthesis of [(1R)-1-[[tert-butyl(dimethyl)silyl]oxymethyl]-2,2-difluoro-ethyl]trifluoromethanesulfonate (C18)

To a stirred solution of (2R)-3-[tert-butyl(dimethyl)silyl]oxy-1,1-difluoro-propan-2-ol C17 (8.6 g, 37.997 mmol) in DCM (150 mL) was added pyridine (5.4100 g, 5.5317 mL, 68.395 mmol) and trifluoromethanesulfonic anhydride (13.937 g, 8.1982 mL, 49.396 mmol) in DCM (50 mL) dropwise at −20° C. The resulting mixture was stirred at −10° C., and then stirred at 0° C. for 2 hours. To the reaction mixture was added water (200 mL) and the aqueous layer was extracted with DCM (2×200 mL). The combined organic phase was washed with water and brine, then dried over Na₂SO₄ and evaporated under reduced pressure to get the crude product [(1R)-1-[[tert-butyl(dimethyl)silyl]oxymethyl]-2,2-difluoro-ethyl]trifluoromethanesulfonate (9 g, 66%). ¹H NMR (400 MHz, Chloroform-d) δ 6.00 (td, J=4.32, 54.68 Hz, 2H), 4.88-4.82 (m, 1H), 4.12-4.10 (m, 1H), 3.98 (d, J=4.2 Hz), 0.85 (s, 9H), 0.13 (s, 6H).

### Step 5. Synthesis of [(2S)-2-azido-3,3-difluoro-propoxy]-tert-butyl-dimethyl-silane (C19)

To a stirred solution of [(1R)-1-[[tert-butyl(dimethyl)silyl]oxymethyl]-2,2-difluoro-ethyl] trifluoromethanesulfonate; methane C18 (9 g, 24.036 mmol) in DMF (100 mL) was added NaN₃ (4.6877 g, 14.107 mL, 72.108 mmol) and stirred at room temperature for 2 hours. The reaction mixture was then diluted with water (500 mL), and extracted with DCM (2×200 mL). Combined organic layer was washed with water (3×500 mL), and brine and dried over Na₂SO₄ to provide crude desired compound [(2S)-2-azido-3,3-difluoro-propoxy]-cert-butyl-dimethyl-silane (5 g, 83%). ¹H NMR (400 MHz, Chloroform-d) δ 5.82 (td, J=4.4, 55.28 Hz, 1H), 3.86-3.84 (m, 2H), 3.62-3.60 (m, 1H), 0.89 (s, 9H), 0.05 (s, 6H).

### Step 6. Synthesis of (2S)-3-[tert-butyl(dimethyl)silyl]oxy-1,1-difluoro-propan-2-amine (C20)

To a stirred solution of [(2S)-2-azido-3,3-difluoro-propoxy]-cert-butyl-dimethyl-silane C19 (5 g, 19.893 mmol) in methanol (100 mL) under argon atmosphere, was added Pd(OH)₂/C (3.4 g, 20% w/w, 4.8421 mmol). The reaction was then hydrogenated under a H2 balloon atmosphere for 2 hours. The mixture was filtered through Celite® and washed with methanol. The combined organic layer was evaporated to dryness to afford the crude compound. Purification by silica gel chromatography (Gradient: 0-30% EtOAc in heptane) yielded the product (2S)-3-[tert-butyl(dimethyl)silyl]oxy-1,1-difluoro-propan-2-amine (2.6 g, 58%). ¹H NMR (400 MHz, Methanol-d₄) δ 5.73 (td, J=4.4, 55.28 Hz, 1H), 3.76-3.65 (m, 2H), 3.04-2.99 (m, 1H), 0.88 (s, 9H), 0.06 (s, 6H).

### Step 7. Synthesis of (2S)-2-amino-3,3-difluoro-propan-1-ol (S6)

To a stirred solution of (2S)-3-[tert-butyl(dimethyl)silyl]oxy-1,1-difluoro-propan-2-amine C20 (400 mg, 1.7750 mmol) in MeOH (2 mL), was added HCl in dioxane (2.2188 mL of 4 M, 8.8750 mmol) at 0° C. and stirred at room temperature for 5 hours. The reaction mixture was evaporated under reduced pressure and the crude product obtained was triturated with diethyl ether (2×5 mL) and then dried properly to get the desired compound (2S)-2-amino-3,3-difluoro-propan-1-ol (hydrochloride salt) (230 mg, 88%). ¹H NMR (400 MHz, DMSO-d₆) δ 8.56 (s, 3H), 6.28 (td, J=3.48, 51.08 Hz, 1H), 5.56 (bs, 1H), 3.70-3.58 (m, 3H).

### Preparation S7

3-[5-fluoro-2-(4-fluorophenyl)-1H-indol-3-yl]propanoic acid (S7) was obtained from commercial sources. S7 may be prepared using analogous method to that described for S8.

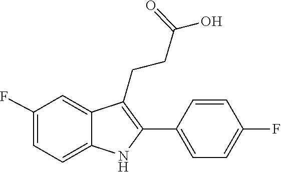 [SMILES: O=C(O)CCc1c(-c2ccc(F)cc2)[nH]c2ccc(F)cc12]

### Preparation S8

### (3-[5,7-difluoro-2-(4-fluorophenyl)-1H-indol-3-yl]propanoic acid) (S8)

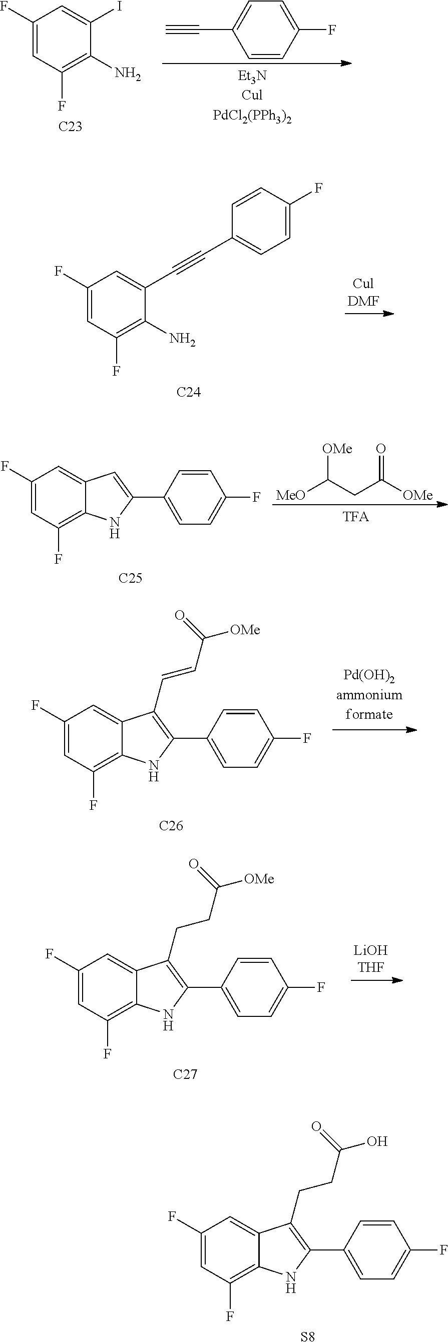

### Step 1. Synthesis of 2,4-difluoro-6-[2-(4-fluorophenyl)ethynyl]aniline (C24)

To a flask containing 2,4-difluoro-6-iodo-aniline C23 (134 g, 525.5 mmol) was added NEt₃ (1.3 L), followed by DMF (250 mL), 1-ethynyl-4-fluoro-benzene (83.5 g, 695.1 mmol), CuI (20.5 g, 107.6 mmol), and PdCl₂(PPh₃)₂ (25 g, 35.6 mmol). The mixture was allowed to stir at room temperature for 2 hours. Solvent was removed under reduced pressure and water (500 mL) was added. The mixture was extracted with Ethyl acetate, filtered and concentrated in vacuo. The product mixture was filtered through a silica gel plug (Eluent: CH₂Cl₂), followed by a second silica plug filtration (Eluent: 30-40% EtOAc in heptane). Silica gel chromatography (Gradient: 0-20% EtOAc in heptane) afforded the product as a pale yellow solid. (87 g, 60%). ¹H NMR (300 MHz, Chloroform-d) δ 7.58-7.45 (m, 2H), 7.14-7.02 (m, 2H), 6.92 (ddd, J=8.8, 2.8, 1.7 Hz, 1H), 6.87-6.71 (m, 1H), 4.15 (s, 2H). LCMS m/z 248.0 [M+H]⁺.

### Step 2. Synthesis of 5,7-difluoro-2-(4-fluorophenyl)-1H-indole (C25)

To a solution of 2,4-difluoro-6-[2-(4-fluorophenyl)ethynyl]aniline C24 (46 g, 167.5 mmol) in DMF (600 mL) was added CuI (1.9 g, 10.0 mmol) and the reaction was heated at reflux. Water (800 mL) was added and the mixture extracted with MTBE. The mixture was then washed with sat. NaCl solution, dried over Na₂SO₄ and then concentrated in vacuo to afford the product, which was used in subsequent steps without further purification (41 g, 87%). ¹H NMR (300 MHz, Chloroform-d) δ 8.43 (s, 1H), 7.72-7.58 (m, 2H), 7.27-7.15 (m, 2H), 7.09 (dd, J=9.0, 2.1 Hz, 1H), 6.85-6.63 (m, 2H). LCMS m/z 248.0 [M+H]⁺.

### Step 3. Synthesis of methyl (E)-3-[5,7-difluoro-2-(4-fluorophenyl)-1H-indol-3-yl]prop-2-enoate (C26)

A 12 L flask with overhead stirrer was charged with 5,7-difluoro-2-(4-fluorophenyl)-1H-indole C25 (300 g, 1.2 mol), CH₂Cl₂ (3 L), methyl 3,3-dimethoxypropanoate (195 mL, 1.4 mol) and TFA (300 mL, 3.9 mol). The reaction was heated to reflux for 4 hours. Additional CH₂Cl₂ was added to facilitate stirring. Upon cooling to room temperature, the solid product was filtered, washed with minimal CH₂Cl₂ and dried to afford the product (388 g, 96%). ¹H NMR (400 MHz, DMSO-d₆) δ 12.66 (s, 1H), 7.77-7.57 (m, 4H), 7.56-7.37 (m, 2H), 7.19 (ddd, J=11.0, 9.7, 2.1 Hz, 1H), 6.47 (d, J=16.1 Hz, 1H), 3.69 (s, 3H). LCMS m/z 332.4 [M+H]⁺.

### Step 4. Synthesis of methyl 3-[5,7-difluoro-2-(4-fluorophenyl)-1H-indol-3-yl]propanoate (C27)

To a suspension of methyl (E)-3-[5,7-difluoro-2-(4-fluorophenyl)-1H-indol-3-yl]prop-2-enoate C26 (80 g, 236.5 mmol) in EtOH (1.5 L) under a nitrogen atmosphere was added Pd(OH)₂ (6 g of 20% w/w 8.5 mmol) and ammonium formate (160 g, 2.5 mol). The mixture was heated at reflux for ˜3 hours, then filtered to remove catalyst. The filtrate was concentrated in vacuo to afford the product as an off-white solid which was used without further purification (82 g, 100%). ¹H NMR (300 MHz, Chloroform-d) δ 8.18 (s, 1H), 7.65-7.47 (m, 2H), 7.27-7.14 (m, 2H), 7.14-7.00 (m, 1H), 6.76 (ddd, J=10.8, 9.4, 2.2 Hz, 1H), 3.65 (s, 3H), 3.27-3.04 (m, 2H), 2.75-2.49 (m, 2H). LCMS m/z 334.3 [M+H]⁺.

### Step 5. Synthesis of 3-[5,7-difluoro-2-(4-fluorophenyl)-1H-indol-3-yl]propanoic acid (S8)

LiOH (67 g, 2.8 mol) was added to a solution of methyl 3-[5,7-difluoro-2-(4-fluorophenyl)-1H-indol-3-yl]propanoate C27 (217 g, 651.1 mmol) in THF (1 L) and water (100 mL). The mixture was heated at reflux for 2 hours, and then allowed to cool overnight. THF was removed by concentration under reduced pressure, and water was added (approx. 1 L). The mixture was cooled on an ice bath and HCl (250 mL of 11.7 M, 2.9 mol) was added to adjust pH to ˜4. EtOAc (300 mL) was added, and the aqueous layer extracted with further EtOAc (100 mL). Combined organic extracts were dried over sodium sulfate (Na₂SO₄), filtered through a plug of silica gel rinsing with EtOAc. The filtrate was concentrated in vacuo to afford an orange oil (50-75 mL). Heptanes (˜50 mL) were added and the mixture chilled on dry ice. Upon agitation, a crystalline solid formed. The mixture was allowed to stir on an ice-bath until to allow completion of the crystallization process. The solid was filtered, washed with heptane and air dried to afford the product (S8) (208 g, 96%). ¹H NMR (300 MHz, Chloroform-d) δ 8.15 (s, 1H), 7.60-7.46 (m, 2H), 7.27-7.15 (m, 2H), 7.09 (dd, J=9.1, 2.2 Hz, 1H), 6.77 (ddd, J=10.8, 9.4, 2.2 Hz, 1H), 3.26-3.05 (m, 2H), 2.78-2.57 (m, 2H). LCMS m/z 320.0 [M+H]⁺.

### Preparation S8 (Alternative Preparation)

### Step 3. Synthesis of methyl (E)-3-[5,7-difluoro-2-(4-fluorophenyl)-1H-indol-3-yl]prop-2-enoate (C26)

A reactor was charged with 5,7-difluoro-2-(4-fluorophenyl)-1H-indole C25 (4.0 kg, 16.5 mol), CH₂Cl₂ (37 L) and methyl 3,3-dimethoxypropanoate (2.6 L, 18.1 mol) followed by TFA (3.9 L, 51.0 mol) at ambient temperature. The resulting mixture was heated to reflux for 6 hours. The batch was then cooled to 20° C., charged with n-heptane (2 vol) and filtered. The filter cake was dried under vacuum at 45° C. to afford the product in ˜90% yield. ¹H NMR (300 MHz, DMSO-d₆) δ 12.63 (s, 1H), 7.76-7.54 (m, 4H), 7.55-7.39 (m, 2H), 7.18 (ddd, J=11.1, 9.7, 2.2 Hz, 1H), 6.46 (d, J=16.1 Hz, 1H), 3.69 (s, 3H). LCMS m/z 332.1 [M+H]⁺.

### Step 4. Synthesis of methyl 3-[5,7-difluoro-2-(4-fluorophenyl)-1H-indol-3-yl]propanoate (C27)

Methyl (E)-3-[5,7-difluoro-2-(4-fluorophenyl)-1H-indol-3-yl]prop-2-enoate C26 (1.5 kg, 9.06 mol) was slurried with THF (7 L) in a vessel. Pd(OH)₂ (10 g of 20% w/w, ˜50% water, 0.014 mol) was charged. The mixture was purged with N₂ three times, then once with H₂ and the vessel pressurized to 50 psi with H₂. The mixture was agitated at 20° C. until H₂ uptake ceased. After 1.5 hours, the mixture was purged with N₂ (3 times) and filtered through Solka-Floc using a THF (2 vol) rinse. The resulting filtrate was concentrated in vacuo at 45° C. (to 1.5 vol), charged with cyclohexane (1 vol), and concentrated again (to 1.5 vol) at 45° C. The slurry was cooled to 15-20° C. and filtered. The filter cake was then washed with cold cyclohexane (1 vol) and dried under vacuum at 45° C. to afford the product in 95% yield.

### Step 5. Synthesis of 3-[5,7-difluoro-2-(4-fluorophenyl)-1H-indol-3-yl]propanoic acid (S8)

A mixture of methyl 3-[5,7-difluoro-2-(4-fluorophenyl)-1H-indol-3-yl]propanoate C27 (9 kg, 27 mmol) in 2-MeTHF (54 L, 6 vol) and MeOH (8.1 L, 0.9 vol) was charged with 20% KOH (2 equiv, 54 mol). The mixture was stirred at 35° C. for 6 hours. The mixture was then distilled under vacuum to 27 L (3 vol) and cooled to 10-15° C. Water (7.5 L) and 2-MeTHF (16 L) were charged and the resulting biphasic mixture was pH adjusted with 6 M HCl to a pH ˜2. The temperature was adjusted to 20° C. and the phases separated. The organic phase was washed with water (15 L), filtered through Celite® with 2-MeTHF rinse (18 L, 2 vol), and concentrated under vacuum to 18 L (2 vol). 18 L (2 vol) of n-heptane was charged and the batch again concentrated under vacuum to 18 L (3 vol). This cycle was repeated once more, and the batch was seeded. 16 L (1.8 vol) n-heptane was charged and the temperature adjusted to 20° C. The slurry was stirred for 2 hours, filtered and the cake washed with 2×18 L (2×2 vol) n-heptane. The filter cake was dried under vacuum at 45° C. to afford the desired product in 90% yield. ¹H NMR (300 MHz, Chloroform-d) δ 8.28 (s, 1H), 7.53 (ddd, J=8.7, 5.4, 2.8 Hz, 2H), 7.27-7.13 (m, 2H), 7.08 (dd, J=9.1, 2.1 Hz, 1H), 6.76 (ddd, J=11.3, 9.4, 2.2 Hz, 1H), 3.91-3.69 (m, 4H), 3.28-3.07 (m, 2H), 2.79-2.53 (m, 2H), 2.00-1.74 (m, 3H). LCMS m/z 320.4 [M+H]⁺.

### Preparation S9

### 3-[5-chloro-7-fluoro-2-(4-fluorophenyl)-1H-indol-3-yl]propanoic acid (S9)

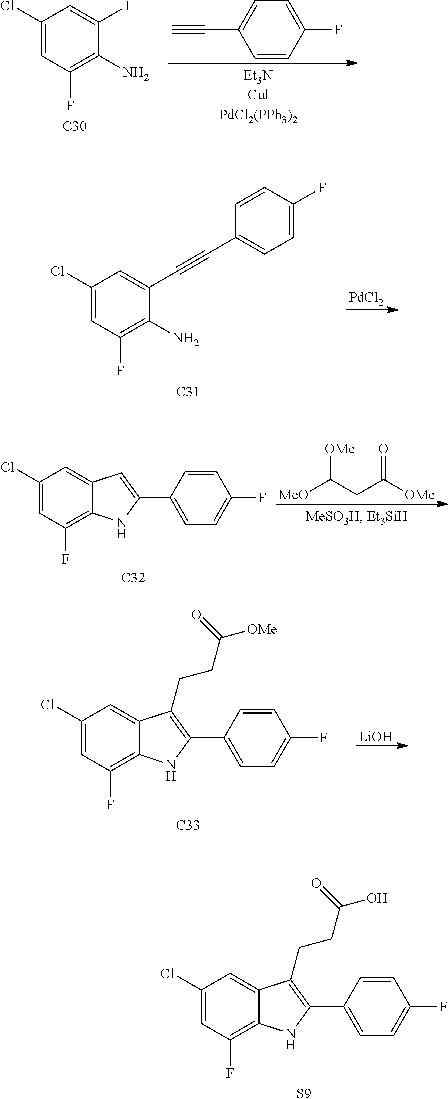

### Step 1. Synthesis of 4-chloro-2-fluoro-6-[2-(4-fluorophenyl)ethynyl]aniline (C31)

4-Chloro-2-fluoro-6-iodo-aniline (5 g, 17.498 mmol) was added to a solution of 1-ethynyl-4-fluoro-benzene C30 (2.7601 g, 2.6287 mL, 22.747 mmol) and triethylamine (4.0929 g, 5.6376 mL, 40.245 mmol) in DMF (100 mL) at room temperature and stirred for 1 hour. Water (150 mL) was added, and the mixture was stirred for 0.5 hours. The precipitate was filtrated and washed with water (100 mL). The crude material was purified by silica gel chromatography (Gradient: 0-20% EtOAc in hexane) to give 4-chloro-2-fluoro-6-[2-(4-fluorophenyl)ethynyl]aniline C31 (5.1 g, 99%) as a brown solid. ¹H NMR (300 MHz, DMSO-d₆) δ 7.77-7.69 (m, 2H), 7.31-7.22 (m, 3H), 7.16 (s, 1H), 5.75 (s, 2H). LCMS m/z 264.1 [M+H]⁺.

### Step 2. Synthesis of 5-chloro-7-fluoro-2-(4-fluorophenyl)-1H-indole (C32)

4-chloro-2-fluoro-6-[2-(4-fluorophenyl)ethynyl]aniline C31 (4.85 g, 16.555 mmol) and palladium(II) chloride (593.06 mg, 3.3110 mmol) were suspended in acetonitrile (485.00 mL) and stirred at 80° C. under argon atmosphere for 5 hours. Then acetonitrile was evaporated under reduced pressure (2 mbar, 40° C.). The residue was dissolved in DCM (200 mL) and washed with water (100 mL) and brine (100 mL). Organic layer was dried over MgSO4 and concentrated to dryness. The crude material (6.1 g) was purified by column chromatography (Gradient: 0-4% EtOAc in hexane) to give 5-chloro-7-fluoro-2-(4-fluorophenyl)-1H-indole C32 (3.7 g, 81%), as a yellow solid. ¹H NMR (300 MHz, DMSO-d₆) δ 12.08 (s, 1H), 7.98 (dd, J=8.7, 5.4 Hz, 2H), 7.44 (s, 1H), 7.32 (t, J=8.8 Hz, 2H), 7.07 (d, J=10.8 Hz, 1H), 6.96 (d, J=3.3 Hz, 1H). LCMS m/z 264.2 [M+H]⁺.

### Step 3. Synthesis of methyl 3-[5-chloro-7-fluoro-2-(4-fluorophenyl)-1H-indol-3-yl]propanoate (C33)

To 5-chloro-7-fluoro-2-(4-fluorophenyl)-1H-indole C32 (654 mg, 2.480 mmol) in toluene (10 mL) was added methanesulfonic acid (251 μL, 3.868 mmol) and triethylsilane (1.3 mL, 8.139 mmol) followed by methyl 3,3-dimethoxypropanoate (440 μL, 3.103 mmol). The reaction was heated at 70° C. for 2 hours. Water (50 mL) was added, and the aqueous layer was extracted with EtOAc (3×30 mL). The combined organic layer was dried over MgSO4 and concentrated to dryness. The crude material was purified by silica gel chromatography (Gradient: 0-100% EtOAc in hexane) to provide methyl 3-[5-chloro-7-fluoro-2-(4-fluorophenyl)-1H-indol-3-yl]propanoate C33 (528 mg, 59%). ¹H NMR (300 MHz, Methanol-d₄) δ 7.76-7.50 (m, 2H), 7.37 (d, J=1.7 Hz, 1H), 7.30-7.10 (m, 2H), 6.90 (dd, J=10.8, 1.7 Hz, 1H), 3.57 (s, 3H), 3.25-3.00 (m, 2H), 2.60 (dd, J=8.4, 7.0 Hz, 2H). LCMS m/z 350.16 [M+H]⁺.

### Step 4. Synthesis of 3-[5-chloro-7-fluoro-2-(4-fluorophenyl)-1H-indol-3-yl]propanoic acid (S9)

A mixture of methyl 3-[5-chloro-7-fluoro-2-(4-fluorophenyl)-1H-indol-3-yl]propanoate C33 (526 mg, 1.470 mmol) and LiOH (353 mg, 14.74 mmol) in methanol (4 mL), THF (4 mL), and water (4 mL) was stirred at 60° C. for 3 hours. The reaction mixture was concentrated, and added water (50 mL), then acidified with concentrated HCl to pH=1. The aqueous layer was extracted with DCM (3×50 mL). The combined organic layer was washed with brine, dried over Na₂SO₄, and evaporated to afford product S9 (545 mg, quantitative). LCMS m/z 335.99 [M+H]⁺.

### Preparation S10

### 3-(7-fluoro-2-(4-fluorophenyl)-5-methyl-1H-indol-3-yl)propanoic acid (S10)

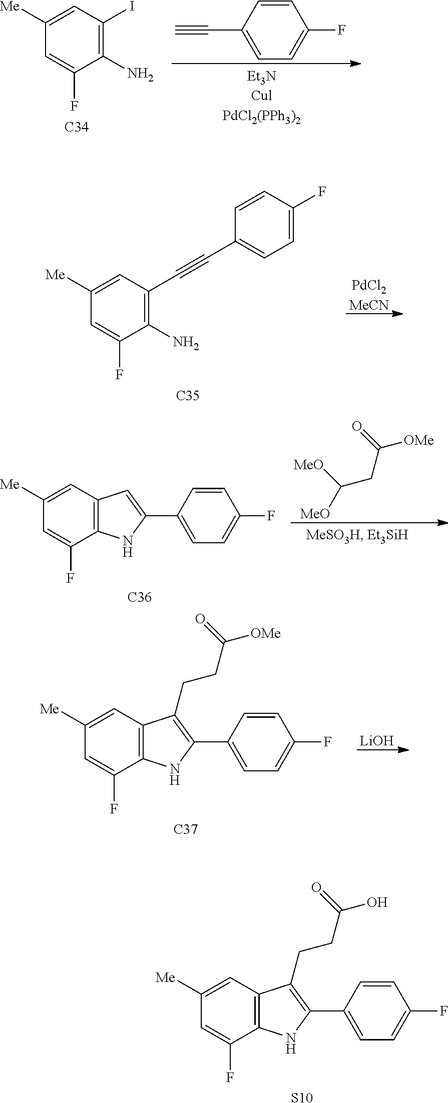

### Step 1. Synthesis of 2-fluoro-6-[2-(4-fluorophenyl)ethynyl]-4-methyl-aniline (C35)

To a mixture of 1-ethynyl-4-fluoro-benzene (1.74 g, 1.66 mL, 14.3 mmol), Et₃N (2.58 g, 3.56 mL, 25.4 mmol), CuI (343 mg, 1.77 mmol) and PdCl₂(PPh₃)₂ (395 mg, 0.55 mmol) in DMF (55 mL) was added 2-fluoro-6-iodo-4-methyl-aniline C34 (2.77 g, 11.0 mmol). The resulting suspension was stirred at ambient temperature for 2 hours. The reaction was quenched with H₂O (100 mL) and extracted with DCM (3×100 mL). The combined organic extracts were dried over MgSO4, filtered and concentrated to afford the title compound as a brown solid (3.2 g, 95%). The crude was used in the subsequent step without further purification. ¹H NMR (300 MHz, DMSO-d₆) δ 7.73-7.65 (m, 2H), 7.26 (t, J=8.9 Hz, 2H), 6.92 (t, J=5.8 Hz, 2H), 5.28 (s, 2H), 2.15 (s, 3H). LCMS m/z 244.1 [M+H]⁺.

### Step 2. Synthesis of 7-fluoro-2-(4-fluorophenyl)-5-methyl-1H-indole (C36)

2-Fluoro-6-[2-(4-fluorophenyl)ethynyl]-4-methyl-aniline C35 (3.2 g, 10.5 mmol) and PdCl₂ (189 mg, 1.05 mmol) were suspended in acetonitrile (320 mL) under an argon atmosphere. The reaction was stirred at 80° C. for 5 hours. The volatile was then removed in vacuo. The crude was dissolved in DCM (100 mL) and washed with H₂O (50 mL) and brine (50 mL). The organic layer was separated and dried over MgSO4, filtered and concentrated. The crude was purified using silica gel chromatography (0-2% EtOAc in hexanes) to afford C36 (1.82 g, 69%). ¹H NMR (300 MHz, DMSO-d₆) δ 11.69 (s, 1H), 7.96 (dd, J=8.7, 5.4 Hz, 2H), 7.29 (t, J=8.8 Hz, 2H), 7.13 (s, 1H), 6.86 (t, J=2.8 Hz, 1H), 6.76 (d, J=12.4 Hz, 1H), 2.36 (s, 3H). LCMS m/z 244.2 [M+H]⁺.

### Step 3. Synthesis of methyl 3-[7-fluoro-2-(4-fluorophenyl)-5-methyl-1H-indol-3-yl]propanoate (C37)

To a solution of 7-fluoro-2-(4-fluorophenyl)-5-methyl-1H-indole C36 (518 mg, 2.13 mmol) in toluene (10 mL) was added sequentially methanesulfonic acid (220 μL, 3.39 mmol), triethylsilane (1.2 mL, 7.51 mmol) and methyl 3,3-dimethoxypropanoate (380 μL, 2.68 mmol). The resulting mixture was stirred at 80° C. overnight. The reaction was then cooled down to ambient temperature and quenched with H₂O (50 mL), extracted with EtOAc (3×30 mL). The combined organic extracts were concentrated. The crude was purified using silica gel chromatography (0-100% EtOAc in hexanes) to afford the title compound as an off-white solid C37 (481 mg, 69%). ¹H NMR (400 MHz, Chloroform-d) δ 8.05 (s, 1H), 7.59-7.47 (m, 2H), 7.26-7.11 (m, 3H), 6.79 (dd, J=12.0, 1.2 Hz, 1H), 3.67 (s, 3H), 3.28-3.11 (m, 2H), 2.76-2.61 (m, 2H), 2.50 (s, 3H). LCMS m/z 330.1 [M+H]⁺.

### Step 4. Synthesis of 3-(7-fluoro-2-(4-fluorophenyl)-5-methyl-1H-indol-3-yl)propanoic acid (S10)

To a solution of methyl 3-[7-fluoro-2-(4-fluorophenyl)-5-methyl-1H-indol-3-yl]propanoate C₃₇ (480 mg, 1.46 mmol) in MeOH (4 mL), THF (4 mL) and H₂O (4 mL) was added LiOH (480 mg, 1.46 mmol). The resulting mixture was stirred at 60° C. for 3 hours. The volatile was removed and H₂O (50 mL) was added. The pH of the reaction mixture was adjusted to 1 using concentrated HCl. The reaction was extracted with DCM (3×50 mL) and the combined organic extracts were washed with brine, dried over Na₂SO₄, filtered and concentrated to afford S10 (489 mg, 100%). LCMS m/z 316.1 [M+H]⁺.

### Preparation S11

### 3-(5-bromo-7-fluoro-2-(4-fluorophenyl)-1H-indol-3-yl) propanoic acid (S11)

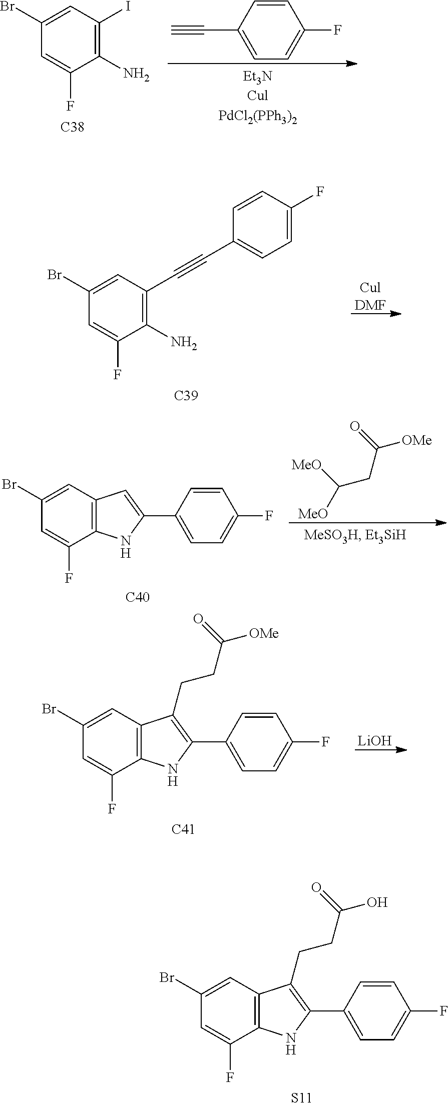

### Step 1. Synthesis of 4-bromo-2-fluoro-6-[2-(4-fluorophenyl) ethynyl] aniline (C39)

To a solution of 4-bromo-2-fluoro-6-iodo-aniline C38 (40 g, 126.6 mmol) in DMF (80 mL) was added TEA (400 mL), 1-ethynyl-4-fluoro-benzene (20 g, 166.5 mmol), iodocopper (4 g, 21.00 mmol), and PdCl₂(PPh₃)₂ (4.6 g, 6.6 mmol). Reaction was stirred at room temperature for 5 hours. Solvent was removed under reduced pressure and water (500 mL) was added. The mixture was extracted with MTBE, filtered and concentrated in vacuo. The product mixture was filtered through a silica gel plug (Eluent: CH₂Cl₂), followed by a second silica plug filtration (Eluent 20% EtOAc in heptane). Silica gel chromatography (Gradient: 0-15% EtOAc in heptane) afforded the product 4-bromo-2-fluoro-6-[2-(4-fluorophenyl) ethynyl] aniline (C39) (32 g, 66%). ¹H NMR (300 MHz, Chloroform-d) δ 7.63-7.42 (m, 2H), 7.34-7.25 (m, 1H), 7.22-6.97 (m, 3H), 4.31 (s, 2H). LCMS m/z 308.21 [M+H]⁺.

### Step 2. Synthesis of 5-bromo-7-fluoro-2-(4-fluorophenyl)-1H-indole) (C40)

A solution of 4-bromo-2-fluoro-6-[2-(4-fluorophenyl) ethynyl] aniline C39 (32 g, 103.9 mmol) in DMF (400 mL) was heated to 150° C. for 4 hours. The reaction was then stirred at room temperature overnight. Iodocopper (2 g, 10.50 mmol) was added and the reaction was heated to 150° C. for 3 hours. Reaction was cooled and water (800 mL) was added. The mixture was extracted with MTBE, washed with sat. NaCl and water, dried over sodium sulfate, filtered and concentrated in vacuo. The product mixture was filtered through a silica gel plug (Eluent 20% EtOAc in heptane). Silica gel chromatography (Gradient: 0-20% in heptane) afforded the product 5-bromo-7-fluoro-2-(4-fluorophenyl)-1H-indole (C40) (14.4 g, 45%). ¹H NMR (300 MHz, Chloroform-d) δ 8.47 (s, 1H), 7.73-7.59 (m, 2H), 7.55 (dd, J=1.5, 0.7 Hz, 1H), 7.26-7.14 (m, 2H), 7.08 (dd, J=10.2, 1.6 Hz, 1H), 6.73 (dd, J=3.4, 2.3 Hz, 1H). LCMS m/z 307.01 [M+H]⁺.

### Step 3. Synthesis of methyl 3-[5-bromo-7-fluoro-2-(4-fluorophenyl)-1H-indol-3-yl]propanoate (C41)

To a solution of 5-bromo-7-fluoro-2-(4-fluorophenyl)-1H-indole C40 (966 mg, 3.131 mmol) in toluene (10 mL) was added methane sulfonic acid (320 μL, 4.931 mmol) and triethylsilane (1.55 mL, 9.704 mmol) followed by methyl 3,3-dimethoxypropanoate (536 μL, 3.781 mmol). Reaction was heated at 70° C. for 2 hours. Water was added (50 mL), and the organic layer was extracted with EtOAc (3×30 mL), dried over sodium sulfate, filtered and concentrated in vacuo. Silica gel chromatography (Gradient: EtOAc in heptane) afforded the product methyl 3-[5-bromo-7-fluoro-2-(4-fluorophenyl)-1H-indol-3-yl]propanoate (C41) (890 mg, 72%). ¹H NMR (300 MHz, Chloroform-d) δ 8.19 (s, 1H), 7.61-7.46 (m, 3H), 7.22 (dd, J=8.9, 8.4 Hz, 2H), 7.10 (dd, J=10.2, 1.5 Hz, 1H), 3.66 (s, 3H), 3.25-3.05 (m, 2H), 2.75-2.51 (m, 2H). LCMS m/z 394.04 [M+H]⁺.

### Step 4. Synthesis of 3-[5-bromo-7-fluoro-2-(4-fluorophenyl)-1H-indol-3-yl]propanoic acid (S11)

A solution of methyl 3-[5-bromo-7-fluoro-2-(4-fluorophenyl)-1˜{H}-indol-3-yl]propanoate C41 (520 mg, 1.319 mmol) and LiOH (500 mg, 20.88 mmol) in MeOH (5 mL), THF (8 mL) and water (10 mL) was stirred and heated at 50° C. overnight. Organic solvent was evaporated and water (100 mL) was added. The solution was acidified to pH 1 by adding concentrated HCl. The aqueous layer was extracted with DCM. The combined organic layers were washed with brine, dried over anhydrous sodium sulfate, filtered and concentrated in vacuo to give 3-[5-bromo-7-fluoro-2-(4-fluorophenyl)-1H-indol-3-yl]propanoic acid (S11) (599 mg, quantitative). ¹H NMR (300 MHz, Chloroform-d) δ 8.10 (s, 1H), 7.52-7.35 (m, 3H), 7.17-7.07 (m, 2H), 7.01 (dd, J=10.2, 1.5 Hz, 1H), 3.14-2.95 (m, 2H), 2.68-2.47 (m, 2H). LCMS m/z 380.09 [M+H]⁺.

### Preparation S12

### 3-[4,7-difluoro-2-(4-fluorophenyl)-1H-indol-3-yl]propanoic acid (S12)

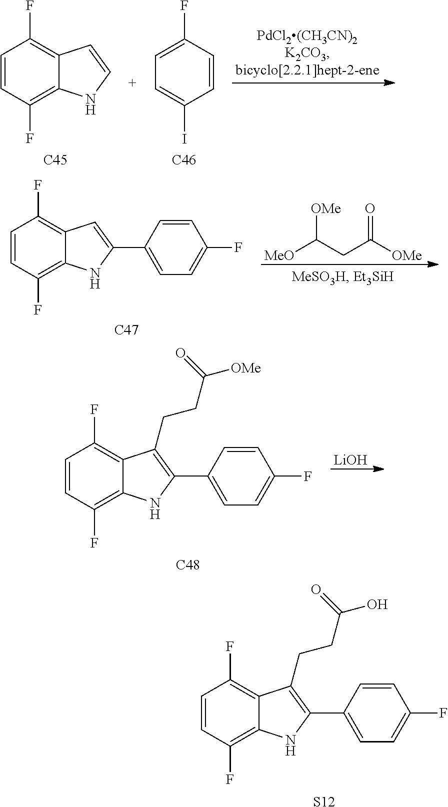

### Step 1. Synthesis of 4,7-difluoro-2-(4-fluorophenyl)-1H-indole (C47)

To a solution of 4,7-difluoro-1H-indole (500 mg, 3.265 mmol) C45 and 1-fluoro-4-iodo-benzene (C46) (490 μL, 4.249 mmol) in DMA (4 mL) and water (1 mL) was added K₂CO₃ (1.2 g, 8.683 mmol), bicyclo[2.2.1]hept-2-ene (923 mg, 9.803 mmol), and Bis(acetonitrile)dichloropalladium(II) (85 mg, 0.3276 mmol). The reaction was warmed to 90° C. The reaction was heated for 14 hours before being allowed to cool to room temperature. The reaction was diluted with water and extracted with EtOAc. The organic extract was dried with MgSO4, filtered and concentrated in vacuo. Purification by silica gel chromatography (Eluent: 10% EtOAc in heptane) afforded the product 4,7-difluoro-2-(4-fluorophenyl)-1H-indole (C47) (689 mg, 84%). ¹H NMR (300 MHz, Chloroform-d) δ 8.48 (s, 1H), 7.75-7.56 (m, 2H), 7.25-7.06 (m, 2H), 6.93-6.77 (m, 2H), 6.74-6.50 (m, 1H). LCMS m/z 248.13 [M+H]⁺.

### Step 2. Synthesis of methyl 3-[4,7-difluoro-2-(4-fluorophenyl)-1H-indol-3-yl]propanoate (C48)

To 4,7-difluoro-2-(4-fluorophenyl)-1H-indole C47 (383 mg, 1.532 mmol) in toluene (10 mL) was added methanesulfonic acid (155 μL, 2.389 mmol) and triethylsilane (750 μL, 4.696 mmol) followed by methyl 3,3-dimethoxypropanoate (262 μL, 1.848 mmol). The reaction was heated at 70° C. for 12 hours. Water (50 mL) was added, and the aqueous layer was extracted with EtOAc (3×30 mL). The combined organic layer was dried over MgSO4 and concentrated to dryness. The crude material was purified by silica gel chromatography (Gradient: 0-100% EtOAc in hexane) to provide methyl 3-[4,7-difluoro-2-(4-fluorophenyl)-1H-indol-3-yl]propanoate (C48) (48 mg, 9%). ¹H NMR (300 MHz, Chloroform-d) δ 8.18 (s, 1H), 7.63-7.51 (m, 2H), 7.27-7.16 (m, 2H), 6.82 (ddd, J=10.0, 8.6, 3.5 Hz, 1H), 6.69 (ddd, J=10.3, 8.6, 3.3 Hz, 1H), 3.64 (s, 3H), 3.35-3.16 (m, 2H), 2.82-2.62 (m, 2H). LCMS m/z 334.01 [M+H]⁺.

### Step 3. Synthesis of 3-[4,7-difluoro-2-(4-fluorophenyl)-1H-indol-3-yl]propanoic acid (S12)

A mixture of methyl 3-[4,7-difluoro-2-(4-fluorophenyl)-1H-indol-3-yl]propanoate C48 (47 mg, 0.1402 mmol) and LiOH (68 mg, 2.839 mmol) in MeOH (5 mL), THF (4 mL) and water (4 mL) was stirred at 60° C. for 20 hours. The reaction mixture was concentrated, and water (50 mL) was added. The solution was acidified with concentrated HCl to pH=1. The aqueous layer was extracted with DCM (3×30 mL). The combined organic layer was washed with brine, dried over Na₂SO₄, and evaporated to afford product 3-[4,7-difluoro-2-(4-fluorophenyl)-1H-indol-3-yl]propanoic acid (S12) (52 mg, 100%). ¹H NMR (300 MHz, Chloroform-d) δ 8.12 (s, 1H), 7.51-7.35 (m, 2H), 7.11 (t, J=8.5 Hz, 2H), 6.73 (td, J=9.3, 3.4 Hz, 1H), 6.60 (ddd, J=11.8, 8.6, 3.3 Hz, 1H), 3.12 (t, J=8.0 Hz, 2H), 2.67 (t, J=8.0 Hz, 2H). LCMS m/z 320.1 [M+H]⁺.

### Preparation S13

### 3-[2-(4-cyanophenyl)-5,7-difluoro-1H-indol-3-yl]propanoic acid (S13)

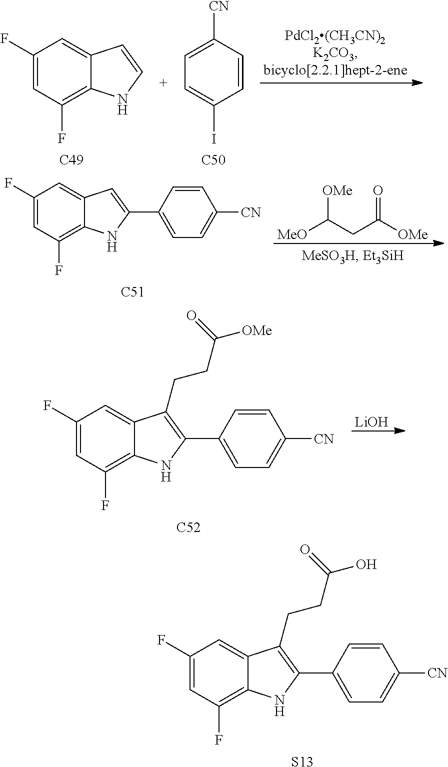

### Step 1. Synthesis of 4-(5,7-difluoro-1H-indol-2-yl)benzonitrile (C51)

A solution of 5,7-difluoro-1H-indole C49 (3.0 g, 19.59 mmol), 4-iodobenzonitrile C50 (4.9 g, 21.40 mmol), DMA (35 mL), water (4 mL), K₂CO₃ (5.64 g, 40.81 mmol), bicyclo[2.2.1]hept-2-ene (3.1 g, 32.92 mmol), and acetonitrile; dichloropalladium (421 mg, 1.623 mmol) was warmed to 90° C. and stirred overnight. The reaction was cooled to room temperature. The reaction was diluted with water (75 mL) and was extracted with ethyl acetate (2×50 mL). The combined organics were washed with brine, dried with MgSO4, filtered, and concentrated in vacuo. Purification by silica gel chromatography (Eluent: 10% EtOAc in heptane) afforded the product 4-(5,7-difluoro-1H-indol-2-yl)benzonitrile (C51) (2.79 g, 52%). LCMS m/z 254.97 [M+H]⁺.

### Step 2. Synthesis of 3-[2-(4-cyanophenyl)-5,7-difluoro-1H-indol-3-yl]propanoate (C52)

4-(5,7-difluoro-1H-indol-2-yl)benzonitrile C51 (101 mg, 0.371 mmol), methyl 3,3-dimethoxypropanoate (79 μL, 0.5572 mmol), methanesulfonic acid (49 μL, 0.7551 mmol) and triethylsilane (180 μL, 1.127 mmol) were added to diethyl carbonate (2 mL) in a microwave tube. The reaction mixture was subjected to the microwave at 180° C. for 1 hour. Additional methyl 3,3-dimethoxypropanoate (79 μL, 0.5572 mmol), methanesulfonic acid (49 μL, 0.7551 mmol), triethylsilane (180 μL, 1.127 mmol) and TFA (58 μL, 0.7528 mmol) was added and the reaction was reheated to 180° C. for 2 hours in the microwave. The reaction was cooled to room temperature and water (100 mL) was added. Aqueous layer was extracted with EtOAc (3×50 mL). The combined organic layer was dried over MgSO4 and concentrated to dryness. Purification by silica gel chromatography (Gradient: 0-100% EtOAc in hexane) afforded methyl 3-[2-(4-cyanophenyl)-5,7-difluoro-1H-indol-3-yl]propanoate (C52) (16 mg, 12%). LCMS m/z 341.07 [M+H]⁺.

### Step 3. Synthesis of 3-[2-(4-cyanophenyl)-5,7-difluoro-1H-indol-3-yl]propanoic acid (S13)

To a solution of methyl 3-[2-(4-cyanophenyl)-5,7-difluoro-1H-indol-3-yl]propanoate C52 (16 mg, 0.04436 mmol) in MeOH (1 mL) and THF (2 mL) was added aqueous LiOH solution (1 mL). The reaction mixture was stirred at 50° C. for 3 h. The reaction mixture was cooled to room temperature, concentrated, and water (40 mL) was added. The reaction was acidified with concentrated HCl to pH=1. The aqueous layer was extracted with DCM (3×25 mL). The combined organic layer was washed with brine, dried over Na₂SO₄, and evaporated to afford product 3-[2-(4-cyanophenyl)-5,7-difluoro-1H-indol-3-yl]propanoic acid (S13) (14 mg, 97%). LCMS m/z 327.03 [M+H]⁺.

### Preparation S14

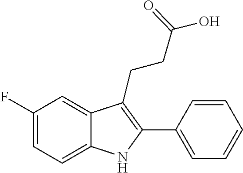 [SMILES: O=C(O)CCc1c(-c2ccccc2)[nH]c2ccc(F)cc12]

3-(5-fluoro-2-phenyl-1H-indol-3-yl)propanoic acid (S14) was obtained from commercial sources.

### Preparation S15

### 3-(5-fluoro-2-phenyl-1H-indol-3-yl)propanoic acid (S15)

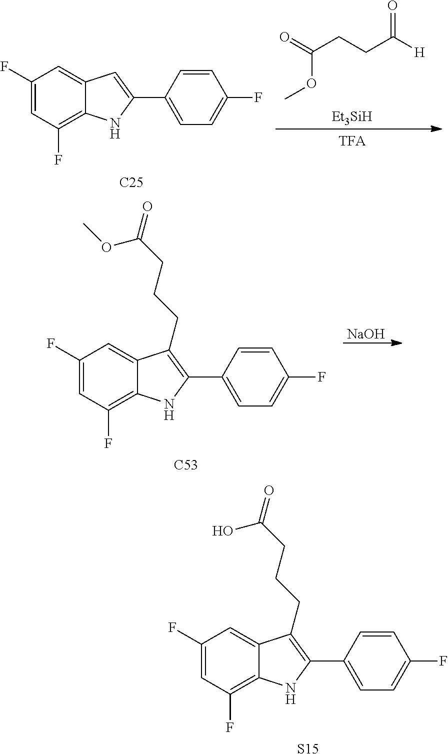

### Step 1. Synthesis of methyl 4-[5,7-difluoro-2-(4-fluorophenyl)-1H-indol-3-yl]butanoate (C53)

To a solution of methyl 4-oxobutanoate (1 g, 8.612 mmol) and 5,7-difluoro-2-(4-fluorophenyl)-1H-indole C25 (1.5 g, 6.068 mmol) in DCM (40 mL) was added Et₃SiH (4.8 mL, 30.05 mmol) and TFA (2.3 mL, 29.85 mmol). The mixture was stirred at room temperature overnight. Solvent was removed in vacuo. EtOAc and saturated NaHCO₃ aqueous solution were added. The aqueous layer was extracted with EtOAc (2×50 mL). The combined organic fractions were washed with NaHCO₃ and brine, dried over sodium sulfate, filtered, and the solution was concentrated to dryness. Purification by silica gel chromatography (Eluent: 50% EtOAc in heptane) afforded (C53) methyl 4-[5,7-difluoro-2-(4-fluorophenyl)-1H-indol-3-yl]butanoate (1.9 g, 76%). ¹H NMR (300 MHz, Chloroform-d) δ 8.34 (s, 1H), 7.51 (ddq, J=10.2, 5.0, 2.5, 2.0 Hz, 2H), 7.25-7.12 (m, 2H), 7.09 (dd, J=9.1, 2.2 Hz, 1H), 6.75 (ddd, J=10.8, 9.5, 2.2 Hz, 1H), 3.64 (s, 3H), 2.98-2.65 (m, 2H), 2.35 (t, J=7.2 Hz, 2H), 2.13-1.80 (m, 2H). LCMS m/z 348.2 [M+H]⁺.

### Step 2. Synthesis of 4-[5,7-difluoro-2-(4-fluorophenyl)-1H-indol-3-yl]butanoic acid (S15)

To a solution of methyl 4-[5,7-difluoro-2-(4-fluorophenyl)-1H-indol-3-yl]butanoate C53 (1.9 g, 5.5 mmol) in THF (30 mL) was added NaOH (30 mL of 2 M, 60 mmol). The reaction mixture was stirred at room temperature overnight. Organic layer was removed in vacuo, the pH was adjusted to 6. The aqueous layer was extracted with EtOAc. Combined organic fractions were washed with H₂O (20 mL), brine (20 mL), dried over sodium sulfate, filtered, and the solvent was removed in vacuo to obtain (S15) 4-[5,7-difluoro-2-(4-fluorophenyl)-1H-indol-3-yl]butanoic acid (1.7 g, 81%). ¹H NMR (300 MHz, Chloroform-d) δ 8.10 (s, 1H), 7.63-7.41 (m, 2H), 7.25-7.13 (m, 2H), 7.10 (dd, J=9.1, 2.2 Hz, 1H), 6.76 (ddd, J=10.8, 9.4, 2.1 Hz, 1H), 3.01-2.66 (m, 2H), 2.41 (t, J=7.1 Hz, 2H), 2.11-1.87 (m, 2H). LCMS m/z 334.25 [M+H]⁺.

### Preparation S16

### (S)-3-aminopyrrolidin-2-one (S16)

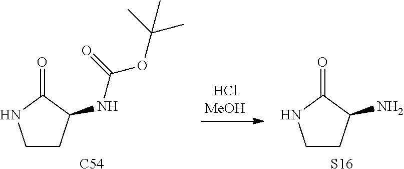

### Preparation of (S)-3-aminopyrrolidin-2-one (S16)

To a solution of tert-butyl N-[(3S)-2-oxopyrrolidin-3-yl]carbamate C54 (426 mg, 2.128 mmol) in methanol (4 mL) was added HCl (2 mL of 4 M, 8 mmol) in 1,4-dioxane. Reaction was stirred at 50° C. for 30 minutes. Solvent was evaporated and crude product was washed with ether to afford crude (S)-3-aminopyrrolidin-2-one (S16) (Hydrochloride salt) (302 mg, 99%). LCMS m/z 100.0 [M+H]⁺.

### Preparation S17

### (3S)-3-aminopyrrolidine-2,5-dione (S17)

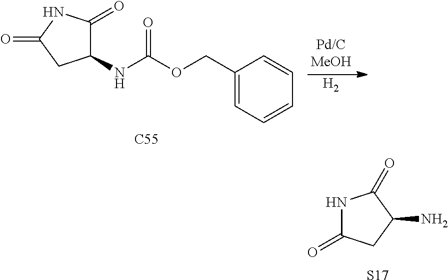

### Preparation of (3S)-3-aminopyrrolidine-2,5-dione (S17)

To a suspension benzyl N-[(3S)-2,5-dioxopyrrolidin-3-yl]carbamate C55 (649 mg, 2.614 mmol) in MeOH (10 mL) and EtOAc (5 mL) was added 5% palladium on carbon catalyst (20 mg). The mixture was subjected to hydrogenation conditions of 50 psi H₂ for 2 hours. Filtration through a pad of Celite®, washing with MeOH and CH₂Cl₂, then concentration of the filtrate in vacuo afforded (3S)-3-aminopyrrolidine-2,5-dione (S17) (398 mg, 98%). ¹H NMR (300 MHz, Chloroform-d) δ 4.00 (dd, J=8.7, 5.7 Hz, 1H), 3.12 (dd, J=18.2, 8.8 Hz, 1H), 2.69-2.37 (m, 1H), 1.73 (s, 2H). LCMS m/z 124.01 [M+H]⁺.

### Preparation S18

### (3S,5S)-3-amino-5-(hydroxymethyl)pyrrolidin-2-one (S18)

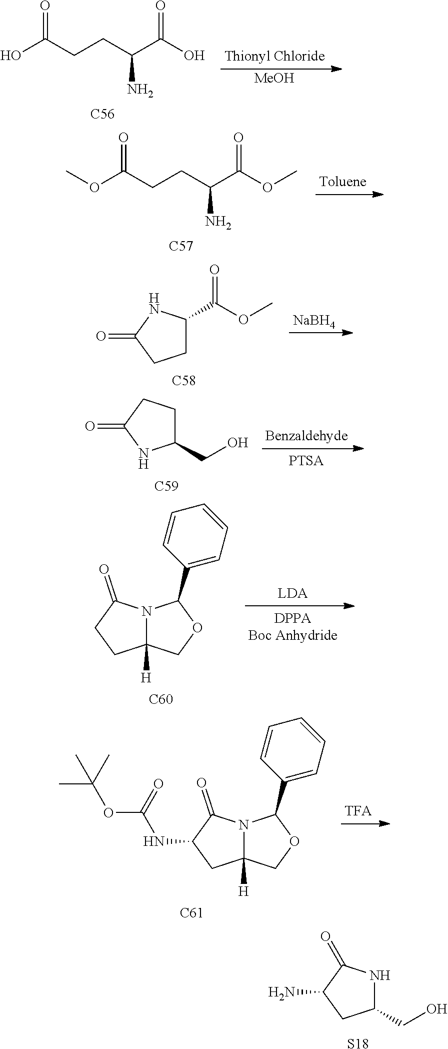

### Step 1. Synthesis of dimethyl (2S)-2-aminopentanedioate (C57)

To a stirred solution of (2S)-2-aminopentanedioic acid C56 (6 g, 40.781 mmol) in MeOH (30 mL) was added thionyl chloride (29.111 g, 17.849 mL, 244.69 mmol) dropwise under cooling conditions. The reaction mixture was stirred at room temperature for 20 hours. After completion reaction was concentrated in vacuo to remove excess thionyl chloride to afford dimethyl (2S)-2-aminopentanedioate (C57) (7 g, 98%) as colorless oily liquid. ¹H NMR (400 MHz, DMSO-d₆) δ 8.70 (s, 3H), 4.03 (t, J=6.6 Hz, 1H), 3.72 (s, 3H), 3.60 (s, 3H), 2.54 (dd, J=15.9, 8.3 Hz, 1H), 2.06 (q, J=7.4 Hz, 2H). One proton is obscured by the DMSO solvent peak.

### Step 2. Synthesis of methyl (2S)-5-oxopyrrolidine-2-carboxylate (C58)

A solution of dimethyl (2S)-2-aminopentanedioate C57 (16 g, 91.334 mmol) in toluene (150 mL) was stirred at reflux at 110° C. for 5 hours. Solvent was evaporated and purified by column-chromatography (eluted at 5% MeOH in DCM) to afford the desire product methyl (2S)-5-oxopyrrolidine-2-carboxylate (C58) (4.2 g, 32%). ¹H NMR (400 MHz, Chloroform-d) δ 6.14 (s, 1H), 4.32 (s, 1H), 3.78 (s, 3H), 2.52 (s, 1H), 2.40 (s, 2H), 2.29 (s, 1H).

### Step 3. Synthesis of (5S)-5-(hydroxymethyl)pyrrolidin-2-one (C59)

To a stirred solution of methyl (2S)-5-oxopyrrolidine-2-carboxylate C58 (4.2 g, 29.342 mmol) in IPA (40 mL) was added NaBH₄ (6.6604 g, 7.0480 mL, 176.05 mmol). The reaction was stirred at room temperature for 20 hours. Reaction was quenched by methanol and evaporated through reduced pressure. Purification by column chromatography (eluted at 5% MeOH in DCM) afforded (5S)-5-(hydroxymethyl)pyrrolidin-2-one (C59) (3.3 g, 98%). ¹H NMR (400 MHz, Chloroform-d) δ: 7.29 (s, 1H), 4.25 (s, 1H), 3.79-3.74 (m, 1H), 3.64 (d, J=11.8 Hz, 1H), 3.43 (t, J=10.2 Hz, 1H), 2.35-2.30 (m, 2H), 2.27-2.20 (m, 1H), 1.81-1.73 (m, 1H).

### Step 4. Synthesis of (3R,7aS)-3-phenyl-3,6,7,7a-tetrahydro-1H-pyrrolo[1,2-c]oxazol-5-one (C60)

To a stirred solution of (5S)-5-(hydroxymethyl)pyrrolidin-2-one C59 (3.3 g, 28.663 mmol) in toluene (45 mL) was added benzaldehyde (4.8669 g, 4.6797 mL, 45.861 mmol) and PTSA (272.62 mg, 1.4332 mmol) at room temperature. The reaction was stirred at room temperature for 17 hours. Reaction mixture was refluxed under Dean-Stark water separator for 6 hours. Solvent was evaporated. Purification by silica gel chromatography (Eluent: 30% EtOAc in heptane) afforded the desired product (C60) (3R,7aS)-3-phenyl-3,6,7,7a-tetrahydro-1H-pyrrolo[1,2-c]oxazol-5-one (4.8 g, 48%). ¹H NMR (400 MHz, Chloroform-d) δ: 7.43 (d, J=7 Hz, 2H), 7.37-7.29 (m, 3H), 6.32 (s, 1H), 4.24-4.17 (m, 1H), 4.15-4.11 (m, 1H), 3.48 (t, J=8.04 Hz, 1H), 2.85-2.76 (m, 1H), 2.59-2.51 (m, 1H), 2.42-2.33 (m, 1H), 1.98-1.91 (m, 1H). LCMS m/z 204.0 [M+H]⁺.

### Step 5. Synthesis of tert-butyl N-[(3R,6S,7aS)-5-oxo-3-phenyl-3,6,7,7a-tetrahydro-1H-pyrrolo[1,2-c]oxazol-6-yl]carbamate (C61)

LDA (2.9522 mL of 2 M, 5.9045 mmol) was cooled to −78° C. then (3R,7aS)-3-phenyl-3,6,7,7a-tetrahydro-1H-pyrrolo[1,2-c]oxazol-5-one (C60) (1 g, 4.9204 mmol) in THF (5 mL) was added. Reaction was stirred at −78° C. for 30 minutes. DPPA (2.7082 g, 2.1158 mL, 9.8408 mmol) was added and the reaction mixture was stirred for 10 minutes, then Boc anhydride (2.1477 g, 2.2607 mL, 9.8408 mmol) was added and the reaction was stirred for 17 hours. Reaction mixture was partitioned between ethyl acetate (2×125 mL) and brine (2×200 mL). Organic layer was dried over anhydrous magnesium sulfate and evaporated through reduced pressure. Purification by silica gel chromatography (Eluent: 20% EtOAc in heptane) afforded the desired product (C61) tert-butyl N-[(3R,6S,7aS)-5-oxo-3-phenyl-3,6,7,7a-tetrahydro-1H-pyrrolo[1,2-c]oxazol-6-yl]-carbamate (522 mg, 33%)¹H NMR (400 MHz, Chloroform-d) δ 7.46-7.28 (m, 5H), 5.20 (d, J=6.5 Hz, 1H), 4.62 (s, 1H), 4.25 (dd, J=8.4, 6.3 Hz, 1H), 4.11-4.00 (m, 1H), 3.62 (dd, J=8.4, 6.9 Hz, 1H), 3.00 (d, J=13.0 Hz, 1H), 1.75 (q, J=11.6 Hz, 1H), 1.45 (s, 9H). LCMS m/z 319.0 [M+H]⁺.

### Step 6. Synthesis of (3S,5S)-3-amino-5-(hydroxymethyl)pyrrolidin-2-one (S18)

A solution of tert-butyl N-[(3R,6S,7aS)-5-oxo-3-phenyl-3,6,7,7a-tetrahydro-1H-pyrrolo[1,2-c]oxazol-6-yl]carbamate (C61) (1.5 g, 4.7115 mmol) in DCM (15 mL) was cooled to 0° C., then TFA (11.100 g, 7.5 mL, 97.349 mmol) was added. The reaction mixture was stirred at room temperature for 2 hours. Solvent was removed in vacuo. 1,4-Dioxane-HCl (4 M) was added and reaction was stirred for 30 minutes. Solvent was removed to give (S18) (3S,5S)-3-amino-5-(hydroxymethyl)pyrrolidin-2-one (Hydrochloric Acid) (750 mg, 96%)¹H NMR (400 MHz, DMSO-d₆) δ 8.58 (m, 4H), 4.40 (m, 1H), 3.93 (m, 1H), 3.56 (m, 1H), 3.42 (m, 2H), 2.38 (m, 1H), 1.68 (m, 1H). LCMS m/z 131.0 [M+H]⁺.

### Preparation of S19

### 2-[5,7-difluoro-2-(4-fluorophenyl)-1H-indol-3-yl]ethanamine (S19)

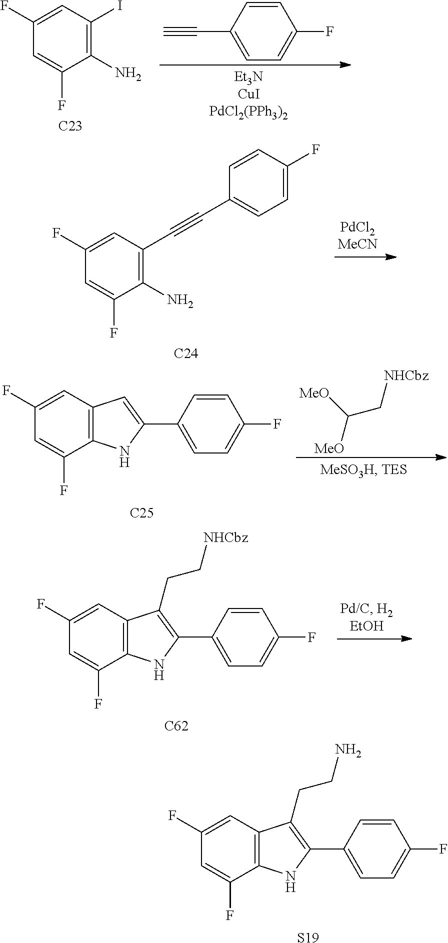

### Step 1. Synthesis of 2,4-difluoro-6-[2-(4-fluorophenyl)ethynyl]aniline (C24)

To a flask containing 2,4-difluoro-6-iodo-aniline C23 (134 g, 525.5 mmol) was added NEt₃ (1.3 L), followed by DMF (250 mL), 1-ethynyl-4-fluoro-benzene (83.5 g, 695.1 mmol), CuI (20.5 g, 107.6 mmol), and PdCl₂(PPh₃)₂ (25 g, 35.6 mmol). The mixture was allowed to stir at room temperature for 2 hours. Solvent was removed under reduced pressure and water (500 mL) was added. The mixture was extracted with Ethyl acetate, filtered and concentrated in vacuo. The product mixture was filtered through a silica gel plug (Eluent: CH₂Cl₂), followed by a second silica plug filtration (Eluent: 30-40% EtOAc in Heptane). The resulting crude was purified via silica gel chromatography (Gradient: 0-20% EtOAc in heptane) to afford the product 2,4-difluoro-6-[2-(4-fluorophenyl)ethynyl]aniline (C24) (87 g, 60%) as a pale yellow solid. ¹H NMR (300 MHz, Chloroform-d) δ 7.58-7.45 (m, 2H), 7.14-7.02 (m, 2H), 6.92 (ddd, J=8.8, 2.8, 1.7 Hz, 1H), 6.87-6.71 (m, 1H), 4.15 (s, 2H). LCMS m/z 248.0 [M+H]⁺.

### Step 2. Synthesis of 5,7-difluoro-2-(4-fluorophenyl)-1H-indole (C25)

To a solution of 2,4-difluoro-6-[2-(4-fluorophenyl)ethynyl]aniline C24 (46 g, 167.5 mmol) in DMF (600 mL) was added CuI (1.9 g, 10.0 mmol) and the reaction was heated at reflux. Water (800 mL) was added and the mixture extracted with MTBE. The mixture was then washed with sat. NaCl solution, dried over Na₂SO₄ and then concentrated in vacuo to afford the product 5,7-difluoro-2-(4-fluorophenyl)-1H-indole C25, which was used in subsequent steps without further purification (41 g, 87%). ¹H NMR (300 MHz, Chloroform-d) δ 8.43 (s, 1H), 7.72-7.58 (m, 2H), 7.27-7.15 (m, 2H), 7.09 (dd, J=9.0, 2.1 Hz, 1H), 6.85-6.63 (m, 2H). LCMS m/z 248.0 [M+H]⁺.

### Step 3. Synthesis of benzyl N-[2-[5,7-difluoro-2-(4-fluorophenyl)-1H-indol-3-yl]ethyl]carbamate (C62)

To a solution of 5,7-difluoro-2-(4-fluorophenyl)-1H-indole C25 (5.43 g, 22.0 mmol) in DCM (50 mL) was added methanesulfonic acid (4.5 mL, 69.4 mmol). After stirring for 20 minutes, benzyl N-(2,2-dimethoxyethyl)carbamate (5.5 g, 23.0 mmol) and triethylsilane (10.5 mL, 65.7 mmol) were added and the reaction was stirred overnight. The reaction was concentrated, and the crude was dissolved in DMSO and purified by reversed-phase chromatography (C18 column; Gradient: 0-100% MeCN in H₂O with 0.1% TFA) to afford the product benzyl N-[2-[5,7-difluoro-2-(4-fluorophenyl)-1H-indol-3-yl]ethyl]carbamate (C62) as a solid (9 g, 77%). LCMS m/z 425.2 [M+H]⁺.

### Step 4. Synthesis of 2-[5,7-difluoro-2-(4-fluorophenyl)-1H-indol-3-yl]ethanamine (S19)

To a solution of benzyl N-[2-[5,7-difluoro-2-(4-fluorophenyl)-1H-indol-3-yl]ethyl]carbamate C62 (5.3 g, 12.5 mmol) in EtOH (50 mL) was added Pd/C (200 g, 1.88 mol). The reaction was stirred under a H₂ atmosphere using a balloon overnight. The reaction was then filtered and concentrated. The crude was dissolved in DMSO and purified by reversed-phase chromatography (C18 column; Gradient: 0-100% MeCN in H₂O with 0.1% TFA) to afford the title compound as a TFA salt (S19) (2.5 g, 49%). ¹H NMR (300 MHz, Acetone-d₆) δ 10.90 (s, 1H), 7.95-7.64 (m, 2H), 7.44 (dd, J=9.4, 2.2 Hz, 1H), 7.39-7.25 (m, 2H), 6.86 (ddd, J=11.1, 9.6, 2.2 Hz, 1H), 4.32-3.91 (m, 2H), 3.56-3.36 (m, 2H), 2.85 (s, 2H). LCMS m/z 291.1 [M+H]⁺.

### Compound 1

### 3-[5-fluoro-2-(4-fluorophenyl)-1H-indol-3-yl]-N-[(1S)-2,2,2-trifluoro-1-(hydroxymethyl)ethyl]propanamide (1)

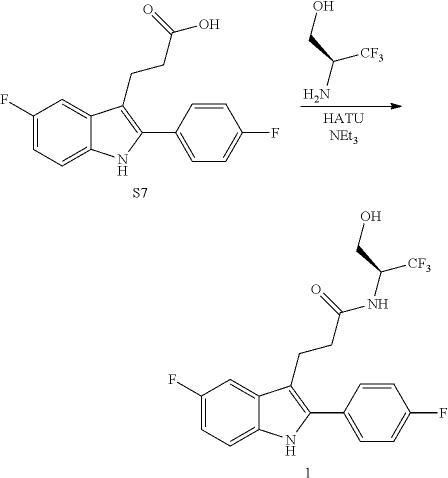

### Preparation of 3-[5-fluoro-2-(4-fluorophenyl)-1H-indol-3-yl]-N-[(1S)-2,2,2-trifluoro-1-(hydroxymethyl)ethyl]propanamide (1)

To a solution of (2S)-2-amino-3,3,3-trifluoro-propan-1-ol hydrochloride (16 mg, 0.097 mmol) in DMSO (1 mL) was added 3-[5-fluoro-2-(4-fluorophenyl)-1H-indol-3-yl]propanoic acid S7 (20 mg, 0.066 mmol), HATU (40 mg, 0.11 mmol), and NEt₃ (50 μL, 0.36 mmol). The mixture was allowed to stir at room temperature for 12 hours. The mixture was then purified by reversed-phase HPLC (Method: C18 Waters Sunfire column (30×150 mm, 5 micron). Gradient:

MeCN in H₂O with 0.1% trifluoroacetic acid) to afford the product (1) (21 mg, 74%). ¹H NMR (300 MHz, Methanol-d₄) δ 7.71-7.55 (m, 2H), 7.42-7.04 (m, 4H), 6.97-6.68 (m, 1H), 4.70-4.56 (m, 1H), 3.75 (dd, J=11.8, 4.8 Hz, 1H), 3.64 (dd, J=11.8, 6.7 Hz, 1H), 3.21-3.08 (m, 2H), 2.72-2.51 (m, 2H). LCMS m/z 413.1 [M+H]⁺.

### Compound 2

### 3-[5-fluoro-2-(4-fluorophenyl)-1H-indol-3-yl]-N-[(3S,4S)-4-hydroxypyrrolidin-3-yl]propanamide hydrochloride (2)

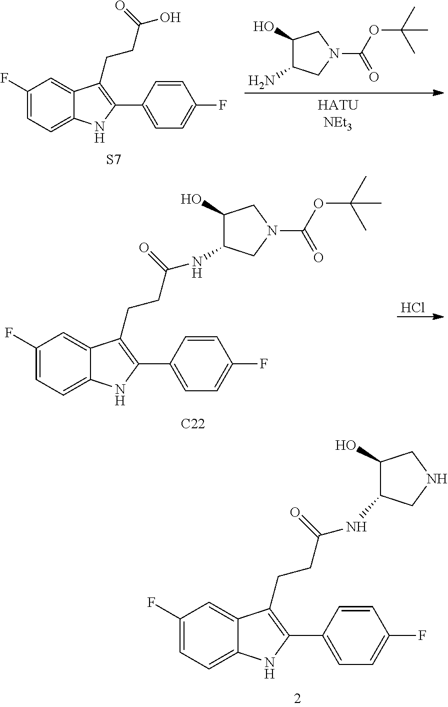

### Step 1. Synthesis of tert-butyl (3S,4S)-3-[3-[5-fluoro-2-(4-fluorophenyl)-1H-indol-3-yl]propanoylamino]-4-hydroxy-pyrrolidine-1-carboxylate (C22)

To a solution of tert-butyl (3S,4S)-3-amino-4-hydroxy-pyrrolidine-1-carboxylate C21 (20 mg, 0.099 mmol) in DMSO (1 mL) was added 3-[5-fluoro-2-(4-fluorophenyl)-1H-indol-3-yl]propanoic acid S7 (20 mg, 0.066 mmol), HATU (40 mg, 0.11 mmol), and NEt₃ (50 μL, 0.36 mmol). The mixture was allowed to stir at room temperature for 12 hours. The mixture was then purified by reversed-phase HPLC (Method: C18 Waters Sunfire column (30×150 mm, 5 micron). Gradient: MeCN in H₂O with 0.1% trifluoroacetic acid) to afford the product (25 mg, 75%). ¹H NMR (400 MHz, Methanol-d₄) δ 7.64 (ddd, J=8.8, 5.2, 2.2 Hz, 2H), 7.30 (td, J=6.2, 5.7, 2.9 Hz, 2H), 7.25-7.18 (m, 2H), 6.87 (tt, J=8.8, 2.2 Hz, 1H), 4.02 (ddt, J=11.1, 5.5, 2.3 Hz, 1H), 3.89 (ddt, J=9.0, 4.4, 2.4 Hz, 1H), 3.55 (ddd, J=11.5, 5.9, 4.2 Hz, 1H), 3.45 (m, 1H), 3.25-3.08 (m, 4H), 2.60-2.43 (m, 2H), 1.43 (d, J=1.0 Hz, 9H). LCMS m/z 486.2 [M+H]⁺.

### Step 2. Synthesis of 3-[5-fluoro-2-(4-fluorophenyl)-1H-indol-3-yl]-N-[(3S,4S)-4-hydroxypyrrolidin-3-yl]propanamide hydrochloride (2)

To a solution of tert-butyl (3S,4S)-3-[3-[5-fluoro-2-(4-fluorophenyl)-1H-indol-3-yl]propanoylamino]-4-hydroxy-pyrrolidine-1-carboxylate C22 (25.3 mg, 0.0501 mmol) in methanol (1 mL) was added HCl in 1,4-dioxane (0.5 mL of 4 M, 2 mmol). The mixture was stirred at 50° C. for 20 minutes. The reaction mixture was concentrated under reduced pressure, and the residue was triturated with diethyl ether and n-heptane. Lyophilization afforded the product (2) (22 mg, 94%). ¹H NMR (400 MHz, Methanol-d₄) δ 7.65 (dd, J=8.5, 5.2 Hz, 2H), 7.36-7.16 (m, 4H), 6.88 (td, J=9.1, 2.3 Hz, 1H), 4.17 (s, 1H), 4.15-4.06 (m, 1H), 3.63-3.55 (m, 1H), 3.20-3.13 (m, 4H), 2.64-2.47 (m, 2H). LCMS m/z 486.2 [M+H]⁺.

### Compounds 3-90

Compounds 3-90 (see Table 2) were prepared from intermediate S7 using the appropriate reagent and using the amide formation methods as described for compounds 1-2. Amines were prepared by methods described above or obtained from commercial sources. Any modifications to methods are noted in Table 2 and accompanying footnotes.

TABLE 2
Method of preparation, structure and physicochemical data for compounds 3-90

| | | Amine | ¹H NMR; LCMS m/z |
|---|---|---|---|
| Compound | Method/Product | Reagent | [M + H]⁺ |
| 3 | As for Compound 1 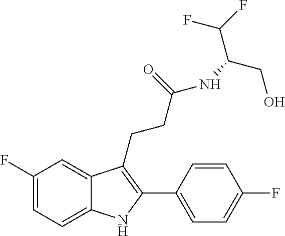 [SMILES: O=C(CCc1c(-c2ccc(F)cc2)[nH]c2ccc(F)cc12)N[C@@H](CO)C(F)F] | 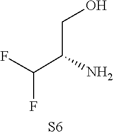 | ¹H NMR (300 MHz, Methanol-d₄) δ 7.78-7.56 (m, 2H), 7.36-7.09 (m, 4H), 6.87 (ddd, J = 9.4, 8.8, 2.5 Hz, 1H), 5.87 (td, J = 55.6, 3.3 Hz, 1H), 4.24 (tdq, J = 12.1, 5.9, 3.4, 3.0 Hz, 1H), 3.68-3.53 (m, 2H), 3.22- 3.03 (m, 2H), 2.72-2.52 (m, 2H); LCMS m/z 395.12 [M + H]⁺. |
| 4 | As for Compound 1 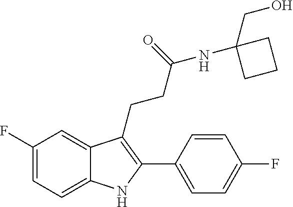 [SMILES: O=C(CCc1c(-c2ccc(F)cc2)[nH]c2ccc(F)cc12)NC1(CO)CCC1] | 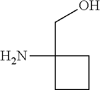 | ¹H NMR (300 MHz, Chloroform-d) δ 8.14 (s, 1H), 7.60-7.47 (m, 2H), 7.41- 7.30 (m, 2H), 7.24-7.13 (m, 2H), 6.99 (td, J = 9.0, 2.5 Hz, 1H), 5.60 (s, 1H), 3.93 (s, 1H), 3.66 (s, 2H), 3.20 (dd, J = 8.1, 7.0 Hz, 2H), 2.54 (dd, J = 8.2, 6.8 Hz, 2H), 2.14 (tq, J = 6.2, 3.2, 2.7 Hz, 2H), 1.91- 1.68 (m, 4H); LCMS m/z 385.09 [M + H]⁺. |
| 5 | As for Compound 2 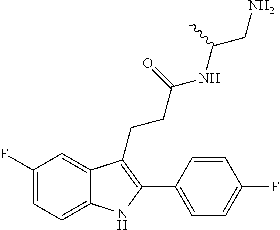 [SMILES: CC(CN)NC(=O)CCc1c(-c2ccc(F)cc2)[nH]c2ccc(F)cc12] | 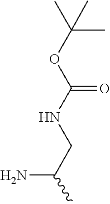 | ¹H NMR (300 MHz, Methanol-d₄) δ 7.69-7.55 (m, 2H), 7.37-7.16 (m, 4H), 6.88 (ddd, J = 9.4, 8.8, 2.5 1H), 3.74 (ddd, J = 5.9, 4.9, 1.3 Hz, 1H), 3.17 (dd, J = 8.2, 7.3 Hz, 2H), 2.96 (dd, J = 12.9, 4.4 Hz, 1H), 2.82 (dd, J = 12.9, 8.9 Hz, 1H), 2.63- 2.51 (m, 2H), 1.05 (d, J = 6.9 Hz, 3H); LCMS m/z 358.21 [M + H]⁺. |
| 6 | As for Compound 1 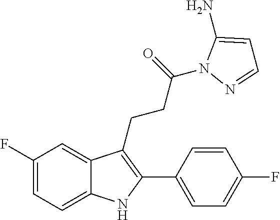 [SMILES: Nc1ccnn1C(=O)CCc1c(-c2ccc(F)cc2)[nH]c2ccc(F)cc12] | 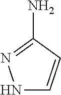 | LCMS m/z 366.96 [M + H]⁺. |
| 7 | As for Compound 2 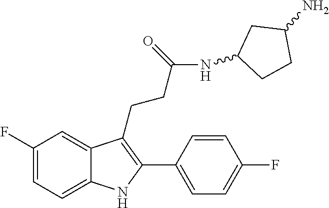 [SMILES: NC1CCC(NC(=O)CCc2c(-c3ccc(F)cc3)[nH]c3ccc(F)cc23)C1] | 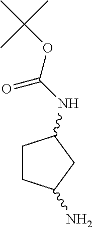 | LCMS m/z 384.14 [M + H]⁺. |
| 8 | As for Compound 1 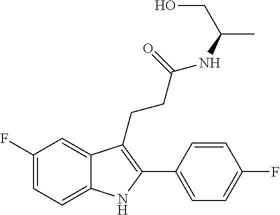 [SMILES: C[C@H](CO)NC(=O)CCc1c(-c2ccc(F)cc2)[nH]c2ccc(F)cc12] | 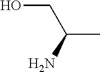 | 1H NMR (300 MHz, Chloroform-d) δ 8.06 (s, 1H), 7.64-7.47 (m, 2H), 7.41- 7.30 (m, 1H), 7.26-7.17 (m, 2H), 6.98 (td, J = 9.0, 2.5 Hz, 1H), 5.39 (d, J = 7.2 Hz, 1H), 3.97 (ddt, J = 10.4, 6.9, 3.5 Hz, 1H), 3.54 (dd, J = 11.0, 3.5 Hz, 1H), 3.40 (dd, J = 11.0, 6.0 Hz, 1H), 3.21 (dd, J = 8.1, 6.7 Hz, 2H), 2.55 (t, J = 7.6 Hz, 2H), 1.02 (d, J = 6.9 Hz, 3H); LCMS m/z 359.1 [M + H]⁺. |
| 9 | As for Compound 1 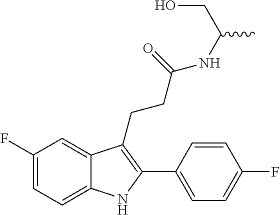 [SMILES: CC(CO)NC(=O)CCc1c(-c2ccc(F)cc2)[nH]c2ccc(F)cc12] | 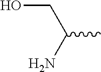 | LCMS m/z 359 [M + H]⁺. |
| 10 | As for Compound 2 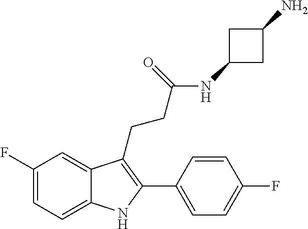 [SMILES: N[C@H]1C[C@@H](NC(=O)CCc2c(-c3ccc(F)cc3)[nH]c3ccc(F)cc23)C1] | 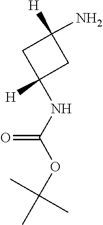 | LCMS m/z 370.32 [M + H]⁺. |
| 11 | As for Compound 1 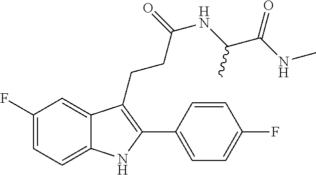 [SMILES: CNC(=O)C(C)NC(=O)CCc1c(-c2ccc(F)cc2)[nH]c2ccc(F)cc12] | 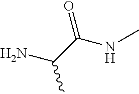 | ¹H NMR (300 MHz, Methanol-d₄) δ 7.74-7.59 (m, 2H), 7.39-7.26 (m, 2H), 7.26-7.14 (m, 2H), 6.87 (ddd, J = 9.4, 8.8, 2.5 Hz, 1H), 4.23 (q, J = 7.2 Hz, 1H), 3.20-3.06 (m, 2H), 2.66 (s, 3H), 2.58 (td, J = 7.5, 3.0 Hz, 2H), 1.19 (d, J = 7.2 Hz, 3H); LCMS m/z 386.1 [M + H]⁺. |
| 12 | As for Compound 1 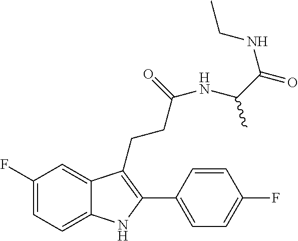 [SMILES: CCNC(=O)C(C)NC(=O)CCc1c(-c2ccc(F)cc2)[nH]c2ccc(F)cc12] | 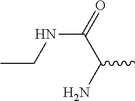 | ¹H NMR (400 MHz, Methanol-d₄) δ 7.69-7.58 (m, 2H), 7.37-7.26 (m, 2H), 7.25-7.14 (m, 2H), 6.87 (ddd, J = 9.4, 8.6, 2.5 Hz, 1H), 4.23 (q, J = 7.2 Hz, 1H), 3.15 (dddd, J = 8.5, 7.2, 5.1, 1.4 Hz, 4H), 2.57 (td, J = 7.6, 3.9 Hz, 2H), 1.19 (d, J = 7.2 Hz, 3H), 1.06 (t, J = 7.3 Hz, 3H); LCMS m/z 400.18 [M + H]⁺. |
| 13 | As for Compound 1 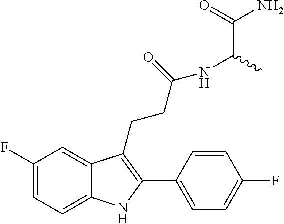 [SMILES: CC(NC(=O)CCc1c(-c2ccc(F)cc2)[nH]c2ccc(F)cc12)C(N)=O] | 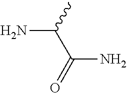 | ¹H NMR (300 MHz, Methanol-d₄) δ 10.68 (s, 1H), 8.06 (d, J = 7.7 Hz, 1H), 7.72- 7.54 (m, 2H), 7.42-7.03 (m, 4H), 6.87 (ddd, J = 9.4, 8.8, 2.5 Hz, 1H), 4.36-4.20 (m, 1H), 3.25-3.01 (m, 2H), 2.57 (td, J = 7.4, 2.0 Hz, 2H), 1.22 (d, J = 7.2 Hz, 3H); LCMS m/z 372.13 [M + H]⁺. |
| 14 | As for Compound 1 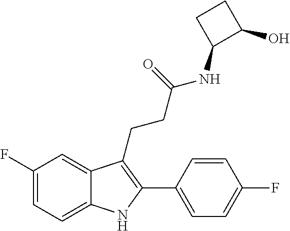 [SMILES: O=C(CCc1c(-c2ccc(F)cc2)[nH]c2ccc(F)cc12)N[C@H]1CC[C@H]1O] | 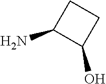 | ¹H NMR (400 MHz, Methanol-d₄) δ 7.72-7.59 (m, 2H), 7.36-7.28 (m, 2H), 7.28-7.10 (m, 2H), 6.87 (ddd, J = 9.4, 8.7, 2.5 Hz, 1H), 4.38-4.30 (m, 1H), 4.24 (td, J = 7.7, 4.0 Hz, 1H), 3.24- 3.06 (m, 2H), 2.62-2.53 (m, 2H), 2.17-1.96 (m, 2H), 1.91- 1.75 (m, 1H); LCMS m/z 371.16 [M + H]⁺. |
| 15 | As for Compound 1 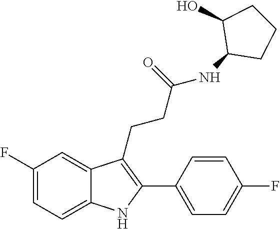 [SMILES: O=C(CCc1c(-c2ccc(F)cc2)[nH]c2ccc(F)cc12)N[C@@H]1CCC[C@@H]1O] | 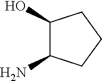 | LCMS m/z 385.29 [M + H]⁺. |
| 16 | As for Compound 1 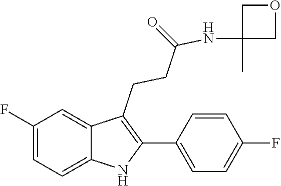 [SMILES: CC1(NC(=O)CCc2c(-c3ccc(F)cc3)[nH]c3ccc(F)cc23)COC1] | 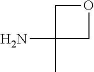 | LCMS m/z 371 [M + H]⁺. |
| 17 | As for Compound 1 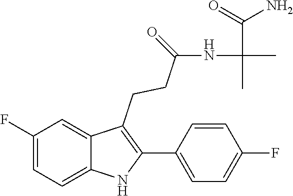 [SMILES: CC(C)(NC(=O)CCc1c(-c2ccc(F)cc2)[nH]c2ccc(F)cc12)C(N)=O] | 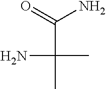 | ¹H NMR (300 MHz, Methanol-d₄) δ 7.73-7.52 (m, 2H), 7.41-7.12 (m, 4H), 6.87 (ddd, J = 9.5, 8.7, 2.5 Hz, 1H), 3.19-3.04 (m, 2H), 2.63-2.49 (m, 2H), 1.37 (s, 6H); LCMS m/z 386.07 [M + H]⁺. |
| 18 | As for Compound 1 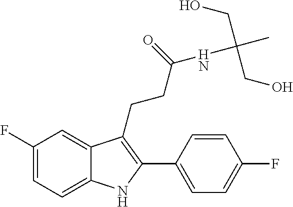 [SMILES: CC(CO)(CO)NC(=O)CCc1c(-c2ccc(F)cc2)[nH]c2ccc(F)cc12] | 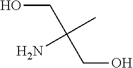 | ¹H NMR (400 MHz, Methanol-d₄) δ 7.73-7.51 (m, 2H), 7.39-7.28 (m, 2H), 7.26-7.13 (m, 2H), 6.87 (ddd, J = 9.5, 8.7, 2.5 Hz, 1H), 3.70-3.45 (m, 4H), 3.20- 3.07 (m, 2H), 2.58-2.48 (m, 2H), 1.13 (s, 3H); LCMS m/z 389.17 [M + H]⁺. |
| 19 | As for Compound 1 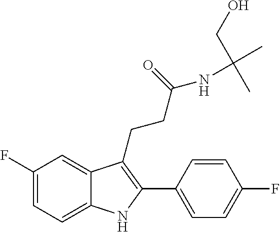 [SMILES: CC(C)(CO)NC(=O)CCc1c(-c2ccc(F)cc2)[nH]c2ccc(F)cc12] | 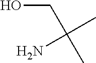 | ¹H NMR (300 MHz, Chloroform-d) δ 8.17 (s, 1H), 7.64-7.45 (m, 2H), 7.38- 7.28 (m, 2H), 7.24-7.09 (m, 2H), 6.98 (td, J = 9.0, 2.5 Hz, 1H), 5.29 (s, 1H), 3.45 (s, 2H), 3.19 (dd, J = 8.2, 6.9 Hz, 2H), 2.53 (dd, J = 8.1, 6.9 Hz, 2H), 1.09 (s, 6H); LCMS m/z 373.11 [M + H]⁺. |
| 20 | As for Compound 1 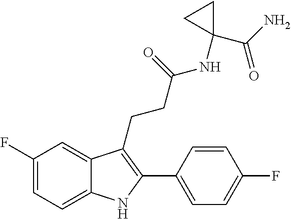 [SMILES: NC(=O)C1(NC(=O)CCc2c(-c3ccc(F)cc3)[nH]c3ccc(F)cc23)CC1] | 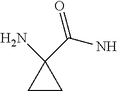 | ¹H NMR (400 MHz, Methanol-d₄) δ 7.72-7.60 (m, 2H), 7.36-7.27 (m, 2H), 7.27-7.14 (m, 2H), 6.88 (ddd, J = 9.4, 8.6, 2.5 Hz, 1H), 3.19-3.08 (m, 2H), 2.62- 2.52 (m, 2H), 1.39-1.25 (m, 2H), 0.83-0.67 (m, 2H); LCMS m/z 384.16 [M + H]⁺. |
| 21 | As for Compound 1 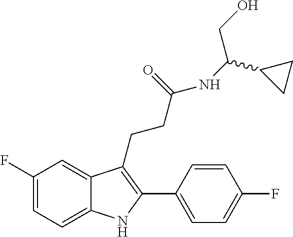 [SMILES: O=C(CCc1c(-c2ccc(F)cc2)[nH]c2ccc(F)cc12)NC(CO)C1CC1] | 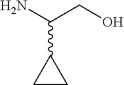 | ¹H NMR (300 MHz, Methanol-d₄) δ 7.73-7.60 (m, 2H), 7.42-7.13 (m, 4H), 6.87 (ddd, J = 9.4, 8.7, 2.5 Hz, 1H), 3.56 (qd, J = 11.1, 5.2 Hz, 2H), 3.28-3.03 (m, 3H), 2.56 (dd, J = 8.7, 7.0 Hz, 2H), 0.95-0.73 (m, 1H), 0.46 (tdd, J = 8.2, 5.1, 3.6 Hz, 1H), 0.32 (dddd, J = 9.5, 8.3, 4.8, 3.4 Hz, 1H), 0.26-0.05 (m, 2H); LCMS m/z 385.16 [M + H]⁺. |
| 22 | As for Compound 1 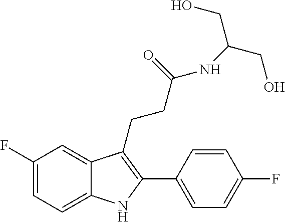 [SMILES: O=C(CCc1c(-c2ccc(F)cc2)[nH]c2ccc(F)cc12)NC(CO)CO] | 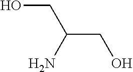 | ¹H NMR (300 MHz, Methanol-d₄) δ 7.69-7.55 (m, 2H), 7.38-7.12 (m, 4H), 6.87 (td, J = 9.1, 2.5 Hz, 1H), 3.88 (dq, J = 8.3, 5.5 Hz, 1H), 3.31 (p, J = 1.6 Hz, 4H), 3.23- 3.03 (m, 2H), 2.63-2.46 (m, 2H); LCMS m/z 375.09 [M + H]⁺. |
| 23 | As for Compound 1 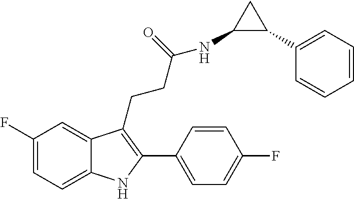 [SMILES: O=C(CCc1c(-c2ccc(F)cc2)[nH]c2ccc(F)cc12)N[C@H]1C[C@@H]1c1ccccc1] | 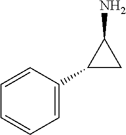 | LCMS m/z 417.01 [M + H]⁺. |
| 24 | As for Compound 1 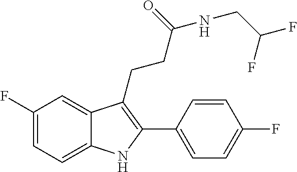 [SMILES: O=C(CCc1c(-c2ccc(F)cc2)[nH]c2ccc(F)cc12)NCC(F)F] | 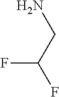 | LCMS m/z 365.29 [M + H]⁺. |
| 25 | As for Compound 1 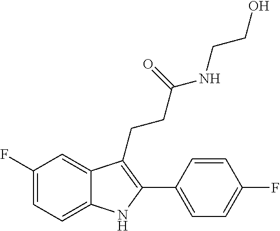 [SMILES: O=C(CCc1c(-c2ccc(F)cc2)[nH]c2ccc(F)cc12)NCCO] | 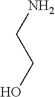 | LCMS m/z 345.28 [M + H]⁺. |
| 26 | As for Compound 1 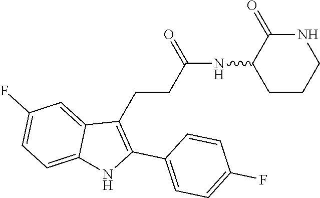 [SMILES: O=C(CCc1c(-c2ccc(F)cc2)[nH]c2ccc(F)cc12)NC1CCCNC1=O] | 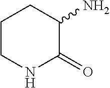 | ¹H NMR (300 MHz, Methanol-d₄) δ 7.68-7.54 (m, 2H), 7.39-7.11 (m, 4H), 6.87 (ddd, J = 9.4, 8.7, 2.5 Hz, 1H), 4.28 (dd, J = 10.7, 6.0 Hz, 1H), 3.26 (dd, J = 7.1, 5.0 Hz, 2H), 3.21-3.04 (m, 2H), 2.62-2.50 (m, 2H), 2.03- 1.90 (m, 1H), 1.83 (tq, J = 7.1, 3.7 Hz, 1H), 1.68-1.59 (m, 1H). One proton obscured by solvent peak. LCMS m/z 398.09 [M + H]⁺. |
| 27 | As for Compound 1 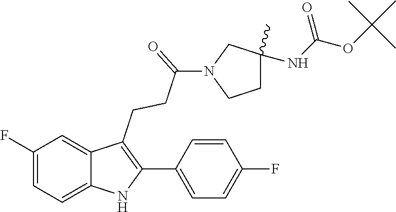 | 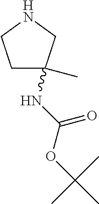 | ¹H NMR (300 MHz, Chloroform-d) δ 8.15 (2s,, 1H), 7.56 (ddt, J = 6.8, 5.3, 1.7 Hz, 2H), 7.29 (d, J = 2.3 Hz, 2H), 7.23-7.12 (m, 2H), 7.06-6.85 (m, 1H), 4.45 (s, 1H), 3.72-3.44 (m, 2H), 3.30 (d, J = 12.4 Hz, 1H), 3.19 (td, J = 7.2, 2.1 Hz, 3H), 2.57 (td, J = 9.9, 6.5 Hz, 2H), 2.18- 2.01 (m, 1H), 1.93-1.62 (m, 1H), 1.56-1.20 (m, 12H); LCMS m/z 484.03 [M + H]⁺. |
| 28 | As for Compound 1 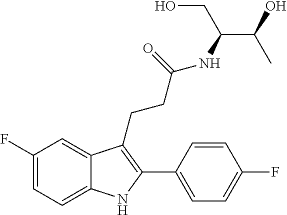 [SMILES: C[C@H](O)[C@H](CO)NC(=O)CCc1c(-c2ccc(F)cc2)[nH]c2ccc(F)cc12] | 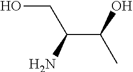 | ¹H NMR (400 MHz, Methanol-d₄) δ 7.76-7.55 (m, 2H), 7.40-7.27 (m, 2H), 7.27-7.13 (m, 2H), 6.87 (ddd, J = 9.5, 8.8, 2.5 Hz, 1H), 3.93 (qd, J = 6.4, 3.1 Hz, 1H), 3.78 (td, J = 6.3, 3.1 Hz, 1H), 3.56 (dd, J = 11.0, 6.3 Hz, 1H), 3.47 (dd, J = 11.0, 6.2 Hz, 1H), 3.16 (td, J = 7.3, 1.6 Hz, 2H), 2.72-2.52 (m, 2H), 0.99 (d, J = 6.5 Hz, 3H); LCMS m/z 389.17 [M + H]⁺. |
| 29 | As for Compound 1 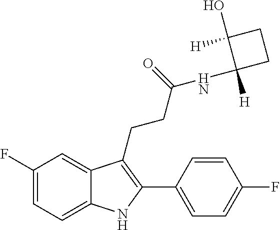 [SMILES: O=C(CCc1c(-c2ccc(F)cc2)[nH]c2ccc(F)cc12)N[C@H]1CC[C@@H]1O] | 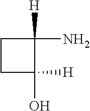 | ¹H NMR (400 MHz, Methanol-d₄) δ 7.69-7.58 (m, 2H), 7.36-7.26 (m, 2H), 7.26-7.12 (m, 2H), 6.94- 6.81 (m, 1H), 4.03 (dt, J = 9.7, 8.0 Hz, 1H), 3.87 (q, J = 7.9 Hz, 1H), 3.16-3.05 (m, 2H), 2.50 (ddd, J = 8.1, 6.6, 2.3 Hz, 2H), 1.94 (dddd, J = 10.7, 9.4, 8.0, 1.4 Hz, 1H), 1.49 (tt, J = 11.0, 9.0 Hz, 1H), 1.23-1.12 (m, 1H); LCMS m/z 371.16 [M + H]⁺. |
| 30 | As for Compound 1 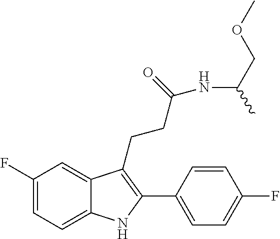 [SMILES: COCC(C)NC(=O)CCc1c(-c2ccc(F)cc2)[nH]c2ccc(F)cc12] | 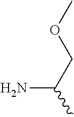 | ¹H NMR (300 MHz, Methanol-d₄) δ 7.71-7.57 (m, 2H), 7.37-7.11 (m, 4H), 6.87 (ddd, J = 9.4, 8.8, 2.5 Hz, 1H), 4.00 (dtd, J = 12.3, 6.8, 5.6 Hz, 1H), 3.25 (s, 3H), 3.25-3.19 (m, 1H), 3.17- 3.06 (m, 3H), 2.58-2.46 (m, 2H), 1.00 (d, J = 6.8 Hz, 3H); LCMS m/z 373.11 [M + H]⁺. |
| 31 | As for Compound 1 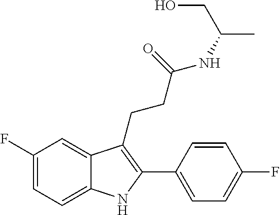 [SMILES: C[C@@H](CO)NC(=O)CCc1c(-c2ccc(F)cc2)[nH]c2ccc(F)cc12] | 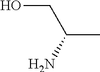 | ¹H NMR (300 MHz, Chloroform-d) δ 8.03 (s, 1H), 7.61-7.46 (m, 2H), 7.37- 7.30 (m, 1H), 7.25-7.09 (m, 2H), 6.99 (td, J = 9.0, 2.4 Hz, 1H), 5.37 (s, 1H), 3.98 (dp, J = 10.4, 3.4 Hz, 1H), 3.54 (dd, J = 11.0, 3.5 Hz, 1H), 3.40 (dd, J = 11.0, 6.0 Hz, 1H), 3.21 (dd, J = 8.3, 6.8 Hz, 2H), 2.55 (t, J = 7.6 Hz, 2H), 1.02 (d, J = 6.9 Hz, 3H); LCMS m/z 359.1 [M + H]⁺. |
| 32 | As for Compound 1 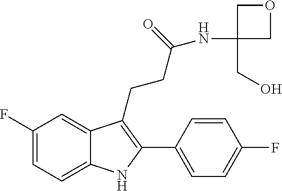 [SMILES: O=C(CCc1c(-c2ccc(F)cc2)[nH]c2ccc(F)cc12)NC1(CO)COC1] | 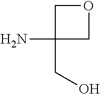 | ¹H NMR (300 MHz, Chloroform-d) δ 7.52 (s, 1H), 7.01 (m, 3H), 6.66 (m, 3H), 6.45 (s, 1H), 5.15 (s, 1H), 3.92 (d, J = 6.5 Hz, 2H), 3.82 (d, J = 7.0 Hz, 2H), 3.35 (s, 2H), 2.68 (s, 2H), 2.05 (d, J = 7.2 Hz, 2H); LCMS m/z 387.08 [M + H]⁺. |
| 33 | As for Compound 1 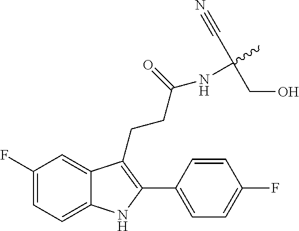 [SMILES: CC(C#N)(CO)NC(=O)CCc1c(-c2ccc(F)cc2)[nH]c2ccc(F)cc12] | 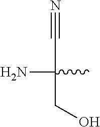 | ¹H NMR (300 MHz, Methanol-d₄) δ 7.72-7.52 (m, 2H), 7.36-7.14 (m, 4H), 6.87 (td, J = 9.2, 2.5 Hz, 1H), 3.78 (d, J = 11.0 Hz, 1H), 3.56 (d, J = 11.0 Hz, 1H), 3.21-3.06 (m, 2H), 2.61- 2.48 (m, 2H), 1.52 (s, 3H); LCMS m/z 384.15 [M + H]⁺. |
| 34 | As for Compound 1 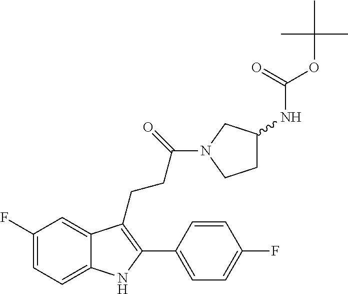 [SMILES: CC(C)(C)OC(=O)NC1CCN(C(=O)CCc2c(-c3ccc(F)cc3)[nH]c3ccc(F)cc23)C1] | 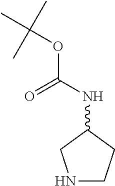 | LCMS m/z 470.04 [M + H]⁺. |
| 35 | As for Compound 1 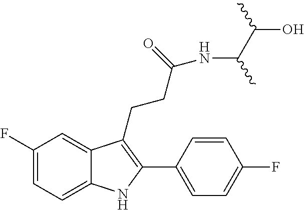 | 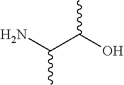 | ¹H NMR (300 MHz, Methanol-d₄) δ 7.73-7.59 (m, 2H), 7.41-7.13 (m, 4H), 6.87 (td, J = 9.1, 2.5 Hz, 1H), 3.85-3.68 (m, 1H), 3.67- 3.51 (m, 1H), 3.14 (ddd, J = 8.7, 6.7, 1.7 Hz, 2H), 2.61- 2.41 (m, 2H), 0.99 (ddd, J = 11.1, 10.1, 6.4 Hz, 6H); LCMS m/z 373.17 [M + H]⁺. |
| 36 | As for Compound 1 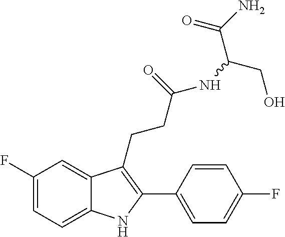 [SMILES: NC(=O)C(CO)NC(=O)CCc1c(-c2ccc(F)cc2)[nH]c2ccc(F)cc12] | 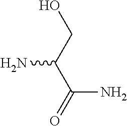 | ¹H NMR (300 MHz, Methanol-d₄) δ 7.72-7.54 (m, 2H), 7.35-7.27 (m, 2H), 7.27-7.08 (m, 2H), 6.87 (ddd, J = 9.4, 8.7, 2.5 Hz, 1H), 4.38 (t, J = 5.2 Hz, 1H), 3.73 (dd, J =11.1, 5.3 Hz, 1H), 3.65 (dd, J = 11.1, 5.1 Hz, 1H), 3.24-3.00 (m, 2H), 2.70-2.50 (m, 2H); LCMS m/z 388.05 [M + H]⁺. |
| 37 | As for Compound 2 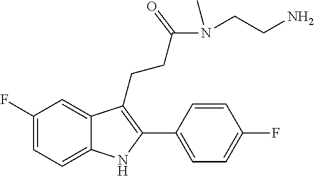 [SMILES: CN(CCN)C(=O)CCc1c(-c2ccc(F)cc2)[nH]c2ccc(F)cc12] | 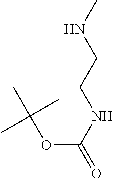 | LCMS m/z 358.39 [M + H]⁺. |
| 38 | As for Compound 1 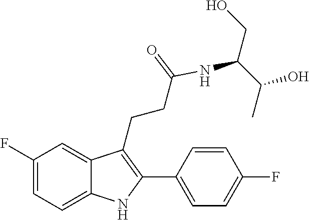 [SMILES: C[C@@H](O)[C@H](CO)NC(=O)CCc1c(-c2ccc(F)cc2)[nH]c2ccc(F)cc12] | 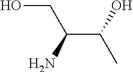 | ¹H NMR (400 MHz, Methanol-d₄) δ 7.72-7.54 (m, 2H), 7.40-7.16 (m, 4H), 6.87 (ddd, J = 9.4, 8.8, 2.5 Hz, 1H), 3.80-3.69 (m, 2H), 3.62 (d, J = 4.9 Hz, 2H), 3.22- 3.11 (m, 2H), 2.58 (dd, J = 8.6, 7.3 Hz, 2H), 1.03 (d, J = 6.1 Hz, 3H); LCMS m/z 389.17 [M + H]⁺. |
| 39 | As for Compound 1 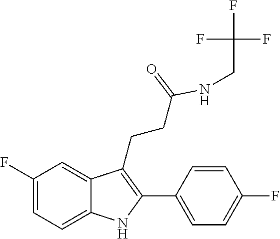 [SMILES: O=C(CCc1c(-c2ccc(F)cc2)[nH]c2ccc(F)cc12)NCC(F)(F)F] | 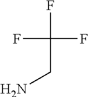 | LCMS m/z 383.22 [M + H]⁺. |
| 40 | As for Compound 1 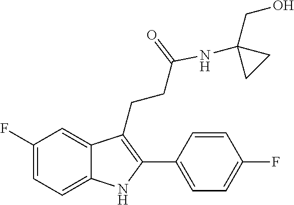 [SMILES: O=C(CCc1c(-c2ccc(F)cc2)[nH]c2ccc(F)cc12)NC1(CO)CC1] | 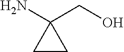 | ¹H NMR (300 MHz, Chloroform-d) δ 8.11 (s, 1H), 7.63-7.50 (m, 2H), 7.32 (dd, J = 8.8, 4.4 Hz, 1H), 7.29- 7.11 (m, 4H), 6.99 (td, J = 9.0, 2.5 Hz 1H) 5.76 (s 1H) 3.72 (s, 1H), 3.47 (s, 2H), 3.19 (dd, J = 8.2, 6.9 Hz, 2H), 2.52 (dd, J = 8.2, 6.9 Hz, 2H), 0.85-0.72 (m, 2H), 0.66- 0.45 (m, 2H); LCMS m/z 371.09 [M + H]⁺. |
| 41 | As for Compound 1 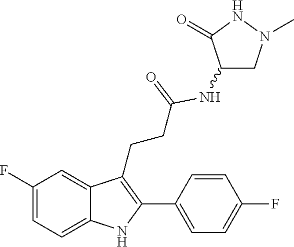 [SMILES: CN1CC(NC(=O)CCc2c(-c3ccc(F)cc3)[nH]c3ccc(F)cc23)C(=O)N1] | 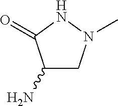 | ¹H NMR (300 MHz, Methanol-d₄) δ 10.69 (s, 1H), 7.75-7.55 (m, 2H), 7.41- 7.13 (m, 5H), 6.95-6.67 (m, 1H), 3.43 (s, 1H), 3.16 (t, J = 7.5 Hz, 2H), 2.62-2.52 (m, 5H); LCMS m/z 399.17 [M + H]⁺. |
| 42 | As for Compound 1 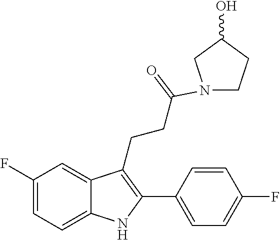 [SMILES: O=C(CCc1c(-c2ccc(F)cc2)[nH]c2ccc(F)cc12)N1CCC(O)C1] | 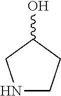 | LCMS m/z 371.27 [M + H]⁺. |
| 43 | As for Compound 1 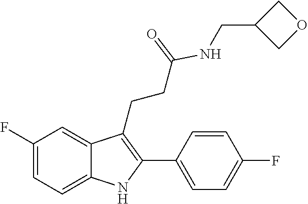 [SMILES: O=C(CCc1c(-c2ccc(F)cc2)[nH]c2ccc(F)cc12)NCC1COC1] | 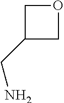 | LCMS m/z 371 [M + H]⁺. |
| 44 | As for Compound 1 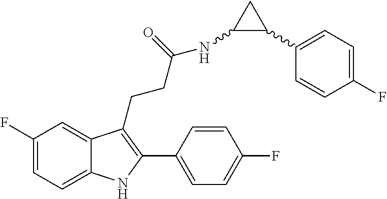 [SMILES: O=C(CCc1c(-c2ccc(F)cc2)[nH]c2ccc(F)cc12)NC1CC1c1ccc(F)cc1] | 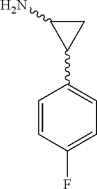 [SMILES: NC1CC1c1ccc(F)cc1] | LCMS m/z 435.01 [M + H]⁺. |
| 45 | As for Compound 1 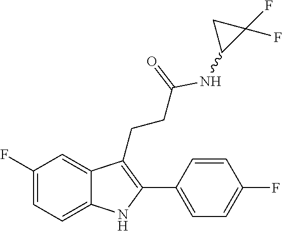 [SMILES: O=C(CCc1c(-c2ccc(F)cc2)[nH]c2ccc(F)cc12)NC1CC1(F)F] | 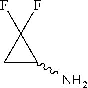 | LCMS m/z 377.2 [M + H]⁺. |
| 46 | As for Compound 1 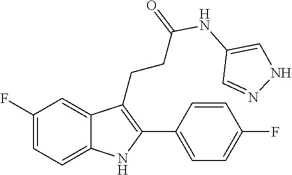 [SMILES: O=C(CCc1c(-c2ccc(F)cc2)[nH]c2ccc(F)cc12)Nc1cn[nH]c1] | 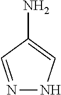 | LCMS m/z 366.96 [M + H]⁺. |
| 47 | As for Compound 1 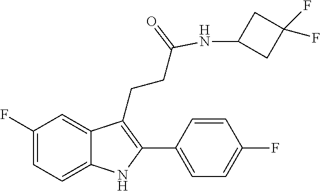 [SMILES: O=C(CCc1c(-c2ccc(F)cc2)[nH]c2ccc(F)cc12)NC1CC(F)(F)C1] | 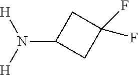 | LCMS m/z 391.24 [M + H]⁺. |
| 48 | As for Compound 1 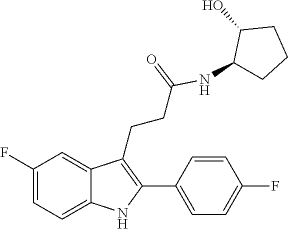 [SMILES: O=C(CCc1c(-c2ccc(F)cc2)[nH]c2ccc(F)cc12)N[C@@H]1CCC[C@H]1O] | 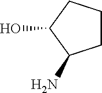 | LCMS m/z 385.16 [M + H]⁺. |
| 49 | As for Compound 2 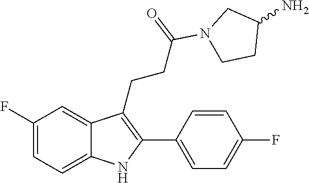 [SMILES: NC1CCN(C(=O)CCc2c(-c3ccc(F)cc3)[nH]c3ccc(F)cc23)C1] | 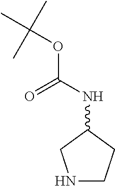 | LCMS m/z 370.23 [M + H]⁺. |
| 50 | As for Compound 1¹ 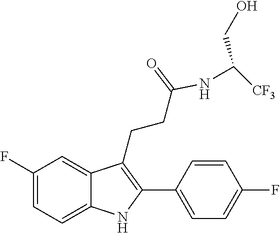 [SMILES: O=C(CCc1c(-c2ccc(F)cc2)[nH]c2ccc(F)cc12)N[C@H](CO)C(F)(F)F] | 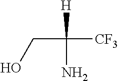 | ¹H NMR (300 MHz, Methanol-d₄) δ 6.22-6.07 (m, 2H), 5.87-5.75 (m, 2H), 5.75-5.62 (m, 2H), 5.34 (ddd, J = 9.4, 8.8, 2.5 Hz, 1H), 3.08 (ddt, J = 8.1, 6.7, 4.1 Hz, 0H), 2.22 (dd, J = 11.8, 4.8 Hz, 1H), 2.11 (dd, J = 11.8, 6.7 Hz, 1H), 1.70- 1.56 (m, 2H), 1.09 (ddd, J = 9.0, 6.3, 1.2 Hz, 2H); LCMS m/z 413.13 [M + H]⁺. |
| 51 | As for Compound 1 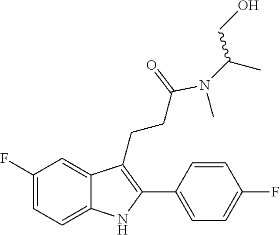 [SMILES: CC(CO)N(C)C(=O)CCc1c(-c2ccc(F)cc2)[nH]c2ccc(F)cc12] | 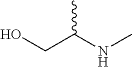 | ¹H NMR (300 MHz, Methanol-d₄) δ 7.75-7.53 (m, 2H), 7.40-7.12 (m, 4H), 6.88 (dddd, J = 9.6, 8.8, 2.5, 1.0 Hz, 1H), 4.72-3.74 (m, 1H), 3.55-3.33 (m, 2H), 3.16 (t, J = 7.9 Hz, 2H), 2.96- 2.80 (m, 1H), 2.72 (d, J = 1.7 Hz, 3H), 2.69-2.57 (m, 1H), 0.87 (dd, J = 53.0, 6.8 Hz, 3H); LCMS m/z 373.17 [M + H]⁺. |
| 52 | As for Compound 1 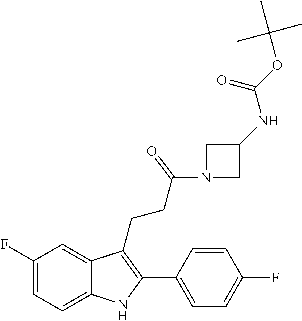 [SMILES: CC(C)(C)OC(=O)NC1CN(C(=O)CCc2c(-c3ccc(F)cc3)[nH]c3ccc(F)cc23)C1] | 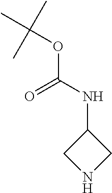 | LCMS m/z 456.01 [M + H]⁺. |
| 53 | As for Compound 2 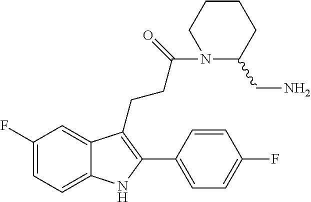 [SMILES: NCC1CCCCN1C(=O)CCc1c(-c2ccc(F)cc2)[nH]c2ccc(F)cc12] | 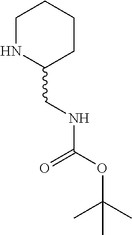 | LCMS m/z 398.27 [M + H]⁺. |
| 54 | As for Compound 2 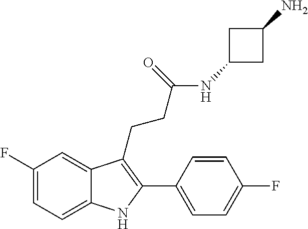 [SMILES: N[C@H]1C[C@H](NC(=O)CCc2c(-c3ccc(F)cc3)[nH]c3ccc(F)cc23)C1] | 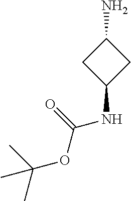 | ¹H NMR (300 MHz, Methanol-d₄) δ 7.73-7.58 (m, 2H), 7.38-7.16 (m, 4H), 6.87 (td, J = 9.1, 2.5 Hz, 1H), 4.40 (td, J = 8.3, 4.4 Hz, 1H), 3.86-3.68 (m, 2H), 3.61- 3.53 (m, 1H), 3.14 (t, J = 7.5 Hz, 2H), 2.51 (t, J = 7.5 Hz, 2H), 2.40 (tm, 2H), 2.27 (m, 2H); LCMS m/z 370.23 [M + H]⁺. |
| 55 | As for Compound 1 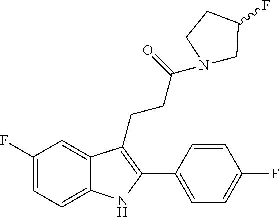 [SMILES: O=C(CCc1c(-c2ccc(F)cc2)[nH]c2ccc(F)cc12)N1CCC(F)C1] | 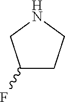 | LCMS m/z 372.83 [M + H]⁺. |
| 56 | As for Compound 2 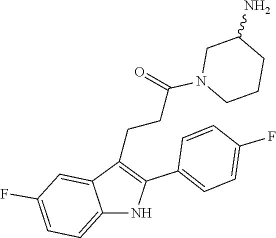 [SMILES: NC1CCCN(C(=O)CCc2c(-c3ccc(F)cc3)[nH]c3ccc(F)cc23)C1] | 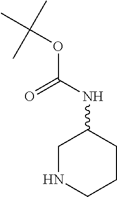 | LCMS m/z 384.09 [M + H]⁺. |
| 57 | As for Compound 1 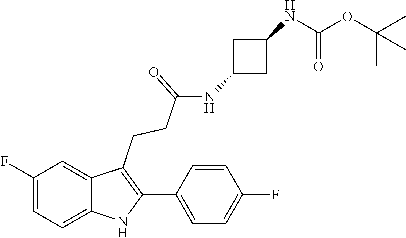 [SMILES: CC(C)(C)OC(=O)N[C@H]1C[C@H](NC(=O)CCc2c(-c3ccc(F)cc3)[nH]c3ccc(F)cc23)C1] | 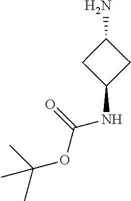 | LCMS m/z 470.1 [M + H]⁺. |
| 58 | As for Compound 1 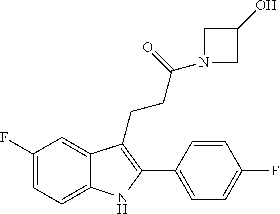 [SMILES: O=C(CCc1c(-c2ccc(F)cc2)[nH]c2ccc(F)cc12)N1CC(O)C1] | 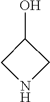 | LCMS m/z 357.26 [M + H]⁺. |
| 59 | As for Compound 1 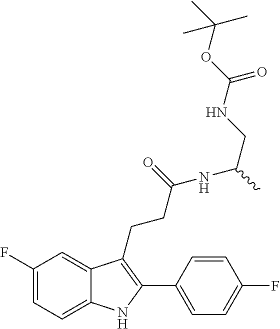 [SMILES: CC(CNC(=O)OC(C)(C)C)NC(=O)CCc1c(-c2ccc(F)cc2)[nH]c2ccc(F)cc12] | 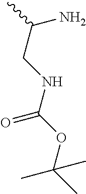 | ¹H NMR (300 MHz, Methanol-d₄) δ 7.74 (d, J = 8.1 Hz, 1H), 7.69-7.57 (m, 2H), 7.36-7.25 (m, 2H), 7.24 (d, J = 8.8 Hz, 1H), 6.87 (ddd, J = 9.5, 8.8, 2.5 Hz, 1H), 6.45 (s, 1H), 3.90 (p, J = 6.7 Hz, 1H), 3.13 (t, J = 7.9 Hz, 2H), 3.00 (q, J = 7.1, 6.5 Hz, 2H), 2.50 (td, J = 7.5, 3.1 Hz, 2H), 1.39 (s, 9H), 0.98 (d, J = 6.8 Hz, 3H); LCMS m/z 458.04 [M + H]⁺. |
| 60 | As for Compound 1 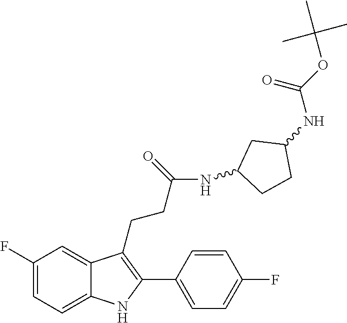 [SMILES: CC(C)(C)OC(=O)NC1CCC(NC(=O)CCc2c(-c3ccc(F)cc3)[nH]c3ccc(F)cc23)C1] | 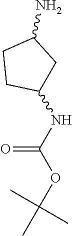 | LCMS m/z 484.1 [M + H]⁺. |
| 61 | As for Compound 1 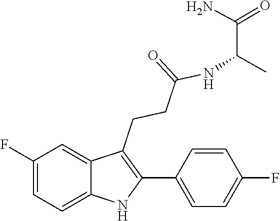 [SMILES: C[C@H](NC(=O)CCc1c(-c2ccc(F)cc2)[nH]c2ccc(F)cc12)C(N)=O] | 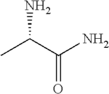 | ¹H NMR (300 MHz, Methanol-d₄) δ 7.76-7.51 (m, 2H), 7.43-7.05 (m, 4H), 6.87 (td, J = 9.1, 2.5 Hz, 1H), 4.28 (qt, J = 7.2, 3.5 Hz, 1H), 3.14 (dd, J = 8.7, 7.1 Hz, 2H), 2.57 (td, J = 7.4, 2.0 Hz, 2H), 1.23 (d, J = 7.2 Hz, 3H); LCMS m/z 372.19 [M + H]⁺; |
| 62 | As for Compound 1 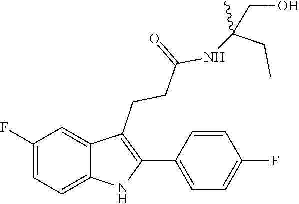 [SMILES: CCC(C)(CO)NC(=O)CCc1c(-c2ccc(F)cc2)[nH]c2ccc(F)cc12] | 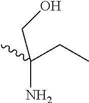 | ¹H NMR (400 MHz, Methanol-d₄) δ 7.71-7.51 (m, 2H), 7.42-7.09 (m, 4H), 6.87 (ddd, J = 9.4, 8.7, 2.5 Hz, 1H), 3.59 (d, J = 11.2 Hz, 1H), 3.47 (d, J = 11.2 Hz, 1H), 3.20-3.06 (m, 2H), 2.61- 2.42 (m, 2H), 1.76 (dq, J = 13.7, 7.5 Hz, 1H), 1.58-1.44 (m, 1H), 1.10 (s, 3H), 0.71 (t, J = 7.5 Hz, 3H); LCMS m/z 387.19 [M + H]⁺. |
| 63 | As for Compound 1 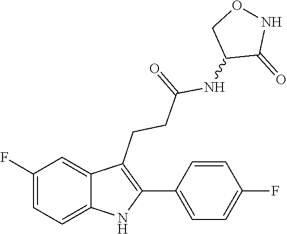 [SMILES: O=C(CCc1c(-c2ccc(F)cc2)[nH]c2ccc(F)cc12)NC1CONC1=O] | 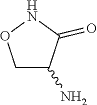 | LCMS m/z 386.14 [M + H]⁺. |
| 64 | As for Compound 1 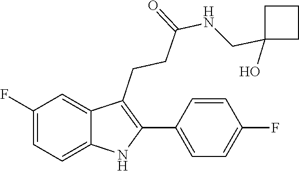 [SMILES: O=C(CCc1c(-c2ccc(F)cc2)[nH]c2ccc(F)cc12)NCC1(O)CCC1] | 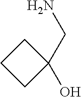 | LCMS m/z 385.3 [M + H]⁺. |
| 65 | As for Compound 1 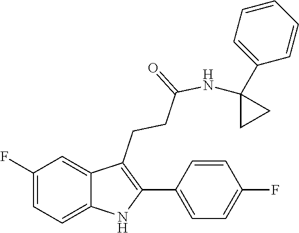 [SMILES: O=C(CCc1c(-c2ccc(F)cc2)[nH]c2ccc(F)cc12)NC1(c2ccccc2)CC1] | 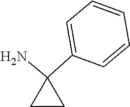 | ¹H NMR (300 MHz, Methanol-d₄) δ 7.73-7.58 (m, 2H), 7.37-7.28 (m, 2H), 7.24-7.12 (m, 4H), 7.12- 7.05 (m, 1H) 7.02-6.93 (m, 2H), 6.92-6.79 (m, 1H), 3.17 (t, J = 7.5 Hz, 2H), 2.59 (dd, J = 8.1, 7.0 Hz, 2H), 1.20- 0.95 (m, 4H); LCMS m/z 417.17 [M + H]⁺. |
| 66 | As for Compound 1 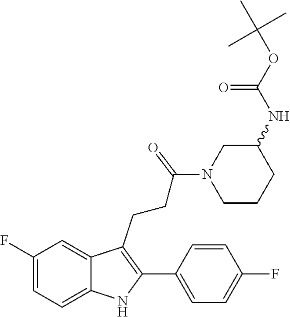 [SMILES: CC(C)(C)OC(=O)NC1CCCN(C(=O)CCc2c(-c3ccc(F)cc3)[nH]c3ccc(F)cc23)C1] | 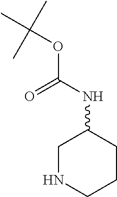 | LCMS m/z 483.87 [M + H]⁺. |
| 67 | As for Compound 2 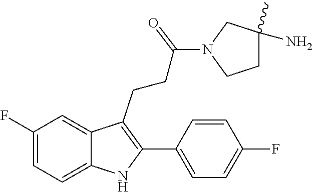 [SMILES: CC1(N)CCN(C(=O)CCc2c(-c3ccc(F)cc3)[nH]c3ccc(F)cc23)C1] | 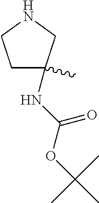 | LCMS m/z 384.23 [M + H]⁺. |
| 68 | As for Compound 1 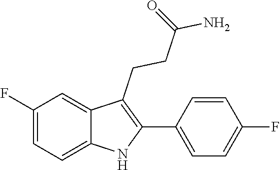 [SMILES: NC(=O)CCc1c(-c2ccc(F)cc2)[nH]c2ccc(F)cc12] | NH₃ | LCMS m/z 301.19 [M + H]⁺. |
| 69 | As for Compound 1 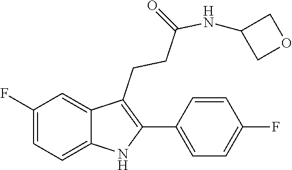 [SMILES: O=C(CCc1c(-c2ccc(F)cc2)[nH]c2ccc(F)cc12)NC1COC1] | 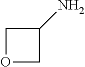 | LCMS m/z 356.97 [M + H]⁺. |
| 70 | As for Compound 1 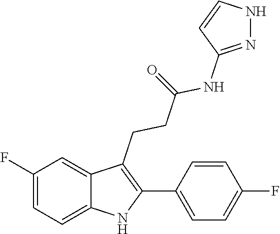 [SMILES: O=C(CCc1c(-c2ccc(F)cc2)[nH]c2ccc(F)cc12)Nc1cc[nH]n1] | 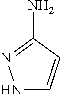 | LCMS m/z 366.82 [M + H]⁺. |
| 71 | As for Compound 1 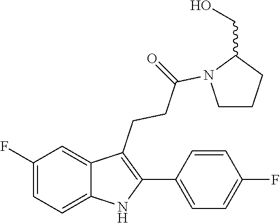 [SMILES: O=C(CCc1c(-c2ccc(F)cc2)[nH]c2ccc(F)cc12)N1CCCC1CO] | 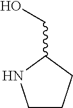 | LCMS m/z 385 [M + H]⁺. |
| 72 | As for Compound 1 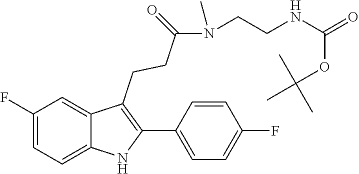 [SMILES: CN(CCNC(=O)OC(C)(C)C)C(=O)CCc1c(-c2ccc(F)cc2)[nH]c2ccc(F)cc12] | 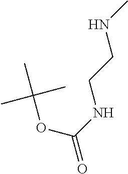 | LCMS m/z 456.9 [M + H]⁺. |
| 73 | As for Compound 1 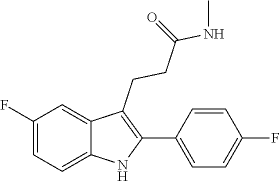 [SMILES: CNC(=O)CCc1c(-c2ccc(F)cc2)[nH]c2ccc(F)cc12] | 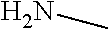 | LCMS m/z 314.92 [M + H]⁺. |
| 74 | As for Compound 1 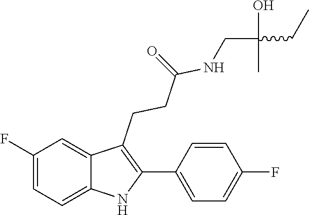 [SMILES: CCC(C)(O)CNC(=O)CCc1c(-c2ccc(F)cc2)[nH]c2ccc(F)cc12] | 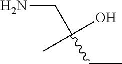 | LCMS m/z 387.35 [M + H]⁺. |
| 75 | As for Compound 1 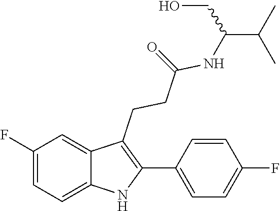 [SMILES: CC(C)C(CO)NC(=O)CCc1c(-c2ccc(F)cc2)[nH]c2ccc(F)cc12] | 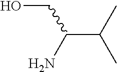 | ¹H NMR (300 MHz, Methanol-d₄) δ 7.77-7.57 (m, 2H), 7.41-7.09 (m, 4H), 6.87 (ddd, J = 9.4, 8.7, 2.5 Hz, 1H), 3.65 (h, J = 5.7, 5.3 Hz, 1H), 3.55-3.40 (m, 2H), 3.15 (td, J = 7.4, 1.9 Hz, 2H), 2.59 (ddd, J = 9.4, 6.4, 1.3 Hz, 2H), 1.78 (h, J = 6.8 Hz, 1H), 0.82 (d, J = 6.8 Hz, 3H), 0.75 (d, J = 6.8 Hz, 3H); LCMS m/z 387.11 [M + H]⁺. |
| 76 | As for Compound 1 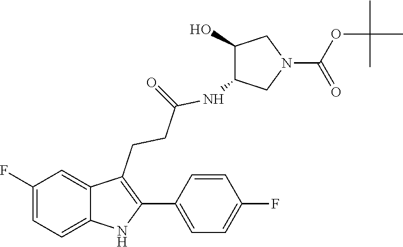 [SMILES: CC(C)(C)OC(=O)N1C[C@H](NC(=O)CCc2c(-c3ccc(F)cc3)[nH]c3ccc(F)cc23)[C@@H](O)C1] | 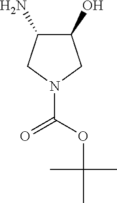 | ¹H NMR (400 MHz, Methanol-d₄) δ 7.64 (ddd, J = 8.8, 5.2, 2.2 Hz, 2H), 7.30 (td, J = 6.2, 5.7, 2.9 Hz, 2H), 7.25- 7.18 (m, 2H), 6.87 (tt, J = 8.8, 2.2 Hz, 1H), 4.02 (ddt, J = 11.1, 5.5, 2.3 Hz, 1H), 3.89 (ddt, J = 9.0, 4.4, 2.4 Hz, 1H), 3.55 (ddd, J = 11.5, 5.9, 4.2 Hz, 1H), 3.25-3.08 (m, 4H), 2.60-2.43 (m, 2H), 1.43 (d, J = 1.0 Hz, 9H); LCMS m/z 486.17 [M + H]⁺. |
| 77 | As for Compound 1 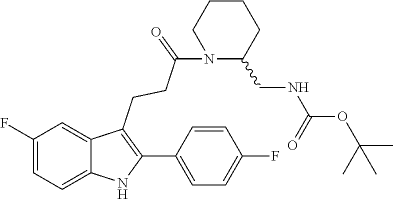 [SMILES: CC(C)(C)OC(=O)NCC1CCCCN1C(=O)CCc1c(-c2ccc(F)cc2)[nH]c2ccc(F)cc12] | | LCMS m/z 496.17 [M + H]⁺. |
| 78 | As for Compound 1 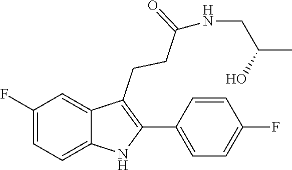 [SMILES: C[C@H](O)CNC(=O)CCc1c(-c2ccc(F)cc2)[nH]c2ccc(F)cc12] | 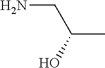 | ¹H NMR (400 MHz, Methanol-d₄) δ 7.66-7.58 (m, 2H), 7.40-7.26 (m, 2H), 7.27-7.16 (m, 2H), 6.87 (ddd, J = 9.5, 8.8, 2.5 Hz, 1H), 3.71 (pd, J = 6.3, 5.3 Hz, 1H), 3.22-2.94 (m, 4H), 2.59- 2.47 (m, 2H), 1.02 (d, J = 6.3 Hz, 3H); LCMS m/z 359.17 [M + H]⁺. |
| 79 | As for Compound 1 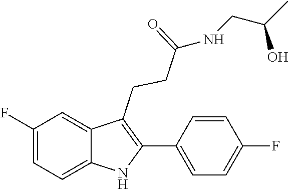 [SMILES: C[C@@H](O)CNC(=O)CCc1c(-c2ccc(F)cc2)[nH]c2ccc(F)cc12] | 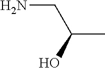 | ¹H NMR (300 MHz, Methanol-d₄) δ 7.73-7.56 (m, 2H), 7.37-7.15 (m, 4H), 6.97-6.78 (m, 1H), 3.71 (td, J = 6.4, 5.4 Hz, 1H), 3.19- 3.02 (m, 4H), 2.54 (dd, J = 8.9, 6.8 Hz, 2H), 1.02 (d, J = 6.3 Hz, 3H); LCMS m/z 359.1 [M + H]⁺. |
| 80 | As for Compound 1 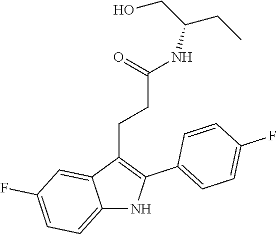 [SMILES: CC[C@@H](CO)NC(=O)CCc1c(-c2ccc(F)cc2)[nH]c2ccc(F)cc12] | 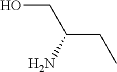 | ¹H NMR (300 MHz, Chloroform-d) δ 8.06 (s, 1H), 7.63-7.50 (m, 2H), 7.35- 7.28 (m, 2H), 7.27-7.12 (m, 2H), 6.98 (td, J = 9.0, 2.5 Hz, 1H), 5.39 (d, J = 7.7 Hz, 1H), 3.81-3.68 (m, 1H), 3.55 (dd, J = 11.1, 3.4 Hz, 1H), 3.45 (dd, J = 11.1, 5.7 Hz, 1H), 3.22 (dd, J = 8.2, 6.9 Hz, 2H), 2.58 (t, J = 7.5 Hz, 2H), 1.59- 1.39 (m, 1H), 1.32 (dq, J = 14.2, 7.4 Hz, 1H), 0.79 (t, J = 7.5 Hz, 3H); LCMS m/z 373.11 [M + H]⁺. |
| 81 | As for Compound 1 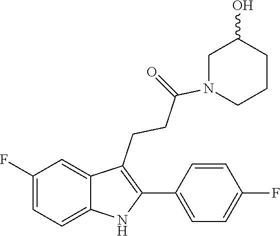 [SMILES: O=C(CCc1c(-c2ccc(F)cc2)[nH]c2ccc(F)cc12)N1CCCC(O)C1] | 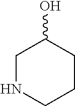 | LCMS m/z 385 [M + H]⁺. |
| 82 | As for Compound 1 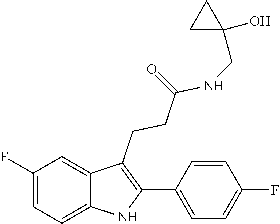 [SMILES: O=C(CCc1c(-c2ccc(F)cc2)[nH]c2ccc(F)cc12)NCC1(O)CC1] | 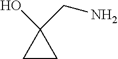 | LCMS m/z 371.3 [M + H]⁺. |
| 83 | As for Compound 1 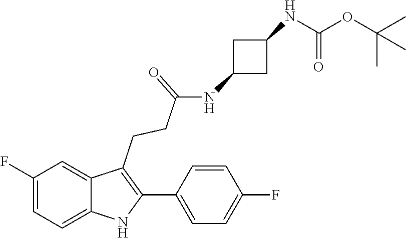 [SMILES: CC(C)(C)OC(=O)N[C@H]1C[C@@H](NC(=O)CCc2c(-c3ccc(F)cc3)[nH]c3ccc(F)cc23)C1] | 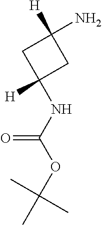 | LCMS m/z 467.85 [M + H]⁺. |
| 84 | As for Compound 1 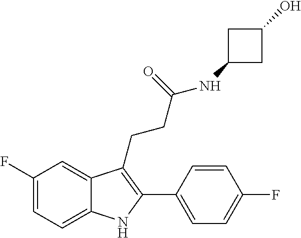 [SMILES: O=C(CCc1c(-c2ccc(F)cc2)[nH]c2ccc(F)cc12)N[C@H]1C[C@H](O)C1] | 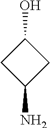 | LCMS m/z 371.23 [M + H]⁺. |
| 85 | As for Compound 1 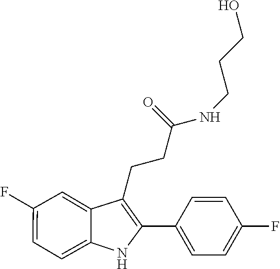 [SMILES: O=C(CCc1c(-c2ccc(F)cc2)[nH]c2ccc(F)cc12)NCCCO] | 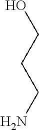 | LCMS m/z 359.28 [M + H]⁺. |
| 86 | As for Compound 1 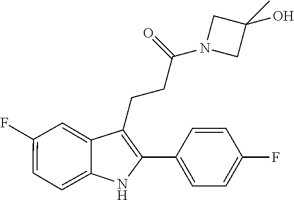 [SMILES: CC1(O)CN(C(=O)CCc2c(-c3ccc(F)cc3)[nH]c3ccc(F)cc23)C1] | 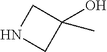 | LCMS m/z 371.27 [M + H]⁺. |
| 87 | As for Compound 1 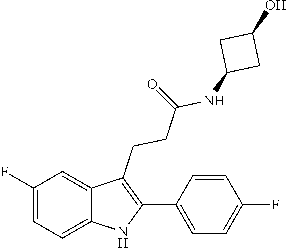 [SMILES: O=C(CCc1c(-c2ccc(F)cc2)[nH]c2ccc(F)cc12)N[C@H]1C[C@@H](O)C1] | 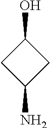 | LCMS m/z 371.27 [M + H]⁺. |
| 88 | As for Compound 1 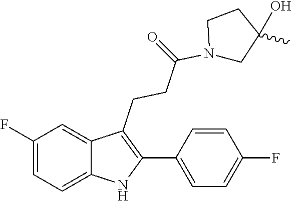 [SMILES: CC1(O)CCN(C(=O)CCc2c(-c3ccc(F)cc3)[nH]c3ccc(F)cc23)C1] | 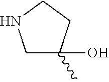 | LCMS m/z 385.3 [M + H]⁺. |
| 89 | As for Compound 1 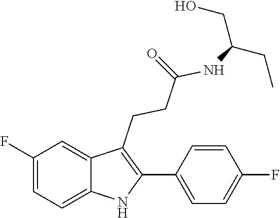 [SMILES: CC[C@H](CO)NC(=O)CCc1c(-c2ccc(F)cc2)[nH]c2ccc(F)cc12] | 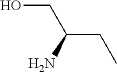 | ¹H NMR (300 MHz, Chloroform-d) δ 8.06 (d, J = 23.2 Hz, 1H), 7.63-7.49 (m, 2H), 7.37-7.25 (m, 3H), 7.26- 7.12 (m, 2H), 6.98 (td, J = 9.0, 2.5 Hz, 1H), 5.42 (d, J = 7.8 Hz, 1H), 3.86-3.66 (m, 0H), 3.55 (dd, J = 11.1, 3.4 Hz, 1H), 3.44 (dd, J = 11.1, 5.7 Hz, 1H), 3.21 (dd, J = 8.2, 6.9 Hz, 2H), 2.58 (t, J = 7.5 Hz, 2H), 1.59-1.14 (m, 2H), 0.79 (t, J = 7.4 Hz, 3H); LCMS m/z 373.11 [M + H]⁺. |
| 90 | As for Compound 1 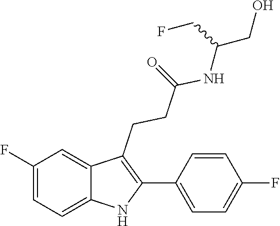 [SMILES: O=C(CCc1c(-c2ccc(F)cc2)[nH]c2ccc(F)cc12)NC(CO)CF] | 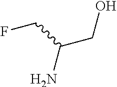 | ¹H NMR (300 MHz, Methanol-d₄) δ 7.75-7.58 (m, 2H), 7.40-7.11 (m, 4H), 6.96-6.75 (m, 1H), 4.61- 4.18 (m, 2H), 4.15-4.01 (m, 1H), 3.57-3.44 (m, 2H), 3.26- 3.06 (m, 2H), 2.61-2.46 (m, 2H); LCMS m/z 377.15 [M + H]⁺. |

¹C8 BEH Column was used for purification.

### Compound 91

### 3-[5,7-difluoro-2-(4-fluorophenyl)-1H-indol-3-yl]-N-[(3S,4S)-4-hydroxy-2-oxo-pyrrolidin-3-yl]propanamide (91)

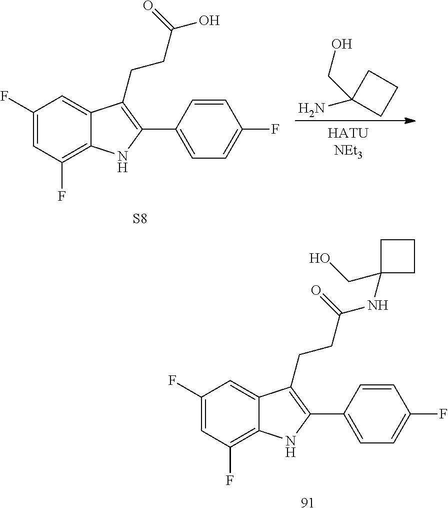 [SMILES: O=C(O)CCc1c(-c2ccc(F)cc2)[nH]c2c(F)cc(F)cc12 ; NC1(CO)CCC1 ; "S8" ; O=C(CCc1c(-c2ccc(F)cc2)[nH]c2c(F)cc(F)cc12)NC1(CO)CCC1 ; "91"]

### Preparation of 3-[5,7-difluoro-2-(4-fluorophenyl)-1H-indol-3-yl]-N-[(3S,4S)-4-hydroxy-2-oxo-pyrrolidin-3-yl]propanamide (91)

To a solution (1-aminocyclobutyl)methanol (13 mg, 0.1285 mmol) in DMSO (1 mL) was added 3-[5,7-difluoro-2-(4-fluorophenyl)-1H-indol-3-yl]propanoic acid S8 (27 mg, 0.08457 mmol), HATU (45 mg, 0.1183 mmol) and NEt₃ (40 μL, 0.2870 mmol). The mixture was allowed to stir at room temperature for 5 hours. Purification by reversed-phase HPLC (Method: C18 Waters Sunfire column (30×150 mm, 5 micron). Gradient: MeCN in H₂O with 0.1% trifluoroacetic acid) afforded the product 3-[5,7-difluoro-2-(4-fluorophenyl)-1H-indol-3-yl]-N-[1-(hydroxymethyl)-cyclobutyl]propanamide (91) (20 mg, 59%). ¹H NMR (300 MHz, Methanol-d₄) δ 7.76-7.55 (m, 2H), 7.32-7.04 (m, 3H), 6.72 (ddd, J=11.1, 9.6, 2.2 Hz, 1H), 3.66 (s, 2H), 3.21-3.01 (m, 2H), 2.56-2.38 (m, 2H), 2.18-1.92 (m, 4H), 1.84-1.65 (m, 2H). LCMS m/z 403.17 [M+H]⁺.

### Compound 92

### 3-[5,7-difluoro-2-(4-fluorophenyl)-1H-indol-3-yl]-N-(5-methyl-1,2,4-oxadiazol-3-yl)propanamide (92)

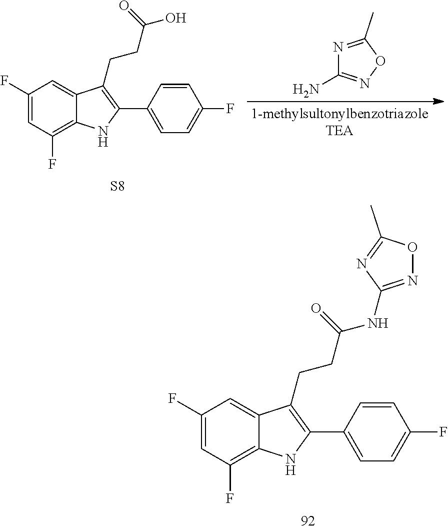

### Preparation of 3-[5,7-difluoro-2-(4-fluorophenyl)-1H-indol-3-yl]-N-(5-methyl-1,2,4-oxadiazol-3-yl)propanamide (92)

A microwave tube was charged with a solution of 3-[5,7-difluoro-2-(4-fluorophenyl)-1H-indol-3-yl]propanoic acid S8 (18 mg, 0.056 mmol), 5-methyl-1,2,4-oxadiazol-3-amine (11.2 mg, 0.1130 mmol), and 1-methylsulfonylbenzotriazole (23 mg, 0.1122 mmol) in THF (1 mL). TEA (35 μL, 0.2511 mmol) was added and the reaction was heated at 130° C. for 30 minutes. Purification by reversed-phase HPLC (Method: C18 Waters Sunfire column (30×150 mm, 5 micron). Gradient: MeCN in H₂O with 0.2% formic acid) afforded 3-[5,7-difluoro-2-(4-fluorophenyl)-1H-indol-3-yl]-N-(5-methyl-1,2,4-oxadiazol-3-yl)propanamide (92) (2.1 mg, 9%). ¹H NMR (300 MHz, Methanol-d₄) δ 7.74-7.57 (m, 2H), 7.34-7.20 (m, 2H), 7.11 (dd, J=9.4, 2.2 Hz, 1H), 6.73 (ddd, J=11.1, 9.6, 2.2 Hz, 1H), 3.30 (s, 3H), 3.20-3.01 (m, 2H), 2.68-2.45 (m, 2H). LCMS m/z 401.12 [M+H]⁺.

### Compound 93

### 3-[5,7-difluoro-2-(4-fluorophenyl)-1H-indol-3-yl]-N-(5-oxo-1,2-dihydropyrrol-4-yl)propanamide (93)

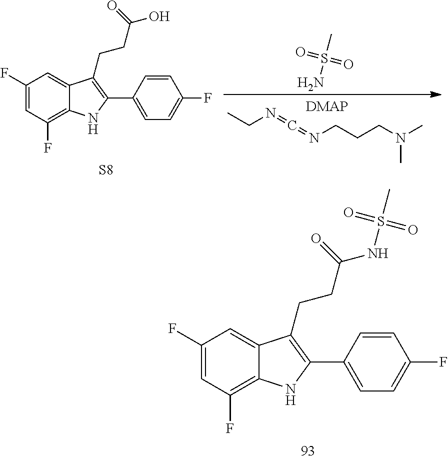

### Preparation of 3-[5,7-difluoro-2-(4-fluorophenyl)-1H-indol-3-yl]-N-(5-oxo-1,2-dihydropyrrol-4-yl)propanamide (93)

To 3-[5,7-difluoro-2-(4-fluorophenyl)-1H-indol-3-yl]propanoic acid S8 (29 mg, 0.089 mmol), methanesulfonamide (17 mg, 0.1787 mmol) and DMAP (22 mg, 0.1801 mmol) in DMF (1 mL) was added 3-(ethyliminomethyleneamino)-N,N-dimethyl-propan-1-amine (Hydrochloride salt) (26 mg, 0.1356 mmol). Reaction was stirred for 1 hour. After 1 hour a further eq. of DMAP was added. Reaction was stirred at room temperature for 48 hours. Purification by reversed-phase HPLC (Method: C18 Waters Sunfire column (30×150 mm, 5 micron). Gradient: MeCN in H₂O with 0.1% trifluoroacetic acid) afforded the product 3-[5,7-difluoro-2-(4-fluorophenyl)-1H-indol-3-yl]-N-methylsulfonyl-propanamide (93) (18 mg, 50%). ¹H NMR (300 MHz, Methanol-d₄) δ 7.79-7.54 (m, 2H), 7.34-7.19 (m, 2H), 7.17 (dd, J=9.5, 2.2 Hz, 1H), 6.76 (ddd, J=11.1, 9.6, 2.2 Hz, 1H), 3.24-3.02 (m, 2H), 3.15 (s, 3H), 2.73-2.50 (m, 2H). LCMS m/z 396.88 [M+H]⁺.

### Compound 94

### 3-[5,7-difluoro-2-(4-fluorophenyl)-1H-indol-3-yl]-N-(5-oxo-1,2-dihydropyrrol-4-yl)propanamide (94)

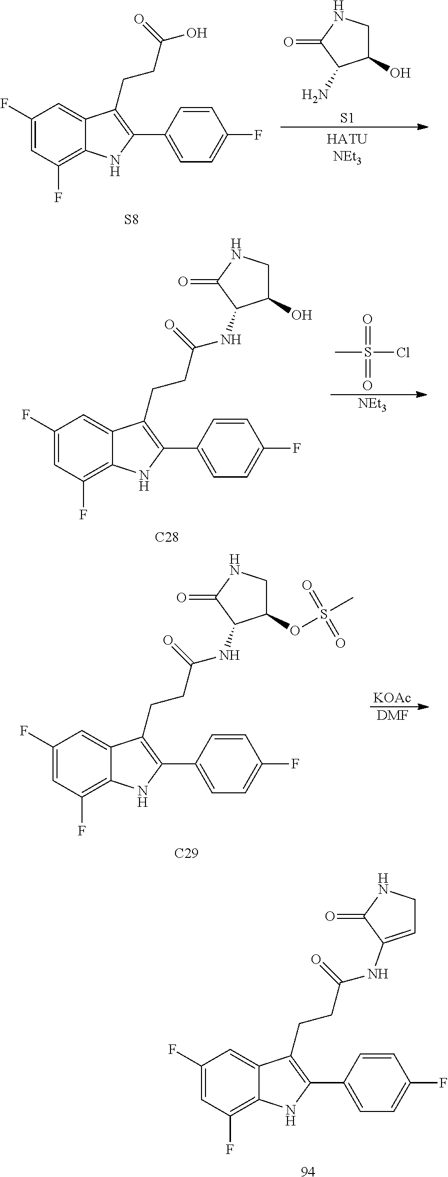

### Step 1. Synthesis of 3-[5,7-difluoro-2-(4-fluorophenyl)-1H-indol-3-yl]-N-[(3S,4R)-4-hydroxy-2-oxo-pyrrolidin-3-yl]propanamide (C28)

To a solution of 3-[5,7-difluoro-2-(4-fluorophenyl)-1H-indol-3-yl]-N-[(3S,4S)-4-hydroxy-2-oxo-pyrrolidin-3-yl]propanamide S1 in DMSO (1 mL) was added 3-[5,7-difluoro-2-(4-fluorophenyl)-1H-indol-3-yl]propanoic acid S8 (25 mg, 0.08 mmol), HATU (33 mg, 0.09 mmol) and NEt₃ (30 μL, 0.22 mmol). The mixture was allowed to stir at room temperature for 2 hours. The mixture was then purified by reversed-phase HPLC (Method: C18 Waters Sunfire column (30×150 mm, 5 micron). Gradient: MeCN in H₂O with 0.2% formic acid) to afford the product C28 (6 mg, 40%). ¹H NMR (300 MHz, Methanol-d₄) δ 7.68 (ddd, J=9.2, 5.1, 2.3 Hz, 2H), 7.37-7.19 (m, 3H), 6.75 (ddt, J=11.4, 9.6, 1.9 Hz, 1H), 4.68 (d, J=5.1 Hz, 1H), 4.40 (dd, J=5.1, 3.9 Hz, 1H), 3.65-3.57 (m, 1H), 3.26 (d, J=11.3 Hz, 1H), 3.20-3.08 (m, 2H), 2.75-2.64 (m, 2H). LCMS m/z 418.1 [M+H]⁺.

### Step 2. Synthesis of [(3R,4S)-4-[3-[5,7-difluoro-2-(4-fluorophenyl)-1H-indol-3-yl]propanoyl-amino]-5-oxo-pyrrolidin-3-yl] methanesulfonate (C29)

To a solution of 3-[5,7-difluoro-2-(4-fluorophenyl)-1H-indol-3-yl]-N-[(3S,4R)-4-hydroxy-2-oxo-pyrrolidin-3-yl]propanamide C28 (105 mg, 0.2516 mmol) in DCM (2 mL) was added Et₃N (53 μL, 0.3803 mmol). After cooling to 0° C. MSCl (24 μL, 0.3101 mmol) was added and, after stirring for 5 minutes at 0° C., the mixture was allowed to warm up to room temperature and stirred for a further 2.5 hours. The mixture was evaporated in vacuo. Purification by silica gel chromatography (Eluent: 50% EtOAc in heptane) afforded the product [(3R,4S)-4-[3-[5,7-difluoro-2-(4-fluorophenyl)-1H-indol-3-yl]propanoylamino]-5-oxo-pyrrolidin-3-yl]methanesulfonate (C29) (45 mg, 31%). ¹H NMR (300 MHz, Methanol-d₄) δ 7.61 (ddt, J=8.3, 5.2, 2.6 Hz, 2H), 7.32-7.16 (m, 2H), 7.12 (dd, J=9.4, 2.2 Hz, 1H), 6.72 (ddd, J=11.1, 9.6, 2.2 Hz, 1H), 5.46 (dd, J=5.6, 4.1 Hz, 1H), 4.45 (d, J=5.6 Hz, 1H), 3.61 (dd, J=12.0, 4.2 Hz, 1H), 3.23-3.08 (m, 3H), 3.04 (s, 3H), 2.84-2.59 (m, 2H). LCMS m/z 496.35 [M+H]⁺.

### Step 3. Synthesis of 3-[5,7-difluoro-2-(4-fluorophenyl)-1H-indol-3-yl]-N-(5-oxo-1,2-dihydro-pyrrol-4-yl)propanamide (94)

A mixture of [(3R,4S)-4-[3-[5,7-difluoro-2-(4-fluorophenyl)-1H-indol-3-yl]propanoylamino]-5-oxo-pyrrolidin-3-yl] methanesulfonate C29 (15 mg, 0.03027 mmol) and KOAc (9 mg, 0.09170 mmol) in DMF (2 mL) was heated to 80° C. overnight. Reaction was cooled to room temperature, diluted with water followed by extraction with EtOAc (3×5 mL). Combined organic fractions were washed with H₂O (2 mL), and brine (2 mL). The organic layer was dried over sodium sulfate and filtered. Purification by silica gel chromatography (Eluent: EtOAc) afforded the product 3-[5,7-difluoro-2-(4-fluorophenyl)-1H-indol-3-yl]-N-(5-oxo-1,2-dihydropyrrol-4-yl)propanamide (94) (5.4 mg, 40%). ¹H NMR (300 MHz, Methanol-d₄) δ 7.72-7.54 (m, 2H), 7.29-7.04 (m, 4H), 6.71 (ddd, J=11.1, 9.6, 2.2 Hz, 1H), 3.95 (d, J=2.2 Hz, 2H), 3.18 (dd, J=8.5, 6.8 Hz, 2H), 2.71 (dd, J=8.5, 6.8 Hz, 2H). LCMS m/z 400.14 [M+H]⁺.

### Compounds 95-193

Compounds 95-193 (see Table 3) were prepared in a single step from intermediate S8 using the appropriate reagent and using the amide formation methods as described for compounds 91-94 (using coupling reagents such as HATU, or 1-methylsulfonylbenzotriazole). Amines were prepared by methods described above or obtained from commercial sources. Any modifications to methods are noted in Table 3 and accompanying footnotes.

TABLE 3
Method of preparation, structure and physicochemical data for compounds 95-193

| | | Amine | ¹H NMR; LCMS m/z |
|---|---|---|---|
| Compound | Method/Product | Reagent | [M + H]⁺ |
| 95 | As for Compound 91^{1,2,4,5,6,} 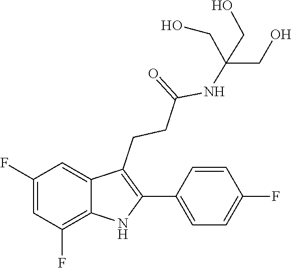 [SMILES: O=C(CCc1c(-c2ccc(F)cc2)[nH]c2c(F)cc(F)cc12)NC(CO)(CO)CO] | 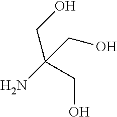 | ¹H NMR (400 MHz, Methanol-d₄) δ 7.70-7.60 (m, 2H), 7.28-7.21 (m, 2H), 7.19 (dd, J = 9.5, 2.2 Hz, 1H), 6.73 (ddd, J = 11.0, 9.6, 2.2 Hz, 1H), 3.62 (s, 6H), 3.17-3.05 (m, 2H), 2.62-2.51 (m, 2H); LCMS m/z 423.45 [M + H]⁺ |
| 96 | As for Compound 91⁷ 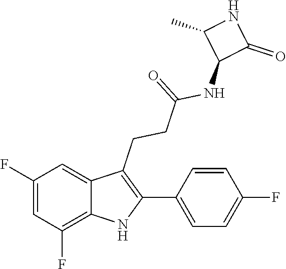 [SMILES: C[C@@H]1NC(=O)[C@H]1NC(=O)CCc1c(-c2ccc(F)cc2)[nH]c2c(F)cc(F)cc12] | 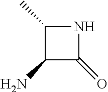 | ¹H NMR (300 MHz, Methanol-d₄) δ 8.60 (d, J = 8.0 Hz, 1H), 7.73-7.59 (m, 2H), 7.32-7.18 (m, 2H), 7.15 (dd, J = 9.4, 2.2 Hz, 1H), 6.72 (ddd, J = 11.1, 9.6, 2.2 Hz 1H) 4.40-4.25 (m, 1H), 3.53-3.39 (m, 1H), 3.22-3.04 (m, 3H), 2.60-2.44 (m, 2H), 1.30 (d, J = 6.2 Hz, 3H); LCMS m/z 402.3 [M + H]⁺ |
| 97 | As for Compound 91 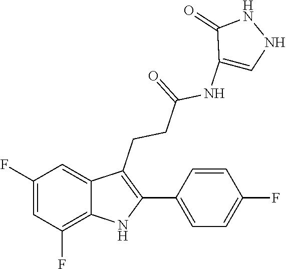 [SMILES: O=C(CCc1c(-c2ccc(F)cc2)[nH]c2c(F)cc(F)cc12)Nc1c[nH][nH]c1=O] | 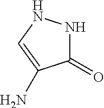 | LCMS m/z 401.15 [M + H]⁺ |
| 98 | As for Compound 91³ 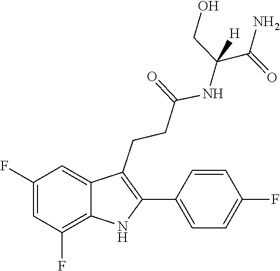 [SMILES: NC(=O)[C@@H](CO)NC(=O)CCc1c(-c2ccc(F)cc2)[nH]c2c(F)cc(F)cc12] | 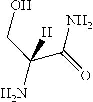 | LCMS m/z 406.13 [M + H]⁺ |
| 99 | As for Compound 91³ 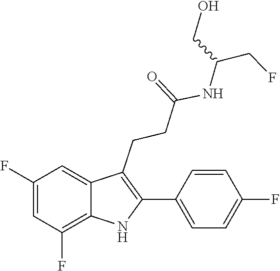 [SMILES: O=C(CCc1c(-c2ccc(F)cc2)[nH]c2c(F)cc(F)cc12)NC(CO)CF] | 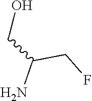 | ¹H NMR (300 MHz, Methanol-d₄) δ 7.78-7.59 (m, 2H), 7.35-7.11 (m, 3H), 6.75 (ddd, J = 11.1, 9.6, 2.2 Hz, 1H), 4.62-4.17 (m, 2H), 4.05 (dq, J = 22.0, 5.3 Hz, 1H), 3.59-3.51 (m, 2H), 3.23-3.08 (m, 2H), 2.62-2.49 (m, 2H); LCMS m/z 395.27 [M + H]⁺ |
| 100 | As for Compound 91³ 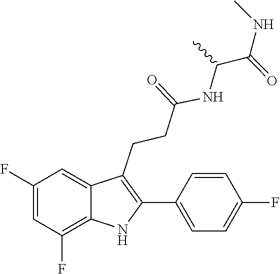 [SMILES: CNC(=O)C(C)NC(=O)CCc1c(-c2ccc(F)cc2)[nH]c2c(F)cc(F)cc12] | 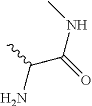 | LCMS m/z 404.15 [M + H]⁺ |
| 101 | As for Compound 91 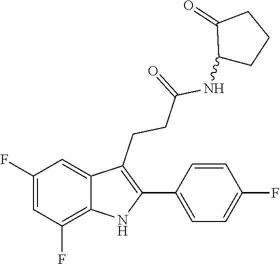 [SMILES: O=C(CCc1c(-c2ccc(F)cc2)[nH]c2c(F)cc(F)cc12)NC1CCCC1=O] | 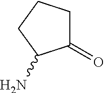 | LCMS m/z 400.8 [M + H]⁺ |
| 102 | As for Compound 91³ 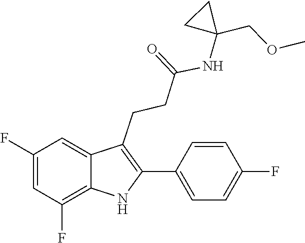 [SMILES: COCC1(NC(=O)CCc2c(-c3ccc(F)cc3)[nH]c3c(F)cc(F)cc23)CC1] | 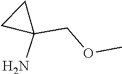 | LCMS m/z 403.17 [M + H]⁺ |
| 103 | As for Compound 91³ 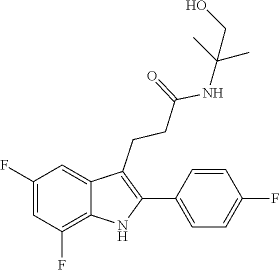 [SMILES: CC(C)(CO)NC(=O)CCc1c(-c2ccc(F)cc2)[nH]c2c(F)cc(F)cc12] | 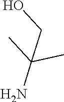 | LCMS m/z 391.19 [M + H]⁺ |
| 104 | As for Compound 91 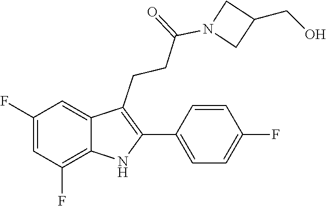 [SMILES: O=C(CCc1c(-c2ccc(F)cc2)[nH]c2c(F)cc(F)cc12)N1CC(CO)C1] | 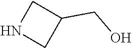 | LCMS m/z 389.13 [M + H]⁺ |
| 105 | As for Compound 91 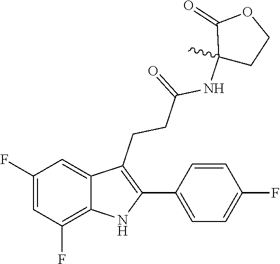 [SMILES: CC1(NC(=O)CCc2c(-c3ccc(F)cc3)[nH]c3c(F)cc(F)cc23)CCOC1=O] | 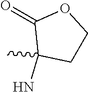 | LCMS m/z 417.01 [M + H]⁺ |
| 106 | As for Compound 91³ 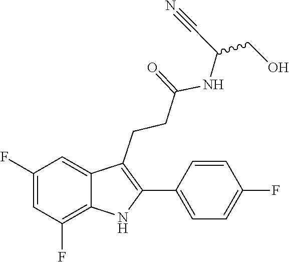 [SMILES: N#CC(CO)NC(=O)CCc1c(-c2ccc(F)cc2)[nH]c2c(F)cc(F)cc12] | 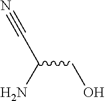 | LCMS m/z 387.99 [M + H]⁺ |
| 107 | As for Compound 91³ 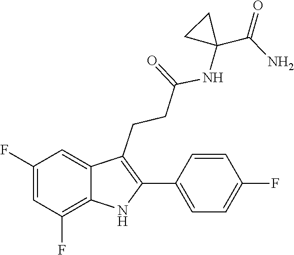 [SMILES: NC(=O)C1(NC(=O)CCc2c(-c3ccc(F)cc3)[nH]c3c(F)cc(F)cc23)CC1] | 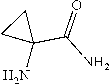 | LCMS m/z 402.13 [M + H]⁺ |
| 108 | As for Compound 92 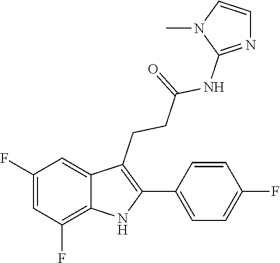 [SMILES: Cn1ccnc1NC(=O)CCc1c(-c2ccc(F)cc2)[nH]c2c(F)cc(F)cc12] | 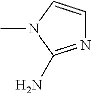 | LCMS m/z 399.17 [M + H]⁺ |
| 109 | As for Compound 91³ 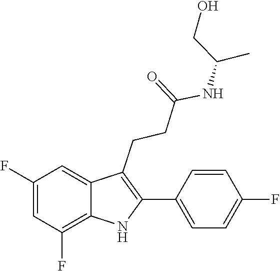 [SMILES: C[C@@H](CO)NC(=O)CCc1c(-c2ccc(F)cc2)[nH]c2c(F)cc(F)cc12] | 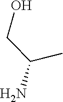 | ¹H NMR (300 MHz, Chloroform-d) δ 8.28 (s, 1H), 7.63-7.50 (m, 2H), 7.25-7.14 (m, 2H), 7.10 (dd, J = 9.1, 2.2 Hz, 1H), 6.86-6.68 (m, 1H), 5.54 (d, J = 7.3 Hz, 1H), 3.99 (dtt, J = 10.6, 7.2, 3.7 Hz, 1H), 3.57 (dd, J = 11.1, 3.5 Hz, 1H), 3.43 (dd, J = 11.0, 6.0 Hz, 1H), 3.18 (dd, J = 8.5, 6.9 Hz, 2H), 2.53 (dd, J = 8.4, 6.9 Hz, 2H), 1.04 (d, J = 6.9 Hz, 3H); LCMS m/z 377.2 [M + H]⁺ |
| 110 | As for Compound 91³ 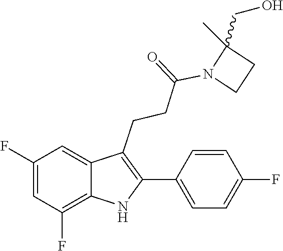 [SMILES: CC1(CO)CCN1C(=O)CCc1c(-c2ccc(F)cc2)[nH]c2c(F)cc(F)cc12] | 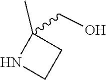 | LCMS m/z 403.14 [M + H]⁺ |
| 111 | As for Compound 91³ 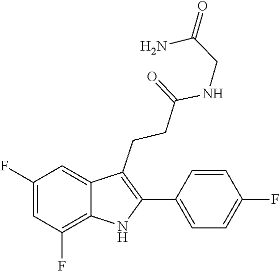 [SMILES: NC(=O)CNC(=O)CCc1c(-c2ccc(F)cc2)[nH]c2c(F)cc(F)cc12] | 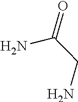 | ¹H NMR (300 MHz, Methanol-d₄) δ 7.71-7.55 (m, 2H), 7.34-7.08 (m, 3H), 6.72 (ddd, J = 11.4, 9.7, 2.2 Hz, 1H), 3.77 (s, 2H), 3.22-3.02 (m, 2H), 2.68-2.39 (m, 2H); LCMS m/z 376.1 [M + H]⁺ |
| 112 | As for Compound 91 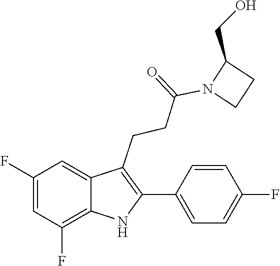 [SMILES: O=C(CCc1c(-c2ccc(F)cc2)[nH]c2c(F)cc(F)cc12)N1CC[C@@H]1CO] | 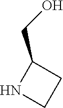 | LCMS m/z 389.13 [M + H]⁺ |
| 113 | As for Compound 91 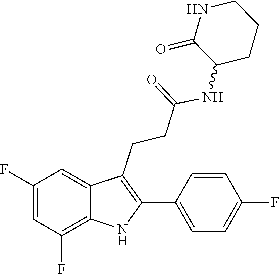 [SMILES: O=C(CCc1c(-c2ccc(F)cc2)[nH]c2c(F)cc(F)cc12)NC1CCCNC1=O] | 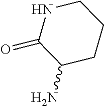 | LCMS m/z 415.78 [M + H]⁺ |
| 114 | As for Compound 91 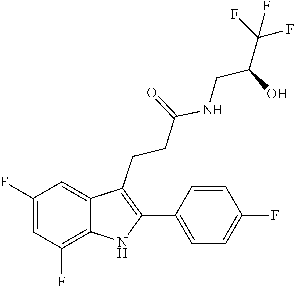 [SMILES: O=C(CCc1c(-c2ccc(F)cc2)[nH]c2c(F)cc(F)cc12)NC[C@H](O)C(F)(F)F] | 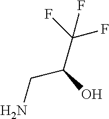 | LCMS m/z 431.09 [M + H]⁺ |
| 115 | As for Compound 91³ 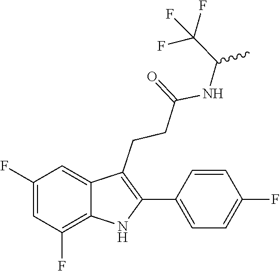 [SMILES: CC(NC(=O)CCc1c(-c2ccc(F)cc2)[nH]c2c(F)cc(F)cc12)C(F)(F)F] | 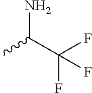 | LCMS m/z 415.13 [M + H]⁺ |
| 116 | As for Compound 91³ 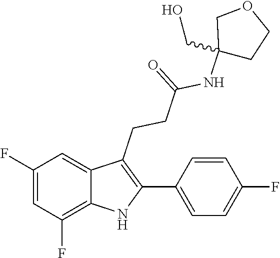 [SMILES: O=C(CCc1c(-c2ccc(F)cc2)[nH]c2c(F)cc(F)cc12)NC1(CO)CCOC1] | 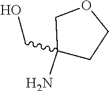 | LCMS m/z 419.17 [M + H]⁺ |
| 117 | As for Compound 91 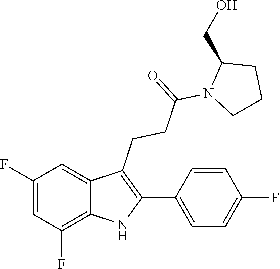 [SMILES: O=C(CCc1c(-c2ccc(F)cc2)[nH]c2c(F)cc(F)cc12)N1CCC[C@@H]1CO] | 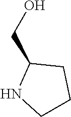 | LCMS m/z 403.17 [M + H]⁺ |
| 118 | As for Compound 91³ 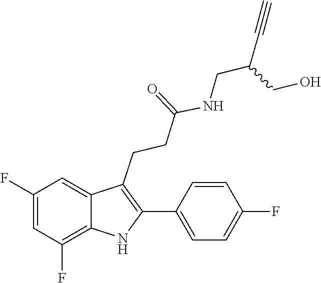 | 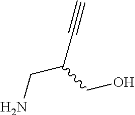 | LCMS m/z 387.15 [M + H]⁺ |
| 119 | As for Compound 91³ 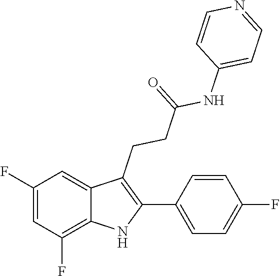 [SMILES: O=C(CCc1c(-c2ccc(F)cc2)[nH]c2c(F)cc(F)cc12)Nc1ccncc1] | 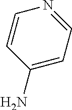 | ¹H NMR (300 MHz, Methanol-d₄) δ 8.46-8.30 (m, 2H), 7.78-7.52 (m, 4H), 7.30-7.07 (m, 3H), 6.74 (ddd, J = 11.1, 9.6, 2.2 Hz, 1H), 3.25 (dd, J = 8.4, 6.8 Hz, 2H), 2.73 (dd, J = 8.4, 6.8 Hz, 2H); LCMS m/z 396.14 [M + H]⁺ |
| 120 | As for Compound 91³ 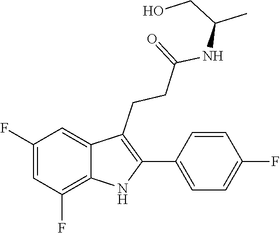 [SMILES: C[C@H](CO)NC(=O)CCc1c(-c2ccc(F)cc2)[nH]c2c(F)cc(F)cc12] | 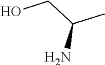 | ¹H NMR (300 MHz, Chloroform-d) δ 8.14 (s, 1H), 7.63-7.43 (m, 2H), 7.27-7.17 (m, 2H), 7.11 (dd, J = 9.1, 2.2 Hz, 1H), 6.85-6.70 (m, 1H), 5.43 (s, 1H), 4.00 (ddt, J = 10.2, 6.8, 3.5 Hz, 1H), 3.58 (dd, J = 11.0, 3.5 Hz, 1H), 3.44 (dd, J = 11.0, 5.9 Hz, 1H), 3.27- 3.09 (m, 2H), 2.59-2.41 (m, 2H), 1.05 (d, J = 6.9 Hz, 3H); LCMS m/z 377.2 [M + H]⁺ |
| 121 | As for Compound 91³ 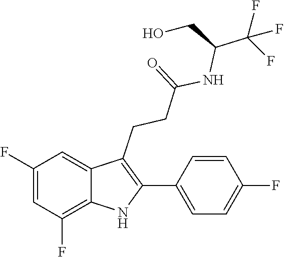 [SMILES: O=C(CCc1c(-c2ccc(F)cc2)[nH]c2c(F)cc(F)cc12)N[C@@H](CO)C(F)(F)F] | 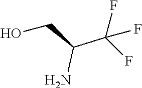 | ¹H NMR (300 MHz, Methanol-d₄) δ 7.71-7.54 (m, 2H), 7.31-7.13 (m, 3H), 6.72 (ddd, J = 11.1, 9.6, 2.2 Hz, 1H), 4.64-4.54 (m, 1H), 3.80-3.69 (m, 1H), 3.64 (dd, J = 11.8, 6.6 Hz, 1H), 3.20-3.09 (m, 2H), 2.71-2.50 (m, 2H); LCMS m/z 431.09 [M + H]⁺ |
| 122 | As for Compound 91³ 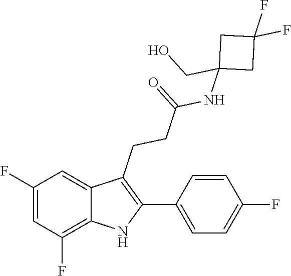 [SMILES: O=C(CCc1c(-c2ccc(F)cc2)[nH]c2c(F)cc(F)cc12)NC1(CO)CC(F)(F)C1] | 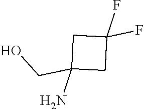 | LCMS m/z 439.23 [M + H]⁺ |
| 123 | As for Compound 91 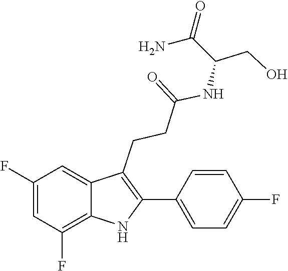 [SMILES: NC(=O)[C@H](CO)NC(=O)CCc1c(-c2ccc(F)cc2)[nH]c2c(F)cc(F)cc12] | 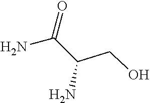 | LCMS m/z 406.13 [M + H]⁺ |
| 124 | As for Compound 91³ 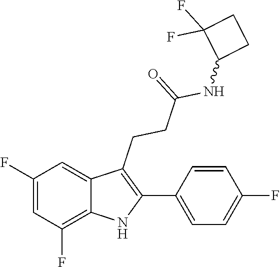 [SMILES: O=C(CCc1c(-c2ccc(F)cc2)[nH]c2c(F)cc(F)cc12)NC1CCC1(F)F] | 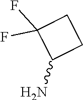 | LCMS m/z 409.14 [M + H]⁺ |
| 125 | As for Compound 92 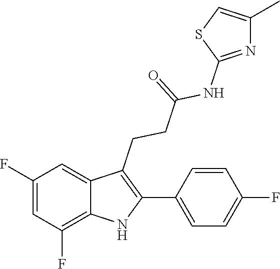 [SMILES: Cc1csc(NC(=O)CCc2c(-c3ccc(F)cc3)[nH]c3c(F)cc(F)cc23)n1] | 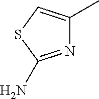 | LCMS m/z 416.11 [M + H]⁺ |
| 126 | As for Compound 91 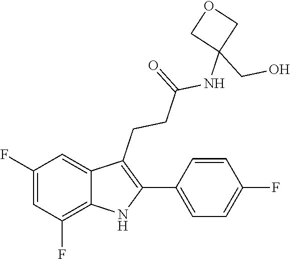 [SMILES: O=C(CCc1c(-c2ccc(F)cc2)[nH]c2c(F)cc(F)cc12)NC1(CO)COC1] | 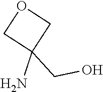 | LCMS m/z 405.16 [M + H]⁺ |
| 127 | As for Compound 91 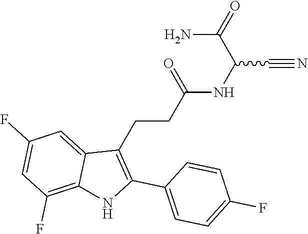 [SMILES: N#CC(NC(=O)CCc1c(-c2ccc(F)cc2)[nH]c2c(F)cc(F)cc12)C(N)=O] | 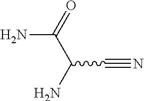 | LCMS m/z 401.12 [M + H]⁺ |
| 128 | As for Compound 91³ 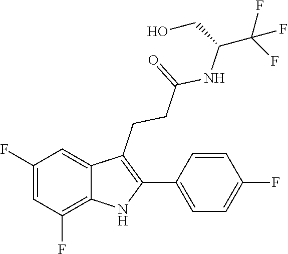 [SMILES: O=C(CCc1c(-c2ccc(F)cc2)[nH]c2c(F)cc(F)cc12)N[C@H](CO)C(F)(F)F] | 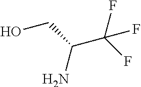 | ¹H NMR (300 MHz, Methanol-d₄) δ 7.74-7.48 (m, 2H), 7.33-7.05 (m, 3H), 6.72 (ddd, J = 11.1, 9.6, 2.2 Hz, 1H), 4.61 (tdd, J = 9.6, 5.5, 2.4 Hz, 1H), 3.84-3.70 (m, 1H), 3.64 (dd, J = 11.8, 6.6 Hz, 1H), 3.22- 3.03 (m, 2H), 2.75-2.53 (m, 2H); LCMS m/z 431.12 [M + H]⁺ |
| 129 | As for Compound 91³ 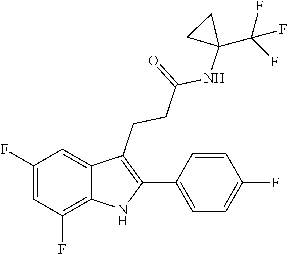 [SMILES: O=C(CCc1c(-c2ccc(F)cc2)[nH]c2c(F)cc(F)cc12)NC1(C(F)(F)F)CC1] | 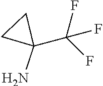 | LCMS m/z 427.12 [M + H]⁺ |
| 130 | As for Compound 91 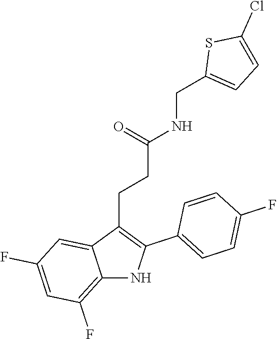 [SMILES: O=C(CCc1c(-c2ccc(F)cc2)[nH]c2c(F)cc(F)cc12)NCc1ccc(Cl)s1] | 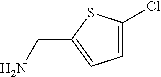 | LCMS m/z 448.65 [M + H]⁺ |
| 131 | As for Compound 91³ 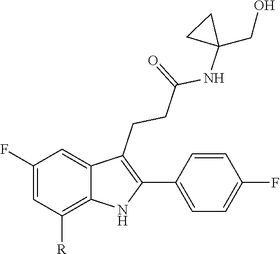 | 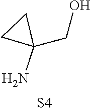 | LCMS m/z 389.17 [M + H]⁺ |
| 132 | As for Compound 91³ 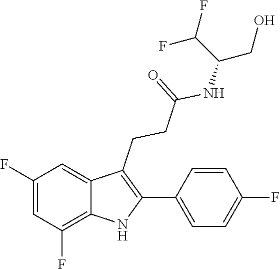 [SMILES: O=C(CCc1c(-c2ccc(F)cc2)[nH]c2c(F)cc(F)cc12)N[C@@H](CO)C(F)F] | 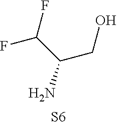 | ¹H NMR (300 MHz, Methanol-d₄) δ7.80-7.48 (m, 2H), 7.39-7.11 (m, 3H), 6.72 (ddd, J = 11.1, 9.6, 2.2 Hz, 1H), 5.88 (td, J = 55.6, 3.4 Hz, 1H), 4.24 (tdq, J = 12.0, 5.9, 3.0 Hz, 1H), 3.62 (q, J = 1.0 Hz, 1H), 3.23-3.07 (m, 2H), 2.68-2.49 (m, 2H); LCMS m/z 413.04 [M + H]⁺ |
| 133 | As for Compound 91³ 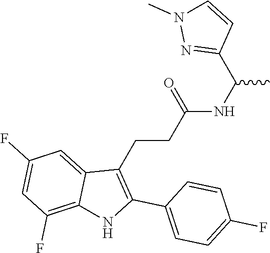 [SMILES: CC(NC(=O)CCc1c(-c2ccc(F)cc2)[nH]c2c(F)cc(F)cc12)c1ccn(C)n1] | 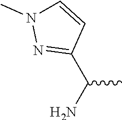 | LCMS m/z 427.18 [M + H]⁺ |
| 134 | As for Compound 91 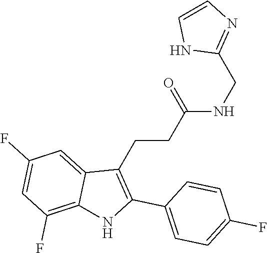 [SMILES: O=C(CCc1c(-c2ccc(F)cc2)[nH]c2c(F)cc(F)cc12)NCc1ncc[nH]1] | 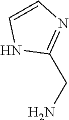 | ¹H NMR (300 MHz, Methanol-d₄) δ 8.20 (s, 2H), 7.77-7.58 (m, 2H), 7.31- 7.17 (m, 4H), 7.14 (dd, J = 9.5, 2.2 Hz, 1H), 6.73 (ddd, J = 11.0, 9.6, 2.2 Hz, 1H), 4.45 (s, 2H), 3.21-2.96 (m, 2H), 2.61-2.37 (m, 2H); LCMS m/z 399.17 [M + H]⁺ |
| 135 | As for Compound 91 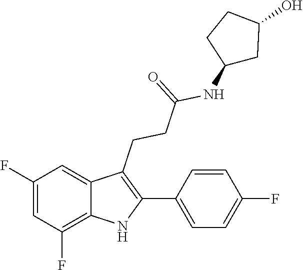 [SMILES: O=C(CCc1c(-c2ccc(F)cc2)[nH]c2c(F)cc(F)cc12)N[C@H]1CC[C@H](O)C1] | 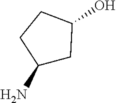 | LCMS m/z 403.17 [M + H]⁺ |
| 136 | As for Compound 91 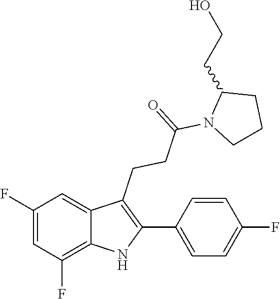 [SMILES: O=C(CCc1c(-c2ccc(F)cc2)[nH]c2c(F)cc(F)cc12)N1CCCC1CCO] | 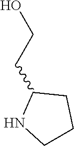 | LCMS m/z 417.18 [M + H]⁺ |
| 137 | As for Compound 91 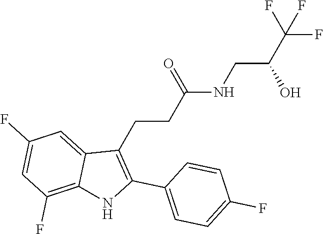 [SMILES: O=C(CCc1c(-c2ccc(F)cc2)[nH]c2c(F)cc(F)cc12)NC[C@@H](O)C(F)(F)F] | 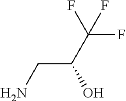 | LCMS m/z 431.09 [M + H]⁺ |
| 138 | As for Compound 91 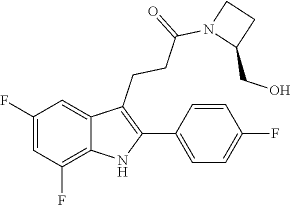 [SMILES: O=C(CCc1c(-c2ccc(F)cc2)[nH]c2c(F)cc(F)cc12)N1CC[C@H]1CO] | 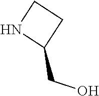 | LCMS m/z 389.13 [M + H]⁺ |
| 139 | As for Compound 91³ 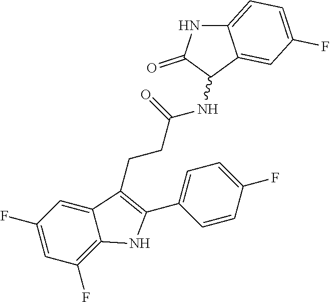 [SMILES: O=C(CCc1c(-c2ccc(F)cc2)[nH]c2c(F)cc(F)cc12)NC1C(=O)Nc2ccc(F)cc21] | 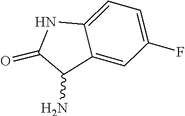 [SMILES: NC1C(=O)Nc2ccc(F)cc21] | LCMS m/z 468.13 [M + H]⁺ |
| 140 | As for Compound 91 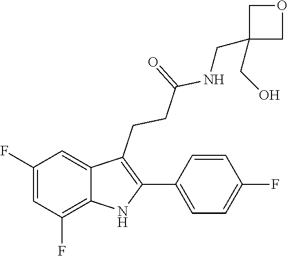 [SMILES: O=C(CCc1c(-c2ccc(F)cc2)[nH]c2c(F)cc(F)cc12)NCC1(CO)COC1] | 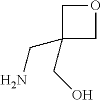 | LCMS m/z 419.13 [M + H]⁺ |
| 141 | As for Compound 91³ 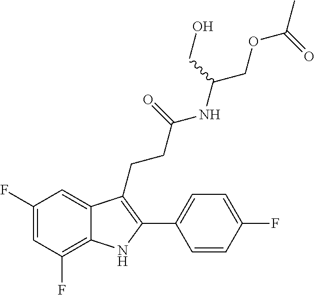 [SMILES: CC(=O)OCC(CO)NC(=O)CCc1c(-c2ccc(F)cc2)[nH]c2c(F)cc(F)cc12] | 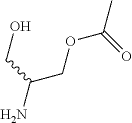 | LCMS m/z 435.13 [M + H]⁺ |
| 142 | As for Compound 91³ 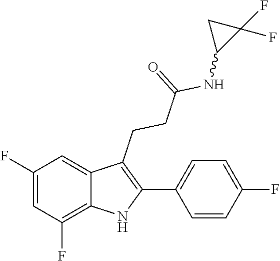 [SMILES: O=C(CCc1c(-c2ccc(F)cc2)[nH]c2c(F)cc(F)cc12)NC1CC1(F)F] | 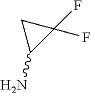 | LCMS m/z 395.13 [M + H]⁺ |
| 143 | As for Compound 91³ 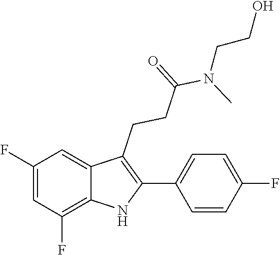 [SMILES: CN(CCO)C(=O)CCc1c(-c2ccc(F)cc2)[nH]c2c(F)cc(F)cc12] | 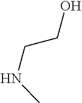 | LCMS m/z 377.18 [M + H]⁺ |
| 144 | As for Compound 92 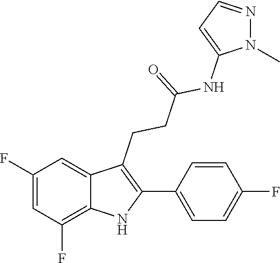 [SMILES: Cn1nccc1NC(=O)CCc1c(-c2ccc(F)cc2)[nH]c2c(F)cc(F)cc12] | 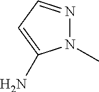 | LCMS m/z 399.17 [M + H]⁺ |
| 145 | As for Compound 91 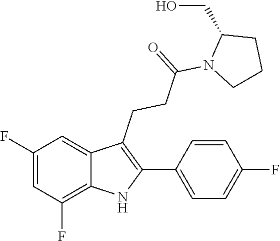 [SMILES: O=C(CCc1c(-c2ccc(F)cc2)[nH]c2c(F)cc(F)cc12)N1CCC[C@H]1CO] | 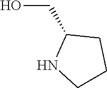 | LCMS m/z 403.14 [M + H]⁺ |
| 146 | As for Compound 92 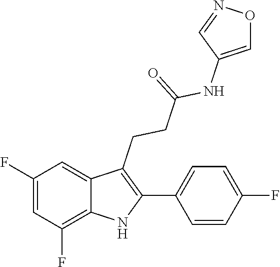 [SMILES: O=C(CCc1c(-c2ccc(F)cc2)[nH]c2c(F)cc(F)cc12)Nc1cnoc1] | 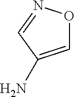 | LCMS m/z 386.11 [M + H]⁺ |
| 147 | As for Compound 91 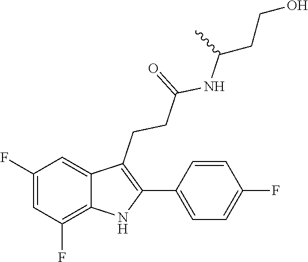 [SMILES: CC(CCO)NC(=O)CCc1c(-c2ccc(F)cc2)[nH]c2c(F)cc(F)cc12] | 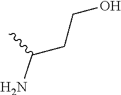 | LCMS m/z 391.15 [M + H]⁺ |
| 148 | As for Compound 92 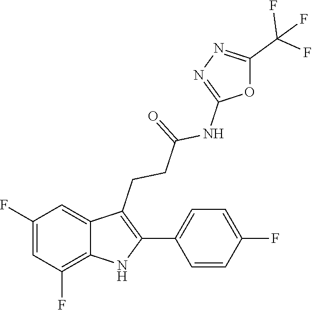 [SMILES: O=C(CCc1c(-c2ccc(F)cc2)[nH]c2c(F)cc(F)cc12)Nc1nnc(C(F)(F)F)o1] | 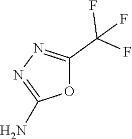 | LCMS m/z 455.19 [M + H]⁺ |
| 149 | As for Compound 91 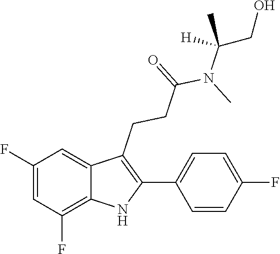 [SMILES: C[C@@H](CO)N(C)C(=O)CCc1c(-c2ccc(F)cc2)[nH]c2c(F)cc(F)cc12] | 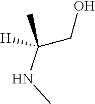 | ¹H NMR (300 MHz, Methanol-d₄) δ 7.68-7.62 (m, 2H), 7.26 (t, J = 8.8 Hz, 2H), 7.16 (dt, J = 9.3, 1.8 Hz, 1H), 6.87-6.71 (m, 1H), 4.76-4.56 (m, 1H), 3.98-3.84 (m, 1H), 3.58- 3.40 (m, 2H), 3.15 (t, J = 7.9 Hz, 3H), 2.75 (d, J = 4.1 Hz, 4H), 1.05-0.65 (m, 3H); LCMS m/z 391.15 [M + H]⁺ |
| 150 | As for Compound 91³ 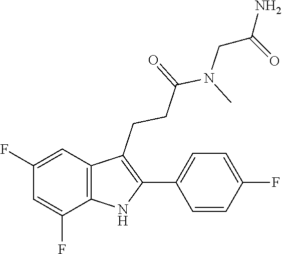 [SMILES: CN(CC(N)=O)C(=O)CCc1c(-c2ccc(F)cc2)[nH]c2c(F)cc(F)cc12] | 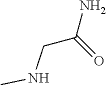 | ¹H NMR (300 MHz, Methanol-d₄) δ 7.71-7.51 (m, 2H), 7.31-7.17 (m, 2H), 7.12 (ddd, J = 13.0, 9.4, 2.2 Hz, 1H), 6.73 (dddd, J = 12.8, 9.6, 3.1, 2.2 Hz, 1H), 3.94 (2s, 2H), 3.12 (ddd, J = 9.2, 7.4, 3.3 Hz, 2H), 2.92 (2s, 3H), 2.83- 2.53 (m, 2H); LCMS m/z 390.14 [M + H]⁺ |
| 151 | As for Compound 92 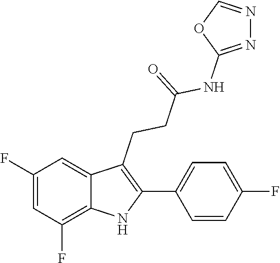 [SMILES: O=C(CCc1c(-c2ccc(F)cc2)[nH]c2c(F)cc(F)cc12)Nc1nnco1] | 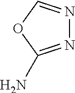 | LCMS m/z 387.11 [M + H]⁺ |
| 152 | As for Compound 91³ 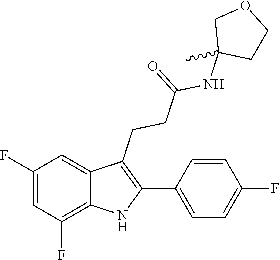 [SMILES: CC1(NC(=O)CCc2c(-c3ccc(F)cc3)[nH]c3c(F)cc(F)cc23)CCOC1] | 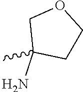 | LCMS m/z 403.17 [M + H]⁺ |
| 153 | As for Compound 91 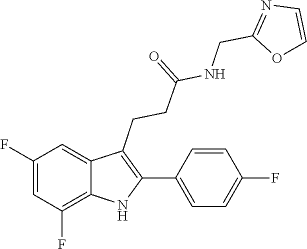 [SMILES: O=C(CCc1c(-c2ccc(F)cc2)[nH]c2c(F)cc(F)cc12)NCc1ncco1] | 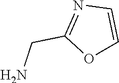 | LCMS m/z 400.14 [M + H]⁺ |
| 154 | As for Compound 91 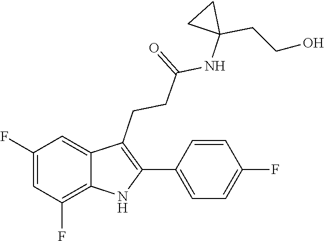 [SMILES: O=C(CCc1c(-c2ccc(F)cc2)[nH]c2c(F)cc(F)cc12)NC1(CCO)CC1] | 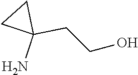 | LCMS m/z 403.14 [M + H]⁺ |
| 155 | As for Compound 91 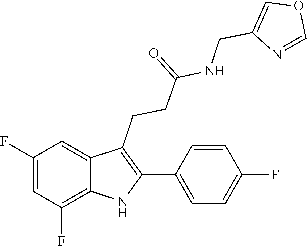 [SMILES: O=C(CCc1c(-c2ccc(F)cc2)[nH]c2c(F)cc(F)cc12)NCc1cocn1] | 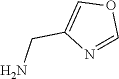 | LCMS m/z 400.14 [M + H]⁺ |
| 156 | As for Compound 91³ 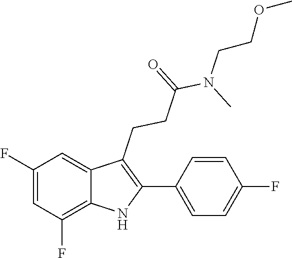 [SMILES: COCCN(C)C(=O)CCc1c(-c2ccc(F)cc2)[nH]c2c(F)cc(F)cc12] | 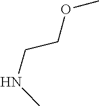 | LCMS m/z 391.19 [M + H]⁺ |
| 157 | As for Compound 91³ 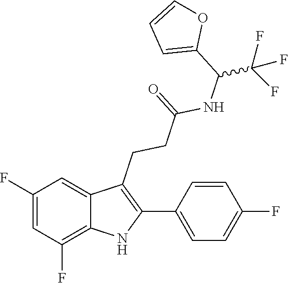 [SMILES: O=C(CCc1c(-c2ccc(F)cc2)[nH]c2c(F)cc(F)cc12)NC(c1ccco1)C(F)(F)F] | 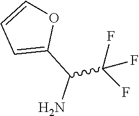 [SMILES: NC(c1ccco1)C(F)(F)F] | LCMS m/z 467.08 [M + H]⁺ |
| 158 | As for Compound 91³ 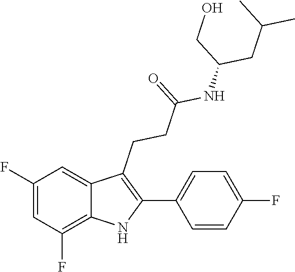 [SMILES: CC(C)C[C@@H](CO)NC(=O)CCc1c(-c2ccc(F)cc2)[nH]c2c(F)cc(F)cc12] | 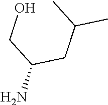 | LCMS m/z 419.2 [M + H]⁺ |
| 159 | As for Compound 91 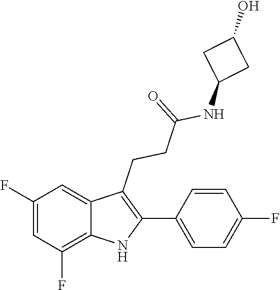 [SMILES: O=C(CCc1c(-c2ccc(F)cc2)[nH]c2c(F)cc(F)cc12)N[C@H]1C[C@H](O)C1] | 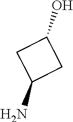 | LCMS m/z 389 [M + H]⁺ |
| 160 | As for Compound 92 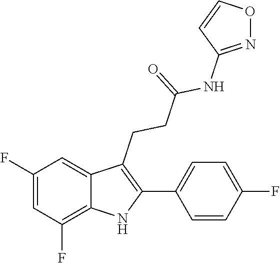 [SMILES: O=C(CCc1c(-c2ccc(F)cc2)[nH]c2c(F)cc(F)cc12)Nc1ccon1] | 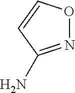 | LCMS m/z 386.04 [M + H]⁺ |
| 161 | As for Compound 91³ 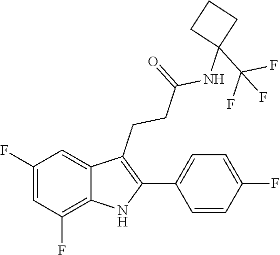 [SMILES: O=C(CCc1c(-c2ccc(F)cc2)[nH]c2c(F)cc(F)cc12)NC1(C(F)(F)F)CCC1] | 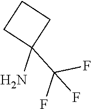 | LCMS m/z 441.12 [M + H]⁺ |
| 162 | As for Compound 92 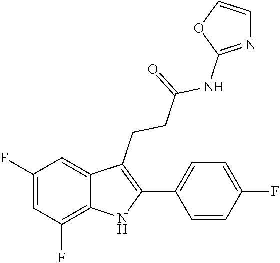 [SMILES: O=C(CCc1c(-c2ccc(F)cc2)[nH]c2c(F)cc(F)cc12)Nc1ncco1] | 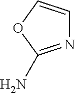 | LCMS m/z 386.11 [M + H]⁺ |
| 163 | As for Compound 92 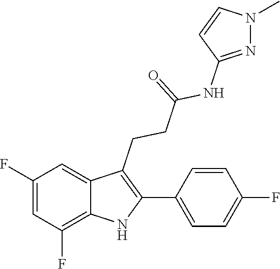 [SMILES: Cn1ccc(NC(=O)CCc2c(-c3ccc(F)cc3)[nH]c3c(F)cc(F)cc23)n1] | 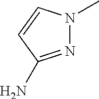 | ¹H NMR (300 MHz, Methanol-d₄) δ 7.71-7.56 (m, 2H), 7.41 (d, J = 2.3 Hz, 1H), 7.29-7.11 (m, 3H), 6.72 (ddd, J = 11.1, 9.6, 2.2 Hz, 1H), 6.45 (d, J = 2.3 Hz, 1H), 3.76 (s, 3H), 3.26- 3.04 (m, 2H), 2.81-2.57 (m, 2H); LCMS m/z 399.17 [M + H]⁺ |
| 164 | As for Compound 91³ 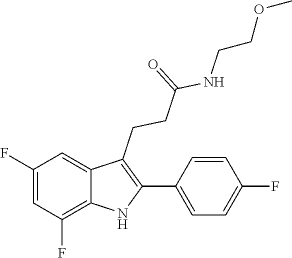 [SMILES: COCCNC(=O)CCc1c(-c2ccc(F)cc2)[nH]c2c(F)cc(F)cc12] | 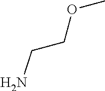 | LCMS m/z 377.18 [M + H]⁺ |
| 165 | As for Compound 92 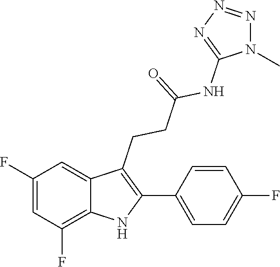 [SMILES: Cn1nnnc1NC(=O)CCc1c(-c2ccc(F)cc2)[nH]c2c(F)cc(F)cc12] | 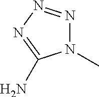 | LCMS m/z 401.15 [M + H]⁺ |
| 166 | As for Compound 91³ 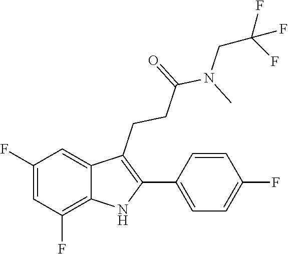 [SMILES: CN(CC(F)(F)F)C(=O)CCc1c(-c2ccc(F)cc2)[nH]c2c(F)cc(F)cc12] | 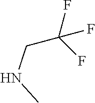 | LCMS m/z 415.13 [M + H]⁺ |
| 167 | As for Compound 92 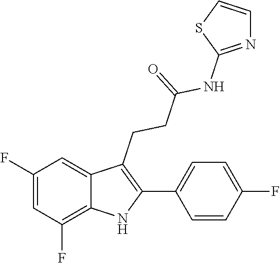 [SMILES: O=C(CCc1c(-c2ccc(F)cc2)[nH]c2c(F)cc(F)cc12)Nc1nccs1] | 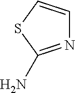 | ¹H NMR (300 MHz, Methanol-d₄) δ 7.74-7.53 (m, 2H), 7.37 (d, J = 3.6 Hz, 1H), 7.27-7.11 (m, 3H), 7.07 (d, J = 3.6 Hz, 1H), 6.71 (ddd, J = 11.1, 9.6, 2.2 Hz, 1H), 3.23 (dd, J = 8.4, 6.8 Hz, 2H), 2.77 (dd, J = 8.4, 6.8 Hz, 2H); LCMS m/z 402.23 [M + H]⁺ |
| 168 | As for Compound 91 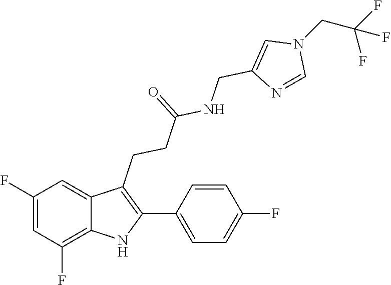 [SMILES: O=C(CCc1c(-c2ccc(F)cc2)[nH]c2c(F)cc(F)cc12)NCc1cn(CC(F)(F)F)cn1] | 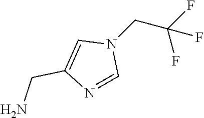 [SMILES: NCc1cn(CC(F)(F)F)cn1] | LCMS m/z 480.7 [M + H]⁺ |
| 169 | As for Compound 91³ 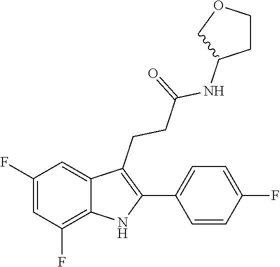 [SMILES: O=C(CCc1c(-c2ccc(F)cc2)[nH]c2c(F)cc(F)cc12)NC1CCOC1] | 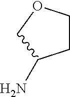 | ¹H NMR (300 MHz, Methanol-d₄) δ 7.72-7.51 (m, 2H), 7.29-7.07 (m, 3H), 6.73 (ddd, J = 11.0, 9.6, 2.2 Hz, 1H), 4.27 (ddd, J = 9.7, 7.5, 3.8 Hz, 1H), 3.84-3.61 (m, 3H), 3.41 (dd, J = 9.1, 3.6 Hz, 1H), 3.11 (dd, J = 8.3, 6.9 Hz, 2H), 2.49 (dd, J = 8.2, 7.0 Hz, 2H), 2.10 (dq, J = 13.0, 7.6 Hz, 1H), 1.65 (dddd, J = 13.1, 7.4, 5.7, 3.8 Hz, 1H); LCMS m/z 389.18 [M + H]⁺ |
| 170 | As for Compound 91³ 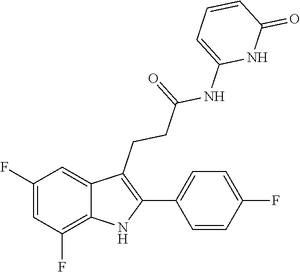 [SMILES: O=C(CCc1c(-c2ccc(F)cc2)[nH]c2c(F)cc(F)cc12)Nc1cccc(=O)[nH]1] | 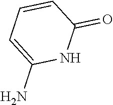 | LCMS m/z 412.13 [M + H]⁺ |
| 171 | As for Compound 92 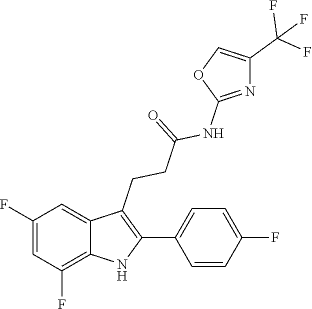 [SMILES: O=C(CCc1c(-c2ccc(F)cc2)[nH]c2c(F)cc(F)cc12)Nc1nc(C(F)(F)F)co1] | 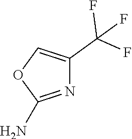 | LCMS m/z 454.05 [M + H]⁺ |
| 172 | As for Compound 92 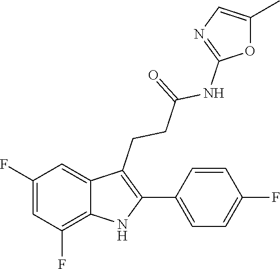 [SMILES: Cc1cnc(NC(=O)CCc2c(-c3ccc(F)cc3)[nH]c3c(F)cc(F)cc23)o1] | 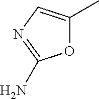 | LCMS m/z 400.14 [M + H]⁺ |
| 173 | As for Compound 91⁸ 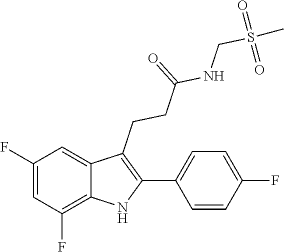 [SMILES: CS(=O)(=O)CNC(=O)CCc1c(-c2ccc(F)cc2)[nH]c2c(F)cc(F)cc12] | 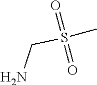 | LCMS m/z 411.02 [M + H]⁺ |
| 174 | As for Compound 91³ 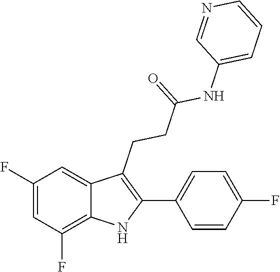 [SMILES: O=C(CCc1c(-c2ccc(F)cc2)[nH]c2c(F)cc(F)cc12)Nc1cccnc1] | 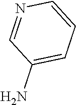 | ¹H NMR (300 MHz, Methanol-d₄) δ 8.65 (d, J = 2.5 Hz, 1H), 8.25 (dd, J = 4.9, 1.5 Hz, 1H), 8.03 (ddd, J = 8.3, 2.5, 1.4 Hz, 1H), 7.76-7.57 (m, 2H), 7.40 (ddd, J = 8.4, 4.9, 0.8 Hz, 1H), 7.31-7.10 (m, 3H), 6.74 (ddd, J = 11.1, 9.6, 2.2 Hz, 1H), 3.26 (t, J = 7.6 Hz, 2H), 2.85-2.45 (m, 2H); LCMS m/z 396.11 [M + H]⁺ |
| 175 | As for Compound 91³ 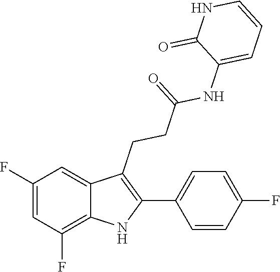 [SMILES: O=C(CCc1c(-c2ccc(F)cc2)[nH]c2c(F)cc(F)cc12)Nc1ccc[nH]c1=O] | 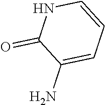 | LCMS m/z 412.13 [M + H]⁺ |
| 176 | As for Compound 91³ 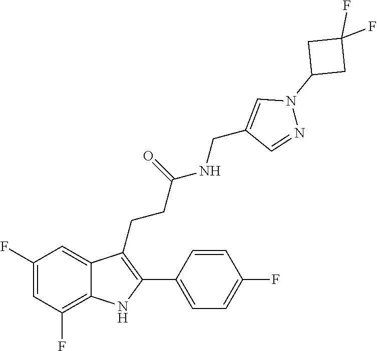 [SMILES: O=C(CCc1c(-c2ccc(F)cc2)[nH]c2c(F)cc(F)cc12)NCc1cnn(C2CC(F)(F)C2)c1] | 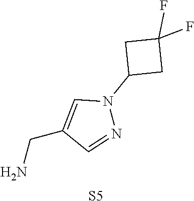 | LCMS m/z 489.17 [M + H]⁺ |
| 177 | As for Compound 91 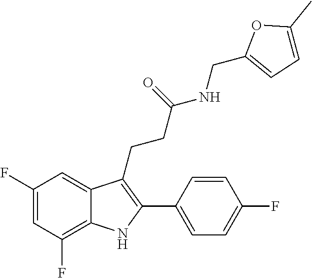 [SMILES: Cc1ccc(CNC(=O)CCc2c(-c3ccc(F)cc3)[nH]c3c(F)cc(F)cc23)o1] | 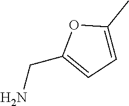 | ¹H NMR (300 MHz, Methanol-d₄) δ 7.70-7.56 (m, 2H), 7.31-7.06 (m, 3H), 6.72 (ddd, J = 11.1, 9.6, 2.2 Hz, 1H), 5.95 (d, J = 3.1 Hz, 1H), 5.86 (dq, J = 3.2, 1.1 Hz, 1H), 4.20 (s, 2H), 3.11 (dd, J = 8.7, 6.8 Hz, 2H), 2.52 (dd, J = 8.7, 6.8 Hz, 2H), 2.19 (dd, J = 1.0, 0.5 Hz, 3H); LCMS m/z 413.14 [M + H]⁺ |
| 178 | As for Compound 92 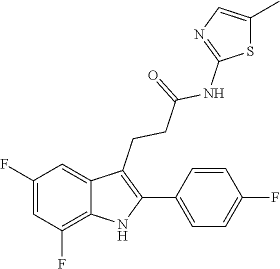 [SMILES: Cc1cnc(NC(=O)CCc2c(-c3ccc(F)cc3)[nH]c3c(F)cc(F)cc23)s1] | 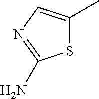 | ¹H NMR (300 MHz, Methanol-d₄) δ 7.61 (dd, J = 8.5, 5.3 Hz, 2H), 7.17 (q, J = 8.6 Hz, 3H), 7.03 (s, 1H), 6.72 (t, J = 10.6 Hz, 1H), 3.22 (t, J = 7.7 Hz, 2H), 2.75 (t, J = 7.6 Hz, 2H), 2.37 (s, 3H); LCMS m/z 416.11 [M + H]⁺ |
| 179 | As for Compound 91 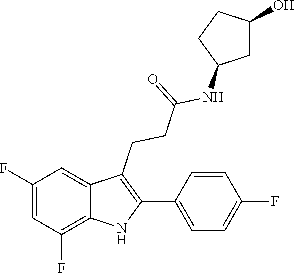 [SMILES: O=C(CCc1c(-c2ccc(F)cc2)[nH]c2c(F)cc(F)cc12)N[C@H]1CC[C@@H](O)C1] | 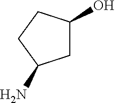 | LCMS m/z 403.14 [M + H]⁺ |
| 180 | As for compound 92 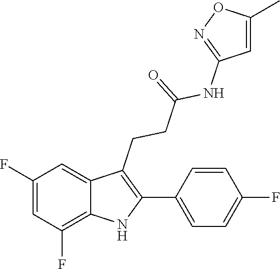 [SMILES: Cc1cc(NC(=O)CCc2c(-c3ccc(F)cc3)[nH]c3c(F)cc(F)cc23)no1] | 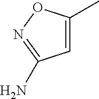 | LCMS m/z 400.14 [M + H]⁺ |
| 181 | As for Compound 91 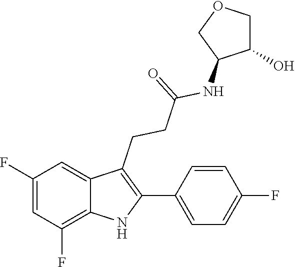 [SMILES: O=C(CCc1c(-c2ccc(F)cc2)[nH]c2c(F)cc(F)cc12)N[C@H]1COC[C@@H]1O] | 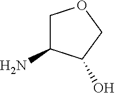 | LCMS m/z 405.12 [M + H]⁺ |
| 182 | As for Compound 91³ 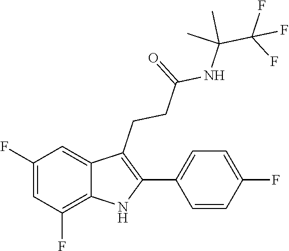 [SMILES: CC(C)(NC(=O)CCc1c(-c2ccc(F)cc2)[nH]c2c(F)cc(F)cc12)C(F)(F)F] | 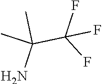 | LCMS m/z 429.14 [M + H]⁺ |
| 183 | As for Compound 91³ 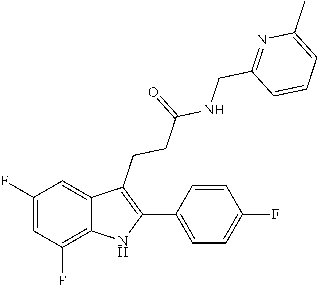 [SMILES: Cc1cccc(CNC(=O)CCc2c(-c3ccc(F)cc3)[nH]c3c(F)cc(F)cc23)n1] | 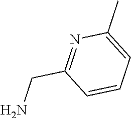 | ¹H NMR (300 MHz, Methanol-d₄) δ 7.74-7.55 (m, 3H), 7.30-7.08 (m, 4H), 6.83-6.76 (m, 1H), 6.77-6.67 (m, 1H), 4.37 (s, 2H), 3.19 (t, J = 7.5 Hz, 2H), 2.75-2.55 (m, 2H), 2.51 (s, 3H); LCMS m/z 424.18 [M + H]⁺ |
| 184 | As for Compound 92 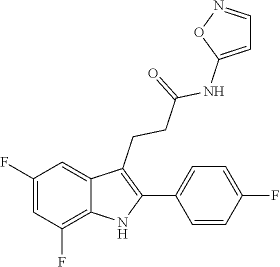 [SMILES: O=C(CCc1c(-c2ccc(F)cc2)[nH]c2c(F)cc(F)cc12)Nc1ccno1] | 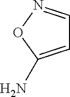 | LCMS m/z 386.14 [M + H]⁺ |
| 185 | As for Compound 91³ 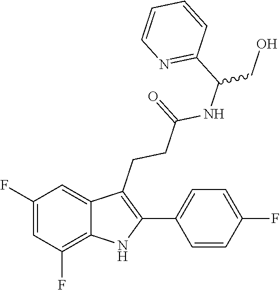 [SMILES: O=C(CCc1c(-c2ccc(F)cc2)[nH]c2c(F)cc(F)cc12)NC(CO)c1ccccn1] | 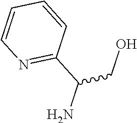 | ¹H NMR (300 MHz, Methanol-d₄) δ 8.47 (ddd, J = 5.0, 1.8, 0.9 Hz, 1H), 7.73 (td, J = 7.7, 1.8 Hz, 1H), 7.67-7.57 (m, 2H), 7.40- 7.08 (m, 6H), 6.73 (ddd, J = 11.5, 9.6, 2.2 Hz, 1H), 5.01 (t, J = 5.8 Hz, 1H), 3.76 (dd, J = 5.8, 2.5 Hz, 2H), 3.22- 3.05 (m, 2H), 2.75-2.58 (m, 2H); LCMS m/z 440.15 [M + H]⁺ |
| 186 | As for Compound 91 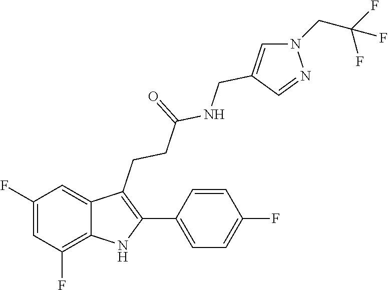 [SMILES: O=C(CCc1c(-c2ccc(F)cc2)[nH]c2c(F)cc(F)cc12)NCc1cnn(CC(F)(F)F)c1] | 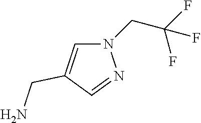 [SMILES: NCc1cnn(CC(F)(F)F)c1] | LCMS m/z 481.15 [M + H]⁺ |
| 187 | As for Compound 91 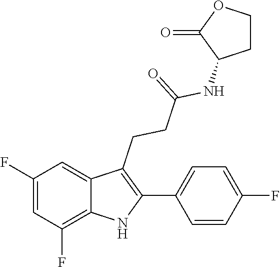 [SMILES: O=C(CCc1c(-c2ccc(F)cc2)[nH]c2c(F)cc(F)cc12)N[C@H]1CCOC1=O] | 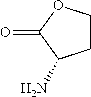 | LCMS m/z 403.14 [M + H]⁺ |
| 188 | As for Compound 91 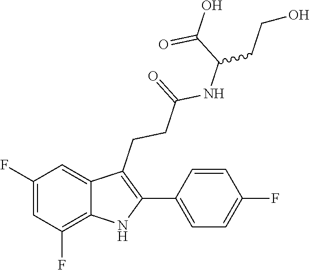 [SMILES: O=C(CCc1c(-c2ccc(F)cc2)[nH]c2c(F)cc(F)cc12)NC(CCO)C(=O)O] | 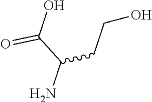 | LCMS m/z 420.63 [M + H]⁺ |
| 189 | As for Compound 91 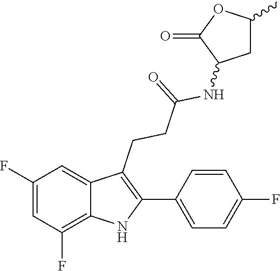 [SMILES: CC1CC(NC(=O)CCc2c(-c3ccc(F)cc3)[nH]c3c(F)cc(F)cc23)C(=O)O1] | 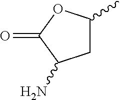 | LCMS m/z 416.76 [M + H]⁺ |
| 190 | As for Compound 91³ 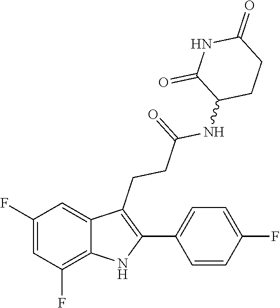 [SMILES: O=C1CCC(NC(=O)CCc2c(-c3ccc(F)cc3)[nH]c3c(F)cc(F)cc23)C(=O)N1] | 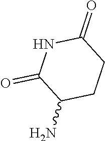 | LCMS m/z 430.15 [M + H]⁺ |
| 191 | As for Compound 91 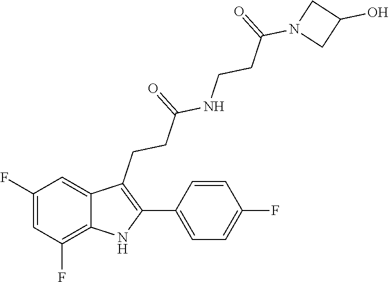 [SMILES: O=C(CCc1c(-c2ccc(F)cc2)[nH]c2c(F)cc(F)cc12)NCCC(=O)N1CC(O)C1] | 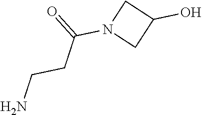 | LCMS m/z 446.14 [M + H]⁺ |
| 192 | As for Compound 91³ 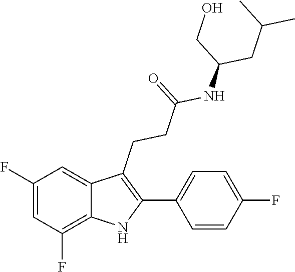 [SMILES: CC(C)C[C@H](CO)NC(=O)CCc1c(-c2ccc(F)cc2)[nH]c2c(F)cc(F)cc12] | 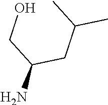 | LCMS m/z 419.2 [M + H]⁺ |
| 193 | As for Compound 91 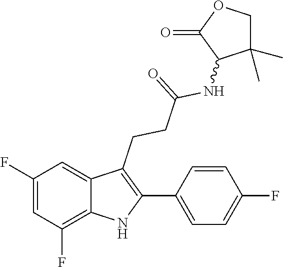 [SMILES: CC1(C)COC(=O)C1NC(=O)CCc1c(-c2ccc(F)cc2)[nH]c2c(F)cc(F)cc12] | 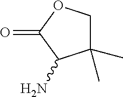 | LCMS m/z 430.99 [M + H]⁺ |

¹DIPEA was used as a base
²DMF was used as solvent
³Stirred for 12 hours
⁴Stirred at 40° C. for 10 minutes
⁵Diluted with EtOAc, washed with water and brine, dried with magnesium sulfate and filtered.
⁶Stirred with DCM for 20 minutes while precipitation occurred. Solid filtered and rinsed with ether.
⁷Stirred overnight.
⁸Amine was Boc protected. Stirred in TFA (1 mL) for 30 minutes. Evaporated to dryness and used in the amidation step without further purification.

### Compound 194

### 3-[5-chloro-7-fluoro-2-(4-fluorophenyl)-1H-indol-3-yl]-N-[1-(hydroxymethyl)cyclobutyl]propanamide (194)

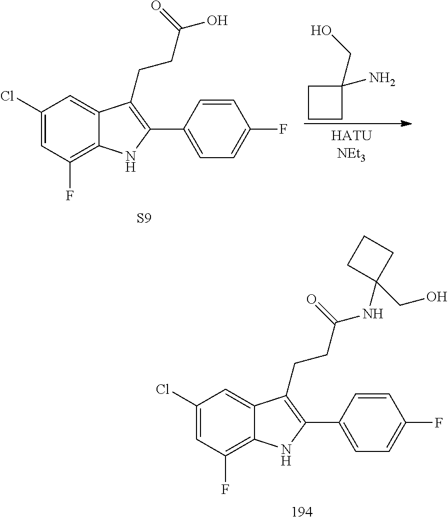

### Preparation of 3-[5-chloro-7-fluoro-2-(4-fluorophenyl)-1H-indol-3-yl]-N-[1-(hydroxymethyl)-cyclobutyl]propanamide (194)

To 3-[5-chloro-7-fluoro-2-(4-fluorophenyl)-1H-indol-3-yl]propanoic acid S9 (12 mg, 0.03288 mmol), HATU (20 mg, 0.05260 mmol) and (1-aminocyclobutyl)methanol (7 mg, 0.06921 mmol) in DMSO (0.5 mL) was added TEA (30 μL, 0.2152 mmol). The reaction was stirred at room temperature for 12 hours. The crude material was purified by reversed-phase HPLC (Method: C18 Waters Sunfire column (30×150 mm, 5 micron). Gradient: MeCN in H₂O with 0.1% trifluoroacetic acid) to afford 3-[5-chloro-7-fluoro-2-(4-fluorophenyl)-1H-indol-3-yl]-N-[1-(hydroxymethyl)cyclobutyl]propanamide 194 (11 mg, 80%). LCMS m/z 418.97 [M+H]⁺.

### Compounds 195-212

Compounds 195-212 (see Table 4) were prepared in a single step from intermediate S9 using the method described for the preparation of compound 194. Amines were prepared by methods described above or obtained from commercial sources. Any modifications to methods are noted in Table 4.

TABLE 4
Structure and physicochemical data for compounds 195-212

| | | | ¹H NMR; LCMS |
|---|---|---|---|
| Compound | Product | Amine Reagent | m/z [M + H]⁺ |
| 195 | 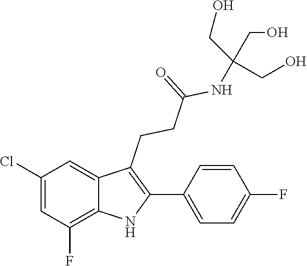 [SMILES: O=C(CCc1c(-c2ccc(F)cc2)[nH]c2c(F)cc(Cl)cc12)NC(CO)(CO)CO] | 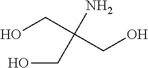 | LCMS m/z 439.1 [M + H]⁺ |
| 196 | 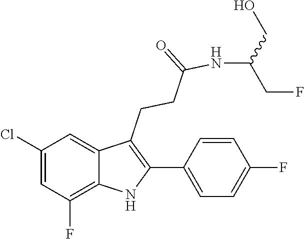 [SMILES: O=C(CCc1c(-c2ccc(F)cc2)[nH]c2c(F)cc(Cl)cc12)NC(CO)CF] | 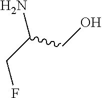 | LCMS m/z 411.12 [M + H]⁺ |
| 197 | 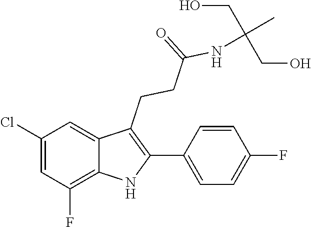 [SMILES: CC(CO)(CO)NC(=O)CCc1c(-c2ccc(F)cc2)[nH]c2c(F)cc(Cl)cc12] | 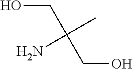 | LCMS m/z 423.14 [M + H]⁺ |
| 198 | 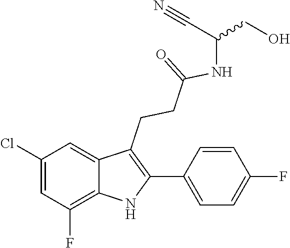 [SMILES: N#CC(CO)NC(=O)CCc1c(-c2ccc(F)cc2)[nH]c2c(F)cc(Cl)cc12] | 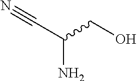 | LCMS m/z 404.24 [M + H]⁺ |
| 199 | 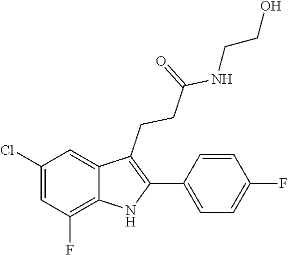 [SMILES: O=C(CCc1c(-c2ccc(F)cc2)[nH]c2c(F)cc(Cl)cc12)NCCO] | 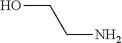 | LCMS m/z 379.09 [M + H]⁺ |
| 200 | 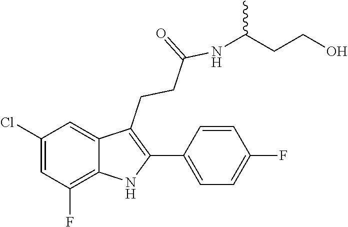 [SMILES: CC(CCO)NC(=O)CCc1c(-c2ccc(F)cc2)[nH]c2c(F)cc(Cl)cc12] | 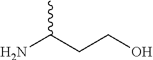 | LCMS m/z 407.11 [M + H]⁺ |
| 201 | 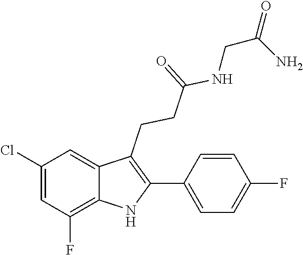 [SMILES: NC(=O)CNC(=O)CCc1c(-c2ccc(F)cc2)[nH]c2c(F)cc(Cl)cc12] | 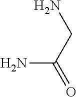 | LCMS m/z 392.09 [M + H]⁺ |
| 202 | 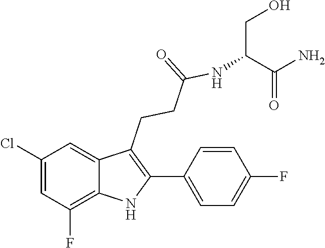 [SMILES: NC(=O)[C@@H](CO)NC(=O)CCc1c(-c2ccc(F)cc2)[nH]c2c(F)cc(Cl)cc12] | 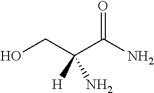 | LCMS m/z 422.16 [M + H]⁺ |
| 203 | 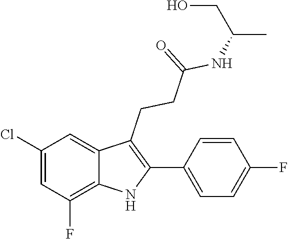 [SMILES: C[C@@H](CO)NC(=O)CCc1c(-c2ccc(F)cc2)[nH]c2c(F)cc(Cl)cc12] | 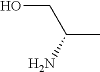 | LCMS m/z 393.13 [M + H]⁺ |
| 204 | 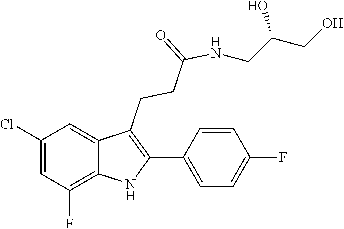 [SMILES: O=C(CCc1c(-c2ccc(F)cc2)[nH]c2c(F)cc(Cl)cc12)NC[C@H](O)CO] | 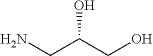 | LCMS m/z 409.03 [M + H]⁺ |
| 205 | 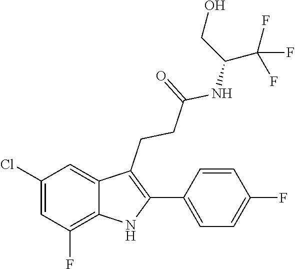 [SMILES: O=C(CCc1c(-c2ccc(F)cc2)[nH]c2c(F)cc(Cl)cc12)N[C@H](CO)C(F)(F)F] | 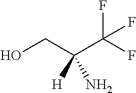 | LCMS m/z 447.21 [M + H]⁺ |
| 206 | 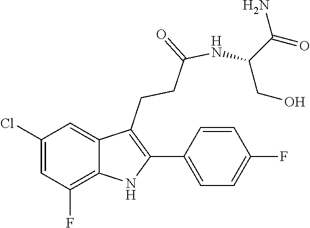 [SMILES: NC(=O)[C@H](CO)NC(=O)CCc1c(-c2ccc(F)cc2)[nH]c2c(F)cc(Cl)cc12] | 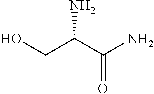 | LCMS m/z 422.26 [M + H]⁺ |
| 207 | 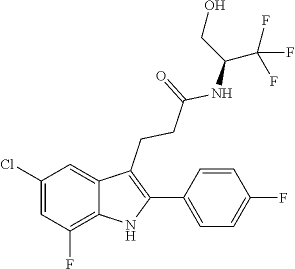 [SMILES: O=C(CCc1c(-c2ccc(F)cc2)[nH]c2c(F)cc(Cl)cc12)N[C@@H](CO)C(F)(F)F] | 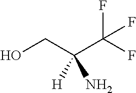 | LCMS m/z 446.92 [M + H]⁺ |
| 208 | 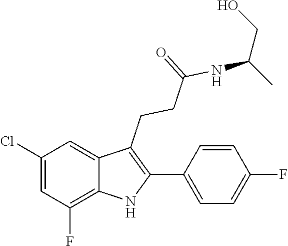 [SMILES: C[C@H](CO)NC(=O)CCc1c(-c2ccc(F)cc2)[nH]c2c(F)cc(Cl)cc12] | 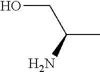 | LCMS m/z 393.1 [M + H]⁺ |
| 209 | 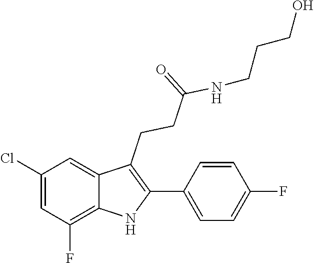 [SMILES: O=C(CCc1c(-c2ccc(F)cc2)[nH]c2c(F)cc(Cl)cc12)NCCCO] | 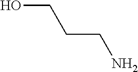 | LCMS m/z 393.1 [M + H]⁺ |
| 210 | 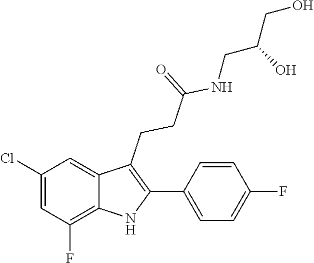 [SMILES: O=C(CCc1c(-c2ccc(F)cc2)[nH]c2c(F)cc(Cl)cc12)NC[C@@H](O)CO] | 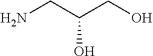 | LCMS m/z 408.93 [M + H]⁺ |
| 211 | 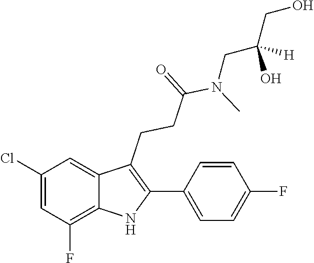 [SMILES: CN(C[C@H](O)CO)C(=O)CCc1c(-c2ccc(F)cc2)[nH]c2c(F)cc(Cl)cc12] | 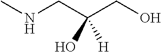 | LCMS m/z 423.14 [M + H]⁺ |
| 212 | 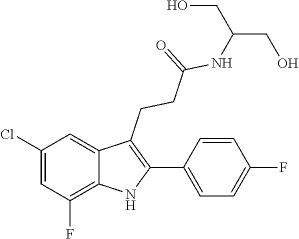 [SMILES: O=C(CCc1c(-c2ccc(F)cc2)[nH]c2c(F)cc(Cl)cc12)NC(CO)CO] | 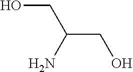 | LCMS m/z 409.13 [M + H]⁺ |

### Compound 213

### 3-[7-fluoro-2-(4-fluorophenyl)-5-methyl-1H-indol-3-yl]-N-[(1R)-2-hydroxy-1-methyl-ethyl]propenamide (213)

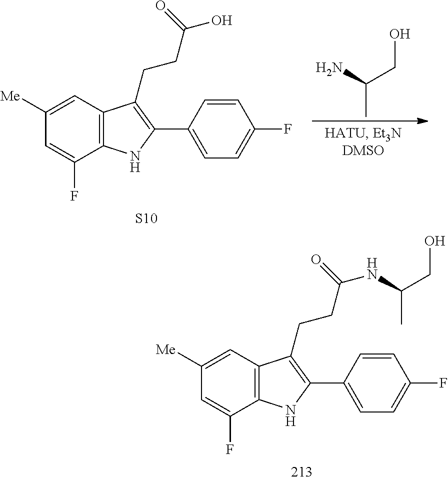

### Preparation of 3-[7-fluoro-2-(4-fluorophenyl)-5-methyl-1H-indol-3-yl]-N-[(1R)-2-hydroxy-1-methyl-ethyl]propenamide (213)

To a solution of 3-[7-fluoro-2-(4-fluorophenyl)-5-methyl-1H-indol-3-yl]propanoic acid S10 (12 mg, 0.036 mmol) and (2R)-2-aminopropan-1-ol (5 mg, 0.067 mmol) in DMSO (0.5 mL) was added HATU (25 mg, 0.066 mmol) and Et₃N (30 μL, 0.22 mmol). The reaction was stirred at ambient temperature for 12 hours. The mixture was then purified by reversed-phase HPLC (Method: C18 Waters Sunfire column (30×150 mm, 5 micron). Gradient: MeCN in H₂O with 0.1% trifluoroacetic acid) to afford the (6.6 mg, 49%). LCMS m/z 373.2 [M+H]⁺.

### Compounds 214-226

Compounds 214-226 (see Table 5) were prepared in a single step from intermediate S10 using the appropriate reagents and the amide coupling method as described for compound 213. Amines were prepared by methods described above or obtained from commercial sources. Any modifications to methods are noted in Table 5 and accompanying footnotes.

TABLE 5
Method of preparation, structure and physicochemical data for compounds 214-226

| | | Amine | ¹H NMR; LCMS m/z |
|---|---|---|---|
| Compound | Product | Reagent | [M + H]⁺ |
| 214 | 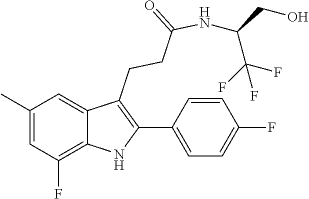 [SMILES: Cc1cc(F)c2[nH]c(-c3ccc(F)cc3)c(CCC(=O)N[C@@H](CO)C(F)(F)F)c2c1] | 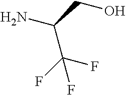 | LCMS m/z 426.95 [M + H]⁺ |
| 215 | 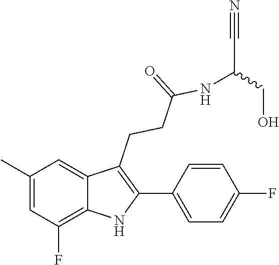 [SMILES: Cc1cc(F)c2[nH]c(-c3ccc(F)cc3)c(CCC(=O)NC(C#N)CO)c2c1] | 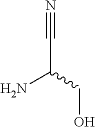 | LCMS m/z 384.14 [M + H]⁺ |
| 216 | 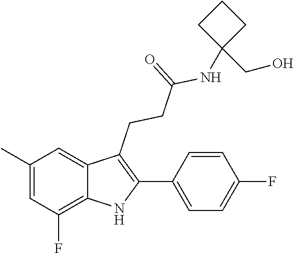 [SMILES: Cc1cc(F)c2[nH]c(-c3ccc(F)cc3)c(CCC(=O)NC3(CO)CCC3)c2c1] | 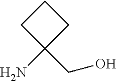 | LCMS m/z 399 [M + H]⁺ |
| 217 | 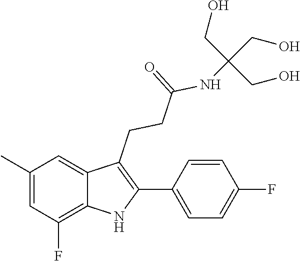 [SMILES: Cc1cc(F)c2[nH]c(-c3ccc(F)cc3)c(CCC(=O)NC(CO)(CO)CO)c2c1] | 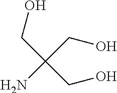 | LCMS m/z 419.13 [M + H]⁺ |
| 218 | 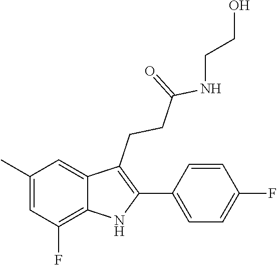 [SMILES: Cc1cc(F)c2[nH]c(-c3ccc(F)cc3)c(CCC(=O)NCCO)c2c1] | 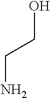 | LCMS m/z 359.16 [M + H]⁺ |
| 219 | 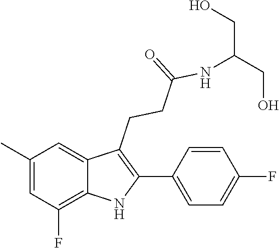 [SMILES: Cc1cc(F)c2[nH]c(-c3ccc(F)cc3)c(CCC(=O)NC(CO)CO)c2c1] | 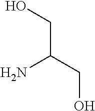 | LCMS m/z 389.16 [M + H]⁺ |
| 220 | 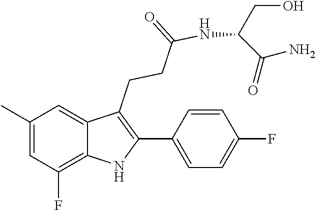 [SMILES: Cc1cc(F)c2[nH]c(-c3ccc(F)cc3)c(CCC(=O)N[C@H](CO)C(N)=O)c2c1] | 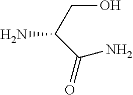 | LCMS m/z 401.9 [M + H]⁺ |
| 221 | 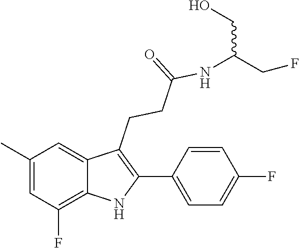 [SMILES: Cc1cc(F)c2[nH]c(-c3ccc(F)cc3)c(CCC(=O)NC(CO)CF)c2c1] | 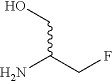 | LCMS m/z 391.02 [M + H]⁺ |
| 222 | 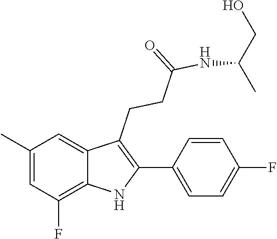 [SMILES: Cc1cc(F)c2[nH]c(-c3ccc(F)cc3)c(CCC(=O)N[C@@H](C)CO)c2c1] | 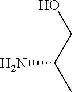 | LCMS m/z 373.17 [M + H]⁺ |
| 223 | 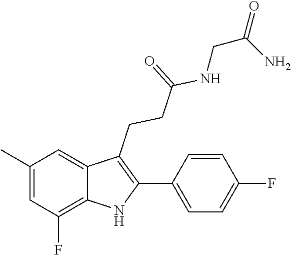 [SMILES: Cc1cc(F)c2[nH]c(-c3ccc(F)cc3)c(CCC(=O)NCC(N)=O)c2c1] | 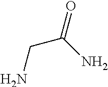 | LCMS m/z 371.99 [M + H]⁺ |
| 224 | 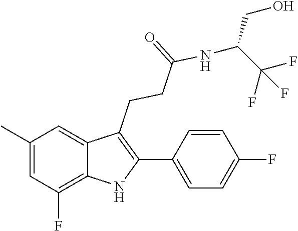 [SMILES: Cc1cc(F)c2[nH]c(-c3ccc(F)cc3)c(CCC(=O)N[C@H](CO)C(F)(F)F)c2c1] | 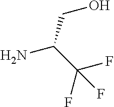 | LCMS m/z 427.11 [M + H]⁺ |
| 225 | 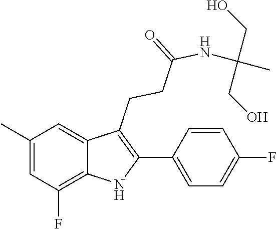 [SMILES: Cc1cc(F)c2[nH]c(-c3ccc(F)cc3)c(CCC(=O)NC(C)(CO)CO)c2c1] | 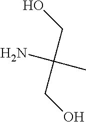 | LCMS m/z 403.07 [M + H]⁺ |
| 226 | 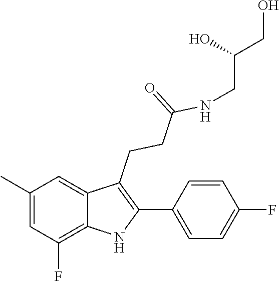 [SMILES: Cc1cc(F)c2[nH]c(-c3ccc(F)cc3)c(CCC(=O)NC[C@H](O)CO)c2c1] | 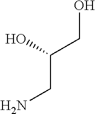 | LCMS m/z 389.06 [M + H]⁺ |

### Compound 227

### (R)-3-(5-bromo-7-fluoro-2-(4-fluorophenyl)-1H-indol-3-yl)-N-(1-hydroxypropan-2-yl)propenamide (227)

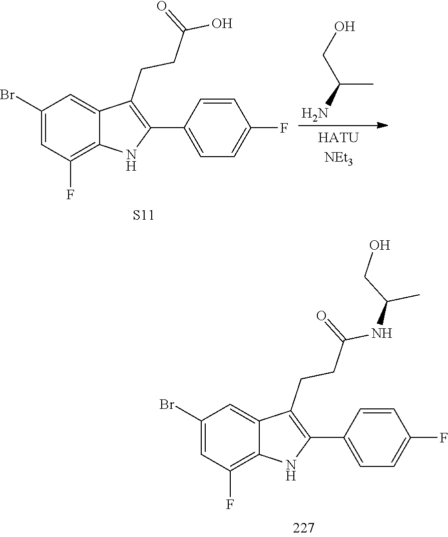

### Preparation of (R)-3-(5-bromo-7-fluoro-2-(4-fluorophenyl)-1H-indol-3-yl)-N-(1-hydroxypropan-2-yl)propenamide) (227)

To a solution of 3-[5-bromo-7-fluoro-2-(4-fluorophenyl)-1H-indol-3-yl]propanoic acid S11 (20.5 mg, 0.05367 mmol), (2R)-2-aminopropan-1-ol (6 mg, 0.07988 mmol) and HATU (41 mg, 0.1078 mmol) in DMSO (1 mL) was added TEA (50 μL, 0.3587 mmol). The reaction mixture was stirred at room temperature for 12 hours. Purification by reversed-phase HPLC (Method: C18 Waters Sunfire column (30×150 mm, 5 micron). Gradient: MeCN in H₂O with 0.1% trifluoroacetic acid) afforded 3-[5-bromo-7-fluoro-2-(4-fluorophenyl)-1H-indol-3-yl]-N-[(1R)-2-hydroxy-1-methyl-ethyl]propanamide (229) (16 mg, 68%). ¹H NMR (300 MHz, Methanol-d₄) δ 11.26 (s, 1H), 7.70-7.54 (m, 3H), 7.34-7.14 (m, 2H), 7.02 (dd, J=10.5, 1.6 Hz, 1H), 3.89 (dt, J=7.2, 5.9 Hz, 1H), 3.50-3.34 (m, 2H), 3.11 (dd, J=8.7, 6.8 Hz, 2H), 2.53-2.35 (m, 2H), 1.02 (d, J=6.8 Hz, 3H). LCMS m/z 437.05 [M+H]⁺.

### Compounds 228-229

Compounds 228-229 (see Table 6) were prepared in a single step from intermediate S11 using the method described for the preparation of compound 227. Amines were obtained from commercial sources.

TABLE 6
Structure and physicochemical data for compounds 228-229

| | | Amine | |
|---|---|---|---|
| Compound | Method/Product | Reagent | ¹H NMR; LCMS m/z [M + H]⁺ |
| 228 | As for Compound 227 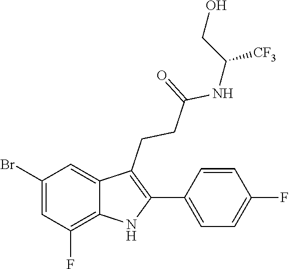 [SMILES: O=C(CCc1c(-c2ccc(F)cc2)[nH]c2c(F)cc(Br)cc12)N[C@H](CO)C(F)(F)F] | 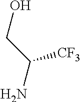 | ¹H NMR (300 MHz, Methanol- d₄) δ 11.27 (s, 1H), 8.46 (d, J = 9.3 Hz, 1H), 7.73-7.54 (m, 3H), 7.35-7.12 (m, 2H), 7.02 (dd, J = 10.5, 1.6 Hz, 1H), 4.62 (dddd, J = 9.3, 8.0, 6.4, 4.8 Hz, 1H), 3.75 (dd, J = 11.8, 4.8 Hz, 1H), 3.65 (dd, J = 11.8, 6.6 Hz, 1H), 3.22-2.97 (m, 2H), 2.77- 2.46 (m, 2H); LCMS m/z 491.02 [M + H]⁺ |
| 229 | As for Compound 227 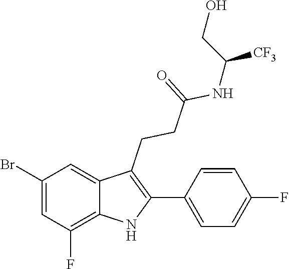 [SMILES: O=C(CCc1c(-c2ccc(F)cc2)[nH]c2c(F)cc(Br)cc12)N[C@@H](CO)C(F)(F)F] | 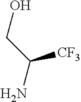 | ¹H NMR (300 MHz, Methanol- d₄) δ 7.70-7.50 (m, 3H), 7.33- 7.10 (m, 2H), 7.02 (dd, J = 10.5, 1.6 Hz, 1H), 4.71-4.47 (m, 1H), 3.76 (dd, J = 11.8, 4.8 Hz, 1H), 3.65 (dd, J = 11.8, 6.7 Hz, 1H), 3.22-3.02 (m, 2H), 2.77-2.52 (m, 2H); LCMS m/z 491.06 [M + H]⁺ |

### Compound 230

### 3-(7-cyclopropyl-2-phenyl-1H-indol-3-yl)-N-(5-oxo-1,2-dihydropyrrol-4-yl)propenamide (230)

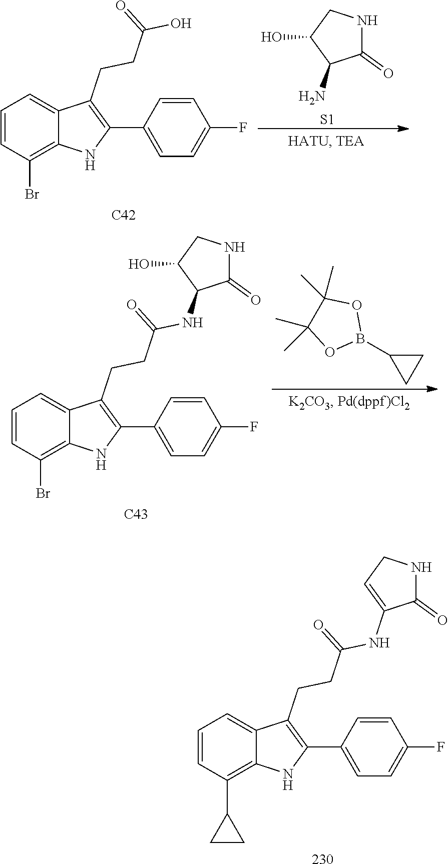

### Step 1. Synthesis of 3-(7-bromo-2-phenyl-1H-indol-3-yl)-N-[(3S,4R)-4-hydroxy-2-oxo-pyrrolidin-3-yl] propanamide (C43)

To a solution of 3-(7-bromo-2-phenyl-1H-indol-3-yl)propanoic acid C42 (413 mg, 1.160 mmol), (3S,4R)-3-amino-4-hydroxy-pyrrolidin-2-one S1 (138 mg, 1.188 mmol), and HATU (542 mg, 1.425 mmol) in DMSO (5 mL) was added TEA (500 μL, 3.587 mmol). The reaction was stirred at room temperature for an hour. Purification by reversed-phase chromatography (Column: C18. Gradient: 0-100% MeCN in water with 0.1% TFA) afforded the product. The desired peak fractions were evaporated in vacuo. Ethyl acetate (180 mL) was added. The organic layer was washed with brine, and NaHCO₃, dried over anhydrous sodium sulfate, filtered and concentrated in vacuo to afford 3-(7-bromo-2-phenyl-1H-indol-3-yl)-N-[(3S,4R)-4-hydroxy-2-oxo-pyrrolidin-3-yl] propanamide (C43) (421 mg, 81%). ¹H NMR (300 MHz, Chloroform-d) δ 8.23 (s, 1H), 7.65-7.51 (m, 5H), 7.45 (t, J=7.2 Hz, 1H), 7.38 (dd, J=7.6, 0.9 Hz, 1H), 7.06 (t, J=7.8 Hz, 1H), 6.21 (s, 1H), 5.55 (s, 1H), 5.52 (s, 1H), 4.22-4.16 (m, 1H), 4.00 (dd, J=8.1, 2.0 Hz, 1H), 3.67-3.55 (m, 1H), 3.37-3.15 (m, 3H), 2.70-2.57 (m, 2H). LCMS m/z 442.21 [M+H]⁺

### Step 2. Synthesis of 3-(7-cyclopropyl-2-phenyl-1H-indol-3-yl)-N-(5-oxo-1,2-dihydropyrrol-4-yl)propanamide (230)

To a solution of 3-(7-bromo-2-phenyl-1H-indol-3-yl)-N-[(3S,4R)-4-hydroxy-2-oxo-pyrrolidin-3-yl]propenamide C43 (26 mg, 0.05827 mmol) in 1,4-dioxane (2 mL) and water (0.5 mL) in a microwave tube was added 2-cyclopropyl-4,4,5,5-tetramethyl-1,3,2-dioxaborolane (20 mg, 0.1190 mmol), [1,1′-Bis(diphenylphosphino)ferrocene]dichloro-palladium(II) (8 mg, 0.009796 mmol), Cs₂CO₃ (60 mg, 0.1842 mmol), and 2-cyclopropyl-4,4,5,5-tetramethyl-1,3,2-dioxaborolane (20 mg, 0.1190 mmol). Reaction mixture was heated at 140° C. in the microwave for an hour. After cooling to room temperature, water (10 mL) was added. The aqueous layer was extracted with DCM. The organic layer was collected, and the solvent was removed in vacuo. Purification by reversed-phase HPLC (Method: C18 Waters Sunfire column (30×150 mm, 5 micron). Gradient: MeCN in H₂O with 0.1% trifluoroacetic acid) afforded the product (230) 3-(7-cyclopropyl-2-phenyl-1H-indol-3-yl)-N-(5-oxo-1,2-dihydropyrrol-4-yl)propanamide (2 mg, 9%). LCMS m/z 386.16 [M+H]⁺.

### Compound 231

### 3-[7-chloro-2-(4-fluorophenyl)-1H-indol-3-yl]-N-[(1S)-2,2-difluoro-1-(hydroxymethyl)ethyl]propanamide (231)

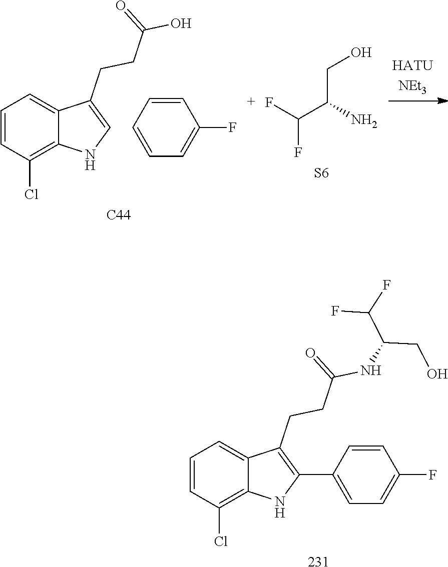

### Preparation of 3-[7-chloro-2-(4-fluorophenyl)-1H-indol-3-yl]-N-[(1S)-2,2-difluoro-1-(hydroxy-methyl)ethyl]propanamide (231)

To HATU (30 mg, 0.07890 mmol), (2S)-2-amino-3,3-difluoro-propan-1-ol (hydrochloride salt) S6 (8 mg, 0.04934 mmol) and 3-[7-chloro-2-(4-fluorophenyl)-1H-indol-3-yl]propanoic acid C44 (16 mg, 0.05036 mmol) in DMSO (0.5 mL) was added TEA (30 μL, 0.2152 mmol). The reaction was stirred at room temperature for 12 hours. The crude material was purified by reversed-phase HPLC (Method: C18 Waters Sunfire column (30×150 mm, 5 micron). Gradient: MeCN in H₂O with 0.1% trifluoroacetic acid) to afford 3-[7-chloro-2-(4-fluorophenyl)-1H-indol-3-yl]-N-[(1S)-2,2-difluoro-1-(hydroxymethyl)ethyl]propanamide (14.5 mg, 71%). LCMS m/z 411.08 [M+H]⁺

### Compound 232

### 3-[4,7-difluoro-2-(4-fluorophenyl)-1H-indol-3-yl]-N-[(1S)-2,2,2-trifluoro-1-(hydroxymethyl)ethyl]propenamide (232)

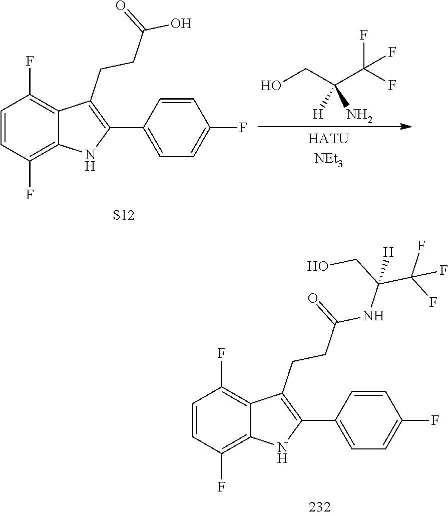 [SMILES: O=C(O)CCc1c(-c2ccc(F)cc2)[nH]c2c(F)ccc(F)c12 ; N[C@@H](CO)C(F)(F)F ; "S12" ; O=C(CCc1c(-c2ccc(F)cc2)[nH]c2c(F)ccc(F)c12)N[C@@H](CO)C(F)(F)F]

### Preparation of 3-[4,7-difluoro-2-(4-fluorophenyl)-1H-indol-3-yl]-N-[(1S)-2,2,2-trifluoro-1-(hydroxymethyl)ethyl]propenamide (232)

To 3-[4,7-difluoro-2-(4-fluorophenyl)-1H-indol-3-yl]propanoic acid S12 (12 mg, 0.03232 mmol), (2S)-2-amino-3,3,3-trifluoro-propan-1-ol (hydrochloride salt) (8 mg, 0.04833 mmol) and HATU (25 mg, 0.06575 mmol) in DMSO (1 mL) was added TEA (30 μL, 0.2152 mmol). The reaction was stirred at room temperature for 12 hours. Purification by reversed-phase HPLC (Method: C18 Waters Sunfire column (30×150 mm, 5 micron). Gradient: MeCN in H₂O with 0.1% trifluoroacetic acid) afforded 3-[4,7-difluoro-2-(4-fluorophenyl)-1H-indol-3-yl]-N-[(1S)-2,2,2-trifluoro-1-(hydroxymethyl)ethyl]propanamide (8.5 mg, 61%). LCMS m/z 431.09 [M+H]⁺.

### Compound 233

### 3-[4,7-difluoro-2-(4-fluorophenyl)-1H-indol-3-yl]-N-[(3S)-2-oxotetrahydrofuran-3-yl]propenamide (233)

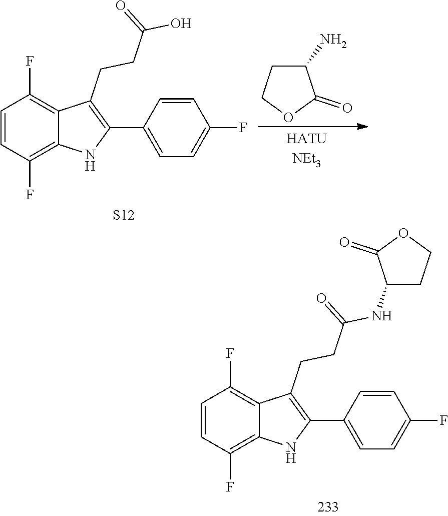

3-[4,7-difluoro-2-(4-fluorophenyl)-1H-indol-3-yl]-N-[(3 S)-2-oxotetrahydrofuran-3-yl]propenamide 233 was prepared in a single step from intermediate S12 using HATU as described for compound 232. Final product was isolated as 3-[4,7-difluoro-2-(4-fluorophenyl)-1H-indol-3-yl]-N-[(3S)-2-oxotetrahydrofuran-3-yl]propanamide (8.3 mg, 63%). LCMS m/z 402.97 [M+H]⁺.

### Compound 234

### 3-[2-(4-cyanophenyl)-5,7-difluoro-1H-indol-3-yl]-N-[(1S)-2,2,2-trifluoro-1-(hydroxymethyl)ethyl]propenamide (234)

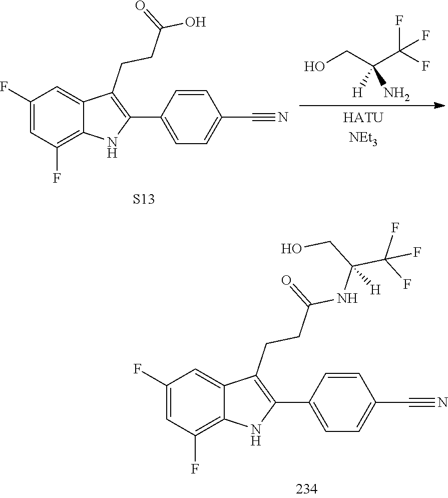 [SMILES: N#Cc1ccc(-c2[nH]c3c(F)cc(F)cc3c2CCC(=O)O)cc1 ; N[C@@H](CO)C(F)(F)F ; "S13" ; N#Cc1ccc(-c2[nH]c3c(F)cc(F)cc3c2CCC(=O)N[C@@H](CO)C(F)(F)F)cc1]

### Preparation of 3-[2-(4-cyanophenyl)-5,7-difluoro-1H-indol-3-yl]-N-[(1S)-2,2,2-trifluoro-1-(hydroxymethyl)ethyl]propenamide (234)

To a solution of 3-[2-(4-cyanophenyl)-5,7-difluoro-1H-indol-3-yl]propanoic acid S13 (14 mg, 0.04288 mmol), (2S)-2-amino-3,3,3-trifluoro-propan-1-ol (hydrochloride salt) (10.65 mg, 0.06433 mmol) and HATU (33 mg, 0.08679 mmol) in DMSO (1 mL) was added TEA (30 μL, 0.2152 mmol). The reaction was stirred at room temperature for 12 hours. The crude material was purified by reversed-phase HPLC (Method: C18 Waters Sunfire column (30×150 mm, 5 micron). Gradient: MeCN in H₂O with 0.1% trifluoroacetic acid) to afford 3-[2-(4-cyanophenyl)-5,7-difluoro-1H-indol-3-yl]-N-[(1S)-2,2,2-trifluoro-1-(hydroxymethyl)ethyl]propanamide (5.1 mg, 26%). ¹H NMR (300 MHz, Methanol-d₄) δ 7.92-7.82 (m, 4H), 7.19 (ddd, J=20.2, 9.3, 2.2 Hz, 1H), 6.79 (ddd, J=11.5, 9.6, 2.2 Hz, 1H), 4.60 (td, J=7.1, 4.9 Hz, 1H), 3.75 (dd, J=11.8, 4.8 Hz, 1H), 3.63 (dd, J=11.8, 6.6 Hz, 1H), 3.19 (td, J=8.0, 2.6 Hz, 2H), 2.74-2.49 (m, 2H). LCMS m/z 438.19 [M+H]⁺.

### Compound 235

### N-[(1S)-2,2-difluoro-1-(hydroxymethyl)ethyl]-3-(5-fluoro-2-phenyl-1H-indol-3-yl)propanamide (235)

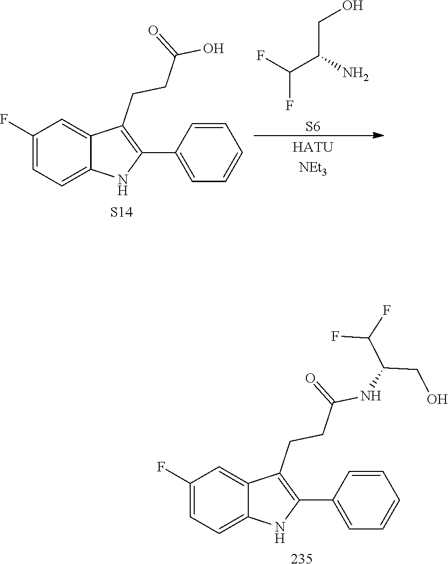

### Preparation of 3-[5-fluoro-2-(4-fluorophenyl)-1H-indol-3-yl]-N-[(1S)-2,2,2-trifluoro-1-(hydroxymethyl)ethyl]propanamide (235)

To a solution of (2S)-2-amino-3,3-difluoro-propan-1-ol hydrochloride S14 (8 mg, 0.05 mmol) in DMSO (0.5 mL) was added 3-(5-fluoro-2-phenyl-1H-indol-3-yl)propanoic acid S6 (16 mg, 0.056 mmol), HATU (16 mg, 0.056 mmol), and NEt₃ (30 μL, 0.22 mmol). The mixture was allowed to stir at room temperature for 12 hours. The mixture was then purified by reversed-phase HPLC (Method: C18 Waters Sunfire column (30×150 mm, 5 micron). Gradient: MeCN in H₂O with 0.1% trifluoroacetic acid) to afford the product (21 mg, 74%). LCMS m/z 377.1 [M+H]⁺.

### Compound 236

Compound 236 (see Table 7) was prepared from intermediate S14 using the appropriate reagent and using the amide formation method as described for compound 235.

TABLE 7
Structure and physicochemical data for compound 236

| | | | ¹H NMR; LCMS m/z |
|---|---|---|---|
| Compound | Product | Amine Reagent | [M + H]⁺ |
| 236 | 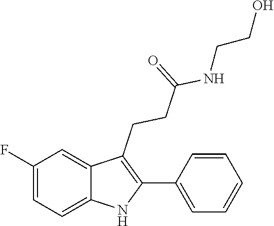 [SMILES: O=C(CCc1c(-c2ccccc2)[nH]c2ccc(F)cc12)NCCO] | 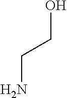 | ¹H NMR (400 MHz, Chloroform-d) δ 8.13 (s, 1H), 7.64-7.56 (m, 2H), 7.56-7.49 (m, 2H), 7.48- 7.36 (m, 1H), 7.38-7.29 (m, 2H), 6.98 (td, J = 9.0, 2.5 Hz, 1H), 5.75 (s, 1H), 3.58 (d, J = 5.2 Hz, 2H), 3.32 (td, J = 5.6, 4.4 Hz, 2H), 3.28-3.21 (m, 2H), 2.62-2.51 (m, 2H); LCMS m/z 327.21 [M + H]⁺ |

### Compounds 237-238

Compounds 237-238 (see Table 8) were obtained from commercial sources and may be prepared using analogous procedures to those used in the preparation of compounds 1-236.

TABLE 8
Structure and physicochemical data for compounds 237-238

| | | Amine | ¹H NMR; LCMS m/z |
|---|---|---|---|
| Compound | Product | Reagent | [M + H]⁺ |
| 237 | 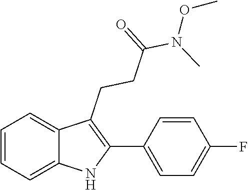 [SMILES: CON(C)C(=O)CCc1c(-c2ccc(F)cc2)[nH]c2ccccc12] | 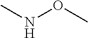 | LCMS m/z 327.21 [M + H]⁺ |
| 238 | 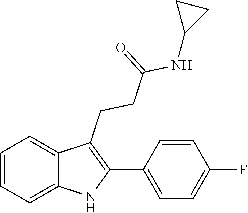 [SMILES: O=C(CCc1c(-c2ccc(F)cc2)[nH]c2ccccc12)NC1CC1] | 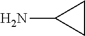 | LCMS m/z 323.24 [M + H]⁺ |

### Compound 239

### 4-(5,7-difluoro-2-(4-fluorophenyl)-1H-indol-3-yl)-N-methylbutanamide (239)

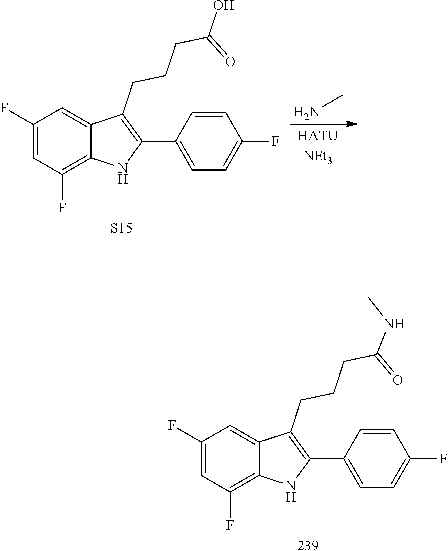

### Preparation of 3-[5-fluoro-2-(4-fluorophenyl)-1H-indol-3-yl]-N-[(1S)-2,2,2-trifluoro-1-(hydroxymethyl)ethyl]propanamide (239)

To a solution of 4-[5,7-difluoro-2-(4-fluorophenyl)-1H-indol-3-yl]butanoic acid S15 (16 mg, 0.097 mmol) in DMF (1 mL) was added methanamine (2 mg, 0.65 mmol), HATU (30 mg, 0.07 mmol), and NEt₃ (18 μL, 0.13 mmol). The mixture was allowed to stir at room temperature for 2 hours. The mixture was then purified by reversed-phase HPLC (Method: C18 Waters Sunfire column (30×150 mm, 5 micron). Gradient: MeCN in H₂O with 0.1% trifluoroacetic acid) to afford the product (21 mg, 74%). ¹H NMR (300 MHz, Chloroform-d) δ 7.73-7.46 (m, 2H), 7.31-7.15 (m, 2H), 7.10 (dd, J=9.4, 2.2 Hz, 1H), 6.72 (ddd, J=11.0, 9.6, 2.2 Hz, 1H), 2.93-2.73 (m, 2H), 2.67 (s, 3H), 2.20 (t, J=7.3 Hz, 2H), 1.94 (p, J=7.5 Hz, 2H).

### Compounds 240-256

Compounds 240-256 (see Table 9) were prepared from intermediate S15 using the appropriate reagent and using the amide formation method as described for compound 239. Amines were prepared by methods described above or obtained from commercial sources.

TABLE 9
Structure and physicochemical data for compound 240-256

| | | Amine | ¹H NMR; LCMS m/z |
|---|---|---|---|
| Compound | Product | Reagent | [M + H]₊ |
| 240 | 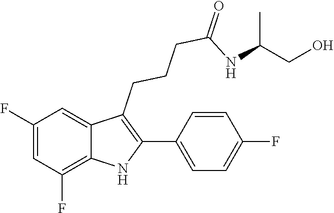 [SMILES: C[C@@H](CO)NC(=O)CCCc1c(-c2ccc(F)cc2)[nH]c2c(F)cc(F)cc12] | 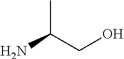 | ¹H NMR (300 MHz, Chloroform-d) δ 8.23 (s, 1H), 7.64-7.44 (m, 2H), 7.24-7.12 (m, 2H), 7.09 (dd, J = 9.1, 2.2 Hz, 1H), 6.76 (ddd, J = 10.8, 9.4, 2.2 Hz, 1H), 5.51 (d, J = 7.1 Hz, 1H), 4.06 (ddt, J = 10.3, 6.8, 3.4 Hz, 1H), 3.66 (dd, J = 10.9, 3.5 Hz, 1H), 3.51 (dd, J = 11.0, 6.2 Hz, 1H), 2.83 (dd, J = 8.6, 6.7 Hz, 2H), 2.21 (t, |
| | | | J = 7.3 Hz, 2H), 2.09-1.91 |
| | | | (m, 2H), 1.14 (d, J = 6.8 |
| | | | Hz, 3H); LCMS m/z |
| | | | 391.36 [M + H]⁺ |
| 241 | 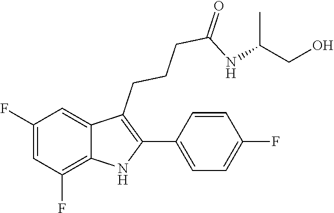 [SMILES: C[C@H](CO)NC(=O)CCCc1c(-c2ccc(F)cc2)[nH]c2c(F)cc(F)cc12] | 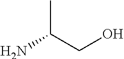 | ¹H NMR (300 MHz, Chloroform-d) δ 8.21 (s, 1H), 7.66-7.43 (m, 2H), 7.25-7.13 (m, 2H), 7.10 (dd, J = 9.1, 2.2 Hz, 1H), 6.76 (ddd, J = 11.4, 9.5, 2.2 Hz, 1H), 5.50 (d, J = 7.2 Hz, 1H), 4.06 (dtq, J = 10.3, 6.8, 3.3 Hz, 1H), 3.66 (dd, J = 10.9, 3.4 Hz, 1H), 3.51 (dd, J = 11.0, 6.2 Hz, 1H), 2.92-2.82 (m, 2H), 2.22 (t, J = 7.3 |
| | | | Hz, 2H), 2.00 (p, J = 7.4 |
| | | | Hz, 2H), 1.14 (d, J = 6.8 |
| | | | Hz, 3H); LCMS m/z |
| | | | 391.32 [M + H]⁺ |
| 242 | 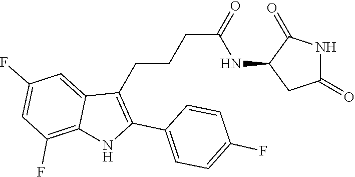 [SMILES: O=C1C[C@@H](NC(=O)CCCc2c(-c3ccc(F)cc3)[nH]c3c(F)cc(F)cc23)C(=O)N1] | 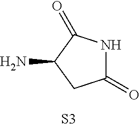 | LCMS m/z 430.11 [M + H]⁺ |
| 243 | 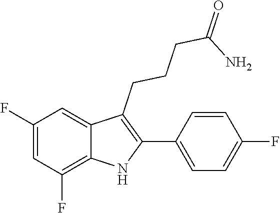 [SMILES: NC(=O)CCCc1c(-c2ccc(F)cc2)[nH]c2c(F)cc(F)cc12] | NH₃ | ¹H NMR (300 MHz, Chloroform-d) δ 7.63 (dd, J = 8.7, 5.3 Hz, 2H), 7.22 (d, J = 8.8 Hz, 2H), 7.12 (dd, J = 9.3, 2.2 Hz, 1H), 6.80-6.66 (m, 1H), 2.96- 2.65 (m, 2H), 2.25 (t, J = 7.3 Hz, 2H), 1.95 (p, J = 7.5 Hz, 2H); LCMS m/z 333.1 [M + H]⁺ |
| 244 | 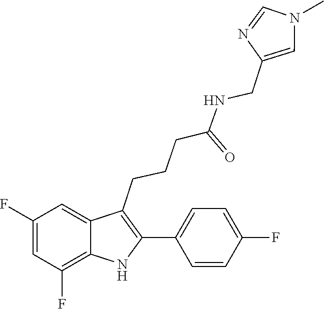 [SMILES: Cn1cnc(CNC(=O)CCCc2c(-c3ccc(F)cc3)[nH]c3c(F)cc(F)cc23)c1] | 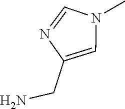 | ¹H NMR (300 MHz, Chloroform-d) δ 8.84- 8.62 (m, 1H), 7.72-7.52 (m, 2H), 7.39 (d, J = 1.5 Hz, 1H), 7.32-7.15 (m, 2H), 7.07 (dd, J = 9.4, 2.2 Hz, 1H), 6.73 (ddd, J = 11.0, 9.6, 2.2 Hz, 1H), 4.35 (d, J = 0.9 Hz, 2H), 3.87 (s, 3H), 2.98-2.69 (m, 2H), 2.27 (t, J = 7.4 Hz, 2H), 1.96 (p, J = 7.5 Hz, 2H); LCMS m/z 427.14 [M + H]⁺ |
| 245 | 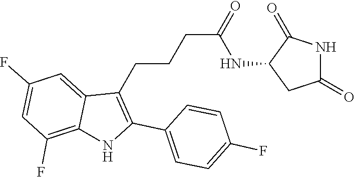 [SMILES: O=C1C[C@H](NC(=O)CCCc2c(-c3ccc(F)cc3)[nH]c3c(F)cc(F)cc23)C(=O)N1] | 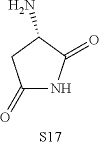 | LCMS m/z 430.34 [M + H]⁺ |
| 246 | 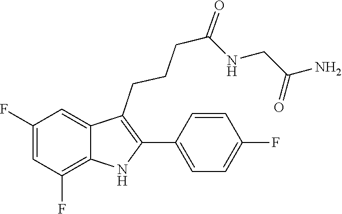 [SMILES: NC(=O)CNC(=O)CCCc1c(-c2ccc(F)cc2)[nH]c2c(F)cc(F)cc12] | 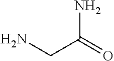 | LCMS m/z 390.11 [M + H]⁺ |
| 247 | 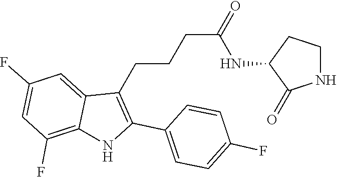 [SMILES: O=C(CCCc1c(-c2ccc(F)cc2)[nH]c2c(F)cc(F)cc12)N[C@@H]1CCNC1=O] | 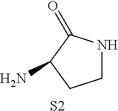 | LCMS m/z 416.38 [M + H]⁺ |
| 248 | 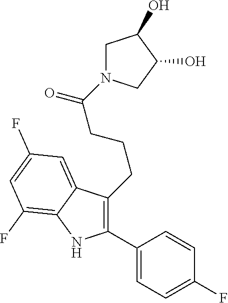 [SMILES: O=C(CCCc1c(-c2ccc(F)cc2)[nH]c2c(F)cc(F)cc12)N1C[C@@H](O)[C@H](O)C1] | 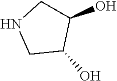 | LCMS m/z 419.16 [M + H]⁺ |
| 249 | 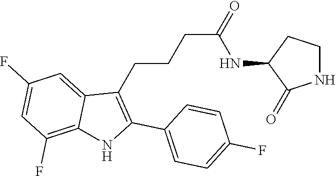 [SMILES: O=C(CCCc1c(-c2ccc(F)cc2)[nH]c2c(F)cc(F)cc12)N[C@H]1CCNC1=O] | 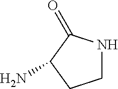 | ¹H NMR (300 MHz, Chloroform-d) δ 8.29 (s, 1H), 7.61-7.41 (m, 2H), 7.24-7.12 (m, 2H), 7.09 (dd, J = 9.1, 2.2 Hz, 1H), 6.74 (ddd, J = 10.8, 9.4, 2.2 Hz, 1H), 6.19-5.86 (m, 2H), 4.30 (ddd, J = 10.9, 8.2, 5.3 Hz, 1H), 3.37 (ddd, J = 9.8, 3.5, 1.3 Hz, 2H), 2.90-2.68 |
| | | | (m, 3H), 2.24 (td, J = 7.2, |
| | | | 2.4 Hz, 2H), 1.99 (p, J = |
| | | | 7.3 Hz, 2H), 1.82 (ddt, J = |
| | | | 12.6, 11.0, 9.6 Hz, 1H); |
| | | | LCMS m/z 416.34 |
| | | | [M + H]⁺ |
| 250 | 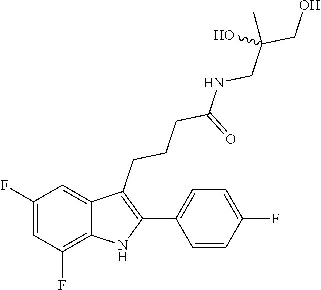 [SMILES: CC(O)(CO)CNC(=O)CCCc1c(-c2ccc(F)cc2)[nH]c2c(F)cc(F)cc12] | 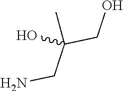 | LCMS m/z 421.15 [M + H]⁺ |
| 251 | 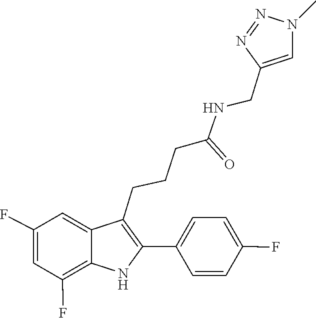 [SMILES: Cn1cc(CNC(=O)CCCc2c(-c3ccc(F)cc3)[nH]c3c(F)cc(F)cc23)nn1] | 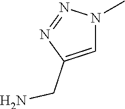 | LCMS m/z 428.15 [M + H]⁺ |
| 252 | 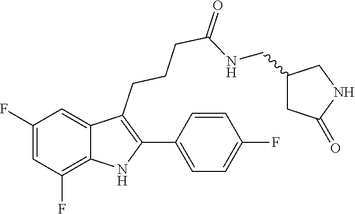 [SMILES: O=C(CCCc1c(-c2ccc(F)cc2)[nH]c2c(F)cc(F)cc12)NCC1CNC(=O)C1] | 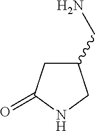 | LCMS m/z 430.14 [M + H]⁺ |
| 253 | 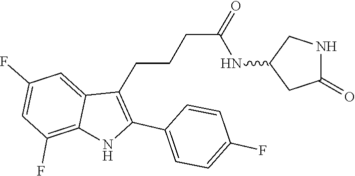 [SMILES: O=C1CC(NC(=O)CCCc2c(-c3ccc(F)cc3)[nH]c3c(F)cc(F)cc23)CN1] | 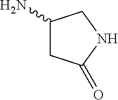 | LCMS m/z 416.23 [M + H]⁺ |
| 254 | 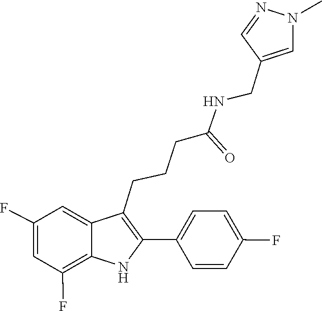 [SMILES: Cn1cc(CNC(=O)CCCc2c(-c3ccc(F)cc3)[nH]c3c(F)cc(F)cc23)cn1] | 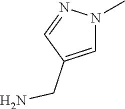 | LCMS m/z 426.88 [M + H]⁺ |
| 255 | 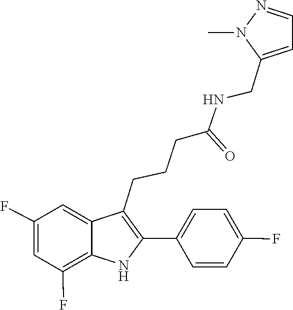 [SMILES: Cn1nccc1CNC(=O)CCCc1c(-c2ccc(F)cc2)[nH]c2c(F)cc(F)cc12] | 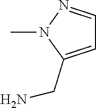 | LCMS m/z 427.18 [M + H]⁺ |
| 256 | 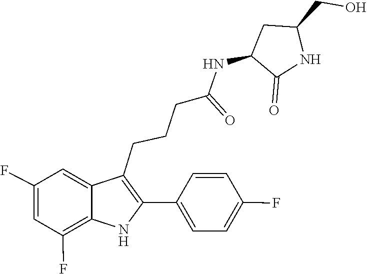 [SMILES: O=C(CCCc1c(-c2ccc(F)cc2)[nH]c2c(F)cc(F)cc12)N[C@H]1C[C@@H](CO)NC1=O] | 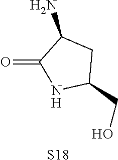 | LCMS m/z 445.97 [M + H]⁺ |

### Compound 257

### (5S)-3-[2-[5,7-difluoro-2-(4-fluorophenyl)-1H-indol-3-yl]ethyl]-5-methyl-imidazolidine-2,4-dione (257)

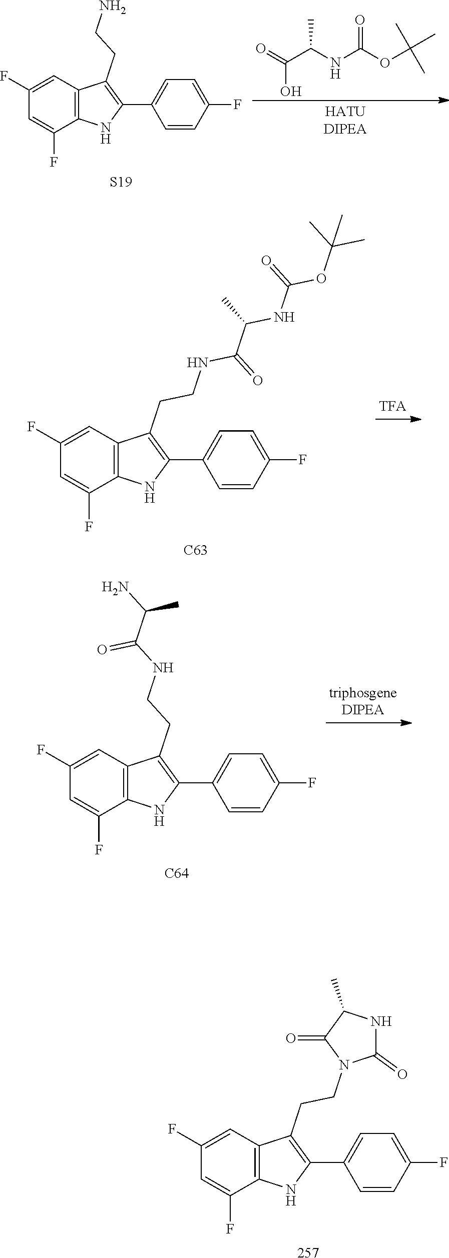

### Step 1. Synthesis of tert-butyl N-[(1S)-2-[2-[5,7-difluoro-2-(4-fluorophenyl)-1H-indol-3-yl]ethylamino]-1-methyl-2-oxo-ethyl]carbamate (C63)

To a solution of 2-[5,7-difluoro-2-(4-fluorophenyl)-1H-indol-3-yl]ethanamine (75 mg, 0.2282 mmol) S19 and DIPEA (100 μL, 0.575 mmol) in DMF (3 mL) was added (2S)-2-(tert-butoxycarbonylamino)propanoic acid (55 mg, 0.2907 mmol and HATU (100 mg, 0.2630 mmol). The mixture was stirred overnight and purified by reversed-phase chromatography (Column: C18. Gradient: 0-100% MeCN in water with 0.1% trifluoroacetic acid) to yield tert-butyl N-[(1S)-2-[2-[5,7-difluoro-2-(4-fluorophenyl)-1H-indol-3-yl]ethylamino]-1-methyl-2-oxo-ethyl]carbamate (55 mg, 46%). LCMS m/z 462.18 [M+H]⁺.

### Step 2. Synthesis of (2S)-2-amino-N-[2-[5,7-difluoro-2-(4-fluorophenyl)-1H-indol-3-yl]ethyl]propenamide (C64)

To solution of tert-butyl N-[(1S)-2-[2-[5,7-difluoro-2-(4-fluorophenyl)-1H-indol-3-yl]ethylamino]-1-methyl-2-oxo-ethyl]carbamate (25 mg, 0.05417 mmol) C63 in DCM (10 mL) was added TFA (100 μL, 1.298 mmol) the reaction was stirred overnight. The reaction was then concentrated and re-dissolved in DMSO (5 mL). The crude residue was then purified by reversed-phase HPLC (Method: C18 Waters Sunfire column (30×150 mm, 5 micron). Gradient: MeCN in H₂O with 0.1% trifluoroacetic acid) to yield (2S)-2-amino-N-[2-[5,7-difluoro-2-(4-fluorophenyl)-1H-indol-3-yl]ethyl]propanamide (5.5 mg, 25%). ¹H NMR (300 MHz, Acetone-d₆) δ 7.84-7.73 (m, 2H), 7.35-7.23 (m, 3H), 6.85 (ddd, J=11.3, 9.6, 2.2 Hz, 1H), 4.90 (q, J=7.0 Hz, 1H), 3.58-3.47 (m, 2H), 3.30 (d, J=0.8 Hz, 1H), 3.11-3.00 (m, 2H), 2.75-2.47 (m, 1H), 1.57 (dd, J=10.9, 7.0 Hz, 3H). LCMS m/z 362.14 [M+H]⁺.

### Step 3. Synthesis of (5S)-3-[2-[5,7-difluoro-2-(4-fluorophenyl)-1H-indol-3-yl]ethyl]-5-methyl-imidazolidine-2,4-dione (257)

To a solution of (2S)-2-amino-N-[2-[5,7-difluoro-2-(4-fluorophenyl)-1H-indol-3-yl]ethyl]propenamide C64 (25 mg, 0.06581 mmol) dissolved in DCM (5 mL) was added triphosgene (20 mg, 0.0674 mmol) and DIPEA (50 μL, 0.287 mmol). The reaction was stirred for 3 hours at room temperature at which point it was concentrated and re-dissolved in DMSO (5 mL). This crude solution was purified by reversed-phase HPLC (Method: C18 Waters Sunfire column (30×150 mm, 5 micron). Gradient: MeCN in H₂O with 0.1% trifluoroacetic acid) to yield (5 S)-3-[2-[5,7-difluoro-2-(4-fluorophenyl)-1H-indol-3-yl]ethyl]-5-methyl-imidazolidine-2,4-dione (17.5 mg, 59%). ¹H NMR (300 MHz, Acetone-d₆) δ 7.99-7.67 (m, 3H), 7.44-7.21 (m, 3H), 7.14 (s, 1H), 6.84 (ddd, J=11.0, 9.7, 2.2 Hz, 1H), 4.01 (qd, J=7.0, 1.2 Hz, 1H), 3.91 (ddt, J=13.3, 6.7, 3.4 Hz, 1H), 3.86-3.60 (m, 2H), 3.39 (qd, J=7.4, 4.4 Hz, 2H), 3.21-2.93 (m, 2H), 1.25 (d, J=7.0 Hz, 3H). LCMS m/z 388.1 [M+H]⁺.

### Compound 258

### 3-[2-[5,7-difluoro-2-(4-fluorophenyl)-1H-indol-3-yl]ethyl]imidazolidine-2,4-dione (258)

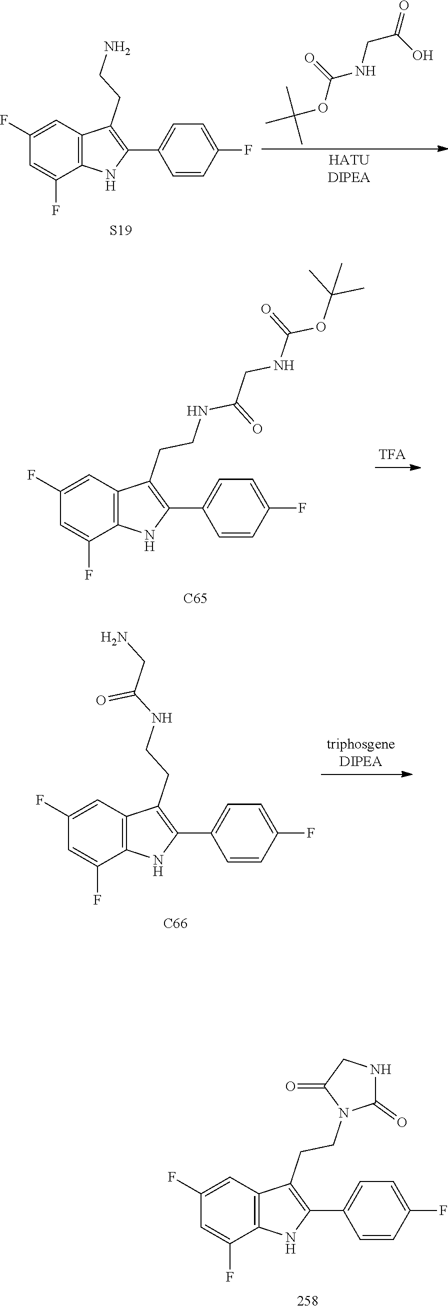

### Step 1. Synthesis of tert-butyl N-[(1S)-2-[2-[5,7-difluoro-2-(4-fluorophenyl)-1H-indol-3-yl]ethylamino]-1-methyl-2-oxo-ethyl]carbamate (C65)

To a solution of 2-[5,7-difluoro-2-(4-fluorophenyl)-1H-indol-3-yl]ethanamine (75 mg, 0.2282 mmol) S19 and DIPEA (100 μL, 0.575 mmol) in DMF (3 mL) was added 2-(tert-butoxycarbonylamino)acetic acid (50 mg, 0.2854 mmol) and HATU (100 mg, 0.2630 mmol). The mixture was stirred overnight and purified by reversed-phase chromatography (Column: C18. Gradient: 0-100% MeCN in water with 0.1% trifluoroacetic acid) to yield tert-butyl N-[2-[2-[5,7-difluoro-2-(4-fluorophenyl)-1H-indol-3-yl]ethylamino]-2-oxo-ethyl]carbamate (65 mg, 35%). LCMS m/z 448.23 [M+H]⁺.

### Step 2. Synthesis of 2-amino-N-[2-[5,7-difluoro-2-(4-fluorophenyl)-1H-indol-3-yl]ethyl]acetamide (C66)

To a solution of tert-butyl N-[(1S)-2-[2-[5,7-difluoro-2-(4-fluorophenyl)-1H-indol-3-yl]ethylamino]-1-methyl-2-oxo-ethyl]carbamate C65 (25 mg, 0.04930 mmol) in DCM (10 mL) was added TFA (100 μL, 1.298 mmol) and reaction was stirred overnight. The reaction was then concentrated, dissolved in DMSO (1 mL), and purified by reversed-phase HPLC (Method: C18 Waters Sunfire column (30×150 mm, 5 micron). Gradient: MeCN in H₂O with 0.1% trifluoroacetic acid) to yield 2-amino-N-[2-[5,7-difluoro-2-(4-fluorophenyl)-1H-indol-3-yl]ethyl]acetamide (Trifluoroacetate salt) (5.3 mg, 21%). ¹H NMR (300 MHz, Acetone-d₆) δ 7.83-7.72 (m, 2H), 7.35-7.23 (m, 3H), 6.84 (ddd, J=11.1, 9.7, 2.2 Hz, 1H), 4.58 (s, 2H), 3.57 (dt, J=15.4, 7.7 Hz, 2H), 3.06 (dd, J=9.1, 6.4 Hz, 2H). LCMS m/z 348.14 [M+H]⁺.

### Step 3. Synthesis of 3-[2-[5,7-difluoro-2-(4-fluorophenyl)-1H-indol-3-yl]ethyl]imidazolidine-2,4-dione (258)

To a solution of 2-amino-N-[2-[5,7-difluoro-2-(4-fluorophenyl)-1H-indol-3-yl]ethyl]acetamide C66 (25 mg, 0.0654 mmol) dissolved in DCM (5 mL) was added triphosgene (20 mg, 0.0674 mmol) and DIPEA (50 μL, 0.287 mmol) and the reaction was stirred overnight. The reaction was then concentrated, re-dissolved in DMSO (5 mL), and purified by reversed-phase HPLC (Method: C18 Waters Sunfire column (30×150 mm, 5 micron). Gradient: 0-100% MeCN in H₂O with 0.1% trifluoroacetic acid) to yield 3-[2-[5,7-difluoro-2-(4-fluorophenyl)-1H-indol-3-yl]ethyl]imidazolidine-2,4-dione (15.9 mg, 58%). ¹H NMR (300 MHz, Acetone-d₆) δ 8.03-7.63 (m, 2H), 7.49-7.23 (m, 3H), 7.05 (s, 1H), 6.84 (ddd, J=11.1, 9.7, 2.2 Hz, 1H), 3.85 (d, J=1.0 Hz, 2H), 3.79-3.68 (m, 2H), 3.20-2.92 (m, 2H). LCMS m/z 374.02 [M+H]⁺.

### Compound 259

### 1-[2-[5,7-difluoro-2-(4-fluorophenyl)-1H-indol-3-yl]ethyl]-3-[1-(fluoromethyl)-2-hydroxy-ethyl]urea (259)

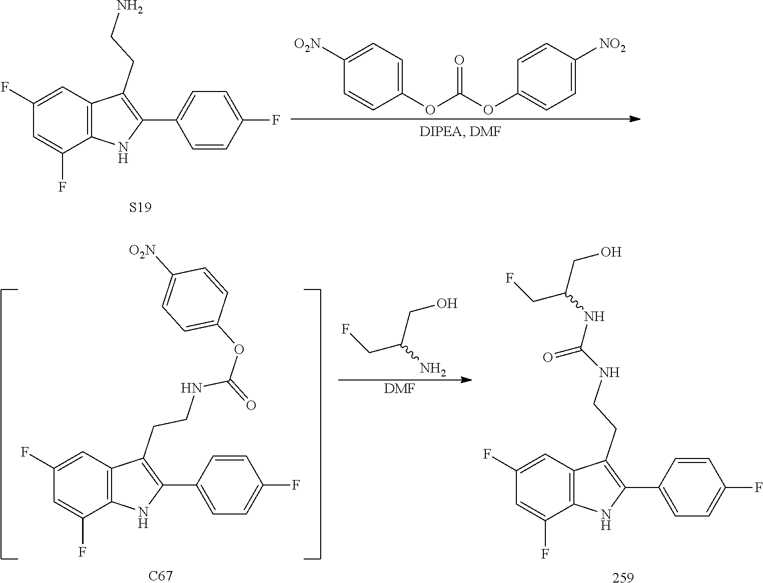

### Preparation of 1-[2-[5,7-difluoro-2-(4-fluorophenyl)-1H-indol-3-yl]ethyl]-3-[1-(fluoromethyl)-2-hydroxy-ethyl]urea (259)

To a solution of 2-[5,7-difluoro-2-(4-fluorophenyl)-1H-indol-3-yl]ethanamine as a TFA salt S19 (297 mg, 0.73 mmol) in DMF (5 mL) was added a solution of bis(4-nitrophenyl) carbonate (268 mg, 0.88 mmol) in DMF (5 mL) and DIPEA (300 μL, 1.72 mmol). The reaction was stirred for 1 hour and then 2-amino-3-fluoro-propan-1-ol (HCl salt) (120 mg, 0.93 mmol) was added and the reaction was stirred overnight. The reaction was purified by reverse-phase chromatography (C18 column; Gradient: 0-100% MeCN in H₂O with 0.1% TFA) to afford the title compound (280 mg, 85%). ¹H NMR (400 MHz, Acetone-d₆) δ 7.97-7.73 (m, 2H), 7.36-7.25 (m, 2H), 6.92-6.72 (m, 1H), 4.58 (dd, J=8.9, 4.5 Hz, 1H), 4.47 (ddd, J=8.9, 7.6, 5.0 Hz, 1H), 4.36 (dd, J=8.9, 5.5 Hz, 1H), 3.71-3.64 (m, 1H), 3.64-3.54 (m, 2H), 3.54-3.40 (m, 3H), 3.19-2.85 (m, 2H). LCMS m/z 410.2 [M+H]⁺.

### Compounds 260-303

Compounds 260-303 (see Table 10) were prepared in a single step from intermediate S19 using the appropriate reagents and the amide coupling method as described for the preparation of compound 259. Amines were prepared by methods described above or obtained from commercial sources. Any modifications to methods are noted in Table 10 and accompanying footnotes.

TABLE 10
Method of preparation, structure, physicochemical data for compounds 260-303

| | | Amine | |
|---|---|---|---|
| Compound | Method/Product | Reagent | ¹H NMR; LCMS m/z [M + H]⁺ |
| 260 | As for Compound 259 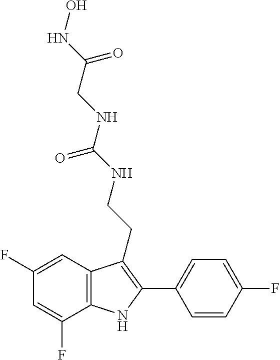 [SMILES: O=C(CNC(=O)NCCc1c(-c2ccc(F)cc2)[nH]c2c(F)cc(F)cc12)NO] | 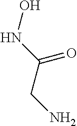 | ¹H NMR (300 MHz, Acetone- d₆) δ 10.70 (s, 1H), 7.99-7.74 (m, 2H), 7.53-7.05 (m, 3H), 6.82 (ddd, J = 11.1, 9.7, 2.2 Hz, 1H), 6.10 (s, 1H), 5.90 (s, 1H), 3.74 (d, J = 9.5 Hz, 2H), 3.45 (d, J = 8.0 Hz, 2H), 3.19- 2.88 (m, 4H); LCMS m/z 406.89 [M + H]⁺ |
| 261 | As for Compound 259 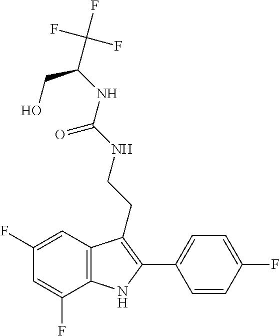 [SMILES: O=C(NCCc1c(-c2ccc(F)cc2)[nH]c2c(F)cc(F)cc12)N[C@@H](CO)C(F)(F)F] | 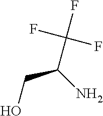 | ¹H NMR (300 MHz, Acetone- d₆) δ 10.70 (s, 1H), 7.93-7.66 (m, 2H), 7.44-7.11 (m, 3H), 6.83 (ddd, J = 11.1, 9.6, 2.2 Hz, 1H), 6.08 (d, J = 9.7 Hz, 1H), 4.58 (ddp, J = 13.1, 8.6, 4.3 Hz, 1H), 4.03-3.69 (m, 3H), 3.55-3.45 (m, 3H), 3.18- 2.88 (m, 2H); LCMS m/z 446.16 [M + H]⁺ |
| 262 | As for Compound 259 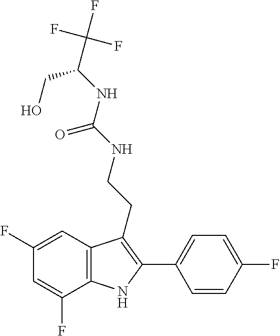 [SMILES: O=C(NCCc1c(-c2ccc(F)cc2)[nH]c2c(F)cc(F)cc12)N[C@H](CO)C(F)(F)F] | 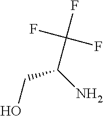 | ¹H NMR (300 MHz, Acetone-d₆) δ 10.70 (s, 1H), 7.91-7.72 (m, 2H), 7.47-7.19 (m, 3H), 6.83 (ddd, J = 11.2, 9.7, 2.2 Hz, 1H), 6.09 (d, J = 9.3 Hz, 2H), 4.76-4.45 (m, 1H), 3.99-3.69 (m, 3H), 3.35 (s, 1H), 3.17- 2.91 (m, 3H); LCMS m/z 446.2 [M + H]⁺ |
| 263 | As for Compound 259 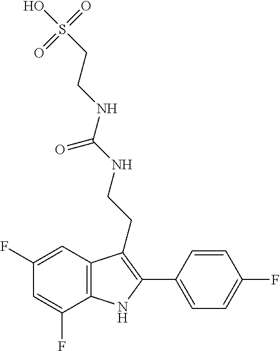 [SMILES: O=C(NCCc1c(-c2ccc(F)cc2)[nH]c2c(F)cc(F)cc12)NCCS(=O)(=O)O] | 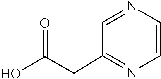 | ¹H NMR (300 MHz, Acetone- d₆) δ 7.77 (dd, J = 8.5, 5.4 Hz, 2H), 7.29 (t, J = 10.4 Hz, 2H), 6.82 4, J = 10.1 Hz, 1H), 3.65 (t, J = 5.5 Hz, 2H), 3.52 (t, J = 7.7 Hz, 2H), 3.15-2.99 (m, 3H), 3.01-2.71 (m, 2H); LCMS m/z 441.81 [M + H]⁺ |
| 264 | As for Compound 259 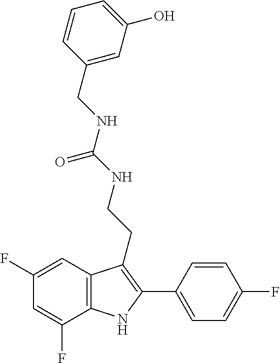 [SMILES: O=C(NCCc1c(-c2ccc(F)cc2)[nH]c2c(F)cc(F)cc12)NCc1cccc(O)c1] | 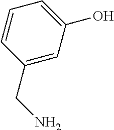 | ¹H NMR (300 MHz, Acetone- d₆) δ 8.00-7.54 (m, 2H), 7.34 (dd, J =9.5, 2.2 Hz, 1H), 7.29- 7.09 (m, 3H), 7.00-6.54 (m, 4H), 5.82 (d, J = 28.3 Hz, 2H), 4.29 (d, J = 5.2 Hz, 2H), 3.48 (d, J = 8.5 Hz, 2H), 3.15-2.87 (m, 4H); LCMS m/z 440.27 [M + H]⁺ |
| 265 | As for Compound 259 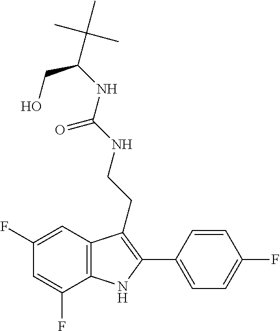 [SMILES: CC(C)(C)[C@H](CO)NC(=O)NCCc1c(-c2ccc(F)cc2)[nH]c2c(F)cc(F)cc12] | 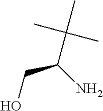 | ¹H NMR (300 MHz, Acetone- d₆) δ 10.70 (s, 1H), 8.09-7.66 (m, 2H), 7.51-7.06 (m, 3H), 6.82 (ddd, J = 11.1, 9.7, 2.2 Hz, 1H), 5.60 (s, 1H), 4.30 (s, 1H), 4.10 (s, 1H), 3.73 (dd, J = 10.5, 3.2 Hz, 1H), 3.50 (ddd, J = 9.9, 4.9, 1.9 Hz, 4H), 3.02 (d, J = 6.7 Hz, 2H), 0.93 (s, 9H); LCMS m/z 434.33 [M + H]⁺ |
| 266 | As for Compound 259 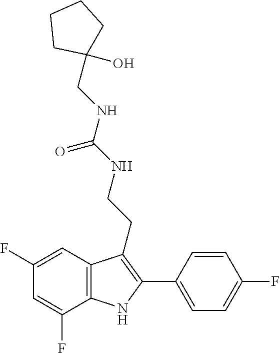 [SMILES: O=C(NCCc1c(-c2ccc(F)cc2)[nH]c2c(F)cc(F)cc12)NCC1(O)CCCC1] | 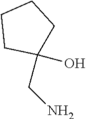 | ¹H NMR (300 MHz, Acetone- d₆) δ 7.94-7.68 (m, 2H), 7.44- 7.12 (m, 3H), 6.82 (ddd, J = 11.1, 9.7, 2.2 Hz, 1H), 3.48 (dd, J = 8.7, 6.4 Hz, 4H), 3.31 (s, 2H), 3.12-2.84 (m, 2H), 1.93-1.67 (m, 2H), 1.67-1.34 (m, 6H); LCMS m/z 432.28 [M + H]⁺ |
| 267 | As for Compound 259 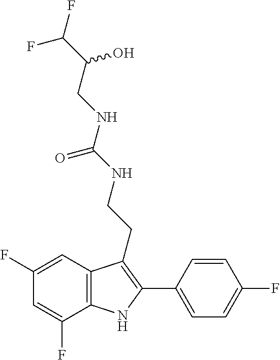 [SMILES: O=C(NCCc1c(-c2ccc(F)cc2)[nH]c2c(F)cc(F)cc12)NCC(O)C(F)F] | 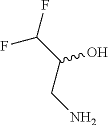 | ¹H NMR (300 MHz, Acetone- d₆) δ 8.08-7.44 (m, 2H), 7.41- 7.15 (m, 3H), 6.83 (ddd, J = 11.1, 9.7, 2.2 Hz, 1H), 5.76 (td, J = 55.8, 4.5 Hz, 2H), 3.89- 3.69 (m, 1H), 3.45 (d, J = 14.9 Hz, 3H), 3.36-3.22 (m, 1H), 3.21-2.91 (m, 3H); LCMS m/z 427.8 [M + H]⁺ |
| 268 | As for Compound 259 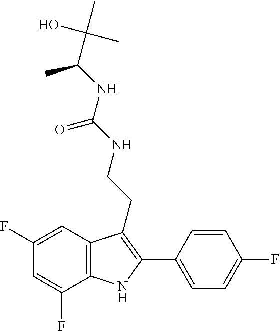 [SMILES: C[C@H](NC(=O)NCCc1c(-c2ccc(F)cc2)[nH]c2c(F)cc(F)cc12)C(C)(C)O] | 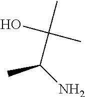 | ¹H NMR (300 MHz, Acetone- d₆) δ 10.75 (s, 1H), 8.00-7.61 (m, 2H), 7.29 (dtd, J = 8.9, 6.7, 2.2 Hz, 3H), 6.82 (ddd, J = 11.0, 9.6, 2.2 Hz, 1H), 5.50 (s, 1H), 4.50 (s, 2H), 3.68 (q, J = 6.7 Hz, 1H), 3.48 (dd, J = 8.5, 6.6 Hz, 2H), 3.22-2.84 (m, 2H), 1.10 (dd, J = 12.3, 5.8 Hz, 9H); LCMS m/z 419.82 [M + H]⁺ |
| 269 | As for Compound 259 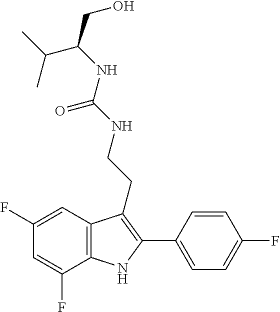 [SMILES: CC(C)[C@@H](CO)NC(=O)NCCc1c(-c2ccc(F)cc2)[nH]c2c(F)cc(F)cc12] | 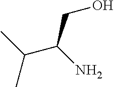 | ¹H NMR (300 MHz, Acetone- d₆) δ 10.80 (s, 1H), 7.98-7.68 (m, 2H), 7.47-7.11 (m, 3H), 7.05-6.64 (m, 1H), 5.50 (s, 1H), 3.85 (s, 2H), 3.66-3.40 (m, 5H), 3.25-2.82 (m, 2H), 1.90 (dq, J = 13.5, 6.9 Hz, 1H), 0.90 (dd, J = 11.1, 6.9 Hz, 6H); LCMS m/z 420.29 [M + H]⁺ |
| 270 | As for Compound 259 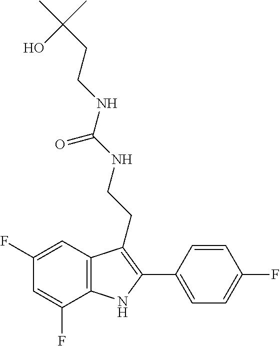 [SMILES: CC(C)(O)CCNC(=O)NCCc1c(-c2ccc(F)cc2)[nH]c2c(F)cc(F)cc12] | 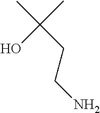 | ¹H NMR (300 MHz, Acetone- d₆) δ 7.99-7.62 (m, 2H), 7.44- 7.14 (m, 3H), 6.82 (ddd, J = 11.1, 9.6, 2.2 Hz, 1H), 3.45 (dd, J = 8.8, 6.4 Hz, 3H), 3.36- 3.09 (m, 4H), 3.14-2.90 (m, 3H), 1.82-1.51 (m, 2H), 1.18 (s, 6H); LCMS m/z 419.82 [M + H]⁺ |
| 271 | As for Compound 259 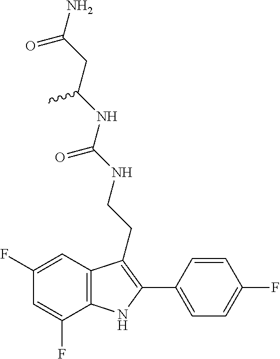 [SMILES: CC(CC(N)=O)NC(=O)NCCc1c(-c2ccc(F)cc2)[nH]c2c(F)cc(F)cc12] | 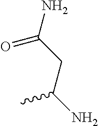 | ¹H NMR (300 MHz, Acetone- d₆) δ 10.72 (s, 1H), 7.87-7.74 (m, 2H), 7.36-7.20 (m, 3H), 7.01 (s, 1H), 6.81 (ddd, J = 11.1, 9.7, 2.2 Hz, 1H), 6.30 (s, 1H), 4.11 (h, J = 6.4 Hz, 1H), 3.50-3.39 (m, 2H), 3.06- 2.95 (m, 2H), 2.43 (dd, J = 14.8, 5.9 Hz, 1H), 2.28 (dd, J = 14.8, 6.2 Hz, 1H), 1.15 (d, J = 6.6 Hz, 3H); LCMS m/z 419 [M + H]⁺ |
| 272 | As for Compound 259 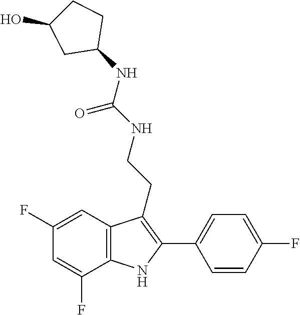 [SMILES: O=C(NCCc1c(-c2ccc(F)cc2)[nH]c2c(F)cc(F)cc12)N[C@@H]1CC[C@H](O)C1] | 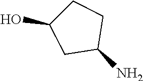 | ¹H NMR (300 MHz, Acetone- d₆) δ 8.54-8.08 (m, 1H), 7.94- 7.70 (m, 2H), 7.55-7.40 (m, 1H), 7.37-7.14 (m, 2H), 6.96- 6.68 (m, 1H), 4.35-4.12 (m, 1H), 4.06 (s, 1H), 3.54-3.38 (m, 2H), 3.10-2.95 (m, 3H), 1.99-1.80 (m, 2H), 1.67 (d, J = 3.2 Hz, 2H), 1.54-1.35 (m, 1H); LCMS m/z 417.8 [M + H]⁺ |
| 273 | As for Compound 259 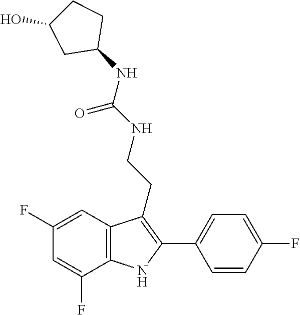 [SMILES: O=C(NCCc1c(-c2ccc(F)cc2)[nH]c2c(F)cc(F)cc12)N[C@@H]1CC[C@@H](O)C1] | 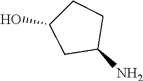 | ¹H NMR (300 MHz, Acetone- d₆) δ 8.02-7.72 (m, 2H), 7.41- 7.09 (m, 2H), 6.82 (ddd, J = 11.2, 9.7, 2.2 Hz, 1H), 4.27 (dd, J = 7.8, 4.9 Hz, 2H), 3.44 (t, J = 7.5 Hz, 2H), 3.00 (dd, J = 8.7, 6.4 Hz, 2H), 2.61-2.48 (m, 1H), 2.01-1.77 (m, 4H), 1.69-1.44 (m, 2H), 1.33 (ddd, J = 12.6, 9.0, 6.5 Hz, 2H); LCMS m/z 417.7 [M + H]⁺ |
| 274 | As for Compound 259 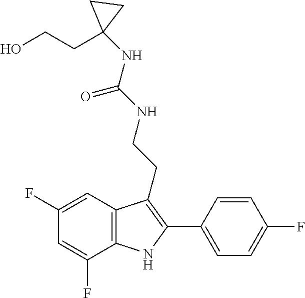 [SMILES: O=C(NCCc1c(-c2ccc(F)cc2)[nH]c2c(F)cc(F)cc12)NC1(CCO)CC1] | 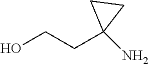 | ¹H NMR (300 MHz, Acetone- d₆) δ 10.75 (s, 1H), 7.79 (dd, J = 8.8, 5.4 Hz, 2H), 7.29 (t, J = 8.4 Hz, 3H), 6.83 (ddd, J = 11.1, 9.7, 2.2 Hz, 1H), 6.50 (s, 1H), 4.19 (s, 2H), 3.95 (s, 1H), 3.65 (s, 1H), 3.42 (q, J = 6.5 Hz, 2H), 3.05 (dd, J = 9.1, 6.3 Hz, 3H), 1.20 (q, J = 4.0 Hz, 2H), 0.98 (q, J = 4.0 Hz, 3H); LCMS m/z 417.8 [M + H]⁺ |
| 275 | As for Compound 259 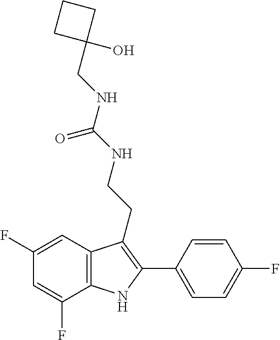 [SMILES: O=C(NCCc1c(-c2ccc(F)cc2)[nH]c2c(F)cc(F)cc12)NCC1(O)CCC1] | 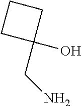 | ¹H NMR (300 MHz, Acetone- d₆) δ 10.75 (s, 1H), 7.81 (ddd, J = 8.1, 5.1, 2.4 Hz, 2H), 7.46- 7.10 (m, 3H), 6.82 (ddd, J = 11.6, 9.7, 2.2 Hz, 1H), 5.90 (s, 1H), 3.63-3.36 (m, 2H), 3.35 (s, 2H), 3.17-2.82 (m, 2H), 2.00-1.84 (m, 5H), 1.77-1.56 (m, 2H), 1.56-1.15 (m, 1H); LCMS m/z 417.83 [M + H]⁺ |
| 276 | As for Compound 259 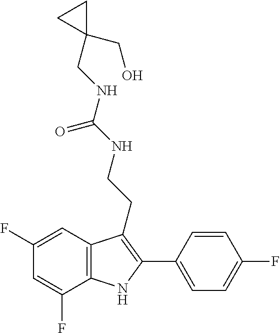 [SMILES: O=C(NCCc1c(-c2ccc(F)cc2)[nH]c2c(F)cc(F)cc12)NCC1(CO)CC1] | 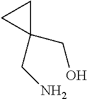 | ¹H NMR (300 MHz, Acetone- d₆) δ 7.98-7.66 (m, 2H), 7.40- 7.21 (m, 3H), 6.86-6.76 (m, 1H), 3.48 (dd, J = 8.7, 6.4 Hz, 2H), 3.16-2.96 (m, 5H), 0.64- 0.20 (m, 5H); LCMS m/z 417.8 [M + H]⁺ |
| 277 | As for Compound 259 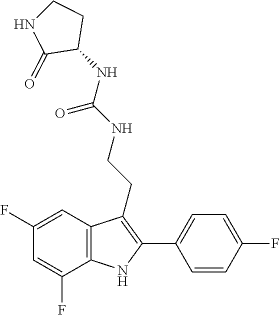 [SMILES: O=C(NCCc1c(-c2ccc(F)cc2)[nH]c2c(F)cc(F)cc12)N[C@H]1CCNC1=O] | 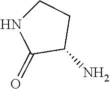 | ¹H NMR (300 MHz, Acetone- d₆) δ 10.72 (s, 1H), 7.89-7.66 (m, 2H), 7.44-7.21 (m, 3H), 7.05 (s, 1H), 6.81 (ddd, J = 11.0, 9.6, 2.2 Hz, 1H), 4.24 (t, J = 9.4 Hz, 1H), 3.58-3.42 (m, 2H), 3.42-3.31 (m, 2H), 3.18-2.90 (m, 2H), 2.56 (ddt, J = 12.1, 8.0, 3.9 Hz, 1H), 1.97- 1.70 (m, 1H); LCMS m/z 417.13 [M + H]⁺ |
| 278 | As for Compound 259 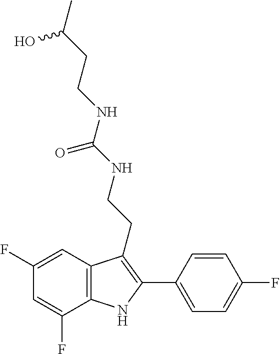 [SMILES: CC(O)CCNC(=O)NCCc1c(-c2ccc(F)cc2)[nH]c2c(F)cc(F)cc12] | 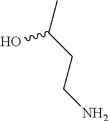 | ¹H NMR (300 MHz, Acetone- d₆) δ 10.70 (s, 1H), 7.96-7.70 (m, 2H), 7.46-7.18 (m, 3H), 6.82 (ddd, J = 11.1, 9.6, 2.2 Hz, 1H), 5.80 (s, 1H), 3.72 (ddd, J = 9.6, 6.3, 3.4 Hz, 1H), 3.51-3.31 (m, 3H), 3.14-2.86 (m, 5H), 1.67-1.44 (m, 1H), 1.36 (ddt, J = 13.7, 9.5, 4.8 Hz, 1H), 1.09 (d, J = 6.2 Hz, 3H); LCMS m/z 405.81 [M + H]⁺ |
| 279 | As for Compound 259 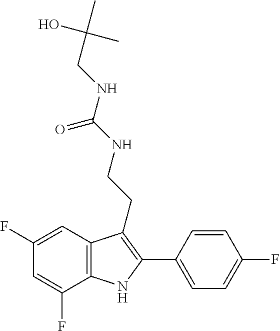 [SMILES: CC(C)(O)CNC(=O)NCCc1c(-c2ccc(F)cc2)[nH]c2c(F)cc(F)cc12] | 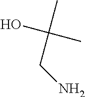 | ¹H NMR (300 MHz, Acetone- d₆) δ 7.92-7.64 (m, 2H), 7.46- 7.15 (m, 3H), 6.82 (ddd, J = 11.1, 9.7, 2.2 Hz, 1H), 3.61- 3.41 (m, 2H), 3.12 (s, 2H), 3.09-2.80 (m, 2H), 1.11 (s, 6H); LCMS m/z 405.85 [M + H]⁺ |
| 280 | As for Compound 259 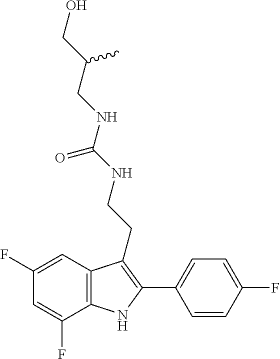 [SMILES: CC(CO)CNC(=O)NCCc1c(-c2ccc(F)cc2)[nH]c2c(F)cc(F)cc12] | 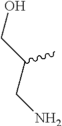 | ¹H NMR (300 MHz, Acetone- d₆) δ 7.99-7.70 (m, 2H), 7.47- 7.08 (m, 3H), 6.82 (ddd, J = 11.1, 9.7, 2.2 Hz, 1H), 3.61- 3.35 (m, 3H), 3.34-3.18 (m, 2H), 3.18-2.84 (m, 3H), 1.64 (qt, J = 6.8, 4.7 Hz, 1H), 0.82 (d, J = 6.9 Hz, 3H); LCMS m/z 406.13 [M + H]⁺ |
| 281 | As for Compound 259 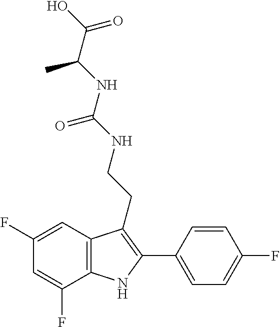 [SMILES: C[C@H](NC(=O)NCCc1c(-c2ccc(F)cc2)[nH]c2c(F)cc(F)cc12)C(=O)O] | 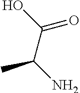 | ¹H NMR (300 MHz, Acetone- d₆) δ 7.93-7.62 (m, 2H), 7.40- 7.08 (m, 3H), 6.82 (ddd, J = 11.1, 9.7, 2.2 Hz, 1H), 6.02 (s, 1H), 4.33 (p, J = 7.0 Hz, 1H), 3.50 (s, 2H), 3.14-2.91 (m, 3H), 1.33 (d, J = 7.2 Hz, 3H); LCMS m/z 405.91 [M + H]⁺ |
| 282 | As for Compound 259 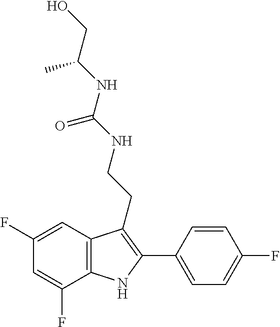 [SMILES: C[C@H](CO)NC(=O)NCCc1c(-c2ccc(F)cc2)[nH]c2c(F)cc(F)cc12] | 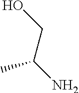 | ¹H NMR (300 MHz, Acetone- d₆) δ 8.00-7.72 (m, 2H), 7.40- 7.08 (m, 3H), 6.82 (ddd, J = 11.1, 9.7, 2.2 Hz, 1H), 3.79 (d, J = 7.3 Hz, 1H), 3.65-3.25 (m, 5H), 3.14-2.86 (m, 3H), 1.08 (d, J = 6.7 Hz, 3H); LCMS m/z 391.84 [M + H]⁺ |
| 283 | As for Compound 259 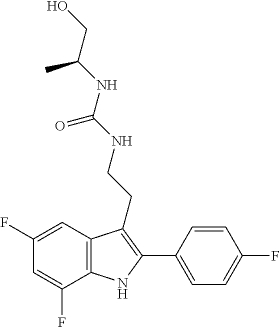 [SMILES: C[C@@H](CO)NC(=O)NCCc1c(-c2ccc(F)cc2)[nH]c2c(F)cc(F)cc12] | 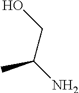 | ¹H NMR (300 MHz, Acetone- d₆) δ 10.70 (s, 1H), 7.93-7.64 (m, 2H), 7.40-7.14 (m, 3H), 6.93-6.60 (m, 1H), 5.68 (s, 1H), 5.36 (s, 1H), 4.13 (t, J = 5.3 Hz, 1H), 3.79 (dt, J = 12.1, 6.0 Hz, 1H), 3.44 (td, J = 5.2, 1.7 Hz, 2H), 3.30 (s, 2H), 3.11- 2.89 (m, 2H), 1.08 (d, J = 6.8 Hz, 3H); LCMS m/z 392.24 [M + H]⁺ |
| 284 | As for Compound 259 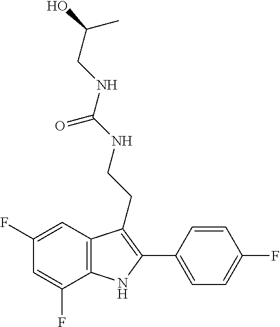 [SMILES: C[C@H](O)CNC(=O)NCCc1c(-c2ccc(F)cc2)[nH]c2c(F)cc(F)cc12] | 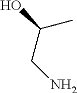 | ¹H NMR (300 MHz, Acetone- d₆) δ 7.99-7.62 (m, 2H), 7.42- 7.14 (m, 3H), 6.82 (ddd, J = 11.1, 9.7, 2.2 Hz, 1H), 5.81 (d, J = 43.8 Hz, 2H), 3.96-3.66 (m, 1H), 3.57-3.35 (m, 2H), 3.20 (dt, J = 13.8, 4.7 Hz, 1H), 3.09-2.89 (m, 4H), 1.07 (d, J = 6.3 Hz, 3H); LCMS m/z 391.94 [M + H]⁺ |
| 285 | As for Compound 259 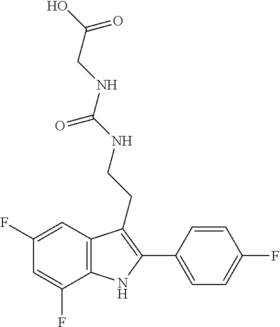 [SMILES: O=C(O)CNC(=O)NCCc1c(-c2ccc(F)cc2)[nH]c2c(F)cc(F)cc12] | 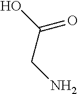 | ¹H NMR (300 MHz, Acetone- d₆) δ 10.75 (s, 1H), 7.94-7.72 (m, 2H), 7.55-7.35 (m, 3H), 7.36-7.08 (m, 1H), 6.85-6.75 (m, 1H), 6.10 (s, 1H), 5.90 (s, 1H), 3.99-3.78 (m, 2H), 3.64- 3.38 (m, 2H), 3.16-2.90 (m, 2H); LCMS m/z 391.77 [M + H]⁺ |
| 286 | As for Compound 259 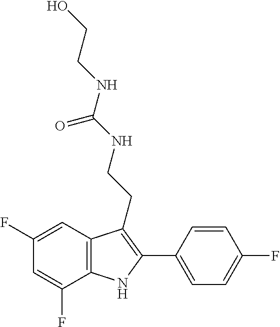 [SMILES: O=C(NCCO)NCCc1c(-c2ccc(F)cc2)[nH]c2c(F)cc(F)cc12] | 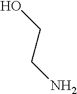 | ¹H NMR (300 MHz, Acetone- d₆) δ 8.31-8.05 (m, 1H), 7.89- 7.68 (m, 2H), 7.44-7.21 (m, 3H), 7.15-6.98 (m, 1H), 6.82 (ddd, J = 11.1, 9.7, 2.2 Hz, 1H), 3.71-3.54 (m, 2H), 3.54- 3.41 (m, 2H), 3.25 (dd, J = 5.7, 5.0 Hz, 2H), 3.10-2.84 (m, 2H); LCMS m/z 377.83 [M + H]⁺ |
| 287 | As for Compound 259 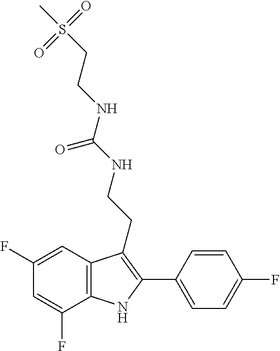 [SMILES: CS(=O)(=O)CCNC(=O)NCCc1c(-c2ccc(F)cc2)[nH]c2c(F)cc(F)cc12] | 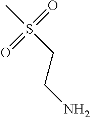 | ¹H NMR (300 MHz, Acetone- d₆) δ 7.93-7.66 (m, 2H), 7.42- 7.15 (m, 3H), 6.83 (ddd, J = 11.1, 9.7, 2.2 Hz, 1H), 5.95 (s, 1H), 5.76 (s, 1H), 3.70-3.55 (m, 2H), 3.24 (t, J = 6.4 Hz, 2H), 3.08-2.83 (m, 6H); LCMS m/z 439.85 [M + H]⁺ |
| 288 | As for Compound 259 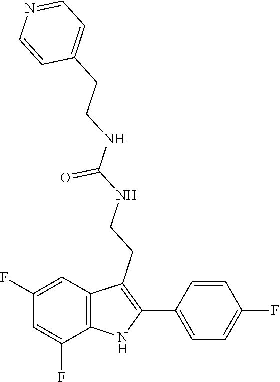 [SMILES: O=C(NCCc1ccncc1)NCCc1c(-c2ccc(F)cc2)[nH]c2c(F)cc(F)cc12] | 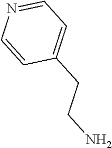 | ¹H NMR (300 MHz, Acetone- d₆) δ 8.92-8.63 (m, 2H), 7.80 (dt, J = 5.3, 3.8 Hz, 4H), 7.29 (dtd, J = 8.9, 7.1, 6.6, 2.2 Hz, 3H), 6.83 (ddd, J = 11.1, 9.7, 2.2 Hz, 1H), 3.54 (t, J = 6.8 Hz, 2H), 3.44 (dd, J = 8.7, 6.4 Hz, 2H), 3.14-2.83 (m, 4H); LCMS m/z 439.26 [M + H]⁺ |
| 289 | As for Compound 259 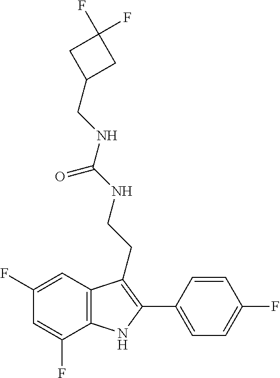 [SMILES: O=C(NCCc1c(-c2ccc(F)cc2)[nH]c2c(F)cc(F)cc12)NCC1CC(F)(F)C1] | 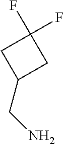 | ¹H NMR (300 MHz, Acetone- d₆) δ 7.93-7.68 (m, 2H), 7.46- 7.12 (m, 3H), 6.82 (ddd, J = 11.1, 9.7, 2.2 Hz, 1H), 3.44 (t, J = 7.6 Hz, 2H), 3.26 (d, J = 5.4 Hz, 2H), 3.08-2.97 (m, 2H), 2.56 (ddd, J = 13.5, 7.5, 5.5 Hz, 2H), 2.45-2.13 (m, 3H); LCMS m/z 437.77 [M + H]⁺ |
| 290 | As for Compound 259 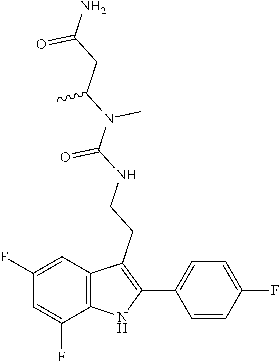 [SMILES: CC(CC(N)=O)N(C)C(=O)NCCc1c(-c2ccc(F)cc2)[nH]c2c(F)cc(F)cc12] | 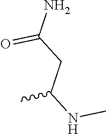 | ¹H NMR (300 MHz, Acetone- d₆) δ 10.71 (s, 1H), 7.89-7.76 (m, 2H), 7.39-7.21 (m, 3H), 7.06 (s, 1H), 6.81 (ddd, J = 11.1, 9.7, 2.2 Hz, 1H), 6.21 (d, J = 22.5 Hz, 2H), 4.74-4.59 (m, 1H), 3.48-3.39 (m, 2H), 3.07-2.96 (m, 2H), 2.69 (s, 3H), 2.56-2.21 (m, 2H), 1.12 (d, J = 6.8 Hz, 3H); LCMS m/z 433.14 [M + H]⁺ |
| 291 | As for Compound 259 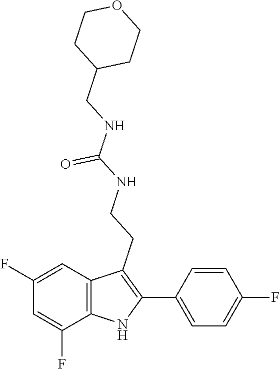 [SMILES: O=C(NCCc1c(-c2ccc(F)cc2)[nH]c2c(F)cc(F)cc12)NCC1CCOCC1] | 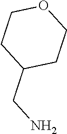 | ¹H NMR (300 MHz, Acetone- d₆) δ 7.81 (ddt, J = 8.6, 5.4, 2.7 Hz, 2H), 7.42-7.19 (m, 3H), 6.82 (ddd, J = 11.6, 9.6, 2.2 Hz, 1H), 3.95-3.73 (m, 2H), 3.45 (dd, J = 8.6, 6.3 Hz, 2H), 3.27 (td, J = 11.7, 2.1 Hz, 3H), 3.16-2.90 (m, 5H), 1.71-1.48 (m, 3H), 1.33-1.06 (m, 2H); LCMS m/z 431.81 [M + H]⁺ |
| 292 | As for Compound 259 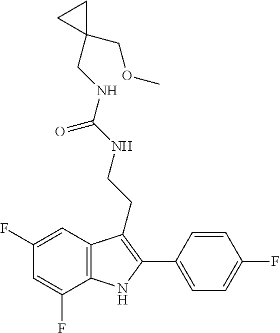 [SMILES: COCC1(CNC(=O)NCCc2c(-c3ccc(F)cc3)[nH]c3c(F)cc(F)cc23)CC1] | 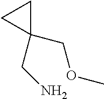 | ¹H NMR (300 MHz, Acetone- d₆) δ 10.75 (s, 1H), 8.03-7.60 (m, 2H), 7.42-7.19 (m, 3H), 6.82 (ddd, J = 11.1, 9.7, 2.2 Hz, 1H), 3.55-3.36 (m, 4H), 3.30- 3.10 (m, 7H), 3.03-2.88 (m, 2H), 0.74-0.25 (m, 4H); LCMS m/z 431.81 [M + H]⁺ |
| 293 | As for Compound 259 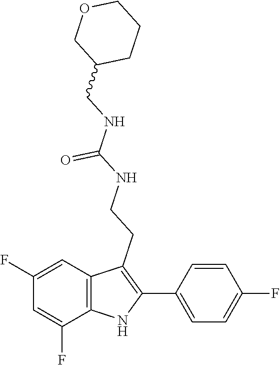 [SMILES: O=C(NCCc1c(-c2ccc(F)cc2)[nH]c2c(F)cc(F)cc12)NCC1CCCOC1] | 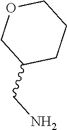 | ¹H NMR (300 MHz, Acetone- d₆) δ 8.00-7.68 (m, 2H), 7.41- 7.14 (m, 3H), 6.82 (ddd, J = 11.1, 9.7, 2.2 Hz, 1H), 4.02- 3.65 (m, 2H), 3.63-3.42 (m, 2H), 3.31 (ddd, J = 11.2, 10.1, 3.1 Hz, 2H), 3.26-2.80 (m, 6H), 1.91-1.02 (m, 5H); LCMS m/z 432.28 [M + H]⁺ |
| 294 | As for Compound 259 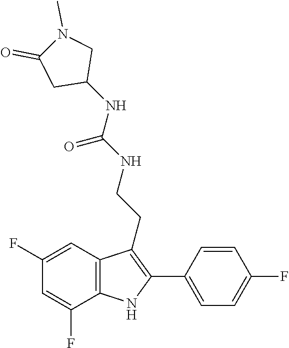 [SMILES: CN1CC(NC(=O)NCCc2c(-c3ccc(F)cc3)[nH]c3c(F)cc(F)cc23)CC1=O] | 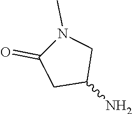 | ¹H NMR (300 MHz, Acetone- d₆) δ 7.98-7.63 (m, 2H), 7.44- 7.21 (m, 3H), 6.83 (ddd, J = 11.1, 9.7, 2.2 Hz, 1H), 4.49- 4.23 (m, 1H), 3.69 (dd, J = 10.2, 6.9 Hz, 1H), 3.50-3.28 (m, 2H), 3.20 (dd, J = 10.2, 4.0 Hz, 1H), 3.14-2.95 (m, 2H), 2.77 (d, J = 1.6 Hz, 3H), 2.63 (ddq, J = 16.9, 8.3, 0.8 Hz, 1H), 2.23-2.10 (m, 1H); LCMS m/z 430.76 [M + H]⁺ |
| 295 | As for Compound 259 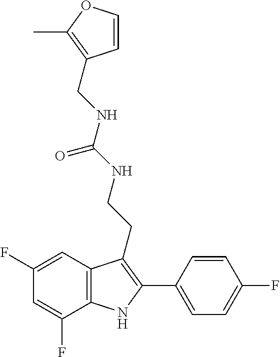 [SMILES: Cc1occc1CNC(=O)NCCc1c(-c2ccc(F)cc2)[nH]c2c(F)cc(F)cc12] | 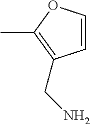 | ¹H NMR (300 MHz, Acetone- d₆) δ 7.96-7.68 (m, 2H), 7.47- 7.09 (m, 4H), 6.82 (ddd, J = 11.1, 9.7, 2.2 Hz, 1H), 6.31 (d, J = 1.9 Hz, 1H), 4.09 (s, 2H), 3.46 (t, J = 7.7 Hz, 2H), 3.17- 2.83 (m, 4H), 2.24 (s, 3H); LCMS m/z 428.28 [M + H]⁺ |
| 296 | As for Compound 259 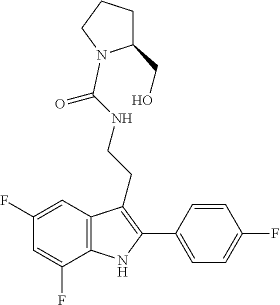 [SMILES: O=C(NCCc1c(-c2ccc(F)cc2)[nH]c2c(F)cc(F)cc12)N1CCC[C@H]1CO] | 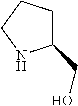 | ¹H NMR (300 MHz, Acetone- d₆) δ 10.71 (s, 1H), 7.81 (dd, J = 8.8, 5.4 Hz, 2H), 7.34-7.22 (m, 3H), 6.82 (ddd, J = 11.1, 9.7, 2.2 Hz, 1H), 3.94 (s, 1H), 3.52-3.39 (m, 4H), 3.34-3.20 (m, 2H), 3.05 (dd, J = 8.4, 6.3 Hz, 2H), 2.04-1.74 (m, 3H), 1.63 (dt, J = 9.8, 5.3 Hz, 1H); LCMS m/z 417.96 [M + H]⁺ |
| 297 | As for Compound 259 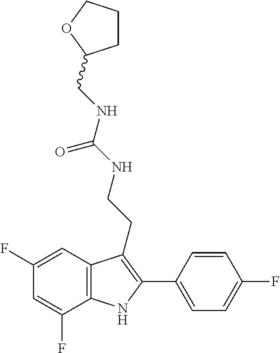 [SMILES: O=C(NCCc1c(-c2ccc(F)cc2)[nH]c2c(F)cc(F)cc12)NCC1CCCO1] | 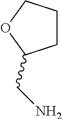 | ¹H NMR (300 MHz, Acetone- d₆) δ 8.12-7.57 (m, 2H), 7.46- 7.09 (m, 3H), 6.82 (ddd, J = 11.1, 9.7, 2.2 Hz, 1H), 3.90- 3.73 (m, 2H), 3.73-3.54 (m, 1H), 3.46 (t, J = 7.6 Hz, 2H), 3.30-2.80 (m, 6H), 1.97-1.63 (m, 3H), 1.64-1.45 (m, 1H); LCMS m/z 417.8 [M + H]⁺ |
| 298 | As for Compound 259 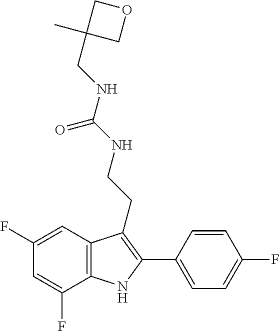 [SMILES: CC1(CNC(=O)NCCc2c(-c3ccc(F)cc3)[nH]c3c(F)cc(F)cc23)COC1] | 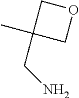 | ¹H NMR (300 MHz, Acetone- d₆) δ 11.35 (s, 1H), 7.93-7.60 (m, 2H), 7.42-7.12 (m, 3H), 6.84 (ddd, J = 11.6, 9.7, 2.2 Hz, 1H), 4.30 (d, J = 10.7 Hz, 2H), 4.07 (d, J = 10.8 Hz, 2H), 3.55 (d, J = 5.2 Hz, 3H), 3.30 (d, J = 12.4 Hz, 1H), 3.23- 2.95 (m, 4H), 0.95 (s, 3H); LCMS m/z 418.27 [M + H]⁺ |
| 299 | As for Compound 259 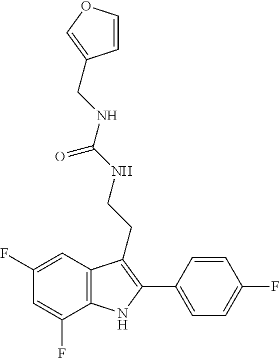 [SMILES: O=C(NCCc1c(-c2ccc(F)cc2)[nH]c2c(F)cc(F)cc12)NCc1ccoc1] | 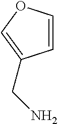 | ¹H NMR (300 MHz, Acetone- d₆) δ 7.93-7.68 (m, 2H), 7.54- 7.42 (m, 1H), 7.41-7.14 (m, 3H), 6.82 (ddd, J = 11.1, 9.7, 2.2 Hz, 1H), 6.42 (dd, J = 1.8, 0.8 Hz, 1H), 4.18 (s, 2H), 3.73- 3.35 (m, 2H), 3.21-2.83 (m, 3H); LCMS m/z 414.28 [M + H]⁺ |
| 300 | As for Compound 259 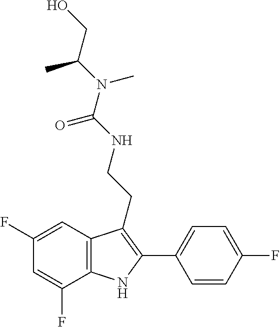 [SMILES: C[C@@H](CO)N(C)C(=O)NCCc1c(-c2ccc(F)cc2)[nH]c2c(F)cc(F)cc12] | 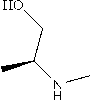 | ¹H NMR (300 MHz, Acetone- d₆) δ 8.11-7.65 (m, 2H), 7.44- 7.15 (m, 3H), 6.98-6.60 (m, 1H), 4.31 (dp, J = 13.7, 7.1 Hz, 1H), 3.70-3.36 (m, 4H), 3.25- 2.95 (m, 2H), 2.73 (s, 3H), 1.07 (d, J = 6.9 Hz, 3H); LCMS m/z 406.16 [M + H]⁺ |
| 301 | As for Compound 259 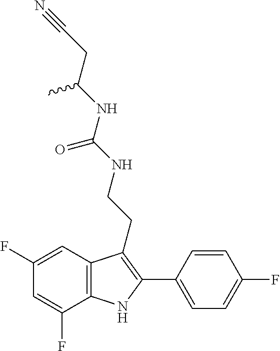 [SMILES: CC(CC#N)NC(=O)NCCc1c(-c2ccc(F)cc2)[nH]c2c(F)cc(F)cc12] | 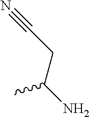 | ¹H NMR (300 MHz, Acetone- d₆) δ 8.05-7.68 (m, 2H), 7.29 (dtd, J = 8.8, 7.0, 2.2 Hz, 3H), 6.83 (ddd, J = 11.1, 9.7, 2.2 Hz, 1H), 5.95-5.48 (m, 2H), 4.04 (p, J = 6.2 Hz, 1H), 3.46 (p, J = 7.2 Hz, 2H), 3.01 (dd, J = 7.8, 6.8 Hz, 2H), 2.88-2.54 (m, 2H), 1.24 (d, J = 6.8 Hz, 3H); LCMS m/z 401.28 [M + H]⁺ |
| 302 | As for Compound 259 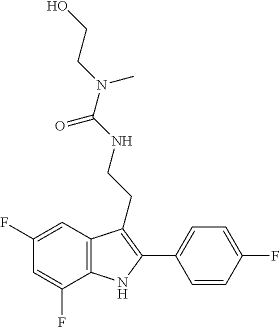 [SMILES: CN(CCO)C(=O)NCCc1c(-c2ccc(F)cc2)[nH]c2c(F)cc(F)cc12] | 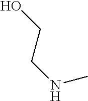 | ¹H NMR (300 MHz, Acetone- d₆) δ 10.71 (s, 1H), 7.87-7.75 (m, 2H), 7.38-7.20 (m, 3H), 6.82 (ddd, J = 11.1, 9.6, 2.2 Hz, 1H), 6.09 (s, 1H), 3.74- 3.59 (m, 2H), 3.52-3.41 (m, 2H), 3.36 (t, J = 5.4 Hz, 2H), 3.13-2.97 (m, 2H), 2.89 (s, 3H); LCMS m/z 392.16 [M + H]⁺ |
| 303 | As for Compound 259 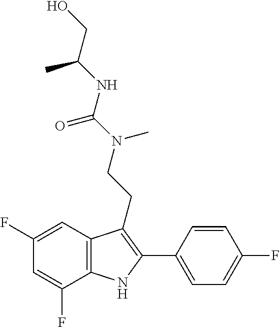 [SMILES: C[C@@H](CO)NC(=O)N(C)CCc1c(-c2ccc(F)cc2)[nH]c2c(F)cc(F)cc12] | 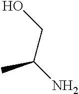 | ¹H NMR (300 MHz, Acetone- d₆) δ 10.72 (s, 1H), 7.86-7.74 (m, 2H), 7.38-7.23 (m, 3H), 6.82 (ddd, J = 11.6, 9.7, 2.1 Hz, 1H), 3.84 (s, 1H), 3.64- 3.53 (m, 2H), 3.43 (d, J = 5.4 Hz, 2H), 3.06 (t, J = 7.5 Hz, 2H), 2.83 (s, 3H), 1.05 (d, J = 6.7 Hz, 3H); LCMS m/z 406.24 [M + H]⁺ |

1. Compound 303 was synthesized using the conditions as for compound 259, but compound 428 was used in place of S19.

### Compound 304

### tert-butyl-N-[3-amino-1-[2-[5,7-difluoro-2-(4-fluorophenyl)-1H-indol-3-yl]ethylcarbamoyl]-3-oxo-propyl]carbamate (304)

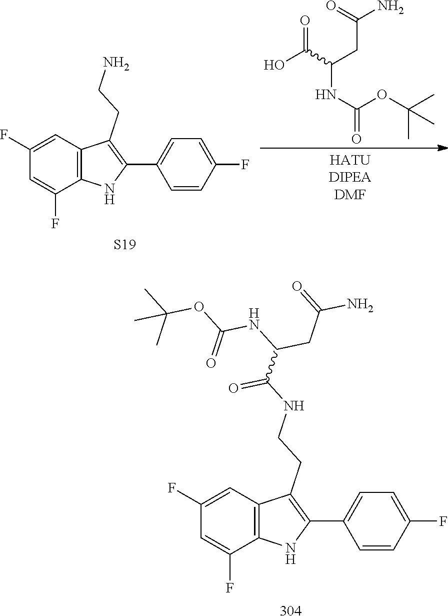 [SMILES: NCCc1c(-c2ccc(F)cc2)[nH]c2c(F)cc(F)cc12 ; CC(C)(C)OC(=O)NC(CC(N)=O)C(=O)O ; "S19" ; CC(C)(C)OC(=O)NC(CC(N)=O)C(=O)NCCc1c(-c2ccc(F)cc2)[nH]c2c(F)cc(F)cc12 ; "304"]

### Preparation of tert-butyl N-[3-amino-1-[2-[5,7-difluoro-2-(4-fluorophenyl)-1H-indol-3-yl]ethylcarbamoyl]-3-oxo-propyl]carbamate (304)

To a solution of 2-[5,7-difluoro-2-(4-fluorophenyl)-1H-indol-3-yl]ethanamine (Trifluoroacetate salt) S19 (50 mg, 0.1092 mmol) in DMF (2 mL) was added 4-amino-2-(tert-butoxycarbonylamino)-4-oxo-butanoic acid (25 mg, 0.108 mmol), HATU (40 mg, 0.105 mmol), and DIPEA (50 μL, 0.2871 mmol). The mixture was allowed to stir at room temperature overnight. The mixture was then purified by reversed-phase HPLC (Method: C18 Waters Sunfire column (30×150 mm, 5 micron). Gradient: MeCN in H₂O with 0.1% trifluoroacetic acid) to afford the product tert-butyl N-[3-amino-1-[2-[5,7-difluoro-2-(4-fluorophenyl)-1H-indol-3-yl]ethylcarbamoyl]-3-oxo-propyl]carbamate (2.6 mg, 8%). ¹H NMR (300 MHz, Acetone-d₆) δ 10.76 (s, 1H), 7.80 (ddt, J=8.7, 5.5, 3.8 Hz, 2H), 7.41-7.22 (m, 4H), 6.83 (ddt, J=11.2, 9.6, 2.4 Hz, 1H), 4.36 (s, 1H), 3.60-3.47 (m, 3H), 3.13-2.96 (m, 2H), 2.82-2.52 (m, 1H), 2.10-2.07 (m, 1H), 1.39 (s, 9H). LCMS m/z 505.2 [M+H]⁺.

### Compounds 305 and 306

### (4R)—N-[2-[5,7-difluoro-2-(4-fluorophenyl)-1H-indol-3-yl]ethyl]-2-oxo-oxazolidine-4-carboxamide (305) and (4R)—N-[2-[5,7-difluoro-2-(4-fluorophenyl)-1H-indol-3-yl]ethyl]-2-oxo-oxazolidine-4-carboxamide (306)

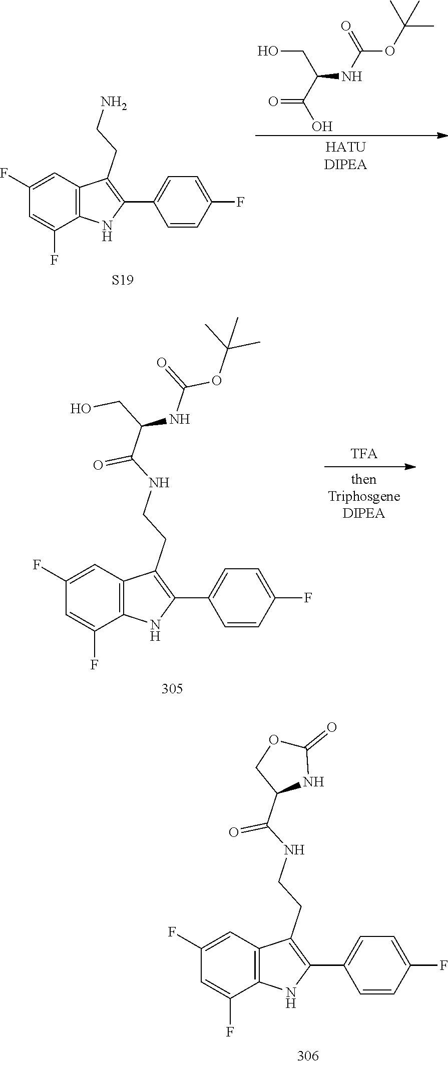 [SMILES: NCCc1c(-c2ccc(F)cc2)[nH]c2c(F)cc(F)cc12 ; CC(C)(C)OC(=O)N[C@H](CO)C(=O)O ; "S19" ; CC(C)(C)OC(=O)N[C@H](CO)C(=O)NCCc1c(-c2ccc(F)cc2)[nH]c2c(F)cc(F)cc12 ; O=C1N[C@@H](C(=O)NCCc2c(-c3ccc(F)cc3)[nH]c3c(F)cc(F)cc23)CO1]

### Step 1. Synthesis of N-[(1R)-2-[2-[5,7-difluoro-2-(4-fluorophenyl)-1H-indol-3-yl]ethylamino]-1-(hydroxymethyl)-2-oxo-ethyl]carbamate (305)

To a solution of 2-[5,7-difluoro-2-(4-fluorophenyl)-1H-indol-3-yl]ethanamine (Trifluoroacetate salt) S19 (40 mg, 0.087 mmol) and DIPEA (33.3 μL, 0.191 mmol) dissolved in DMF (2 mL) was added (2R)-2-(tert-butoxycarbonylamino)-3-hydroxy-propanoic acid (60 mg, 0.2924 mmol) and HATU (40 mg, 0.105 mmol). The mixture was allowed to stir at room temperature overnight. The mixture was then purified by reversed-phase HPLC (Method: C18 Waters Sunfire column (30×150 mm, 5 micron). Gradient: MeCN in H₂O with 0.1% trifluoroacetic acid) to afford tert-butyl N-[(1R)-2-[2-[5,7-difluoro-2-(4-fluorophenyl)-1H-indol-3-yl]ethylamino]-1-(hydroxymethyl)-2-oxo-ethyl]carbamate (11.8 mg, 30%). ¹H NMR (300 MHz, Acetone-d₆) δ 7.88-7.60 (m, 3H), 7.43-7.06 (m, 3H), 6.83 (dddd, J=11.1, 9.7, 6.0, 2.2 Hz, 1H), 4.20-4.02 (m, 1H), 3.98-3.62 (m, 2H), 3.64-3.48 (m, 2H), 3.06 (q, J=7.0 Hz, 2H), 1.52-1.14 (m, 9H). LCMS m/z 478.2 [M+H]⁺.

### Step 2. Synthesis of (4R)—N-[2-[5,7-difluoro-2-(4-fluorophenyl)-1H-indol-3-yl]ethyl]-2-oxo-oxazolidine-4-carboxamide (306)

To a solution of tert-butyl N-[(1R)-2-[2-[5,7-difluoro-2-(4-fluorophenyl)-1H-indol-3-yl]ethylamino]-1-(hydroxymethyl)-2-oxo-ethyl]carbamate 305 (15 mg, 0.030 mmol) in dichloromethane was added trifluoroacetic acid (20 μL, 0.2596 mmol) and the solution was stirred at room temperature for 3 hours. The reaction was concentrated and redissolved in dichloromethane (5 mL). To the solution was added triphosgene (15 mg, 0.05055 mmol) and diisopropylethylamine (20 μL, 0.1148 mmol) and stirred overnight. Reaction was concentrated and dissolved in DMSO (2 mL). The mixture was then purified by reversed-phase HPLC (Method: C18 Waters Sunfire column (30×150 mm, 5 micron). Gradient: MeCN in H₂O with 0.1% trifluoroacetic acid) to afford the product (4R)—N-[2-[5,7-difluoro-2-(4-fluorophenyl)-1H-indol-3-yl]ethyl]-2-oxo-oxazolidine-4-carboxamide (4.7 mg, 33%). ¹H NMR (300 MHz, Acetone-d₆) δ 7.94-7.63 (m, 2H), 7.30 (dtd, J=8.9, 5.8, 2.9 Hz, 2H), 6.85 (t, J=10.5 Hz, 1H), 4.55 (dd, J=9.4, 8.4 Hz, 1H), 4.34 (ddd, J=9.4, 5.2, 1.7 Hz, 1H), 4.19 (d, J=5.2 Hz, 1H), 3.83-3.66 (m, 2H), 3.66-3.50 (m, 1H), 3.07 (td, J=8.7, 7.5, 4.1 Hz, 2H). LCMS m/z 404.22 [M+H]⁺.

### Compounds 307-417

Compounds 307-417 (see Table 11) were prepared from intermediate S19 using the appropriate reagents, using the amide formation methods as described for compounds 304, and 306 (using coupling reagents such as HATU, post amide formation modifications). Carboxylic acids were prepared by methods described above or obtained from commercial sources. Any modifications to methods are noted in Table 11 and accompanying footnotes.

TABLE 11
Method of preparation, structure, physicochemical data for compounds 307-417

| Com- | | | ¹H NMR; LCMS m/z |
|---|---|---|---|
| pound | Method/Product | Acid Reagent | [M + H]⁺. |
| 307 | As for Compound 304 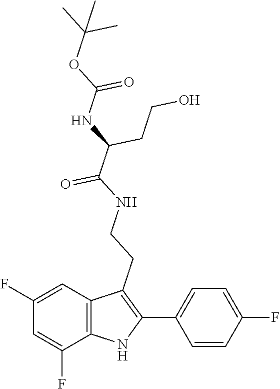 [SMILES: CC(C)(C)OC(=O)N[C@@H](CCO)C(=O)NCCc1c(-c2ccc(F)cc2)[nH]c2c(F)cc(F)cc12] | 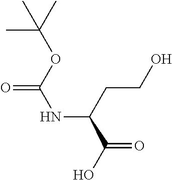 | ¹H NMR (300 MHz, Acetone-d₆) δ, 10.8 (s, 1H), 7.80 (ddd, J = 8.5, 5.4, 2.6 Hz, 2H), 7.43-7.19 (m, 3H), 6.96-6.78 (m, 1H), 6.20 (d, J = 7.8 Hz, 1H), 4.37-4.09 (m, 1H), 3.72-3.44 (m, 4H), 3.05 (dd, J = 9.3, 6.4 Hz, 2H), 2.16-1.99 (m, 2H), 2.02-1.83 (m, 1H), 1.85- 1.57 (m, 1H), 1.40 (d, J = 1.9 Hz, 9H); LCMS m/z 492.24 [M + H]⁺. |
| 308 | As for Compound 304 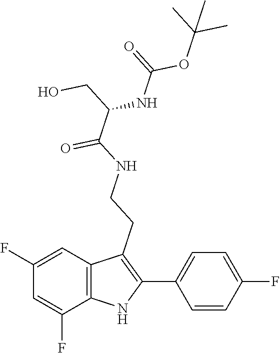 [SMILES: CC(C)(C)OC(=O)N[C@@H](CO)C(=O)NCCc1c(-c2ccc(F)cc2)[nH]c2c(F)cc(F)cc12] | 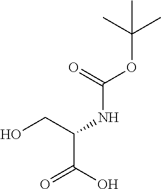 | ¹H NMR (300 MHz, Acetone-d₆) δ 10.8 (s, 1H), 7.93-7.73 (m, 2H), 7.41- 7.08 (m, 3H), 6.91-6.65 (m, 1H), 4.12 (s, 1H), 3.96-3.63 (m, 2H), 3.66-3.43 (m, 2H), 3.06 (q, J = 8.1 Hz, 2H), 1.42 (d, J = 7.8 Hz, 9H); LCMS m/z 478.29 [M + H]⁺. |
| 309 | As for Compound 304 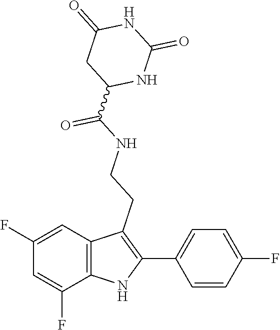 [SMILES: O=C1CC(C(=O)NCCc2c(-c3ccc(F)cc3)[nH]c3c(F)cc(F)cc23)NC(=O)N1] | 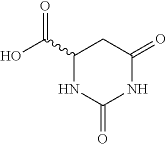 [SMILES: O=C1CC(C(=O)O)NC(=O)N1] | ¹H NMR (300 MHz, Acetone-d₆) δ 10.75 (s, 1H), 7.84-7.74 (m, 2H), 7.55 (s, 1H), 7.36-7.22 (m, 3H), 6.89-6.75 (m, 1H), 3.76 (s, 1H), 3.57-3.48 (m, 2H), 3.24-3.13 (m, 1H), 3.09- 2.95 (m, 2H), 2.38 (s, 2H), 2.25-2.14 (m, 1H). LCMS m/z 431.2 [M + H]⁺. |
| 310 | As for Compound 304² 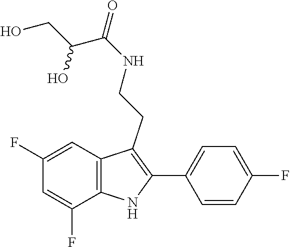 [SMILES: O=C(NCCc1c(-c2ccc(F)cc2)[nH]c2c(F)cc(F)cc12)C(O)CO] | 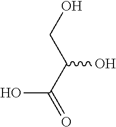 | ¹H NMR (300 MHz, Acetone-d₆) δ 7.81 (dddd, J = 17.3, 6.7, 5.3, 3.1 Hz, 3H), 7.42-7.17 (m, 3H), 6.98- 6.68 (m, 1H), 4.17-3.98 (m, 1H), 3.75 (dd, J = 11.0, 4.4 Hz, 1H), 3.65 (d, J = 6.0 Hz, 4H), 3.27-2.85 (m, 1H); LCMS m/z 379.1 [M + H]⁺. |
| 311 | As for Compound 304 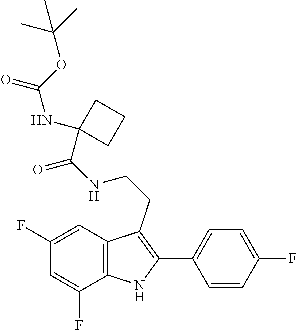 [SMILES: CC(C)(C)OC(=O)NC1(C(=O)NCCc2c(-c3ccc(F)cc3)[nH]c3c(F)cc(F)cc23)CCC1] | 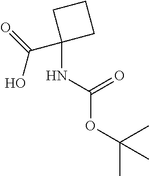 | ¹H NMR (300 MHz, Acetone-d₆) δ 7.95-7.72 (m, 2H), 7.40 (s,1H), 7.37-7.22 (m, 2H), 6.83 (ddd, J = 11.1, 9.6, 2.2 Hz, 1H), 3.72-3.44 (m, 2H), 3.04 (s, 2H), 2.74- 2.50 (m, 2H), 2.15 (s, 1H), 1.92 (ddd, J = 16.1, 8.9, 4.2 Hz, 3H), 1.36 (s, 9H); LCMS m/z 488.24 [M + H]⁺. |
| 312 | As for Compound 304 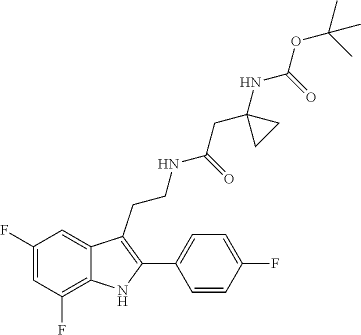 [SMILES: CC(C)(C)OC(=O)NC1(CC(=O)NCCc2c(-c3ccc(F)cc3)[nH]c3c(F)cc(F)cc23)CC1] | 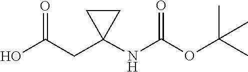 | ¹H NMR (300 MHz, Acetone-d₆) δ 7.98-7.65 (m, 2H), 7.38-7.19 (m, 3H), 6.83 (ddd, J = 11.1, 9.6, 2.2 Hz, 1H), 3.49 (ddd, J = 9.6, 7.8, 6.0 Hz, 3H), 3.15-2.96 (m, 2H), 2.41 (s, 2H), 1.36 (s, 9H), 0.93-0.58 (m, 4H); LCMS m/z 488.3 [M + H]⁺. |
| 313 | As for Compound 304¹ 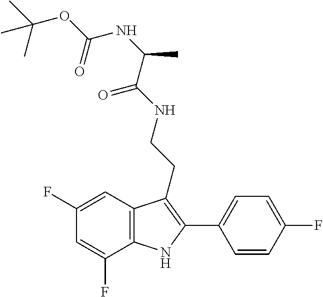 [SMILES: C[C@H](NC(=O)OC(C)(C)C)C(=O)NCCc1c(-c2ccc(F)cc2)[nH]c2c(F)cc(F)cc12] | 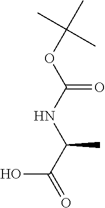 | ¹H NMR (300 MHz, Acetone-d₆) δ 7.94-7.70 (m, 2H), 7.44-7.16 (m, 3H), 6.98-6.67 (m, 1H), 4.27- 3.92 (m, 1H), 3.79-3.42 (m, 2H), 3.04 (q, J = 7.9 Hz, 2H), 1.40 (s, 3H), 1.26 (d, J = 7.1 Hz, 9H); LCMS m/z 462.3 [M + H]⁺. |
| 314 | As for Compound 304 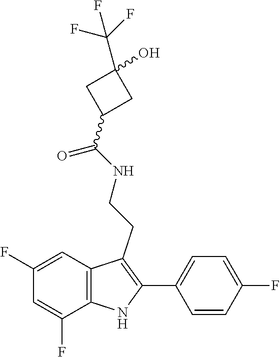 [SMILES: O=C(NCCc1c(-c2ccc(F)cc2)[nH]c2c(F)cc(F)cc12)C1CC(O)(C(F)(F)F)C1] | 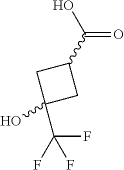 [SMILES: O=C(O)C1CC(O)(C(F)(F)F)C1] | ¹H NMR (300 MHz, Acetone-d₆) δ 7.99-7.65 (m, 2H), 7.35-7.12 (m, 3H), 6.84 (ddd, J = 11.1, 9.7, 2.2 Hz, 1H), 3.69-3.36 (m, 2H), 3.23-2.88 (m, 2H), 2.84- 2.69 (m, 1H), 2.69-2.50 (m, 2H), 2.37 (dddd, J = 13.7, 8.1, 2.9, 1.4 Hz, 2H); LCMS m/z 457.2 [M + H]⁺. |
| 315 | As for Compound 304² 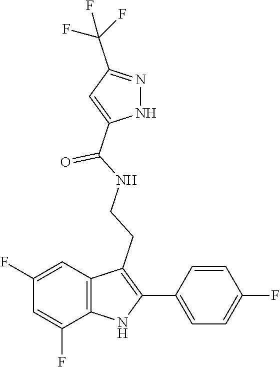 [SMILES: O=C(NCCc1c(-c2ccc(F)cc2)[nH]c2c(F)cc(F)cc12)c1cc(C(F)(F)F)n[nH]1] | 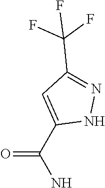 [SMILES: NC(=O)c1cc(C(F)(F)F)n[nH]1] | ¹H NMR (300 MHz, Acetone-d₆) δ 7.89-7.61 (m, 2H), 7.32 (dd, J = 9.3, 2.2 Hz, 1H), 7.25-7.12 (m, 2H), 7.05 (s, 1H), 6.85 (ddd, J = 11.1, 9.6, 2.2 Hz, 1H), 3.66 (td, J = 7.3, 6.1 Hz, 2H), 3.16 (dd, J = 8.1, 6.7 Hz, 2H); LCMS m/z 453.19 [M + H]⁺. |
| 316 | As for Compound 304² 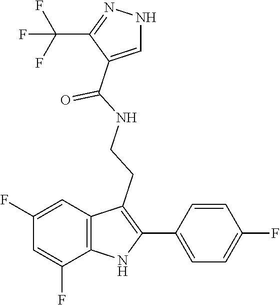 [SMILES: O=C(NCCc1c(-c2ccc(F)cc2)[nH]c2c(F)cc(F)cc12)c1c[nH]nc1C(F)(F)F] | 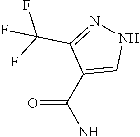 [SMILES: NC(=O)c1c[nH]nc1C(F)(F)F] | ¹H NMR (300 MHz, Acetone-d₆) δ 8.31 (dq, J = 42.6, 1.1 Hz, 1H), 7.92-7.73 (m, 2H), 7.37-7.08 (m, 3H), 6.84 (ddd, J = 11.1, 9.7, 2.2 Hz, 1H), 3.66 (d, J = 6.1 Hz, 5H), 3.23-3.05 (m, 2H); LCMS m/z 453.09 [M + H]⁺. |
| 317 | As for Compound 304 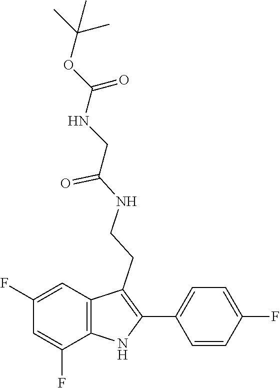 [SMILES: CC(C)(C)OC(=O)NCC(=O)NCCc1c(-c2ccc(F)cc2)[nH]c2c(F)cc(F)cc12] | 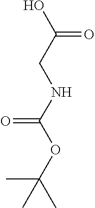 | ¹H NMR (300 MHz, Acetone-d₆) δ 7.86-7.71 (m, 2H), 7.36-7.18 (m, 3H), 6.95-6.76 (m, 1H), 3.66 (s, 2 H), 3.53 (q, J = 6.9 Hz, 2H), 3.15-2.95 (m, 2H), 1.41 (s, 9H); LCMS m/z 448.3 [M + H]⁺. |
| 318 | As for Compound 304 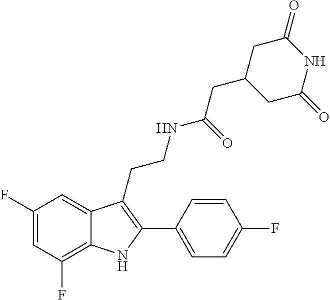 [SMILES: O=C(CC1CC(=O)NC(=O)C1)NCCc1c(-c2ccc(F)cc2)[nH]c2c(F)cc(F)cc12] | 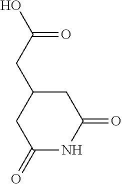 [SMILES: O=C(O)CC1CC(=O)NC(=O)C1] | ¹H NMR (300 MHz, Acetone-d₆) δ 10.75 (s, 1H), 9.46 (s, 1H), 7.86-7.73 (m, 2H), 7.48 (s, 1H), 7.36-7.22 (m, 3H), 6.83 (ddd, J = 11.1, 9.7, 2.2 Hz, 1H), 3.59-3.46 (m, 2H), 3.10-2.99 (m, 2H), 2.70-2.46 (m, 3H), 2.40- 2.28 (m, 2H), 2.25 (d, J = 6.6 Hz, 2H); LCMS m/z 444.2 [M + H]⁺ |
| 319 | As for Compound 304 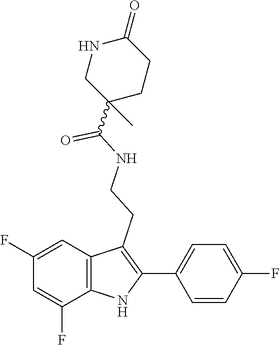 [SMILES: CC1(C(=O)NCCc2c(-c3ccc(F)cc3)[nH]c3c(F)cc(F)cc23)CCC(=O)NC1] | 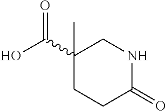 | ¹H NMR (300 MHz, Acetone-d₆) δ 7.91-7.73 (m, 3H), 7.55 (s, 1H), 7.41-7.14 (m, 3H), 6.96-6.60 (m, 1H), 3.92-3.40 (m, 3H), 3.34- 2.85 (m, 3H), 2.28 (d, J = 62.9 Hz, 3H), 1.83 (d, J = 11.5 Hz, 1H), 1.22 (s, 3H); LCMS m/z 430.3 [M + H]⁺. |
| 320 | As for Compound 304² 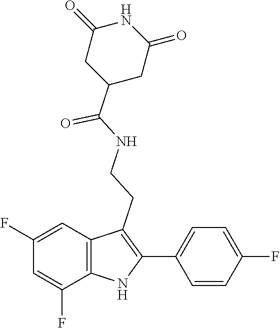 [SMILES: O=C1CC(C(=O)NCCc2c(-c3ccc(F)cc3)[nH]c3c(F)cc(F)cc23)CC(=O)N1] | 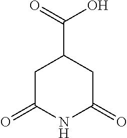 [SMILES: O=C1CC(C(=O)O)CC(=O)N1] | ¹H NMR (300 MHz, Acetone-d₆) δ 7.98-7.72 (m, 2H), 7.53 (s, 1H), 7.42-7.16 (m, 3H), 6.84 (ddd, J = 11.1, 9.7, 2.2 Hz, 1H), 3.69-3.32 (m, 2H), 3.25-2.80 (m, 3H), 2.73-2.41 (m, 3H); LCMS m/z 430.19 [M + H]⁺. |
| 321 | As for Compound 304 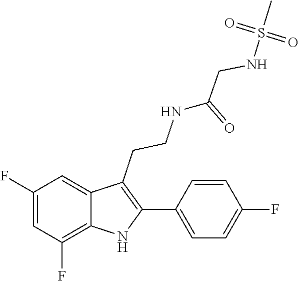 [SMILES: CS(=O)(=O)NCC(=O)NCCc1c(-c2ccc(F)cc2)[nH]c2c(F)cc(F)cc12] | 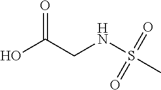 | LCMS m/z 426.1 [M + H]⁺ |
| 322 | As for Compound 304² 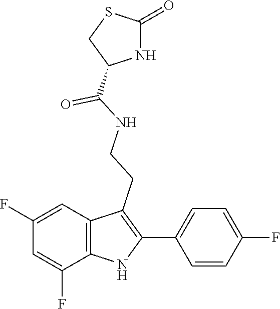 [SMILES: O=C1N[C@H](C(=O)NCCc2c(-c3ccc(F)cc3)[nH]c3c(F)cc(F)cc23)CS1] | 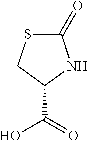 | ¹H NMR (300 MHz, Acetone-d₆) δ 7.96-7.60 (m, 3H), 7.40-7.16 (m, 3H), 6.84 (ddd, J = 11.1, 9.7, 2.2 Hz, 1H), 4.35 (ddd, J = 8.5, 5.1, 1.7 Hz, 1H), 3.73 (dd, J = 11.2, 8.5 Hz, 1H), 3.66- 3.53 (m, 2H), 3.53-3.40 (m, 1H), 3.21-2.83 (m, 2H); LCMS m/z 420.11 [M + H]⁺. |
| 323 | As for Compound 306 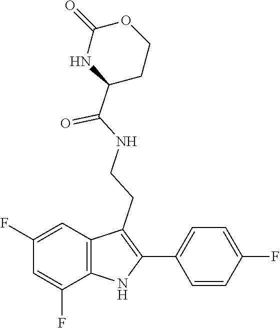 [SMILES: O=C1N[C@H](C(=O)NCCc2c(-c3ccc(F)cc3)[nH]c3c(F)cc(F)cc23)CCO1] | | ¹H NMR (300 MHz, Acetone-d₆) δ 8.00-7.67 (m, 2H), 7.50-7.18 (m, 3H), 6.95-6.61 (m, 1H), 6.46 (s, 1H), 4.27-3.95 (m, 4H), 3.69-3.46 (m, 2H), 3.22- 2.92 (m, 2H), 1.57-1.09 (m, 2H); LCMS m/z 418.17 [M + H]⁺. |
| 324 | As for Compound 304² 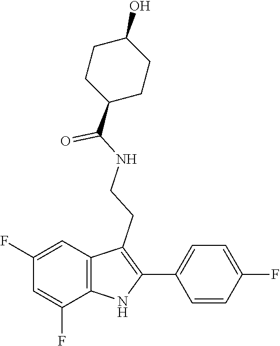 [SMILES: O=C(NCCc1c(-c2ccc(F)cc2)[nH]c2c(F)cc(F)cc12)[C@H]1CC[C@@H](O)CC1] | 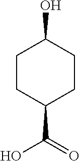 | ¹H NMR (300 MHz, Acetone-d₆) δ 7.93-7.72 (m, 2H), 7.40-7.11 (m, 3H), 6.83 (ddd, J = 11.1, 9.7, 2.2 Hz, 1H), 3.91 (dq, J = 4.9, 2.4 Hz, 1H), 3.85-3.41 (m, 2H), 3.30-2.91 (m, 2H), 2.20 (tt, J = 11.0, 3.4 Hz, 1H), 2.04-1.85 (m, 2H), 1.76 (dt, J = 13.1, 4.0 Hz, 2H), 1.50 (tdd, J = 12.2, 5.6, 3.1 Hz, 4H); LCMS m/z 417.23 [M + H]⁺. |
| 325 | As for Compound 304² 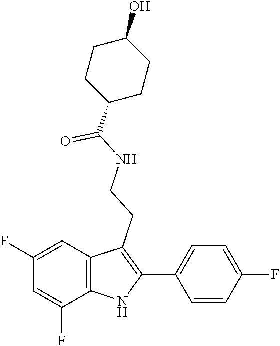 [SMILES: O=C(NCCc1c(-c2ccc(F)cc2)[nH]c2c(F)cc(F)cc12)[C@H]1CC[C@H](O)CC1] | 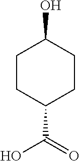 | ¹H NMR (300 MHz, Acetone-d₆) δ 10.74 (s, 1H), 7.85-7.74 (m, 2H), 7.39- 7.23 (m, 4H), 6.83 (ddd, J = 11.0, 9.7, 2.2 Hz, 1H), 3.54- 3.38 (m, 3H), 3.03 (dd, J = 8.5, 6.4 Hz, 2H), 2.28-1.06 (m, 8H); LCMS m/z 417.23 [M + H]⁺. |
| 326 | As for Compound 304² 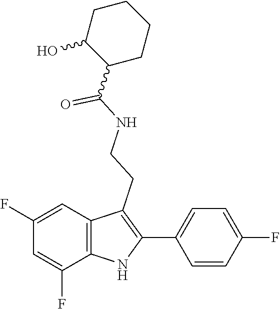 [SMILES: O=C(NCCc1c(-c2ccc(F)cc2)[nH]c2c(F)cc(F)cc12)C1CCCCC1O] | 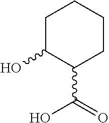 | ¹H NMR (300 MHz, Acetone-d₆) δ 10.74 (s, 1H), 7.79 (ddt, J = 8.5, 5.3, 2.6 Hz, 2H), 7.54 (s, 1H), 7.36- 7.22 (m, 3H), 6.83 (ddd, J = 11.1, 9.7, 2.2 Hz, 1H), 3.96 (s, 1H), 3.58-3.44 (m, 2H), 3.04 (t, J = 7.5 Hz, 2H), 2.28- 2.16 (m, 1H), 1.86 (td, J = 12.4, 3.5 Hz, 1H), 1.81- 1.56 (m, 3H), 1.52-1.14 (m, 4H); LCMS m/z 417.26 [M + H]⁺. |
| 327 | As for Compound 304² 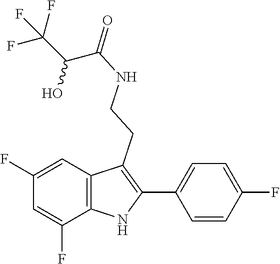 [SMILES: O=C(NCCc1c(-c2ccc(F)cc2)[nH]c2c(F)cc(F)cc12)C(O)C(F)(F)F] | 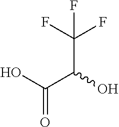 | ¹H NMR (300 MHz, Acetone-d₆) δ 7.89-7.67 (m, 2H), 7.44-7.16 (m, 3H), 6.84 (ddd, J = 11.1, 9.7, 2.2 Hz, 1H), 4.57 (q, J = 7.7 Hz, 1H), 3.76-3.46 (m, 2H), 3.35-2.89 (m, 2H); LCMS m/z 417.16 [M + H]⁺. |
| 328 | As for Compound 306² 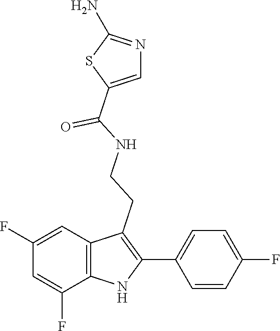 [SMILES: Nc1ncc(C(=O)NCCc2c(-c3ccc(F)cc3)[nH]c3c(F)cc(F)cc23)s1] | 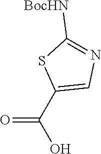 [SMILES: CC(C)(C)OC(=O)Nc1ncc(C(=O)O)s1] | ¹H NMR (300 MHz, Acetone-d₆) δ 7.91-7.73 (m, 2H), 7.65 (s, 1H), 7.42-7.15 (m, 3H), 6.85 (ddd, J = 11.1, 9.7, 2.2 Hz, 1H), 3.80-3.51 (m, 2H), 3.36-2.97 (m, 2H); LCMS m/z 417.1 [M + H]⁺. |
| 329 | As for Compound 304² 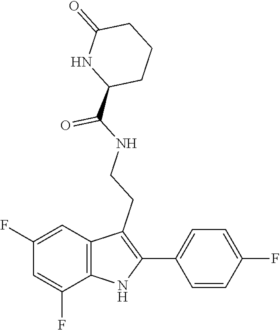 [SMILES: O=C1CCC[C@@H](C(=O)NCCc2c(-c3ccc(F)cc3)[nH]c3c(F)cc(F)cc23)N1] | 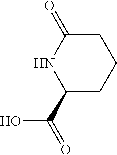 | ¹H NMR (300 MHz, Acetone-d₆) δ 7.89-7.68 (m, 3H), 7.42-7.12 (m, 4H), 6.84 (ddd, J = 11.1, 9.6, 2.2 Hz, 1H), 4.00 (s, 1H), 3.56 (q, J = 7.4, 7.0 Hz, 2H), 3.06 (dd, J = 9.4, 6.3 Hz, 2H), 2.26 (t, J =6.4 Hz, 2H), 2.02- 1.58 (m, 4H); LCMS m/z 416.27 [M + H]⁺. |
| 330 | As for Compound 304² 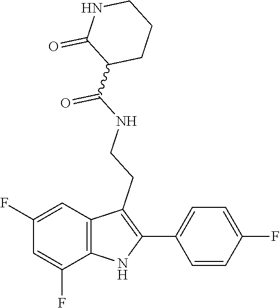 [SMILES: O=C1NCCCC1C(=O)NCCc1c(-c2ccc(F)cc2)[nH]c2c(F)cc(F)cc12] | 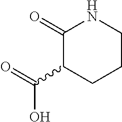 | ¹H NMR (300 MHz, Acetone-d₆) δ 7.93-7.54 (m, 2H), 7.44-7.08 (m, 5H), 6.83 (ddd, J = 11.1, 9.7, 2.2 Hz, 1H), 3.53 (q, J = 6.7 Hz, 2H), 3.30 (t, J = 5.9 Hz, 2H), 3.04 (t, J = 7.6 Hz, 2H), 2.14 (d, J = 8.7 Hz, 1H), 2.01- 1.47 (m, 3H); LCMS m/z 413.99 [M + H]⁺. |
| 331 | As for Compound 304² 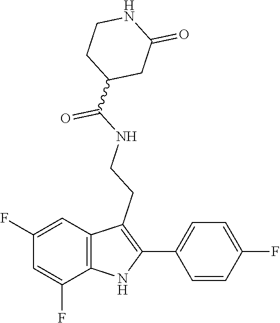 [SMILES: O=C1CC(C(=O)NCCc2c(-c3ccc(F)cc3)[nH]c3c(F)cc(F)cc23)CCN1] | 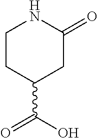 | ¹H NMR (300 MHz, Acetone-d₆) δ 7.87-7.63 (m, 4H), 7.38-7.02 (m, 3H), 6.84 (ddd, J = 11.0, 9.7, 2.2 Hz, 1H), 3.70-3.36 (m, 3H), 3.05 (t, J =7.5 Hz, 2H), 2.95- 2.73 (m, 1H), 2.57 (qd, J = 18.0, 7.0 Hz, 2H), 2.05-1.64 (m, 3H); LCMS m/z 416.24 [M + H]⁺. |
| 332 | As for Compound 304 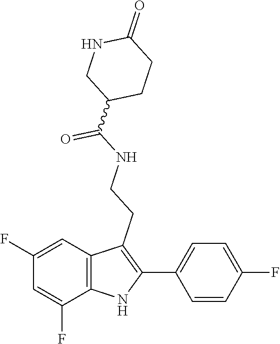 [SMILES: O=C1CCC(C(=O)NCCc2c(-c3ccc(F)cc3)[nH]c3c(F)cc(F)cc23)CN1] | 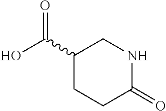 | ¹H NMR (300 MHz, Acetone-d₆) δ 7.89-7.70 (m, 4H), 7.41-7.18 (m, 3H), 6.85 (ddd, J = 11.1, 9.7, 2.2 Hz, 1H), 3.71-3.34 (m, 4H), 3.04 (dd, J = 16.2, 8.8 Hz, 2H), 2.81 (d, J = 13.5 Hz, 2H), 2.64 (s, 2H); LCMS m/z 416.2 [M + H]⁺ |
| 333 | As for Compound 304 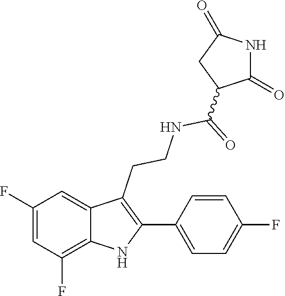 [SMILES: O=C1CC(C(=O)NCCc2c(-c3ccc(F)cc3)[nH]c3c(F)cc(F)cc23)C(=O)N1] | 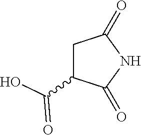 | ¹H NMR (300 MHz, Acetone-d₆) δ 10.76 (s, 1H), 10.12 (s, 1H), 7.87-7.74 (m, 2H), 7.77-7.65 (m, 1H), 7.38-7.22 (m, 3H), 6.84 (ddd, J = 11.1, 9.7, 2.2 Hz, 1H), 3.76 (dd, J = 9.3, 4.6 Hz, 1H), 3.71-3.43 (m, 2H), 3.18-3.02 (m, 2H), 3.01- 2.70 (m, 1H); LCMS m/z 416.2 [M + H]⁺ |
| 334 | As for Compound 304² 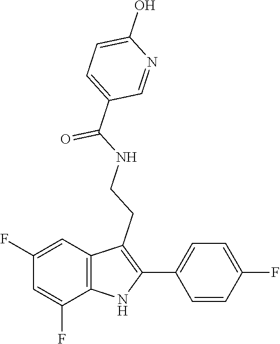 [SMILES: O=C(NCCc1c(-c2ccc(F)cc2)[nH]c2c(F)cc(F)cc12)c1ccc(O)nc1] | 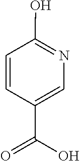 | ¹H NMR (300 MHz, Acetone-d₆) δ 8.08 (d, J = 2.6 Hz, 1H), 7.95-7.85 (m, 2H), 7.77 (ddd, J = 8.5, 5.3, 2.7 Hz, 2H), 7.42-7.19 (m, 3H), 6.93-6.75 (m, 1H), 6.49 (d, J = 9.6 Hz, 1H), 3.63 (dt, J = 7.8, 6.1 Hz, 3H), 3.31-3.01 (m, 3H); LCMS m/z 412.14 [M + H]⁺. |
| 335 | As for Compound 304 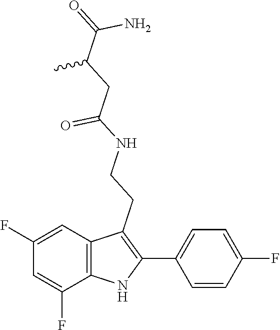 [SMILES: CC(CC(=O)NCCc1c(-c2ccc(F)cc2)[nH]c2c(F)cc(F)cc12)C(N)=O] | 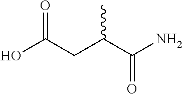 | ¹H NMR (300 MHz, Acetone-d₆) δ 7.98-7.59 (m, 2H), 7.43 (s, 1H), 7.28 (ddt, J = 12.0, 9.7, 2.6 Hz, 3H), 7.04 (s, 1H), 6.82 (ddd, J = 11.0, 9.7, 2.2 Hz, 1H), 6.39 (s, 1H), 3.70-3.40 (m, 2H), 3.01 (t, J = 7.7 Hz, 2H), 2.95- 2.72 (m, 1H), 2.50 (dd, J = 14.7, 7.7 Hz, 1H), 2.36-2.12 (m, 1H), 1.09 (d, J = 7.0 Hz, 3H); LCMS m/z 404.2 [M + H]⁺. |
| 336 | Compound 306 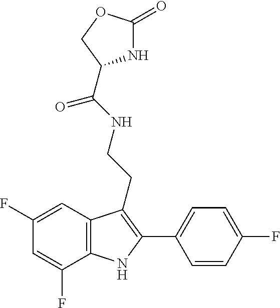 [SMILES: O=C1N[C@H](C(=O)NCCc2c(-c3ccc(F)cc3)[nH]c3c(F)cc(F)cc23)CO1] | 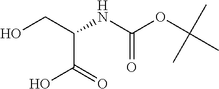 | ¹H NMR (300 MHz, Acetone-d₆) δ 10.83 (s, 1H), 7.87-7.74 (m, 2H), 7.36- 7.23 (m, 3H), 6.94-6.77 (m, 2H), 4.54 (dd, J = 9.4, 8.4 Hz, 1H), 4.34 (ddd, J = 9.4, 5.3, 1.6 Hz, 1H), 4.17 (dd, J = 8.4, 5.2 Hz, 1H), 3.62- 3.49 (m, 2H), 3.08-3.00 (m, 1H); LCMS m/z 404.17 [M + H]⁺. |
| 337 | As for Compound 304² 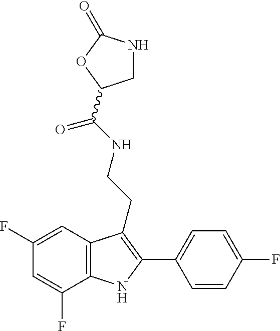 [SMILES: O=C1NCC(C(=O)NCCc2c(-c3ccc(F)cc3)[nH]c3c(F)cc(F)cc23)O1] | 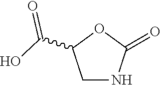 | ¹H NMR (300 MHz, Acetone-d₆) δ 10.75 (s, 1H), 7.87-7.74 (m, 3H), 7.39- 7.21 (m, 3H), 6.83 (ddd, J = 11.1, 9.7, 2.2 Hz, 1H), 6.67 (s, 1H), 4.90 (dd, J = 9.6, 5.9 Hz, 1H), 3.90-3.78 (m, 1H), 3.72-3.48 (m, 3H), 3.15-3.04 (m, 2H); LCMS m/z 404.12 [M + H]⁺. |
| 338 | As for Compound 304² 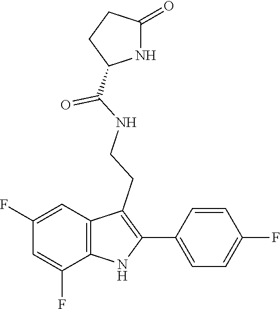 [SMILES: O=C1CC[C@@H](C(=O)NCCc2c(-c3ccc(F)cc3)[nH]c3c(F)cc(F)cc23)N1] | 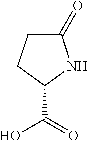 | ¹H NMR (300 MHz, Acetone-d₆) δ 7.96-7.72 (m, 2H), 7.44-7.15 (m, 4H), 6.84 (ddd, J =11.1, 9.7, 2.2 Hz, 1H), 4.14 (dd, J = 8.4, 4.6 Hz, 1H), 3.80-3.34 (m, 2H), 3.22-2.86 (m, 2H), 2.40 (t, J = 10.2 Hz, 1H), 2.36-2.20 (m, 2H), 1.99 (dd, J = 8.8, 4.8 Hz, 5H); LCMS m/z 402.21 [M + H]⁺. |
| 339 | As for Compound 304 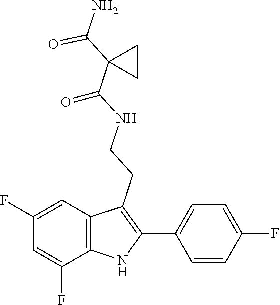 [SMILES: NC(=O)C1(C(=O)NCCc2c(-c3ccc(F)cc3)[nH]c3c(F)cc(F)cc23)CC1] | 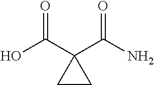 | ¹H NMR (300 MHz, Acetone-d₆) δ 8.02-7.63 (m, 2H), 7.44-7.08 (m, 3H), 6.84 (ddd, J = 11.1, 9.7, 2.2 Hz, 1H), 3.74-3.41 (m, 2H), 3.32-2.95 (m, 2H), 1.60- 1.22 (m, 4H); LCMS m/z 402.2 [M + H]⁺. |
| 340 | As for Compound 304 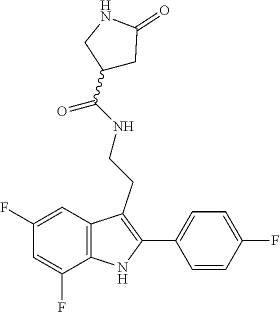 [SMILES: O=C1CC(C(=O)NCCc2c(-c3ccc(F)cc3)[nH]c3c(F)cc(F)cc23)CN1] | 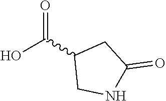 | ¹H NMR (300 MHz, Acetone-d₆) δ 8.04-7.68 (m, 2H), 7.37-7.21 (m, 3H), 6.84 (ddd, J = 11.0, 9.7, 2.2 Hz, 1H), 3.73-3.29 (m, 4H), 3.29-3.12 (m, 1H), 3.05 (dd, J = 8.5, 6.4 Hz, 2H), 2.58- 2.16 (m, 2H); LCMS m/z 402.1 [M + H]⁺. |
| 341 | As for Compound 304² 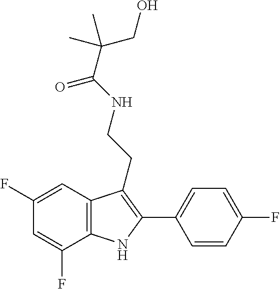 [SMILES: CC(C)(CO)C(=O)NCCc1c(-c2ccc(F)cc2)[nH]c2c(F)cc(F)cc12] | 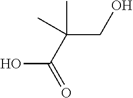 | ¹H NMR (300 MHz, Acetone-d₆) δ 7.94-7.69 (m, 2H), 7.40-7.19 (m, 3H), 6.97-6.63 (m, 1H), 3.63- 3.43 (m, 4H), 3.21-2.86 (m, 2H), 1.10 (s, 6H); LCMS m/z 391.23 [M + H]⁺. |
| 342 | As for Compound 304 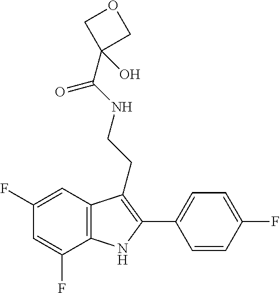 [SMILES: O=C(NCCc1c(-c2ccc(F)cc2)[nH]c2c(F)cc(F)cc12)C1(O)COC1] | 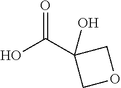 | LCMS m/z 391.1 [M + H]⁺. |
| 343 | As for Compound 304 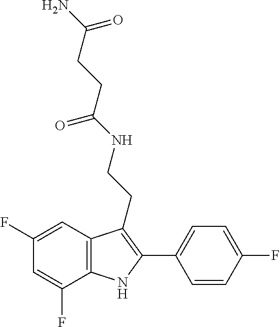 [SMILES: NC(=O)CCC(=O)NCCc1c(-c2ccc(F)cc2)[nH]c2c(F)cc(F)cc12] | 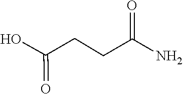 | ¹H NMR (300 MHz, Acetone-d₆) δ 7.96-7.67 (m, 2H), 7.53-7.37 (m, 1H), 7.28 (qt, J = 6.8, 2.1 Hz, 2H), 7.00-6.74 (m, 1H), 4.01 (dd, J = 8.4, 7.0 Hz, 1H), 3.60- 3.34 (m, 2H), 3.22-2.76 (m, 4H), 2.64-2.31 (m, 2H), 2.33-2.16 (m, 1H); LCMS m/z 390.13 [M + H]⁺. |
| 344 | As for Compound 304 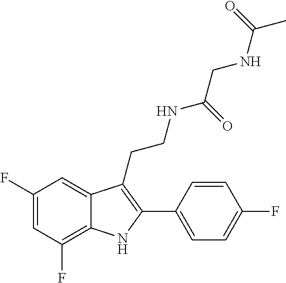 [SMILES: CC(=O)NCC(=O)NCCc1c(-c2ccc(F)cc2)[nH]c2c(F)cc(F)cc12] | 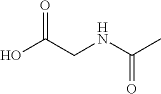 | LCMS m/z 390.1 [M + H]⁺ |
| 345 | As for Compound 304 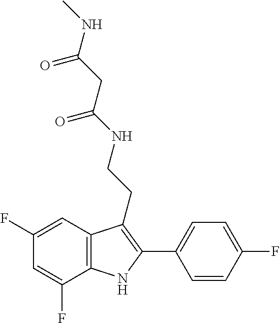 [SMILES: CNC(=O)CC(=O)NCCc1c(-c2ccc(F)cc2)[nH]c2c(F)cc(F)cc12] | 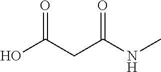 | ¹H NMR (300 MHz, Acetone-d₆) δ 7.92-7.55 (m, 3H), 7.46-7.14 (m, 3H), 6.84 (ddd, J = 11.1, 9.7, 2.2 Hz, 1H), 6.29 (s, 2H), 3.79- 3.46 (m, 2H), 3.18 (s, 2H), 3.12-2.97 (m, 2H), 2.77 (d, J = 3.7 Hz, 3H); LCMS m/z 390.2 [M + H]⁺. |
| 346 | As for Compound 304 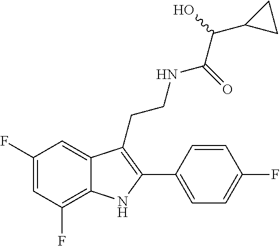 [SMILES: O=C(NCCc1c(-c2ccc(F)cc2)[nH]c2c(F)cc(F)cc12)C(O)C1CC1] | 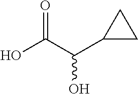 | ¹H NMR (300 MHz, Acetone-d₆) δ 10.75 (s, 1H), 7.90-7.77 (m, 2H), 7.53 (s, 1H), 7.37 (dd, J = 9.4, 2.2 Hz, 1H), 7.33-7.23 (m, 2H), 6.84 (ddd, J = 11.1, 9.7, 2.2 Hz, 1H), 4.69-4.43 (m, 1H), 3.68 (d, J = 6.1 Hz, 1H), 3.64-3.51 (m, 2H), 1.21- 1.00 (m, 1H), 0.57-0.24 (m, 4H); LCMS m/z 389.2 [M + H]⁺. |
| 347 | As for Compound 304 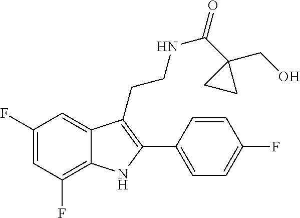 [SMILES: O=C(NCCc1c(-c2ccc(F)cc2)[nH]c2c(F)cc(F)cc12)C1(CO)CC1] | 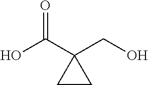 | ¹H NMR (300 MHz, Acetone-d₆) δ 7.96-7.74 (m, 2H), 7.37 (dd, J = 9.4, 2.2 Hz, 1H), 7.34-7.11 (m, 2H), 6.92-6.70 (m, 1H), 4.55 (s, 1H), 3.74-3.44 (m, 3H), 3.27-2.90 (m, 2H), 1.37- 1.18 (m, 1H), 1.18-0.93 (m, 2H), 0.63 (q, J = 3.8 Hz, 1H); LCMS m/z 389.3 [M + H]⁺ |
| 348 | As for Compound 304 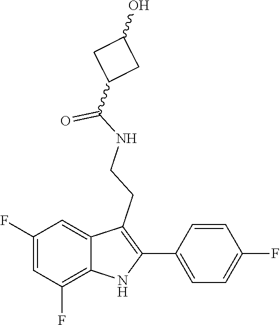 [SMILES: O=C(NCCc1c(-c2ccc(F)cc2)[nH]c2c(F)cc(F)cc12)C1CC(O)C1] | 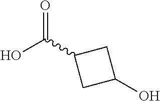 | LCMS m/z 389.2 [M + H]⁺ |
| 349 | As for Compound 304² 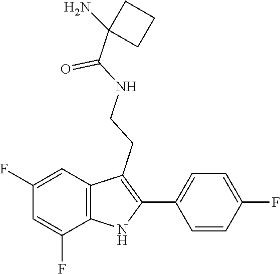 [SMILES: NC1(C(=O)NCCc2c(-c3ccc(F)cc3)[nH]c3c(F)cc(F)cc23)CCC1] | 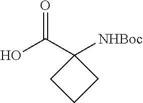 [SMILES: CC(C)(C)OC(=O)NC1(C(=O)O)CCC1] | LCMS m/z 389.2 [M + H]⁺. |
| 350 | As for Compound 304 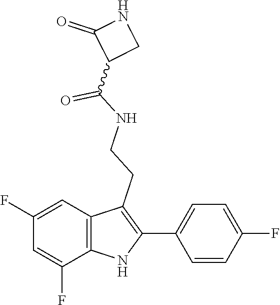 [SMILES: O=C(NCCc1c(-c2ccc(F)cc2)[nH]c2c(F)cc(F)cc12)C1CNC1=O] | 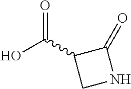 | ¹H NMR (300 MHz, Acetone-d₆) δ 7.96-7.65 (m, 3H), 7.46-7.17 (m, 4H), 6.83 (ddd, J = 11.1, 9.7, 2.2 Hz, 1H), 4.01 (dd, J = 5.8, 2.7 Hz, 1H), 3.70-3.44 (m, 2H), 3.25-3.01 (m, 5H); LCMS m/z 388.2 [M + H]⁺. |
| 351 | As for Compound 304 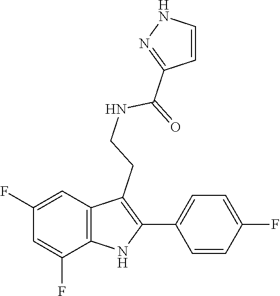 [SMILES: O=C(NCCc1c(-c2ccc(F)cc2)[nH]c2c(F)cc(F)cc12)c1cc[nH]n1] | 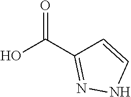 | ¹H NMR (300 MHz, Acetone-d₆) δ 7.98-7.64 (m, 4H), 7.41 (dd, J = 9.5, 2.2 Hz, 1H), 7.36-7.05 (m, 2H), 6.96-6.64 (m, 3H), 3.89- 3.64 (m, 2H), 3.38-3.01 (m, 2H); LCMS m/z 385.2 [M + H]⁺ |
| 352 | As for Compound 304² 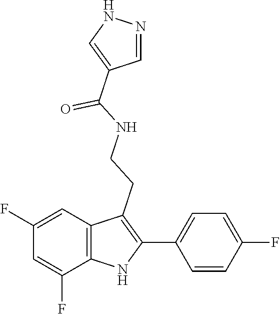 [SMILES: O=C(NCCc1c(-c2ccc(F)cc2)[nH]c2c(F)cc(F)cc12)c1cn[nH]c1] | 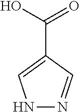 | ¹H NMR (300 MHz, Acetone-d₆) δ 10.74 (s, 1H), 8.20-7.65 (m, 5H), 7.38- 7.19 (m, 3H), 6.84 (ddd, J = 11.5, 9.6, 2.2 Hz, 1H), 3.70- 3.58 (m, 2H), 3.17-3.06 (m, 2H); LCMS m/z 385.16 [M + H]⁺. |
| 353 | As for Compound 304² 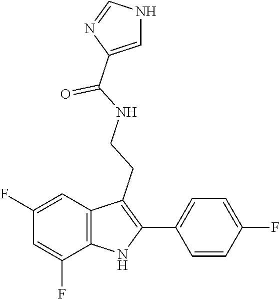 [SMILES: O=C(NCCc1c(-c2ccc(F)cc2)[nH]c2c(F)cc(F)cc12)c1c[nH]cn1] | 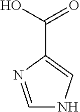 | ¹H NMR (300 MHz, Acetone-d₆) δ 10.79 (s, 1H), 9.18 (s, 1H), 8.55 (s, 1H), 8.14 (s, 1H), 7.75 (dd, J = 8.6, 5.3 Hz, 2H), 7.36-7.16 (m, 3H), 6.84 (ddd, J = 11.6, 9.7, 2.2 Hz, 1H), 3.70-3.62 (m, 2H), 3.17 (t, J = 7.4 Hz, 2H); LCMS m/z 385.13 [M + H]⁺. |
| 354 | As for Compound 304² 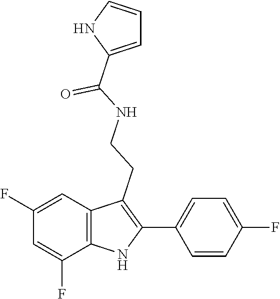 [SMILES: O=C(NCCc1c(-c2ccc(F)cc2)[nH]c2c(F)cc(F)cc12)c1ccc[nH]1] | 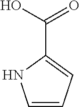 | ¹H NMR (300 MHz, Acetone-d₆) δ 10.68 (d, J = 30.3 Hz, 2H), 7.99 (s, 1H), 7.90-7.74 (m, 2H), 7.42- 7.32 (m, 1H), 7.31-7.18 (m, 2H), 6.96 (td, J = 2.7, 1.4 Hz, 1H), 6.84 (ddd, J = 11.1, 9.7, 2.2 Hz, 1H), 6.71-6.63 (m, 1H), 6.17-6.08 (m, 1H), 3.67-3.57 (m, 2H), 3.15- 3.04 (m, 2H); LCMS m/z 384.15 [M + H]⁺. |
| 355 | As for Compound 304² 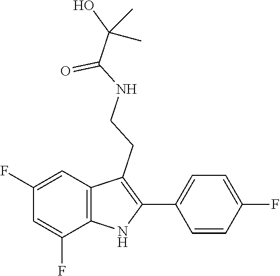 [SMILES: CC(C)(O)C(=O)NCCc1c(-c2ccc(F)cc2)[nH]c2c(F)cc(F)cc12] | 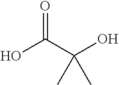 | ¹H NMR (300 MHz, Acetone-d₆) δ 7.89-7.79 (m, 2H), 7.38 (dd, J = 9.4, 2.2 Hz, 1H), 7.34-7.20 (m, 2H), 6.83 (ddd, J = 11.1, 9.7, 2.2 Hz, 1H), 3.64-3.46 (m, 2H), 3.16-2.97 (m, 2H), 1.33 (s, 6H); LCMS m/z 377.2 [M + H]⁺ |
| 356 | As for Compound 304 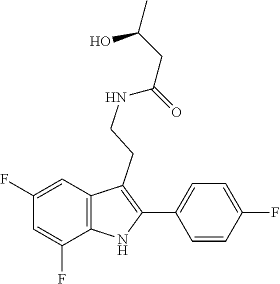 [SMILES: C[C@H](O)CC(=O)NCCc1c(-c2ccc(F)cc2)[nH]c2c(F)cc(F)cc12] | 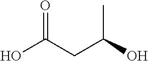 | LCMS m/z 377.2 [M + H]⁺ |
| 357 | As for Compound 304 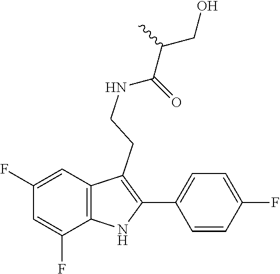 [SMILES: CC(CO)C(=O)NCCc1c(-c2ccc(F)cc2)[nH]c2c(F)cc(F)cc12] | 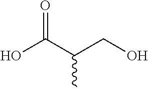 | ¹H NMR (300 MHz, Acetone-d₆) δ 7.99-7.70 (m, 2H), 7.54 (dd, J = 9.4, 2.2 Hz, 1H), 7.42-7.22 (m, 3H), 6.96-6.74 (m, 1H), 3.78- 3.41 (m, 3H), 3.23-2.93 (m, 2H), 2.46 (ddd, J = 12.1, 8.0, 6.1 Hz, 1H), 1.11 (dd, J = 33.0, 7.1 Hz, 3H); LCMS m/z 377.2 [M + H]⁺ |
| 358 | As for Compound 304² 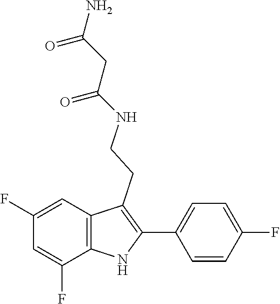 [SMILES: NC(=O)CC(=O)NCCc1c(-c2ccc(F)cc2)[nH]c2c(F)cc(F)cc12] | 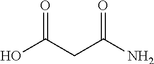 | ¹H NMR (300 MHz, Acetone-d₆) δ 7.93-7.61 (m, 3H), 7.44-7.18 (m, 3H), 6.84 (ddd, J = 11.5, 9.7, 2.2 Hz, 3H), 3.59 (q, J = 7.4, 6.9 Hz, 2H), 3.30 (s, 2H), 3.08 (dd, J = 9.0, 6.4 Hz, 2H); LCMS m/z 376.17 [M + H]⁺. |
| 359 | As for Compound 304² 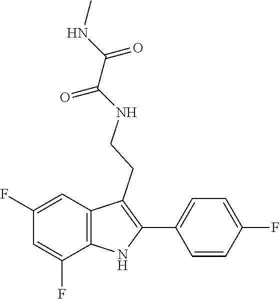 [SMILES: CNC(=O)C(=O)NCCc1c(-c2ccc(F)cc2)[nH]c2c(F)cc(F)cc12] | 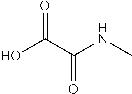 | ¹H NMR (300 MHz, Acetone-d₆) δ 7.91-7.35 (m, 2H), 7.54-7.29 (m, 1H), 7.35-7.12 (m, 2H), 6.84 (ddd, J = 11.1, 9.7, 2.2 Hz, 1H), 3.76-3.45 (m, 2H), 3.32-3.03 (m, 2H), 2.81 (s, 3H); LCMS m/z 376.17 [M + H]⁺. |
| 360 | As for Compound 304² 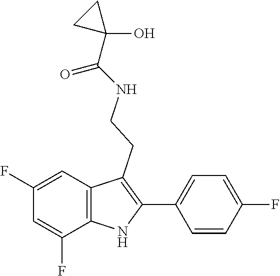 [SMILES: O=C(NCCc1c(-c2ccc(F)cc2)[nH]c2c(F)cc(F)cc12)C1(O)CC1] | 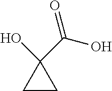 | ¹H NMR (300 MHz, Acetone-d₆) δ 8.09-7.68 (m, 2H), 7.38 (dd, J = 9.4, 2.2 Hz, 1H), 7.35-7.11 (m, 2H), 6.83 (ddd, J = 11.1, 9.6, 2.2 Hz, 1H), 3.73-3.41 (m, 2H), 3.21-2.99 (m, 2H), 1.28- 1.04 (m, 2H), 1.06-0.74 (m, 2H); LCMS m/z 375.16 [M + H]⁺. |
| 361 | As for Compound 304 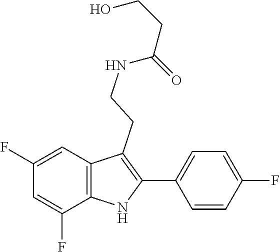 [SMILES: O=C(CCO)NCCc1c(-c2ccc(F)cc2)[nH]c2c(F)cc(F)cc12] | 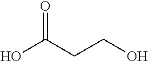 | ¹H NMR (300 MHz, Acetone-d₆) δ 10.75 (s, 1H), 7.89-7.74 (m, 2H), 7.60 (s, 1H), 7.35-7.23 (m, 3H), 6.83 (ddd, J = 11.1, 9.6, 2.2 Hz, 1H), 3.76 (t, J = 5.9 Hz, 2H), 3.59-3.46 (m, 2H), 3.10-2.99 (m, 2H), 2.37 (t, J = 5.9 Hz, 2H); LCMS m/z 363.2 [M + H]⁺ |
| 362 | As for Compound 304 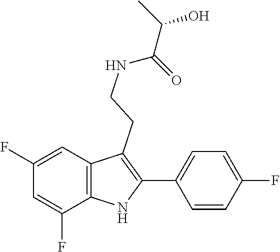 [SMILES: C[C@H](O)C(=O)NCCc1c(-c2ccc(F)cc2)[nH]c2c(F)cc(F)cc12] | 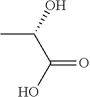 | ¹H NMR (300 MHz, Acetone-d₆) δ 7.99-7.74 (m, 2H), 7.44-7.14 (m, 3H), 6.84 (ddd, J = 11.1, 9.7, 2.2 Hz, 1H), 4.13 (q, J = 6.8 Hz, 1H), 3.74-3.38 (m, 2H), 3.23-3.03 (m, 2H), 1.29 (d, J = 6.8 Hz, 3H); LCMS m/z 363.2 [M + H]⁺ |
| 363 | As for Compound 306³ 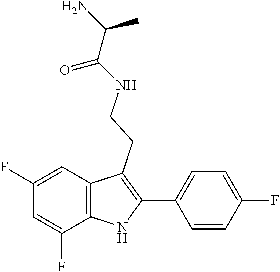 [SMILES: C[C@H](N)C(=O)NCCc1c(-c2ccc(F)cc2)[nH]c2c(F)cc(F)cc12] | 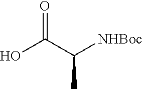 | ¹H NMR (300 MHz, Acetone-d₆) δ 7.84-7.73 (m, 2H), 7.35-7.23 (m, 3H), 6.85 (ddd, J = 11.3, 9.6, 2.2 Hz, 1H), 4.90 (q, J = 7.0 Hz, 1H), 3.58-3.47 (m, 2H), 3.30 (d, J = 0.8 Hz, 1H), 3.11-3.00 (m, 2H), 2.75- 2.47 (m, 1H), 1.57 (dd, J = 10.9, 7.0 Hz, 3H); LCMS m/z 362.1 [M + H]⁺ |
| 364 | As for Compound 304 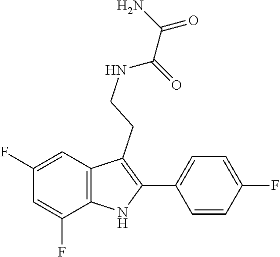 [SMILES: NC(=O)C(=O)NCCc1c(-c2ccc(F)cc2)[nH]c2c(F)cc(F)cc12] | 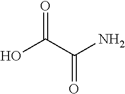 | ¹H NMR (300 MHz, Acetone-d₆) δ 10.77 (s, 1H), 8.36 (s, 1H), 7.87-7.74 (m, 2H), 7.66 (s, 1H), 7.43-7.24 (m, 3H), 7.01 (s, 1H), 6.84 (ddd, J = 11.0, 9.7, 2.2 Hz, 1H), 3.66-3.52 (m, 2H), 3.15-3.04 (m, 2H); LCMS m/z 362.1 [M + H]⁺ |
| 365 | As for Compound 304 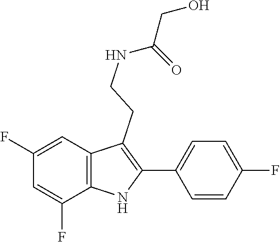 [SMILES: O=C(CO)NCCc1c(-c2ccc(F)cc2)[nH]c2c(F)cc(F)cc12] | 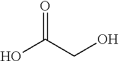 | ¹H NMR (300 MHz, Acetone-d₆) δ 10.76 (s, 1H), 7.88-7.75 (m, 2H), 7.40- 7.22 (m, 3H), 6.83 (ddd, J = 11.1, 9.6, 2.2 Hz, 1H), 3.98 (s, 2H), 3.66-3.52 (m, 2H), 3.13-3.02 (m, 2H); LCMS m/z 349.1 [M + H]⁺ |
| 366 | As for Compound 306³ 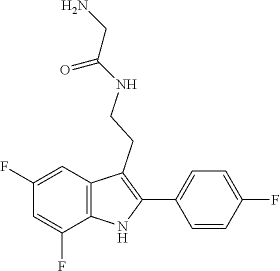 [SMILES: NCC(=O)NCCc1c(-c2ccc(F)cc2)[nH]c2c(F)cc(F)cc12] | 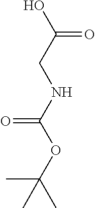 | ¹H NMR (300 MHz, Acetone-d₆) δ 7.83-7.72 (m, 2H), 7.35-7.23 (m, 3H), 6.84 (ddd, J = 11.1, 9.7, 2.2 Hz, 1H), 4.58 (s, 2H), 3.57 (dt, J = 15.4, 7.7 Hz, 2H), 3.06 (dd, J = 9.1, 6.4 Hz, 2H); LCMS m/z 348.1 [M + H]⁺ |
| 367 | As for Compound 304² 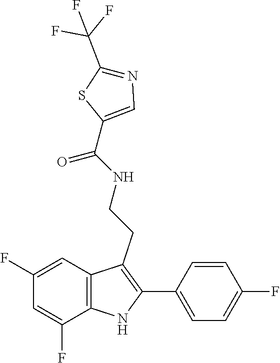 [SMILES: O=C(NCCc1c(-c2ccc(F)cc2)[nH]c2c(F)cc(F)cc12)c1cnc(C(F)(F)F)s1] | 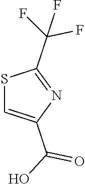 [SMILES: O=C(O)c1csc(C(F)(F)F)n1] | ¹H NMR (300 MHz, Acetone-d₆) δ 8.56 (s, 1H), 7.96-7.66 (m, 2H), 7.40 (dd, J = 9.4, 2.2 Hz, 1H), 7.29- 7.14 (m, 2H), 6.84 (ddd, J = 11.1, 9.6, 2.2 Hz, 1H), 3.97- 3.57 (m, 2H), 3.34-3.06 (m, 2H); LCMS m/z 470.15 [M + H]⁺. |
| 368 | As for Compound 304² 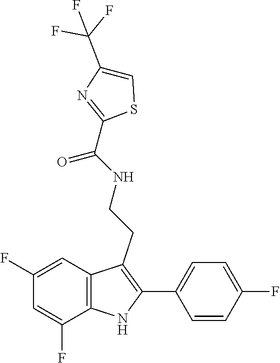 [SMILES: O=C(NCCc1c(-c2ccc(F)cc2)[nH]c2c(F)cc(F)cc12)c1nc(C(F)(F)F)cs1] | 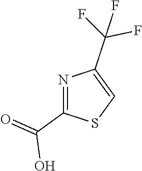 [SMILES: O=C(O)c1nc(C(F)(F)F)cs1] | ¹H NMR (300 MHz, Acetone-d₆) δ 8.63-8.31 (m, 2H), 7.91-7.66 (m, 2H), 7.44-7.35 (m, 1H), 7.31- 7.12 (m, 2H), 6.95-6.68 (m, 1H), 3.93-3.64 (m, 2H), 3.40-3.06 (m, 2H); LCMS m/z 470.12 [M + H]⁺. |
| 369 | As for Compound 304² 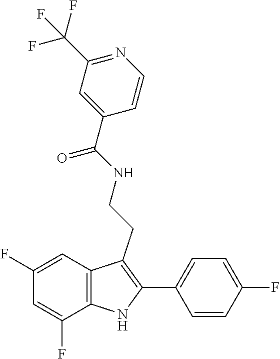 [SMILES: O=C(NCCc1c(-c2ccc(F)cc2)[nH]c2c(F)cc(F)cc12)c1ccnc(C(F)(F)F)c1] | 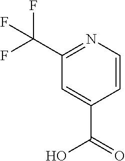 [SMILES: O=C(O)c1ccnc(C(F)(F)F)c1] | ¹H NMR (300 MHz, Acetone-d₆) δ 8.87 (dt, J = 5.0, 0.7 Hz, 1H), 8.08 (t, J = 1.1 Hz, 1H), 8.03-7.92 (m, 1H), 7.87-7.64 (m, 2H), 7.31 (dd, J = 9.4, 2.2 Hz, 1H), 7.27-6.99 (m, 2H), 6.85 (ddd, J = 11.1, 9.7, 2.2 Hz, 1H), 3.88-3.57 (m, 2H), 3.21 (t, J = 7.2 Hz, 2H); LCMS m/z 464.18 [M + H]⁺. |
| 370 | As for Compound 304 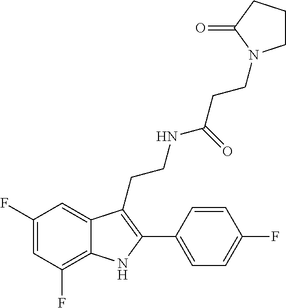 [SMILES: O=C(CCN1CCCC1=O)NCCc1c(-c2ccc(F)cc2)[nH]c2c(F)cc(F)cc12] | 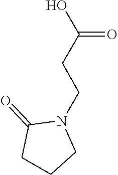 [SMILES: O=C(O)CCN1CCCC1=O] | LCMS m/z 430.2 [M + H]⁺. |
| 371 | As for Compound 304 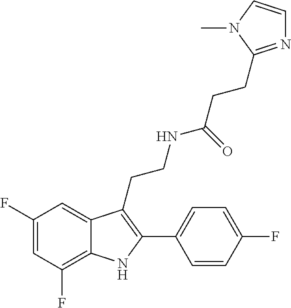 [SMILES: Cn1ccnc1CCC(=O)NCCc1c(-c2ccc(F)cc2)[nH]c2c(F)cc(F)cc12] | 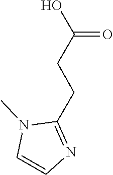 | LCMS m/z 427.2 [M + H]⁺. |
| 372 | As for Compound 304 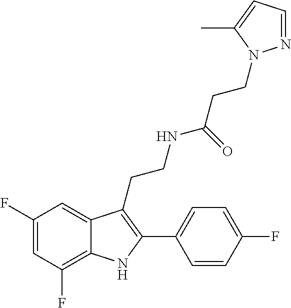 [SMILES: Cc1ccnn1CCC(=O)NCCc1c(-c2ccc(F)cc2)[nH]c2c(F)cc(F)cc12] | 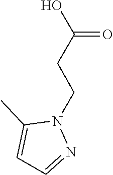 | LCMS m/z 427.2 [M + H]⁺. |
| 373 | As for Compound 304 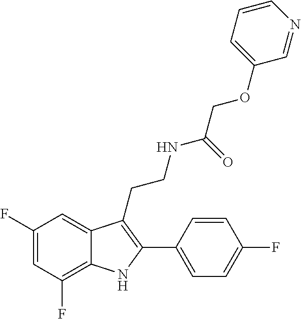 [SMILES: O=C(COc1cccnc1)NCCc1c(-c2ccc(F)cc2)[nH]c2c(F)cc(F)cc12] | 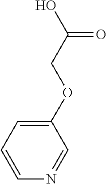 [SMILES: O=C(O)COc1cccnc1] | LCMS m/z 426.1 [M + H]⁺. |
| 374 | As for Compound 304 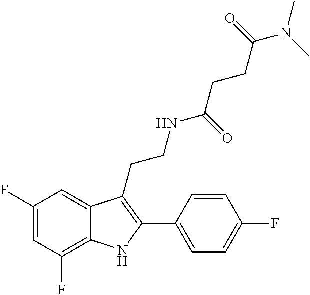 [SMILES: CN(C)C(=O)CCC(=O)NCCc1c(-c2ccc(F)cc2)[nH]c2c(F)cc(F)cc12] | 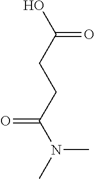 | LCMS m/z 418.2 [M + H]⁺. |
| 375 | As for Compound 304 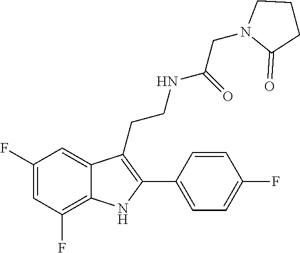 [SMILES: O=C(CN1CCCC1=O)NCCc1c(-c2ccc(F)cc2)[nH]c2c(F)cc(F)cc12] | 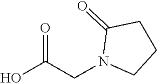 | LCMS m/z 416.2 [M + H]⁺. |
| 376 | As for Compound 304 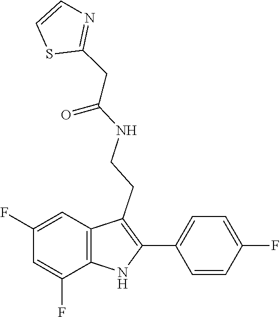 [SMILES: O=C(Cc1nccs1)NCCc1c(-c2ccc(F)cc2)[nH]c2c(F)cc(F)cc12] | 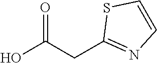 | ¹H NMR (300 MHz, Acetone-d₆) δ 10.76 (s, 1H), 8.01 (d, J = 3.6 Hz, 1H), 7.89 (s, 1H), 7.87-7.73 (m, 3H), 7.35-7.19 (m, 3H), 6.83 (ddd, J = 11.0, 9.7, 2.2 Hz, 1H), 4.25-4.02 (m, 2H), 3.63-3.47 (m, 2H), 3.13-3.00 (m, 2H); LCMS m/z 416.1 [M + H]⁺ |
| 377 | As for Compound 304² 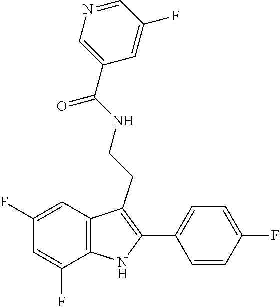 [SMILES: O=C(NCCc1c(-c2ccc(F)cc2)[nH]c2c(F)cc(F)cc12)c1cncc(F)c1] | 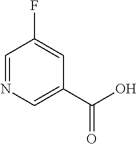 | ¹H NMR (300 MHz, Acetone-d₆) δ 8.82 (t, J = 1.7 Hz, 1H), 8.62 (d, J = 2.8 Hz, 1H), 7.89 (ddd, J = 9.4, 2.8, 1.8 Hz, 1H), 7.86-7.73 (m, 2H), 7.33 (dd, J = 9.4, 2.2 Hz, 1H), 7.28-7.12 (m, 2H), 6.85 (ddd, J = 11.1, 9.6, 2.2 Hz, 1H), 3.83-3.51 (m, 2H), 3.41-3.05 (m, 2H); LCMS m/z 414.12 [M + H]⁺. |
| 378 | As for Compound 304 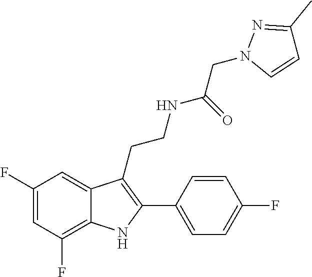 [SMILES: Cc1ccn(CC(=O)NCCc2c(-c3ccc(F)cc3)[nH]c3c(F)cc(F)cc23)n1] | 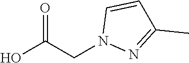 | LCMS m/z 413.1 [M + H]⁺. |
| 379 | As for Compound 304 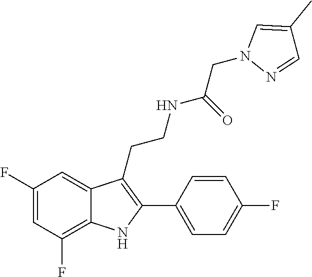 [SMILES: Cc1cnn(CC(=O)NCCc2c(-c3ccc(F)cc3)[nH]c3c(F)cc(F)cc23)c1] | 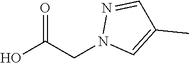 | LCMS m/z 413.2 [M + H]⁺. |
| 380 | As for Compound 304 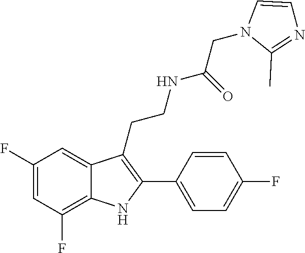 [SMILES: Cc1nccn1CC(=O)NCCc1c(-c2ccc(F)cc2)[nH]c2c(F)cc(F)cc12] | 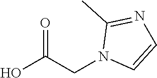 | LCMS m/z 413.0 [M + H]⁺. |
| 381 | As for Compound 304 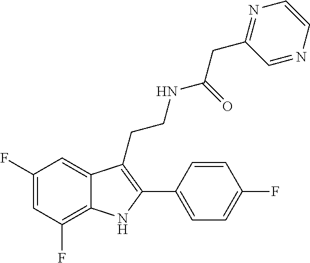 [SMILES: O=C(Cc1cnccn1)NCCc1c(-c2ccc(F)cc2)[nH]c2c(F)cc(F)cc12] | 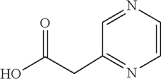 | LCMS m/z 413.0 [M + H]⁺. |
| 382 | As for Compound 304 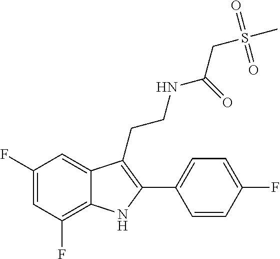 [SMILES: CS(=O)(=O)CC(=O)NCCc1c(-c2ccc(F)cc2)[nH]c2c(F)cc(F)cc12] | 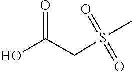 | LCMS m/z 413.0 [M + H]⁺. |
| 383 | As for Compound 304 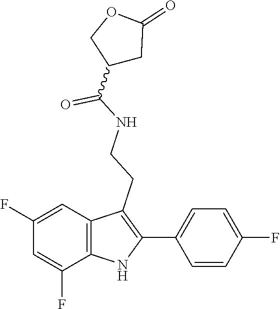 [SMILES: O=C1CC(C(=O)NCCc2c(-c3ccc(F)cc3)[nH]c3c(F)cc(F)cc23)CO1] | 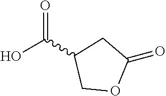 | ¹H NMR (300 MHz, Acetone-d₆) δ 7.95-7.66 (m, 2H), 7.44-7.15 (m, 3H), 6.84 (dddd, J = 11.1, 9.7, 2.2, 1.5 Hz, 1H), 4.38 (dd, J = 8.8, 8.2 Hz, 1H), 4.19 (dd, J = 8.8, 6.3 Hz, 1H), 3.66- 3.44 (m, 2H), 3.44-3.22 (m, 1H), 3.06 (td, J = 7.7, 7.2, 2.2 Hz, 2H), 2.89-2.44 (m, 2H); LCMS m/z 403.2 [M + H]⁺. |
| 384 | As for Compound 304² 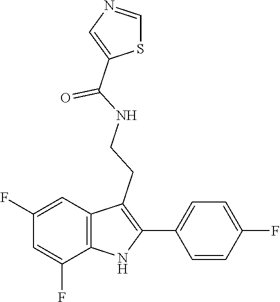 [SMILES: O=C(NCCc1c(-c2ccc(F)cc2)[nH]c2c(F)cc(F)cc12)c1cncs1] | 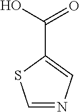 | ¹H NMR (300 MHz, Acetone-d₆) δ 9.22 (d, J = 0.7 Hz, 1H), 8.34 (d, J = 0.7 Hz, 1H), 7.89-7.68 (m, 2H), 7.31 (dd, J = 9.4, 2.2 Hz, 1H), 7.27-7.11 (m, 2H), 6.85 (ddd, J = 11.1, 9.6, 2.2 Hz, 1H), 3.80-3.50 (m, 2H), 3.28-2.92 (m, 2H); LCMS m/z 402.1 [M + H]⁺. |
| 385 | As for Compound 304² 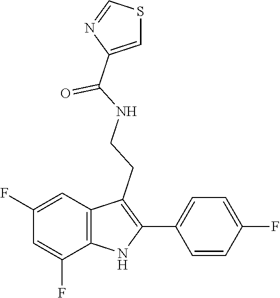 [SMILES: O=C(NCCc1c(-c2ccc(F)cc2)[nH]c2c(F)cc(F)cc12)c1cscn1] | 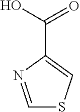 | ¹H NMR (300 MHz, Acetone-d₆) δ 9.04 (d, J = 2.1 Hz, 1H), 8.24 (d, J = 2.1 Hz, 2H), 7.99-7.74 (m, 2H), 7.42 (dd, J = 9.4, 2.2 Hz, 1H), 7.34-7.16 (m, 2H), 6.84 (ddd, J = 11.1, 9.7, 2.2 Hz, 1H), 3.92-3.57 (m, 2H), 3.40-2.97 (m, 2H); LCMS m/z 402.1 [M + H]⁺. |
| 386 | As for Compound 304 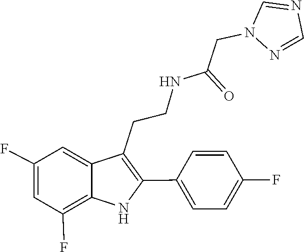 [SMILES: O=C(Cn1cncn1)NCCc1c(-c2ccc(F)cc2)[nH]c2c(F)cc(F)cc12] | 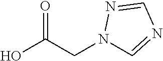 | LCMS m/z 400.2 [M + H]⁺. |
| 387 | As for Compound 304 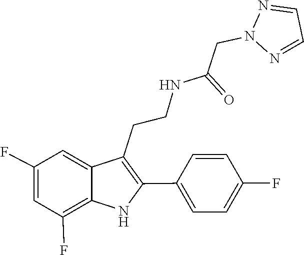 [SMILES: O=C(Cn1nccn1)NCCc1c(-c2ccc(F)cc2)[nH]c2c(F)cc(F)cc12] | 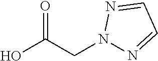 | LCMS m/z 400.2 [M + H]⁺. |
| 388 | As for Compound 304 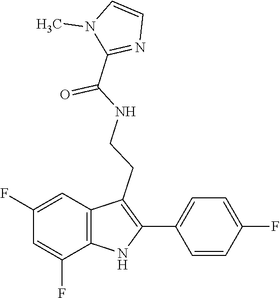 [SMILES: Cn1ccnc1C(=O)NCCc1c(-c2ccc(F)cc2)[nH]c2c(F)cc(F)cc12] | 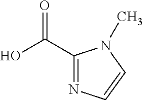 | LCMS m/z 399.2 [M + H]⁺. |
| 389 | As for Compound 304 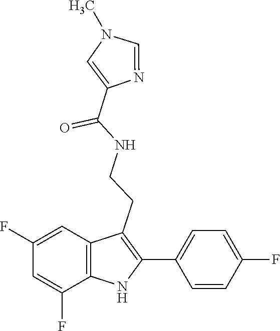 [SMILES: Cn1cnc(C(=O)NCCc2c(-c3ccc(F)cc3)[nH]c3c(F)cc(F)cc23)c1] | 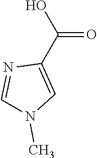 | LCMS m/z 399.2 [M + H]⁺. |
| 390 | As for Compound 304 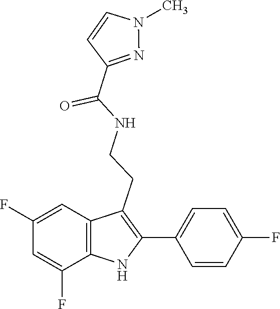 [SMILES: Cn1ccc(C(=O)NCCc2c(-c3ccc(F)cc3)[nH]c3c(F)cc(F)cc23)n1] | 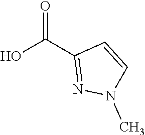 | LCMS m/z 399.2 [M + H]⁺. |
| 391 | As for Compound 304 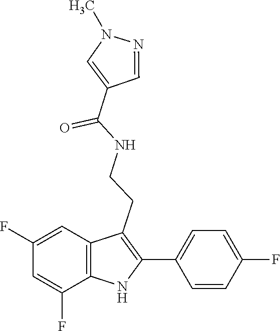 [SMILES: Cn1cc(C(=O)NCCc2c(-c3ccc(F)cc3)[nH]c3c(F)cc(F)cc23)cn1] | 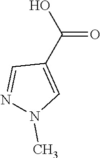 | LCMS m/z 399.2 [M + H]⁺. |
| 392 | As for Compound 304 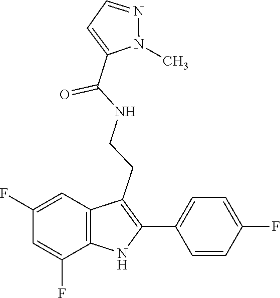 [SMILES: Cn1nccc1C(=O)NCCc1c(-c2ccc(F)cc2)[nH]c2c(F)cc(F)cc12] | 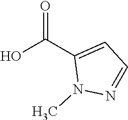 | LCMS m/z 399.2 [M + H]⁺. |
| 393 | As for Compound 304 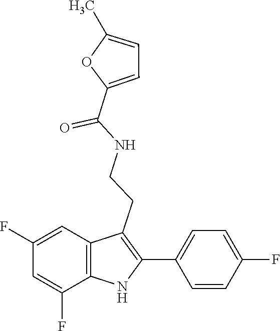 [SMILES: Cc1ccc(C(=O)NCCc2c(-c3ccc(F)cc3)[nH]c3c(F)cc(F)cc23)o1] | 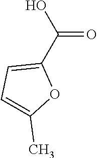 | LCMS m/z 399.1 [M + H]⁺. |
| 394 | As for Compound 304 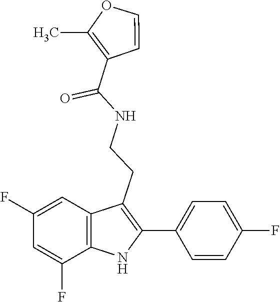 [SMILES: Cc1occc1C(=O)NCCc1c(-c2ccc(F)cc2)[nH]c2c(F)cc(F)cc12] | 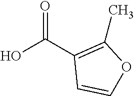 | LCMS m/z 399.1 [M + H]⁺. |
| 395 | As for Compound 304 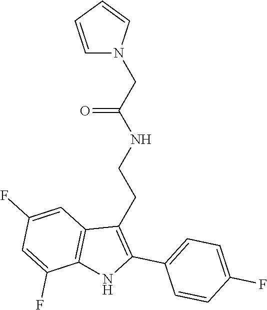 [SMILES: O=C(Cn1cccc1)NCCc1c(-c2ccc(F)cc2)[nH]c2c(F)cc(F)cc12] | 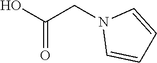 | LCMS m/z 398.2 [M + H]⁺. |
| 396 | As for Compound 304 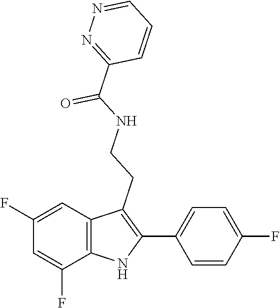 [SMILES: O=C(NCCc1c(-c2ccc(F)cc2)[nH]c2c(F)cc(F)cc12)c1cccnn1] | 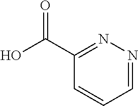 | LCMS m/z 397.2 [M + H]⁺. |
| 397 | As for Compound 304 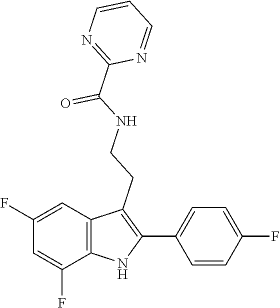 [SMILES: O=C(NCCc1c(-c2ccc(F)cc2)[nH]c2c(F)cc(F)cc12)c1ncccn1] | 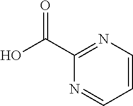 | LCMS m/z 397.2 [M + H]⁺. |
| 398 | As for Compound 304 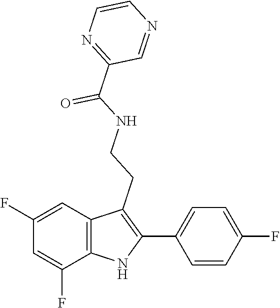 [SMILES: O=C(NCCc1c(-c2ccc(F)cc2)[nH]c2c(F)cc(F)cc12)c1cnccn1] | 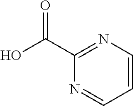 | LCMS m/z 397.2 [M + H]⁺. |
| 399 | As for Compound 304 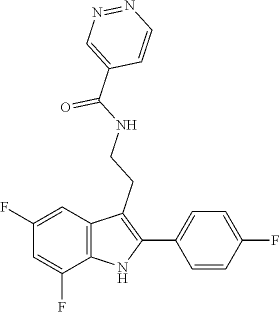 [SMILES: O=C(NCCc1c(-c2ccc(F)cc2)[nH]c2c(F)cc(F)cc12)c1ccnnc1] | 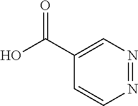 | LCMS m/z 397.2 [M + H]⁺. |
| 400 | As for Compound 304 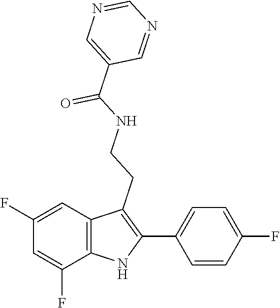 [SMILES: O=C(NCCc1c(-c2ccc(F)cc2)[nH]c2c(F)cc(F)cc12)c1cncnc1] | 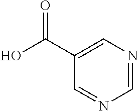 | LCMS m/z 397.1 [M + H]⁺. |
| 401 | As for Compound 304² 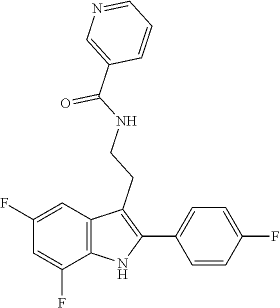 [SMILES: O=C(NCCc1c(-c2ccc(F)cc2)[nH]c2c(F)cc(F)cc12)c1cccnc1] | 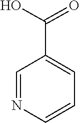 | ¹H NMR (300 MHz, Acetone-d₆) δ 9.30 (d, J = 2.0 Hz, 1H), 8.85 (ddd, J = 8.1, 2.1, 1.4 Hz, 1H), 8.19 (ddd, J = 8.1, 5.6, 0.7 Hz, 1H), 8.00 (s, 1H), 7.89-7.67 (m, 2H), 7.32 (dd, J = 9.4, 2.2 Hz, 1H), 7.27-7.15 (m, 2H), 6.86 (ddd, J = 11.1, 9.7, 2.2 Hz, 1H), 3.88-3.64 (m, 2H), 3.22 (dd, J = 8.0, 6.6 Hz, 2H); LCMS m/z 396.14 [M + H]⁺. |
| 402 | As for Compound 304 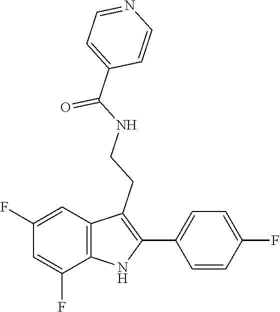 [SMILES: O=C(NCCc1c(-c2ccc(F)cc2)[nH]c2c(F)cc(F)cc12)c1ccncc1] | 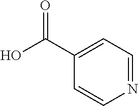 | LCMS m/z 396.1 [M + H]⁺. |
| 403 | As for Compound 304 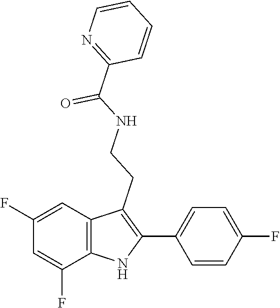 [SMILES: O=C(NCCc1c(-c2ccc(F)cc2)[nH]c2c(F)cc(F)cc12)c1ccccn1] | 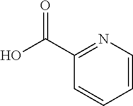 | LCMS m/z 396.1 [M + H]⁺. |
| 404 | As for Compound 304 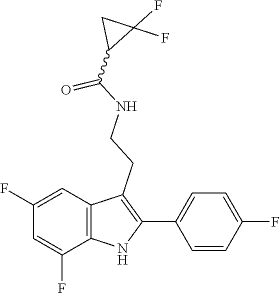 [SMILES: O=C(NCCc1c(-c2ccc(F)cc2)[nH]c2c(F)cc(F)cc12)C1CC1(F)F] | 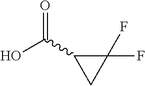 | LCMS m/z 395.1 [M + H]⁺. |
| 405 | As for Compound 304 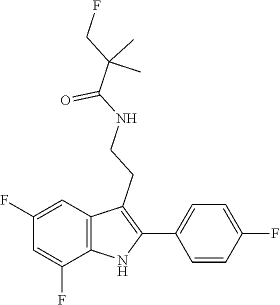 [SMILES: CC(C)(CF)C(=O)NCCc1c(-c2ccc(F)cc2)[nH]c2c(F)cc(F)cc12] | 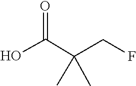 | LCMS m/z 393.2 [M + H]⁺. |
| 406 | As for Compound 304 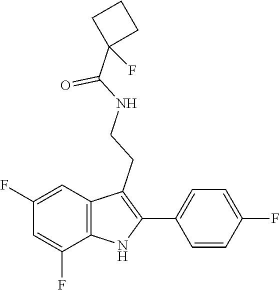 [SMILES: O=C(NCCc1c(-c2ccc(F)cc2)[nH]c2c(F)cc(F)cc12)C1(F)CCC1] | 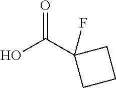 | LCMS m/z 391.2 [M + H]⁺. |
| 407 | As for Compound 304 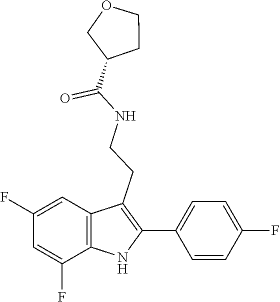 [SMILES: O=C(NCCc1c(-c2ccc(F)cc2)[nH]c2c(F)cc(F)cc12)[C@H]1CCOC1] | 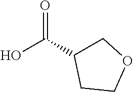 | LCMS m/z 389.2 [M + H]⁺. |
| 408 | As for Compound 304 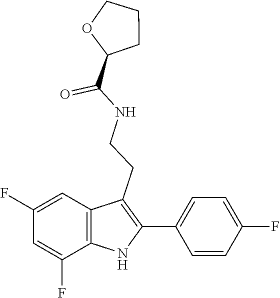 [SMILES: O=C(NCCc1c(-c2ccc(F)cc2)[nH]c2c(F)cc(F)cc12)[C@@H]1CCCO1] | 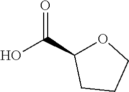 | LCMS m/z 389.2 [M + H]⁺. |
| 409 | As for Compound 304 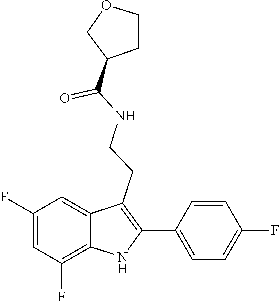 [SMILES: O=C(NCCc1c(-c2ccc(F)cc2)[nH]c2c(F)cc(F)cc12)[C@@H]1CCOC1] | 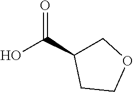 | LCMS m/z 389.2 [M + H]⁺. |
| 410 | As for Compound 304 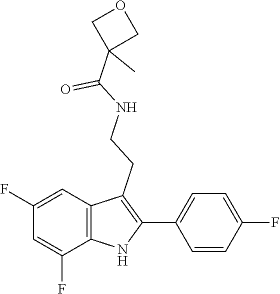 [SMILES: CC1(C(=O)NCCc2c(-c3ccc(F)cc3)[nH]c3c(F)cc(F)cc23)COC1] | 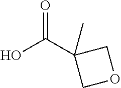 | LCMS m/z 389.2 [M + H]⁺. |
| 411 | As for Compound 304² 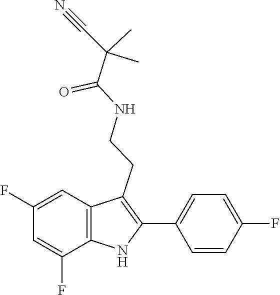 [SMILES: CC(C)(C#N)C(=O)NCCc1c(-c2ccc(F)cc2)[nH]c2c(F)cc(F)cc12] | 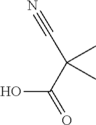 | ¹H NMR (300 MHz, Acetone-d₆) δ 7.94-7.70 (m, 2H), 7.44-7.15 (m, 3H), 6.84 (ddd, J = 11.1, 9.7, 2.2 Hz, 1H), 3.80-3.44 (m, 2H), 3.29-2.86 (m, 2H), 1.50 (s, 6H); LCMS m/z 386.2 [M + H]⁺. |
| 412 | As for Compound 304 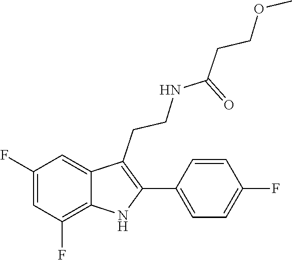 [SMILES: COCCC(=O)NCCc1c(-c2ccc(F)cc2)[nH]c2c(F)cc(F)cc12] | 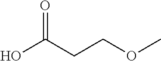 | LCMS m/z 377.2 [M + H]+. |
| 413 | As for Compound 304² 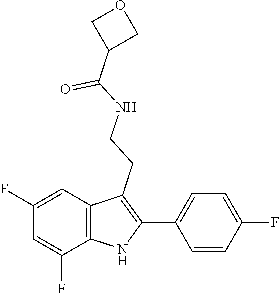 [SMILES: O=C(NCCc1c(-c2ccc(F)cc2)[nH]c2c(F)cc(F)cc12)C1COC1] | 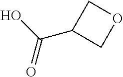 | ¹H NMR (300 MHz, Acetone-d₆) δ 10.74 (s, 1H), 7.86-7.61 (m, 2H), 7.40- 7.13 (m, 3H), 6.83 (ddd, J = 11.1, 9.6, 2.2 Hz, 1H), 4.70- 4.52 (m, 4H), 3.78-3.64 (m, 1H), 3.57-3.44 (m, 2H), 3.09-2.98 (m, 2H); LCMS m/z 375.16 [M + H]⁺. |
| 414 | As for Compound 307¹ 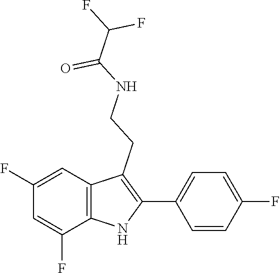 [SMILES: O=C(NCCc1c(-c2ccc(F)cc2)[nH]c2c(F)cc(F)cc12)C(F)F] | 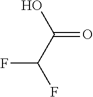 | ¹H NMR (300 MHz, Acetone-d₆) δ 7.93-7.64 (m, 2H), 7.30 (ddd, J = 8.8, 5.4, 2.9 Hz, 3H), 6.85 (ddd, J = 11.1, 9.6, 2.2 Hz, 1H), 6.06 (t, J = 54.1 Hz, 1H), 3.76- 3.48 (m, 2H), 3.28-2.88 (m, 2H); LCMS m/z 369.13 [M + H]⁺. |
| 415 | As for Compound 304 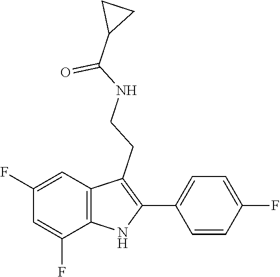 [SMILES: O=C(NCCc1c(-c2ccc(F)cc2)[nH]c2c(F)cc(F)cc12)C1CC1] | 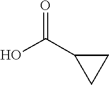 | LCMS m/z 359.2 [M + H]⁺. |
| 416 | As for Compound 304 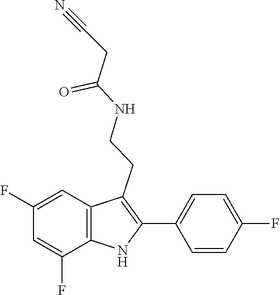 [SMILES: N#CCC(=O)NCCc1c(-c2ccc(F)cc2)[nH]c2c(F)cc(F)cc12] | 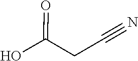 | ¹H NMR (300 MHz, Acetone-d₆) δ 7.90-7.73 (m, 2H), 7.41-7.15 (m, 3H), 6.84 (ddd, J = 11.0, 9.6, 2.2 Hz, 1H), 3.67-3.32 (m, 4H), 3.23-2.91 (m, 2H); LCMS m/z 358.1 [M + H]⁺. |
| 417 | As for Compound 304 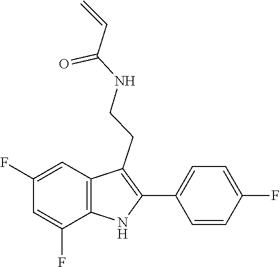 [SMILES: C=CC(=O)NCCc1c(-c2ccc(F)cc2)[nH]c2c(F)cc(F)cc12] | 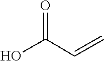 | ¹H NMR (300 MHz, Acetone-d₆) δ 7.94-7.68 (m, 2H), 7.41-7.19 (m, 3H), 6.84 (ddd, J = 11.1, 9.6, 2.2 Hz, 1H), 6.35-6.06 (m, 2H), 5.56 (dd, J = 7.8, 4.5 Hz, 1H), 3.70-3.36 (m, 2H), 3.25-2.82 (m, 2H); LCMS m/z 345.1 [M + H]⁺. |

¹Boc deprotection was performed after amide formation.
²Purification by reversed-phase HPLC. Method: C18 Waters Sunfire column (30 × 150 mm, 5 micron). Gradient: MeCN in H₂O with 0.1% trifluoroacetic acid.
³Step 3. (triphosgene and DIPEA treatment) was omitted.

### Compound 418

### N-[2-[5,7-difluoro-2-(4-fluorophenyl)-1H-indol-3-yl]ethyl]acetamide (418)

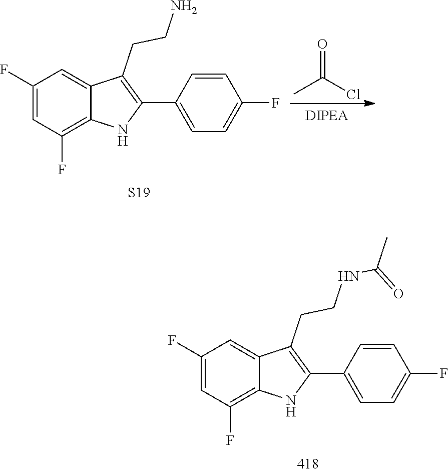

### Preparation of N-[2-[5,7-difluoro-2-(4-fluorophenyl)-1H-indol-3-yl]ethyl]acetamide (418)

A solution of 2-[5,7-difluoro-2-(4-fluorophenyl)-1H-indol-3-yl]ethanamine S19 (56 mg, 0.1929 mmol) in dichloromethane (2 mL) was treated with acetyl chloride (17 μL, 0.2391 mmol) followed by DIPEA (51 μL, 0.2928 mmol). The resulting solution was stirred at ambient temperature overnight then partitioned between dichloromethane and 1 M NaOH. The organic layer was concentrated in vacuo then purified by silica gel chromatography (Gradient: 0-20% EtOAc in heptane) to afford N-[2-[5,7-difluoro-2-(4-fluorophenyl)-1H-indol-3-yl]ethyl]acetamide (32.9 mg, 49%). ¹H NMR (300 MHz, Chloroform-d) δ 8.19 (s, 1H), 7.60-7.53 (m, 2H), 7.24-7.17 (m, 2H), 7.10 (dd, J=9.0, 2.2 Hz, 1H), 6.76 (ddd, J=10.7, 9.4, 2.1 Hz, 1H), 5.47 (s, 1H), 3.49 (q, J=6.9 Hz, 2H), 3.02 (t, J=7.1 Hz, 2H), 1.85 (s, 3H). LCMS m/z 333.15 [M+H]⁺

### Compound 419

### N-[2-[5,7-difluoro-2-(4-fluorophenyl)-1H-indol-3-yl]ethyl]formamide (419)

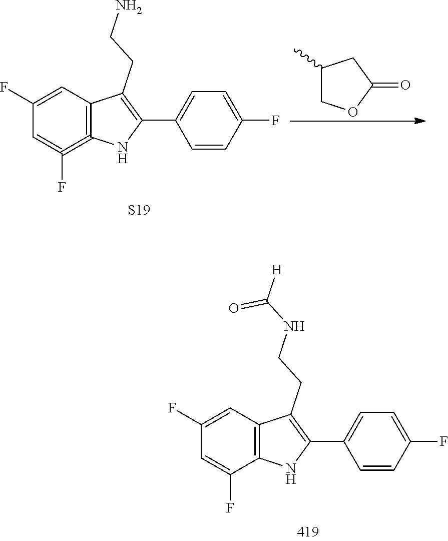

### Preparation of N-[2-[5,7-difluoro-2-(4-fluorophenyl)-1H-indol-3-yl]ethyl]formamide (419)

To a solution of 2-[5,7-difluoro-2-(4-fluorophenyl)-1H-indol-3-yl]ethanamine (Trifluoroacetate salt) S19 (25 mg, 0.0546 mmol) was dissolved in dimethylformamide (2 mL). To the reaction was added 4-methyltetrahydrofuran-2-one (10.9 mg, 0.1092 mmol) and warmed to 110° C. overnight. The mixture was then purified by reversed-phase HPLC (Method: C18 Waters Sunfire column (30×150 mm, 5 micron). Gradient: MeCN in H₂O with 0.1% trifluoroacetic acid) to afford the product N-[2-[5,7-difluoro-2-(4-fluorophenyl)-1H-indol-3-yl]ethyl]formamide (5.8 mg, 14%). ¹H NMR (300 MHz, Acetone-d₆) δ 8.14 (d, J=1.5 Hz, 1H), 7.96-7.66 (m, 2H), 7.41-7.15 (m, 3H), 6.92-6.73 (m, 1H), 3.72-3.46 (m, 2H), 3.25-2.95 (m, 2H). LCMS m/z 319.06 [M+H]⁺.

### Compound 420

### N-(2-(5,7-difluoro-2-(4-fluorophenyl)-1H-indol-3-yl)ethyl)-2-(2,5-dioxoimidazolidin-4-yl)ethane-1-sulfonamide (420)

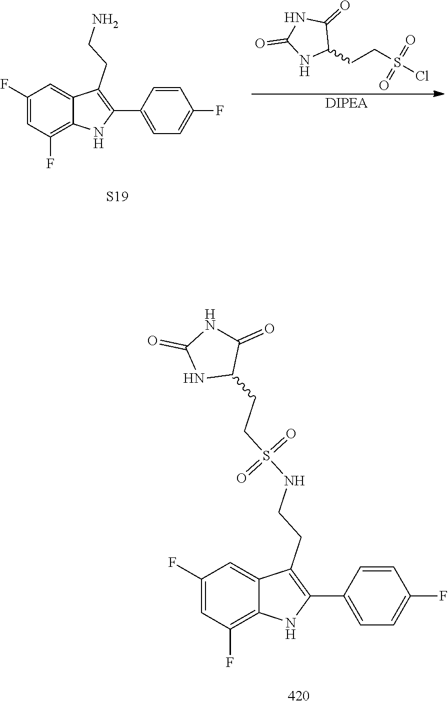 [SMILES: NCCc1c(-c2ccc(F)cc2)[nH]c2c(F)cc(F)cc12 ; O=C1NC(=O)C(CCS(=O)(=O)Cl)N1 ; "S19" ; O=C1NC(=O)C(CCS(=O)(=O)NCCc2c(-c3ccc(F)cc3)[nH]c3c(F)cc(F)cc23)N1]

### Preparation of N-(2-(5,7-difluoro-2-(4-fluorophenyl)-1H-indol-3-yl)ethyl)-2-(2,5-dioxoimidazolidin-4-yl)ethane-1-sulfonamide (420)

2-[5,7-difluoro-2-(4-fluorophenyl)-1H-indol-3-yl]ethanamine (160 mg, 0.4868 mmol) S19 was dissolved in DMF (16 mL). To this solution was added DIPEA (200 μL, 1.148 mmol) and the mixture was split into 8 equal portions. To one portion was added 2-(2,5-dioxoimidazolidin-4-yl)ethanesulfonyl chloride (50 mg, 0.2206 mmol). The reaction was stirred overnight. The crude mixture was purified by reversed-phase HPLC (Method: C18 Waters Sunfire column (30×150 mm, 5 micron). Gradient: MeCN in H₂O with 0.1% trifluoroacetic acid) to afford N-[2-[5,7-difluoro-2-(4-fluorophenyl)-1H-indol-3-yl]ethyl]-2-(2,5-dioxoimidazolidin-4-yl)ethanesulfonamide (10.0 mg, 20%). ¹H NMR (300 MHz, Acetone-d₆) δ 7.94-7.67 (m, 2H), 7.59-7.19 (m, 3H), 7.07 (s, 1H), 6.84 (ddd, J=11.1, 9.7, 2.2 Hz, 1H), 6.42 (t, J=6.3 Hz, 1H), 4.28 (ddd, J=7.2, 5.4, 1.5 Hz, 1H), 3.55-3.35 (m, 2H), 3.35-3.03 (m, 4H), 2.38-2.14 (m, 1H). LCMS m/z 481.19 [M+H]⁺.

### Compound 421

### 1-((2-(5,7-difluoro-2-(4-fluorophenyl)-1H-indol-3-yl)ethyl)amino)propan-2-ol (421)

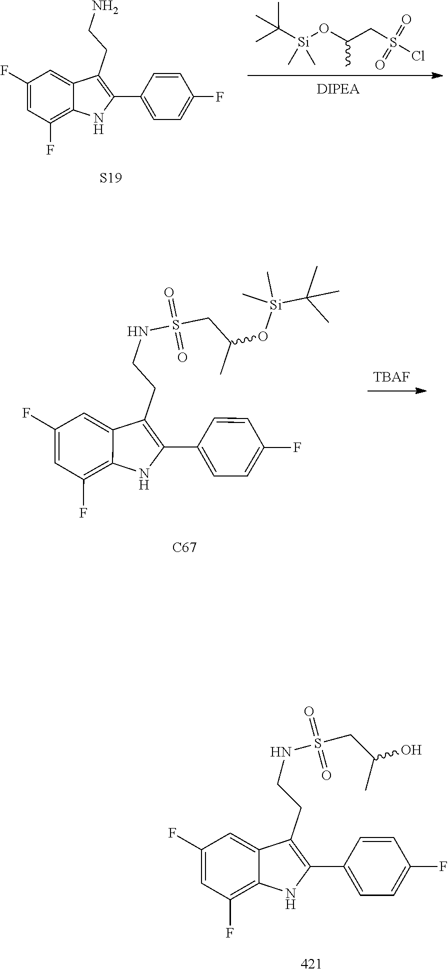

### Step 1. Synthesis of 2-((tert-butyldimethylsilyl)oxy)-N-(2-(5,7-difluoro-2-(4-fluorophenyl)-1H-indol-3-yl)ethyl)propane-1-sulfonamide (C67)

2-[5,7-difluoro-2-(4-fluorophenyl)-1H-indol-3-yl]ethanamine S19 (80 mg, 0.2434 mmol) was dissolved in DMF (8 mL), DIPEA (50 μL, 0.2871 mmol) was added, and the mixture was divided into 4 equal portions. To one portion was added, 2-[tert-butyl(dimethyl)silyl]oxypropane-1-sulfonyl chloride (70 mg, 0.2565 mmol). The reaction was stirred at room temperature overnight. The crude mixture was purified by reversed-phase HPLC (Method: C18 Waters Sunfire column (30×150 mm, 5 micron). Gradient: MeCN in H₂O with 0.1% TFA) to afford 2-[tert-butyl(dimethyl)silyl]oxy-N-[2-[5,7-difluoro-2-(4-fluorophenyl)-1H-indol-3-yl]ethyl]propane-1-sulfonamide (15 mg, 17%). LCMS m/z 527.21 [M+H]⁺.

### Step 2. Synthesis of 1-((2-(5,7-difluoro-2-(4-fluorophenyl)-1H-indol-3-yl)ethyl)amino)propan-2-ol (421)

2-((tert-butyldimethylsilyl)oxy)-N-(2-(5,7-difluoro-2-(4-fluorophenyl)-1H-indol-3-yl)ethyl)propane-1-sulfonamide C67 (20 mg, 0.03797 mmol) was dissolved in THF (5 mL). To this solution was added TBAF (100 μL of 1M, 0.1000 mmol) and the reaction was stirred overnight. The mixture was concentrated and re-dissolved in DMSO (2 mL). The crude solution was purified by reversed-phase HPLC (Method: C18 Waters Sunfire column (30×150 mm, 5 micron). Gradient: MeCN in H₂O with 0.1% trifluoroacetic acid) to afford 1-((2-(5,7-difluoro-2-(4-fluorophenyl)-1H-indol-3-yl)ethyl)amino)propan-2-ol (3.2 mg, 17%). ¹H NMR (300 MHz, Acetone-d₆) δ 7.96-7.63 (m, 2H), 7.46-7.14 (m, 3H), 6.85 (ddd, J=11.1, 9.7, 2.2 Hz, 1H), 4.29-4.12 (m, 2H), 3.55-3.29 (m, 3H), 3.29-2.97 (m, 4H), 1.22 (d, J=6.3 Hz, 3H). LCMS m/z 413.08 [M+H]⁺.

### Compounds 422-425

Compounds 422-425 (see Table 12) were prepared from intermediate S19 using the appropriate reagents, using the sulfonamide formation method as described for compound 421. Sulfonyl chloride reagents were obtained from commercial sources. Any modifications to methods are noted in Table 12 and accompanying footnotes.

TABLE 12
Method of preparation, structure, physicochemical data for compounds 422-425

| | | Sulfonyl | |
|---|---|---|---|
| | | Chloride | ¹H NMR; LCMS m/z |
| Compound | Product | Reagent | [M + H]⁺ |
| 422 | 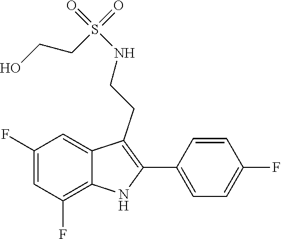 [SMILES: O=S(=O)(CCO)NCCc1c(-c2ccc(F)cc2)[nH]c2c(F)cc(F)cc12] | 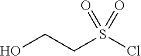 | ¹H NMR (300 MHz, Acetone-d₆) δ 7.79 (ddd, J = 8.8, 5.2, 2.6 Hz, 2H), 7.52- 7.14 (m, 2H), 6.99-6.72 (m, 1H), 3.94 (t, J = 6.3 Hz, 2H), 3.40 (d, J = 8.6 Hz, 1H), 3.33 (s, 5H), 3.20-2.95 (m, 2H); LCMS m/z 399.13 [M + H]⁺ |
| 423 | 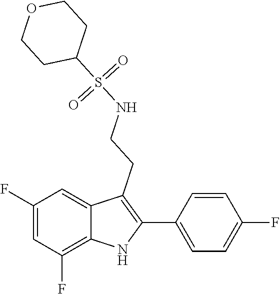 [SMILES: O=S(=O)(NCCc1c(-c2ccc(F)cc2)[nH]c2c(F)cc(F)cc12)C1CCOCC1] | 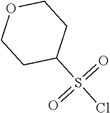 | ¹H NMR (300 MHz, Acetone-d₆) δ 7.89-7.63 (m, 2H), 7.40-7.16 (m, 3H), 6.85 (ddd, J = 11.6, 9.7, 2.2 Hz, 1H), 4.12-3.82 (m, 2H), 3.53-3.38 (m, 2H), 3.28 (td, J = 11.9, 2.1 Hz, 2H), 3.22- 2.93 (m, 3H), 1.95-1.78 (m, 2H), 1.66 (qd, J = 12.3, 4.7 Hz, 2H); LCMS m/z 439.13 [M + H]⁺ |
| 424 | 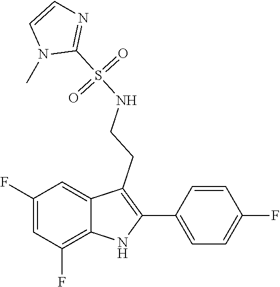 [SMILES: Cn1ccnc1S(=O)(=O)NCCc1c(-c2ccc(F)cc2)[nH]c2c(F)cc(F)cc12] | 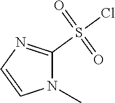 | ¹H NMR (300 MHz, Acetone-d₆) δ 10.77 (s, 1H), 7.85-7.71 (m, 2H), 7.35- 7.19 (m, 4H), 6.99 (d, J = 1.1 Hz, 1H), 6.83 (ddd, J = 11.0, 9.7, 2.2 Hz, 1H), 3.90 (s, 3H), 3.58-3.42 (m, 3H), 3.20-2.99 (m, 2H); LCMS m/z 435.06 [M + H]⁺ |
| 425 | 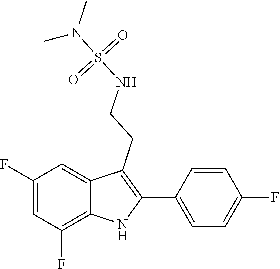 [SMILES: CN(C)S(=O)(=O)NCCc1c(-c2ccc(F)cc2)[nH]c2c(F)cc(F)cc12] | 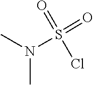 | ¹H NMR (300 MHz, Acetone-d₆) δ 8.00-7.61 (m, 2H), 7.44-7.16 (m, 3H), 6.85 (ddd, J = 11.1, 9.7, 2.2 Hz, 1H), 3.54-3.34 (m, 2H), 3.14 (dd, J = 9.5, 6.5 Hz, 2H), 2.83 (s, 6H); LCMS m/z 397.96 [M + H]⁺ |

### Compound 426

### 1-[2-[5,7-difluoro-2-(4-fluorophenyl)-1H-indol-3-yl]ethylcarbamoyloxymethyl]cyclopropanecarboxylic acid (426)

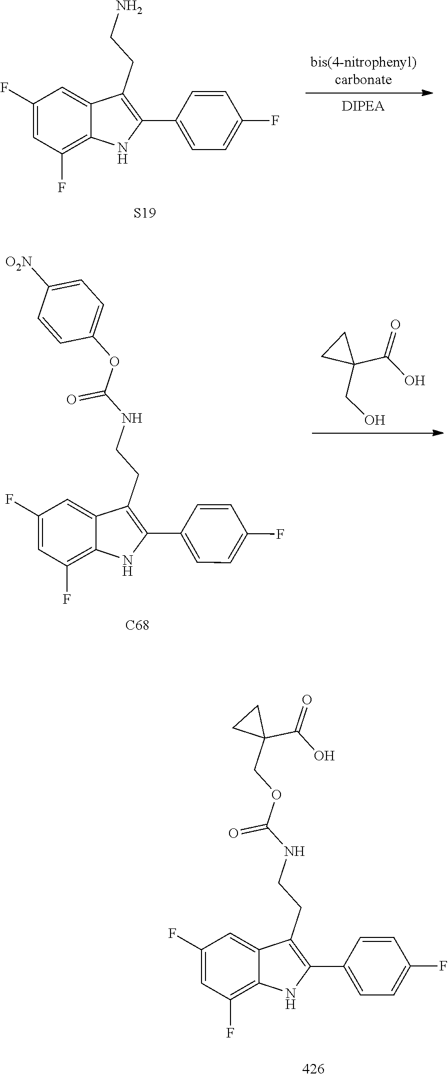 [SMILES: NCCc1c(-c2ccc(F)cc2)[nH]c2c(F)cc(F)cc12 ; "S19" ; O=C(NCCc1c(-c2ccc(F)cc2)[nH]c2c(F)cc(F)cc12)Oc1ccc([N+](=O)[O-])cc1 ; O=C(O)C1(CO)CC1 ; "C68" ; O=C(NCCc1c(-c2ccc(F)cc2)[nH]c2c(F)cc(F)cc12)OCC1(C(=O)O)CC1]

### Step 1. Synthesis of (4-nitrophenyl) N-[2-[5,7-difluoro-2-(4-fluorophenyl)-1H-indol-3-yl]ethyl]carbamate (C68)

To a solution of bis(4-nitrophenyl) carbonate (800 mg, 2.630 mmol) dissolved in DMF (20 mL) was added DIPEA (800 μL, 4.593 mmol) and 2-[5,7-difluoro-2-(4-fluorophenyl)-1H-indol-3-yl]ethanamine S19 (750 mg, 2.282 mmol). The solution was stirred at room temperature for 30 min, and then used as is in subsequent steps without further purification (4-nitrophenyl) N-[2-[5,7-difluoro-2-(4-fluorophenyl)-1H-indol-3-yl]ethyl]carbamate. LCMS m/z 456.1 [M+H]⁺.

### Step 2. Synthesis of 1-[2-[5,7-difluoro-2-(4-fluorophenyl)-1H-indol-3-yl]ethylcarbamoyloxy-methyl]cyclopropanecarboxylic acid (426)

To a solution of (4-nitrophenyl) N-[2-[5,7-difluoro-2-(4-fluorophenyl)-1H-indol-3-yl]ethyl]carbamate C67 (20 mg, 0.044 mmol) dissolved in DMF (4 mL) was added 1-(hydroxymethyl)cyclopropanecarboxylic acid (20 mg, 0.1722 mmol) and the solution was stirred overnight at room temperature. The crude mixture was purified by reversed-phase HPLC (Method: C18 Waters Sunfire column (30×150 mm, 5 micron). Gradient: MeCN in H₂O with 0.1% trifluoroacetic acid) to yield 1-[2-[5,7-difluoro-2-(4-fluorophenyl)-1H-indol-3-yl]ethylcarba-moyloxymethyl]cyclopropanecarboxylic acid (4.2 mg, 23%). ¹H NMR (300 MHz, Acetone-d₆) δ 7.93-7.72 (m, 2H), 7.41-7.19 (m, 2H), 6.82 (ddd, J=11.5, 9.7, 2.2 Hz, 1H), 3.57 (t, J=5.8 Hz, 1H), 3.46 (t, J=7.4 Hz, 2H), 3.01 (dd, J=8.7, 6.3 Hz, 4H), 1.56 (t, J=5.8 Hz, 2H), 0.83-0.42 (m, 4H). LCMS m/z 432.85 [M+H]⁺.

### Compound 427

### (3S,4R)-3-((2-(5,7-difluoro-2-(4-fluorophenyl)-1H-indol-3-yl)ethyl)amino)-4-hydroxypyrrolidin-2-one (427)

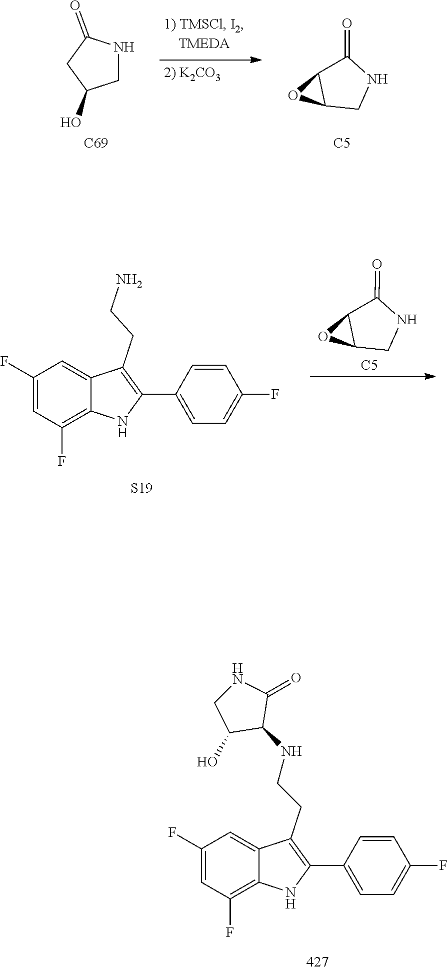

### Step 1. Synthesis of (1R,5R)-6-oxa-3-azabicyclo[3.1.0]hexan-2-one (C5)

A solution of (4S)-4-hydroxypyrrolidin-2-one (1.83 g, 18.1 mmol) C69 in DCM (50 mL) was cooled to −10° C. and N,N,N′,N′-tetramethylethane-1,2-diamine (11 mL, 72.89 mmol) was added. To this cooled reaction, chloro(trimethyl)silane (8 mL, 63.03 mmol) was then added dropwise as well as an additional portion of DCM (50 mL). The solution was then stirred at −10° C. for 30 minutes. at which point iodine (6 g, 23.64 mmol) was added and the slurry was stirred for 2 hours. The reaction was quenched with saturated aqueous Na₂SO₃ (80 mL) solution and stirred until the mixture forms 2 homogeneous layers. The layers were separated, and the aqueous layer was extracted with DCM. The combined organic layers were washed with 1 M HCl (80 mL), dried over Na₂SO₄, filtered, and concentrated to a yellow waxy solid. This solid was dissolved in EtOH (80 mL). To this solution was added K₂CO₃ (3.81 g, 27.57 mmol). The reaction was stirred at room temperature overnight. The reaction was filtered, and the precipitate was washed with DCM (40 mL) and the filtrate was concentrated. The resulting dark solid was rinsed with MeCN (40 mL) and filtered to obtain (1R,5R)-6-oxa-3-azabicyclo[3.1.0]hexan-2-one (2 g, 72% yield). LCMS m/z 128.78 [M+H]⁺.

### Step 2. Synthesis of (3S,4R)-3-((2-(5,7-difluoro-2-(4-fluorophenyl)-1H-indol-3-yl)ethyl)amino)-4-hydroxypyrrolidin-2-one (427)

(1R,5R)-6-oxa-3-azabicyclo[3.1.0]hexan-2-one S19 (50 mg, 0.5046 mmol) and 2-[5,7-difluoro-2-(4-fluorophenyl)-1H-indol-3-yl]ethanamine C5 (140 mg, 0.4823 mmol) were combined in EtOH (5 mL). The reaction was heated to 130° C. for and stirred for 20 minutes. The reaction was then concentrated and re-dissolved in DMSO (5 mL). Purification by reversed-phase HPLC (Method: C18 Waters Sunfire column (30×150 mm, 5 micron). Gradient: MeCN in H₂O with 0.1% trifluoroacetic acid) afforded the product (3S,4R)-3-((2-(5,7-difluoro-2-(4-fluorophenyl)-1H-indol-3-yl)ethyl)amino)-4-hydroxypyrrolidin-2-one (16.3 mg, 6.2%). ¹H NMR (300 MHz, Acetone-d₆) δ 10.92 (s, 1H), 7.99-7.76 (m, 2H), 7.50-7.37 (m, 1H), 7.37-7.19 (m, 2H), 6.86 (ddt, J=11.1, 9.7, 2.6 Hz, 1H), 4.93 (q, J=8.1 Hz, 1H), 4.14 (d, J=8.4 Hz, 1H), 3.98-3.79 (m, 1H), 3.74-3.41 (m, 4H), 3.28-3.01 (m, 1H). LCMS m/z 390.31 [M+H]⁺.

### Compound 428

### 2-[5,7-difluoro-2-(4-fluorophenyl)-1H-indol-3-yl]-N-methyl-ethanamine (428)

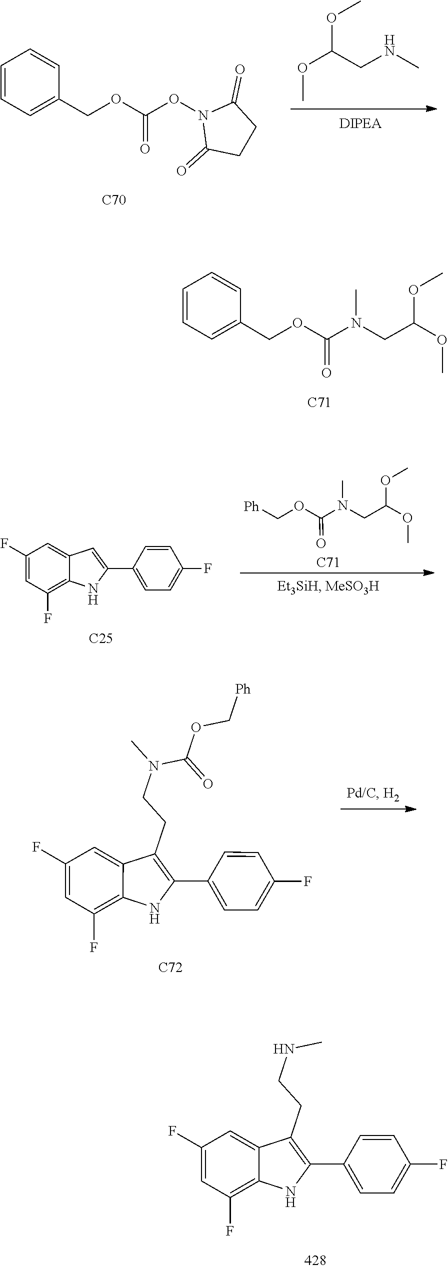

### Step 1. Synthesis of Benzyl N-(2,2-dimethoxyethyl)-N-methyl-carbamate (C71)

2,2-Dimethoxy-N-methyl-ethanamine (15 g, 125.9 mmol) was dissolved in DCM (100 mL) and to this solution was added DIPEA (22 mL, 126.3 mmol) and benzyl (2,5-dioxopyrrolidin-1-yl) carbonate C70 (32 g, 128.4 mmol). The reaction was stirred at room temperature for 3 hours and then washed with 1 M HCl (50 mL) followed by a wash with 1 M NaOH (50 mL). The resulting organic layer was washed with saturated NaCl (100 mL). The organics were concentrated to a yellow oil and used as it in the next step. Benzyl N-(2,2-dimethoxyethyl)-N-methyl-carbamate (28 g, 33%). LCMS m/z 254.11 [M+H]⁺.

### Step 2. Synthesis of benzyl N-[2-[5,7-difluoro-2-(4-fluorophenyl)-1H-indol-3-yl]ethyl]-N-methyl-carbamate (C72)

Benzyl N-(2,2-dimethoxyethyl)-N-methyl-carbamate C71 (5.3 g, 20.92 mmol) was dissolved in DCM (50 mL). To this solution was added triethylsilane (10 mL, 62.61 mmol). In a separate flask, 5,7-difluoro-2-(4-fluorophenyl)-1H-indole C25 (4.925 g, 19.92 mmol), methanesulfonic acid (3.5 mL, 53.94 mmol), and DCM (50 mL) were combined into a solution which was added dropwise to the solution containing the Benzyl N-(2,2-dimethoxyethyl)-N-methyl-carbamate C71. The reaction was stirred overnight at room temperature at which point it was concentrated and re-dissolved in DMSO (25 mL). Purified by 275 g reversed-phase chromatography (Column: C18. Gradient: 0-100% MeCN in water with 0.1% formic acid) to afford benzyl N-[2-[5,7-difluoro-2-(4-fluorophenyl)-1H-indol-3-yl]ethyl]-N-methyl-carbamate (4.5 g, 44%). LCMS m/z 437.25 [M+H]⁺.

### Step 3. Synthesis of 2-[5,7-difluoro-2-(4-fluorophenyl)-1H-indol-3-yl]-N-methyl-ethanamine (428)

Benzyl (2-(5,7-difluoro-2-(4-fluorophenyl)-1H-indol-3-yl)ethyl)(methyl)carbamate C₇₂ (900 mg, 2.053 mmol) was dissolved in EtOH (20 mL) and to this solution was added Pd/C (50 mg, 0.4698 mmol). A balloon containing H₂ was attached and the reaction was stirred overnight at room temperature. The reaction was then filtered, concentrated, and re-dissolved in DMSO (5 mL). The mixture was purified by reversed-phase chromatography (Column: C18. Gradient: 0-100% MeCN in water with 0.1% trifluoroacetic acid) to afford 2-[5,7-difluoro-2-(4-fluorophenyl)-1H-indol-3-yl]-N-methyl-ethanamine (Trifluoroacetate salt) (450 mg, 51%). ¹H NMR (300 MHz, Acetone-d₆) δ 10.84 (s, 1H), 7.84-7.73 (m, 2H), 7.48-7.34 (m, 1H), 7.35-7.22 (m, 2H), 6.84 (ddd, J=11.5, 9.7, 2.1 Hz, 1H), 3.49-3.32 (m, 4H), 2.82 (s, 3H). LCMS m/z 305.47 [M+H]⁺.

### Compound 429

### 2-[5,7-difluoro-2-(4-fluorophenyl)-1H-indol-3-yl]-N,N-dimethyl-ethanamine (429)

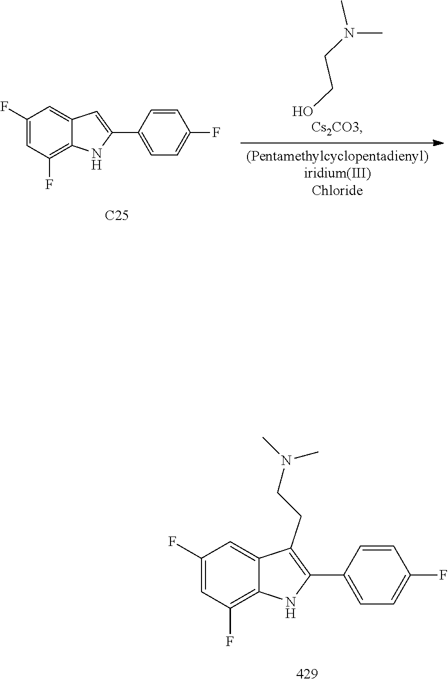

### Preparation of 2-[5,7-difluoro-2-(4-fluorophenyl)-1H-indol-3-yl]-N,N-dimethyl-ethanamine (429)

To a microwave vial was added 5,7-difluoro-2-(4-fluorophenyl)-1H-indole C25 (100 mg, 0.3882 mmol), 2-(dimethylamino)ethanol (117 μL, 1.164 mmol), Cs₂CO₃ (139 mg, 0.4266 mmol, and (pentamethylcyclopentadienyl)iridium(III) Chloride (4 mg, 0.01004 mmol). Heated the reaction mix at 150° C. for 48 hours. Cooled the reaction mix to room temperature. Purified by reversed-phase chromatography (Column: C18. Gradient: 5-95% MeCN in water with 0.1% TFA) to afford 2-[5,7-difluoro-2-(4-fluorophenyl)-1H-indol-3-yl]-N,N-dimethyl-ethanamine (Trifluoroacetate salt) (68.6 mg, 39%). ¹H NMR (300 MHz, DMSO-d₆) δ 11.90 (s, 1H), 9.51 (s, 1H), 7.74-7.64 (m, 2H), 7.45-7.33 (m, 3H), 7.11-6.97 (m, 1H), 3.34-3.22 (m, 2H), 3.16-3.04 (m, 2H), 2.84 (s, 6H). LCMS m/z 319.19 [M+H]⁺.

### Compound 430

### tert-butyl N-[2-[2-(4-fluorophenyl)-5-methoxy-1H-indol-3-yl]ethyl]carbamate (430)

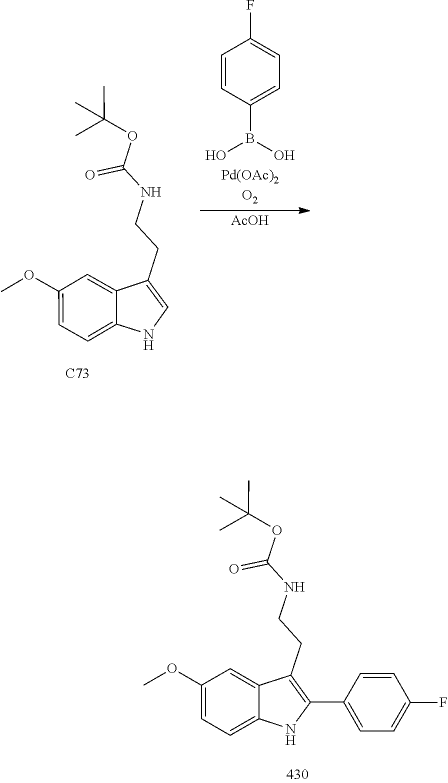 [SMILES: "AcOH" ; COc1ccc2[nH]cc(CCNC(=O)OC(C)(C)C)c2c1 ; OB(O)c1ccc(F)cc1 ; "C73" ; COc1ccc2[nH]c(-c3ccc(F)cc3)c(CCNC(=O)OC(C)(C)C)c2c1]

### Preparation of tert-butyl N-[2-[2-(4-fluorophenyl)-5-methoxy-1H-indol-3-yl]ethyl]carbamate (430)

A solution of tert-butyl N-[2-(5-methoxy-1H-indol-3-yl)ethyl]carbamate C73 (30 mg, 0.1033 mmol) in AcOH (2 mL) was treated with 4-fluorophenylboronic acid (22 mg, 0.1572 mmol) and palladium acetate (5 mg, 0.02227 mmol), and 02 balloon. The reaction was stirred at room temperature overnight. The reaction was concentrated and purified by silica gel chromatography (Gradient: 0-100% EtOAc in heptane) to afford tert-butyl N-[2-[2-(4-fluorophenyl)-5-methoxy-1H-indol-3-yl]ethyl]carbamate 430 (14.9 mg, 35%). ¹H NMR (300 MHz, Methanol-d₄) δ 7.66-7.57 (m, 2H), 7.27-7.08 (m, 4H), 6.77 (dd, J=8.7, 2.4 Hz, 1H), 3.85 (s, 3H), 3.37-3.32 (m, 2H), 2.99 (dd, J=8.7, 6.4 Hz, 2H), 1.41 (s, 9H). LCMS m/z 384.24 [M+H]⁺

### Compound 431

### N-[2-[2-(4-fluorophenyl)-1H-indol-3-yl]ethyl]acetamide (431)

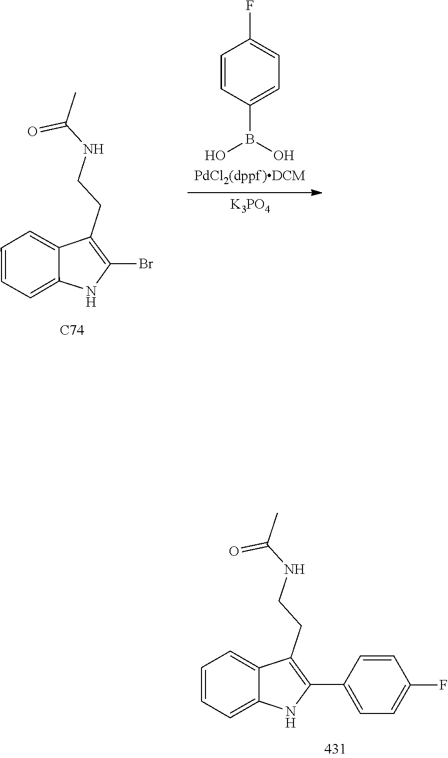

### Preparation of N-[2-[2-(4-fluorophenyl)-1H-indol-3-yl]ethyl]acetamide (431)

A solution of N-[2-(2-bromo-1H-indol-3-yl)ethyl]acetamide C74 (65 mg, 0.2312 mmol), (4-fluorophenyl)boronic acid (50 mg, 0.3573 mmol), and K₃PO₄ (350 μL of 2 M, 0.7000 mmol) in dioxane (800 μL) was flushed with nitrogen, added ferrous; cyclopenta-1,4-dien-1-yl(diphenyl)phosphane; dichloromethane; dichloropalladium (40 mg, 0.04898 mmol) and stirred at 100° C. for 2 hours. The reaction mixture was then added to water, extracted with EtOAc (3 times), dried over sodium sulfate, filtered, and solvent was removed under reduced pressure. Purification by reversed-phase HPLC (Method: C18 Waters Sunfire column (30×150 mm, 5 micron). Gradient: MeCN in H₂O with 0.1% trifluoroacetic acid) afforded N-[2-[2-(4-fluorophenyl)-1H-indol-3-yl]ethyl]acetamide (30 mg, 42%). LCMS m/z 296.8 [M+H]⁺.

### Compound 432

### 2-[5,7-difluoro-2-(4-fluorophenyl)-1H-indol-3-yl]ethanamine (432)

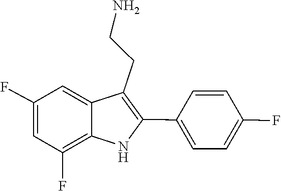 [SMILES: NCCc1c(-c2ccc(F)cc2)[nH]c2c(F)cc(F)cc12]

Compound 432 (=S19) was prepared as described for S19.

### Compound 433

### 2-[5,7-difluoro-2-(4-fluorophenyl)-1H-indol-3-yl]ethanol (433)

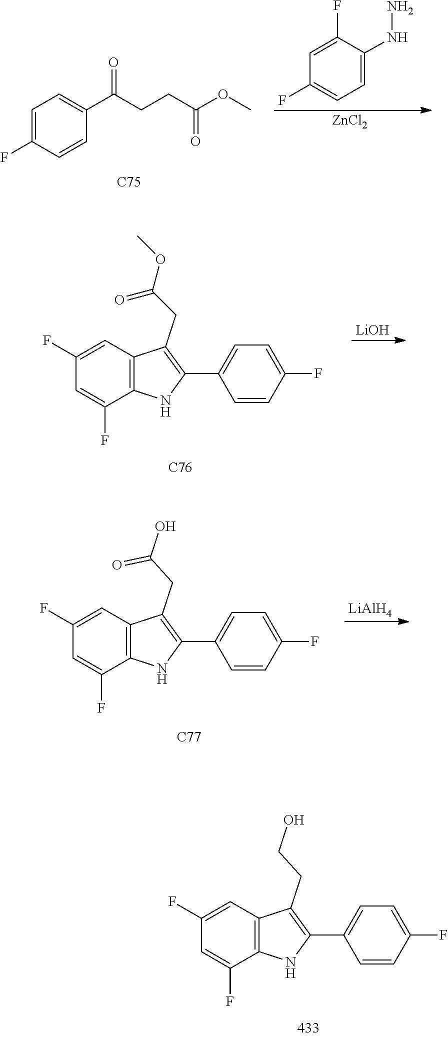

### Step 1. Synthesis of methyl 2-[5,7-difluoro-2-(4-fluorophenyl)-1H-indol-3-yl]acetate (C76)

To a mixture of methyl 4-(4-fluorophenyl)-4-oxo-butanoate C75 (1 g, 5 mmol) and (2,4-difluorophenyl)hydrazine hydrochloride (1.72 g, 9.53 mmol) in acetic acid (13 mL) and toluene (13 mL) was added zinc chloride (3.1 g, 23 mmol). The mixture was heated for 24 hours (at ˜115° C.) and concentrated in vacuo. The residue was partitioned between EtOAc and water. The aqueous layer was extracted with EtOAc, and the combined organic layer was washed with brine, dried over sodium sulfate, filtered, and concentrated in vacuo. The residue was purified by silica gel chromatography (Gradient: 0-20% EtOAc in heptane) to give the product (356.4 mg, 22%). ¹H NMR (300 MHz, Chloroform-d) δ 8.23 (s, 1H), 7.68-7.56 (m, 2H), 7.24-7.15 (m, 2H), 7.13-7.05 (m, 1H), 6.75 (ddd, J=10.8, 9.5, 2.2 Hz, 1H), 3.74 (d, J=3.3 Hz, 5H). LCMS m/z 320.14 [M+H]⁺.

### Step 2. Synthesis of 2-fluoro-6-[2-(4-fluorophenyl)ethynyl]-4-(trifluoromethyl)aniline 2-[5,7-difluoro-2-(4-fluorophenyl)-1H-indol-3-yl]acetic acid (C77)

To a solution of methyl 2-[5,7-difluoro-2-(4-fluorophenyl)-1H-indol-3-yl]acetate C76 (351.6 mg, 1.046 mmol) in THF (2 mL), MeOH (2 mL), and water (1 mL) was added LiOH (55 mg, 2.3 mmol). The mixture was stirred at room temperature for 2.5 hours. The mixture was then concentrated in vacuo, and the residue was acidified to pH 3 with 1 M HCl (aqueous). The aqueous layer was extracted with EtOAc, and the organic layer was washed with brine, dried over sodium sulfate, filtered, and concentrated in vacuo to afford the product (317.5 mg, 94%), which was used in subsequent reactions without further purification. ¹H NMR (300 MHz, DMSO-d₆) δ 12.45 (s, 1H), 11.86 (s, 1H), 7.80-7.65 (m, 2H), 7.45-7.33 (m, 2H), 7.19 (dt, J=9.5, 2.6 Hz, 1H), 7.00 (ddd, J=11.2, 9.8, 2.2 Hz, 1H), 3.60 (s, 2H). LCMS m/z 306.14 [M+H]⁺.

### Step 3. Synthesis of 2-[5,7-difluoro-2-(4-fluorophenyl)-1H-indol-3-yl]ethanol (433)

To a solution of 2-[5,7-difluoro-2-(4-fluorophenyl)-1H-indol-3-yl]acetic acid C77 (62.1 mg, 0.193 mmol) in THF (1.4 mL) at 0° C. under a nitrogen atmosphere was added lithium aluminum hydride (387 μL of 2 M in THF, 0.774 mmol). The ice bath was removed, and the mixture was allowed to warm to room temperature. The mixture was then refluxed for 2.5 hours. The mixture was cooled to room temperature, and the reaction was quenched at 0° C. by the addition of saturated aqueous solution of sodium potassium L(+)-tartrate tetrahydrate followed by ethyl acetate. The aqueous layer was extracted with ethyl acetate, and the combined organic layer was washed with brine, dried over sodium sulfate, filtered, and concentrated in vacuo to afford the product (30.6 mg, 50%). ¹H NMR (300 MHz, Methanol-d₄) δ 7.73-7.64 (m, 2H), 7.27-7.18 (m, 2H), 7.10 (dd, J=9.4, 2.2 Hz, 1H), 6.72 (ddd, J=11.1, 9.6, 2.2 Hz, 1H), 3.79 (t, J=7.3 Hz, 2H), 3.01 (t, J=7.3 Hz, 2H). LCMS m/z 292.14 [M+H]⁺.

### Compound 434

### 2-[5,7-difluoro-2-(4-fluorophenyl)-1H-indol-3-yl]ethyl N-[2-hydroxy-1-(hydroxymethyl)-1-methyl-ethyl]carbamate (434)

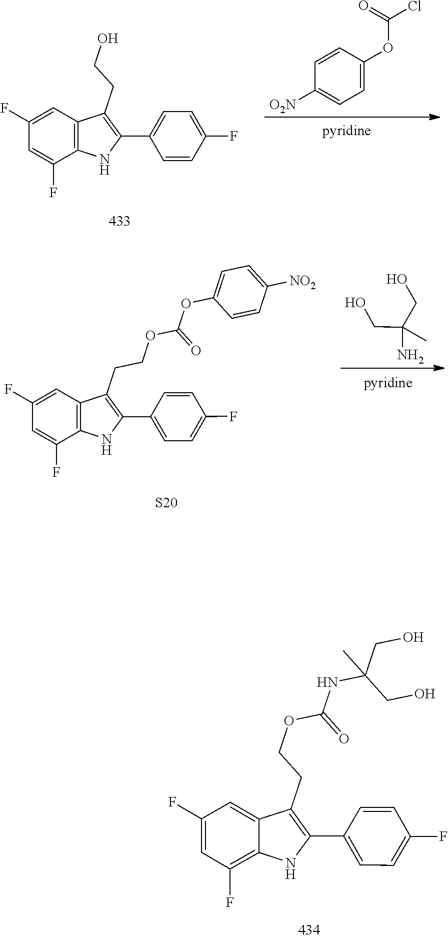

### Step 1. Synthesis of 2-[5,7-difluoro-2-(4-fluorophenyl)-1H-indol-3-yl]ethyl (4-nitrophenyl) carbonate (S20)

To a solution of 2-[5,7-difluoro-2-(4-fluorophenyl)-1H-indol-3-yl]ethanol 433 (3000 mg, 6.90 mmol) in DCM (50 mL) was added (4-nitrophenyl) carbonochloridate (2 g, 9 mmol) followed by pyridine (1 g, 13 mmol). The reaction mixture was stirred for 3 hours. The mixture was concentrated in vacuo and partitioned between EtOAc and water. The organic layer separated and washed with 2 M NaOH) and brine. The organic layer was dried over sodium sulfate, filtered, and concentrated in vacuo to give the product (2.7 g, 25%), which was used in the next step without additional purification. LCMS m/z 457.47 [M+H]⁺.

### Step 2. Synthesis of 2-[5,7-difluoro-2-(4-fluorophenyl)-1H-indol-3-yl]ethyl N-[2-hydroxy-1-(hydroxymethyl)-1-methyl-ethyl]carbamate (434)

To a solution of 2-[5,7-difluoro-2-(4-fluorophenyl)-1H-indol-3-yl]ethyl (4-nitrophenyl) carbonate S20 (50 mg, 0.1 mmol) in DMF (2 mL) was added 2-amino-2-methyl-propane-1,3-diol (13.8 mg, 0.13 mmol), followed by pyridine (17 mg, 0.22 mmol). The mixture was heated to 80° C. for 3 h. The mixture was then filtered and purified by reversed-phase HPLC (Method: C18 Waters Sunfire column (30×150 mm, 5 micron). Gradient: MeCN in H₂O with 0.1% trifluoroacetic acid) to afford the product (16.5 mg, 28%). LCMS m/z 423.14 [M+H]⁺.

### Compounds 435-502

Compounds 435-502 (see Table 13) were prepared from intermediate S20 using the appropriate amine and using the carbamate coupling method as described for compound 434. Amines were obtained from commercial sources. Any modifications to methods are noted in Table 13 and accompanying footnotes.

TABLE 13
Structure and physicochemical data for compounds 435-502

| | | | ¹H NMR; LCMS m/z |
|---|---|---|---|
| Compound | Product | Amine | [M + H]⁺ |
| 435 | 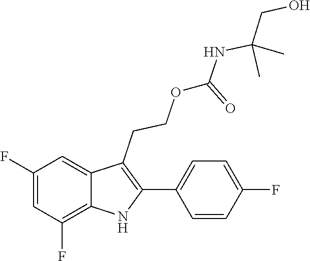 [SMILES: CC(C)(CO)NC(=O)OCCc1c(-c2ccc(F)cc2)[nH]c2c(F)cc(F)cc12] | 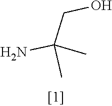 | ¹H NMR (300 MHz, Acetone-d₆) δ 10.76 (s, 1H), 8.03-7.52 (m, 2H), 7.30 (td, J = 9.2, 2.5 Hz, 3H), 6.84 (ddd, J = 11.1, 9.7, 2.2 Hz, 1H), 5.74 (s, 1H), 4.25 (t, J = 7.3 Hz, 2H), 3.51 (s, 2H), 3.12 (t, J = 7.3 Hz, 2H), 1.25 (s, 6H); LCMS m/z 407.14 [M + H]⁺ |
| 436 | 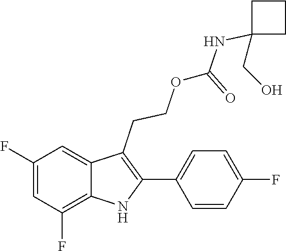 [SMILES: O=C(NC1(CO)CCC1)OCCc1c(-c2ccc(F)cc2)[nH]c2c(F)cc(F)cc12] | 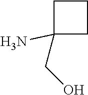 | LCMS m/z 419.16 [M + H]⁺ |
| 437 | 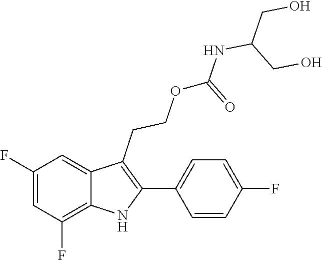 [SMILES: O=C(NC(CO)CO)OCCc1c(-c2ccc(F)cc2)[nH]c2c(F)cc(F)cc12] | 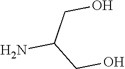 | LCMS m/z 409.1 [M + H]⁺ |
| 438 | 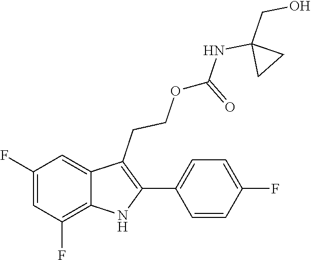 [SMILES: O=C(NC1(CO)CC1)OCCc1c(-c2ccc(F)cc2)[nH]c2c(F)cc(F)cc12] | 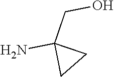 | LCMS m/z 405.22 [M + H]⁺ |
| 439 | 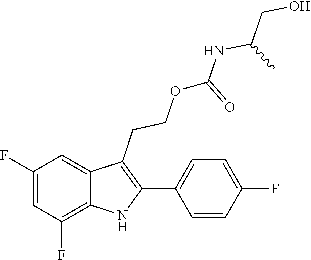 [SMILES: CC(CO)NC(=O)OCCc1c(-c2ccc(F)cc2)[nH]c2c(F)cc(F)cc12] | 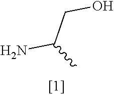 | LCMS m/z 393.3 [M + H]⁺ |
| 440 | 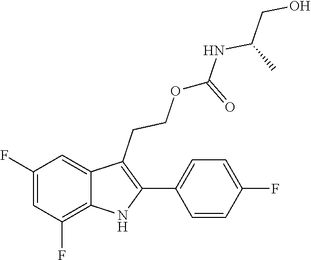 [SMILES: C[C@@H](CO)NC(=O)OCCc1c(-c2ccc(F)cc2)[nH]c2c(F)cc(F)cc12] | 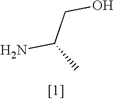 | ¹H NMR (300 MHz, Acetone-d₆) δ 10.76 (s, 1H), 7.99-7.54 (m, 2H), 7.34-7.16 (m, 3H), 6.84 (ddd, J = 11.1, 9.7, 2.2 Hz, 1H), 5.92 (s, 1H), 4.29 (td, J = 7.5, 1.9 Hz, 2H), 3.83-3.59 (m, 1H), 3.49 (qd, J = 10.6, 5.5 Hz, 2H), 3.13 (t, J = 7.3 Hz, 3H), 1.13 (d, J = 6.7 Hz, 3H); LCMS m/z 393.1 [M + H]⁺ |
| 441 | 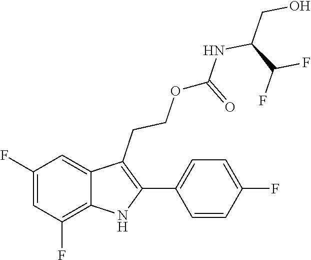 [SMILES: O=C(N[C@@H](CO)C(F)F)OCCc1c(-c2ccc(F)cc2)[nH]c2c(F)cc(F)cc12] | 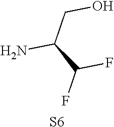 | LCMS m/z 429.03 [M + H]⁺ |
| 442 | 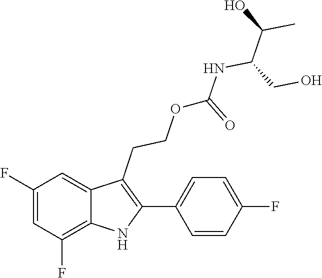 [SMILES: C[C@H](O)[C@H](CO)NC(=O)OCCc1c(-c2ccc(F)cc2)[nH]c2c(F)cc(F)cc12] | 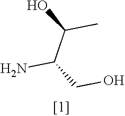 | ¹H NMR (300 MHz, Acetone-d₆) δ 10.76 (s, 1H), 8.10-7.62 (m, 2H), 7.51-7.13 (m, 3H), 6.83 (ddd, J = 11.1, 9.7, 2.2 Hz, 1H), 5.69 (d, J = 8.8 Hz, 1H), 4.48-4.19 (m, 2H), 4.08 (qd, J = 6.5, 2.6 Hz, 1H), 3.66 (h, J = 6.0, 5.5 Hz, 3H), 3.61-3.37 (m, 2H), 3.15 (t, J = 7.3 Hz, 2H), 1.13 (d, J = 6.4 Hz, 3H); LCMS m/z 423.23 [M + H]⁺ |
| 443 | 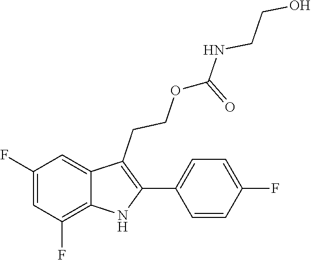 [SMILES: O=C(NCCO)OCCc1c(-c2ccc(F)cc2)[nH]c2c(F)cc(F)cc12] | | LCMS m/z 379.16 [M + H]⁺ |
| 444 | 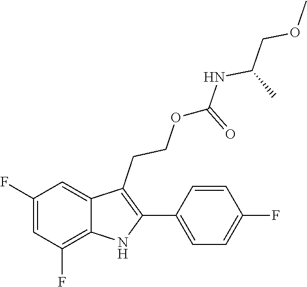 [SMILES: COC[C@H](C)NC(=O)OCCc1c(-c2ccc(F)cc2)[nH]c2c(F)cc(F)cc12] | 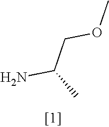 | ¹H NMR (300 MHz, Acetone-d₆) δ 10.76 (s, 1H), 7.90-7.61 (m, 2H), 7.42-7.15 (m, 3H), 6.84 (ddd, J = 11.2, 9.7, 2.2 Hz, 1H), 4.29 (t, J = 7.3 Hz, 2H), 3.79 (d, J = 6.5 Hz, 1H), 3.43-3.19 (m, 5H), 3.13 (t, J = 7.3 Hz, 2H), 1.11 (d, J = 6.8 Hz, 3H); LCMS m/z 407.01 [M + H]⁺ |
| 445 | 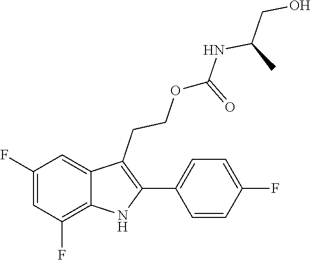 [SMILES: C[C@H](CO)NC(=O)OCCc1c(-c2ccc(F)cc2)[nH]c2c(F)cc(F)cc12] | 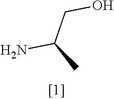 | LCMS m/z 393.33 [M + H]⁺ |
| 446 | 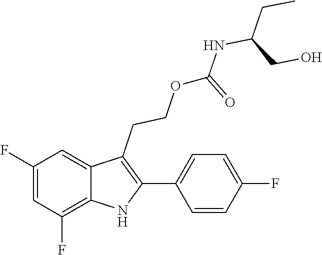 [SMILES: CC[C@@H](CO)NC(=O)OCCc1c(-c2ccc(F)cc2)[nH]c2c(F)cc(F)cc12] | 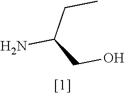 | ¹H NMR (300 MHz, Acetone-d₆) δ 10.76 (s, 1H), 7.92-7.63 (m, 2H), 7.30 (ddd, J = 8.9, 5.4, 2.9 Hz, 3H), 6.84 (ddd, J = 11.3, 9.7, 2.2 Hz, 1H), 5.87 (s, 1H), 4.30 (tq, J = 6.9, 3.5 Hz, 2H), 3.50 (d, J = 11.3 Hz, 4H), 3.13 (t, J = 7.3 Hz, 3H), 1.64 (s, 1H), 1.44 (dt, J = 13.8, 7.3 Hz, 1H), 0.91 (t, J = 7.5 Hz, 3H); LCMS m/z 407.14 [M + H]⁺ |
| 447 | 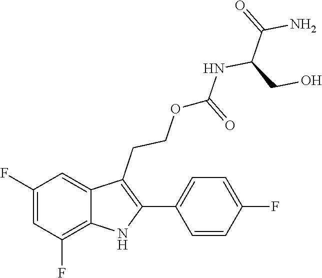 [SMILES: NC(=O)[C@@H](CO)NC(=O)OCCc1c(-c2ccc(F)cc2)[nH]c2c(F)cc(F)cc12] | 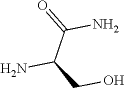 | LCMS m/z 422.09 [M + H]⁺ |
| 448 | 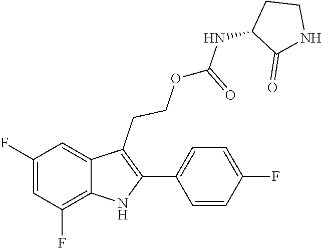 [SMILES: O=C(N[C@@H]1CCNC1=O)OCCc1c(-c2ccc(F)cc2)[nH]c2c(F)cc(F)cc12] | 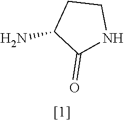 | LCMS m/z 418.02 [M + H]⁺ |
| 449 | 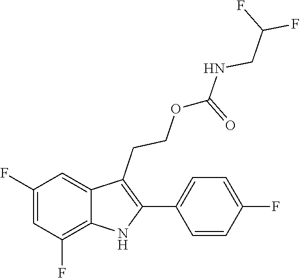 [SMILES: O=C(NCC(F)F)OCCc1c(-c2ccc(F)cc2)[nH]c2c(F)cc(F)cc12] | 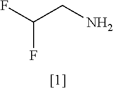 | ¹H NMR (300 MHz, Acetone-d₆) δ 10.78 (s, 1H), 7.80 (ddd, J = 8.6, 5.3, 2.4 Hz, 2H), 7.31 (ddt, J = 8.8, 5.2, 2.7 Hz, 3H), 6.84 (ddd, J = 11.5, 9.6, 2.2 Hz, 1H), 6.64 (s, 1H), 6.19-5.62 (m, 1H), 4.34 (t, J = 7.3 Hz, 2H), 3.50 (tt, J = 15.0, 5.4 Hz, 2H), 3.16 (t, J = 7.3 Hz, 2H); LCMS m/z 399.1 [M + H]⁺ |
| 450 | 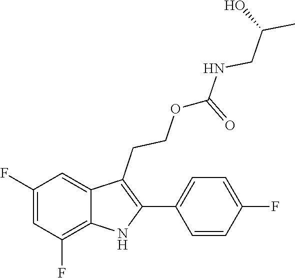 [SMILES: C[C@@H](O)CNC(=O)OCCc1c(-c2ccc(F)cc2)[nH]c2c(F)cc(F)cc12] | 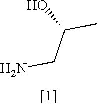 | ¹H NMR (300 MHz, Acetone-d₆) δ 10.76 (s, 1H), 7.95-7.66 (m, 2H), 7.42-7.17 (m, 3H), 6.84 (ddd, J = 11.1, 9.7, 2.2 Hz, 1H), 5.91 (s, 1H), 4.29 (td, J = 7.5, 1.9 Hz, 2H), 3.70 (s, 1H), 3.48 (qd, J = 10.6, 5.5 Hz, 2H), 3.13 (t, J = 7.3 Hz, 2H), 1.13 (d, J = 6.7 Hz, 3H); LCMS m/z 393.17 [M + H]⁺ |
| 451 | 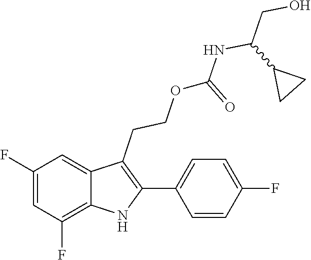 [SMILES: O=C(NC(CO)C1CC1)OCCc1c(-c2ccc(F)cc2)[nH]c2c(F)cc(F)cc12] | 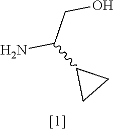 | LCMS m/z 419.16 [M + H]⁺ |
| 452 | 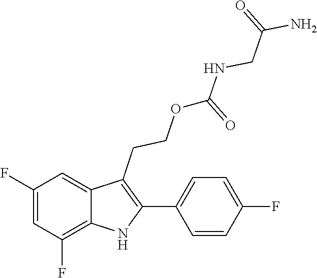 [SMILES: NC(=O)CNC(=O)OCCc1c(-c2ccc(F)cc2)[nH]c2c(F)cc(F)cc12] | 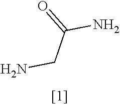 | LCMS m/z 392.22 [M + H]⁺ |
| 453 | 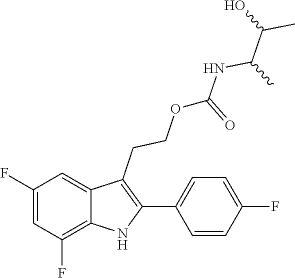 [SMILES: CC(O)C(C)NC(=O)OCCc1c(-c2ccc(F)cc2)[nH]c2c(F)cc(F)cc12] | 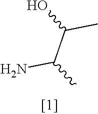 | ¹H NMR (300 MHz, Acetone-d₆) δ 10.76 (s, 1H), 7.99-7.64 (m, 2H), 7.41-7.15 (m, 3H), 6.84 (ddd, J = 11.0, 9.6, 2.2 Hz, 1H), 5.81 (d, J = 51.0 Hz, 1H), 4.30 (t, J = 7.3 Hz, 2H), 3.91-3.34 (m, 3H), 3.14 (d, J = 14.6 Hz, 3H), 1.26- 0.72 (m, 6H); LCMS m/z 407.11 [M + H]⁺ |
| 454 | 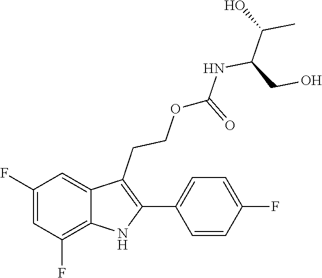 [SMILES: C[C@@H](O)[C@@H](CO)NC(=O)OCCc1c(-c2ccc(F)cc2)[nH]c2c(F)cc(F)cc12] | 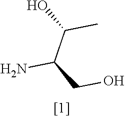 | LCMS m/z 422.97 [M + H]⁺ |
| 455 | 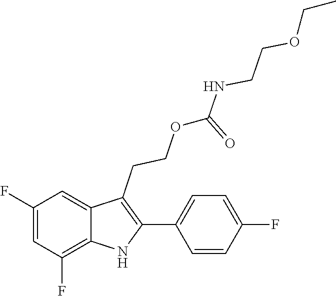 [SMILES: CCOCCNC(=O)OCCc1c(-c2ccc(F)cc2)[nH]c2c(F)cc(F)cc12] | 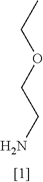 | LCMS m/z 407.37 [M + H]⁺ |
| 456 | 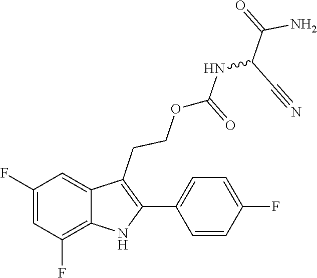 [SMILES: N#CC(NC(=O)OCCc1c(-c2ccc(F)cc2)[nH]c2c(F)cc(F)cc12)C(N)=O] | 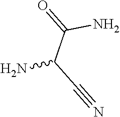 | LCMS m/z 417.31 [M + H]⁺ |
| 457 | 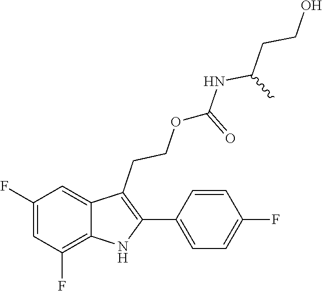 [SMILES: CC(CCO)NC(=O)OCCc1c(-c2ccc(F)cc2)[nH]c2c(F)cc(F)cc12] | 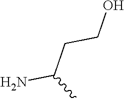 | LCMS m/z 407.34 [M + H]⁺ |
| 458 | 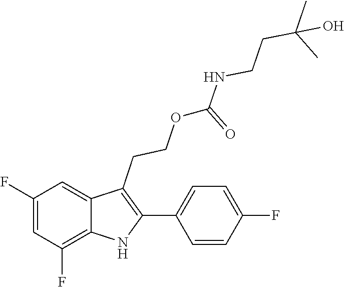 [SMILES: CC(C)(O)CCNC(=O)OCCc1c(-c2ccc(F)cc2)[nH]c2c(F)cc(F)cc12] | 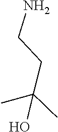 | LCMS m/z 421.18 [M + H]⁺ |
| 459 | 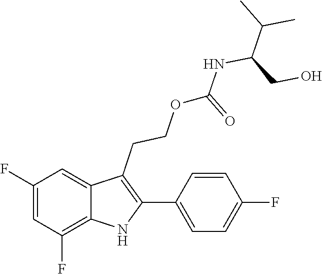 [SMILES: CC(C)[C@@H](CO)NC(=O)OCCc1c(-c2ccc(F)cc2)[nH]c2c(F)cc(F)cc12] | 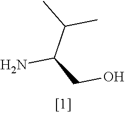 | ¹H NMR (300 MHz, Acetone-d₆) δ 10.76 (s, 1H), 7.96-7.58 (m, 2H), 7.45-7.16 (m, 3H), 6.84 (ddd, J = 11.1, 9.7, 2.2 Hz, 1H), 5.84 (d, J = 9.1 Hz, 1H), 4.55-4.13 (m, 2H), 3.68-3.34 (m, 4H), 3.13 (t, J = 7.3 Hz, 2H), 1.91 (h, J = 6.7 Hz, 1H), 0.91 (dd, J = 8.9, 6.8 Hz, 6H); LCMS m/z 421.35 [M + H]⁺ |
| 460 | 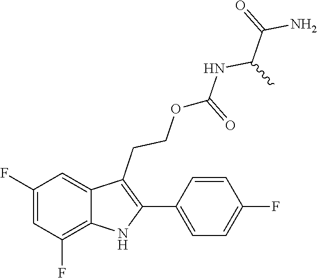 [SMILES: CC(NC(=O)OCCc1c(-c2ccc(F)cc2)[nH]c2c(F)cc(F)cc12)C(N)=O] | 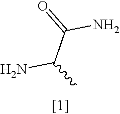 | LCMS m/z 406.03 [M + H]⁺ |
| 461 | 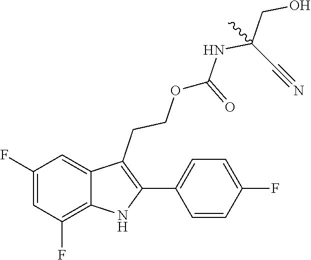 [SMILES: CC(C#N)(CO)NC(=O)OCCc1c(-c2ccc(F)cc2)[nH]c2c(F)cc(F)cc12] | 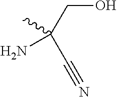 | LCMS m/z 418.12 [M + H]⁺ |
| 462 | 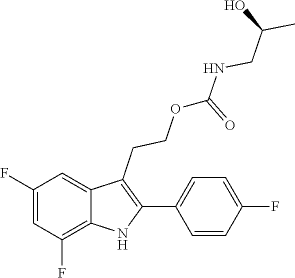 [SMILES: C[C@H](O)CNC(=O)OCCc1c(-c2ccc(F)cc2)[nH]c2c(F)cc(F)cc12] | 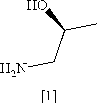 | LCMS m/z 393.13 [M + H]⁺ |
| 463 | 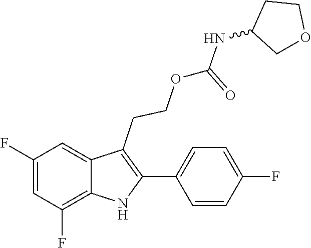 [SMILES: O=C(NC1CCOC1)OCCc1c(-c2ccc(F)cc2)[nH]c2c(F)cc(F)cc12] | 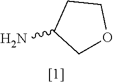 | ¹H NMR (300 MHz, Acetone-d₆) δ 10.76 (s, 1H), 7.79 (ddd, J = 8.6, 5.5, 2.5 Hz, 2H), 7.30 (td, J = 9.0, 2.6 Hz, 3H), 6.84 (ddd, J = 11.5, 9.7, 2.3 Hz, 1H), 6.51-6.27 (m, 1H), 4.47-3.97 (m, 3H), 3.96-3.59 (m, 3H), 3.53 (dd, J = 9.3, 3.9 Hz, 1H), 3.13 (t, J = 7.3 Hz, 2H), 2.13 (dt, J = 10.7, 5.2 Hz, 1H), 1.92-1.66 (m, |
| | | | 1H); LCMS m/z |
| | | | 404.99 [M + H]⁺ |
| 464 | 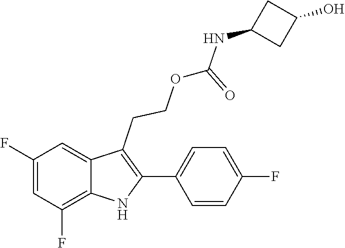 [SMILES: O=C(N[C@H]1C[C@H](O)C1)OCCc1c(-c2ccc(F)cc2)[nH]c2c(F)cc(F)cc12] | 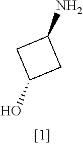 | LCMS m/z 404.93 [M + H]⁺ |
| 465 | 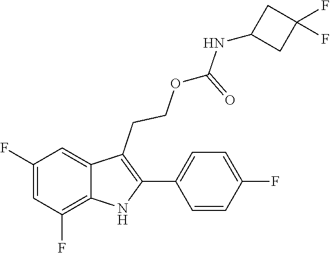 [SMILES: O=C(NC1CC(F)(F)C1)OCCc1c(-c2ccc(F)cc2)[nH]c2c(F)cc(F)cc12] | 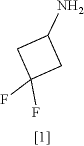 | ¹H NMR (300 MHz, Acetone-d₆) δ 10.76 (s, 1H), 7.92-7.68 (m, 2H), 7.29 (td, J = 8.8, 2.1 Hz, 3H), 6.84 (ddd, J = 11.1, 9.6, 2.2 Hz, 1H), 4.31 (t, J = 7.2 Hz, 2H), 4.02 (s, 1H), 3.15 (t, J = 7.2 Hz, 2H), 2.88 (h, J = 12.0, 11.3 Hz, 2H), 2.62 (d, J = 18.0 Hz, 2H); LCMS m/z 425.12 [M + H]⁺ |
| 466 | 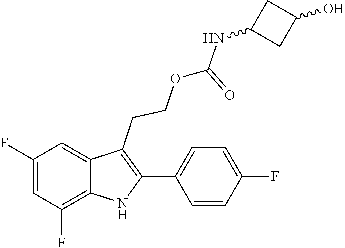 [SMILES: O=C(NC1CC(O)C1)OCCc1c(-c2ccc(F)cc2)[nH]c2c(F)cc(F)cc12] | 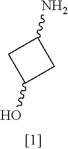 | ¹H NMR (300 MHz, Acetone-d₆) δ 10.76 (s, 1H), 7.93-7.62 (m, 2H), 7.38-7.11 (m, 3H), 6.84 (dddd, J = 10.9, 9.7, 2.2, 0.9 Hz, 1H), 4.28 (q, J = 7.3 Hz, 3H), 4.00-3.78 (m, 1H), 3.63 (d, J = 9.8 Hz, 1H), 3.11 (d, J = 6.7 Hz, 2H), 2.99- 2.74 (m, 1H), 2.68- 2.40 (m, 2H), 2.25 (s, 1H), 1.84 (d, J = 10.8 Hz, 1H); LCMS m/z |
| | | | 405.19 [M + H]⁺ |
| 467 | 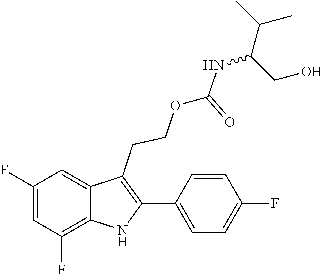 [SMILES: CC(C)C(CO)NC(=O)OCCc1c(-c2ccc(F)cc2)[nH]c2c(F)cc(F)cc12] | 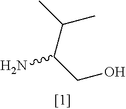 | LCMS m/z 421.31 [M + H]⁺ |
| 468 | 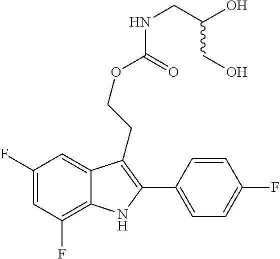 [SMILES: O=C(NCC(O)CO)OCCc1c(-c2ccc(F)cc2)[nH]c2c(F)cc(F)cc12] | 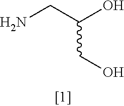 | LCMS m/z 409.03 [M + H]⁺ |
| 469 | 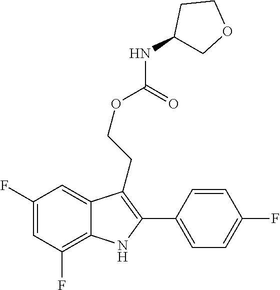 [SMILES: O=C(N[C@H]1CCOC1)OCCc1c(-c2ccc(F)cc2)[nH]c2c(F)cc(F)cc12] | 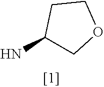 | LCMS m/z 404.99 [M + H]⁺ |
| 470 | 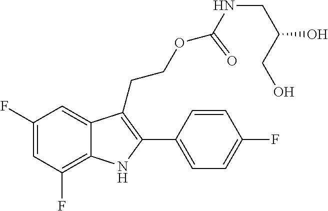 [SMILES: O=C(NC[C@H](O)CO)OCCc1c(-c2ccc(F)cc2)[nH]c2c(F)cc(F)cc12] | 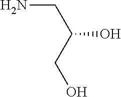 | LCMS m/z 409.23 [M + H]⁺ |
| 471 | 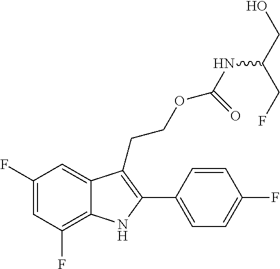 [SMILES: O=C(NC(CO)CF)OCCc1c(-c2ccc(F)cc2)[nH]c2c(F)cc(F)cc12] | 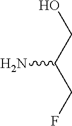 | LCMS m/z 411.31 [M + H]⁺ |
| 472 | 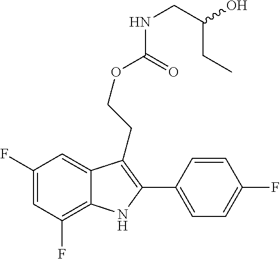 [SMILES: CCC(O)CNC(=O)OCCc1c(-c2ccc(F)cc2)[nH]c2c(F)cc(F)cc12] | 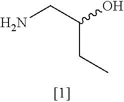 | ¹H NMR (300 MHz, Acetone-d₆) δ 10.76 (s, 1H), 7.80 (ddd, J = 8.3, 5.3, 2.4 Hz, 2H), 7.30 (ddt, J = 8.9, 6.4, 2.3 Hz, 3H), 6.84 (ddd, J = 10.2, 9.0, 2.5 Hz, 1H), 6.07 (s, 1H), 4.30 (t, J = 7.3 Hz, 2H), 3.64 (d, J = 64.4 Hz, 2H), 3.30-2.51 (m, 4H), 1.40 (ddt, J = 20.9, 13.7, 7.1 Hz, 2H), 0.93 (t, J = 6.9 Hz, 3H); LCMS m/z 407.04 [M + H]⁺ |
| 473 | 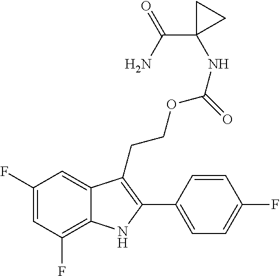 [SMILES: NC(=O)C1(NC(=O)OCCc2c(-c3ccc(F)cc3)[nH]c3c(F)cc(F)cc23)CC1] | 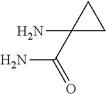 | LCMS m/z 418.32 [M + H]⁺ |
| 474 | 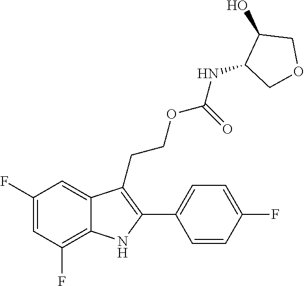 [SMILES: O=C(N[C@H]1COC[C@@H]1O)OCCc1c(-c2ccc(F)cc2)[nH]c2c(F)cc(F)cc12] | 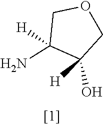 | LCMS m/z 421.05 [M + H]⁺ |
| 475 | 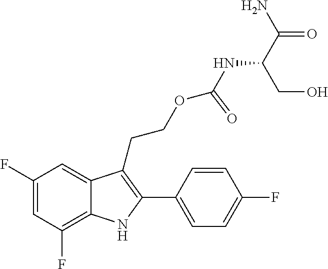 [SMILES: NC(=O)[C@H](CO)NC(=O)OCCc1c(-c2ccc(F)cc2)[nH]c2c(F)cc(F)cc12] | 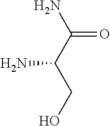 | LCMS m/z 422.29 [M + H]⁺ |
| 476 | 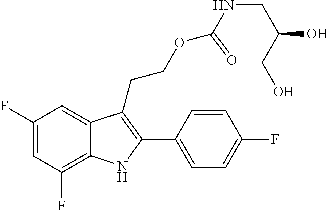 [SMILES: O=C(NC[C@@H](O)CO)OCCc1c(-c2ccc(F)cc2)[nH]c2c(F)cc(F)cc12] | 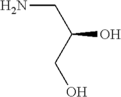 | LCMS m/z 409.26 [M + H]⁺ |
| 477 | 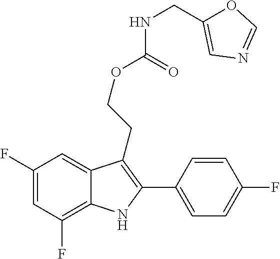 [SMILES: O=C(NCc1cnco1)OCCc1c(-c2ccc(F)cc2)[nH]c2c(F)cc(F)cc12] | 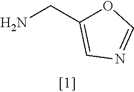 | ¹H NMR (300 MHz, Acetone-d₆) δ 10.76 (s, 1H), 8.01 (s, 1H), 7.87-7.70 (m, 2H), 7.40-7.21 (m, 3H), 7.00-6.68 (m, 3H), 4.57-4.19 (m, 4H), 3.15 (t, J = 7.3 Hz, 2H); LCMS m/z 416.13 [M + H]⁺ |
| 478 | 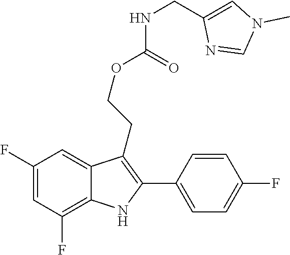 [SMILES: Cn1cnc(CNC(=O)OCCc2c(-c3ccc(F)cc3)[nH]c3c(F)cc(F)cc23)c1] | 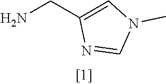 | ¹H NMR (300 MHz, Acetic Acid-d₄) δ 10.75 (s, 1H), 8.72 (s, 1H), 7.90-7.71 (m, 2H), 7.65 (s, 1H), 7.42 (s, 1H), 7.38-7.20 (m, 3H), 6.83 (ddd, J = 11.7, 9.8, 2.2 Hz, 1H), 4.51-4.22 (m, 4H), 4.02 (s, 3H), 3.11 (t, J = 7.2 Hz, 2H); LCMS m/z 428.97 [M + H]⁺ |
| 479 | 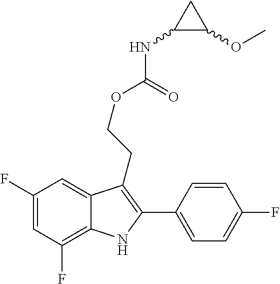 [SMILES: COC1CC1NC(=O)OCCc1c(-c2ccc(F)cc2)[nH]c2c(F)cc(F)cc12] | 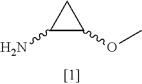 | ¹H NMR (300 MHz, Acetone-d₆) δ 10.76 (s, 1H), 7.93-7.66 (m, 2H), 7.39-7.14 (m, 3H), 6.84 (ddd, J = 11.6, 9.7, 2.2 Hz, 1H), 6.27 (s, 1H), 4.30 (t, J = 7.3 Hz, 2H), 3.35 (s, 3H), 3.25-2.95 (m, 3H), 2.50 (ddt, J = 8.0, 3.6, 1.7 Hz, 1H), 0.89 (ddd, J = 8.7, 6.6, 3.8 Hz, 1H), 0.73 (td, J = 6.8, 4.6 Hz, 1H); LCMS m/z 405.25 [M + H]⁺ |
| 480 | 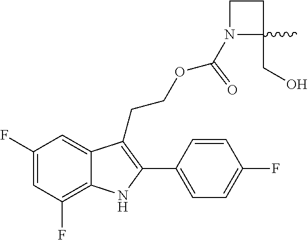 [SMILES: CC1(CO)CCN1C(=O)OCCc1c(-c2ccc(F)cc2)[nH]c2c(F)cc(F)cc12] | 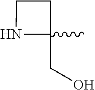 | LCMS m/z 419.29 [M + H]⁺ |
| 481 | 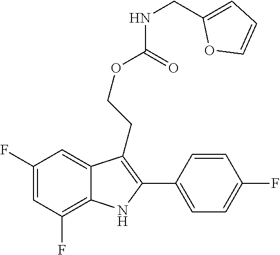 [SMILES: O=C(NCc1ccco1)OCCc1c(-c2ccc(F)cc2)[nH]c2c(F)cc(F)cc12] | 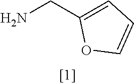 | ¹H NMR (300 MHz, Acetone-d₆) δ 10.78 (s, 1H), 7.80 (ddd, J = 8.6, 5.3, 2.4 Hz, 2H), 7.31 (ddt, J = 8.8, 5.2, 2.7 Hz, 3H), 6.84 (ddd, J = 11.5, 9.6, 2.2 Hz, 1H), 6.64 (s, 1H), 6.19-5.62 (m, 1H), 4.34 (t, J = 7.3 Hz, 2H), 3.50 (tt, J = 15.0, 5.4 Hz, 2H), 3.16 (t, J = 7.3 Hz, 2H); LCMS m/z 414.96 [M + H]⁺ |
| 482 | 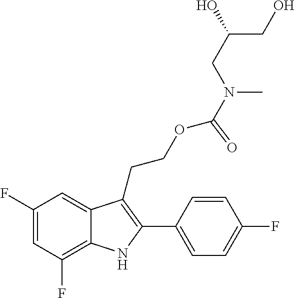 [SMILES: CN(C[C@H](O)CO)C(=O)OCCc1c(-c2ccc(F)cc2)[nH]c2c(F)cc(F)cc12] | 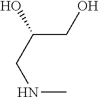 | LCMS m/z 423.36 [M + H]⁺ |
| 483 | 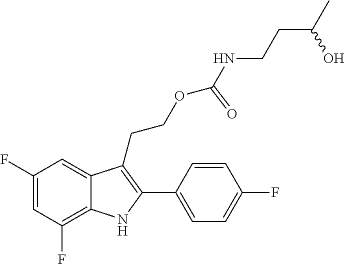 [SMILES: CC(O)CCNC(=O)OCCc1c(-c2ccc(F)cc2)[nH]c2c(F)cc(F)cc12] | 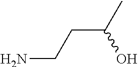 | LCMS m/z 407.37 [M + H]⁺ |
| 484 | 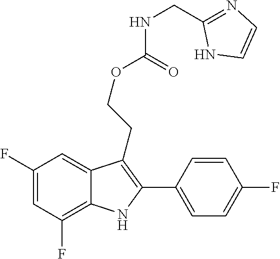 [SMILES: O=C(NCc1ncc[nH]1)OCCc1c(-c2ccc(F)cc2)[nH]c2c(F)cc(F)cc12] | 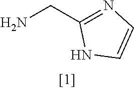 | LCMS m/z 415.16 [M + H]⁺ |
| 485 | 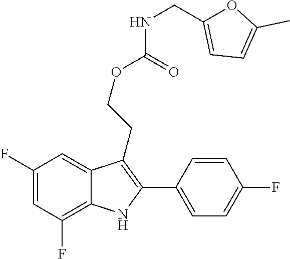 [SMILES: Cc1ccc(CNC(=O)OCCc2c(-c3ccc(F)cc3)[nH]c3c(F)cc(F)cc23)o1] | 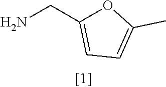 | LCMS m/z 429.23 [M + H]⁺ |
| 486 | 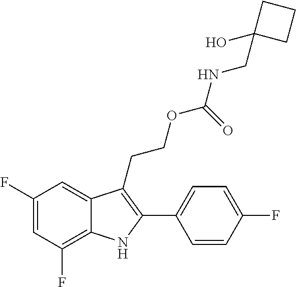 [SMILES: O=C(NCC1(O)CCC1)OCCc1c(-c2ccc(F)cc2)[nH]c2c(F)cc(F)cc12] | 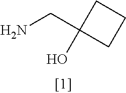 | ¹H NMR (300 MHz, Acetone-d₆) δ 10.76 (s, 1H), 8.03-7.54 (m, 2H), 7.41-7.16 (m, 3H), 6.84 (ddd, J = 11.5, 9.7, 2.2 Hz, 1H), 6.04 (s, 1H), 4.32 (t, J = 7.3 Hz, 2H), 3.74 (s, 1H), 3.39-2.94 (m, 4H), 2.00-1.77 (m, 4H), 1.84-1.15 (m, 2H); LCMS m/z 419.33 [M + H]⁺ |
| 487 | 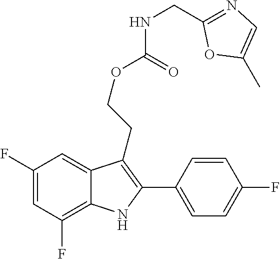 [SMILES: Cc1cnc(CNC(=O)OCCc2c(-c3ccc(F)cc3)[nH]c3c(F)cc(F)cc23)o1] | 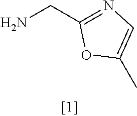 | ¹H NMR (300 MHz, Acetone-d₆) δ 10.76 (s, 1H), 7.95-7.59 (m, 2H), 7.30 (tq, J = 9.7, 3.1, 2.6 Hz, 3H), 6.84 (ddd, J = 11.1, 9.7, 2.2 Hz, 1H), 6.78-6.50 (m, 2H), 4.49-4.25 (m, 4H), 3.16 (t, J = 7.3 Hz, 2H), 2.27 (d, J = 1.2 Hz, 3H); LCMS m/z 430.14 [M + H]⁺ |
| 488 | 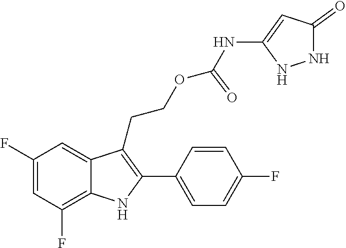 [SMILES: O=C(Nc1cc(=O)[nH][nH]1)OCCc1c(-c2ccc(F)cc2)[nH]c2c(F)cc(F)cc12] | 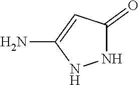 | LCMS m/z 416.91 [M + H]⁺ |
| 489 | 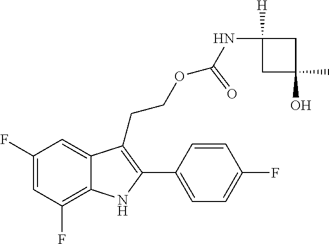 [SMILES: C[C@]1(O)C[C@@H](NC(=O)OCCc2c(-c3ccc(F)cc3)[nH]c3c(F)cc(F)cc23)C1] | 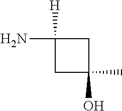 | LCMS m/z 419.03 [M + H]⁺ |
| 490 | 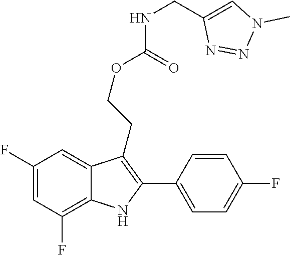 [SMILES: Cn1cc(CNC(=O)OCCc2c(-c3ccc(F)cc3)[nH]c3c(F)cc(F)cc23)nn1] | 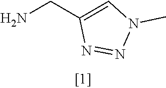 | LCMS m/z 430.14 [M + H]⁺ |
| 491 | 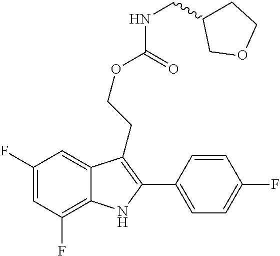 [SMILES: O=C(NCC1CCOC1)OCCc1c(-c2ccc(F)cc2)[nH]c2c(F)cc(F)cc12] | 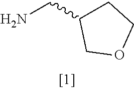 | ¹H NMR (300 MHz, Acetone-d₆) δ 10.76 (s, 1H), 7.96-7.60 (m, 2H), 7.45-7.15 (m, 3H), 6.84 (ddd, J = 11.1, 9.7, 2.2 Hz, 1H), 6.33 (s, 1H), 4.31 (t, J = 7.3 Hz, 2H), 3.86- 3.52 (m, 3H), 3.45 (dd, J = 8.6, 5.4 Hz, 1H), 3.13 (t, J = 7.1 Hz, 4H), 2.42 (dq, J = 13.6, 6.8 Hz, 1H), 1.95 (ddt, J = 10.8, 7.8, 4.0 Hz, 1H), 1.58 (dq, J = 13.2, 6.8 Hz, 1H); |
| | | | LCMS m/z 418.93 |
| | | | [M + H]⁺ |
| 492 | 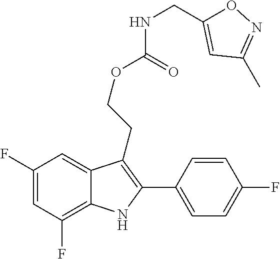 [SMILES: Cc1cc(CNC(=O)OCCc2c(-c3ccc(F)cc3)[nH]c3c(F)cc(F)cc23)on1] | 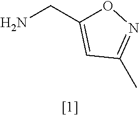 | LCMS m/z 430.11 [M + H]⁺ |
| 493 | 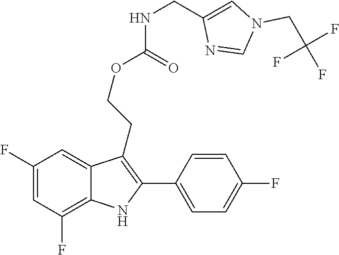 [SMILES: O=C(NCc1cn(CC(F)(F)F)cn1)OCCc1c(-c2ccc(F)cc2)[nH]c2c(F)cc(F)cc12] | 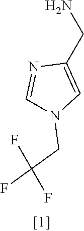 [SMILES: NCc1cn(CC(F)(F)F)cn1] | ¹H NMR (300 MHz, Acetone-d₆) δ 10.77 (s, 1H), 8.55 (s, 1H), 7.92-7.70 (m, 2H), 7.56 (s, 1H), 7.30 (ddd, J = 8.9, 5.3, 2.9 Hz, 4H), 6.83 (ddd, J = 11.1, 9.7, 2.2 Hz, 1H), 5.30 (q, J = 8.7 Hz, 2H), 4.58-4.11 (m, 4H), 3.13 (t, J = 7.3 Hz, 2H); LCMS m/z 497.11 [M + H]⁺ |
| 494 | 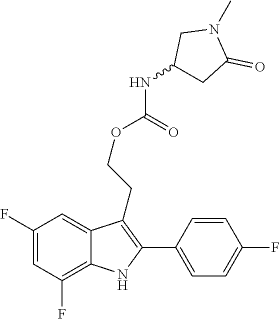 [SMILES: CN1CC(NC(=O)OCCc2c(-c3ccc(F)cc3)[nH]c3c(F)cc(F)cc23)CC1=O] | 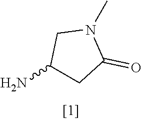 | LCMS m/z 432.16 [M + H]⁺ |
| 495 | 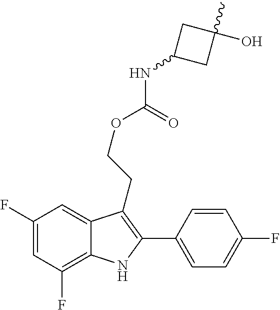 [SMILES: CC1(O)CC(NC(=O)OCCc2c(-c3ccc(F)cc3)[nH]c3c(F)cc(F)cc23)C1] | 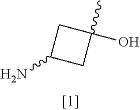 | ¹H NMR (300 MHz, Acetone-d₆) δ 10.76 (s, 1H), 8.05-7.60 (m, 2H), 7.60-7.13 (m, 3H), 6.84 (ddd, J = 11.1, 9.7, 2.2 Hz, 1H), 6.33 (d, J = 7.8 Hz, 1H), 4.28 (t, J = 7.3 Hz, 2H), 3.70 (p, J = 8.1 Hz, 3H), 3.12 (t, J = 7.3 Hz, 2H), 2.47- 2.16 (m, 2H), 2.05 (s, 1H), 1.31 (s, 3H); LCMS m/z 419.29 [M + H]⁺ |
| 496 | 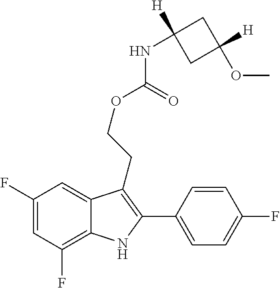 [SMILES: CO[C@H]1C[C@@H](NC(=O)OCCc2c(-c3ccc(F)cc3)[nH]c3c(F)cc(F)cc23)C1] | 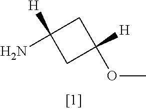 | LCMS m/z 418.93 [M + H]⁺ |
| 497 | 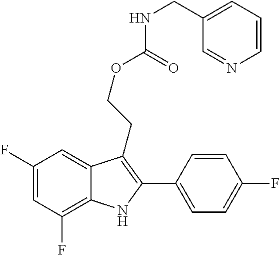 [SMILES: O=C(NCc1cccnc1)OCCc1c(-c2ccc(F)cc2)[nH]c2c(F)cc(F)cc12] | 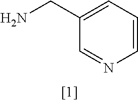 | ¹H NMR (300 MHz, Acetone-d₆) δ 10.84 (s, 1H), 8.75 (s, 2H), 8.23 (d, J = 8.0 Hz, 1H), 7.81 (td, J = 8.6, 5.4 Hz, 3H), 7.41- 7.18 (m, 3H), 7.18- 6.64 (m, 2H), 4.64- 4.08 (m, 4H), 3.16 (t, J = 7.2 Hz, 2H); LCMS m/z 426.13 [M + H]⁺ |
| 498 | 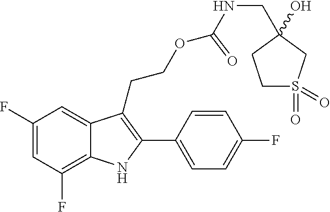 [SMILES: O=C(NCC1(O)CCS(=O)(=O)C1)OCCc1c(-c2ccc(F)cc2)[nH]c2c(F)cc(F)cc12] | 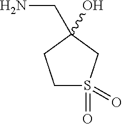 | LCMS m/z 483.21 [M + H]⁺ |
| 499 | 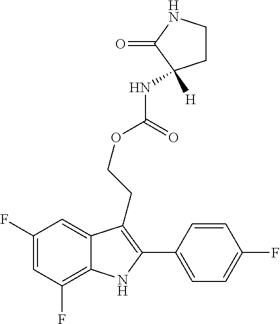 [SMILES: O=C(N[C@H]1CCNC1=O)OCCc1c(-c2ccc(F)cc2)[nH]c2c(F)cc(F)cc12] | 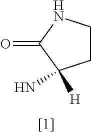 | LCMS m/z 418.54 [M + H]⁺ |
| 500 | 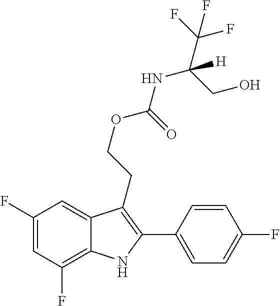 [SMILES: O=C(N[C@@H](CO)C(F)(F)F)OCCc1c(-c2ccc(F)cc2)[nH]c2c(F)cc(F)cc12] | 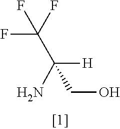 | LCMS m/z 446.85 [M + H]⁺ |
| 501 | 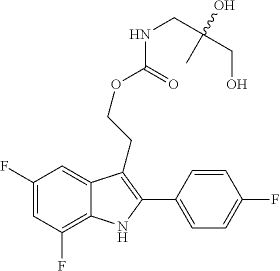 [SMILES: CC(O)(CO)CNC(=O)OCCc1c(-c2ccc(F)cc2)[nH]c2c(F)cc(F)cc12] | 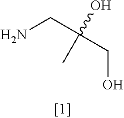 | ¹H NMR (300 MHz, Acetone-d₆) δ 10.76 (s, 1H), 7.96-7.54 (m, 2H), 7.49-7.08 (m, 3H), 6.84 (ddd, J = 11.1, 9.7, 2.2 Hz, 1H), 6.27 (s, 1H), 4.51- 4.23 (m, 2H), 3.61 (s, 2H), 3.31 (d, J = 2.3 Hz, 2H), 3.20-2.95 (m, 4H), 1.08 (s, 3H); LCMS m/z 423.04 [M + H]⁺ |
| 502 | 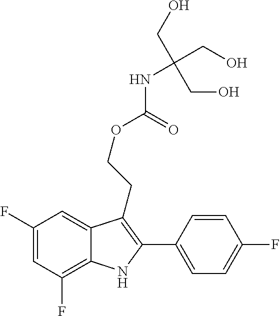 [SMILES: O=C(NC(CO)(CO)CO)OCCc1c(-c2ccc(F)cc2)[nH]c2c(F)cc(F)cc12] | 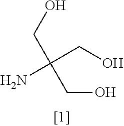 | ¹H NMR (300 MHz, Acetone-d₆) δ 10.77 (s, 1H), 8.01-7.56 (m, 2H), 7.31 (td, J = 9.2, 2.4 Hz, 3H), 6.84 (ddd, J = 11.1, 9.6, 2.2 Hz, 1H), 5.77 (s, 1H), 4.29 (t, J = 7.3 Hz, 2H), 3.71 (s, 6H), 3.15 (t, J = 7.3 Hz, 3H), 2.08-2.05 (m, 2H); LCMS m/z 439.36 [M + H]⁺ |

[1] Reaction was conducted at 90° C. and run overnight.

### Compound 503

### 3-(2-ethoxyethyl)-5,7-difluoro-2-(4-fluorophenyl)-1H-indole (503)

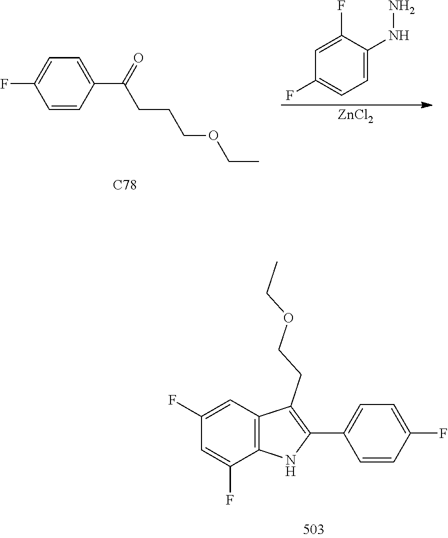

### Preparation of 3-(2-ethoxyethyl)-5,7-difluoro-2-(4-fluorophenyl)-1H-indole (503)

To a mixture of 4-ethoxy-1-(4-fluorophenyl)butan-1-one C78 (255.3 mg, 1.214 mmol) and (2,4-difluorophenyl)hydrazine hydrochloride (440 mg, 2.4 mmol) in acetic acid (5 mL) and toluene (5 mL) was added zinc chloride (750 mg, 5.5 mmol). The mixture was heated at 115° C. overnight and concentrated in vacuo. The residue was partitioned between EtOAc and water. The aqueous layer was extracted with EtOAc, and the combined organic layer was washed with brine, dried over sodium sulfate, filtered, and concentrated in vacuo. The residue was purified by silica gel chromatography (Gradient: 0-20% EtOAc in heptane) to give the product (97.9 mg, 24%). ¹H NMR (300 MHz, Chloroform-d) δ 8.10 (s, 1H), 7.66-7.58 (m, 2H), 7.22-7.15 (m, 2H), 7.10 (ddt, J=9.2, 2.2, 0.6 Hz, 1H), 6.74 (ddd, J=10.8, 9.5, 2.2 Hz, 1H), 3.68 (t, J=7.1 Hz, 2H), 3.48 (q, J=7.0 Hz, 2H), 3.05 (t, J=7.1 Hz, 2H), 1.19 (t, J=7.0 Hz, 3H). LCMS m/z 320.19 [M+H]⁺.

### Preparation S21

### 2-[5-(4-fluorophenyl)-5-oxo-pentyl]isoindoline-1,3-dione (S21)

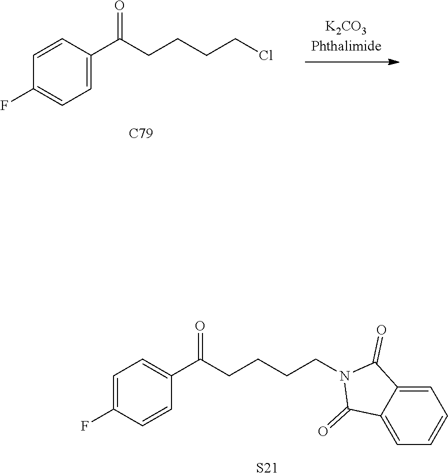 [SMILES: O=C(CCCCCl)c1ccc(F)cc1 ; "C79" ; O=C(CCCCN1C(=O)c2ccccc2C1=O)c1ccc(F)cc1 ; "S21"]

### Preparation of 2-[5-(4-fluorophenyl)-5-oxo-pentyl]isoindoline-1,3-dione (S21)

To a solution of 5-chloro-1-(4-fluorophenyl)pentan-1-one C79 (10 g, 46.58 mmol) and isoindoline-1,3-dione (6.90 g, 46.90 mmol) in DMF (100 mL) was added K₂CO₃ (6.90 g, 49.93 mmol). The reaction was heated to 70° C. for 16 hours. The reaction mixture was diluted with water and extracted with EtOAc (3×). The combined organic extracts were then washed with water and brine, dried over sodium sulfate, filtered, and concentrated in vacuo to afford crude product, 2-[5-(4-fluorophenyl)-5-oxo-pentyl]isoindoline-1,3-dione (S21) (15.2 g, 100%) as a residue which was used directly in the next reaction without further purification. Quantitative yield as assumed. LCMS m/z 326.15 [M+H]⁺.

### Compound 504

### 3-[5,7-difluoro-2-(4-fluorophenyl)-1H-indol-3-yl]propan-1-amine (504)

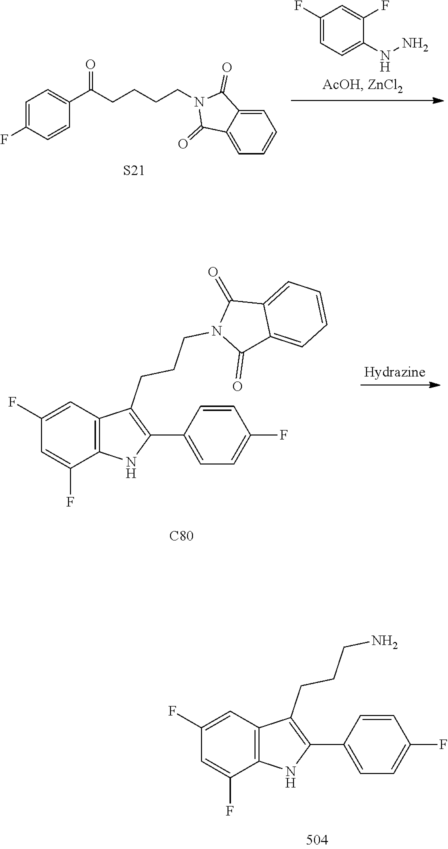 [SMILES: NNc1ccc(F)cc1F ; O=C(CCCCN1C(=O)c2ccccc2C1=O)c1ccc(F)cc1 ; "S21" ; O=C1c2ccccc2C(=O)N1CCCc1c(-c2ccc(F)cc2)[nH]c2c(F)cc(F)cc12 ; "C80" ; NCCCc1c(-c2ccc(F)cc2)[nH]c2c(F)cc(F)cc12]

### Step 1. Synthesis of 2-[3-[5,7-difluoro-2-(4-fluorophenyl)-1H-indol-3-yl]propyl]isoindoline-1,3-dione (C80)

A solution of 2-[5-(4-fluorophenyl)-5-oxo-pentyl]isoindoline-1,3-dione S21 (423 mg, 1.28 mmol), toluene (3 mL), AcOH (3 mL), dichlorozinc (900 mg, 6.6 mmol), and (2,4-difluorophenyl)hydrazine (737 mg, 5.11 mmol) was heated to 110° C. with stirring for 6 hours before cooling to room temperature. The reaction was quenched into aq. saturated sodium bicarbonate (150 mL) and extracted with ethyl acetate (2×100 mL). The combined organics were washed with brine, dried with MgSO4, filtered and concentrated in vacuo. Purification by silica gel chromatography (Gradient: 0-25% EtOAc/heptanes) afforded the product 2-[3-[5,7-difluoro-2-(4-fluorophenyl)-1H-indol-3-yl]propyl]isoindoline-1,3-dione (C80) (510 mg, 40%) as a yellow solid. LCMS m/z 435.31 [M+H]⁺.

### Step 2. Synthesis of 3-[5,7-difluoro-2-(4-fluorophenyl)-1H-indol-3-yl]propan-1-amine (504)

To a solution of 2-[3-[5,7-difluoro-2-(4-fluorophenyl)-1H-indol-3-yl]propyl]iso-indoline-1,3-dione C80 (498 mg, 1.146 mmol) in ethanol (11 mL) was added hydrazine monohydrate (800 μL, 16.32 mmol). The reaction was heated to 80° C. with stirring for 8 hours before cooling to room temperature. The reaction was diluted with additional ethanol and filtered through a bed of Celite®. The filtrate was concentrated in vacuo to afford 3-[5,7-difluoro-2-(4-fluorophenyl)-1H-indol-3-yl]propan-1-amine (474 mg, 100%) as a light orange solid. LCMS m/z 305.24 [M+H]⁺.

### Alternative Preparation of 504

### 3-[5,7-difluoro-2-(4-fluorophenyl)-1H-indol-3-yl]propan-1-amine (504)

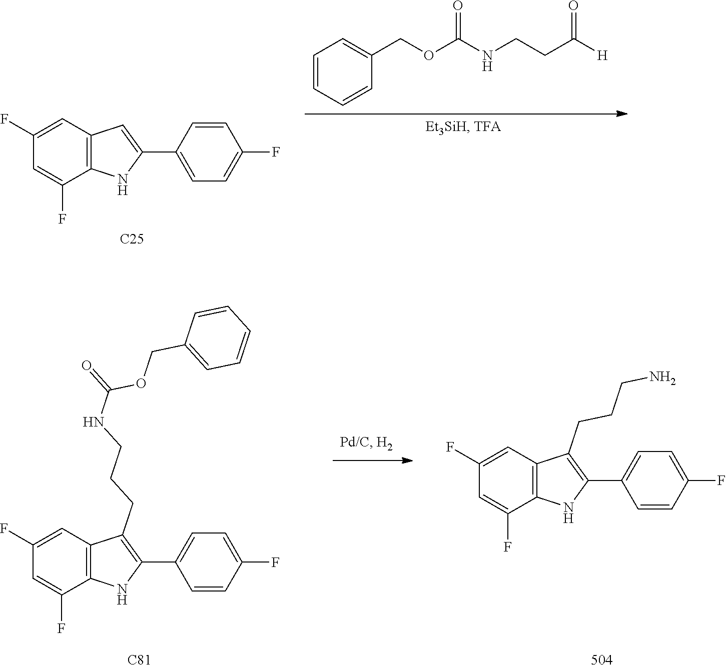

### Step 1. Synthesis of benzyl N-[3-[5,7-difluoro-2-(4-fluorophenyl)-1H-indol-3-yl]propyl]-carbamate (C81)

To a solution of 5,7-difluoro-2-(4-fluorophenyl)-1H-indole C25 (100 mg, 0.39 mmol) and benzyl N-(3-oxopropyl)carbamate (93 mg, 0.45 mmol) in dichloromethane (6 mL) under nitrogen was added triethylsilane (188 μL, 1.177 mmol) and TFA (90 μL, 1.168 mmol). The reaction was heated to 40° C. overnight, at which time it was cooled to room temperature and partitioned between dichloromethane and aq. saturated sodium bicarbonate. The organics were collected through a phase separator and concentrated in vacuo. Purification by silica gel chromatography (Gradient: 0-100% EtOAc in Heptanes) afforded benzyl N-[3-[5,7-difluoro-2-(4-fluorophenyl)-1H-indol-3-yl]propyl]carbamate (C81) (95.7 mg, 54%). ¹H NMR (300 MHz, Chloroform-d) δ 8.12 (s, 1H), 7.53-7.42 (m, 2H), 7.34 (q, J=2.7, 1.6 Hz, 5H), 7.21-7.10 (m, 2H), 7.02 (dd, J=9.1, 2.1 Hz, 1H), 6.74 (ddd, J=10.8, 9.4, 2.2 Hz, 1H), 5.07 (s, 2H), 4.63 (s, 1H), 3.18 (q, J=6.7 Hz, 2H), 2.87-2.74 (m, 2H), 1.84 (p, J=7.3 Hz, 2H). LCMS m/z 439.33 [M+H]⁺.

### Step 2. Synthesis of 3-[5,7-difluoro-2-(4-fluorophenyl)-1H-indol-3-yl]propan-1-amine (504)

A solution of benzyl N-[3-[5,7-difluoro-2-(4-fluorophenyl)-1H-indol-3-yl]propyl]carbamate C81 (90 mg, 0.197 mmol) and palladium on carbon (97 mg of 2.2% w/w, 0.02 mmol) in ethanol (3 mL) was purged and evacuated with nitrogen and allowed to stir at room temperature for 3 hours under a hydrogen balloon. The reaction was filtered and concentrated in vacuo. Purification by silica gel chromatography (Gradient: 0-100% EtOAc in heptanes) afforded 3-[5,7-difluoro-2-(4-fluorophenyl)-1H-indol-3-yl]propan-1-amine (40.5 mg, 65%) ¹H NMR (300 MHz, Chloroform-d) δ 8.19 (s, 1H), 7.51 (ddd, J=8.0, 5.1, 2.3 Hz, 2H), 7.22-7.12 (m, 2H), 7.06 (dd, J=9.2, 2.2 Hz, 1H), 6.73 (ddd, J=11.5, 9.5, 2.2 Hz, 1H), 2.86-2.78 (m, 2H), 2.72 (t, J=7.1 Hz, 2H), 1.84-1.73 (m, 2H). LCMS m/z 305.07 [M+H]⁺.

### Compound 505

### N-[3-[5,7-difluoro-2-(4-fluorophenyl)-1H-indol-3-yl]propyl]acetamide (505)

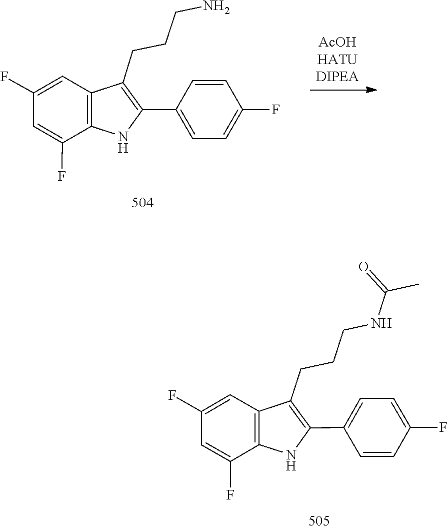 [SMILES: NCCCc1c(-c2ccc(F)cc2)[nH]c2c(F)cc(F)cc12 ; CC(=O)NCCCc1c(-c2ccc(F)cc2)[nH]c2c(F)cc(F)cc12]

### Preparation of N-[3-[5,7-difluoro-2-(4-fluorophenyl)-1H-indol-3-yl]propyl]acetamide (505)

To a stirred solution of 3-[5,7-difluoro-2-(4-fluorophenyl)-1H-indol-3-yl]propan-1-amine 504 (780 mg, 0.0018 mol) in DMF (12 mL) were added DIPEA (1.4692 g, 2 mL, 0.0113 mol), AcOH (261.36 mg, 0.25 mL, 0.0043 mol) and HATU (1.1 g, 0.0028 mol) at room temperature. The reaction mixture was heated to 50° C. for 3 hours at which time the reaction mixture was poured into ice cold water (100 mL) and extracted with EtOAc (3×100 mL). The organic layer was washed with brine solution (80 mL), dried over sodium sulfate, filtered, concentrated in vacuo. Purification by reversed-phase HPLC (Method: C18 Luna column (25×150 mm, 10 micron). Gradient: MeOH in H₂O with 10 mM ammonium bicarbonate) afforded an off-white solid which was washed with n-pentane (10 mL) and dried under vacuum to yield N-[3-[5,7-difluoro-2-(4-fluorophenyl)-1H-indol-3-yl]propyl]acetamide (326 mg, 52%). ¹H NMR (400 MHz, DMSO-d₆) δ 11.64 (brs, 1H), 7.85-7.82 (m, 1H), 7.67-7.63 (m, 2H), 7.38-7.33 (m, 2H), 7.25-7.22 (m, 1H), 6.99-6.93 (m, 1H), 3.18-3.04 (m, 2H), 2.76-2.72 (m, 2H), 1.78 (s, 3H), 1.74-1.66 (m, 2H). ¹⁹F NMR (376.22 MHz, DMSO-d₆) δ −113.76, −122.33, −129.31; LCMS m/z 347.1 [M+H]⁺.

### Preparation S22

### 3-[5-fluoro-2-(4-fluorophenyl)-1H-indol-3-yl]propan-1-amine (S22)

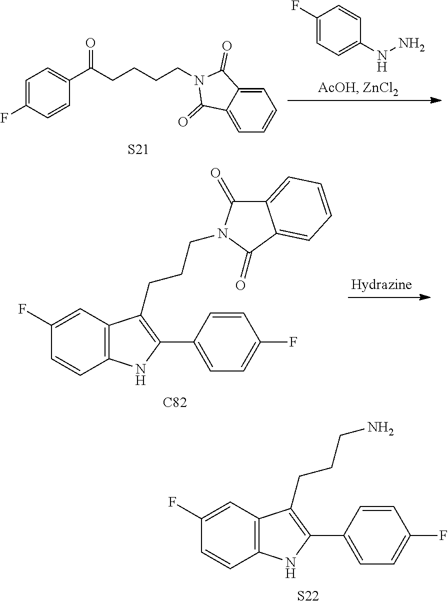

### Step 1. Synthesis of 2-[3-[5-fluoro-2-(4-fluorophenyl)-1H-indol-3-yl]propyl]isoindoline-1,3-dione (C82)

To a solution of dichlorozinc (1.9 g, 14 mmol) and 2-[5-(4-fluorophenyl)-5-oxo-pentyl]isoindoline-1,3-dione S21 (2.2 g, 6.8 mmol) in AcOH (30 mL) was added (4-fluorophenyl)hydrazine (Hydrochloride salt) (1.5 g, 9.226 mmol). The reaction was heated to 70° C. for 4 hours. An extra portion of hydrazine (1 equiv.) was added to the mixture, and the mixture was heated at 70° C. for 2 more hours. The reaction mixture was filtered to remove ZnCl₂ and the filtrate was concentrated in vacuo. The residue was dissolved in EtOAc, washed with water (2×), brine (2×) dried, and concentrated to afford 2-[3-[5-fluoro-2-(4-fluorophenyl)-1H-indol-3-yl]propyl]isoindoline-1,3-dione (2.85 g, 100%) as an orange solid. The material was taken forward without further purification and quantitative yield was assumed. LCMS m/z 417.27 [M+H]⁺

### Step 2. Synthesis of 3-[5-fluoro-2-(4-fluorophenyl)-1H-indol-3-yl]propan-1-amine (S22)

To a solution of 2-[3-[5-fluoro-2-(4-fluorophenyl)-1H-indol-3-yl]propyl]isoindoline-1,3-dione C82 (2.82 g, 6.77 mmol) in EtOH (30 mL) was added hydrazine (30 mL of 1 M, 30 mmol). The reaction was heated to 50° C. and stirred for 2 hours. Additional hydrazine (4 equiv.) was added and the mixture was heated for 3 more hours. The volatiles were removed under reduced pressure and the solid residue was taken up in EtOH and filtered. The filtrate was concentrated in vacuo to afford 3-[5-fluoro-2-(4-fluorophenyl)-1H-indol-3-yl]propan-1-amine (S22) (1.82 g, 94%) which was directly used in the next step. LCMS m/z 287.2 [M+H]⁺.

### Compounds 506 and 507

Compounds 506 and 507 were prepared in one step according to the procedure as described for 505 using the appropriate amine. Acids were obtained from commercial sources.

TABLE 14
Structure, acid starting material, and physicochemical data

| | | | ¹H NMR; LCMS m/z |
|---|---|---|---|
| Compound | Product | Acid | [M + H]⁺ |
| 506 | 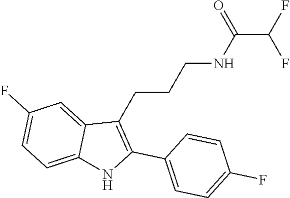 [SMILES: O=C(NCCCc1c(-c2ccc(F)cc2)[nH]c2ccc(F)cc12)C(F)F] | 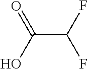 | LCMS m/z 365.13 [M + H]⁺ |
| 507 | 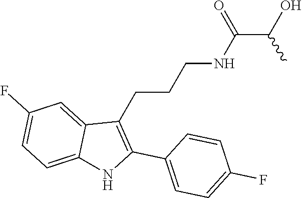 [SMILES: CC(O)C(=O)NCCCc1c(-c2ccc(F)cc2)[nH]c2ccc(F)cc12] | 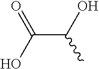 | LCMS m/z 359.59 [M + H]⁺ |

### Compound 508

### N-[3-[5,7-difluoro-2-(4-fluorophenyl)-1H-indol-3-yl]propyl]acetamide (508)

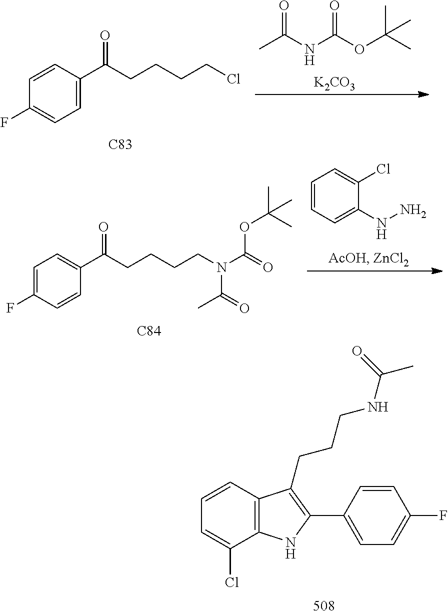

### Step 1. Synthesis of tert-butyl N-acetyl-N-[5-(4-fluorophenyl)-5-oxo-pentyl]carbamate (C84)

A solution of 5-chloro-1-(4-fluorophenyl)pentan-1-one C83 (5.0 g, 23 mmol), tert-butyl N-acetylcarbamate (3.7 g, 23 mmol) and potassium carbonate (4.8 g, 35 mmol) in DMF (50 mL) was heated overnight at 80° C. The reaction mixture was filtered, and the filtrate partitioned with water and extracted with ethyl acetate. The organic layer was washed with brine, dried over sodium sulfate and concentrated in vacuo. Purification by silica gel chromatography (Gradient: 0-20% EtOAc in Heptanes) afforded tert-butyl N-acetyl-N-[5-(4-fluorophenyl)-5-oxo-pentyl]carbamate (5.5 g, 21%). LCMS m/z 337.16 [M+H]⁺.

### Step 2. Synthesis of N-[3-[7-chloro-2-(4-fluorophenyl)-1H-indol-3-yl]propyl]acetamide (508)

A solution of (2-chlorophenyl)hydrazine (200 mg, 1.403 mmol), tert-butyl N-acetyl-N-[5-(4-fluorophenyl)-5-oxo-pentyl]carbamate C84 (473.4 mg, 1.403 mmol) and dichlorozinc (478.2 mg, 325.4 μL, 3.508 mmol) in toluene (5 mL) and AcOH (5 mL) were heated to 120° C. overnight. The reaction mixture was cooled to room temperature, filtered, and concentrated to dryness. The residue was brought up in water and extracted with ethyl acetate. The organic layer was concentrated in vacuo. Purification by reversed-phase HPLC (Method: C18 Waters Sunfire column (30×150 mm, 5 micron). Gradient: MeCN in H₂O with 0.1% trifluoroacetic acid) afforded N-[3-[7-chloro-2-(4-fluorophenyl)-1H-indol-3-yl]propyl]acetamide (508) (16.9 mg) as the trifluoroacetate salt. ¹H NMR (300 MHz, Methanol-d₄) δ 7.72-7.44 (m, 3H), 7.27-6.91 (m, 4H), 3.15 (t, J=7.0 Hz, 2H), 2.84 (t, J=7.9 Hz, 2H), 1.86 (d, J=10.4 Hz, 5H). LCMS m/z 345.17 [M+H]⁺.

### Compound 509

### N-[2-[[5,7-difluoro-2-(4-fluorophenyl)-1H-indol-3-yl]sulfanyl]ethyl]acetamide (509)

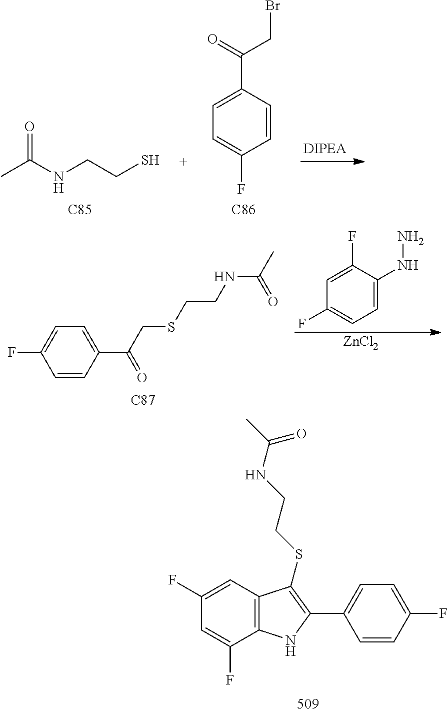

### Step 1. Synthesis of N-[2-[2-(4-fluorophenyl)-2-oxo-ethyl]sulfanylethyl]acetamide (C87)

A solution of N-(2-sulfanylethyl)acetamide C85 (250 mg, 2.098 mmol) and 2-bromo-1-(4-fluorophenyl)ethanone C86 (456 mg, 2.10 mmol) in DMF (4 mL) was treated with DIPEA (406 μL, 2.331 mmol) at 22° C. The reaction was stirred for 2 hours. Water was added, and the mixture was extracted with EtOAc. The organic layer was washed with brine, dried over Na₂SO₄ and evaporated. Purification by silica gel chromatography (Gradient: 0-40% EtOAc in heptane) yielded the product: N-[2-[2-(4-fluorophenyl)-2-oxo-ethyl]sulfanylethyl]acetamide (305 mg, 54%). ¹H NMR (300 MHz, Chloroform-d) δ 8.05-7.97 (m, 2H), 7.20-7.12 (m, 2H), 6.00 (s, 1H), 3.84 (s, 2H), 3.47 (q, J=6.0 Hz, 2H), 2.73 (dd, J=6.8, 5.7 Hz, 2H), 1.99 (s, 3H). LCMS m/z 256.18 [M+H]⁺.

### Step 2. Synthesis of N-[2-[[5,7-difluoro-2-(4-fluorophenyl)-1H-indol-3-yl]sulfanyl]ethyl]acetamide (509)

A solution of N-[2-[2-(4-fluorophenyl)-2-oxo-ethyl]sulfanylethyl]acetamide C87 (305 mg, 1.141 mmol), (2,4-difluorophenyl)hydrazine (Hydrochloride salt) (413 mg, 2.287 mmol), acetic acid (3 mL) in toluene (3 mL) was treated with zinc chloride (702 mg, 5.150 mmol). The mixture was stirred at 115° C. overnight and then concentrated in vacuo. Purification by reversed-phase HPLC (Method: C18 column. Gradient: MeCN in H₂O with 0.1% trifluoroacetic acid) and further purification by silica gel chromatography (Gradient: 0-100% EtOAc in heptane) yielded the product. N-[2-[[5,7-difluoro-2-(4-fluorophenyl)-1H-indol-3-yl]sulfanyl]ethyl]acetamide (16.9 mg, 4%). ¹H NMR (300 MHz, Chloroform-d) δ 8.67 (s, 1H), 7.85-7.78 (m, 2H), 7.26-7.18 (m, 3H), 6.81 (ddd, J=10.6, 9.3, 2.2 Hz, 1H), 5.32 (s, 1H), 3.05 (q, J=6.0 Hz, 2H), 2.69 (dd, J=6.7, 5.5 Hz, 2H), 1.72 (s, 3H). LCMS m/z 365.1 [M+H]⁺.

### Compound 510

### N-[2-[[2-(4-fluorophenyl)-1H-indol-3-yl]sulfanyl]ethyl]acetamide (510)

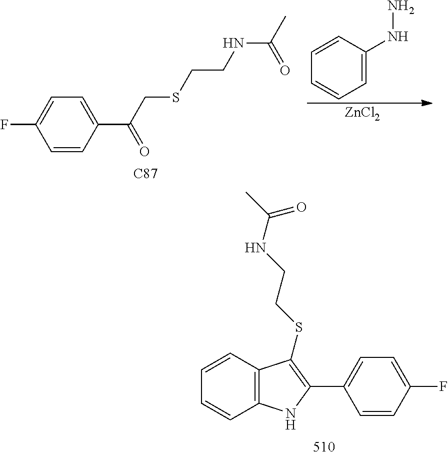

### Preparation of N-[2-[[2-(4-fluorophenyl)-1H-indol-3-yl]sulfanyl]ethyl]acetamide (510)

Phenylhydrazine (2.5 mL, 25.38 mmol) was added to a solution of zinc chloride (2.4 g, 17.61 mmol) and N-[2-[2-(4-fluorophenyl)-2-oxo-ethyl]sulfanylethyl]acetamide C87 (5.3 g, 20.76 mmol) in AcOH (80 mL). The reaction was heated to 70° C. for 4 hours and was concentrated in vacuo. The residue was dissolved in EtOAc and washed with water, brine and dried and concentrated under reduced pressure. The residue was then purified by silica gel chromatography (Gradient: 0-100% EtOAc in hexanes) to afford the title compound as a beige solid N-[2-[[2-(4-fluorophenyl)-1H-indol-3-yl]sulfanyl]ethyl]acetamide (3.7 g, 54%). ¹H NMR (400 MHz, DMSO-d₆) δ 11.81 (s, 1H), 8.06-7.97 (m, 2H), 7.82 (t, J=5.5 Hz, 1H), 7.67 (dt, J=7.5, 0.9 Hz, 1H), 7.43 (dt, J=8.0, 1.0 Hz, 1H), 7.38 (t, J=9.0 Hz, 2H), 7.19 (ddd, J=8.1, 7.0, 1.3 Hz, 1H), 7.13 (ddd, J=8.0, 7.0, 1.1 Hz, 1H), 3.01 (dt, J=8.0, 6.0 Hz, 2H), 2.70-2.63 (m, 2H), 1.69 (s, 3H). LCMS m/z 329.15 [M+H]⁺.

### Compound 511

### 4-[5,7-difluoro-2-(4-fluorophenyl)-1H-indol-3-yl]butan-1-amine (511)

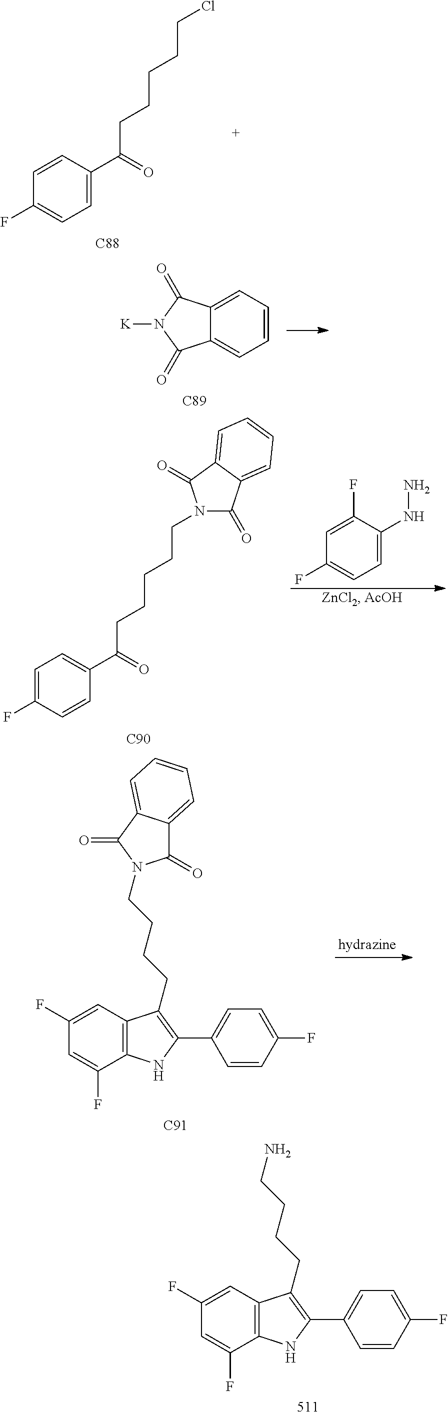

### Step 1. Synthesis of 2-[6-(4-fluorophenyl)-6-oxo-hexyl]isoindoline-1,3-dione (C90)

A solution of 6-chloro-1-(4-fluorophenyl)hexan-1-one C88 (1 g, 4.373 mmol) in DMF (45 mL) was treated with (1,3-dioxoisoindolin-2-yl)potassium (990 mg, 5.345 mmol) then heated/stirred at 80° C. overnight. The reaction was quenched with water causing precipitation of product as white solid. The product was collected by vacuum filtration, washed with water, dried under vacuum to afford 2-[6-(4-fluorophenyl)-6-oxo-hexyl]isoindoline-1,3-dione C90 (1.372 g, 88%)¹H NMR (300 MHz, Chloroform-d) δ 8.03-7.95 (m, 2H), 7.86 (dd, J=5.4, 3.1 Hz, 2H), 7.78-7.69 (m, 2H), 7.18-7.09 (m, 2H), 3.73 (t, J=7.2 Hz, 2H), 3.00-2.92 (m, 2H), 1.80 (dq, J=14.2, 7.3 Hz, 4H), 1.47 (td, J=8.5, 7.7, 3.1 Hz, 2H). LCMS m/z 340.05 [M+H]⁺.

### Step 2. Synthesis of 2-[4-[5,7-difluoro-2-(4-fluorophenyl)-1H-indol-3-yl]butyl]isoindoline-1,3-dione (C91)

A mixture of 2-[6-(4-fluorophenyl)-6-oxo-hexyl]isoindoline-1,3-dione C90 (660 mg, 1.835 mmol), (2,4-difluorophenyl)hydrazine (Hydrochloride salt) (665 mg, 3.683 mmol), and zinc chloride (1.2 g, 8.803 mmol) in toluene (5 mL) and acetic acid (5 mL) was heated and stirred at 110° C. overnight. The reaction was concentrated in vacuo then partitioned between ethyl acetate/water. The combined organics were washed with brine, dried over anhydrous sodium sulfate, filtered, and concentrated in vacuo followed by purification by silica gel chromatography (Gradient: 0-20% EtOAc in heptane) yielded the product 2-[4-[5,7-difluoro-2-(4-fluorophenyl)-1H-indol-3-yl]butyl]isoindoline-1,3-dione (C91) (450 mg, 48%). ¹H NMR (300 MHz, Chloroform-d) δ 8.04 (s, 1H), 7.85-7.80 (m, 2H), 7.71 (dt, J=5.2, 3.5 Hz, 2H), 7.53-7.46 (m, 2H), 7.20-7.13 (m, 2H), 7.03 (dd, J=9.2, 2.2 Hz, 1H), 6.70 (ddd, J=10.8, 9.5, 2.2 Hz, 1H), 3.69-3.62 (m, 2H), 2.82 (t, J=7.1 Hz, 2H), 1.69 (d, J=7.1 Hz, 4H). LCMS m/z 449.27 [M+H]⁺.

### Step 3. Synthesis of 4-[5,7-difluoro-2-(4-fluorophenyl)-1H-indol-3-yl]butan-1-amine (511)

A solution of 2-[4-[5,7-difluoro-2-(4-fluorophenyl)-1H-indol-3-yl]butyl]isoindoline-1,3-dione C91 (0.87 g, 1.843 mmol) in ethanol (18 mL) was treated with hydrazine (5.5 mL of 1 M, 5.500 mmol). The resulting solution was heated at reflux for 4 hours then cooled to ambient temperature and concentrated in vacuo. The residue was purified by reversed-phase HPLC (Method: C18 Waters Sunfire column (30×150 mm, 5 micron). Gradient: MeCN in H₂O with 0.1% trifluoroacetic acid) to afford 4-[5,7-difluoro-2-(4-fluorophenyl)-1H-indol-3-yl]butan-1-amine (Trifluoroacetate salt) (228.4 mg, 27%). ¹H NMR (300 MHz, DMSO-d₆) δ 11.69 (s, 1H), 7.70-7.64 (m, 2H), 7.59 (s, 2H), 7.41-7.33 (m, 2H), 7.25 (dd, J=9.6, 2.2 Hz, 1H), 6.98 (ddd, J=11.2, 9.8, 2.2 Hz, 1H), 2.83-2.70 (m, 4H), 1.56 (td, J=15.5, 14.7, 7.7 Hz, 4H). LCMS m/z 319.07 [M+H]⁺.

### Compound 512

### N-[4-[5,7-difluoro-2-(4-fluorophenyl)-1H-indol-3-yl]butyl]acetamide (512)

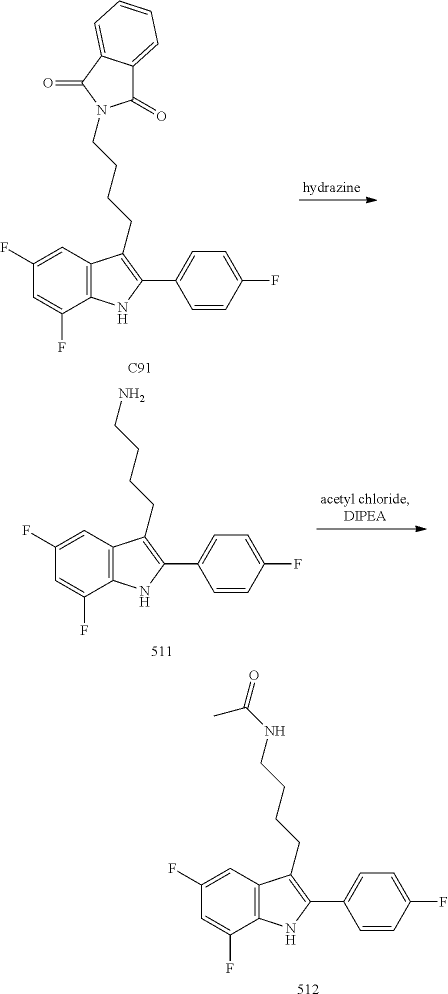 [SMILES: O=C1c2ccccc2C(=O)N1CCCCc1c(-c2ccc(F)cc2)[nH]c2c(F)cc(F)cc12 ; "C91" ; NCCCCc1c(-c2ccc(F)cc2)[nH]c2c(F)cc(F)cc12 ; "511" ; CC(=O)NCCCCc1c(-c2ccc(F)cc2)[nH]c2c(F)cc(F)cc12 ; "512"]

### Step 1. Synthesis of 4-[5,7-difluoro-2-(4-fluorophenyl)-1H-indol-3-yl]butan-1-amine (511)

A solution of 2-[4-[5,7-difluoro-2-(4-fluorophenyl)-1H-indol-3-yl]butyl]isoindoline-1,3-dione C91 (450 mg, 0.8840 mmol) in ethanol (9 mL) was treated with hydrazine (2.7 mL of 1 M, 2.700 mmol). The resulting mixture was heated at 60° C. for approximately 3-4 hours. The reaction was concentrated in vacuo then suspended in additional ethanol and filtered. The filtrated was concentrated in vacuo then dried under high vacuum to afford 4-[5,7-difluoro-2-(4-fluorophenyl)-1H-indol-3-yl]butan-1-amine which was used without further purification.

### Step 2. Synthesis of 4-[5,7-difluoro-2-(4-fluorophenyl)-1H-indol-3-yl]butan-1-amine (512)

The product from the above reaction was dissolved in dichloromethane (9 mL) and treated with acetyl chloride (63 μL, 0.8860 mmol) followed by DIPEA (308 μL, 1.768 mmol). The resulting solution was stirred at room temperature overnight, followed by concentration in vacuo and purified by silica gel chromatography (Gradient: 10-100% EtOAc in heptane) afforded the product N-[4-[5,7-difluoro-2-(4-fluorophenyl)-1H-indol-3-yl]butyl]acetamide (51.7 mg, 14% over 2 steps). ¹H NMR (300 MHz, Chloroform-d) δ 8.11 (s, 1H), 7.53-7.46 (m, 2H), 7.19 (td, J=8.9, 2.4 Hz, 2H), 7.03 (dd, J=9.1, 2.2 Hz, 1H), 6.74 (ddd, J=10.7, 9.4, 2.1 Hz, 1H), 5.35 (s, 1H), 3.26-3.18 (m, 2H), 2.80 (t, J=7.5 Hz, 2H), 1.94 (s, 3H), 1.66 (t, J=8.0 Hz, 2H), 1.50 (dt, J=8.2, 6.7 Hz, 2H). LCMS m/z 361.24 [M+H]⁺.

### Compound 513 and Compound 514

### 3-[5,7-difluoro-2-(4-fluorophenyl)-1H-indol-3-yl]-2-methyl-N-[(1S)-2, 2,2-trifluoro-1-(hydroxymethyl)ethyl]propanamide (513) 3-[5,7-difluoro-2-(4-fluorophenyl)-1H-indol-3-yl]-2-methyl-N-[(1S)-2, 2,2-trifluoro-1-(hydroxymethyl)ethyl]propenamide (514)

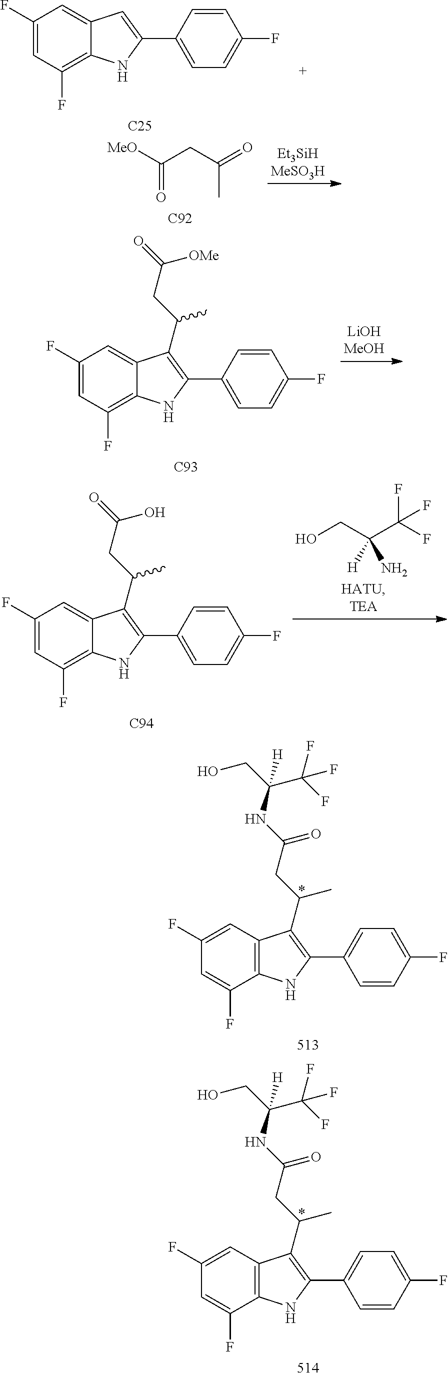

### Step 1: Synthesis of methyl 3-[5,7-difluoro-2-(4-fluorophenyl)-1H-indol-3-yl]butanoate (C93)

To 5,7-difluoro-2-(4-fluorophenyl)-1H-indole (505 mg, 2.043 mmol) and methyl 3-oxobutanoate (353 μL, 3.271 mmol) in DCE (8 mL) at 70° C. was added, MsOH (265 μL, 4.084 mmol) and triethylsilane (980 μL, 6.136 mmol). The reaction was heated at 70° C. for 1 hour. Additional methyl 3-oxobutanoate (353 μL, 3.271 mmol), MsOH (265 μL, 4.084 mmol), triethylsilane (980 μL, 6.136 mmol) were added and the mixture allowed to stir for overnight at 70° C. Water (50 mL) was added and the mixture was extracted with dichloromethane (3×). The mixture was concentrated and purified by silica gel chromatography (Gradient: 0 to 100% EtOAc in hexanes) to afford the product. methyl 3-[5,7-difluoro-2-(4-fluorophenyl)-1H-indol-3-yl]butanoate (144 mg, 20%). ¹H NMR (300 MHz, Chloroform-d) δ 8.07 (s, 1H), 7.65-7.48 (m, 2H), 7.21 (dtd, J=9.3, 6.7, 2.2 Hz, 3H), 6.76 (ddd, J=10.8, 9.4, 2.2 Hz, 1H), 3.67 (p, J=7.3 Hz, 1H), 3.58 (s, 3H), 2.81 (dd, J=7.7, 1.5 Hz, 2H), 1.44 (d, J=7.1 Hz, 3H). LCMS m/z 348.23 [M+H]⁺.

### Step 2: Synthesis of 3-[5,7-difluoro-2-(4-fluorophenyl)-1H-indol-3-yl]butanoic acid (C94)

A solution of methyl 3-[5,7-difluoro-2-(4-fluorophenyl)-1H-indol-3-yl]butanoate (391.8 mg, 1.072 mmol) in THF (5 mL)/MeOH (5 mL)/Water (2 mL) was treated with LiOH (55 mg, 2.297 mmol), and the resulting reaction stirred for approximately 2 h at room temperature. The reaction was concentrated in vacuo then the aqueous layer was acidified to pH 3 with 1N HCl. The aqueous layer was then extracted with ethyl acetate 3×, the combined organics were washed with brine, dried over anhydrous sodium sulfate, filtered, and concentrated in vacuo to afford 3-[5,7-difluoro-2-(4-fluorophenyl)-1H-indol-3-yl]butanoic acid (350.4 mg, 93%). ¹H NMR (300 MHz, Chloroform-d) δ 8.03 (s, 1H), 7.49-7.40 (m, 2H), 7.20-7.11 (m, 3H), 6.74 (ddd, J=10.7, 9.4, 2.1 Hz, 1H), 3.58 (p, J=7.3 Hz, 1H), 2.88-2.72 (m, 2H), 1.44 (d, J=7.1 Hz, 3H). LCMS m/z 334.1 [M+1]⁺.

### Step 3. Preparation of 3-[5,7-difluoro-2-(4-fluorophenyl)-1H-indol-3-yl]-2-methyl-N-[(1S)-2,2,2-trifluoro-1-(hydroxymethyl)ethyl]propanamide (513) and 3-[5,7-difluoro-2-(4-fluorophenyl)-1H-indol-3-yl]-2-methyl-N-[(1S)-2,2,2-trifluoro-1-(hydroxymethyl)ethyl]propanamide (514)

To 3-[5,7-difluoro-2-(4-fluorophenyl)-1H-indol-3-yl]-2-methyl-propanoic acid C94 (18 mg, 0.05400 mmol), (2S)-2-amino-3,3,3-trifluoro-propan-1-ol (Hydrochloride salt) (13 mg, 0.07853 mmol) and HATU (40 mg, 0.1052 mmol) in DMSO (1 mL) was added TEA (40 μL, 0.2870 mmol). The reaction was stirred at room temperature for 12 hours. Purification by reversed-phase HPLC (Method: C18 Waters Sunfire column (30×150 mm, 5 micron). Gradient: MeCN in H₂O with 0.1% trifluoroacetic acid) yielded a pair of diastereomers. Compound 513 was the first eluting isomer and compound 514 was the second eluting isomer. 3-[5,7-difluoro-2-(4-fluorophenyl)-1H-indol-3-yl]-2-methyl-N-[(1S)-2,2,2-trifluoro-1-(hydroxymethyl)ethyl]propanamide 513 (5 mg, 20%)¹H NMR (300 MHz, Methanol-d₄) δ 8.29 (d, J=9.3 Hz, 1H), 7.72-7.59 (m, 2H), 7.34-7.13 (m, 3H), 6.72 (ddd, J=11.7, 9.6, 2.2 Hz, 1H), 4.61-4.42 (m, OH), 3.50 (t, J=6.0 Hz, 2H), 3.15 (dd, J=14.0, 6.9 Hz, 1H), 2.86 (ddd, J=26.7, 14.1, 7.3 Hz, 2H), 1.04 (d, J=6.7 Hz, 3H). LCMS m/z 445.13 [M+H]⁺.

3-[5,7-difluoro-2-(4-fluorophenyl)-1H-indol-3-yl]-N-[(1S)-2,2,2-trifluoro-1-(hydroxymethyl)ethyl]butanamide 514 (5 mg, 20%)¹H NMR (300 MHz, Methanol-d₄) δ 8.32 (d, J=9.3 Hz, 1H), 7.72-7.54 (m, 2H), 7.31 (dd, J=9.9, 2.2 Hz, 1H), 7.28-7.13 (m, 2H), 6.72 (ddd, J=11.1, 9.6, 2.1 Hz, 1H), 4.53 (dd, J=13.4, 5.1 Hz, 1H), 3.74 (dd, J=11.7, 4.8 Hz, 1H), 3.62 (dd, J=12.0, 6.8 Hz, 2H), 2.95-2.64 (m, 2H), 1.42 (d, J=7.1 Hz, 3H). LCMS m/z 445.13 [M+H]⁺.

### Compound 515

### N-[3-[5,7-difluoro-2-(4-fluorophenyl)-1H-indol-3-yl]-2,2-difluoro-propyl]acetamide (515)

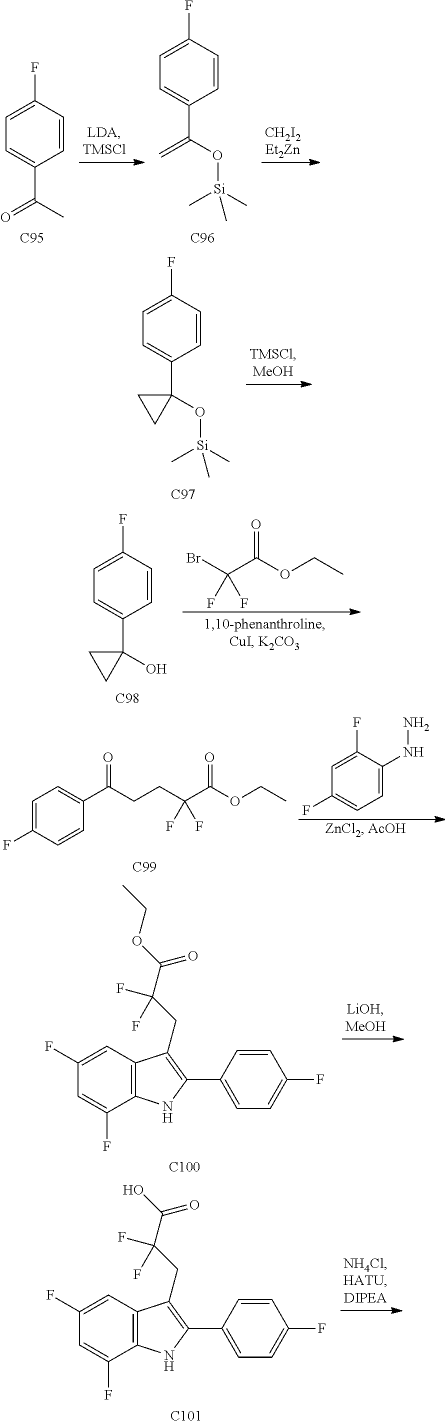

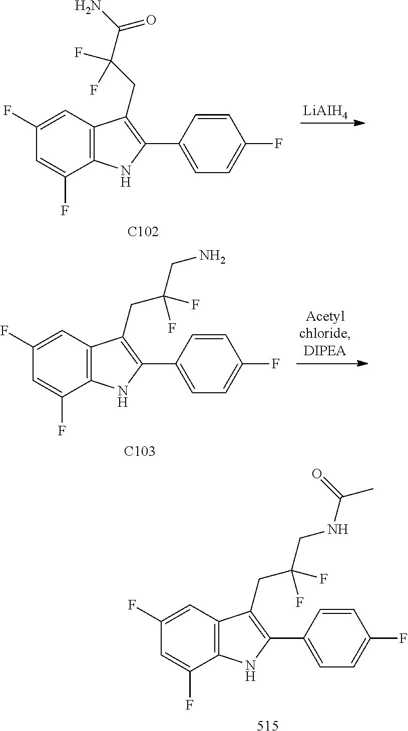

### Step 1. Synthesis of 1-(4-fluorophenyl)vinyloxy-trimethyl-silane (C96)

To a 0° C. solution of (diisopropylamino)lithium (11 mL of 1 M, 11.00 mmol) in THF (9 mL) was added 1-(4-fluorophenyl)ethenone C95 (1.381 g, 10 mmol) followed by TMSCl (1.4 mL, 11.03 mmol). The resulting mixture was warmed to room temperature and stirred for 2 hours then quenched with saturated sodium bicarbonate solution. Extracted with ethyl acetate (3×), washed with sodium bicarbonate, and sodium chloride solutions 3×, then dried over sodium sulfate, filtered, and concentrated in vacuo to afford 1-(4-fluorophenyl)vinyloxy-trimethyl-silane (2.1 g, 100%) LCMS m/z 211.16 [M+H]⁺ which was used without further purification in the next reaction.

### Step 2. Synthesis of [1-(4-fluorophenyl)cyclopropoxy]-trimethyl-silane (C97)

A solution of the 1-(4-fluorophenyl)vinyloxy-trimethyl-silane C96, diiodomethane (1.2 mL, 14.90 mmol), and dichloromethane (20 mL) was degassed with nitrogen and cooled to 0° C. To this solution was added diethylzinc (12.4 mL of 15% w/v, 15.06 mmol). The resulting mixture was warmed to room temperature then stirred overnight. The mixture was quenched with saturated ammonium chloride solution and then extracted DCM (3×). The combined organics were washed with sodium chloride solution, dried over anhydrous sodium sulfate, filtered, and concentrated in vacuo to afford [1-(4-fluorophenyl)cyclopropoxy]-trimethyl-silane (2.2 g, 98%) ¹H NMR (300 MHz, Chloroform-d) δ 7.26-7.13 (m, 2H), 6.98-6.87 (m, 2H), 1.17-1.09 (m, 2H), 0.93-0.87 (m, 2H), 0.08-0.14 (m, 9H). LCMS m/z 224.89 [M+H]⁺ which was used without further purification in the next reaction.

### Step 3. Synthesis of 1-(4-fluorophenyl)cyclopropanol (C98)

The crude [1-(4-fluorophenyl)cyclopropoxy]-trimethyl-silane C97 was dissolved in methanol (20 mL), degassed with nitrogen, and cooled to 0° C. A single drop of chlorotrimethyl silane was added to the reaction mixture, which was then warmed to room temperature and stirred overnight. The mixture was quenched with saturated ammonium chloride and extracted with ethyl acetate (3×). The combined organics were washed with brine, dried over sodium sulfate, filtered, and concentrated in vacuo. The residue was purified via silica gel chromatography (Gradient: 0-30% EtOAc in heptanes) to afford 1-(4-fluorophenyl)cyclopropanol (660 mg, 43%)¹H NMR (300 MHz, Chloroform-d) δ 7.35-7.25 (m, 2H), 7.08-6.98 (m, 2H), 2.34 (s, 1H), 1.31-1.24 (m, 2H), 1.06-0.97 (m, 2H).

### Step 4. Synthesis of ethyl 2,2-difluoro-5-(4-fluorophenyl)-5-oxo-pentanoate (C99)

Under argon atmosphere, 1-(4-fluorophenyl)cyclopropanol C98 (660 mg, 4.337 mmol), ethyl 2-bromo-2,2-difluoro-acetate (3.5 g, 17.24 mmol), iodocopper (84 mg, 0.4411 mmol), 1,10-phenanthroline (158 mg, 0.8768 mmol), and K₂CO₃ (1.2 g, 8.683 mmol) were dissolved in acetonitrile (45 mL) and stirred at 80° C. for 5 hours. The reaction was quenched with water, then partitioned with ethyl acetate. The aqueous layer was separated and extracted with ethyl acetate (3×). The combined organics were washed with saturated NaCl (3×), dried over anhydrous sodium sulfate, filtered, and concentrated in vacuo. The residue was purified via silica gel chromatography (Gradient: 0-20% EtOAc in heptanes) to afford ethyl 2,2-difluoro-5-(4-fluorophenyl)-5-oxo-pentanoate (389 mg, 29%)¹H NMR (300 MHz, Chloroform-d) δ 8.05-7.95 (m, 2H), 7.21-7.07 (m, 2H), 4.33 (q, J=7.1 Hz, 2H), 3.26-3.16 (m, 2H), 2.65-2.46 (m, 2H), 1.35 (t, J=7.2 Hz, 3H). LCMS m/z 275.17 [M+H]⁺.

### Step 5. Synthesis of ethyl 3-[5,7-difluoro-2-(4-fluorophenyl)-1H-indol-3-yl]-2,2-difluoro-propanoate (C100)

A solution of ethyl 2,2-difluoro-5-(4-fluorophenyl)-5-oxo-pentanoate C99 (389 mg, 1.418 mmol), (2,4-difluorophenyl)hydrazine (Hydrochloride salt) (510 mg, 2.824 mmol), and zinc chloride (915 mg, 6.712 mmol) in acetic acid (4 mL) and toluene (4 mL) was heated to 115° C. and stirred overnight. The reaction was concentrated in vacuo then partitioned between ethyl acetate and water. The aqueous layer washed with ethyl acetate (3×) and the combined organics were washed with brine, dried over anhydrous sodium sulfate, filtered, and concentrated in vacuo. The residue was purified via silica gel chromatography (Gradient: 0-20% EtOAc in heptanes) to afford ethyl 3-[5,7-difluoro-2-(4-fluorophenyl)-1H-indol-3-yl]-2,2-difluoro-propanoate (228.4 mg, 40%)¹H NMR (300 MHz, Chloroform-d) δ 8.27 (s, 1H), 7.56 (ddd, J=7.1, 5.3, 2.7 Hz, 2H), 7.25-7.09 (m, 3H), 6.77 (ddd, J=10.7, 9.4, 2.1 Hz, 1H), 4.19 (q, J=7.1 Hz, 2H), 3.53 (t, J=16.9 Hz, 2H), 1.24 (t, J=7.1 Hz, 3H). LCMS m/z 384.15 [M+H]⁺.

### Step 6. Synthesis of 3-[5,7-difluoro-2-(4-fluorophenyl)-1H-indol-3-yl]-2,2-difluoro-propanoic acid (C101)

A solution of ethyl 3-[5,7-difluoro-2-(4-fluorophenyl)-1H-indol-3-yl]-2,2-difluoro-propanoate C100 (210 mg, 0.5205 mmol) in THF (2 mL), MeOH (2 mL), and Water (1 mL) was treated with LiOH (28 mg, 1.169 mmol) and allowed to stir at ambient temperature for 2 hours. The reaction was concentrated in vacuo, then the aqueous layer was acidified with 1 M HCl to pH 3, followed by extraction with ethyl acetate (3×). The combined organics were washed with brine, dried over anhydrous sodium sulfate, filtered, and concentrated in vacuo to afford 3-[5,7-difluoro-2-(4-fluorophenyl)-1H-indol-3-yl]-2,2-difluoro-propanoic acid (155.3 mg, 80%)¹H NMR (300 MHz, Chloroform-d) δ 8.28 (s, 1H), 7.54 (ddd, J=8.8, 5.0, 2.3 Hz, 2H), 7.27-7.10 (m, 6H), 6.78 (ddd, J=10.7, 9.4, 2.2 Hz, 1H), 3.53 (d, J=17.1 Hz, 2H). LCMS m/z 356.02 [M+H]⁺.

### Step 7. Synthesis of 3-[5,7-difluoro-2-(4-fluorophenyl)-1H-indol-3-yl]-2,2-difluoro-propanamide (C102)

A solution of 3-[5,7-difluoro-2-(4-fluorophenyl)-1H-indol-3-yl]-2,2-difluoro-propanoic acid C101 (102.1 mg, 0.2730 mmol), ammonium chloride (45 mg, 0.8413 mmol), HATU (157 mg, 0.4129 mmol), and DMF (2 mL) was treated with DIPEA (143 μL, 0.8210 mmol). The resulting solution was stirred at room temperature for 1.5 hours, then partitioned between ethyl acetate and water. The organics were concentrated in vacuo, combined with the crude from a smaller scale version of this reaction (50 mg of 3-[5,7-difluoro-2-(4-fluorophenyl)-1H-indol-3-yl]-2,2-difluoro-propanoic acid and purified via silica gel chromatography (Gradient: 0-100% EtOAc in heptanes) to afford ethyl 3-[5,7-difluoro-2-(4-fluorophenyl)-1H-indol-3-yl]-2,2-difluoro-propanamide (136.9 mg, 95%)¹H NMR (300 MHz, DMSO-d₆) δ 11.97 (s, 1H), 8.01 (d, J=64.0 Hz, 2H), 7.76-7.63 (m, 2H), 7.45-7.31 (m, 2H), 7.22 (ddd, J=9.4, 7.6, 2.2 Hz, 1H), 7.01 (ddd, J=11.2, 9.7, 2.2 Hz, 1H), 3.52 (t, J=17.9 Hz, 2H). LCMS m/z 355.07 [M+H]⁺.

### Step 8. Synthesis of 3-[5,7-difluoro-2-(4-fluorophenyl)-1H-indol-3-yl]-2,2-difluoro-propan-1-amine (C103)

A solution of 3-[5,7-difluoro-2-(4-fluorophenyl)-1H-indol-3-yl]-2,2-difluoro-propanamide C102 (103.9 mg, 0.2786 mmol) in THF (2 mL) under N₂ was cooled to 0° C. in ice bath. Lithium aluminum hydride (560 μL of 2 M, 1.120 mmol) was added to the reaction. The ice bath was removed, and the reaction slowly warmed to room temperature then heated at reflux for 2.5 hours. The reaction was cooled to room temperature then 0° C. in an ice bath. The reaction was slowly quenched by addition of saturated Rochelle's salt followed by ethyl acetate. The organics were separated and the aqueous washed with ethyl acetate (3×), the combined organics were washed with brine, dried over anhydrous sodium sulfate, filtered, and concentrated in vacuo to afford 3-[5,7-difluoro-2-(4-fluorophenyl)-1H-indol-3-yl]-2,2-difluoro-propan-1-amine (70.9 mg, 69%) LCMS m/z 341.12 [M+H]⁺.

### Step 9. Synthesis of N-[3-[5,7-difluoro-2-(4-fluorophenyl)-1H-indol-3-yl]-2,2-difluoro-propyl]-acetamide (515)

A solution of 3-[5,7-difluoro-2-(4-fluorophenyl)-1H-indol-3-yl]-2,2-difluoro-propan-1-amine C₁₀₃ (70.9 mg, 0.1926 mmol) in dichloromethane (2 mL) was treated with acetyl chloride (15 μL, 0.2110 mmol) followed by DIPEA (51 μL, 0.2928 mmol). The resulting solution was stirred at room temperature overnight then partitioned between dichloromethane and 1N NaOH. The organic layer was concentrated in vacuo then purified via silica gel chromatography (Gradient: 0-20% EtOAc in heptanes) to afford ethyl N-[3-[5,7-difluoro-2-(4-fluorophenyl)-1H-indol-3-yl]-2,2-difluoro-propyl]acetamide (22.5 mg, 30%). ¹H NMR (300 MHz, DMSO-d₆) δ 11.94 (s, 1H), 8.27 (t, J=6.1 Hz, 1H), 7.70-7.65 (m, 2H), 7.40-7.34 (m, 2H), 7.24-7.19 (m, 1H), 7.01 (ddd, J=11.2, 9.8, 2.3 Hz, 1H), 3.57 (td, J=14.7, 6.1 Hz, 2H), 3.36 (s, 2H), 1.85 (s, 3H). LCMS m/z 383.11 [M+H]⁺.

### Compound 516

### N-(2,3-dihydroxypropyl)-2-(2-(4-fluorophenyl)-1H-indol-3-yl)acetamide (516)

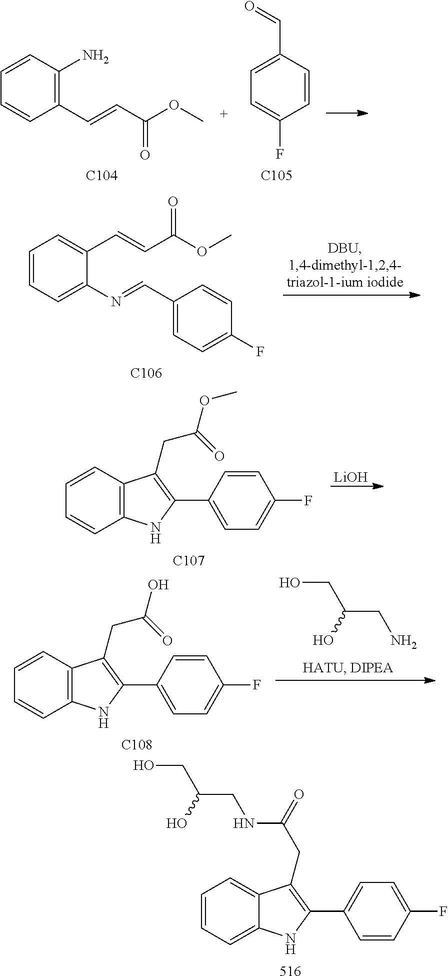

### Step 1. Synthesis of methyl (E)-3-[2-[(E)-(4-fluorophenyl)methyleneamino]phenyl]prop-2-enoate (C106)

Anhydrous 4 Å molecular sieves were flame dried in flask under high vacuum. The flask was cooled under high vacuum then charged with methyl (E)-3-(2-aminophenyl)prop-2-enoate C104 (1 g, 5.643 mmol) and 4-fluorobenzaldehyde C105 (596 μL, 5.647 mmol), followed by anhydrous toluene (30 mL). The resulting solution was stirred at reflux overnight under nitrogen. The solution was concentrated in vacuo then dried overnight under high vacuum to afford product as a yellow oil methyl (E)-3-[2-[(E)-(4-fluorophenyl)methyleneamino]-phenyl]prop-2-enoate (1.01 g, 60%)¹H NMR (300 MHz, Chloroform-d) δ 8.35 (s, 1H), 8.19 (d, J=16.1 Hz, 1H), 7.99-7.91 (m, 2H), 7.63 (dd, J=7.8, 1.5 Hz, 1H), 7.41 (td, J=7.7, 1.5 Hz, 1H), 7.26 (dd, J=7.6, 1.3 Hz, 1H), 7.23-7.15 (m, 2H), 7.00 (dd, J=7.9, 1.3 Hz, 1H), 6.47 (d, J=16.2 Hz, 1H), 3.79 (s, 3H). LCMS m/z 284.8 [M+H]⁺.

### Step 2. Synthesis of methyl 2-[2-(4-fluorophenyl)-1H-indol-3-yl]acetate (C107)

A solution of methyl (E)-3-[2-[(E)-(4-fluorophenyl)methyleneamino]phenyl]prop-2-enoate C106 (1.01 g, 3.387 mmol) in anhydrous THF (27 mL) was treated with DBU (608 μL, 4.066 mmol) and 1,4-dimethyl-1,2,4-triazol-1-ium iodide (230 mg, 1.022 mmol) under nitrogen. The resulting solution was heated/stirred at 80° C. for 4 hours. The reaction was quenched by partitioning between ethyl acetate and water, the combined organics were dried over anhydrous sodium sulfate, filtered, and concentrated in vacuo. The residue was purified via silica gel chromatography (Gradient: 10-100% EtOAc in heptanes) to afford methyl 2-[2-(4-fluorophenyl)-1H-indol-3-yl]acetate (716.1 mg, 70%)¹H NMR (300 MHz, Chloroform-d) δ 8.13 (s, 1H), 7.71-7.63 (m, 3H), 7.40 (dt, J=8.0, 1.0 Hz, 1H), 7.27-7.17 (m, 4H), 3.83 (s, 2H), 3.74 (s, 3H). LCMS m/z 284.13 [M+H]⁺.

### Step 3. Synthesis of 2-[2-(4-fluorophenyl)-1H-indol-3-yl]acetic acid (C108)

A solution of methyl 2-[2-(4-fluorophenyl)-1H-indol-3-yl]acetate C₁₀₇ (4.5 g, 14.69 mmol) in THF (50 mL) and MeOH (50 mL) and Water (25 mL) was treated with LiOH (780 mg, 32.57 mmol). The resulting mixture was stirred at ambient temperature overnight. The reaction was quenched by concentration in vacuo of organics, then acidification of the aqueous layer with 1N HCl to pH 3. The resulting thick white precipitate was collected under vacuum filtration, washed with water, filtered, and dried to constant mass to afford 2-[2-(4-fluorophenyl)-1H-indol-3-yl]acetic acid (3.8902 g, 94%)¹H NMR (300 MHz, DMSO-d₆) δ 12.38 (s, 1H), 11.34 (s, 1H), 7.79-7.67 (m, 2H), 7.55 (d, J=7.8 Hz, 1H), 7.45-7.33 (m, 3H), 7.08 (dddd, J=29.7, 8.0, 7.0, 1.2 Hz, 2H). LCMS m/z 270.35 [M+H]⁺.

### Step 4. Synthesis of N-(2,3-dihydroxypropyl)-2-(2-(4-fluorophenyl)-1H-indol-3-yl)acetamide (516)

A solution of the 2-[2-(4-fluorophenyl)-1H-indol-3-yl]acetic acid C108 (25 mg) and HATU (42 mg) and DIPEA (33 μL) in DMF (1.0 mL) was added to a tube containing 3-aminopropane-1,2-diol. The reaction was agitated on an orbital shaker overnight. The reaction was then directly submitted to purification by reversed-phase HPLC (Method: C18 Waters Sunfire column (30×150 mm, 5 micron). Gradient: MeCN in H₂O with 0.1% trifluoroacetic acid) to afford N-(2,3-dihydroxypropyl)-2-(2-(4-fluorophenyl)-1H-indol-3-yl)acetamide. LCMS m/z 343.77 [M+H]⁺.

### Compound 517

### 3-[5,7-difluoro-2-(4-fluorophenyl)-1H-indol-3-yl]propan-1-ol (517)

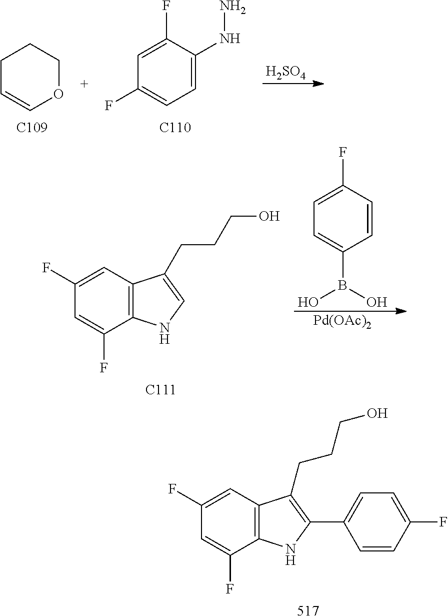

### Step 1. Synthesis of 3-(5,7-difluoro-1H-indol-3-yl)propan-1-ol (C111)

In a 250 mL round bottle flask, 3,4-dihydro-2H-pyran (3.8 mL, 41.65 mmol) and (2,4-difluorophenyl)hydrazine C110 (5 g, 34.69 mmol) were dissolved in DMA (100 mL) and H₂SO₄ (100 mL, 1.876 mol) was added. The mixture was heated to 100° C. for 2 hours. The reaction mixture was then cooled to room temperature, and water (250 mL) was added to the reaction mixture, followed by extraction with EtOAc (3×250 mL). Combined organic fractions were dried over sodium sulfate, after filtered off salt, the solution was concentrated in vacuo. Purification by silica gel chromatography (Gradient: 10-100% EtOAc in hexanes) to afford 3-(5,7-difluoro-1H-indol-3-yl)propan-1-ol (4.2 g, 47%). LCMS m/z 212.02 [M+H]⁺.

### Step 2. Synthesis of 3-[5,7-difluoro-2-(4-fluorophenyl)-1H-indol-3-yl]propan-1-ol (517)

In a 20 mL microwave tube, (4-fluorophenyl)boronic acid (78 mg, 0.56 mmol) and 3-(5,7-difluoro-1H-indol-3-yl)propan-1-ol C111 (115 mg, 0.545 mmol) were dissolved in AcOH (3 mL). Then palladium acetate (15 mg, 0.06681 mmol) was added to the reaction mixture. The reaction mixture was degassed with 02 for 5 minutes, then the tube was sealed, and the mixture was stirred at room temperature for overnight. The solvent was then removed reduced pressure. Saturated NaHCO₃ solution was added to the reaction mixture, followed by extraction with DCM (3×30 mL). Combined organic fractions were washed with NaHCO₃ (20 mL), dried over MgSO4. Purification by reversed-phase HPLC (Method: C18 column. Gradient: MeCN in H₂O with 0.1% trifluoroacetic acid) provided 3-[5,7-difluoro-2-(4-fluorophenyl)-1H-indol-3-yl]propan-1-ol (2.5 mg, 3%). ¹H NMR (300 MHz, Chloroform-d) δ 8.11 (s, 1H), 7.63-7.49 (m, 2H), 7.21 (t, J=8.6 Hz, 2H), 7.11 (dd, J=9.1, 2.2 Hz, 1H), 6.77 (ddd, J=10.8, 9.4, 2.2 Hz, 1H), 3.69 (t, J=6.3 Hz, 2H), 3.00-2.83 (m, 2H), 2.04-1.84 (m, 2H), 1.28 (s, 1H). LCMS m/z 306.19 [M+H]⁺.

### Compound 518

### (E)-3-[5,7-difluoro-2-(4-fluorophenyl)-1H-indol-3-yl]-N-[(3S,4R)-4-hydroxy-2-oxo-pyrrolidin-3-yl]prop-2-enamide (518)

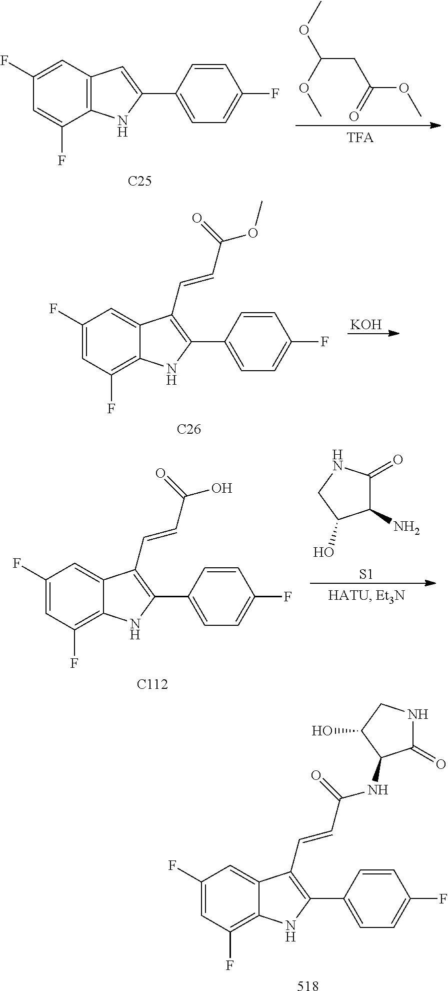

### Step 1. Synthesis of methyl (E)-3-[5,7-difluoro-2-(4-fluorophenyl)-1H-indol-3-yl]prop-2-enoate (C26)

A mixture of 5,7-difluoro-2-(4-fluorophenyl)-1H-indole (1.38 g, 5.582 mmol), DCM (15 mL), methyl 3,3-dimethoxypropanoate (900 μL, 6.348 mmol) and TFA (2.4 mL, 31.15 mmol) was heated to reflux for 14 hours. The reaction mixture was then cooled to room temperature and filtered, to collect the product as the solid: methyl (E)-3-[5,7-difluoro-2-(4-fluorophenyl)-1H-indol-3-yl]prop-2-enoate (1.6 g, 86%). LCMS m/z 332.21 [M+H]⁺.

### Step 2. Synthesis of (E)-3-[5,7-difluoro-2-(4-fluorophenyl)-1H-indol-3-yl]prop-2-enoic acid (C112)

A mixture of methyl (E)-3-[5,7-difluoro-2-(4-fluorophenyl)-1H-indol-3-yl]prop-2-enoate C26 (840 mg, 2.527 mmol) and KOH (1.6 g, 28.52 mmol) in MeOH (15 mL) and H₂O (18 mL) was heated at 100° C. for 6 hours. The reaction mixture was then concentrated in vacuo. Concentrated HCl was added to adjust till pH=1. The mixture was then filtered, washed with water (3×), dried to yield product (E)-3-[5,7-difluoro-2-(4-fluorophenyl)-1H-indol-3-yl]prop-2-enoic acid (765 mg, 94%). LCMS m/z 318.12 [M+H]⁺.

### Step 3. Synthesis of (E)-3-[5,7-difluoro-2-(4-fluorophenyl)-1H-indol-3-yl]-N-[(3S,4R)-4-hydroxy-2-oxo-pyrrolidin-3-yl]prop-2-enamide (518)

To (E)-3-[5,7-difluoro-2-(4-fluorophenyl)-1H-indol-3-yl]prop-2-enoic acid C112 (65 mg, 0.2033 mmol), (3S,4R)-3-amino-4-hydroxy-pyrrolidin-2-one S1 (24 mg, 0.2067 mmol) and HATU (93 mg, 0.2446 mmol) in DMSO (1 mL) was added Et₃N (115 μL, 0.8251 mmol). The reaction mixture was stirred at room temperature for 6 h. An additional HATU (46 mg, 0.12 mmol) and Et₃N (58 μL, 0.42 mmol) were added. After another 6 h, the reaction mixture was directly purified by reversed-phase HPLC (Method: C18 Waters Sunfire column (30×150 mm, 5 micron). Gradient: MeCN in H₂O with 0.1% trifluoroacetic acid) to give the product. (E)-3-[5,7-difluoro-2-(4-fluorophenyl)-1H-indol-3-yl]-N-[(3S,4R)-4-hydroxy-2-oxo-pyrrolidin-3-yl]prop-2-enamide (12.2 mg, 14%). ¹H NMR (300 MHz, Methanol-d₄) δ 12.01 (s, 1H), 7.79 (dd, J=15.8, 0.6 Hz, 1H), 7.71-7.56 (m, 2H), 7.52 (dd, J=9.8, 2.2 Hz, 1H), 7.39-7.20 (m, 2H), 6.73 (d, J=15.8 Hz, 1H), 4.49 (td, J=7.6, 6.9 Hz, 1H), 4.37 (d, J=7.8 Hz, 1H), 3.64 (dd, J=9.9, 7.6 Hz, 1H), 3.17 (dd, J=9.9, 7.0 Hz, 1H). LCMS m/z 416.0 [M+H]⁺.

### Compound 519

### (E)-3-[5,7-difluoro-2-(4-fluorophenyl)-1H-indol-3-yl]-N-[(3S)-2-oxopyrrolidin-3-yl]prop-2-enamide (519)

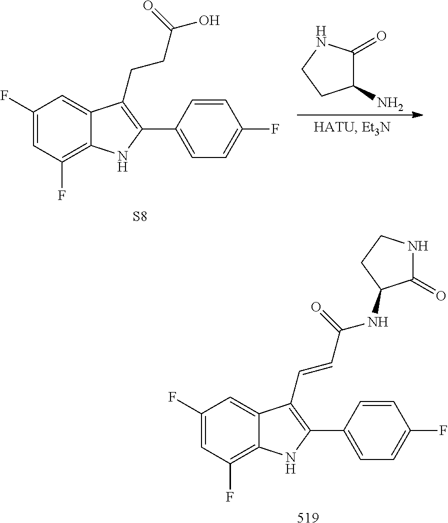 [SMILES: O=C(O)CCc1c(-c2ccc(F)cc2)[nH]c2c(F)cc(F)cc12 ; N[C@H]1CCNC1=O ; "S8" ; O=C(/C=C/c1c(-c2ccc(F)cc2)[nH]c2c(F)cc(F)cc12)N[C@H]1CCNC1=O ; "519"]

### Preparation of (E)-3-[5,7-difluoro-2-(4-fluorophenyl)-1H-indol-3-yl]-N-[(3S)-2-oxopyrrolidin-3-yl]prop-2-enamide (519)

To 3-[5,7-difluoro-2-(4-fluorophenyl)-1H-indol-3-yl]propanoic acid S8 (1280 mg, 3.809 mmol), (3S)-3-aminopyrrolidin-2-one (470 mg, 4.694 mmol), HATU (2.1 g, 5.523 mmol) in DMF (20 mL) was added Et₃N (2 mL, 14.35 mmol). The reaction was stirred at room temperature for 5 hours. The reaction mixture was concentrated in vacuo. Then it was added EtOAc (300 mL), wash with 0.5 M HCl (100 mL), brine, and dried over Na₂SO₄. The organic layer was concentrated in vacuo. Purification by silica gel chromatography (Gradient: 0-10% MeOH in EtOAc). Second purification was performed using silica gel chromatography (Gradient: 0-20% MeOH in DCM). The title compound was isolated as a minor product: (E)-3-[5,7-difluoro-2-(4-fluorophenyl)-1H-indol-3-yl]-N-[(3S)-2-oxopyrrolidin-3-yl]prop-2-enamide (16 mg, 1%). ¹H NMR (300 MHz, Chloroform-d) δ 8.79 (s, 1H), 7.81 (d, J=15.6 Hz, 1H), 7.62-7.51 (m, 2H), 7.34 (dd, J=9.4, 2.1 Hz, 1H), 7.22 (t, J=8.6 Hz, 2H), 6.86 (ddd, J=11.1, 9.2, 2.1 Hz, 1H), 6.41 (d, J=15.6 Hz, 1H), 6.27 (d, J=5.4 Hz, 1H), 5.78 (s, 1H), 4.50 (ddd, J=11.0, 8.2, 5.3 Hz, 1H), 3.46 (dd, J=9.8, 4.3 Hz, 2H), 2.89 (dd, J=7.9, 4.7 Hz, 1H), 2.17-1.94 (m, 1H). LCMS m/z 400.17 [M+H]⁺.

### Compound 520

### (E)-3-[4,7-difluoro-2-(4-fluorophenyl)-1H-indol-3-yl]-N-[(3S)-2-oxopyrrolidin-3-yl]prop-2-enamide (520)

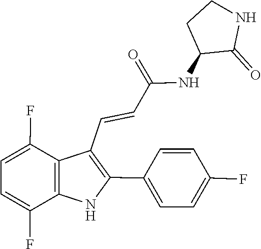 [SMILES: O=C(/C=C/c1c(-c2ccc(F)cc2)[nH]c2c(F)ccc(F)c12)N[C@H]1CCNC1=O]

Compound 520 was prepared in three steps from C47 and (3S)-3-aminopyrrolidin-2-one using the method described for the preparation of compound 519. LCMS m/z 400.11 [M+H]⁺.

### Compound 521

### Methyl (E)-3-[2-(4-cyanophenyl)-5,7-difluoro-1H-indol-3-yl]prop-2-enoate (521)

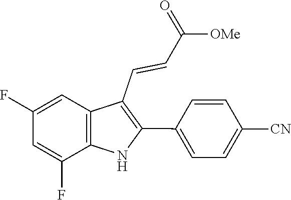 [SMILES: COC(=O)/C=C/c1c(-c2ccc(C#N)cc2)[nH]c2c(F)cc(F)cc12]

Compound 521 was isolated as a minor product in the preparation of compound C51 described above. Methyl (E)-3-[2-(4-cyanophenyl)-5,7-difluoro-1H-indol-3-yl]prop-2-enoate (153 mg, 18%). ¹H NMR (300 MHz, Chloroform-d) δ 8.67 (s, 1H), 7.93-7.80 (m, 3H), 7.78-7.66 (m, 2H), 7.45 (dd, J=9.3, 2.2 Hz, 1H), 6.92 (ddd, J=10.5, 9.2, 2.1 Hz, 1H), 6.52 (d, J=16.1 Hz, 1H), 3.83 (s, 3H). LCMS m/z 339.09 [M+H]⁺

### Compound 522

### (E)-3-[2-(4-cyanophenyl)-5,7-difluoro-1H-indol-3-yl]-N-[(3S)-2-oxopyrrolidin-3-yl]prop-2-enamide (522)

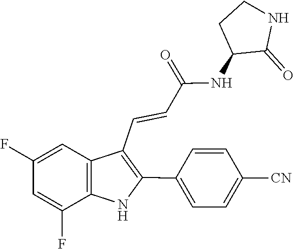 [SMILES: N#Cc1ccc(-c2[nH]c3c(F)cc(F)cc3c2/C=C/C(=O)N[C@H]2CCNC2=O)cc1]

To a mixture of 3-[2-(4-cyanophenyl)-5,7-difluoro-1H-indol-3-yl]propanoic acid S13 (45 mg, 0.1355 mmol) HATU (103 mg, 0.2709 mmol) and (3S)-3-aminopyrrolidin-2-one (27 mg, 0.2697 mmol) DMSO (2 mL) was added TEA (95 μL, 0.6816 mmol). The reaction was allowed to stir at room temperature for room temperature for 12 hours. Purification by reverse phase chromatography afforded the product as a minor component. E)-3-[2-(4-cyanophenyl)-5,7-difluoro-1H-indol-3-yl]-N-[(3S)-2-oxopyrrolidin-3-yl]prop-2-enamide (10 mg, 18%). ¹H NMR (300 MHz, Methanol-d₄) δ 7.58-7.50 (m, 2H), 7.44-7.38 (m, 2H), 7.27-7.20 (m, 1H), 7.13 (d, J=10.1 Hz, 1H), 6.78-6.64 (m, 1H), 5.50-5.36 (m, 1H), 4.33 (t, J=7.8 Hz, 1H), 3.25-3.17 (m, 1H), 2.25-2.09 (m, 1H), 1.65-1.52 (m, 1H). LCMS m/z 407.14 [M+H]⁺.

### Compound 523

### 4-[5,7-difluoro-2-(4-fluorophenyl)-1H-indol-3-yl]-2-methyl-but-3-yn-2-ol (523)

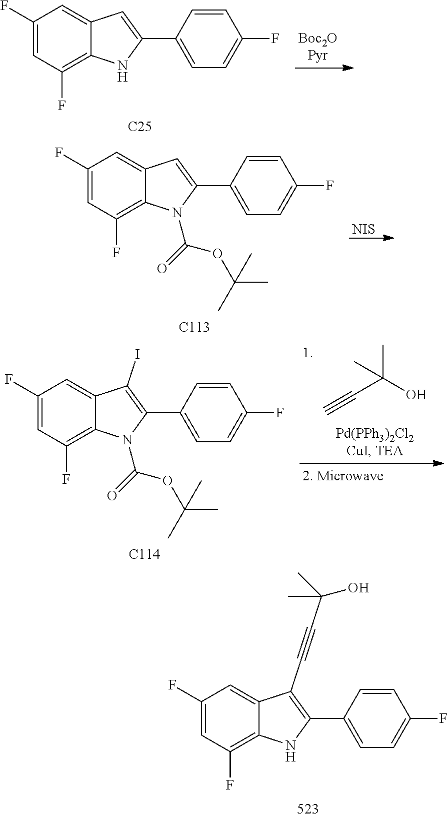

### Step 1: Synthesis of tert-butyl 5,7-difluoro-2-(4-fluorophenyl)indole-1-carboxylate (C113)

To a solution of ditert-butyl carbonate (5 g, 28.70 mmol) and 5,7-difluoro-2-(4-fluorophenyl)-1H-indole (6 g, 24.27 mmol) in THF (120 mL) was added Pyridine (4 mL, 49.46 mmol), followed by DMAP (295 mg, 2.415 mmol). The reaction mixture was stirred at room temperature overnight. The solvent was removed solvent under reduced pressure. Water was added to the reaction mixture followed by extraction with EtOAc (3×50 mL). Combined organic fractions were washed with H₂O (1×20 mL), brine (1×20 mL), dried over sodium sulfate, after filtered off salt, the solution was concentrated to dryness. Purification Silica (Redi-Sep cartridge, 120 g), eluting with 0-60% EtOAc in Hexanes to afford tert-butyl 5,7-difluoro-2-(4-fluorophenyl)indole-1-carboxylate (8.3 g, 96%). LCMS m/z 345.64 [M+1]⁺.

### Step 2: Synthesis of tert-butyl 5,7-difluoro-2-(4-fluorophenyl)-3-iodo-indole-1-carboxylate (C114)

was dissolved in CHCl₃ (50 mL), the mixture was cooled to 0° C., to which, 1-iodopyrrolidine-2,5-dione (2.3 g, 10.22 mmol) was added. The mixture was stirred at 0° C. for 3 hours, the stirred at room temperature overnight. Additional 1-iodopyrrolidine-2,5-dione was added to the reaction mixture, which was allowed to stir until completion. The reaction mixture was quenched with sat. Na₂SO₃ (20 mL). Diluted with 30 mL of H₂O, the organic layer was separated, the aqueous layer was extracted with DCM (3×50 mL), dried over Na₂SO₄, Filtered off Na₂SO₄ and removed solvent to afford the off white solid, which was washed with Heptane to afford clean compound. tert-butyl 5,7-difluoro-2-(4-fluorophenyl)-3-iodo-indole-1-carboxylate (4.1 g, 91%). ¹H NMR (300 MHz, Chloroform-d) δ 7.51-7.36 (m, 2H), 7.26-7.15 (m, 2H), 7.02 (ddd, J=8.0, 2.4, 0.8 Hz, 1H), 6.93 (ddt, J=12.0, 9.3, 2.6 Hz, 1H), 1.37 (d, J=2.5 Hz, 9H). LCMS m/z 472.86 [M+H]⁺.

### Step 3. Synthesis of 5,7-fluoro-2-(4-fluorophenyl)-3-[3-(1-hydroxycyclobutyl)prop-1-ynyl]indole-1-carboxylate

1 tert-Butyl 5,7-difluoro-2-(4-fluorophenyl)-3-iodo-indole-1-carboxylate (133 mg, 0.2810 mmol) and 1-prop-2-ynylcyclobutanol (115 mg, 1.044 mmol) were dissolved in N-ethylethanamine (6 mL), the mixture was degassed with Na for several minutes, then Pd(PPh₃)₂Cl₂ (20 mg, 0.02849 mmol) and CuI (11 mg, 0.05776 mmol) were added. Sealed the tube and heated the reaction mixture to 60° C. for overnight. The mixture was concentrated. Water (10 mL) was added to the reaction mixture followed by extraction with EtOAc (3×10 mL). Combined organic fractions were washed with H₂O (1×2 mL), brine (1×2 mL), dried over sodium sulfate, filtered and concentrated to dryness. Purification by silica gel (Gradient: 0-10% EtOAc in Hexanes) afforded tert-butyl 5,7-difluoro-2-(4-fluorophenyl)-3-[3-(1-hydroxycyclobutyl)prop-1-ynyl]indole-1-carboxylate (121 mg, 83%). ¹H NMR (300 MHz, Chloroform-d) δ 7.61-7.42 (m, 2H), 7.25-7.08 (m, 3H), 6.88 (ddd, J=11.7, 9.3, 2.4 Hz, 1H), 2.72 (s, 2H), 2.14-2.02 (m, 4H), 1.89-1.68 (m, 2H), 1.65-1.47 (m, 1H), 1.41 (s, 9H).

### Step 4: Synthesis of 1-[3-[5,7-difluoro-2-(4-fluorophenyl)-1H-indol-3-yl]prop-2-ynyl]cyclobutanol (523)

tert-butyl 5,7-difluoro-2-(4-fluorophenyl)-3-[3-(1-hydroxycyclobutyl)prop-1-ynyl]in-dole-1-carboxylate (60 mg, 0.1161 mmol) was dissolved in DCM (2 mL) to which, TFA (500 μL, 6.490 mmol) was added. The mixture was stirred at RT for 1 hour, Removed the solvent, Purification by silica gel chromatography (Gradient: 0-100% EtOAc in Hexanes) afforded 4-[5,7-difluoro-2-(4-fluorophenyl)-1H-indol-3-yl]-2-methyl-but-3-yn-2-ol (5.6 mg, 5%) as the minor product. ¹H NMR (300 MHz, Chloroform-d) δ 8.48 (s, 1H), 8.06-7.89 (m, 2H), 7.26-7.13 (m, 3H), 6.80 (ddd, J=10.7, 9.3, 2.2 Hz, 1H), 2.12 (s, 1H), 1.70 (s, 6H). LCMS m/z 330.49 [M+H]⁺. 1-[5,7-difluoro-2-(4-fluorophenyl)-1H-indol-3-yl]-3-(1-hydroxycyclobutyl)propan-1-one (38 mg, 70%) was obtained as the major product. ¹H NMR (300 MHz, Chloroform-d) δ 8.92 (s, 1H), 7.81 (dd, J=9.6, 2.2 Hz, 1H), 7.69-7.44 (m, 2H), 7.35-7.12 (m, 2H), 6.85 (ddd, J=11.1, 9.3, 2.2 Hz, 1H), 2.63 (t, J=6.6 Hz, 2H), 2.11-1.78 (m, 6H), 1.78-1.58 (m, 1H), 1.40 (dp, J=11.3, 8.9 Hz, 1H). LCMS m/z 374.24 [M+H]⁺.

### Compound 524

### 3-[3-[5,7-difluoro-2-(4-fluorophenyl)-1H-indol-3-yl]prop-2-ynyl]oxetan-3-ol (524)

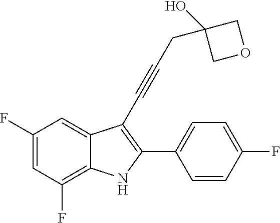 [SMILES: OC1(CC#Cc2c(-c3ccc(F)cc3)[nH]c3c(F)cc(F)cc23)COC1]

Compound 524 was prepared from C114 and the appropriate alkyne using the method described for the preparation of compound 523.

3-[3-[5,7-difluoro-2-(4-fluorophenyl)-1H-indol-3-yl]prop-2-ynyl]oxetan-3-ol (26 mg, 64%). ¹H NMR (300 MHz, Methanol-d₄) δ 8.24-8.05 (m, 2H), 7.27-7.16 (m, 2H), 7.13 (dd, J=8.9, 2.3 Hz, 1H), 6.79 (ddd, J=11.5, 9.7, 2.3 Hz, 1H), 4.74 (d, J=6.6 Hz, 2H), 4.65 (d, J=6.6 Hz, 2H), 2.99 (s, 2H). LCMS m/z 358.02 [M+H]⁺;

### Compound 525

### 1-[3-[5,7-difluoro-2-(4-fluorophenyl)-1H-indol-3-yl]prop-2-ynyl]cyclobutanol (525)

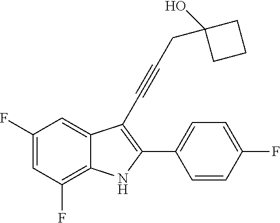 [SMILES: OC1(CC#Cc2c(-c3ccc(F)cc3)[nH]c3c(F)cc(F)cc23)CCC1]

Compound 525 was prepared from C114 and the appropriate alkyne using the method described for the preparation of compound 523.

1-[3-[5,7-difluoro-2-(4-fluorophenyl)-1H-indol-3-yl]prop-2-ynyl]cyclobutanol (9.2 mg, 19%). ¹H NMR (300 MHz, Chloroform-d) δ 8.46 (s, 1H), 8.04-7.87 (m, 2H), 7.18 (dtd, J=8.7, 6.7, 2.2 Hz, 3H), 6.78 (ddd, J=10.7, 9.4, 2.2 Hz, 1H), 2.88 (s, 2H), 2.37 (s, 1H), 2.28-2.17 (m, 4H), 1.97-1.79 (m, 1H), 1.73-1.66 (m, 1H). LCMS m/z 356.24 [M+1]⁺.

### Compound 526

Compound 526 was obtained from commercial sources, and may be prepared from S7 using analogous methods to that described for compound 1.

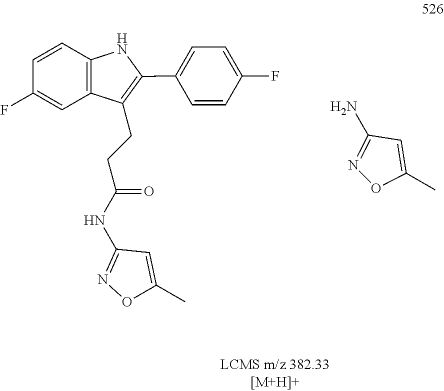

### Compound 527

### N-[2-(2-phenyl-1H-indol-3-yl)ethyl]acetamide (527)

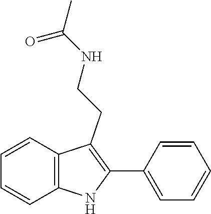 [SMILES: CC(=O)NCCc1c(-c2ccccc2)[nH]c2ccccc12]

Compound 527 was obtained from commercial sources. Compound 525 may be prepared using methods described for compound 431. ¹H NMR (400 MHz, DMSO-d₆) δ 11.19 (s, 1H), 8.05 (t, J=5.9 Hz, 1H), 7.75-7.65 (m, 2H), 7.60 (d, J=7.9 Hz, 1H), 7.49 (d, J=7.9 Hz, 2H), 7.38 (td, J=8.3, 1.8 Hz, 2H), 7.11 (ddd, J=8.1, 7.0, 1.2 Hz, 1H), 7.02 (d, J=7.0 Hz, 1H), 3.33-3.27 (m, 2H), 3.02-2.89 (m, 2H), 1.79 (d, J=2.9 Hz, 3H). LCMS m/z 279.25 [M+H]

### Example 2. Assays for Detecting and Measuring APOL1 Inhibitor Properties of Compounds

Acute APOL1 Thallium Assay with Inducible Stable Clones of HEK 293 Cells

Apolipoprotein L1 (APOL1) proteins form potassium-permeable cation pores in the plasma membrane. APOL1 risk variants (G1 and G2) induce greater potassium flux than G0 in HEK293 cells. This assay exploits the permeability of thallium (Tl+) through ligand-gated potassium channels. The dye produces a bright fluorescent signal upon binding to Tl+ conducted through potassium channels. The intensity of the Tl+ signal is proportional to the number of potassium channels in the open state. Therefore, it provides a functional indication of the potassium channel activities. During the initial dye-loading step, the Tl+ indicator dye as an acetoxymethyl (AM) ester enters the cells through passive diffusion. Cytoplasm esterases cleave the AM ester and relieve its active thallium-sensitive form. The cells are then stimulated with Tl+. The increase of fluorescence in the assay represents the influx of Tl+ into the cell specifically through the potassium channel (i.e. through APOL1 pores), providing a functional measurement of potassium channel/pore activity. The Thallium assay is conducted with cells expressing G1 APOL1.

### Reagents and Materials

APOL1 Cell Line (HEK T-Rex Stable Inducible Cell Line)
HEK T-Rex System
   Tetracycline (Tet) inducible mammalian expression system.
   Stably express the Tet repressor to regulate transcription.
   Expression under the full-length CMV promoter.
APOL1 stable inducible cell line Clone used: G1 DC3.25

Tissue Culture Media
Cell Culture Medium
   DMEM+10% FBS+P/S+5 μg/mL blasticidin+1 μg/mL puromycin.
   500 mL DMEM+55 mL FBS+5 mL P/S+280 μL blasticidin S HCl (10 mg/mL)+56 μL puromycin (10 mg/mL).
Cell Assay Medium
   DMEM with 2% FBS+penicillin/streptomycin.

Reagents:

| | | |
|---|---|---|
| PBS | 7.4 pH | Gibco Cat. No. 10-010- |
| | no phenol red | 49 |
| | no sodium pyruvate | |
| | Concentration: 1X | |
| Trypsin | 0.25%/EDTA 2.21 mM | Wisent, Cat. No. 325- |
| | in HBSS | 043-EL |
| DMEM | High Glucose, no sodium | GIBCO, Cat. No. 11960- |
| | pyruvate, with phenol | 051 |
| | red, with glutamine | |
| FBS | Tet System Approved FBS | Takara Cat. No. 631101 |
| | US Sourced | |
| HEPES Buffer | 1M | Invitrogen, Cat. No. |
| | | 15630-080 |
| HBSS | calcium | Life Technologies, Cat. |
| | magnesium | No. 14025-126 |
| | no phenol red | |
| DMSO | | |
| Penicillin | Sterile filtered for cell | Wisent, Cat. No. 450- |
| Streptomycin | culture | 201-EL |
| (P/S) | Concentration: 100X | |
| Puromycin | Concentration: 10 mg/mL | Gibco, Cat. No. A11138- |
| Dihydrochloride | | 03 |
| Blasticidin S HCl | Concentration: 10 mg/mL | Gibco, Cat. No. A11139- |
| | | 03 |
| Ouabain | Prepare 100 mM stock in | Tocris, Cat. No. 1076 |
| | DMSO | |
| | aliquot and store at −20° C. | |
| Probenecid | Resuspend in 1 mL HBSS | Invitrogen, Cat. No. |
| | 20 mM HEPES | P36400 |
| Tetracycline | Prepare 1 mg/mL stock in | Sigma-Aldrich, Cat. No. |
| | H₂O | T7660 |
| | aliquot and store at −20° C. | |

Materials

| | |
|---|---|
| Corning ® BioCoat ™ Poly-D-Lysine | Cat. No. 354663, Lot |
| 384-well black, transparent, flat | No. 31616006 |
| bottom tissue culture plates | |
| Corning ® 384-well microplate, clear | Costar Cat. No.: 3656 |
| polypropylene, round bottom, sterile | |
| FLIPR pipette tips, 384-well | Molecular Devices, Cat. |
| | No. 9000-0764 |
| FLIPR Potassium Assay Kit | Molecular Devices, Cat. |
| | No. R8223 |

Instruments and Equipment
Nuaire cell culture hood, Cat. No. 540-600
37° C./5% CO incubator link to robotic arm, Liconic: STX110
Molecular Devices FLIPR^{Tetra} High throughput cellular screening system, Cat. No. FT0324, Molecular Devices
ThermoFisher MultiDrop 384, Cat. No. 5840300
Biotek Microfill, Cat. No. ASF1000A-4145
BioRad TC10 cell counter, Cat. No. 145-0010

### Assay Procedures

Cells Scaled Up from Frozen Vials
APOL1 G1 3.25 (HEK293 T-Rex) frozen vials: 5 million cells per vial
Step 1, Day 1: Defrost frozen vial into T-225.
Step 2, Day 5: (when 85% confluent): Split one T-225 at 3×10⁶ cells per flask.
Step 3, Day 8: Splits cells to set up for the assay plates as described below.

Cell Culture

T-Rex APOL1 HEK cells are split twice per week to keep the confluence state below 85% of the culture flask surface area. Cells can be kept until passage 25.
Cell Culture Medium
   DMEM high glucose+10% FBS, +P/S, +5 μg/mL blasticidin, +1 μg/mL puromycin.
   500 mL DMEM, +55 mL FBS, +5 mL P/S, +280 μL blasticidin 10 mg/mL, +56 μL Puromycin 10 mg/mL.
Assay Media
   Opti-MEM reduced serum medium from Invitrogen.

Day 1

Preparation of Cell Assay Plates
Culture medium is removed from the x cm² T-flask by aspiration.
The cell monolayer is rinsed with PBS 1× at room temperature. PBS is removed by aspiration.
Cells are trypsinized using Trypsin.
The flasks are incubated at room temperature for 2-3 minutes.
Complete DMEM medium is then added. Cell suspension is then transferred to a 50 mL Falcon polypropylene tube.
Cells are then counted using a BioRad TC10 cell counter and the required amount of cells are centrifuged at 1200 RPM for 5 minutes. Required amount is 1.3×10⁶ cells/mL APOL1 T-Rex HEK cells.
The pellet is suspended in the assay medium.
Using the MultiDrop, add 20 μL to each well (corresponds to 26000 cells total per well) of a 384-well black, transparent, flat bottom Poly-D coated plate.
Tetracycline as prepared in the following section is added to the cells before plating to induce APOL1 expression.
Plates are left at room temperature for 20 to 30 minutes before incubation at 37° C. and 5% CO₂.

Preparation of Tetracycline
Tetracycline stock is prepared at 1 mg/mL in H₂O, aliquoted and stored at −20° C.
On the day the cells are plated for the assay, the tetracycline working concentration is prepared as follows:
   Predilute tetracycline stock at 100× by transferring 50 μL stock in 5 mL assay media to give 10 μg/mL intermediate stock.
   Prepare tetracycline at 4× if added with Biomek to the cell plates or added directly on cells to give a 1× tetracycline concentration according to Table 15 below.

TABLE 15
Concentration of Tetracycline for cell plate.

| | 1X Tet | 5X Tet | mL | mL diluted cell |
|---|---|---|---|---|
| Clones | ng/mL | ng/mL | predilution | suspension |
| G1 DC3.25 | 15 | 75 | 0.3 | 39.7 |

Day 2

Preparation of Thallium Loading Dye and Cells Loading

FLIPR@ Potassium Assay Kit R8223
Preparation of the Loading Buffer:
   1. Remove one vial each of Component A (Dye) and Component C (Pluronic) from the freezer, and then equilibrate to room temperature.
   2. For the Bulk Kit, prepare 200 mL of 20 mM HEPES plus 1×HBSS, pH 7.4 as Component B.
   3. Dissolve the contents of the Component C vial in DMSO, and the mix thoroughly by vortexing.
   4. Combine the vial of Component A (dye) with 10 mL of the Component B buffer (HBSS 20 mM HEPES).
   5. Combine the Component C solution from step 3 to the Component A solution from step 4, and then mix by vortexing for 1 to 2 minutes until the contents of the vial are dissolved. Note: It is important that the contents are completely dissolved to ensure reproducibility between experiments.
   6. For the Bulk Kit only, combine the solution from step 5 with the remaining 190 mL of the prepared Component B buffer, and then mix thoroughly.
For each 10 mL of prepared dye add: 200 μL Probenecid (equals 2.5 mM final in assay plate) and 20 μL of 100 mM ouabain (equals 100 μM in assay plate).
Add 25 μL loading dye to each well of assay plate containing 25 μL. Link to robotic arm (with multidrop or microfill).
Incubate for 30 minutes at room temperature.

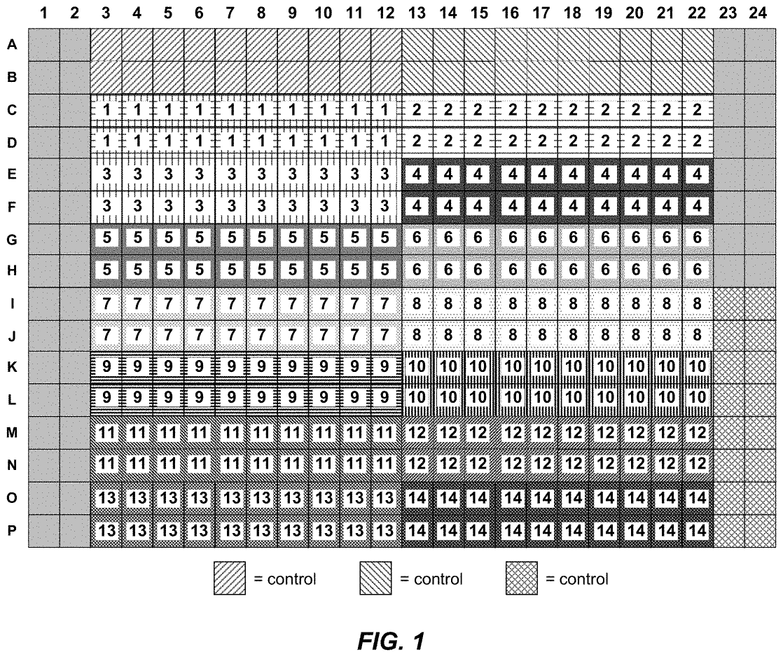

Preparation of Drug Plates and Transfer of Compounds to Assay Plates
The compounds are plated in assay ready plates (ARP). The plate layout in FIG. 1 shows the plate map for ARPs for dose response.
The compounds are hydrated with 20 μL HBSS with 20 mM HEPES.
The compounds are transferred to the assay plates 30 minutes after loading thallium sensitive dye as described in Preparation of Thallium Loading Dye described above.
The compounds are diluted by a 1:500 ratio for the final concentration.
The compound transfer is done using FLIPR. Mix: 3 strokes, 10 μL with speed @ 5 μL/sec, Height 20 μL. Aspirate: 10 μL with speed @ 5 μL/sec, Height 5 μL; Tip up speed of 20 mm/sec. Dispense: 10 μL with speed @ 5 μL/sec, Height 10 μL; liquid removal speed of 20 mm/sec.
Incubate for 30 minutes at room temperature.

Preparation of the Thallium Sulfate Source Plate
Prepare a 5× thallium sulfate solution in 1× chloride buffer.
For 5 mL of 5× thallium source plate: 1 mL of Chloride Free 5×, 0.5 mL Tl₂SO₄ 50 mM (2 mM equivalent final), 3.5 mL H₂O.
Dispense in 384-well Corning PP round-bottom plates (Costar, Cat. No. 3656).
Need 12.5 μL per well for each assay plate+dead volume.
Spin briefly.

Start Assay on FLIPR 384-Head

Parameters
Excitation: 470-495 nm; Emission: 515-575 nm.
Addition volume: 12.5 μL.
Aspirate: 12.5 μl with speed @ 20 μl/sec, Height 5 μl; Tip up speed of 20 mm/sec
Dispense: 12.5 μl with speed @ 20 μl/sec, Height 40 μl; liquid removal speed of 20 mm/sec.
Read baseline for 10 seconds; transfer 12.5 μL to assay plate.
Read every second for 60 seconds.
Keep tips on head for thallium addition.

Data Analysis
Stat file: Export slope (rate) between 17 and 32 seconds.
Analyze using (No Tet DMSO) and (Tet DMSO) controls (set up Stimulation and neutral controls, respectively).
Calculate percent inhibition thallium rate versus controls.
Data is reported as IC₅₀ (half maximum inhibitory concentration) and maximum percent inhibition.
   Trypanosoma brucei brucei Lysis Assay Using APOL1 Recombinant Protein

Trypanosoma brucei brucei is a blood stream parasite to which human, gorillas and baboon are immune due to the presence of the APOL1 protein in their HDL particles. The protein is uptaken by the parasite via the TbHpHb receptor located in its flagellar pocket and is bonded by the Hpr protein contained in the HDL particles which triggers the receptor endocytosis by the parasite.

Following endocytosis, the formed vesicle containing the HDL particle matures from early to late endosome, and subsequently to lysosome. The concomitant pH change in the lumen of the vesicle triggers the insertion of the APOL1 protein into the membrane of the late endosome/lysosome and hereby triggers lysosomal membrane permeabilization and as a further downstream event, trypanosome lysis. Trypanosoma brucei brucei is sensitive to lysis by all three APOL1 variants (G0, G1, and G2).

The Trypanosoma brucei brucei lysis assay is a lysis assay of the parasite using recombinant APOL1 protein variant followed by a fluorescent detection method of viability by the addition of AlamarBlue reagent to the assay well, a general metabolic redox indicator (AlamarBlue assay).

Briefly, the AlamarBlue active compound, the resazurin, a blue, water soluble, non-toxic and cell permeable molecule, which can be followed by absorbance, is reduced by various metabolic pathways into resorufin, a red compound which can be followed by either absorbance or fluorescence. The assay allows the calculation of the percent viability (percent of living Trypanosomes remaining in each well) at the end of a lysis relative to the untreated condition by interpolation of fluorescent values (FLU) on a standard curve with a known amount of seeded trypanosome/well.

### Reagents and Materials

Trypanosoma brucei brucei (ATCC, Cat. No. PRA-382)
Lister 427 VSG 221 bloodstream form.

Thaw/Expansion Media (ATCC Medium 2834 Modified HMI-9 Medium)

| | | |
|---|---|---|
| IMDM | 250 mL | 76.3% |
| FBS | 25 mL | 7.63% |
| Serum Plus | 25 mL | 7.63% |
| HMI-9 | 25 mL | 7.63% |
| Hypoxanthine | 2.5 mL | 0.763% |
| | 327.5 mL total | |

Assay Media (No Phenol Red/No FBS): Make on Day of Use

| | | |
|---|---|---|
| IMDM No Phenol Red | 250 mL | 82.6% |
| Serum Plus | 25 mL | 8.26% |
| HMI-9 | 25 mL | 8.26% |
| Hypoxanthine | 2.5 mL | 0.826% |
| | 302.5 mL total | |

HMI-9 (10×)

| | | |
|---|---|---|
| Bathocuproine disulfonic acid | 280 | mg |
| Cysteine | 1820 | mg |
| Sodium pyruvate (100x) | 100 | mL |
| Uracil | 100 | mg |
| Cytosine | 100 | mg |
| 2-mercaptoethanol | 140 | μL |
| Water | 900 | mL |
| | 1000 | mL total |

Hypoxanthine Stock (100×)-9 (10×)

| | | |
|---|---|---|
| Sodium Hydroxide | 0.8 | g |
| Hypoxanthine | 2.72 | g |
| Water | 200 | mL |
| | 200 | mL total |

Media Reagents

| | | |
|---|---|---|
| IMDM | Phenol Red | Life Technologies, Cat. |
| | sodium pyruvate | No. 12440 |
| | L-glutamine | |
| | 25 mM HEPES | |
| IMDM | NO Phenol Red | Life Technologies, Cat. |
| | sodium pyruvate | No. 21056 |
| | L-glutamine | |
| | 25 mM HEPES | |
| FBS | Heat inactivated | Sigma-Aldrich, Cat. No. |
| | | F8317-500 mL |
| Serum Plus | medium | Sigma-Aldrich, Cat. No. |
| | supplement | 14008C |
| Bathocuproine | | Sigma-Aldrich, Cat. No. |
| disulfonic acid | | B1125-1G |
| Cysteine | | Sigma-Aldrich, Cat. No. |
| | | C7352-25G |
| Sodium Pyruvate | 100x | Sigma-Aldrich, Cat. No. |
| Solution | | S8636-100ml |
| Uracil | | Sigma-Aldrich, Cat. No. |
| | | U1128-25G |
| Cytosine | | Sigma-Aldrich, Cat. No. |
| | | C3506-1G |
| 2-mercaptoethanol | | Sigma-Aldrich, Cat. No. |
| | | M3148-25ml |
| Hypoxanthine | | Sigma, Cat. No. H9636 |
| Sodium hydroxide | | Sigma-Aldrich, Cat. No. |
| | | S8045-500G |

Materials

| | | |
|---|---|---|
| T75/T175 | Nunc ™Non-Treated flask | T75 Thermo-Fisher |
| | Non-TC treated | Cat. No. 156800 |
| | Vented/White lids with | T175 Thermo-Fisher |
| | filter | Cat. No. 159926 |
| Assay Plates | 384 well black clear bottom | Corning ® Cat. |
| | Non-sterile | No. 3762 |
| | Non-TC treated | |
| Polypropylene | | Corning ® Cat. |
| storage plates | | No. 3656 |
| Plate Lids | Clear universal sterile lids | Thermo-Fisher Cat. |
| | | No. 250002 |
| Bravo Tips | 30 μL tips for 384 well | Axygen Cat. No. |
| | | VT-384-31UL-R-S |
| E1-Clip Tip pipette 12 | | Thermo-Fisher Cat. |
| channel adjustable | | No. 4672070BT |
| 2-125 μL | | |
| Tips | 125 μL E1-Clip sterile filter | Thermo-Fisher Cat. |
| | | No. 94420153 |
| Tips | 125 μL E1-Clip sterile | Thermo-Fisher Cat. |
| | (non-filter) | No. 94410153 |

Equipment
E1-Clip Tip pipette 12 channel adjustable 2-125 μL, Cat. No. 4672070BT
ThermoFisher MultiDrop 384, Cat. No. 5840300
Multidrop
Agilent Bravo, Cat. No. G5409A
Bravo
SpectraMax M5

Assay Ready Plates (ARPs)
ARPs comes in two formats:
   10 mM final top concentration with a 2.5 fold dilution down.
   5 mM final top concentration with a 3 fold dilution down.
      Both have a 10 point Dose response.
      0.1% DMSO final in the Black Assay Plate.
      Compounds are diluted 1000 fold in the Black Assay Plate.
      Each plate is designed for 14 compounds in duplicate.
In the final Black Assay Plate:
   Column 1: Media only (no APOL1) (100% viable)
   Column 2-23: 0.05 μg/mL APOL1 (˜EC₉₀) (10% viable with APOL1)
   Column 24: 0.1 μg/mL APOL1 (EC₁₀₀) (Approx. 0% viable)

### Assay Procedures

### Trypanosoma brucei brucei Culture

### Protocol A

Step 1, Day 1
That the cells at 35° C. for no more than 2 minutes.
Resuspend one vial gently in 20 mL pre-warmed media and incubate in a T75 flask at 37° C. and 5% CO₂.
Do not remove the cryoprotective agent.

Step 2, Day 4
Centrifuge at 800×g for 5 minutes at room temperature.
Resuspend in 1 mL media.
Make a 1:25 fold dilution (10 μL/240 μL media).
Count on a hemocytometer (after adding parasites).
   Let sit for 1-2 minutes for the parasites to settle.
   Count should be approximately 100 viable motile parasites/16 grid or approximately 25×10⁶ parasites/flask.
Passage the parasites by adding 1×10⁶ parasites/T75 flask in 20 mL media.
Passage the parasites by adding 2.33×10⁶ parasites/T175 flask in 46.6 mL media.
   For every T75 flask should make enough for approximately 1.5×384 well assay plates.
   For every T175 flask should make enough for approximately 3.8×384 well assay plates.

Step 3, Day 6
Centrifuge at 800×g for 5 minutes.
   Resuspend in 3 mL assay media (No phenol red, no FBS) per 75 starting flask.
   Resuspend in 7 mL assay media (No phenol red, no FBS) per 175 flask
Make a 1:25 fold dilution.
Count by hemocytometer.
   Every T75 flask set up should have approximately 75×10⁶ parasites/flask (verify doubling time=8.7 hours±1 hour).
   Every T175 flask set up should have approximately 175×10⁶ parasites/flask (verify doubling time=8.7 hours±1 hour).
   Require 46×10⁶ parasites per 384 well plate (at 120,000 parasites per well).

### Protocol B

Step 1, Day 1
Thaw the cells at 35° C. for not more than 2 minutes.
Resuspend one vial gently in 20 mL of pre-warmed mediate and incubate in a T75 flask at 37° C. and 5% CO₂.
Do not remove the cryoprotective agent.

Step 2, Day 2
Centrifuge at 800×g for 5 minutes at room temperature.
Resuspend in 1 mL media.
Make a 1:25 fold dilution (10 μL/240 μL media).
   Let sit for 1-2 minutes for the parasites to settle.
   Count should be approximately 100 viable motile parasites/16 grid or approximately 8×10⁶ parasites per flask.
Passage the parasites by adding 1.25×10⁶ parasites per T75 flask in 20 mL media.
   For every T75 flask set up should have approximately 1.5×384 well assay plates.
   For every T175 flask set up should have approximately 3.8×384 well assay plates.

Step 3, Day 5
Centrifuge at 800×g for 5 minutes.
   Resuspend in 3 mL assay media (No phenol red, no FBS) per T75 starting flask.
   Resuspend in 7 mL assay media (No phenol red, no FBS) per T175 starting flask.
Make a 1:25 fold dilution.
Count by hemocytometer.
   Every T75 flask should have approximately 75×10⁶ parasites per flask (verify doubling time: 7.7 hours±1 hour).
   Every T175 flask should have approximately 175×10⁶ parasites per flask (verify doubling time: 7.7 hours±1 hour).

### Lysis Assay Setup

APOL1 G1 Protein
Remove an aliquot of the 1.2 mg/mL APOL1 protein stock from −70° C.
Determine amount required for the experiment:
   Need 11.5 mL of 0.1 μg/mL APOL1 per 384 well plate.
   Need 0.5 mL of 0.2 μg/mL APOL1 per 384 well plate for control.
Make initial 1:10 dilution (10 μL/90 μL) into Assay media (now at 120 μg/mL).
   Using APOL1 at a final concentration of 0.05 μg/mL for an ˜EC₅₀. Need to determine this value for each new lot of protein used.
   Adding 30 mL/well of 2×APOL1 concentration of 0.1 μg/mL.
      Solution A: Measure 8.33 μL (120 μg/mL) in 10 mL for a 0.1 μg/mL 2× stock.
      Solution B: Measure 16.67 μL (120 μg/mL) in 10 mL for a 0.2 μg/mL 2× stock control.

Multidrop
Black Assay Plate (384 well black well clear bottom, Cat. No. 3762).
   Column 1: Dispense 30 μL/well of Assay media (no APOL1).
   Column 2-23: Dispense 30 μL/well of Solution A (0.1 μg/mL APOL1).
   Column 24: Dispense 30 μL/well of Solution B (0.2 μg/mL APOL1).
Storage Plate (Polypropylene storage plate, Corning® Cat. No. 3656).
   Column 1-24: Dispense 80 μL Assay media (no APOL1) per well (30 mL media/plate).

Bravo: Compound Transfer
Place the storage plate, the Assay Ready Plate (ARP), and Black Assay Plate on the deck.
   Transfer 20 μL from the storage plate to the ARP and mix.
   Transfer 6 μL from the ARP to the Black Assay Plate and mix.
   Black Assay Plates are now ready for Trypanosome addition.

Trypanosome Addition:
Once the Black Assay Plates have compounds added, begin harvesting the

Trypanosomes as described in Step 3 of the Trypanosoma brucei brucei Culture section.
Count the Trypanosomes and prepare at 5×10⁶/mL in Assay media (No Phenol red and no FBS).
   Requires 9.2 mL of 5×10⁶ trypanosomes/mL for each 384 well plate (46×10⁶/plate).
Add 24 μL of 5×10⁶ trypanosomes mix to each well of a 384 well plate using the E1-Clip multichannel 12 channel 2-125 μL adjustable pipette.
Once addition is complete, tap plate on the surface to ensure liquid is within each well.
Place plates on the plate shaker for approximately 10 seconds and shake to ensure even distribution and that no drops are left on any edges.
Place in incubator overnight (16 hours) at 37° C. and 5% CO₂.
Each well should include 60 μL:
   30 μL 2×APOL1 media, 6 μL of 10× compounds, and 24 μL of trypanosome solution.

AlamarBlue Addition
After 16 hours overnight in incubator, remove required amount of AlamarBlue (2.3 mL/plate) from the bottle stored in refrigerator, and warm up briefly in a 37° C. water bath.
Add 6 μL/well using the E1-Clip Multichannel 12 channel 2-125 μL adjustable pipette.
Protect from light and incubate the plate at 37° C. and 5% CO₂ for 2.5 hrs.
   Read on SpectraMax (Softmax Pro 6.4 software, excitation: 555 nm, emission: 585 nm)

### Potency Data for Compounds 1 to 527

The compounds of Formula I, deuterated derivatives thereof and pharmaceutically acceptable salts of any of the foregoing are useful as inhibitors of APOL1 activity. Table 16 below illustrates the IC₅₀ of the compounds 1 to 527 using procedures described above (assays described above in Example 2A and 2B). In Table 16 below, the following meanings apply. For IC₅₀: “+++” means<0.25 μM; “++” means 0.25 μM to 1.0 μM; “+” means greater than 1.0 μM. N.D.=Not determined.

TABLE 16
Potency data for Compounds 1 to 527

| | Thallium Assay | Trypanosomal |
|---|---|---|
| Compound No. | (IC₅₀) | Assay (IC₅₀) (μM) |
| 1 | +++ | +++ |
| 2 | + | + |
| 3 | ++ | N.D. |
| 4 | +++ | +++ |
| 5 | + | + |
| 6 | ++ | ++ |
| 7 | + | + |
| 8 | +++ | +++ |
| 9 | +++ | +++ |
| 10 | + | + |
| 11 | ++ | ++ |
| 12 | ++ | ++ |
| 13 | ++ | + |
| 14 | ++ | ++ |
| 15 | ++ | ++ |
| 16 | + | ++ |
| 17 | ++ | ++ |
| 18 | ++ | ++ |
| 19 | ++ | ++ |
| 20 | + | + |
| 21 | ++ | ++ |
| 22 | ++ | ++ |
| 23 | + | + |
| 24 | ++ | + |
| 25 | + | + |
| 26 | + | + |
| 27 | + | + |
| 28 | ++ | + |
| 29 | ++ | ++ |
| 30 | ++ | ++ |
| 31 | + | ++ |
| 32 | ++ | ++ |
| 33 | + | + |
| 34 | + | + |
| 35 | + | + |
| 36 | + | + |
| 37 | + | + |
| 38 | ++ | ++ |
| 39 | ++ | + |
| 40 | ++ | ++ |
| 41 | + | + |
| 42 | + | + |
| 43 | + | + |
| 44 | + | + |
| 45 | ++ | + |
| 46 | + | + |
| 47 | ++ | + |
| 48 | + | + |
| 49 | + | + |
| 50 | + | + |
| 51 | + | + |
| 52 | + | + |
| 53 | + | + |
| 54 | + | + |
| 55 | + | + |
| 56 | + | + |
| 57 | + | + |
| 58 | + | + |
| 59 | + | + |
| 60 | + | + |
| 61 | + | + |
| 62 | + | + |
| 63 | + | + |
| 64 | + | + |
| 65 | + | + |
| 66 | + | + |
| 67 | + | + |
| 68 | + | + |
| 69 | + | + |
| 70 | ++ | + |
| 71 | + | + |
| 72 | + | + |
| 73 | + | + |
| 74 | + | + |
| 75 | + | + |
| 76 | + | + |
| 77 | + | + |
| 78 | + | + |
| 79 | + | + |
| 80 | + | + |
| 81 | + | + |
| 82 | + | + |
| 83 | + | + |
| 84 | + | + |
| 85 | + | + |
| 86 | + | + |
| 87 | + | + |
| 88 | + | + |
| 89 | ++ | +++ |
| 90 | ++ | +++ |
| 91 | +++ | +++ |
| 92 | + | + |
| 93 | + | + |
| 94 | + | N.D. |
| 95 | ++ | ++ |
| 96 | ++ | N.D. |
| 97 | ++ | + |
| 98 | ++ | + |
| 99 | +++ | +++ |
| 100 | ++ | ++ |
| 101 | +++ | +++ |
| 102 | ++ | ++ |
| 103 | ++ | ++ |
| 104 | + | + |
| 105 | + | ++ |
| 106 | +++ | ++ |
| 107 | ++ | N.D. |
| 108 | + | + |
| 109 | ++ | ++ |
| 110 | ++ | N.D. |
| 111 | + | + |
| 112 | + | ++ |
| 113 | + | ++ |
| 114 | + | + |
| 115 | ++ | ++ |
| 116 | + | + |
| 117 | + | ++ |
| 118 | ++ | ++ |
| 119 | + | + |
| 120 | +++ | +++ |
| 121 | +++ | +++ |
| 122 | ++ | + |
| 123 | + | + |
| 124 | ++ | + |
| 125 | + | + |
| 126 | ++ | ++ |
| 127 | ++ | N.D. |
| 128 | N.D. | ++ |
| 129 | + | + |
| 130 | + | + |
| 131 | +++ | +++ |
| 132 | +++ | N.D. |
| 133 | ++ | + |
| 134 | + | + |
| 135 | + | + |
| 136 | + | + |
| 137 | + | + |
| 138 | + | + |
| 139 | + | + |
| 140 | + | + |
| 141 | ++ | ++ |
| 142 | + | + |
| 143 | + | + |
| 144 | + | + |
| 145 | + | + |
| 146 | + | + |
| 147 | + | N.D. |
| 148 | + | + |
| 149 | + | + |
| 150 | + | + |
| 151 | + | + |
| 152 | + | + |
| 153 | + | + |
| 154 | + | + |
| 155 | + | + |
| 156 | + | + |
| 157 | + | + |
| 158 | + | + |
| 159 | + | N.D. |
| 160 | + | + |
| 161 | + | + |
| 162 | + | + |
| 163 | + | + |
| 164 | + | + |
| 165 | + | + |
| 166 | + | + |
| 167 | + | + |
| 168 | + | + |
| 169 | + | + |
| 170 | + | + |
| 171 | + | + |
| 172 | + | + |
| 173 | + | + |
| 174 | + | + |
| 175 | + | + |
| 176 | + | + |
| 177 | + | + |
| 178 | + | + |
| 179 | + | + |
| 180 | + | + |
| 181 | + | + |
| 182 | + | + |
| 183 | + | + |
| 184 | + | + |
| 185 | + | + |
| 186 | + | + |
| 187 | +++ | +++ |
| 188 | +++ | ++ |
| 189 | + | + |
| 190 | + | + |
| 191 | + | + |
| 192 | + | + |
| 193 | + | + |
| 194 | ++ | N.D. |
| 195 | ++ | N.D. |
| 196 | ++ | N.D. |
| 197 | ++ | N.D. |
| 198 | ++ | N.D. |
| 199 | ++ | N.D. |
| 200 | ++ | N.D. |
| 201 | + | N.D. |
| 202 | + | N.D. |
| 203 | + | N.D. |
| 204 | + | N.D. |
| 205 | + | N.D. |
| 206 | + | N.D. |
| 207 | +++ | N.D. |
| 208 | +++ | N.D. |
| 209 | + | N.D. |
| 210 | + | N.D. |
| 211 | + | N.D. |
| 212 | ++ | N.D. |
| 213 | ++ | N.D. |
| 214 | +++ | N.D. |
| 215 | ++ | N.D. |
| 216 | ++ | N.D. |
| 217 | + | N.D. |
| 218 | + | N.D. |
| 219 | + | N.D. |
| 220 | + | N.D. |
| 221 | + | N.D. |
| 222 | + | N.D. |
| 223 | + | N.D. |
| 224 | + | N.D. |
| 225 | + | N.D. |
| 226 | + | N.D. |
| 227 | +++ | +++ |
| 228 | + | + |
| 229 | ++ | +++ |
| 230 | + | N.D. |
| 231 | +++ | N.D. |
| 232 | ++ | ++ |
| 233 | ++ | + |
| 234 | +++ | +++ |
| 235 | +++ | N.D. |
| 236 | + | + |
| 237 | N.D. | + |
| 238 | + | + |
| 239 | ++ | N.D. |
| 240 | ++ | N.D. |
| 241 | ++ | N.D. |
| 242 | + | N.D. |
| 243 | + | N.D. |
| 244 | + | N.D. |
| 245 | + | N.D. |
| 246 | + | N.D. |
| 247 | + | N.D. |
| 248 | + | N.D. |
| 249 | + | N.D. |
| 250 | + | N.D. |
| 251 | + | N.D. |
| 252 | + | N.D. |
| 253 | + | N.D. |
| 254 | + | N.D. |
| 255 | + | N.D. |
| 256 | + | N.D. |
| 257 | + | N.D. |
| 258 | + | N.D. |
| 259 | ++ | N.D. |
| 260 | + | N.D. |
| 261 | ++ | N.D. |
| 262 | +++ | N.D. |
| 263 | + | N.D. |
| 264 | + | N.D. |
| 265 | + | N.D. |
| 266 | + | N.D. |
| 267 | + | N.D. |
| 268 | + | N.D. |
| 269 | + | N.D. |
| 270 | + | N.D. |
| 271 | ++ | N.D. |
| 272 | + | N.D. |
| 273 | + | N.D. |
| 274 | ++ | N.D. |
| 275 | + | N.D. |
| 276 | + | N.D. |
| 277 | + | N.D. |
| 278 | + | N.D. |
| 279 | + | N.D. |
| 280 | + | N.D. |
| 281 | + | N.D. |
| 282 | ++ | N.D. |
| 283 | +++ | +++ |
| 284 | + | N.D. |
| 285 | + | N.D. |
| 286 | + | N.D. |
| 287 | + | N.D. |
| 288 | + | N.D. |
| 289 | + | N.D. |
| 290 | + | N.D. |
| 291 | + | N.D. |
| 292 | + | N.D. |
| 293 | + | N.D. |
| 294 | + | N.D. |
| 295 | + | N.D. |
| 296 | + | N.D. |
| 297 | + | N.D. |
| 298 | + | N.D. |
| 299 | + | N.D. |
| 300 | + | N.D. |
| 301 | +++ | N.D. |
| 302 | + | N.D. |
| 303 | + | N.D. |
| 304 | ++ | N.D. |
| 305 | + | N.D. |
| 306 | + | N.D. |
| 307 | + | N.D. |
| 308 | + | N.D. |
| 309 | + | N.D. |
| 310 | ++ | N.D. |
| 311 | + | N.D. |
| 312 | + | N.D. |
| 313 | + | N.D. |
| 314 | + | N.D. |
| 315 | + | N.D. |
| 316 | + | N.D. |
| 317 | + | N.D. |
| 318 | + | N.D. |
| 319 | + | N.D. |
| 320 | + | N.D. |
| 321 | + | N.D. |
| 322 | + | N.D. |
| 323 | + | N.D. |
| 324 | + | N.D. |
| 325 | + | N.D. |
| 326 | ++ | N.D. |
| 327 | ++ | N.D. |
| 328 | + | N.D. |
| 329 | + | N.D. |
| 330 | + | N.D. |
| 331 | + | N.D. |
| 332 | + | N.D. |
| 333 | +++ | N.D. |
| 334 | + | N.D. |
| 335 | + | N.D. |
| 336 | + | N.D. |
| 337 | + | N.D. |
| 338 | + | N.D. |
| 339 | ++ | N.D. |
| 340 | + | N.D. |
| 341 | ++ | N.D. |
| 342 | ++ | N.D. |
| 343 | + | N.D. |
| 344 | + | N.D. |
| 345 | + | N.D. |
| 346 | ++ | N.D. |
| 347 | +++ | N.D. |
| 348 | + | N.D. |
| 349 | ++ | N.D. |
| 350 | + | N.D. |
| 351 | + | N.D. |
| 352 | + | N.D. |
| 353 | ++ | N.D. |
| 354 | + | N.D. |
| 355 | + | N.D. |
| 356 | + | N.D. |
| 357 | +++ | N.D. |
| 358 | + | N.D. |
| 359 | + | N.D. |
| 360 | ++ | N.D. |
| 361 | ++ | ++ |
| 362 | ++ | N.D. |
| 363 | + | N.D. |
| 364 | ++ | N.D. |
| 365 | + | N.D. |
| 366 | ++ | N.D. |
| 367 | + | N.D. |
| 368 | + | N.D. |
| 369 | + | N.D. |
| 370 | + | N.D. |
| 371 | + | N.D. |
| 372 | + | N.D. |
| 373 | + | N.D. |
| 374 | + | N.D. |
| 375 | + | N.D. |
| 376 | + | N.D. |
| 377 | + | N.D. |
| 378 | + | N.D. |
| 379 | + | N.D. |
| 380 | + | N.D. |
| 381 | + | N.D. |
| 382 | + | N.D. |
| 383 | + | N.D. |
| 384 | + | N.D. |
| 385 | + | N.D. |
| 386 | + | N.D. |
| 387 | + | N.D. |
| 388 | ++ | N.D. |
| 389 | + | N.D. |
| 390 | + | N.D. |
| 391 | + | N.D. |
| 392 | ++ | N.D. |
| 393 | + | N.D. |
| 394 | + | N.D. |
| 395 | + | N.D. |
| 396 | + | N.D. |
| 397 | + | N.D. |
| 398 | + | N.D. |
| 399 | + | N.D. |
| 400 | + | N.D. |
| 401 | + | N.D. |
| 402 | + | N.D. |
| 403 | + | N.D. |
| 404 | ++ | N.D. |
| 405 | ++ | N.D. |
| 406 | ++ | N.D. |
| 407 | + | N.D. |
| 408 | ++ | N.D. |
| 409 | + | N.D. |
| 410 | ++ | N.D. |
| 411 | ++ | N.D. |
| 412 | ++ | N.D. |
| 413 | + | N.D. |
| 414 | + | N.D. |
| 415 | ++ | N.D. |
| 416 | + | N.D. |
| 417 | + | N.D. |
| 418 | + | N.D. |
| 419 | + | N.D. |
| 420 | + | N.D. |
| 421 | + | N.D. |
| 422 | + | N.D. |
| 423 | + | N.D. |
| 424 | + | N.D. |
| 425 | + | N.D. |
| 426 | + | N.D. |
| 427 | + | N.D. |
| 428 | + | N.D. |
| 429 | + | N.D. |
| 430 | + | N.D. |
| 431 | N.D. | N.D. |
| 432 | + | N.D. |
| 433 | +++ | +++ |
| 434 | +++ | N.D. |
| 435 | +++ | N.D. |
| 436 | +++ | N.D. |
| 437 | +++ | N.D. |
| 438 | +++ | N.D. |
| 439 | +++ | N.D. |
| 440 | +++ | N.D. |
| 441 | +++ | N.D. |
| 442 | +++ | N.D. |
| 443 | +++ | N.D. |
| 444 | +++ | N.D. |
| 445 | +++ | N.D. |
| 446 | +++ | N.D. |
| 447 | +++ | N.D. |
| 448 | +++ | N.D. |
| 449 | +++ | N.D. |
| 450 | +++ | N.D. |
| 451 | +++ | N.D. |
| 452 | +++ | N.D. |
| 453 | ++ | N.D. |
| 454 | ++ | N.D. |
| 455 | ++ | N.D. |
| 456 | ++ | N.D. |
| 457 | ++ | N.D. |
| 458 | ++ | N.D. |
| 459 | ++ | N.D. |
| 460 | ++ | N.D. |
| 461 | ++ | N.D. |
| 462 | ++ | N.D. |
| 463 | ++ | N.D. |
| 464 | ++ | N.D. |
| 465 | ++ | N.D. |
| 466 | ++ | N.D. |
| 467 | ++ | N.D. |
| 468 | ++ | N.D. |
| 469 | ++ | N.D. |
| 470 | ++ | N.D. |
| 471 | ++ | N.D. |
| 472 | ++ | N.D. |
| 473 | ++ | N.D. |
| 474 | ++ | N.D. |
| 475 | + | N.D. |
| 476 | + | N.D. |
| 477 | + | N.D. |
| 478 | + | N.D. |
| 479 | + | N.D. |
| 480 | + | N.D. |
| 481 | + | N.D. |
| 482 | + | N.D. |
| 483 | + | N.D. |
| 484 | + | N.D. |
| 485 | + | N.D. |
| 486 | + | N.D. |
| 487 | + | N.D. |
| 488 | + | N.D. |
| 489 | + | N.D. |
| 490 | + | N.D. |
| 491 | + | N.D. |
| 492 | + | N.D. |
| 493 | + | N.D. |
| 494 | + | N.D. |
| 495 | + | N.D. |
| 496 | + | N.D. |
| 497 | + | N.D. |
| 498 | + | N.D. |
| 499 | N.D. | N.D. |
| 500 | N.D. | N.D. |
| 501 | N.D. | N.D. |
| 502 | N.D. | N.D. |
| 503 | + | N.D. |
| 504 | ++ | +++ |
| 505 | +++ | ++ |
| 506 | ++ | ++ |
| 507 | +++ | ++ |
| 508 | ++ | ++ |
| 509 | +++ | +++ |
| 510 | ++ | ++ |
| 511 | +++ | +++ |
| 512 | ++ | ++ |
| 513 | +++ | N.D. |
| 514 | + | N.D. |
| 515 | ++ | ++ |
| 516 | + | N.D. |
| 517 | +++ | +++ |
| 518 | +++ | +++ |
| 519 | +++ | N.D. |
| 520 | + | N.D. |
| 521 | + | N.D. |
| 522 | + | N.D. |
| 523 | + | N.D. |
| 524 | + | N.D. |
| 525 | + | N.D. |
| 526 | + | + |
| 527 | N.D. | N.D. |

### Other Embodiments

This disclosure provides merely exemplary embodiments of the disclosed subject matter. One skilled in the art will readily recognize from the disclosure and embodiments, that various changes, modifications and variations can be made therein without departing from the spirit and scope of the disclosure as defined in the following claims.

What is claimed is:

1. A compound, deuterated derivative, or pharmaceutically acceptable salt selected from compounds of Formula I: 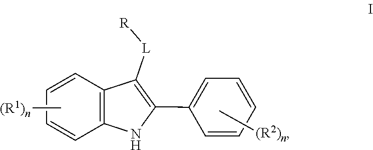
deuterated derivatives thereof, and pharmaceutically acceptable salts of any of the foregoing, wherein:
(i) R is selected from —C(O)NR³R⁴, —NR⁵C(O)R³, —NR⁵C(O)NR³R⁴, —NR³R⁴, —OR³, —NR⁵—SO₂R³, —OC(O)NR³R⁴, —C(O)OR³, 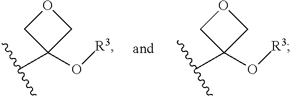
(ii) L is selected from divalent C₁-C₆ linear and branched alkyl, divalent C₂-C₆ linear and branched alkenyl, divalent C₂-C₆ linear and branched alkynyl, and divalent 1- to 7-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;
(iii) each R¹ is independently selected from:
halogen,
hydroxy,
thiol,
amino,
cyano,
C₁-C₆ linear, branched, and cyclic alkyl optionally substituted with 1-3 groups independently selected from hydroxy and halogen,
C₂-C₆ linear, branched, and cyclic alkenyl,
C₁-C₆ linear, branched, and cyclic alkoxy optionally substituted with 1-3 groups independently selected from halogen,
C₁-C₆ linear, branched, and cyclic thioalkyl optionally substituted with 1-3 groups independently selected from halogen, and
C₁-C₆ linear, branched, and cyclic aminoalkyl optionally substituted with 1-3 groups independently selected from halogen,
or two R¹ groups, together with the carbon atoms to which they are attached, may form a C₄-C₈ cycloalkyl, aryl, or heteroaryl;
(iv) each R² is independently selected from:
halogen,
hydroxy,
thiol,
amino,
cyano,
C₁-C₆ linear, branched, and cyclic alkyl optionally substituted with 1-3 groups independently selected from hydroxy and halogen,
C₂-C₄ linear, branched, and cyclic alkenyl,
C₁-C₆ linear, branched, and cyclic alkoxy optionally substituted with 1-3 groups independently selected from halogen,
C₁-C₄ linear, branched, and cyclic thioalkyl optionally substituted with 1-3 groups independently selected from halogen, and
C₁-C₄ linear, branched, and cyclic aminoalkyl optionally substituted with 1-3 groups independently selected from halogen,
(v) each n is independently selected from 0, 1, 2, 3, and 4;
(vi) R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl and C₃-C₆ cycloalkyl,
amide optionally substituted with 1-2 groups independently selected from C₁-C₃ alkyl,
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₃-C₆ cyclic alkyl optionally substituted with 1-2 groups independently selected from:
halogen,
hydroxy,
oxo,
amino optionally substituted with 1-2 groups independently selected from hydrogen and C₁-C₆ linear or branched alkyl,
aryl optionally substituted with 1-2 groups independently selected from halogen,
C₁-C₆ linear and branched alkyl groups (which may be further substituted with 1-3 groups independently selected from hydroxy, oxo, halogen, and C₁-C₆ linear and branched alkoxy groups),
carbamate optionally substituted with 1-2 groups independently selected from C₁-C₆ linear and branched alkyl,
C₁-C₆ linear and branched alkoxy, and
amide,
4- to 10-membered heterocyclyl optionally substituted with 1-3 groups independently selected from:
halogen,
oxo,
hydroxy,
amino, and
C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, and C₁-C₆ linear and branched alkyl (which may be further substituted with 1-2 groups independently selected from hydroxy and C₁-C₆ linear and branched alkoxy groups),
4- to 10-membered heteroaryl optionally substituted with 1-3 groups independently selected from:
amino,
hydroxy,
oxo,
halogen, and
C₁-C₆ linear alkyl (which may be further substituted with 1-3 groups independently selected from halogen and amino), and
C₁-C₆ linear and branched alkyl groups, wherein the alkyl groups are optionally substituted with 1-4 groups independently selected from:
amino groups optionally substituted with 1-2 groups independently selected from hydroxy, C₁-C₆ linear, branched, and cyclic alkyl (which may be further substituted with 1-2 oxo), and C₁-C₆ linear and branched alkylsulfonyl,
hydroxy,
oxo,
cyano,
carboxylic acid,
sulfonic acid,
—O-heteroaryl,
carbamate optionally substituted with 1-2 groups independently selected from C₁-C₆ linear and branched alkyl,
halogen,
amido optionally substituted with 1-2 groups independently selected from hydroxy, C₁-C₆ linear, branched, and cyclic alkyl groups, and C₁-C₆ linear, branched, and cyclic hydroxyalkyl,
C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
C₁-C₆ linear and branched alkynyl,
C₁-C₆ linear and branched alkoxy optionally substituted with 1-2 hydroxy,
C₁-C₆ linear and branched alkylsulfonyl,
aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
carbonyl-(4-methylpiperazin-1-yl),
carbonyl-(N-morpholino),
4- to 10-membered heterocyclyl optionally substituted with 1-3 groups independently selected from halogen, oxo, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-3 groups independently selected from halogen, oxo, hydroxy, and C₁-C₆ linear, branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl,
halogen,
hydroxy,
oxo,
C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
amide optionally substituted with 1-2 groups independently selected from C₁-C₆ linear and branched alkyl,
carbamate optionally substituted with 1-2 groups independently selected from C₁-C₆ linear and branched alkyl,
carboxamide optionally substituted with 1-2 groups independently selected from C₁-C₆ linear and branched alkyl,
C₁-C₆ linear and branched alkoxy groups optionally substituted with 1-2 groups independently selected from C₁-C₆ linear, branched, and cyclic alkyl and heterocyclyl,
4- to 10-membered heterocyclyl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups);
and
(vii) R⁵ is selected from hydrogen and C₁-C₆ linear or branched alkyl;
with the provisos that (1) the compound is not selected from 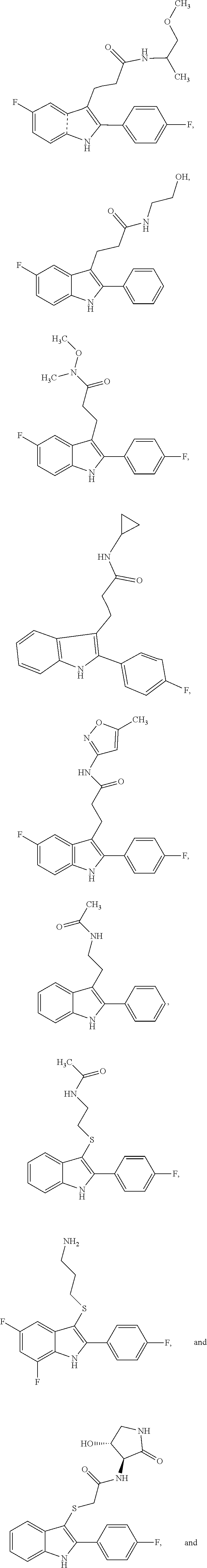
(2) when L is a divalent C₂ linear alkyl optionally substituted with 1-2 groups independently selected from methyl, halogen, and hydroxy and R is —NR³R⁴, then R³ and R⁴ are not

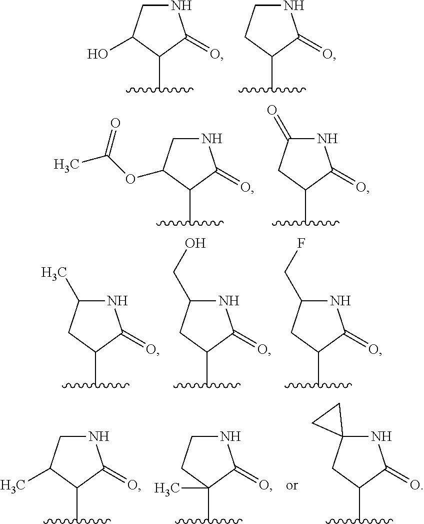

2. The compound, deuterated derivative, or pharmaceutically acceptable salt according to claim 1, wherein:
L is selected from divalent C₁-C₆ linear and branched alkyl, divalent C₂-C₆ linear and branched alkenyl, divalent C₂-C₆ linear and branched alkynyl, and divalent 1- to 6-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;
each R² is independently selected from:
halogen,
hydroxy,
thiol,
amino,
cyano,
C₁-C₄ linear, branched, and cyclic alkyl optionally substituted with 1-3 groups independently selected from hydroxy and halogen,
C₂-C₄ linear, branched, and cyclic alkenyl,
C₁-C₄ linear, branched, and cyclic alkoxy optionally substituted with 1-3 groups independently selected from halogen,
C₁-C₄ linear, branched, and cyclic thioalkyl optionally substituted with 1-3 groups independently selected from halogen, and
C₁-C₄ linear, branched, and cyclic aminoalkyl optionally substituted with 1-3 groups independently selected from halogen; and
R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl and C₃-C₆ cycloalkyl,
amide optionally substituted with 1-2 groups independently selected from C₁-C₃ alkyl, and
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₁-C₆ cyclic alkyl optionally substituted with 1-2 groups independently selected from:
halogen,
hydroxy,
oxo,
amino optionally substituted with 1-2 groups independently selected from hydrogen and C₁-C₆ linear or branched alkyl,
aryl optionally substituted with 1-2 groups independently selected from halogen,
C₁-C₆ linear and branched alkyl groups (which may be further substituted with 1-3 groups independently selected from hydroxy, oxo, halogen, and C₁-C₆ linear and branched alkoxy groups),
carbamate optionally substituted with 1-2 groups independently selected from C₁-C₆ linear and branched alkyl,
C₁-C₆ linear and branched alkoxy, and
amide,
4- to 10-membered heterocyclyl optionally substituted with 1-3 groups independently selected from:
halogen,
oxo,
hydroxy, and
C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, and C₁-C₆ linear and branched alkyl (which may be further substituted with one or two groups independently selected from hydroxy and C₁-C₆ linear and branched alkoxy groups),
4- to 10-membered heteroaryl optionally substituted with 1-3 groups independently selected from:
amino,
hydroxy,
oxo,
halogen, and
C₁-C₆ linear alkyl (which may be further substituted with 1-3 groups independently selected from halogen and amino),
C₁-C₆ linear and branched alkyl groups, wherein the alkyl groups are optionally substituted with 1-4 groups independently selected from:
amino groups optionally substituted with 1-2 groups independently selected from C₁-C₆ linear, branched, and cyclic alkyl (which may be further substituted with 1-2 oxo), and C₁-C₆ linear and branched alkylsulfonyl,
hydroxy,
oxo,
cyano,
carboxylic acid,
sulfonic acid,
—O-heteroaryl,
carbamate optionally substituted with 1-2 groups independently selected from C₁-C₆ linear and branched alkyl,
halogen,
amido optionally substituted with 1-2 groups independently selected from hydroxy, C₁-C₆ linear, branched, and cyclic alkyl groups and C₁-C₆ linear, branched, and cyclic hydroxyalkyl,
C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
C₁-C₆ linear and branched alkynyl,
C₁-C₆ linear and branched alkoxy optionally substituted with 1-2 hydroxy,
C₁-C₆ linear and branched alkylsulfonyl,
aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
carbonyl-(4-methylpiperazin-1-yl),
carbonyl-(N-morpholino),
4- to 10-membered heterocyclyl optionally substituted with 1-2 groups independently selected from halogen, oxo, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-3 groups independently selected from halogen, oxo, hydroxy, and C₁-C₆ linear, branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl,
halogen,
hydroxy,
oxo,
C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
amide optionally substituted with 1-2 groups independently selected from C₁-C₆ linear and branched alkyl,
carbamate optionally substituted with 1-2 groups independently selected from C₁-C₆ linear and branched alkyl,
carboxamide optionally substituted with 1-2 groups independently selected from C₁-C₆ linear and branched alkyl,
C₁-C₆ linear and branched alkoxy groups optionally substituted with 1-2 groups independently selected from C₁-C₆ linear, branched, and cyclic alkyl, and heterocyclyl,
4- to 10-membered heterocyclyl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups).

3. The compound, deuterated derivative, or pharmaceutically acceptable salt according to claim 1, wherein each R¹ is independently selected from halogen, hydroxy, amino, C₁-C₆ linear and branched alkyl (optionally substituted with 1-3 groups independently selected from hydroxy and halogen), C₃-C₆ cycloalkyl, and C₁-C₆ linear and branched alkoxy (optionally substituted with 1-3 groups independently selected from halogen).

4. The compound, deuterated derivative, or pharmaceutically acceptable salt according to claim 1, wherein each R² is independently selected from halogen, hydroxy, amino, cyano, C₁-C₆ linear and branched alkyl (optionally substituted with 1-3 groups independently selected from hydroxy and halogen), and C₁-C₆ linear and branched alkoxy (optionally substituted with 1-3 groups independently selected from halogen).

5. The compound, deuterated derivative, or pharmaceutically acceptable salt according to claim 1, wherein each R¹ and/or R² is fluorine.

6. The compound, deuterated derivative, or pharmaceutically acceptable salt according to claim 1, wherein each n is independently selected from 0, 1, and 2.

7. The compound, deuterated derivative, or pharmaceutically acceptable salt according to claim 1, wherein L is selected from divalent C₁-C₆ linear and branched alkyl, and divalent C₁-C₆ linear and branched thioalkyl, wherein the divalent alkyl and divalent thioalkyl are optionally substituted with 1-2 groups independently selected from halogen.

8. The compound, deuterated derivative, or pharmaceutically acceptable salt according to claim 7, wherein L is selected from divalent C₁-C₃ linear and branched alkyl, and divalent C₁-C₃ linear and branched thioalkyl, wherein the divalent alkyl and divalent thioalkyl are optionally substituted with 1-2 groups independently selected from halogen.

9. The compound, deuterated derivative, or pharmaceutically acceptable salt according to claim 1, wherein R is —C(O)NR³R⁴, and wherein R³ and R⁴ are independently selected from:
hydrogen,
C₁-C₆ linear and branched alkylsulfonyl,
C₁-C₆ linear and branched alkoxy optionally substituted with 1-2 groups independently selected from hydroxy and oxo;
C₃-C₆ cyclic alkyl optionally substituted with 1-2 groups independently selected from:
halogen,
hydroxy,
oxo,
amino,
aryl optionally substituted with 1-2 groups independently selected from halogen,
C₁-C₆ linear and branched alkyl groups (which may be further substituted with 1-3 groups independently selected from hydroxy and halogen),
carbamate optionally substituted with 1-2 groups independently selected from C₁-C₆ linear and branched alkyl,
C₁-C₆ linear and branched alkoxy, and
amido groups,
4- to 10-membered heterocyclyl optionally substituted with 1-3 groups independently selected from:
oxo,
hydroxy,
C₁-C₆ linear and branched alkyl (which may be further substituted with 1-2 groups independently selected from hydroxy, oxo, and C₁-C₆ linear and branched alkoxy),
4- to 10-membered heteroaryl optionally substituted with 1-3 groups independently selected from:
hydroxy,
oxo,
halogen, and
C₁-C₆ linear alkyl (which may be further substituted with 1-3 groups independently selected from halogen),
C₁-C₆ linear and branched alkyl groups, wherein the alkyl groups are optionally substituted with 1-4 groups independently selected from:
amino groups optionally substituted with 1-2 groups independently selected from C₁-C₆ linear, branched, and cyclic alkyl,
hydroxy,
oxo,
cyano,
carbamate optionally substituted with 1-2 groups independently selected from C₁-C₆ linear and branched alkyl,
halogen,
amido,
C₃-C₆ cyclic alkyl optionally substituted with 1-2 hydroxy,
4- to 10-membered heterocyclyl optionally substituted with 1-2 groups independently selected from oxo, hydroxy, and C₁-C₆ linear and branched alkyl (which may be further substituted with 1-2 hydroxy), and
4- to 10-membered heteroaryl optionally substituted with 1-3 groups independently selected from halogen and C₁-C₆ linear, branched, and cyclic alkyl (which may be further substituted with 1-3 groups independently selected from halogen),
or R³ and R⁴, together with the nitrogen atom to which they are attached, form a 4- to 10-membered heterocyclyl or heteroaryl optionally substituted with 1-3 groups independently selected from:
amino,
halogen,
hydroxy,
C₁-C₆ linear, branched, and cyclic alkyl optionally substituted with 1-2 groups independently selected from hydroxy, amino, and carbamate (which may be further substituted with 1-2 groups independently selected from C₁-C₆ linear and branched alkyl), and
carbamate optionally substituted with 1-2 groups independently selected from C₁-C₆ linear and branched alkyl.

10. The compound, deuterated derivative, or pharmaceutically acceptable salt according to claim 1, wherein R is —NR⁵—C(O)R³, and wherein R³ is selected from:
hydrogen,
C₁-C₆ linear and branched alkyl optionally substituted with 1-4 groups independently selected from halogen, hydroxy, cyano, amido (which may be further substituted by 1-2 groups independently selected from C₁-C₃ alkyl), amino (which may be further substituted with C₁-C₃ alkylsulfonyl), carbamate (which may be further substituted with C₁-C₆ linear and branched alkyl), 4- to 6-membered heterocyclyl (which may be further substituted with 1-2 groups independently selected from halogen, oxo, and hydroxy), 4- to 6-membered heteroaryl (which may be further substituted with 1-2 groups independently selected from halogen, oxo, hydroxy, and C₁-C₃ alkyl), and C₃-C₆ cycloalkyl (which may be further substituted with carbamate (which may be further substituted with C₁-C₆ linear or branched alkyl));
amide optionally substituted with 1-2 groups independently selected from C₁-C₃ alkyl,
C₁-C₆ linear and branched alkylsulfonyl,
C₁-C₆ linear and branched alkoxy optionally substituted with 1-2 groups independently selected from hydroxy, oxo, C₃-C₆ cycloalkyl (which may be further substituted with carboxylic acid), and 3- to 6-membered heteroaryl;
4- to 6-membered heterocyclyl optionally substituted with 1-2 groups independently selected from halogen, oxo, hydroxy, amino, and C₁-C₃ alkyl (which may be further substituted with 1-3 groups independently selected from halogen),
C₃-C₆ cycloalkyl optionally substituted with 1-2 groups independently selected from amide, hydroxy, halogen, C₁-C₆ linear and branched alkyl (which may be further substituted with 1-3 groups independently selected from halogen), and carbamate (which may be further substituted with C₁-C₆ linear and branched alkyl), and
4- to 6-membered heteroaryl optionally substituted with 1-2 groups independently selected from amino, halogen, oxo, hydroxy, and C₁-C₆ linear and branched alkyl (which may be further substituted with 1-3 groups independently selected from halogen);
and R⁵ is selected from hydrogen and C₁-C₃ linear or branched alkyl.

11. The compound, deuterated derivative, or pharmaceutically acceptable salt according to claim 10, wherein R⁵ is hydrogen.

12. The compound, deuterated derivative, or pharmaceutically acceptable salt according to claim 1, wherein R is —NR³R⁴, and wherein R³ and R⁴ are independently selected from:
4- to 6-membered heterocyclyl optionally substituted with 1-2 groups independently selected from hydroxy, halogen, and oxo,
C₁-C₃ alkyl optionally substituted with hydroxy, oxo, or halogen, and
hydrogen;
or R³ and R⁴, together with the nitrogen atom to which they are attached, form a 4- to 10-membered heterocyclyl optionally substituted with 1-3 groups independently selected from oxo and C₁-C₃ alkyl.

13. The compound, deuterated derivative, or pharmaceutically acceptable salt according to claim 1, wherein R is —OR³, and wherein R³ is selected from hydrogen and C₁-C₆ linear and branched alkyl.

14. The compound, deuterated derivative, or pharmaceutically acceptable salt according to claim 1, wherein R is —OC(O)NR³R⁴, and wherein R³ is selected from:
C₁-C₆ linear and branched alkyl optionally substituted with 1-4 groups independently selected from halogen, hydroxy, amide, cyano, C₃-C₆ cycloalkyl (which may be further substituted with hydroxy or C₁-C₃ alkoxy), 4- to 6-membered heteroaryl (which may be further substituted with C₁-C₃ alkyl, or trifluoro substituted C₁-C₃ alkyl), and 4- to 6-membered heterocyclyl (which may be further substituted with 1-3 groups independently selected from oxo and hydroxy),
C₁-C₆ linear and branched alkoxy,
C₃-C₆ cycloalkyl optionally substituted with 1-2 groups independently selected from hydroxy, halogen, amide, C₁-C₃ alkyl (which may be further substituted with hydroxy or halogen), and C₁-C₃ alkoxy,
4- to 6-membered heterocyclyl optionally substituted with 1-2 groups independently selected from hydroxy, halogen, oxo, and C₁-C₃ alkyl, and
4- to 6-membered heteroaryl optionally substituted with 1-2 groups independently selected from hydroxy, halogen, and oxo,
or R³ and R⁴, together with the nitrogen atom to which they are attached, form a 4- to 10-membered heterocyclyl optionally substituted with 1-3 groups independently selected from oxo, hydroxy, and C₁-C₃ alkyl.

15. The compound, deuterated derivative, or pharmaceutically acceptable salt according to claim 1, wherein R is —NR⁵—SO₂R³, and wherein R³ is selected from:
C₁-C₆ linear and branched alkyl optionally substituted with 1-2 groups independently selected from hydroxy, halogen, and 4- to 6-membered heterocyclyl (which may be further substituted with 1-2 groups independently selected from halogen, oxo, and hydroxy),
4- to 6-membered heterocyclyl,
4- to 6-membered heteroaryl optionally substituted with C₁-C₃ alkyl, and
amino optionally substituted with 1-2 groups independently selected from C₁-C₃ alkyl.

16. The compound, deuterated derivative, or pharmaceutically acceptable salt according to claim 1, wherein R is —C(O)OR³, and wherein R³ is selected from C₁-C₃ alkyl.

17. The compound, deuterated derivative, or pharmaceutically acceptable salt according to claim 1, wherein R is —NR⁵C(O)NR³R⁴, and wherein R³ and R⁴ are independently selected from:
C₁-C₆ linear and branched alkyl optionally substituted with 1-4 groups independently selected from halogen, hydroxy, oxo, cyano, amino (which may be further substituted with hydroxy), amido (which may be further substituted with hydroxy), sulfonic acid, aryl (optionally substituted with hydroxy), C₃-C₆ cycloalkyl (which may be further substituted 1-2 groups independently selected from hydroxy and C₁-C₃ hydroxyalkyl), and carboxylic acid,
C₃-C₆ cycloalkyl optionally substituted with 1-2 groups independently selected from halogen, hydroxy, and C₁-C₆ linear and branched alkyl (which may be further substituted with hydroxy),
4- to 6-membered heterocyclyl optionally substituted with 1-2 groups independently selected from halogen, oxo, and hydroxy, and
C₁-C₆ linear and branched alkylsulfonyl;
and R⁵ is selected from hydrogen and C₁-C₃ linear or branched alkyl.

18. The compound, deuterated derivative, or pharmaceutically acceptable salt according to claim 17, wherein R⁵ is hydrogen.

19. The compound, deuterated derivative, or pharmaceutically acceptable salt according to claim 1, wherein:
R is 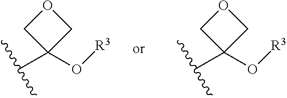
and
R³ is hydrogen.

20. A compound, deuterated derivative, or pharmaceutically acceptable salt selected from Compounds 1 to 527, deuterated derivatives thereof, or pharmaceutically acceptable salts of any of the foregoing.

21. A pharmaceutical composition comprising the compound, deuterated derivative, or pharmaceutically acceptable salt according to claim 1 and a pharmaceutically acceptable carrier.

22. A method of treating APOL1 mediated kidney disease comprising administering to a patient in need thereof the compound, deuterated derivative, or pharmaceutically acceptable salt according to claim 1.

23. The method according to claim 22, wherein the APOL1 mediated kidney disease is selected from ESKD, NDKD, FSGS, HIV-associated nephropathy, sickle cell nephropathy, diabetic neuropathy, arterionephrosclerosis, lupus nephritis, microalbuminuria, and chronic kidney disease.

24. The method according to claim 23, wherein the APOL1 mediated kidney disease is FSGS.

25. The method according to claim 23, wherein the APOL1 mediated kidney disease is NDKD.

26. The method according to claim 23, wherein the APOL1 mediated kidney disease is ESKD.

27. The method according to claim 22, wherein the APOL1 is associated with APOL1 genetic alleles chosen from homozygous G1: S342G:I384M and homozygous G2: N388del:Y389del.

28. The method according to claim 22, wherein the APOL1 is associated with compound heterozygous G1: S342G:I384M and G2: N388del:Y389del APOL1 alleles.

29. A method of treating APOL1 mediated kidney disease comprising administering to a patient in need thereof a compound, deuterated derivative, or pharmaceutically acceptable salt selected from compounds of Formula II: 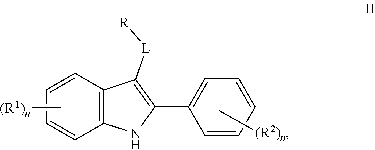
deuterated derivatives thereof, and pharmaceutically acceptable salts of any of the foregoing, wherein:
(i) R is selected from —C(O)NR³R⁴, —NR⁵C(O)R³, —NR⁵C(O)NR³R⁴, —NR³R⁴, —OR³, —NR⁵—SO₂R³, —OC(O)NR³R⁴, —C(O)OR³, 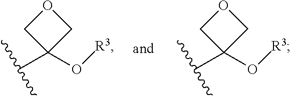
(ii) L is selected from divalent C₁-C₆ linear and branched alkyl, divalent C₂-C₆ linear and branched alkenyl, divalent C₂-C₆ linear and branched alkynyl, and divalent 1- to 7-membered heteroalkyl, wherein the divalent alkyl and divalent heteroalkyl are optionally substituted with 1-4 groups independently selected from:
C₁-C₆ alkyl,
aryl,
heteroaryl,
halogen,
hydroxy, and
amino;
(iii) each R¹ is independently selected from:
halogen,
hydroxy,
thiol,
amino,
cyano,
C₁-C₆ linear, branched, and cyclic alkyl optionally substituted with 1-3 groups independently selected from hydroxy and halogen,
C₂-C₆ linear, branched, and cyclic alkenyl,
C₁-C₆ linear, branched, and cyclic alkoxy optionally substituted with 1-3 groups independently selected from halogen,
C₁-C₆ linear, branched, and cyclic thioalkyl optionally substituted with 1-3 groups independently selected from halogen, and
C₁-C₆ linear, branched, and cyclic aminoalkyl optionally substituted with 1-3 groups independently selected from halogen,
or two R¹ groups, together with the carbon atoms to which they are attached, may form a C₄-C₈ cycloalkyl, aryl, or heteroaryl;
(iv) each R² is independently selected from:
halogen,
hydroxy,
thiol,
amino,
cyano,
C₁-C₆ linear, branched, and cyclic alkyl optionally substituted with 1-3 groups independently selected from hydroxy and halogen,
C₂-C₄ linear, branched, and cyclic alkenyl,
C₁-C₆ linear, branched, and cyclic alkoxy optionally substituted with 1-3 groups independently selected from halogen,
C₁-C₄ linear, branched, and cyclic thioalkyl optionally substituted with 1-3 groups independently selected from halogen, and
C₁-C₄ linear, branched, and cyclic aminoalkyl optionally substituted with 1-3 groups independently selected from halogen,
(v) each n is independently selected from 0, 1, 2, 3, and 4;
(vi) R³ and R⁴ are independently selected from:
hydrogen,
C₁-C₆ linear and branched alkylsulfonyl,
C₂-C₆ linear and branched alkenyl,
amino optionally substituted with 1-2 groups independently selected from C₁-C₆ linear and branched alkyl and C₃-C₆ cycloalkyl,
amide optionally substituted with 1-2 groups independently selected from C₁-C₃ alkyl,
C₁-C₆ linear and branched alkoxy optionally substituted with 1-2 groups independently selected from hydroxy, oxo, C₃-C₆ cyclic alkyl group (which may be further substituted with carboxylic acid), 3- to 6-membered heterocyclyl, and 3- to 6-membered heteroaryl;
C₃-C₆ cyclic alkyl optionally substituted with 1-2 groups independently selected from:
halogen,
hydroxy,
oxo,
amino optionally substituted with 1-2 groups independently selected from hydrogen and C₁-C₆ linear or branched alkyl,
aryl optionally substituted with 1-2 groups independently selected from halogen,
C₁-C₆ linear and branched alkyl groups (which may be further substituted with 1-3 groups independently selected from hydroxy, oxo, halogen, and C₁-C₆ linear and branched alkoxy groups),
carbamate optionally substituted with 1-2 groups independently selected from C₁-C₆ linear and branched alkyl,
C₁-C₆ linear and branched alkoxy, and
amide,
4- to 10-membered heterocyclyl optionally substituted with 1-3 groups independently selected from:
halogen,
oxo,
hydroxy,
amino, and
C₁-C₆ linear and branched alkyl (which may be further substituted with 1-2 groups independently selected from hydroxy, oxo, and C₁-C₆ linear and branched alkoxy),
aryl optionally substituted with 1-3 groups independently selected from halogen, hydroxy, and C₁-C₆ linear and branched alkyl (which may be further substituted with 1-2 groups independently selected from hydroxy and C₁-C₆ linear and branched alkoxy groups),
4- to 10-membered heteroaryl optionally substituted with 1-3 groups independently selected from:
amino,
hydroxy,
oxo,
halogen, and
C₁-C₆ linear alkyl (which may be further substituted with 1-3 groups independently selected from halogen and amino), and
C₁-C₆ linear and branched alkyl groups, wherein the alkyl groups are optionally substituted with 1-4 groups independently selected from:
amino groups optionally substituted with 1-2 groups independently selected from hydroxy, C₁-C₆ linear, branched, and cyclic alkyl (which may be further substituted with 1-2 oxo), and C₁-C₆ linear and branched alkylsulfonyl,
hydroxy,
oxo,
cyano,
carboxylic acid,
sulfonic acid,
—O-heteroaryl,
carbamate optionally substituted with 1-2 groups independently selected from C₁-C₆ linear and branched alkyl,
halogen,
amido optionally substituted with 1-2 groups independently selected from hydroxy, C₁-C₆ linear, branched, and cyclic alkyl groups, and C₁-C₆ linear, branched, and cyclic hydroxyalkyl,
C₃-C₆ cyclic alkyl optionally substituted with 1-2 groups independently selected from halogen, hydroxy, C₁-C₆ linear and branched hydroxyalkyl, C₁-C₆ linear and branched alkoxy, and carbamate (which may be further substituted with 1-2 groups independently selected from C₁-C₆ linear and branched alkyl),
C₁-C₆ linear and branched alkynyl,
C₁-C₆ linear and branched alkoxy optionally substituted with 1-2 hydroxy,
C₁-C₆ linear and branched alkylsulfonyl,
aryl optionally substituted with 1-2 groups independently selected from halogen groups, hydroxy, and C₁-C₆ linear and branched alkyl groups (which may be further substituted with 1-2 groups independently selected from hydroxy and C₁-C₆ linear and branched alkoxy groups),
carbonyl-(4-methylpiperazin-1-yl),
carbonyl-(N-morpholino),
4- to 10-membered heterocyclyl optionally substituted with 1-3 groups independently selected from halogen, oxo, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-3 groups independently selected from halogen, oxo, hydroxy, and C₁-C₆ linear, branched, and cyclic alkyl (which may be further substituted with 1-3 groups independently selected from halogen, hydroxy, and C₁-C₆ linear and branched alkoxy),
or R³ and R⁴, together with the nitrogen atom to which they are attached, form a 4- to 10-membered heterocyclyl or heteroaryl optionally substituted with 1-3 groups independently selected from:
amino optionally substituted with 1-2 groups independently selected from hydrogen and C₁-C₆ linear, branched, and cyclic alkyl,
halogen,
hydroxy,
oxo,
C₁-C₆ linear, branched, and cyclic alkyl optionally substituted with 1-2 groups independently selected from hydroxy, amino, C₁-C₆ linear, branched, and cyclic alkoxy groups, and carbamate (which may be further substituted with 1-2 groups independently selected from C₁-C₆ linear and branched alkyl),
amide optionally substituted with 1-2 groups independently selected from C₁-C₆ linear and branched alkyl,
carbamate optionally substituted with 1-2 groups independently selected from C₁-C₆ linear and branched alkyl,
carboxamide optionally substituted with 1-2 groups independently selected from C₁-C₆ linear and branched alkyl,
C₁-C₆ linear and branched alkoxy groups optionally substituted with 1-2 groups independently selected from C₁-C₆ linear, branched, and cyclic alkyl and heterocyclyl,
4- to 10-membered heterocyclyl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy), and
4- to 10-membered heteroaryl optionally substituted with 1-2 groups independently selected from halogen, hydroxy, and C₁-C₆ linear and branched alkyl (which may be further substituted with 1-2 groups independently selected from hydroxy and C₁-C₆ linear and branched alkoxy groups);
and
(vii) R⁵ is selected from hydrogen and C₁-C₆ linear or branched alkyl;
with the provisos that
(1) the compound is not 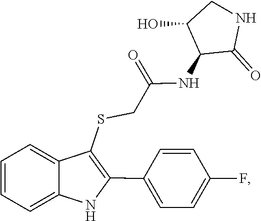 [SMILES: O=C(CSc1c(-c2ccc(F)cc2)[nH]c2ccccc12)N[C@@H]1C(=O)NC[C@H]1O]
and
(2) when L is a divalent C₂ linear alkyl optionally substituted with 1-2 groups independently selected from methyl, halogen, and hydroxy and R is —NR³R⁴, then R³ and R⁴ are not

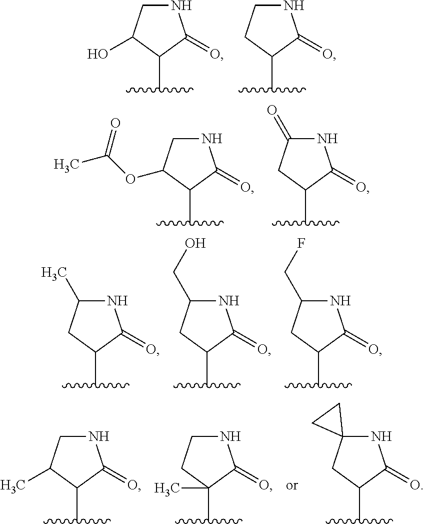

30. The method according to claim 29, wherein the APOL1 mediated kidney disease is chosen from ESKD, NDKD, FSGS, HIV-associated nephropathy, arterionephrosclerosis, lupus nephritis, microalbuminuria, and chronic kidney disease.

31. The method according to claim 29, wherein the APOL1 mediated kidney disease is FSGS.

32. The method according to claim 29, wherein the APOL1 mediated kidney disease is NDKD.

33. The method according to claim 29, wherein the APOL1 mediated kidney disease is ESKD.

34. The method according to claim 29, wherein the APOL1 mediated kidney disease is associated with APOL1 genetic alleles chosen from homozygous G1: S342G:I384M and homozygous G2: N388del:Y389del.

35. The method according to claim 29, wherein the APOL1 is associated with compound heterozygous G1: S342G:I384M and G2: N388del:Y389del APOL1 alleles.

36. A method of inhibiting APOL1 activity comprising contacting said APOL1 with a compound selected from Formula II, a deuterated derivative thereof, or a pharmaceutically acceptable salt of any of the foregoing.

37. The method according to claim 36, wherein the APOL1 is associated with APOL1 genetic alleles chosen from homozygous G1: S342G:I384M and homozygous G2: N388del:Y389del.

38. The method according to claim 36, wherein the APOL1 is associated with homozygous G1: S342G:I384M APOL1 alleles.

39. The method according to claim 36, wherein the APOL1 is associated with compound heterozygous G1: S342G:I384M and G2: N388del:Y389del APOL1 alleles.
